(19) United States
(12) Patent Application Publication
(10) Pub. No.: US 2025/0320284 A1
(43) Pub. Date: Oct. 16, 2025

(54) Novel Molecules for Therapy and Diagnosis

(71) Applicant: AC IMMUNE SA, Lausanne (CH)

(72) Inventors: Davide Basco, Lausanne (CH); Romain Christian Ollier, Lausanne (CH); Tamara Seredenin, Lausanne (CH)

(21) Appl. No.: 18/708,989

(22) PCT Filed: Nov. 16, 2022
(86) PCT No.: PCT/EP2022/082125

§ 371 Date: May 9, 2024

(30) Foreign Application Priority Data
Nov. 16, 2021 (EP) 21208651.6
Sep. 9, 2022 (EP) 22194983.7

(51) Int. Cl. C07K 16/18; A61K 39/00; A61P 25/00; A61P 29/00

(52) U.S. Cl. CPC C07K 16/18; A61P 25/00; A61P 29/00; A61K 2039/505

(57) ABSTRACT

The present invention is in the field of the adaptor protein apoptosis associated speck-like protein containing a caspase-recruitment domain (ASC), ASC-dependent inflammasomes and propagation of inflammation in disease. The invention relates to ASC binding molecules, in particular to anti-ASC antibodies or antigen-binding fragments thereof and uses thereof. The present invention provides diagnostic tools and means and methods to prevent, alleviate and/or treat a disease, a disorder and/or abnormality associated with ASC-dependent inflammasome activation in disease, in particular extracellular ASC and/or ASC specks which may be associated with multiple pathologies.

### FIELD OF THE INVENTION

Inflammasomes are multiprotein high molecular weight complexes that activate inflammatory caspases and the cytokine IL-1β release in response to pathogens and danger signals. They play a key role in inflammatory and immune response. These complexes assemble in response to various danger signals such as molecules from infectious agents (pathogen-associated molecular patterns, PAMPs) as well as altered host molecules, products of sterile inflammation and tissue damage and environmental factors (danger associated molecular patterns, DAMPs). The inflammasome family consists of NALP1-14, IPAF, and NAIP 1-6, with each family member providing specificity towards different PAMPs/DAMPs including nucleic acids, bacterial proteins, metabolites, protein aggregates, and the activity of toxins (Sharma and Kanneganti 2016). Inflammasomes are typically composed of a sensor (a cytosolic pattern-recognition receptor, PRR) and an adaptor protein called apoptosis associated speck-like protein containing a caspase-recruitment domain (CARD) (ASC, also known as PYCARD), and an effector such as the protease caspase-1 (Broz and Dixit 2016). ASC is a 22-kDa adapter protein with an N-terminal pyrin domain (PYD) and a C-terminal CARD. The multiprotein inflammasome oligomeric complexes are formed through homodimeric interactions between NLR's N-terminal pyrin and the ASC's N-terminal pyrin and the ASC's C-terminal CARD with the N-terminal CARD of pro-caspase-1. This facilitates ASC polymerization to form long helical filaments that are condensed into an intracellular macromolecular aggregate, known as ASC speck (Fernandes-Alnemri, Wu et al. 2007). This complex induces the activation of pro-caspase-1 into active caspase that cleaves the inactive pro-IL-1β and pro-IL-18 forms into bioactive cytokines that activate downstream inflammatory pathways. ASC (PYCARD) functions as a central adapter protein for multiple inflammasomes from the NLR (NLRP1, NLRP3, NLRP6, NLRP7, NLRC4, NLRC5, NAIP2, NAIP5 and NAIP6) family, the hematopoietic interferon (HIN) and absent in melanoma 2 (AIM2) (Guo, Callaway et al. 2015). The formation of ASC specks is best described for the NLRP3 inflammasome but evidence exists of ASC specks formation for other inflammasomes including NLRC4 (Franklin, Bossaller et al. 2014) and NLRP1 (Gong, Robinson et al. 2021).

Inside the cell the main function of ASC speck is the activation and regulation of caspase-1 activity. In addition to the intracellular function, NLRP3/ASC complexes exert multiple activities in the extracellular space where they are released upon pyroptotic cell death and remain active and stable (reviewed in Franklin, Latz et al. 2018). ASC specks can sustain inflammatory reaction in the extracellular space by recruiting pro-caspase-1 and IL-1β, they may provide an alternative mechanism for antigen-presentation, entrap microbes and cellular debris for subsequent clearance by neutrophils. Furthermore, ASC specks possess prion-like properties and can propagate inflammation to recipient phagocytic cells. ASC specks taken up by recipient cells can further aggregate cytosolic soluble ASC and are able to induce IL-1β production (Baroja-Mazo, Martin-Sanchez et al. 2014, Franklin, Bossaller et al. 2014).

Inflammasome activation is associated with pathogenesis of multiple inflammatory conditions, including autoimmune, autoinflammatory, metabolic and neurodegenerative diseases; and the presence of ASC specks was described in patient-derived material (reviewed in de Souza et al., 2021). In particular, extracellular ASC or ASC specks were detected in lungs from patients with inflammatory pulmonary diseases (Franklin, Bossaller et al. 2014), plasma (Baroja-Mazo, Martin-Sanchez et al. 2014), and serum (Rowczenio, Pathak et al. 2018) of patients with cryopyrin-associated periodic syndrome (CAPS), serum from cystic fibrosis and systemic autoinflammatory disease (SAID) (Scambler, Jarosz-Griffiths et al. 2019), serum of Schnitzler syndrome (Rowczenio, Pathak et al. 2018), myelodysplastic syndrome (Basiorka, McGraw et al. 2018), serum from psoriasis patients (Forouzandeh, Besen et al. 2020), serum of patients with non-alcoholic steatohepatitis (NASH) (Cyr, Keane et al. 2020), in atherosclerotic plaques (Paramel Varghese, Folkersen et al. 2016). In the CNS diseases, ASC or ASC specks were found in Alzheimer's disease brain tissue in the core of amyloid plaques (Venegas, Kumar et al. 2017), in the CSF of patients with traumatic brain injury (Adamczak, Dale et al. 2012), serum of patients with stroke (Kerr, Garcia-Contreras et al. 2018) and multiple sclerosis (Keane, Dietrich et al. 2018). Additional evidence suggest that ASC specks can be involved in pathogenesis of allergic asthma (Lee, Ishitsuka et al. 2021), systemic lupus erythematosus (SLE)(Franklin, Bossaller et al. 2014), some cancers (Protti and De Monte 2020), viral infections including HIV-1 (Ahmad, Mishra et al. 2018), SARS-CoV-2 (Rodrigues, de Sa et al. 2021, Toldo, Bussani et al. 2021) and hepatitis B virus (Xie, Ding et al. 2020).

The presence of extracellular ASC and ASC specks in multiple pathologies make them an interesting target for therapeutic intervention and for diagnostics. The study by Franklin (Franklin, Bossaller et al. 2014) proved that ASC specks are accessible to peripherally delivered antibodies in vivo after inflammasome activation. Furthermore, the use of anti-ASC mAb showed protection in the models of traumatic brain and spinal cord injury (de Rivero Vaccari, Lotocki et al. 2008, de Rivero Vaccari, Lotocki et al. 2009) and multiple sclerosis (Desu, Plastini et al. 2020). However, another study suggested that anti-ASC antibodies exacerbated the inflammatory response in crystal-induced peritonitis models (Franklin, Bossaller et al. 2014). The latter used polyclonal ASC antibodies which makes data interpretation and potential therapeutic development complicated. Therefore, additional work is needed to understand the efficacy of antibodies targeting different epitopes of ASC for prevention of propagation of inflammation.

Moreover, there is a need to identify and develop specific anti-ASC monoclonal antibodies with therapeutic potential for anti-inflammatory treatments and diagnosis. In addition, detection of inflammasome components as potential biomarker can be useful for patient identification, stratification, and longitudinal follow-up. Currently such diagnostic tools are scarce (the only assay reported by Keane, Dietrich et al. 2018). Therefore, discovery and development of specific high affinity monoclonal antibodies (mAbs) against different epitopes of ASC protein would enable exploration of biomarker potential of ASC. Furthermore, this would eventually lead to a better design of clinical trials and enable development of new therapeutics.

The present invention provides specific high affinity mAbs or their fragments and derivatives thereof that specifically bind to ASC or ASC specks for use as anti-inflammatory treatments and diagnostics for diseases associated with inflammasome activation and propagation. The mAbs of the present invention target different epitopes of ASC and are capable of inhibiting ASC polymerization and propagation of inflammation in vitro and in vivo. Such mAbs are beneficial in the treatment of disease, disorder, or abnormality associated with accumulation of extracellular ASC specks. Antibodies against ASC are expected to neutralize extracellular ASC specks and subsequently dampen propagation of inflammatory signaling and ultimately provide functional improvement.

### PRIOR ART

WO2019122270 relates to neurodegenerative diseases and ligands interacting with the apoptosis-associated speck-like protein containing a CARD.

US2009104200 relates to modulating inflammasome activity and inflammation in the central nervous system and describes antibodies that specifically bind to at least one component (e.g., ASC, NALP1) in a mammalian inflammasome (e.g., the NALP1 inflammasome).

### SUMMARY OF THE INVENTION

In one aspect, there is provided an ASC binding molecule that binds an ASC speck and/or non-polymerized ASC.

In one embodiment, the ASC binding molecule binds preferentially ASC specks over non-polymerized ASC. In one embodiment, the ASC binding molecule binds preferentially non-polymerized ASC over ASC specks. In one embodiment, the ASC binding molecule binds ASC specks and does not bind to non-polymerized ASC. In one embodiment, the ASC binding molecule binds non-polymerized ASC and does not bind to ASC specks.

In one embodiment, the ASC binding molecule prevents or inhibits ASC polymerization. In one embodiment, the ASC polymerization is measured in vitro, preferably by an ASC polymerization assay. In one embodiment, the ASC binding molecule prevents or inhibits propagation of ASC-dependent inflammation. In one embodiment, the propagation of inflammation is measured in vitro or in vivo.

In one embodiment, the prevention or inhibition of propagation of inflammation is prevention or inhibition of IL-1β release. In one embodiment, the IL-1β release is measured in vitro, preferably in an assay employing phagocytic cells such as macrophages or microglia.

In one embodiment, the ASC binding molecule increases the uptake of ASC extracellular specks by phagocytic cells such as macrophages or microglia.

In one embodiment, the ASC binding molecule prevents or inhibits accumulation of ASC and/or ASC specks.

In one embodiment, the ASC or ASC speck accumulation is intracellular or extracellular.

In one embodiment, the ASC binding molecule binds to an epitope of human ASC of SEQ ID NO: 1; and/or mouse ASC of SEQ ID NO: 2. In one embodiment, the epitope is in the ASC PYD domain or ASC CARD domain.

In one embodiment, the ASC binding molecule prevents, reduces or inhibits demyelination.

In one embodiment, prevention, reduction, or inhibition of demyelination is improving demyelination score in vivo.

In one embodiment, the ASC binding molecule increases the spleen mass in vivo.

In one embodiment, the ASC binding molecule reduces levels of reactive microglia in vivo.

In one embodiment, the ASC binding molecule reduces levels of ASC and/or cleaved capase-1 protein in vivo.

In one embodiment, the ASC binding molecule binds to an epitope in the ASC PYD domain comprising, consisting essentially of, or consisting of amino acid residues:
a) L9, D10, E13, N14, E18 and E19,
b) L9, D10, E13 and N14,
c) E13 andN14,
d) Q79, E80, G83 and Q84; or
e) E18, E19, V30, P31, N71, R74, D75, G77, Q79 and E80.

The amino acids are stated with reference to the amino acid sequence of human ASC of SEQ ID NO: 1.

The ASC binding molecule may bind to an epitope in the ASC PYD domain comprising, consisting essentially of, or consisting of amino acid residues numbered:
a) 9, 10, 13, 14, 18 and 19,
b) 9, 10, 13 and 14,
c) 13 and 14,
d) 79, 80, 83 and 84, or
e) 18, 19, 30, 31, 71, 74, 75, 77, 79 and 80
   of human ASC of SEQ ID NO: 1.

As described in example 11, the epitopes may be defined using alanine scanning mutagenesis. Mutants of ASC, in particular the PYD domain of PYCARD, may be employed. Binding of the ASC binding molecules to mutants may be measured by a suitable immunoassay, such as an ELISA. The residues listed are those critical to binding, which may be defined as any appropriate loss of binding, such as retaining no more than 30% binding compared to a wild type control, in the presence of an alanine mutation at that position.

Alternatively, the ASC binding molecules may bind to an epitope in the ASC CARD domain comprising, consisting essentially of, or consisting of amino acid residues:
a. K174 and D175,
b. 1115, D116, R119, A120, K174, D175, S184, Q185, S186 and Y187,
c. 1115, D116, N170, W171, T172, K174, D175, S186 and Y187,
d. Y137,
e. R119, A120, L178, Q179, S186 and Y187 or
f. R119, A120, K174, D175, S186 and Y187.

The amino acids are stated with reference to the amino acid sequence of human ASC of SEQ ID NO: 1. The ASC binding molecule may bind to an epitope in the ASC CARD domain comprising, consisting essentially of, or consisting of amino acid residues numbered:
a. 174, 175,
b. 115, 116, 119, 120, 174, 175, 184, 186 and 187,
c. 115, 116, 170, 171, 172, 174, 175, 186 and 187,
d. 137,
e. 119, 120, 178, 179, 186 and 187 or
f. 119, 120, 174, 175, 186 and 187.
   of human ASC of SEQ ID NO: 1.

As described in example 13, the epitopes may be defined using alanine mutagenesis. Mutants of ASC, in particular the CARD domain of PYCARD, may be employed. Binding of the ASC binding molecules to mutants may be measured by a suitable immunoassay, such as an ELISA. The residues listed are those critical to binding, which may be defined as any appropriate loss of binding, such as retaining no more than 30% binding compared to a wild type control, in the presence of an alanine mutation at that position.

In one embodiment the ASC binding molecule of the invention comprises:
a. a VH-CDR1 comprising the amino acid sequence of SEQ ID NO: 11, a VH-CDR2 comprising the amino acid sequence of SEQ ID NO: 12, a VH-CDR3 comprising the amino acid sequence NEV (Asn-Glu-Val), a VL-CDR1 comprising the amino sequence SEQ ID NO: 15, a VL-CDR2 comprising the amino acid sequence of SEQ ID NO: 16, and a VL-CDR3 comprising the amino acid sequence of SEQ ID NO: 17; or
b. a VH-CDR1 comprising the amino acid sequence of SEQ ID NO: 21, a VH-CDR2 comprising the amino acid sequence of SEQ ID NO: 22, a VH-CDR3 comprising the amino acid sequence SEQ ID NO: 23, a VL-CDR1 comprising the amino sequence SEQ ID NO: 25, a VL-CDR2 comprising the amino acid sequence of SEQ ID NO: 26, and a VL-CDR3 comprising the amino acid sequence of SEQ ID NO: 27; or
c. a VH-CDR1 comprising the amino acid sequence of SEQ ID NO: 31, a VH-CDR2 comprising the amino acid sequence of SEQ ID NO: 32, a VH-CDR3 comprising the amino acid sequence of SEQ ID NO: 33, a VL-CDR1 comprising the amino sequence SEQ ID NO: 35, a VL-CDR2 comprising the amino acid sequence of SEQ ID NO: 36, and a VL-CDR3 comprising the amino acid sequence of SEQ ID NO: 37; or
d. a VH-CDR1 comprising the amino acid sequence of SEQ ID NO: 41, a VH-CDR2 comprising the amino acid sequence of SEQ ID NO: 42, a VH-CDR3 comprising the amino acid sequence SEQ ID NO: 43, a VL-CDR1 comprising the amino sequence SEQ ID NO: 45, a VL-CDR2 comprising the amino acid sequence of SEQ ID NO: 46, and a VL-CDR3 comprising the amino acid sequence of SEQ ID NO: 47; or
e. a VH-CDR1 comprising the amino acid sequence of SEQ ID NO: 51, a VH-CDR2 comprising the amino acid sequence of SEQ ID NO: 52, a VH-CDR3 comprising the amino acid sequence SEQ ID NO: 53, a VL-CDR1 comprising the amino sequence SEQ ID NO: 55, a VL-CDR2 comprising the amino acid sequence of SEQ ID NO: 56, and a VL-CDR3 comprising the amino acid sequence of SEQ ID NO: 57; or
f. a VH-CDR1 comprising the amino acid sequence of SEQ ID NO: 61, a VH-CDR2 comprising the amino acid sequence of SEQ ID NO: 62, a VH-CDR3 comprising the amino acid sequence SEQ ID NO: 63, a VL-CDR1 comprising the amino sequence SEQ ID NO: 65, a VL-CDR2 comprising the amino acid sequence of SEQ ID NO: 66, and a VL-CDR3 comprising the amino acid sequence of SEQ ID NO: 67; or
g. a VH-CDR1 comprising the amino acid sequence of SEQ ID NO: 71, a VH-CDR2 comprising the amino acid sequence of SEQ ID NO: 72, a VH-CDR3 comprising the amino acid sequence SEQ ID NO: 73, a VL-CDR 1 comprising the amino sequence SEQ ID NO: 75, a VL-CDR2 comprising the amino acid sequence of SEQ ID NO: 76, and a VL-CDR3 comprising the amino acid sequence of SEQ ID NO: 77; or
h. a VH-CDR1 comprising the amino acid sequence of SEQ ID NO: 81, a VH-CDR2 comprising the amino acid sequence of SEQ ID NO: 82, a VH-CDR3 comprising the amino acid sequence SEQ ID NO: 83, a VL-CDR1 comprising the amino sequence SEQ ID NO: 85, a VL-CDR2 comprising the amino acid sequence of SEQ ID NO: 86, and a VL-CDR3 comprising the amino acid sequence of SEQ ID NO: 87; or
i. a VH-CDR1 comprising the amino acid sequence of SEQ ID NO: 91, a VH-CDR2 comprising the amino acid sequence of SEQ ID NO: 92, a VH-CDR3 comprising the amino acid sequence SEQ ID NO: 93, a VL-CDR1 comprising the amino sequence SEQ ID NO: 95, a VL-CDR2 comprising the amino acid sequence of SEQ ID NO: 96, and a VL-CDR3 comprising the amino acid sequence of SEQ ID NO: 97; or
j. a VH-CDR1 comprising the amino acid sequence of SEQ ID NO: 111, a VH-CDR2 comprising the amino acid sequence of SEQ ID NO: 112, a VH-CDR3 comprising the amino acid sequence SEQ ID NO: 113, a VL-CDR1 comprising the amino sequence SEQ ID NO: 115, a VL-CDR2 comprising the amino acid sequence of SEQ ID NO: 116, and a VL-CDR3 comprising the amino acid sequence of SEQ ID NO: 117; or
k. a VH-CDR1 comprising the amino acid sequence of SEQ ID NO: 121, a VH-CDR2 comprising the amino acid sequence of SEQ ID NO: 122, a VH-CDR3 comprising the amino acid sequence SEQ ID NO: 123, a VL-CDR1 comprising the amino sequence SEQ ID NO: 125, a VL-CDR2 comprising the amino acid sequence of SEQ ID NO: 126, and a VL-CDR3 comprising the amino acid sequence of SEQ ID NO: 127; or
l. a VH-CDR1 comprising the amino acid sequence of SEQ ID NO: 131, a VH-CDR2 comprising the amino acid sequence of SEQ ID NO: 132, a VH-CDR3 comprising the amino acid sequence SEQ ID NO: 133, a VL-CDR1 comprising the amino sequence SEQ ID NO: 135, a VL-CDR2 comprising the amino acid sequence of SEQ ID NO: 66, and a VL-CDR3 comprising the amino acid sequence of SEQ ID NO: 137; or
m. a VH-CDR1 comprising the amino acid sequence of SEQ ID NO: 111, a VH-CDR2 comprising the amino acid sequence of SEQ ID NO: 142, a VH-CDR3 comprising the amino acid sequence SEQ ID NO: 143, a VL-CDR1 comprising the amino sequence SEQ ID NO: 145, a VL-CDR2 comprising the amino acid sequence of SEQ ID NO: 66, and a VL-CDR3 comprising the amino acid sequence of SEQ ID NO: 147; or
n. a VH-CDR1 comprising the amino acid sequence of SEQ ID NO: 151, a VH-CDR2 comprising the amino acid sequence of SEQ ID NO: 152, a VH-CDR3 comprising the amino acid sequence SEQ ID NO: 153, a VL-CDR1 comprising the amino sequence SEQ ID NO: 155, a VL-CDR2 comprising the amino acid sequence of SEQ ID NO: 156, and a VL-CDR3 comprising the amino acid sequence of SEQ ID NO: 157; or
o. a VH-CDR1 comprising the amino acid sequence of SEQ ID NO: 161, a VH-CDR2 comprising the amino acid sequence of SEQ ID NO: 162, a VH-CDR3 comprising the amino acid sequence SEQ ID NO: 163, a VL-CDR1 comprising the amino sequence SEQ ID NO: 165, a VL-CDR2 comprising the amino acid sequence of SEQ ID NO: 166, and a VL-CDR3 comprising the amino acid sequence of SEQ ID NO: 167; or
p. a VH-CDR1 comprising the amino acid sequence of SEQ ID NO: 171, a VH-CDR2 comprising the amino acid sequence of SEQ ID NO: 172, a VH-CDR3 comprising the amino acid sequence SEQ ID NO: 173, a VL-CDR1 comprising the amino sequence SEQ ID NO: 175, a VL-CDR2 comprising the amino acid sequence of SEQ ID NO: 176, and a VL-CDR3 comprising the amino acid sequence of SEQ ID NO: 177; or
q. a VH-CDR1 comprising the amino acid sequence of SEQ ID NO: 181, a VH-CDR2 comprising the amino acid sequence of SEQ ID NO: 182, a VH-CDR3 comprising the amino acid sequence SEQ ID NO: 183, a VL-CDR1 comprising the amino sequence SEQ ID NO: 185, a VL-CDR2 comprising the amino acid sequence of SEQ ID NO: 186, and a VL-CDR3 comprising the amino acid sequence of SEQ ID NO: 187; or
r. a VH-CDR1 comprising the amino acid sequence of SEQ ID NO: 191, a VH-CDR2 comprising the amino acid sequence of SEQ ID NO: 192, a VH-CDR3 comprising the amino acid sequence SEQ ID NO: 193, a VL-CDR1 comprising the amino sequence SEQ ID NO: 195, a VL-CDR2 comprising the amino acid sequence of SEQ ID NO: 196, and a VL-CDR3 comprising the amino acid sequence of SEQ ID NO: 197; or
s. a VH-CDR1 comprising the amino acid sequence of SEQ ID NO: 201, a VH-CDR2 comprising the amino acid sequence of SEQ ID NO: 202, a VH-CDR3 comprising the amino acid sequence SEQ ID NO: 203, a VL-CDR1 comprising the amino sequence SEQ ID NO: 205, a VL-CDR2 comprising the amino acid sequence of SEQ ID NO: 206, and a VL-CDR3 comprising the amino acid sequence of SEQ ID NO: 207; or
t. a VH-CDR1 comprising the amino acid sequence of SEQ ID NO: 211, a VH-CDR2 comprising the amino acid sequence of SEQ ID NO: 212, a VH-CDR3 comprising the amino acid sequence SEQ ID NO: 213, a VL-CDR1 comprising the amino sequence SEQ ID NO: 215, a VL-CDR2 comprising the amino acid sequence of SEQ ID NO: 186, and a VL-CDR3 comprising the amino acid sequence of SEQ ID NO: 217; or
u. a VH-CDR1 comprising the amino acid sequence of SEQ ID NO: 221, a VH-CDR2 comprising the amino acid sequence of SEQ ID NO: 222, a VH-CDR3 comprising the amino acid sequence SEQ ID NO: 223, a VL-CDR1 comprising the amino sequence SEQ ID NO: 225, a VL-CDR2 comprising the amino acid sequence of SEQ ID NO: 226, and a VL-CDR3 comprising the amino acid sequence of SEQ ID NO: 227; or
v. a VH-CDR1 comprising the amino acid sequence of SEQ ID NO: 231, a VH-CDR2 comprising the amino acid sequence of SEQ ID NO: 232, a VH-CDR3 comprising the amino acid sequence SEQ ID NO: 233, a VL-CDR1 comprising the amino sequence SEQ ID NO: 235, a VL-CDR2 comprising the amino acid sequence of SEQ ID NO: 236, and a VL-CDR3 comprising the amino acid sequence of SEQ ID NO: 237; or
w. a VH-CDR1 comprising the amino acid sequence of SEQ ID NO: 241, a VH-CDR2 comprising the amino acid sequence of SEQ ID NO: 152, a VH-CDR3 comprising the amino acid sequence SEQ ID NO: 243, a VL-CDR1 comprising the amino sequence SEQ ID NO: 245, a VL-CDR2 comprising the amino acid sequence of SEQ ID NO: 246, and a VL-CDR3 comprising the amino acid sequence of SEQ ID NO: 247; or
x. a VH-CDR1 comprising the amino acid sequence of SEQ ID NO: 251, a VH-CDR2 comprising the amino acid sequence of SEQ ID NO: 252, a VH-CDR3 comprising the amino acid sequence SEQ ID NO: 253, a VL-CDR1 comprising the amino sequence SEQ ID NO: 255, a VL-CDR2 comprising the amino acid sequence of SEQ ID NO: 256, and a VL-CDR3 comprising the amino acid sequence of SEQ ID NO: 257; or
y. a VH-CDR1 comprising the amino acid sequence of SEQ ID NO: 261, a VH-CDR2 comprising the amino acid sequence of SEQ ID NO: 262, a VH-CDR3 comprising the amino acid sequence SEQ ID NO: 263, a VL-CDR1 comprising the amino sequence SEQ ID NO: 265, a VL-CDR2 comprising the amino acid sequence of SEQ ID NO: 266, and a VL-CDR3 comprising the amino acid sequence of SEQ ID NO: 267; or
z. a VH-CDR1 comprising the amino acid sequence of SEQ ID NO: 271, a VH-CDR2 comprising the amino acid sequence of SEQ ID NO: 272, a VH-CDR3 comprising the amino acid sequence SEQ ID NO: 203, a VL-CDR1 comprising the amino sequence SEQ ID NO: 275, a VL-CDR2 comprising the amino acid sequence of SEQ ID NO: 276, and a VL-CDR3 comprising the amino acid sequence of SEQ ID NO: 207; or
aa. a VH-CDR1 comprising the amino acid sequence of SEQ ID NO: 281, a VH-CDR2 comprising the amino acid sequence of SEQ ID NO: 152, a VH-CDR3 comprising the amino acid sequence SEQ ID NO: 283, a VL-CDR1 comprising the amino sequence SEQ ID NO: 285, a VL-CDR2 comprising the amino acid sequence of SEQ ID NO: 286, and a VL-CDR3 comprising the amino acid sequence of SEQ ID NO: 287; or
bb. a VH-CDR1 comprising the amino acid sequence of SEQ ID NO: 291, a VH-CDR2 comprising the amino acid sequence of SEQ ID NO: 292, a VH-CDR3 comprising the amino acid sequence SEQ ID NO: 293, a VL-CDR1 comprising the amino sequence SEQ ID NO: 295, a VL-CDR2 comprising the amino acid sequence of SEQ ID NO: 26, and a VL-CDR3 comprising the amino acid sequence of SEQ ID NO: 297; or
cc. a VH-CDR1 comprising the amino acid sequence of SEQ ID NO: 71, a VH-CDR2 comprising the amino acid sequence of SEQ ID NO: 302, a VH-CDR3 comprising the amino acid sequence SEQ ID NO: 303, a VL-CDR1 comprising the amino sequence SEQ ID NO: 305, a VL-CDR2 comprising the amino acid sequence of SEQ ID NO: 126, and a VL-CDR3 comprising the amino acid sequence of SEQ ID NO: 307; or
dd. a VH-CDR1 comprising the amino acid sequence of SEQ ID NO: 161, a VH-CDR2 comprising the amino acid sequence of SEQ ID NO: 312, a VH-CDR3 comprising the amino acid sequence RDY (Arg-Asp-Tyr), a VL-CDR1 comprising the amino sequence SEQ ID NO: 315, a VL-CDR2 comprising the amino acid sequence of SEQ ID NO: 186, and a VL-CDR3 comprising the amino acid sequence of SEQ ID NO: 317; or
ee. a VH-CDR1 comprising the amino acid sequence of SEQ ID NO: 161, a VH-CDR2 comprising the amino acid sequence of SEQ ID NO: 322, a VH-CDR3 comprising the amino acid sequence SEQ ID NO: 323, a VL-CDR1 comprising the amino sequence SEQ ID NO: 205, a VL-CDR2 comprising the amino acid sequence of SEQ ID NO: 326, and a VL-CDR3 comprising the amino acid sequence of SEQ ID NO: 327; or
ff. a VH-CDR1 comprising the amino acid sequence of SEQ ID NO: 331, a VH-CDR2 comprising the amino acid sequence of SEQ ID NO: 332, a VH-CDR3 comprising the amino acid sequence SEQ ID NO: 333, a VL-CDR1 comprising the amino sequence SEQ ID NO: 335, a VL-CDR2 comprising the amino acid sequence of SEQ ID NO: 336, and a VL-CDR3 comprising the amino acid sequence of SEQ ID NO: 337; or
gg. a VH-CDR1 comprising the amino acid sequence of SEQ ID NO: 161, a VH-CDR2 comprising the amino acid sequence of SEQ ID NO: 342, a VH-CDR3 comprising the amino acid sequence SEQ ID NO: 323, a VL-CDR1 comprising the amino sequence SEQ ID NO: 205, a VL-CDR2 comprising the amino acid sequence of SEQ ID NO: 326, and a VL-CDR3 comprising the amino acid sequence of SEQ ID NO: 347; or
hh. a VH-CDR1 comprising the amino acid sequence of SEQ ID NO: 331, a VH-CDR2 comprising the amino acid sequence of SEQ ID NO: 352, a VH-CDR3 comprising the amino acid sequence SEQ ID NO: 353, a VL-CDR1 comprising the amino sequence SEQ ID NO: 205, a VL-CDR2 comprising the amino acid sequence of SEQ ID NO: 336, and a VL-CDR3 comprising the amino acid sequence of SEQ ID NO: 337; or
ii. a VH-CDR1 comprising the amino acid sequence of SEQ ID NO: 361, a VH-CDR2 comprising the amino acid sequence of SEQ ID NO: 362, a VH-CDR3 comprising the amino acid sequence RDY (Arg-Asp-Tyr), a VL-CDR1 comprising the amino sequence SEQ ID NO: 315, a VL-CDR2 comprising the amino acid sequence of SEQ ID NO: 186, and a VL-CDR3 comprising the amino acid sequence of SEQ ID NO: 367; or
jj. a VH-CDR1 comprising the amino acid sequence of SEQ ID NO: 371, a VH-CDR2 comprising the amino acid sequence of SEQ ID NO: 372, a VH-CDR3 comprising the amino acid sequence SEQ ID NO: 373, a VL-CDR1 comprising the amino sequence SEQ ID NO: 375, a VL-CDR2 comprising the amino acid sequence of SEQ ID NO: 376, and a VL-CDR3 comprising the amino acid sequence of SEQ ID NO: 377; or
kk. a VH-CDR1 comprising the amino acid sequence of SEQ ID NO: 381, a VH-CDR2 comprising the amino acid sequence of SEQ ID NO: 382, a VH-CDR3 comprising the amino acid sequence SEQ ID NO: 383, a VL-CDR1 comprising the amino sequence SEQ ID NO: 385, a VL-CDR2 comprising the amino acid sequence of SEQ ID NO: 386, and a VL-CDR3 comprising the amino acid sequence of SEQ ID NO: 387; or
ll. a VH-CDR1 comprising the amino acid sequence of SEQ ID NO: 271, a VH-CDR2 comprising the amino acid sequence of SEQ ID NO: 392, a VH-CDR3 comprising the amino acid sequence SEQ ID NO: 393, a VL-CDR1 comprising the amino sequence SEQ ID NO: 335, a VL-CDR2 comprising the amino acid sequence of SEQ ID NO: 276, and a VL-CDR3 comprising the amino acid sequence of SEQ ID NO: 207.

In one embodiment, the ASC binding molecule of the invention comprises:
a. a Heavy Chain Variable Region (VH) comprising the amino acid sequence of SEQ ID NO: 10 or a Heavy Chain Variable Region (VH) having at least 92%, 93%, 94%, 95%, 96%, 97%, 98% or 99% sequence identity to the amino acid sequence of SEQ ID NO: 10; and a Light Chain Variable Region (VL) comprising the amino acid sequence of SEQ ID NO: 14 or a Light Chain Variable Region (VL) having at least 98% or 99% sequence identity to the amino acid sequence of SEQ ID NO: 14; or
b. a Heavy Chain Variable Region (VH) comprising the amino acid sequence of SEQ ID NO: 20 or a Heavy Chain Variable Region (VH) having at least 90%, 91%, 92%, 93%, 94%, 95%, 96%, 97%, 98% or 99% sequence identity to the amino acid sequence of SEQ ID NO: 20; and a Light Chain Variable Region (VL) comprising the amino acid sequence of SEQ ID NO: 24 or a Light Chain Variable Region (VL) having at least 95%, 96%, 97%, 98% or 99% sequence identity to the amino acid sequence of SEQ ID NO: 24; or
c. a Heavy Chain Variable Region (VH) comprising the amino acid sequence of SEQ ID NO: 30 or a Heavy Chain Variable Region (VH) having at least 85%, 86%, 87%, 88%, 89%, 90%, 91%, 92%, 93%, 94%, 95%, 96%, 97%, 98% or 99% sequence identity to the amino acid sequence of SEQ ID NO: 30; and a Light Chain Variable Region (VL) comprising the amino acid sequence of SEQ ID NO: 34 or a Light Chain Variable Region (VL) having at least 93%, 94%, 95%, 96%, 97%, 98% or 99% sequence identity to the amino acid sequence of SEQ ID NO: 34; or
d. a Heavy Chain Variable Region (VH) comprising the amino acid sequence of SEQ ID NO: 40 or a Heavy Chain Variable Region (VH) having at least 86%, 87%, 88%, 89%, 90%, 91%, 92%, 93%, 94%, 95%, 96%, 97%, 98% or 99% sequence identity to the amino acid sequence of SEQ ID NO: 40; and a Light Chain Variable Region (VL) comprising the amino acid sequence of SEQ ID NO: 44 or a Light Chain Variable Region (VL) having at least 96%, 97%, 98% or 99% sequence identity to the amino acid sequence of SEQ ID NO: 44; or
e. a Heavy Chain Variable Region (VH) comprising the amino acid sequence of SEQ ID NO: 50 or a Heavy Chain Variable Region (VH) having at least 94%, 95%, 96%, 97%, 98% or 99% sequence identity to the amino acid sequence of SEQ ID NO: 50; and a Light Chain Variable Region (VL) comprising the amino acid sequence of SEQ ID NO: 54 or a Light Chain Variable Region (VL) having at least 96%, 97%, 98% or 99% sequence identity to the amino acid sequence of SEQ ID NO: 54; or
f. a Heavy Chain Variable Region (VH) comprising the amino acid sequence of SEQ ID NO: 60 or a Heavy Chain Variable Region (VH) having at least 85%, 86%, 87%, 88%, 95%, 96%, 97%, 98% or 99% sequence identity to the amino acid sequence of SEQ ID NO: 60; and a Light Chain Variable Region (VL) comprising the amino acid sequence of SEQ ID NO: 64; or
g. a Heavy Chain Variable Region (VH) comprising the amino acid sequence of SEQ ID NO: 70 or a Heavy Chain Variable Region (VH) having at least 90%, 91%, 92%, 93%, 94%, 95%, 96%, 97%, 98% or 99% sequence identity to the amino acid sequence of SEQ ID NO: 70; and a Light Chain Variable Region (VL) comprising the amino acid sequence of SEQ ID NO: 74 or a Light Chain Variable Region (VL) having at least 97%, 98% or 99% sequence identity to the amino acid sequence of SEQ ID NO: 74; or
h. a Heavy Chain Variable Region (VH) comprising the amino acid sequence of SEQ ID NO: 80 or a Heavy Chain Variable Region (VH) having at least 93%, 94%, 95%, 96%, 97%, 98% or 99% sequence identity to the amino acid sequence of SEQ ID NO: 80; and a Light Chain Variable Region (VL) comprising the amino acid sequence of SEQ ID NO: 84 or a Light Chain Variable Region (VL) having at least 95%, 96%, 97%, 98% or 99% sequence identity to the amino acid sequence of SEQ ID NO: 84; or
i. a Heavy Chain Variable Region (VH) comprising the amino acid sequence of SEQ ID NO: 90 or a Heavy Chain Variable Region (VH) having at least 93%, 94%, 95%, 96%, 97%, 98% or 99% sequence identity to the amino acid sequence of SEQ ID NO: 90; and a Light Chain Variable Region (VL) comprising the amino acid sequence of SEQ ID NO: 94 or a Light Chain Variable Region (VL) having at least 93%, 94%, 95%, 96%, 97%, 98% or 99% sequence identity to the amino acid sequence of SEQ ID NO: 94; or
j. a Heavy Chain Variable Region (VH) comprising the amino acid sequence of SEQ ID NO: 110 or a Heavy Chain Variable Region (VH) having at least 88%, 89%, 90%, 91%, 92%, 93%, 94%, 95%, 96%, 97%, 98% or 99% sequence identity to the amino acid sequence of SEQ ID NO: 110; and a Light Chain Variable Region (VL) comprising the amino acid sequence of SEQ ID NO: 114 or a Light Chain Variable Region (VL) having at least 98% or 99% sequence identity to the amino acid sequence of SEQ ID NO: 114; or
k. a Heavy Chain Variable Region (VH) comprising the amino acid sequence of SEQ ID NO: 120 or a Heavy Chain Variable Region (VH) having at least 92%, 93%, 94%, 95%, 96%, 97%, 98% or 99% sequence identity to the amino acid sequence of SEQ ID NO: 120; and a Light Chain Variable Region (VL) comprising the amino acid sequence of SEQ ID NO: 124 or a Light Chain Variable Region (VL) having at least 98% or 99% sequence identity to the amino acid sequence of SEQ ID NO: 124; or
l. a Heavy Chain Variable Region (VH) comprising the amino acid sequence of SEQ ID NO: 130 or a Heavy Chain Variable Region (VH) having at least 87%, 88%, 89%, 90%, 91%, 92%, 93%, 94%, 95%, 96%, 97%, 98% or 99% sequence identity to the amino acid sequence of SEQ ID NO: 130; and a Light Chain Variable Region (VL) comprising the amino acid sequence of SEQ ID NO: 134 or a Light Chain Variable Region (VL) having at least 98% or 99% sequence identity to the amino acid sequence of SEQ ID NO: 134; or
m. a Heavy Chain Variable Region (VH) comprising the amino acid sequence of SEQ ID NO: 140 or a Heavy Chain Variable Region (VH) having at least 94%, 95%, 96%, 97%, 98% or 99% sequence identity to the amino acid sequence of SEQ ID NO: 140; and a Light Chain Variable Region (VL) comprising the amino acid sequence of SEQ ID NO: 144 or a Light Chain Variable Region (VL) having at least 98% or 99% sequence identity to the amino acid sequence of SEQ ID NO: 144; or
n. a Heavy Chain Variable Region (VH) comprising the amino acid sequence of SEQ ID NO: 150 or a Heavy Chain Variable Region (VH) having at least 90%, 91%, 92%, 93%, 94%, 95%, 96%, 97%, 98% or 99% sequence identity to the amino acid sequence of SEQ ID NO: 150; and a Light Chain Variable Region (VL) comprising the amino acid sequence of SEQ ID NO: 154 or a Light Chain Variable Region (VL) having at least 93%, 94%, 95%, 96%, 97%, 98% or 99% sequence identity to the amino acid sequence of SEQ ID NO: 154; or
o. a Heavy Chain Variable Region (VH) comprising the amino acid sequence of SEQ ID NO: 160 or a Heavy Chain Variable Region (VH) having at least 88%, 89%, 90%, 91%, 92%, 93%, 94%, 95%, 96%, 97%, 98% or 99% sequence identity to the amino acid sequence of SEQ ID NO: 160; and a Light Chain Variable Region (VL) comprising the amino acid sequence of SEQ ID NO: 164 or a Light Chain Variable Region (VL) having at least 96%, 97%, 98% or 99% sequence identity to the amino acid sequence of SEQ ID NO: 164; or
p. a Heavy Chain Variable Region (VH) comprising the amino acid sequence of SEQ ID NO: 170 or a Heavy Chain Variable Region (VH) having at least 85%, 86%, 87%, 88%, 89%, 90%, 91%, 92%, 93%, 94%, 95%, 96%, 97%, 98% or 99% sequence identity to the amino acid sequence of SEQ ID NO: 170; and a Light Chain Variable Region (VL) comprising the amino acid sequence of SEQ ID NO: 174 or a Light Chain Variable Region (VL) having at least 93%, 94%, 95%, 96%, 97%, 98% or 99% sequence identity to the amino acid sequence of SEQ ID NO: 174; or
q. a Heavy Chain Variable Region (VH) comprising the amino acid sequence of SEQ ID NO: 180 or a Heavy Chain Variable Region (VH) having at least 94%, 95%, 96%, 97%, 98% or 99% sequence identity to the amino acid sequence of SEQ ID NO: 180; and a Light Chain Variable Region (VL) comprising the amino acid sequence of SEQ ID NO: 184 or a Light Chain Variable Region (VL) having at least 97%, 98% or 99% sequence identity to the amino acid sequence of SEQ ID NO: 184; or
r. a Heavy Chain Variable Region (VH) comprising the amino acid sequence of SEQ ID NO: 190 or a Heavy Chain Variable Region (VH) having at least 87%, 88%, 89%, 90%, 91%, 92%, 93%, 94%, 95%, 96%, 97%, 98% or 99% sequence identity to the amino acid sequence of SEQ ID NO: 190; and a Light Chain Variable Region (VL) comprising the amino acid sequence of SEQ ID NO: 194; or
s. a Heavy Chain Variable Region (VH) comprising the amino acid sequence of SEQ ID NO: 200 or a Heavy Chain Variable Region (VH) having at least 93%, 94%, 95%, 96%, 97%, 98% or 99% sequence identity to the amino acid sequence of SEQ ID NO: 200; and a Light Chain Variable Region (VL) comprising the amino acid sequence of SEQ ID NO: 204 or a Light Chain Variable Region (VL) having at least 99% sequence identity to the amino acid sequence of SEQ ID NO: 204; or
t. a Heavy Chain Variable Region (VH) comprising the amino acid sequence of SEQ ID NO: 210 or a Heavy Chain Variable Region (VH) having at least 92%, 93%, 94%, 95%, 96%, 97%, 98% or 99% sequence identity to the amino acid sequence of SEQ ID NO: 210; and a Light Chain Variable Region (VL) comprising the amino acid sequence of SEQ ID NO: 214 or a Light Chain Variable Region (VL) having at least 96%, 97%, 98% or 99% sequence identity to the amino acid sequence of SEQ ID NO: 214; or
u. a Heavy Chain Variable Region (VH) comprising the amino acid sequence of SEQ ID NO: 220 or a Heavy Chain Variable Region (VH) having at least 84%, 85%, 86%, 87%, 88%, 89%, 90%, 91%, 92%, 93%, 94%, 95%, 96%, 97%, 98% or 99% sequence identity to the amino acid sequence of SEQ ID NO: 220; and a Light Chain Variable Region (VL) comprising the amino acid sequence of SEQ ID NO: 224 or a Light Chain Variable Region (VL) having at least 93%, 94%, 95%, 96%, 97%, 98% or 99% sequence identity to the amino acid sequence of SEQ ID NO: 224; or
v. a Heavy Chain Variable Region (VH) comprising the amino acid sequence of SEQ ID NO: 230 or a Heavy Chain Variable Region (VH) having at least 84%, 85%, 86%, 87%, 88%, 89%, 90%, 91%, 92%, 93%, 94%, 95%, 96%, 97%, 98% or 99% sequence identity to the amino acid sequence of SEQ ID NO: 230; and a Light Chain Variable Region (VL) comprising the amino acid sequence of SEQ ID NO: 234 or a Light Chain Variable Region (VL) having at least 92%, 93%, 94%, 95%, 96%, 97%, 98% or 99% sequence identity to the amino acid sequence of SEQ ID NO: 234; or
w. a Heavy Chain Variable Region (VH) comprising the amino acid sequence of SEQ ID NO: 240 or a Heavy Chain Variable Region (VH) having at least 88%, 89%, 90%, 91%, 92%, 93%, 94%, 95%, 96%, 97%, 98% or 99% sequence identity to the amino acid sequence of SEQ ID NO: 240; and a Light Chain Variable Region (VL) comprising the amino acid sequence of SEQ ID NO: 244 or a Light Chain Variable Region (VL) having at least 97%, 98% or 99% sequence identity to the amino acid sequence of SEQ ID NO: 244; or
x. a Heavy Chain Variable Region (VH) comprising the amino acid sequence of SEQ ID NO: 250 or a Heavy Chain Variable Region (VH) having at least 89%, 90%, 91%, 92%, 93%, 94%, 95%, 96%, 97%, 98% or 99% sequence identity to the amino acid sequence of SEQ ID NO: 250; and a Light Chain Variable Region (VL) comprising the amino acid sequence of SEQ ID NO: 254 or a Light Chain Variable Region (VL) having at least 97%, 98% or 99% sequence identity to the amino acid sequence of SEQ ID NO: 254; or
y. a Heavy Chain Variable Region (VH) comprising the amino acid sequence of SEQ ID NO: 260 or a Heavy Chain Variable Region (VH) having at least 90%, 91%, 92%, 93%, 94%, 95%, 96%, 97%, 98% or 99% sequence identity to the amino acid sequence of SEQ ID NO: 260; and a Light Chain Variable Region (VL) comprising the amino acid sequence of SEQ ID NO: 264 or a Light Chain Variable Region (VL) having at least 97%, 98% or 99% sequence identity to the amino acid sequence of SEQ ID NO: 264; or
z. a Heavy Chain Variable Region (VH) comprising the amino acid sequence of SEQ ID NO: 270 or a Heavy Chain Variable Region (VH) having at least 91%, 92%, 93%, 94%, 95%, 96%, 97%, 98% or 99% sequence identity to the amino acid sequence of SEQ ID NO: 270; and a Light Chain Variable Region (VL) comprising the amino acid sequence of SEQ ID NO: 274 or a Light Chain Variable Region (VL) having at least or 99% sequence identity to the amino acid sequence of SEQ ID NO: 274; or
aa. a Heavy Chain Variable Region (VH) comprising the amino acid sequence of SEQ ID NO: 280 or a Heavy Chain Variable Region (VH) having at least 87%, 88%, 89%, 90%, 91%, 92%, 93%, 94%, 95%, 96%, 97%, 98% or 99% sequence identity to the amino acid sequence of SEQ ID NO: 280; and a Light Chain Variable Region (VL) comprising the amino acid sequence of SEQ ID NO: 284 or a Light Chain Variable Region (VL) having at least 98% or 99% sequence identity to the amino acid sequence of SEQ ID NO: 284; or
bb. a Heavy Chain Variable Region (VH) comprising the amino acid sequence of SEQ ID NO: 290 or a Heavy Chain Variable Region (VH) having at least 92%, 93%, 94%, 95%, 96%, 97%, 98% or 99% sequence identity to the amino acid sequence of SEQ ID NO: 290; and a Light Chain Variable Region (VL) comprising the amino acid sequence of SEQ ID NO: 294 or a Light Chain Variable Region (VL) having at least 99% sequence identity to the amino acid sequence of SEQ ID NO: 294; or
cc. a Heavy Chain Variable Region (VH) comprising the amino acid sequence of SEQ ID NO: 300 or a Heavy Chain Variable Region (VH) having at least 95%, 96%, 97%, 98% or 99% sequence identity to the amino acid sequence of SEQ ID NO: 300; and a Light Chain Variable Region (VL) comprising the amino acid sequence of SEQ ID NO: 304 or a Light Chain Variable Region (VL) having at least 98% or 99% sequence identity to the amino acid sequence of SEQ ID NO: 304; or
dd. a Heavy Chain Variable Region (VH) comprising the amino acid sequence of SEQ ID NO: 310 or a Heavy Chain Variable Region (VH) having at least 95%, 96%, 97%, 98% or 99% sequence identity to the amino acid sequence of SEQ ID NO: 310; and a Light Chain Variable Region (VL) comprising the amino acid sequence of SEQ ID NO: 314 or a Light Chain Variable Region (VL) having at least 97%, 98% or 99% sequence identity to the amino acid sequence of SEQ ID NO: 314; or
ee. a Heavy Chain Variable Region (VH) comprising the amino acid sequence of SEQ ID NO: 320 or a Heavy Chain Variable Region (VH) having at least 93%, 94%, 95%, 96%, 97%, 98% or 99% sequence identity to the amino acid sequence of SEQ ID NO: 320; and a Light Chain Variable Region (VL) comprising the amino acid sequence of SEQ ID NO: 324 or a Light Chain Variable Region (VL) having at least 94%, 95%, 96%, 97%, 98% or 99% sequence identity to the amino acid sequence of SEQ ID NO: 324; or
ff. a Heavy Chain Variable Region (VH) comprising the amino acid sequence of SEQ ID NO: 330 or a Heavy Chain Variable Region (VH) having at least 87%, 88%, 89%, 90%, 91%, 92%, 93%, 94%, 95%, 96%, 97%, 98% or 99% sequence identity to the amino acid sequence of SEQ ID NO: 330; and a Light Chain Variable Region (VL) comprising the amino acid sequence of SEQ ID NO: 334 or a Light Chain Variable Region (VL) having at least 96%, 97%, 98% or 99% sequence identity to the amino acid sequence of SEQ ID NO: 334; or
gg. a Heavy Chain Variable Region (VH) comprising the amino acid sequence of SEQ ID NO: 340 or a Heavy Chain Variable Region (VH) having at least 89%, 90%, 91%, 92%, 93%, 94%, 95%, 96%, 97%, 98% or 99% sequence identity to the amino acid sequence of SEQ ID NO: 340; and a Light Chain Variable Region (VL) comprising the amino acid sequence of SEQ ID NO: 344 or a Light Chain Variable Region (VL) having at least 89%, 90%, 91%, 92%, 93%, 94%, 95%, 96%, 97%, 98% or 99% sequence identity to the amino acid sequence of SEQ ID NO: 344; or
hh. a Heavy Chain Variable Region (VH) comprising the amino acid sequence of SEQ ID NO: 350 or a Heavy Chain Variable Region (VH) having at least 89%, 90%, 91%, 92%, 93%, 94%, 95%, 96%, 97%, 98% or 99% sequence identity to the amino acid sequence of SEQ ID NO: 350; and a Light Chain Variable Region (VL) comprising the amino acid sequence of SEQ ID NO: 354 or a Light Chain Variable Region (VL) having at least 96%, 97%, 98% or 99% sequence identity to the amino acid sequence of SEQ ID NO: 354; or
ii. a Heavy Chain Variable Region (VH) comprising the amino acid sequence of SEQ ID NO: 360 or a Heavy Chain Variable Region (VH) having at least 92%, 93%, 94%, 95%, 96%, 97%, 98% or 99% sequence identity to the amino acid sequence of SEQ ID NO: 360; and a Light Chain Variable Region (VL) comprising the amino acid sequence of SEQ ID NO: 364 or a Light Chain Variable Region (VL) having at least 98% or 99% sequence identity to the amino acid sequence of SEQ ID NO: 364; or
jj. a Heavy Chain Variable Region (VH) comprising the amino acid sequence of SEQ ID NO: 370 or a Heavy Chain Variable Region (VH) having at least 85%, 86%, 87%, 88%, 89%, 90%, 91%, 92%, 93%, 94%, 95%, 96%, 97%, 98% or 99% sequence identity to the amino acid sequence of SEQ ID NO: 370; and a Light Chain Variable Region (VL) comprising the amino acid sequence of SEQ ID NO: 374 or a Light Chain Variable Region (VL) having at least 94%, 95%, 96%, 97%, 98% or 99% sequence identity to the amino acid sequence of SEQ ID NO: 374; or
kk. a Heavy Chain Variable Region (VH) comprising the amino acid sequence of SEQ ID NO: 380 or a Heavy Chain Variable Region (VH) having at least 86%, 87%, 88%, 89%, 90%, 91%, 92%, 93%, 94%, 95%, 96%, 97%, 98% or 99% sequence identity to the amino acid sequence of SEQ ID NO: 380; and a Light Chain Variable Region (VL) comprising the amino acid sequence of SEQ ID NO: 384 or a Light Chain Variable Region (VL) having at least 93%, 94%, 95%, 96%, 97%, 98% or 99% sequence identity to the amino acid sequence of SEQ ID NO: 384; or
ll. a Heavy Chain Variable Region (VH) comprising the amino acid sequence of SEQ ID NO: 390 or a Heavy Chain Variable Region (VH) having at least 95%, 96%, 97%, 98% or 99% sequence identity to the amino acid sequence of SEQ ID NO: 390; and a Light Chain Variable Region (VL) comprising the amino acid sequence of SEQ ID NO: 394.

In one embodiment, the ASC binding molecule of the invention comprises:
a. a Heavy Chain Variable Region (VH) comprising the amino acid sequence of SEQ ID NO: 10 and a Light Chain Variable Region (VL) comprising the amino acid sequence of SEQ ID NO: 14; or
b. a Heavy Chain Variable Region (VH) comprising the amino acid sequence of SEQ ID NO: 20; and a Light Chain Variable Region (VL) comprising the amino acid sequence of SEQ ID NO: 24; or
c. a Heavy Chain Variable Region (VH) comprising the amino acid sequence of SEQ ID NO: 30 and a Light Chain Variable Region (VL) comprising the amino acid sequence of SEQ ID NO: 14; or
d. a Heavy Chain Variable Region (VH) comprising the amino acid sequence of SEQ ID NO: 40 and a Light Chain Variable Region (VL) comprising the amino acid sequence of SEQ ID NO: 44; or
e. a Heavy Chain Variable Region (VH) comprising the amino acid sequence of SEQ ID NO: 50; and a Light Chain Variable Region (VL) comprising the amino acid sequence of SEQ ID NO: 54; or
f. a Heavy Chain Variable Region (VH) comprising the amino acid sequence of SEQ ID NO: 60 and a Light Chain Variable Region (VL) comprising the amino acid sequence of SEQ ID NO: 64; or
g. a Heavy Chain Variable Region (VH) comprising the amino acid sequence of SEQ ID NO: 70 and a Light Chain Variable Region (VL) comprising the amino acid sequence of SEQ ID NO: 74; or
h. a Heavy Chain Variable Region (VH) comprising the amino acid sequence of SEQ ID NO: 80 and a Light Chain Variable Region (VL) comprising the amino acid sequence of SEQ ID NO: 84; or
i. a Heavy Chain Variable Region (VH) comprising the amino acid sequence of SEQ ID NO: 90 and a Light Chain Variable Region (VL) comprising the amino acid sequence of SEQ ID NO: 94; or
j. a Heavy Chain Variable Region (VH) comprising the amino acid sequence of SEQ ID NO: 110 and a Light Chain Variable Region (VL) comprising the amino acid sequence of SEQ ID NO: 114; or
k. a Heavy Chain Variable Region (VH) comprising the amino acid sequence of SEQ ID NO: 120 and a Light Chain Variable Region (VL) comprising the amino acid sequence of SEQ ID NO: 124; or
l. a Heavy Chain Variable Region (VH) comprising the amino acid sequence of SEQ ID NO: 130 and a Light Chain Variable Region (VL) comprising the amino acid sequence of SEQ ID NO: 134; or
m. a Heavy Chain Variable Region (VH) comprising the amino acid sequence of SEQ ID NO: 140 and a Light Chain Variable Region (VL) comprising the amino acid sequence of SEQ ID NO: 144; or
n. a Heavy Chain Variable Region (VH) comprising the amino acid sequence of SEQ ID NO: 150 and a Light Chain Variable Region (VL) comprising the amino acid sequence of SEQ ID NO: 154; or
o. a Heavy Chain Variable Region (VH) comprising the amino acid sequence of SEQ ID NO: 160 and a Light Chain Variable Region (VL) comprising the amino acid sequence of SEQ ID NO: 164; or
p. a Heavy Chain Variable Region (VH) comprising the amino acid sequence of SEQ ID NO: 170 and a Light Chain Variable Region (VL) comprising the amino acid sequence of SEQ ID NO: 174; or
q. a Heavy Chain Variable Region (VH) comprising the amino acid sequence of SEQ ID NO: 180 and a Light Chain Variable Region (VL) comprising the amino acid sequence of SEQ ID NO: 184; or
r. a Heavy Chain Variable Region (VH) comprising the amino acid sequence of SEQ ID NO: 190 and a Light Chain Variable Region (VL) comprising the amino acid sequence of SEQ ID NO: 194; or
s. a Heavy Chain Variable Region (VH) comprising the amino acid sequence of SEQ ID NO: 200 and a Light Chain Variable Region (VL) comprising the amino acid sequence of SEQ ID NO: 204; or
t. a Heavy Chain Variable Region (VH) comprising the amino acid sequence of SEQ ID NO: 210 and a Light Chain Variable Region (VL) comprising the amino acid sequence of SEQ ID NO: 214; or
u. a Heavy Chain Variable Region (VH) comprising the amino acid sequence of SEQ ID NO: 220 and a Light Chain Variable Region (VL) comprising the amino acid sequence of SEQ ID NO: 224; or
v. a Heavy Chain Variable Region (VH) comprising the amino acid sequence of SEQ ID NO: 230 and a Light Chain Variable Region (VL) comprising the amino acid sequence of SEQ ID NO: 234; or
w. a Heavy Chain Variable Region (VH) comprising the amino acid sequence of SEQ ID NO: 240 and a Light Chain Variable Region (VL) comprising the amino acid sequence of SEQ ID NO: 244; or
x. a Heavy Chain Variable Region (VH) comprising the amino acid sequence of SEQ ID NO: 250 and a Light Chain Variable Region (VL) comprising the amino acid sequence of SEQ ID NO: 254; or
y. a Heavy Chain Variable Region (VH) comprising the amino acid sequence of SEQ ID NO: 260 and a Light Chain Variable Region (VL) comprising the amino acid sequence of SEQ ID NO: 264; or
z. a Heavy Chain Variable Region (VH) comprising the amino acid sequence of SEQ ID NO: 270 and a Light Chain Variable Region (VL) comprising the amino acid sequence of SEQ ID NO: 274; or
aa. a Heavy Chain Variable Region (VH) comprising the amino acid sequence of SEQ ID NO: 280 and a Light Chain Variable Region (VL) comprising the amino acid sequence of SEQ ID NO: 284; or
bb. a Heavy Chain Variable Region (VH) comprising the amino acid sequence of SEQ ID NO: 290; and a Light Chain Variable Region (VL) comprising the amino acid sequence of SEQ ID NO: 294; or
cc. a Heavy Chain Variable Region (VH) comprising the amino acid sequence of SEQ ID NO: 300 and a Light Chain Variable Region (VL) comprising the amino acid sequence of SEQ ID NO:304; or
dd. a Heavy Chain Variable Region (VH) comprising the amino acid sequence of SEQ ID NO: 310; and a Light Chain Variable Region (VL) comprising the amino acid sequence of SEQ ID NO: 314; or
ee. a Heavy Chain Variable Region (VH) comprising the amino acid sequence of SEQ ID NO: 320 and a Light Chain Variable Region (VL) comprising the amino acid sequence of SEQ ID NO: 324; or
ff. a Heavy Chain Variable Region (VH) comprising the amino acid sequence of SEQ ID NO: 330; and a Light Chain Variable Region (VL) comprising the amino acid sequence of SEQ ID NO: 334; or
gg. a Heavy Chain Variable Region (VH) comprising the amino acid sequence of SEQ ID NO: 340 and a Light Chain Variable Region (VL) comprising the amino acid sequence of SEQ ID NO: 344; or
hh. a Heavy Chain Variable Region (VH) comprising the amino acid sequence of SEQ ID NO: 350 and a Light Chain Variable Region (VL) comprising the amino acid sequence of SEQ ID NO: 354; or
ii. a Heavy Chain Variable Region (VH) comprising the amino acid sequence of SEQ ID NO: 360 and a Light Chain Variable Region (VL) comprising the amino acid sequence of SEQ ID NO: 364; or
jj. a Heavy Chain Variable Region (VH) comprising the amino acid sequence of SEQ ID NO: 370 and a Light Chain Variable Region (VL) comprising the amino acid sequence of SEQ ID NO: 374; or
kk. a Heavy Chain Variable Region (VH) comprising the amino acid sequence of SEQ ID NO: 380 and a Light Chain Variable Region (VL) comprising the amino acid sequence of SEQ ID NO: 384; or
ll. a Heavy Chain Variable Region (VH) comprising the amino acid sequence of SEQ ID NO: 390 and a Light Chain Variable Region (VL) comprising the amino acid sequence of SEQ ID NO: 394.

In one embodiment, the ASC binding molecule is an anti-ASC antibody or an antigen-binding fragment thereof. In one embodiment, the ASC binding molecule, preferably an anti-ASC antibody or an antigen-binding fragment thereof, is a monoclonal antibody or an antigen-binding fragment thereof. In one embodiment, the anti-ASC antibody or an antigen-binding fragment thereof of the invention is an IgA, IgD, IgE, IgM, IgG1, IgG2, IgG2a, IgG2b, IgG3 or IgG4 antibody or antigen-binding fragment thereof, preferably human IgA, IgD, IgE, IgM, IgG1, IgG2, IgG2a, IgG2b, IgG3 or IgG4.

In one embodiment, the ASC binding molecule is an antibody or an antibody-binding fragment thereof comprising the sequence defined by ACI-8016-416E6G4-AB1, ACI-8016-402H11C9-Ab1, ACI-8016-203B12C3-AB1, ACI-8016-421B10C12D2-AB1, ACI-8016-417E12A8-AB1, ACI-8016-413G10A5-AB1, ACI-8016-407E10A9-AB1, ACI-8016-203G8B10-AB1, ACI-8016-401H9B7-AB1, ACI-8016-1112B3D7-AB1, ACI-8018-2221B7F1-AB1, ACI-8019-2314F6H11-AB1, ACI-8016-207E8B2-AB1, ACI-8016-2A1B12-AB1, ACI-8016-17H1G2-AB1, ACI-8016-18F4C12-AB1, ACI-8016-23E5F7-AB1, ACI-8016-23E5F7-AB2, ACI-8016-26A1G2-AB1, ACI-8016-32B6C7-AB1, ACI-8016-22D3A6-AB1, ACI-8016-31F10C5-AB1, ACI-8016-19E6D4-AB1, ACI-8016-3E6B11-AB1, ACI-8016-11A3F3-AB1, ACI-8016-14G5B8-AB1, ACI-8016-22A10F8-AB1, ACI-8016-27A1G4-AB1, ACI-8016-29C5E11-AB1, ACI-8016-7G3B5-AB1, ACI-8016-2504F3D9-AB1, ACI-8016-2516A8C6-AB1, ACI-8016-2602H6F10-AB1, ACI-8016-2609F4A9-AB1, ACI-8016-2610H7D3-AB1, ACI-8016-2614C3B2-AB1, ACI-8016-2617C3A8-AB1, ACI-8016-2622E12F11-AB1, ACI-8016-2626B9D3-AB1, or ACI-8016-2629E8D1-AB1 as set forth in Table 17.

In one embodiment the ASC binding molecule may comprise:
a. VH-CDR1 comprising the amino acid sequence of SEQ ID NO: 201, a VH-CDR2 comprising the amino acid sequence of SEQ ID NO: 202; and a VH-CDR3 comprising the amino acid sequence SEQ ID NO: 203, a VL-CDR1 comprising the amino sequence SEQ ID NO: 205; a VL-CDR2 comprising the amino sequence SEQ ID NO: 206, and a VL-CDR3 comprising the amino sequence SEQ ID NO: 207; or
b. a VH-CDR1 comprising the amino acid sequence of SEQ ID NO: 201, a VH-CDR2 comprising the amino acid sequence of SEQ ID NO: 412 and a VH-CDR3 comprising the amino acid sequence SEQ ID NO: 203; a VL-CDR1 comprising the amino sequence SEQ ID NO: 205; a VL-CDR2 comprising the amino sequence SEQ ID NO: 206, and a VL-CDR3 comprising the amino sequence SEQ ID NO: 207; or
c. a VH-CDR1 comprising the amino acid sequence of SEQ ID NO: 201, a VH-CDR2 comprising the amino acid sequence of SEQ ID NO: 412 and a VH-CDR3 comprising the amino acid sequence SEQ ID NO: 203; a VL-CDR1 comprising the amino sequence SEQ ID NO: 435; a VL-CDR2 comprising the amino sequence SEQ ID NO: 206, and a VL-CDR3 comprising the amino sequence SEQ ID NO: 207; or
d. a VH-CDR1 comprising the amino acid sequence of SEQ ID NO: 201, a VH-CDR2 comprising the amino acid sequence of SEQ ID NO: 462 and a VH-CDR3 comprising the amino acid sequence SEQ ID NO: 203; a VL-CDR1 comprising the amino sequence SEQ ID NO: 205; a VL-CDR2 comprising the amino sequence SEQ ID NO: 206, and a VL-CDR3 comprising the amino sequence SEQ ID NO: 207; or
e. a VH-CDR1 comprising the amino acid sequence of SEQ ID NO: 201, a VH-CDR2 comprising the amino acid sequence of SEQ ID NO: 462 and a VH-CDR3 comprising the amino acid sequence SEQ ID NO: 203; a VL-CDR1 comprising the amino sequence SEQ ID NO: 435; a VL-CDR2 comprising the amino sequence SEQ ID NO: 206, and a VL-CDR3 comprising the amino sequence SEQ ID NO: 207; or
f. a VH-CDR1 comprising the amino acid sequence of SEQ ID NO: 201, a VH-CDR2 comprising the amino acid sequence of SEQ ID NO: 472 and a VH-CDR3 comprising the amino acid sequence SEQ ID NO: 203; a VL-CDR1 comprising the amino sequence SEQ ID NO: 205; a VL-CDR2 comprising the amino sequence SEQ ID NO: 206, and a VL-CDR3 comprising the amino sequence SEQ ID NO: 207; or
g. a VH-CDR1 comprising the amino acid sequence of SEQ ID NO: 201, a VH-CDR2 comprising the amino acid sequence of SEQ ID NO: 472 and a VH-CDR3 comprising the amino acid sequence SEQ ID NO: 203; a VL-CDR1 comprising the amino sequence SEQ ID NO: 435; a VL-CDR2 comprising the amino sequence SEQ ID NO: 206, and a VL-CDR3 comprising the amino sequence SEQ ID NO: 207.

In one embodiment the ASC binding molecule comprises:
a. a VH-CDR1 comprising the amino acid sequence of SEQ ID NO: 201, a VH-CDR2 comprising the amino acid sequence of SEQ ID NO: 202 and a VH-CDR3 comprising the amino acid sequence SEQ ID NO: 203; or
b. a VH-CDR1 comprising the amino acid sequence of SEQ ID NO: 201, a VH-CDR2 comprising the amino acid sequence of SEQ ID NO: 412 and a VH-CDR3 comprising the amino acid sequence SEQ ID NO: 203; or
c. a VH-CDR1 comprising the amino acid sequence of SEQ ID NO: 201, a VH-CDR2 comprising the amino acid sequence of SEQ ID NO: 462 and a VH-CDR3 comprising the amino acid sequence SEQ ID NO: 203; or
d. a VH-CDR1 comprising the amino acid sequence of SEQ ID NO: 201, a VH-CDR2 comprising the amino acid sequence of SEQ ID NO: 472 and a VH-CDR3 comprising the amino acid sequence SEQ ID NO: 203.

In one embodiment, the ASC binding molecule may comprise:
a. a VL-CDR1 comprising the amino acid sequence of SEQ ID NO: 205, a VL-CDR2 comprising the amino acid sequence of SEQ ID NO: 206, a VL-CDR3 comprising the amino acid sequence SEQ ID NO: 207; or
b. a VL-CDR1 comprising the amino acid sequence of SEQ ID NO: 435, a VL-CDR2 comprising the amino acid sequence of SEQ ID NO: 206, a VL-CDR3 comprising the amino acid sequence SEQ ID NO: 207.

In one embodiment the ASC binding molecule may comprise:
a. a Heavy Chain Variable Region (VH) comprising the amino acid sequence of SEQ ID NO: 400, or having at least 93%, 94%, 95%, 96%, 97%, 98% or 99% sequence identity to the amino acid sequence of SEQ ID NO: 400 and a Light Chain Variable Region (VL) comprising the amino acid sequence of SEQ ID NO: 404 or a Light Chain Variable Region (VL) having at least 95%, 96%, 97%, 98% or 99% sequence identity to the amino acid sequence of SEQ ID NO: 404; or
b. a Heavy Chain Variable Region (VH) comprising the amino acid sequence of SEQ ID NO: 410, or having at least 93%, 94%, 95%, 96%, 97%, 98% or 99% sequence identity to the amino acid sequence of SEQ ID NO: 410 and a Light Chain Variable Region (VL) comprising the amino acid sequence of SEQ ID NO: 414 or a Light Chain Variable Region (VL) having at least 95%, 96%, 97%, 98% or 99% sequence identity to the amino acid sequence of SEQ ID NO: 414; or
c. a Heavy Chain Variable Region (VH) comprising the amino acid sequence of SEQ ID NO: 420, or having at least 93%, 94%, 95%, 96%, 97%, 98% or 99% sequence identity to the amino acid sequence of SEQ ID NO: 420 and a Light Chain Variable Region (VL) comprising the amino acid sequence of SEQ ID NO: 424 or a Light Chain Variable Region (VL) having at least 95%, 96%, 97%, 98% or 99% sequence identity to the amino acid sequence of SEQ ID NO: 424; or
d. a Heavy Chain Variable Region (VH) comprising the amino acid sequence of SEQ ID NO: 430, or having at least 93%, 94%, 95%, 96%, 97%, 98% or 99% sequence identity to the amino acid sequence of SEQ ID NO: 430 and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
e. a Heavy Chain Variable Region (VH) comprising the amino acid sequence of SEQ ID NO: 440, or having at least 93%, 94%, 95%, 96%, 97%, 98% or 99% sequence identity to the amino acid sequence of SEQ ID NO: 440 and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
f. a Heavy Chain Variable Region (VH) comprising the amino acid sequence of SEQ ID NO: 450, or having at least 93%, 94%, 95%, 96%, 97%, 98% or 99% sequence identity to the amino acid sequence of SEQ ID NO: 450 and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
g. a Heavy Chain Variable Region (VH) comprising the amino acid sequence of SEQ ID NO: 460, or having at least 93%, 94%, 95%, 96%, 97%, 98% or 99% sequence identity to the amino acid sequence of SEQ ID NO: 460 and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
h. a Heavy Chain Variable Region (VH) comprising the amino acid sequence of SEQ ID NO: 470, or having at least 93%, 94%, 95%, 96%, 97%, 98% or 99% sequence identity to the amino acid sequence of SEQ ID NO: 470 and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
i. a Heavy Chain Variable Region (VH) comprising the amino acid sequence of SEQ ID NO: 480, or having at least 93%, 94%, 95%, 96%, 97%, 98% or 99% sequence identity to the amino acid sequence of SEQ ID NO: 480 and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
j. a Heavy Chain Variable Region (VH) comprising the amino acid sequence of SEQ ID NO: 490, or having at least 93%, 94%, 95%, 96%, 97%, 98% or 99% sequence identity to the amino acid sequence of SEQ ID NO: 490 and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
k. a Heavy Chain Variable Region (VH) comprising the amino acid sequence of SEQ ID NO: 500, or having at least 93%, 94%, 95%, 96%, 97%, 98% or 99% sequence identity to the amino acid sequence of SEQ ID NO: 500 and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
l. a Heavy Chain Variable Region (VH) comprising the amino acid sequence of SEQ ID NO: 510, or having at least 93%, 94%, 95%, 96%, 97%, 98% or 99% sequence identity to the amino acid sequence of SEQ ID NO: 510 and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
m. a Heavy Chain Variable Region (VH) comprising the amino acid sequence of SEQ ID NO: 520, or having at least 93%, 94%, 95%, 96%, 97%, 98% or 99% sequence identity to the amino acid sequence of SEQ ID NO: 520 and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
n. a Heavy Chain Variable Region (VH) comprising the amino acid sequence of SEQ ID NO: 530, or having at least 93%, 94%, 95%, 96%, 97%, 98% or 99% sequence identity to the amino acid sequence of SEQ ID NO: 530 and a Light Chain Variable Region (VL) comprising the amino acid sequence of SEQ ID NO: 424 or a Light Chain Variable Region (VL) having at least 95%, 96%, 97%, 98% or 99% sequence identity to the amino acid sequence of SEQ ID NO: 424; or
o. a Heavy Chain Variable Region (VH) comprising the amino acid sequence of SEQ ID NO: 540, or having at least 93%, 94%, 95%, 96%, 97%, 98% or 99% sequence identity to the amino acid sequence of SEQ ID NO: 540 and a Light Chain Variable Region (VL) comprising the amino acid sequence of SEQ ID NO: 424 or a Light Chain Variable Region (VL) having at least 95%, 96%, 97%, 98% or 99% sequence identity to the amino acid sequence of SEQ ID NO: 424; or
p. a Heavy Chain Variable Region (VH) comprising the amino acid sequence of SEQ ID NO: 400, or having at least 93%, 94%, 95%, 96%, 97%, 98% or 99% sequence identity to the amino acid sequence of SEQ ID NO: 400 and a Light Chain Variable Region (VL) comprising the amino acid sequence of SEQ ID NO: 414 or a Light Chain Variable Region (VL) having at least 95%, 96%, 97%, 98% or 99% sequence identity to the amino acid sequence of SEQ ID NO: 414; or
q. a Heavy Chain Variable Region (VH) comprising the amino acid sequence of SEQ ID NO: 410, or having at least 93%, 94%, 95%, 96%, 97%, 98% or 99% sequence identity to the amino acid sequence of SEQ ID NO: 410 and a Light Chain Variable Region (VL) comprising the amino acid sequence of SEQ ID NO: 404 or a Light Chain Variable Region (VL) having at least 95%, 96%, 97%, 98% or 99% sequence identity to the amino acid sequence of SEQ ID NO: 404; or
r. a Heavy Chain Variable Region (VH) comprising the amino acid sequence of SEQ ID NO: 410, or having at least 93%, 94%, 95%, 96%, 97%, 98% or 99% sequence identity to the amino acid sequence of SEQ ID NO: 410 and a Light Chain Variable Region (VL) comprising the amino acid sequence of SEQ ID NO: 424 or a Light Chain Variable Region (VL) having at least 95%, 96%, 97%, 98% or 99% sequence identity to the amino acid sequence of SEQ ID NO: 424; or
s. a Heavy Chain Variable Region (VH) comprising the amino acid sequence of SEQ ID NO: 410, or having at least 93%, 94%, 95%, 96%, 97%, 98% or 99% sequence identity to the amino acid sequence of SEQ ID NO: 410 and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
t. a Heavy Chain Variable Region (VH) comprising the amino acid sequence of SEQ ID NO: 420, or having at least 93%, 94%, 95%, 96%, 97%, 98% or 99% sequence identity to the amino acid sequence of SEQ ID NO: 420 and a Light Chain Variable Region (VL) comprising the amino acid sequence of SEQ ID NO: 404 or a Light Chain Variable Region (VL) having at least 95%, 96%, 97%, 98% or 99% sequence identity to the amino acid sequence of SEQ ID NO: 404; or
u. a Heavy Chain Variable Region (VH) comprising the amino acid sequence of SEQ ID NO: 420, or having at least 93%, 94%, 95%, 96%, 97%, 98% or 99% sequence identity to the amino acid sequence of SEQ ID NO: 420 and a Light Chain Variable Region (VL) comprising the amino acid sequence of SEQ ID NO: 414 or a Light Chain Variable Region (VL) having at least 95%, 96%, 97%, 98% or 99% sequence identity to the amino acid sequence of SEQ ID NO: 414; or
v. a Heavy Chain Variable Region (VH) comprising the amino acid sequence of SEQ ID NO: 430, or having at least 93%, 94%, 95%, 96%, 97%, 98% or 99% sequence identity to the amino acid sequence of SEQ ID NO: 430 and a Light Chain Variable Region (VL) comprising the amino acid sequence of SEQ ID NO: 404 or a Light Chain Variable Region (VL) having at least 95%, 96%, 97%, 98% or 99% sequence identity to the amino acid sequence of SEQ ID NO: 404; or
w. a Heavy Chain Variable Region (VH) comprising the amino acid sequence of SEQ ID NO: 430, or having at least 93%, 94%, 95%, 96%, 97%, 98% or 99% sequence identity to the amino acid sequence of SEQ ID NO: 430 and a Light Chain Variable Region (VL) comprising the amino acid sequence of SEQ ID NO: 414 or a Light Chain Variable Region (VL) having at least 95%, 96%, 97%, 98% or 99% sequence identity to the amino acid sequence of SEQ ID NO: 414; or
x. a Heavy Chain Variable Region (VH) comprising the amino acid sequence of SEQ ID NO: 400, or having at least 93%, 94%, 95%, 96%, 97%, 98% or 99% sequence identity to the amino acid sequence of SEQ ID NO: 400 and a Light Chain Variable Region (VL) comprising the amino acid sequence of SEQ ID NO: 424 or a Light Chain Variable Region (VL) having at least 95%, 96%, 97%, 98% or 99% sequence identity to the amino acid sequence of SEQ ID NO: 424; or
y. a Heavy Chain Variable Region (VH) comprising the amino acid sequence of SEQ ID NO: 450, or having at least 93%, 94%, 95%, 96%, 97%, 98% or 99% sequence identity to the amino acid sequence of SEQ ID NO: 450 and a Light Chain Variable Region (VL) comprising the amino acid sequence of SEQ ID NO: 424 or a Light Chain Variable Region (VL) having at least 95%, 96%, 97%, 98% or 99% sequence identity to the amino acid sequence of SEQ ID NO: 424; or
z. a Heavy Chain Variable Region (VH) comprising the amino acid sequence of SEQ ID NO: 480, or having at least 93%, 94%, 95%, 96%, 97%, 98% or 99% sequence identity to the amino acid sequence of SEQ ID NO: 480 and a Light Chain Variable Region (VL) comprising the amino acid sequence of SEQ ID NO: 424 or a Light Chain Variable Region (VL) having at least 95%, 96%, 97%, 98% or 99% sequence identity to the amino acid sequence of SEQ ID NO: 424; or
aa. a Heavy Chain Variable Region (VH) comprising the amino acid sequence of SEQ ID NO: 520, or having at least 93%, 94%, 95%, 96%, 97%, 98% or 99% sequence identity to the amino acid sequence of SEQ ID NO: 520 and a Light Chain Variable Region (VL) comprising the amino acid sequence of SEQ ID NO: 424 or a Light Chain Variable Region (VL) having at least 95%, 96%, 97%, 98% or 99% sequence identity to the amino acid sequence of SEQ ID NO: 424; or
bb. a Heavy Chain Variable Region (VH) comprising the amino acid sequence of SEQ ID NO: 430, or having at least 93%, 94%, 95%, 96%, 97%, 98% or 99% sequence identity to the amino acid sequence of SEQ ID NO: 430, and a Light Chain Variable Region (VL) comprising the amino acid sequence of SEQ ID NO: 424 or a Light Chain Variable Region (VL) having at least 95%, 96%, 97%, 98% or 99% sequence identity to the amino acid sequence of SEQ ID NO: 424.

In one embodiment the ASC binding molecule may comprise:
a. a Heavy Chain Variable Region (VH) comprising the amino acid sequence of SEQ ID NO: 400, or having at least 93%, 94%, 95%, 96%, 97%, 98% or 99% sequence identity to the amino acid sequence of SEQ ID NO: 400 and a Light Chain Variable Region (VL) comprising the amino acid sequence of SEQ ID NO: 404; or
b. a Heavy Chain Variable Region (VH) comprising the amino acid sequence of SEQ ID NO: 410, or having at least 93%, 94%, 95%, 96%, 97%, 98% or 99% sequence identity to the amino acid sequence of SEQ ID NO: 410 and a Light Chain Variable Region (VL) comprising the amino acid sequence of SEQ ID NO: 414; or
c. a Heavy Chain Variable Region (VH) comprising the amino acid sequence of SEQ ID NO: 420, or having at least 93%, 94%, 95%, 96%, 97%, 98% or 99% sequence identity to the amino acid sequence of SEQ ID NO: 420 and a Light Chain Variable Region (VL) comprising the amino acid sequence of SEQ ID NO: 424; or
d. a Heavy Chain Variable Region (VH) comprising the amino acid sequence of SEQ ID NO: 430, or having at least 93%, 94%, 95%, 96%, 97%, 98% or 99% sequence identity to the amino acid sequence of SEQ ID NO: 430 and a Light Chain Variable Region (VL) comprising the amino acid sequence of SEQ ID NO: 434; or
e. a Heavy Chain Variable Region (VH) comprising the amino acid sequence of SEQ ID NO: 440, or having at least 93%, 94%, 95%, 96%, 97%, 98% or 99% sequence identity to the amino acid sequence of SEQ ID NO: 440 and a Light Chain Variable Region (VL) comprising the amino acid sequence of SEQ ID NO: 434; or
f. a Heavy Chain Variable Region (VH) comprising the amino acid sequence of SEQ ID NO: 450, or having at least 93%, 94%, 95%, 96%, 97%, 98% or 99% sequence identity to the amino acid sequence of SEQ ID NO: 450 and a Light Chain Variable Region (VL) comprising the amino acid sequence of SEQ ID NO: 434; or
g. a Heavy Chain Variable Region (VH) comprising the amino acid sequence of SEQ ID NO: 460, or having at least 93%, 94%, 95%, 96%, 97%, 98% or 99% sequence identity to the amino acid sequence of SEQ ID NO: 460 and a Light Chain Variable Region (VL) comprising the amino acid sequence of SEQ ID NO: 434; or
h. a Heavy Chain Variable Region (VH) comprising the amino acid sequence of SEQ ID NO: 470, or having at least 93%, 94%, 95%, 96%, 97%, 98% or 99% sequence identity to the amino acid sequence of SEQ ID NO: 470 and a Light Chain Variable Region (VL) comprising the amino acid sequence of SEQ ID NO: 434; or
i. a Heavy Chain Variable Region (VH) comprising the amino acid sequence of SEQ ID NO: 480, or having at least 93%, 94%, 95%, 96%, 97%, 98% or 99% sequence identity to the amino acid sequence of SEQ ID NO: 480 and a Light Chain Variable Region (VL) comprising the amino acid sequence of SEQ ID NO: 434; or
j. a Heavy Chain Variable Region (VH) comprising the amino acid sequence of SEQ ID NO: 490, or having at least 93%, 94%, 95%, 96%, 97%, 98% or 99% sequence identity to the amino acid sequence of SEQ ID NO: 490 and a Light Chain Variable Region (VL) comprising the amino acid sequence of SEQ ID NO: 434; or
k. a Heavy Chain Variable Region (VH) comprising the amino acid sequence of SEQ ID NO: 500, or having at least 93%, 94%, 95%, 96%, 97%, 98% or 99% sequence identity to the amino acid sequence of SEQ ID NO: 500 and a Light Chain Variable Region (VL) comprising the amino acid sequence of SEQ ID NO: 434; or
l. a Heavy Chain Variable Region (VH) comprising the amino acid sequence of SEQ ID NO: 510, or having at least 93%, 94%, 95%, 96%, 97%, 98% or 99% sequence identity to the amino acid sequence of SEQ ID NO: 510 and a Light Chain Variable Region (VL) comprising the amino acid sequence of SEQ ID NO: 434; or
m. a Heavy Chain Variable Region (VH) comprising the amino acid sequence of SEQ ID NO: 520, or having at least 93%, 94%, 95%, 96%, 97%, 98% or 99% sequence identity to the amino acid sequence of SEQ ID NO: 520 and a Light Chain Variable Region (VL) comprising the amino acid sequence of SEQ ID NO: 434; or
n. a Heavy Chain Variable Region (VH) comprising the amino acid sequence of SEQ ID NO: 530, or having at least 93%, 94%, 95%, 96%, 97%, 98% or 99% sequence identity to the amino acid sequence of SEQ ID NO: 530 and a Light Chain Variable Region (VL) comprising the amino acid sequence of SEQ ID NO: 424; or
o. a Heavy Chain Variable Region (VH) comprising the amino acid sequence of SEQ ID NO: 540, or having at least 93%, 94%, 95%, 96%, 97%, 98% or 99% sequence identity to the amino acid sequence of SEQ ID NO: 540 and a Light Chain Variable Region (VL) comprising the amino acid sequence of SEQ ID NO: 424; or
p. a Heavy Chain Variable Region (VH) comprising the amino acid sequence of SEQ ID NO: 400, or having at least 93%, 94%, 95%, 96%, 97%, 98% or 99% sequence identity to the amino acid sequence of SEQ ID NO: 400 and a Light Chain Variable Region (VL) comprising the amino acid sequence of SEQ ID NO: 414; or
q. a Heavy Chain Variable Region (VH) comprising the amino acid sequence of SEQ ID NO: 410, or having at least 93%, 94%, 95%, 96%, 97%, 98% or 99% sequence identity to the amino acid sequence of SEQ ID NO: 410 and a Light Chain Variable Region (VL) comprising the amino acid sequence of SEQ ID NO: 404; or
r. a Heavy Chain Variable Region (VH) comprising the amino acid sequence of SEQ ID NO: 410, or having at least 93%, 94%, 95%, 96%, 97%, 98% or 99% sequence identity to the amino acid sequence of SEQ ID NO: 410 and a Light Chain Variable Region (VL) comprising the amino acid sequence of SEQ ID NO: 424; or
s. a Heavy Chain Variable Region (VH) comprising the amino acid sequence of SEQ ID NO: 410, or having at least 93%, 94%, 95%, 96%, 97%, 98% or 99% sequence identity to the amino acid sequence of SEQ ID NO: 410 and a Light Chain Variable Region (VL) comprising the amino acid sequence of SEQ ID NO: 434; or
t. a Heavy Chain Variable Region (VH) comprising the amino acid sequence of SEQ ID NO: 420, or having at least 93%, 94%, 95%, 96%, 97%, 98% or 99% sequence identity to the amino acid sequence of SEQ ID NO: 420 and a Light Chain Variable Region (VL) comprising the amino acid sequence of SEQ ID NO: 404; or
u. a Heavy Chain Variable Region (VH) comprising the amino acid sequence of SEQ ID NO: 420, or having at least 93%, 94%, 95%, 96%, 97%, 98% or 99% sequence identity to the amino acid sequence of SEQ ID NO: 420 and a Light Chain Variable Region (VL) comprising the amino acid sequence of SEQ ID NO: 414; or
v. a Heavy Chain Variable Region (VH) comprising the amino acid sequence of SEQ ID NO: 430, or having at least 93%, 94%, 95%, 96%, 97%, 98% or 99% sequence identity to the amino acid sequence of SEQ ID NO: 430 and a Light Chain Variable Region (VL) comprising the amino acid sequence of SEQ ID NO: 404; or
w. a Heavy Chain Variable Region (VH) comprising the amino acid sequence of SEQ ID NO: 430, or having at least 93%, 94%, 95%, 96%, 97%, 98% or 99% sequence identity to the amino acid sequence of SEQ ID NO: 430 and a Light Chain Variable Region (VL) comprising the amino acid sequence of SEQ ID NO: 414; or
x. a Heavy Chain Variable Region (VH) comprising the amino acid sequence of SEQ ID NO: 400, or having at least 93%, 94%, 95%, 96%, 97%, 98% or 99% sequence identity to the amino acid sequence of SEQ ID NO: 400 and a Light Chain Variable Region (VL) comprising the amino acid sequence of SEQ ID NO: 424; or
y. a Heavy Chain Variable Region (VH) comprising the amino acid sequence of SEQ ID NO: 450, or having at least 93%, 94%, 95%, 96%, 97%, 98% or 99% sequence identity to the amino acid sequence of SEQ ID NO: 450 and a Light Chain Variable Region (VL) comprising the amino acid sequence of SEQ ID NO: 424; or
z. a Heavy Chain Variable Region (VH) comprising the amino acid sequence of SEQ ID NO: 480, or having at least 93%, 94%, 95%, 96%, 97%, 98% or 99% sequence identity to the amino acid sequence of SEQ ID NO: 480 and a Light Chain Variable Region (VL) comprising the amino acid sequence of SEQ ID NO: 424; or
aa. a Heavy Chain Variable Region (VH) comprising the amino acid sequence of SEQ ID NO: 520, or having at least 93%, 94%, 95%, 96%, 97%, 98% or 99% sequence identity to the amino acid sequence of SEQ ID NO: 520 and a Light Chain Variable Region (VL) comprising the amino acid sequence of SEQ ID NO: 424; or
bb. a Heavy Chain Variable Region (VH) comprising the amino acid sequence of SEQ ID NO: 430, or having at least 93%, 94%, 95%, 96%, 97%, 98% or 99% sequence identity to the amino acid sequence of SEQ ID NO: 430, and a Light Chain Variable Region (VL) comprising the amino acid sequence of SEQ ID NO: 424.

In one embodiment the ASC binding molecule may comprise:
a. a Heavy Chain Variable Region (VH) comprising the amino acid sequence of SEQ ID NO: 400 and a Light Chain Variable Region (VL) comprising the amino acid sequence of SEQ ID NO: 404; or
b. a Heavy Chain Variable Region (VH) comprising the amino acid sequence of SEQ ID NO: 410 and a Light Chain Variable Region (VL) comprising the amino acid sequence of SEQ ID NO: 414; or
c. a Heavy Chain Variable Region (VH) comprising the amino acid sequence of SEQ ID NO: 420 and a Light Chain Variable Region (VL) comprising the amino acid sequence of SEQ ID NO: 424; or
d. a Heavy Chain Variable Region (VH) comprising the amino acid sequence of SEQ ID NO: 430 and a Light Chain Variable Region (VL) comprising the amino acid sequence of SEQ ID NO: 434; or
e. a Heavy Chain Variable Region (VH) comprising the amino acid sequence of SEQ ID NO: 440 and a Light Chain Variable Region (VL) comprising the amino acid sequence of SEQ ID NO: 434; or
f. a Heavy Chain Variable Region (VH) comprising the amino acid sequence of SEQ ID NO: 450 and a Light Chain Variable Region (VL) comprising the amino acid sequence of SEQ ID NO: 434; or
g. a Heavy Chain Variable Region (VH) comprising the amino acid sequence of SEQ ID NO: 460 and a Light Chain Variable Region (VL) comprising the amino acid sequence of SEQ ID NO: 434; or
h. a Heavy Chain Variable Region (VH) comprising the amino acid sequence of SEQ ID NO: 470 and a Light Chain Variable Region (VL) comprising the amino acid sequence of SEQ ID NO: 434; or
i. a Heavy Chain Variable Region (VH) comprising the amino acid sequence of SEQ ID NO: 480 and a Light Chain Variable Region (VL) comprising the amino acid sequence of SEQ ID NO: 434; or
j. a Heavy Chain Variable Region (VH) comprising the amino acid sequence of SEQ ID NO: 490 and a Light Chain Variable Region (VL) comprising the amino acid sequence of SEQ ID NO: 434; or
k. a Heavy Chain Variable Region (VH) comprising the amino acid sequence of SEQ ID NO: 500 and a Light Chain Variable Region (VL) comprising the amino acid sequence of SEQ ID NO: 434; or
l. a Heavy Chain Variable Region (VH) comprising the amino acid sequence of SEQ ID NO: 510 and a Light Chain Variable Region (VL) comprising the amino acid sequence of SEQ ID NO: 434; or
m. a Heavy Chain Variable Region (VH) comprising the amino acid sequence of SEQ ID NO: 520 and a Light Chain Variable Region (VL) comprising the amino acid sequence of SEQ ID NO: 434; or
n. a Heavy Chain Variable Region (VH) comprising the amino acid sequence of SEQ ID NO: 530 and a Light Chain Variable Region (VL) comprising the amino acid sequence of SEQ ID NO: 424; or
o. a Heavy Chain Variable Region (VH) comprising the amino acid sequence of SEQ ID NO: 540 and a Light Chain Variable Region (VL) comprising the amino acid sequence of SEQ ID NO: 424; or
p. a Heavy Chain Variable Region (VH) comprising the amino acid sequence of SEQ ID NO: 400 and a Light Chain Variable Region (VL) comprising the amino acid sequence of SEQ ID NO: 414; or
q. a Heavy Chain Variable Region (VH) comprising the amino acid sequence of SEQ ID NO: 410 and a Light Chain Variable Region (VL) comprising the amino acid sequence of SEQ ID NO: 404; or
r. a Heavy Chain Variable Region (VH) comprising the amino acid sequence of SEQ ID NO: 410 and a Light Chain Variable Region (VL) comprising the amino acid sequence of SEQ ID NO: 424; or
s. a Heavy Chain Variable Region (VH) comprising the amino acid sequence of SEQ ID NO: 410 and a Light Chain Variable Region (VL) comprising the amino acid sequence of SEQ ID NO: 434; or
t. a Heavy Chain Variable Region (VH) comprising the amino acid sequence of SEQ ID NO: 420 and a Light Chain Variable Region (VL) comprising the amino acid sequence of SEQ ID NO: 404; or
u. a Heavy Chain Variable Region (VH) comprising the amino acid sequence of SEQ ID NO: 420 and a Light Chain Variable Region (VL) comprising the amino acid sequence of SEQ ID NO: 414; or
v. a Heavy Chain Variable Region (VH) comprising the amino acid sequence of SEQ ID NO: 430 and a Light Chain Variable Region (VL) comprising the amino acid sequence of SEQ ID NO: 404; or
w. a Heavy Chain Variable Region (VH) comprising the amino acid sequence of SEQ ID NO: 430 and a Light Chain Variable Region (VL) comprising the amino acid sequence of SEQ ID NO: 414; or
x. a Heavy Chain Variable Region (VH) comprising the amino acid sequence of SEQ ID NO: 400 and a Light Chain Variable Region (VL) comprising the amino acid sequence of SEQ ID NO: 424; or
y. a Heavy Chain Variable Region (VH) comprising the amino acid sequence of SEQ ID NO: 450 and a Light Chain Variable Region (VL) comprising the amino acid sequence of SEQ ID NO: 424; or
z. a Heavy Chain Variable Region (VH) comprising the amino acid sequence of SEQ ID NO: 480 and a Light Chain Variable Region (VL) comprising the amino acid sequence of SEQ ID NO: 424; or
a. a Heavy Chain Variable Region (VH) comprising the amino acid sequence of SEQ ID NO: 520 and a Light Chain Variable Region (VL) comprising the amino acid sequence of SEQ ID NO: 424 or
aa. a Heavy Chain Variable Region (VH) comprising the amino acid sequence of SEQ ID NO: 430 and a Light Chain Variable Region (VL) comprising the amino acid sequence of SEQ ID NO: 424.

In one embodiment the ASC binding molecule may comprise:
a. a VH-CDR1 comprising the amino acid sequence of SEQ ID NO: 201, a VH-CDR2 comprising the amino acid sequence of SEQ ID NO: 202; and a VH-CDR3 comprising the amino acid sequence SEQ ID NO: 203, a VL-CDR1 comprising the amino sequence SEQ ID NO: 205; a VL-CDR2 comprising the amino sequence SEQ ID NO: 206, and a VL-CDR3 comprising the amino sequence SEQ ID NO: 207; or
b. a VH-CDR1 comprising the amino acid sequence of SEQ ID NO: 201, a VH-CDR2 comprising the amino acid sequence of SEQ ID NO: 412 and a VH-CDR3 comprising the amino acid sequence SEQ ID NO: 203; a VL-CDR1 comprising the amino sequence SEQ ID NO: 205; a VL-CDR2 comprising the amino sequence SEQ ID NO: 206, and a VL-CDR3 comprising the amino sequence SEQ ID NO: 207; or
c. a VH-CDR1 comprising the amino acid sequence of SEQ ID NO: 201, a VH-CDR2 comprising the amino acid sequence of SEQ ID NO: 462 and a VH-CDR3 comprising the amino acid sequence SEQ ID NO: 203; a VL-CDR1 comprising the amino sequence SEQ ID NO: 435; a VL-CDR2 comprising the amino sequence SEQ ID NO: 206, and a VL-CDR3 comprising the amino sequence SEQ ID NO: 207.

In one embodiment the ASC binding molecule may comprise:
a. a Heavy Chain Variable Region (VH) comprising the amino acid sequence of SEQ ID NO: 200, or having at least 93%, 94%, 95%, 96%, 97%, 98% or 99% sequence identity to the amino acid sequence of SEQ ID NO: 200 and a Light Chain Variable Region (VL) comprising the amino acid sequence of SEQ ID NO: 204 or a Light Chain Variable Region (VL) having at least 95%, 96%, 97%, 98% or 99% sequence identity to the amino acid sequence of SEQ ID NO: 204; or
b. a Heavy Chain Variable Region (VH) comprising the amino acid sequence of SEQ ID NO: 440, or having at least 93%, 94%, 95%, 96%, 97%, 98% or 99% sequence identity to the amino acid sequence of SEQ ID NO: 440 and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
c. a Heavy Chain Variable Region (VH) comprising the amino acid sequence of SEQ ID NO: 460, or having at least 93%, 94%, 95%, 96%, 97%, 98% or 99% sequence identity to the amino acid sequence of SEQ ID NO: 460 and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
d. a Heavy Chain Variable Region (VH) comprising the amino acid sequence of SEQ ID NO: 520, or having at least 93%, 94%, 95%, 96%, 97%, 98% or 99% sequence identity to the amino acid sequence of SEQ ID NO: 520 and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
e. a Heavy Chain Variable Region (VH) comprising the amino acid sequence of SEQ ID NO: 480, or having at least 93%, 94%, 95%, 96%, 97%, 98% or 99% sequence identity to the amino acid sequence of SEQ ID NO: 480 and a Light Chain Variable Region (VL) comprising the amino acid sequence of SEQ ID NO: 424 or a Light Chain Variable Region (VL) having at least 95%, 96%, 97%, 98% or 99% sequence identity to the amino acid sequence of SEQ ID NO: 424.

In one embodiment the ASC binding molecule may comprise:
a. a Heavy Chain Variable Region (VH) comprising the amino acid sequence of SEQ ID NO: 200, or having at least 93%, 94%, 95%, 96%, 97%, 98% or 99% sequence identity to the amino acid sequence of SEQ ID NO: 200 and a Light Chain Variable Region (VL) comprising the amino acid sequence of SEQ ID NO: 201; or
b. a Heavy Chain Variable Region (VH) comprising the amino acid sequence of SEQ ID NO: 440, or having at least 93%, 94%, 95%, 96%, 97%, 98% or 99% sequence identity to the amino acid sequence of SEQ ID NO: 440 and a Light Chain Variable Region (VL) comprising the amino acid sequence of SEQ ID NO: 434; or
c. a Heavy Chain Variable Region (VH) comprising the amino acid sequence of SEQ ID NO: 460, or having at least 93%, 94%, 95%, 96%, 97%, 98% or 99% sequence identity to the amino acid sequence of SEQ ID NO: 460 and a Light Chain Variable Region (VL) comprising the amino acid sequence of SEQ ID NO: 434; or
d. a Heavy Chain Variable Region (VH) comprising the amino acid sequence of SEQ ID NO: 520, or having at least 93%, 94%, 95%, 96%, 97%, 98% or 99% sequence identity to the amino acid sequence of SEQ ID NO: 520 and a Light Chain Variable Region (VL) comprising the amino acid sequence of SEQ ID NO: 434; or
e. a Heavy Chain Variable Region (VH) comprising the amino acid sequence of SEQ ID NO: 480, or having at least 93%, 94%, 95%, 96%, 97%, 98% or 99% sequence identity to the amino acid sequence of SEQ ID NO: 480 and a Light Chain Variable Region (VL) comprising the amino acid sequence of SEQ ID NO: 424.

In one embodiment the ASC binding molecule may comprise:
a. a Heavy Chain Variable Region (VH) comprising the amino acid sequence of SEQ ID NO: 200 and a Light Chain Variable Region (VL) comprising the amino acid sequence of SEQ ID NO: 204; or
b. a Heavy Chain Variable Region (VH) comprising the amino acid sequence of SEQ ID NO: 440 and a Light Chain Variable Region (VL) comprising the amino acid sequence of SEQ ID NO: 434; or
c. a Heavy Chain Variable Region (VH) comprising the amino acid sequence of SEQ ID NO: 460 and a Light Chain Variable Region (VL) comprising the amino acid sequence of SEQ ID NO: 434; or
d. a Heavy Chain Variable Region (VH) comprising the amino acid sequence of SEQ ID NO: 520 and a Light Chain Variable Region (VL) comprising the amino acid sequence of SEQ ID NO: 434; or
e. a Heavy Chain Variable Region (VH) comprising the amino acid sequence of SEQ ID NO: 480 and a Light Chain Variable Region (VL) comprising the amino acid sequence of SEQ ID NO: 424.

In one aspect, there is provided an immunoconjugate comprising the ASC binding molecule according to the invention.

In one aspect, the ASC binding molecule of the invention or immunoconjugate of the invention is for use in human or veterinary therapy.

In one embodiment, the ASC binding molecule or immunoconjugate for use of the invention is for the prevention, alleviation or treatment of a disease, disorder or condition associated with accumulation of ASC and/or ASC specks, preferably extracellular ASC specks.

In one embodiment, the ASC binding molecule or immunoconjugate of the invention, is for use in the prevention of a disease, disorder or condition associated with accumulation of ASC and/or ASC specks, preferably extracellular ASC specks.

In one embodiment, the ASC binding molecule or immunoconjugate of the invention is for use in postponing the onset of a disease, disorder or condition associated with accumulation of ASC and/or ASC specks, preferably extracellular ASC specks.

In one embodiment, the ASC binding molecule or immunoconjugate of the invention, is for use in the alleviation of a disease, disorder or condition associated with accumulation of ASC and/or ASC specks, preferably extracellular ASC specks.

In one embodiment, the ASC binding molecule or immunoconjugate of the invention, is for use in the treatment of a disease, disorder or condition associated with accumulation of ASC or ASC specks, preferably ASC specks, more preferably extracellular ASC specks.

In one embodiment, the ASC binding molecule or immunoconjugate of the invention is for use in the prevention, alleviation or treatment of a disease, disorder or condition associated with demyelination.

In one embodiment, the ASC binding molecule or immunoconjugate for use according to the invention is for use with a disease, disorder or condition associated with accumulation of accumulation of ASC or ASC specks, preferably ASC specks, more preferably extracellular ASC specks that is selected from either a central nervous system disease or peripheral inflammatory condition. The central nervous system disease is preferably Parkinson's disease, Alzheimer's disease, multiple sclerosis, amyotrophic lateral sclerosis, traumatic brain injury, spinal cord injury or chronic traumatic encephalopathy. The Peripheral inflammatory condition is preferably Non-Alcoholic SteatoHepatitis (NASH), Cryopyrin-associated periodic syndrome (CAPS), Chronic obstructive pulmonary disease (COPD), gout, psoriasis, acne, Hidradenitis Suppurativa (HS), Inflammatory Bowel Disease (IBD) (e.g. ulcerative colitis or Crohn's disease), Edema (DME), Geographic Atrophy (GA), Coronavirus-associated respiratory distress syndrome (CARDS), or Sjogren's Syndrome.

In one aspect, there is provided a method of human or veterinary therapy comprising administering an ASC binding molecule of the invention or immunoconjugate of the invention to a subject.

In one embodiment, the method of the invention comprises the prevention, alleviation or treatment of a disease, disorder or condition associated with accumulation of ASC and/or ASC specks, preferably extracellular ASC specks.

In one embodiment, the method of the invention comprises the prevention of a disease, disorder or condition associated with accumulation of ASC and/or ASC specks, preferably extracellular ASC specks.

In one embodiment, the method of the invention comprises the alleviation of a disease, disorder or condition associated with accumulation of ASC and/or ASC specks, preferably extracellular ASC specks.

In one embodiment, the method of the invention comprises the treatment of a disease, disorder or condition associated with accumulation of ASC or ASC specks, preferably ASC specks, more preferably extracellular ASC specks.

In one embodiment, the method of the invention comprises the postponement of the onset of a disease, disorder or condition associated with accumulation of ASC and/or ASC specks, preferably extracellular ASC specks.

In one embodiment, the method of the invention comprises the prevention, alleviation or treatment of a disease, disorder or condition associated with demyelination.

In one embodiment, the methods of the invention are for a disease, disorder or condition associated with accumulation of accumulation of ASC or ASC specks, preferably ASC specks, more preferably extracellular ASC specks, selected from a central nervous system disease or a peripheral inflammatory condition. The central nervous system disease is preferably Parkinson's disease, Alzheimer's disease, multiple sclerosis, amyotrophic lateral sclerosis, traumatic brain injury, spinal cord injury or chronic traumatic encephalopathy. The peripheral inflammatory condition is preferably Non-Alcoholic SteatoHepatitis (NASH), Cryopyrin-associated periodic syndrome (CAPS), Chronic obstructive pulmonary disease (COPD), gout, acne, Hidradenitis Suppurativa (HS), psoriasis, Inflammatory Bowel Disease (IBD) (e.g. ulcerative colitis or Crohn's disease), Edema (DME), Geographic Atrophy (GA), Coronavirus-associated respiratory distress syndrome (CARDS), or Sjogren's Syndrome.

In one embodiment, the method of the invention comprises the prevention or reduction of demyelination in a subject. The method may comprise administering the ASC binding molecule described herein or the immunoconjugate described herein to the subject. In one embodiment, the prevention or reduction of demyelination is improving demyelination score in vivo.

In one embodiment, the method of the invention comprises the reduction of levels of reactive microglia in a subject. The method may comprise administering the ASC binding molecule described herein or the immunoconjugate described herein to the subject.

In one embodiment, the method of the invention comprises the reduction of levels of ASC and/or cleaved capase-1 protein in a subject. The method may comprise administering the ASC binding molecule described herein or the immunoconjugate described herein to the subject.

In one embodiment, the method of the invention comprises the reduction of levels of infiltrating CD4+ T-cells in the spinal cord of a subject. The method may comprise administering the ASC binding molecule described herein or the immunoconjugate described herein to the subject.

In one aspect, there is provided an ASC binding molecule of the invention or immunoconjugate of the invention for use in diagnosis. The diagnosis may be in vivo diagnosis or in vitro diagnosis.

In one aspect, there is provided an ASC binding molecule of the invention or immunoconjugate of the invention for use in diagnosis of a disease, disorder or condition associated with ASC-dependent inflammation. The diagnosis may be in vivo diagnosis or in vitro diagnosis.

In one aspect, there is provided a method of detecting non-polymerized ASC and/or ASC specks in a sample obtained from a subject, the method comprising contacting the sample with the ASC binding molecule of the invention and detecting binding of the ASC binding molecule to non-polymerized ASC and/or ASC specks in the sample.

In one aspect, there is provided a method of quantifying non-polymerized ASC and/or ASC specks in a sample obtained from a subject, the method comprising contacting the sample with the ASC binding molecule of the invention or immunoconjugate of the invention and quantifying non-polymerized ASC and/or ASC specks in a sample based on the level of binding of the ASC binding molecule to non-polymerized ASC and/or ASC specks.

In one aspect, there is provided a method for diagnosing a disease, disorder or condition associated with ASC-dependent inflammation comprising performing the method of quantifying non-polymerized ASC and/or ASC specks in a sample obtained from a subject according to the invention, wherein higher levels of non-polymerized ASC and/or ASC specks in the sample compared with a control level based on healthy subjects are indicative of a disease, disorder or condition associated with ASC-dependent inflammation.

In one aspect, there is provided a diagnostic composition comprising the ASC binding molecule of the invention or immunoconjugate of the invention and an acceptable carrier and/or excipient. The diagnostic compositions of the invention may be used in all relevant methods according to the invention.

In one aspect, there is provided a pharmaceutical composition comprising the ASC binding molecule of the invention or immunoconjugate of the invention, and a pharmaceutically acceptable carrier and/or excipient.

In one aspect, there is provided a nucleic acid encoding the ASC binding molecule of the invention or immunoconjugate of the invention. The pharmaceutical compositions of the invention may be used in all relevant methods according to the invention.

In one aspect, there is provided a nucleic acid comprising a nucleotide sequence as provided in SEQ ID NO: 18, SEQ ID NO: 19, SEQ ID NO: 28, SEQ ID NO: 29, SEQ ID NO: 38, SEQ ID NO: 39, SEQ ID NO: 48, SEQ ID NO: 49, SEQ ID NO: 58, SEQ ID NO: 59, SEQ ID NO: 68, SEQ ID NO: 69, SEQ ID NO: 78, SEQ ID NO: 79, SEQ ID NO: 88, SEQ ID NO: 89, SEQ ID NO: 98, SEQ ID NO: 99, SEQ ID NO: 118, SEQ ID NO: 119, SEQ ID NO: 128, SEQ ID NO: 129, SEQ ID NO: 138, SEQ ID NO: 139, SEQ ID NO: 148, SEQ ID NO: 149, SEQ ID NO: 158, SEQ ID NO: 159, SEQ ID NO: 168, SEQ ID NO: 169, SEQ ID NO: 178, SEQ ID NO: 179, SEQ ID NO: 188, SEQ ID NO: 189, SEQ ID NO: 198, SEQ ID NO: 199, SEQ ID NO: 208, SEQ ID NO: 209, SEQ ID NO: 218, SEQ ID NO: 219, SEQ ID NO: 228, SEQ ID NO: 229, SEQ ID NO: 238, SEQ ID NO: 239, SEQ ID NO: 248, SEQ ID NO: 249, SEQ ID NO: 258, SEQ ID NO: 259, SEQ ID NO: 268, SEQ ID NO: 269, SEQ ID NO: 278, SEQ ID NO: 279, SEQ ID NO: 288, SEQ ID NO: 289, SEQ ID NO: 298, SEQ ID NO: 299, SEQ ID NO: 308, SEQ ID NO: 309, SEQ ID NO: 318, SEQ ID NO: 319, SEQ ID NO: 328, SEQ ID NO: 329, SEQ ID NO: 338, SEQ ID NO: 339, SEQ ID NO: 348, SEQ ID NO: 349, SEQ ID NO: 358, SEQ ID NO: 359, SEQ ID NO: 368, SEQ ID NO: 369, SEQ ID NO: 378, SEQ ID NO: 379, SEQ ID NO: 388, SEQ ID NO: 389, SEQ ID NO: 398 and SEQ ID NO: 399.

Additionally or alternatively, a nucleic acid comprising a nucleotide sequence of SEQ ID NO: 408, SEQ ID NO: 418, SEQ ID NO: 428, SEQ ID NO: 438, SEQ ID NO: 448, SEQ ID NO: 458, SEQ ID NO: 468, SEQ ID NO: 478, SEQ ID NO: 488, SEQ ID NO: 498, SEQ ID NO: 508, SEQ ID NO: 518, SEQ ID NO: 528, SEQ ID NO: 538, SEQ ID NO: 548, SEQ ID NO: 409, SEQ ID NO: 419, SEQ ID NO: 429 and SEQ ID NO: 439, SEQ ID NO: 558 and SEQ ID NO: 559 is provided.

In one embodiment, there is provided a nucleic acid comprising a nucleotide sequence as provided in SEQ ID NO:200, SEQ ID NO:204, SEQ ID NO:429, SEQ ID NO: 439, SEQ ID NO:448, SEQ ID NO: 468, SEQ ID NO:488 and SEQ ID NO:528.

In one aspect, there is provided a recombinant vector comprising the nucleic acid of the invention.

In one aspect, there is provided a host cell comprising the nucleic acid of the invention and/or the recombinant vector of the invention.

In one aspect, there is provided an isolated host cell that expresses the ASC binding molecule of the invention or immunoconjugate of the invention.

In one aspect, there is provided a method for producing an ASC binding molecule, comprising the steps of culturing the host cell of the invention under conditions suitable for producing the ASC binding molecule, and recovering the ASC binding molecule.

In one aspect, there is provided a kit for diagnosis of a disease, disorder or condition associated with ASC-dependent inflammation, or a kit for use in a method of the invention, comprising the ASC binding molecule of any one of the invention or immunoconjugate of the invention and a container.

### DETAILED DESCRIPTION

The present invention provides ASC binding molecules with various useful properties.

In one embodiment, the ASC binding molecule binds preferentially to ASC specks over non-polymerized ASC. In another embodiment, the ASC binding molecule binds preferentially to non-polymerized ASC over ASC specks. In another embodiment, the ASC binding molecule binds preferentially to ASC specks and does not bind to non-polymerized ASC. In another embodiment, the ASC binding molecule binds preferentially to non-polymerized ASC and does not bind to ASC specks. A suitable assay for the assessment of preferential binding is provided in Example 6, with results given in Table 8 (immunofluorescence for human ASC) and Table 9 (immunofluorescence for mouse ASC).

In some embodiments, the binding molecule binds non-polymerized ASC and does not bind to ASC specks. In some embodiments, the ASC binding molecule prevents or inhibits ASC polymerization. The ASC binding molecule may inhibit human ASC polymerization and/or mouse ASC polymerization. ASC polymerization may be measured in vitro, preferably by an ASC polymerization assay. In one embodiment, an ASC binding molecule inhibits human ASC polymerization with an IC50 below 33 nM, preferably 20 nM, more preferably 6.3 nM, even more preferably below 3.1 nM. In one embodiment, an ASC binding molecule inhibits mouse ASC polymerization with an IC50 below 61 nM, preferably 35.7 nM, more preferably below 22 nM. The ASC polymerization IC50 may be measured in accordance with a recombinant ASC polymerization assay, such as Example 7.

In some embodiments, the ASC binding molecule may have a functional efficacy in inhibition of human ASC (IC50 around 5 nM) and/or mouse ASC (IC50 around 30 nM) recombinant ASC polymerization. A suitable assay to assess ASC polymerization is disclosed in Example 7.

In some embodiments, the ASC binding molecule prevents or inhibits ASC dependent propagation of inflammation. The ASC dependent propagation of inflammation may be measured in vitro or in vivo. In one embodiment, the prevention or inhibition of ASC dependent propagation of inflammation is prevention or inhibition of IL-1β release. In one embodiment, the anti-ASC antibody or an antigen-binding fragment thereof that inhibits IL-1β release with a IC50 below 60 nM, preferably, 42 nM, more preferably 33 nM, even more preferably 17 nM in accordance with a suitable assay, as disclosed in Example 9. In one embodiment, the anti-ASC antibody inhibits IL-1β release by at least 20%, at least 30%, at least 40%, at least 50%, at least 60%, at least 70% at least 80%, or at least 90% as compared to a control. The control may be an isotype control antibody in some embodiments.

In some embodiments, the ASC binding molecule increases uptake of ASC extracellular specks by phagocytic cells such as macrophages or microglias. Uptake may be assessed in phagocytic cells such as macrophages or microglia differentiated from human monocytic cell lines. Examples 8 and 9 provide suitable means that demonstrating the assessment of macrophage uptake.

In some embodiments, the ASC binding molecule prevents or inhibits accumulation of ASC and/or ASC specks. The ASC speck accumulation may be intracellular or extracellular. Accumulation may be measured by conventional means such as western blotting or immunofluorescence. Accumulation may also be measured by a combination of means selected from the Examples disclosed herein.

In one embodiment, the ASC binding molecule prevents, reduces or inhibits demyelination.

The term “demyelination” is intended to encompass damage to the protective covering (myelin sheath) that surrounds nerve fibers in your brain, the nerves leading to the eyes (optic nerves) and spinal cord. When the myelin sheath is damaged, nerve impulses slow or even stop, causing neurological problems.

In one embodiment, the ASC binding molecule reduces demyelination by at least 10%, at least 15%, at least 20%, at least 25% at least 30%, at least 35%, at least 40%, at least 45%, at least 50%, at least 55%, at least 60%, at least 65%, at least 70%, at least 75%, at least 80%, or at least 85% as compared to a control (with no administration of the ASC binding molecule).

In one embodiment, the ASC binding molecule prevents demyelination above 10% of a tested area (e.g. from a sample collected from a cervical, thoracic and/or lumbar segment of the spinal cord). In one embodiment, the ASC binding molecule prevents demyelination above 15% of a tested area. That is to say that the ASC binding molecule keeps demyelination of the nerve fibers below 10%, or below 15% for a period of time. Preferably, the ASC binding molecule keeps demyelination of the nerve fibers below 5% for a period of time. The period of time may by at least 1 week, at least 1 month, at least 1 year, at least 2 years, at least 5 years, at least 10 years, at least 15 years, at least 20 years, or for as long as the ASC binding molecule is administered to the subject. Thus, in one embodiment, the ASC binding molecule may postpone demyelination of the nerve fibers by at least 1 week, at least 1 month, at least 1 year, at least 2 years, at least 5 years, at least 10 years, at least 15 years, at least 20 years, or for as long as the ASC binding molecule is administered to the subject. In one embodiment, the ASC binding molecule may postpone the onset of a disease, disorder or condition associated with demyelination of the nerve fibers by at least 1 week, at least 1 month, at least 1 year, at least 2 years, at least 5 years, at least 10 years, at least years, at least 20 years, or for as long as the ASC binding molecule is administered to the subject. In one embodiment, the ASC binding molecule preventing, reducing or inhibiting demyelination is ACI-8016-32B6C7-AB1.

In one embodiment, prevention, reduction, or inhibition of demyelination is improving demyelination score in vivo. The score may rely on a scale of 0-5 as follows:
0—no demyelination (less than 2% demyelinated area)
1-2 to 5% demyelinated area
2-6 to 19% demyelinated area
3-20 to 29% demyelinated area
4-30 to 50% demyelinated area
5>50% demyelinated area

Thus, the term “improving demyelination score” may mean reducing the score. For example, the score may be reduced from 3 to 1 so that the demyelinated area is reduced to 2-5%.

The score may rely on the improvement of clinical observations as follows:
0—No obvious changes in motor functions of the mouse in comparison to non-immunized mice
1—Limp tail;
2—Limp tail and weakness of hind legs;
3—Limp tail and complete paralysis of hind legs (most common); or limp tail with paralysis of one front and one hind leg; or all of them;
4—Limp tail, complete hind leg, and partial front leg paralysis;
5—Complete hind and complete front leg paralysis, no movement around the cage; or mouse is spontaneously rolling in the cage; or the mouse is found dead due to paralysis.

The ASC binding molecule may increase the spleen mass in vivo. For example, the spleen mass may be increased by at least 10%, at least 15%, at least 20%, at least 25%, at least 30%, at least 35%, at least 40%, at least 45%, at least 50%, at least 55%, at least 60%, at least 65%, at least 70%, or at least 75% as compared to a control (with no ASC binding molecule administration, e.g. using IgG2a isotype control, as per FIG. 8C). In one embodiment, the ASC binding molecule reduces levels of infiltrating CD4+ T-cells escaping the spleen. In one embodiment, the ASC binding molecule reduces levels of infiltrating CD4+ T-cells in the spinal cord in vivo. For example, the ASC binding molecule may reduce levels of infiltrating CD4+ T-cells in the spinal cord by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 95%, or at least 100% as compared to a control (with no ASC binding molecule administration, e.g. using IgG2a isotype control, as per FIG. 9B). In one embodiment, the ASC binding molecule reducing levels of infiltrating CD4+ T-cells in the spinal cord is ACI-8016-32B6C7-AB1 or ACI-8016-18F4C12-AB1.

In one embodiment, the ASC binding molecule reduces levels of reactive microglia in vivo. For example, the ASC binding molecule may reduce levels of reactive microglia by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 95%, or at least 100% as compared to a control (with no ASC binding molecule administration, e.g. using IgG2a isotype control, as per FIG. 9C). In one embodiment, the ASC binding molecule reducing levels of reactive microglia in vivo is ACI-8016-32B6C7-AB1.

In one embodiment, the ASC binding molecule reduces levels of ASC and/or cleaved capase-1 protein in vivo. For example, the ASC binding molecule may reduce levels of ASC and/or cleaved capase-1 protein by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 95%, or at least 100% as compared to a control (with no ASC binding molecule administration, e.g. using IgG2a isotype control, as per FIG. 10). In one embodiment, the ASC binding molecule reducing levels of ASC and/or cleaved capase-1 protein in vivo is ACI-8016-32B6C7-AB1.

In one embodiment, the ASC binding molecule binds to an epitope in the ASC PYD domain comprising, consisting essentially of, or consisting of amino acid residues:
a) L9, D10, E13, N14, E18 and E19,
b) L9, D10, E13 and N14,
c) E13 and N14,
d) Q79, E80, G83 and Q84; or
e) E18, E19, V30, P31, N71, R74, D75, G77, Q79 and E80.

The amino acids are stated with reference to the amino acid sequence of human ASC of SEQ ID NO: 1. The ASC binding molecule may bind to an epitope in the ASC PYD domain comprising, consisting essentially of, or consisting of amino acid residues numbered:
a) 9, 10, 13, 14, 18 and 19,
b) 9, 10, 13 and 14,
c) 13 and 14,
d) 79, 80, 83 and 84, or
e) 18, 19, 30, 31, 71, 74, 75, 77, 79 and 80
   of human ASC of SEQ ID NO: 1.

As described in example 11, the epitopes may be defined using alanine scanning mutagenesis. Mutants of ASC, in particular the PYD domain of PYCARD, may be employed. Binding of the ASC binding molecules to mutants may be measured by a suitable immunoassay, such as an ELISA. The residues listed are those critical to binding, which may be defined as any appropriate loss of binding, such as retaining no more than 30% binding compared to a wild type control, in the presence of an alanine mutation at that position.

Alternatively, the ASC binding molecules may bind to an epitope in the ASC CARD domain comprising, consisting essentially of, or consisting of amino acid residues:
a) K174 and D175,
b) 1115, D116, R119, A120, K174, D175, S184, Q185, S186 and Y187,
c) 1115, D116, N170, W171, T172, K174, D175, S186 and Y187,
d) Y137,
e) R119, A120, L178, Q179, S186 and Y187 or
f) R119, A120, K174, D175, S186 and Y187.

The amino acids are stated with reference to the amino acid sequence of human ASC of SEQ ID NO: 1. The ASC binding molecule may bind to an epitope in the ASC CARD domain comprising, consisting essentially of, or consisting of amino acid residues numbered:
a) 174, and 175,
b) 115, 116, 119, 120, 174, 175, 184, 186 and 187,
c) 115, 116, 170, 171, 172, 174, 175, 186 and 187,
d) 137,
e) 119, 120, 178, 179, 186 and 187 or
f) 119, 120, 174, 175, 186 and 187
   of human ASC of SEQ ID NO: 1.

As described in example 13, the epitopes may be defined using alanine mutagenesis. Mutants of ASC, in particular the CARD domain of PYCARD, may be employed. Binding of the ASC binding molecules to mutants may be measured by a suitable immunoassay, such as an ELISA. The residues listed are those critical to binding, which may be defined as any appropriate loss of binding, such as retaining no more than 30% binding compared to a wild type control, in the presence of an alanine mutation at that position.

In one embodiment the ASC binding molecule may bind to an epitope comprising, consisting generally of or consisting of, amino acid residue numbered 174 with reference to the amino acid sequence of human ASC of SEQ ID NO: 1.

In one embodiment the ASC binding molecule may bind to an epitope comprising, consisting generally of or consisting of, amino acid residue numbered 175 with reference to the amino acid sequence of human ASC of SEQ ID NO: 1.

In one embodiment the ASC binding molecule may bind to an epitope comprising, consisting generally of or consisting of, amino acid residue numbered 115 with reference to the amino acid sequence of human ASC of SEQ ID NO: 1.

In one embodiment the ASC binding molecule may bind to an epitope comprising, consisting generally of or consisting of, amino acid residue numbered 116 with reference to the amino acid sequence of human ASC of SEQ ID NO: 1.

In one embodiment the ASC binding molecule may bind to an epitope comprising, consisting generally of or consisting of, amino acid residue numbered 119 with reference to the amino acid sequence of human ASC of SEQ ID NO: 1.

In one embodiment the ASC binding molecule may bind to an epitope comprising, consisting generally of or consisting of, amino acid residue numbered 120 with reference to the amino acid sequence of human ASC of SEQ ID NO: 1.

In one embodiment the ASC binding molecule may bind to an epitope comprising, consisting generally of or consisting of, amino acid residue numbered 170 with reference to the amino acid sequence of human ASC of SEQ ID NO: 1 In one embodiment the ASC binding molecule may bind to an epitope comprising, consisting generally of or consisting of, amino acid residue numbered 184 with reference to the amino acid sequence of human ASC of SEQ ID NO: 1.

In one embodiment the ASC binding molecule may bind to an epitope comprising, consisting generally of or consisting of, amino acid residue numbered 186 with reference to the amino acid sequence of human ASC of SEQ ID NO: 1.

In one embodiment the ASC binding molecule may bind to an epitope comprising, consisting generally of or consisting of, amino acid residue numbered 187 with reference to the amino acid sequence of human ASC of SEQ ID NO: 1.

In one embodiment the ASC binding molecule may bind to an epitope comprising, consisting generally of or consisting of, amino acid residue numbered 171 with reference to the amino acid sequence of human ASC of SEQ ID NO: 1.

In one embodiment the ASC binding molecule may bind to an epitope comprising, consisting generally of or consisting of, amino acid residue numbered 172 with reference to the amino acid sequence of human ASC of SEQ ID NO: 1.

In one embodiment the ASC binding molecule may bind to an epitope comprising, consisting generally of or consisting of, amino acid residue numbered 137 with reference to the amino acid sequence of human ASC of SEQ ID NO: 1.

In one embodiment the ASC binding molecule may bind to an epitope comprising, consisting generally of or consisting of, amino acid residue numbered 178 with reference to the amino acid sequence of human ASC of SEQ ID NO: 1.

In one embodiment the ASC binding molecule may bind to an epitope comprising, consisting generally of or consisting of, amino acid residue numbered 179 with reference to the amino acid sequence of human ASC of SEQ ID NO: 1 In one preferred embodiment the ASC binding molecule may bind to an epitope comprising, consisting generally of or consisting of, amino acid residues numbered 174 and 175 with reference to the amino acid sequence of human ASC of SEQ ID NO: 1.

In one embodiment the ASC binding molecule may bind to an epitope comprising, amino acid residues numbered 115 and 116 with reference to the amino acid sequence of human ASC of SEQ ID NO: 1.

In one embodiment the ASC binding molecule may bind to an epitope comprising amino acid residues numbered 119 and 120 with reference to the amino acid sequence of human ASC of SEQ ID NO: 1.

In one embodiment the ASC binding molecule may bind to an epitope comprising amino acid residues numbered 170, 171 and 172 with reference to the amino acid sequence of human ASC of SEQ ID NO: 1.

In one embodiment the ASC binding molecule may bind to an epitope comprising amino acid residues numbered 186 and 187 with reference to the amino acid sequence of human ASC of SEQ ID NO: 1.

In one embodiment the ASC binding molecule may bind to an epitope comprising, consisting generally of or consisting of, amino acid residues numbered 115, 116, 119, 120, 174, 175, 184, 186 and 187 with reference to the amino acid sequence of human ASC of SEQ ID NO: 1.

In one embodiment the ASC binding molecule may bind to an epitope comprising, consisting generally of or consisting of, amino acid residues numbered 115, 116, 170, 171, 172, 174, 175, 186 and 187 with reference to the amino acid sequence of human ASC of SEQ ID NO: 1.

In another embodiment the ASC binding molecule may bind to an epitope comprising, consisting generally of or consisting of, amino acid residue numbered 137 with reference to the amino acid sequence of human ASC of SEQ ID NO: 1.

In one embodiment the ASC binding molecule may bind to an epitope comprising, consisting generally of or consisting of, amino acid residues numbered 119, 120, 178, 179, 186 and 187 with reference to the amino acid sequence of human ASC of SEQ ID NO: 1.

In one embodiment the ASC binding molecule may bind to an epitope comprising, consisting generally of or consisting of, amino acid residues numbered 119, 120, 174, 175, 186 and 187 with reference to the amino acid sequence of human ASC of SEQ ID NO: 1.

In one embodiment, an ASC binding molecule, particularly an anti-ASC antibody or an antigen-binding fragment thereof of the invention comprises:
a. a VH-CDR1 comprising the amino acid sequence of SEQ ID NO: 11, a VH-CDR2 comprising the amino acid sequence of SEQ ID NO: 12, a VH-CDR3 comprising the amino acid sequence NEV (Asn-Glu-Val), a VL-CDR1 comprising the amino sequence SEQ ID NO: 15, a VL-CDR2 comprising the amino acid sequence of SEQ ID NO: 16, and a VL-CDR3 comprising the amino acid sequence of SEQ ID NO: 17; or
b. a VH-CDR1 comprising the amino acid sequence of SEQ ID NO: 21, a VH-CDR2 comprising the amino acid sequence of SEQ ID NO: 22, a VH-CDR3 comprising the amino acid sequence SEQ ID NO: 23, a VL-CDR1 comprising the amino sequence SEQ ID NO: 25, a VL-CDR2 comprising the amino acid sequence of SEQ ID NO: 26, and a VL-CDR3 comprising the amino acid sequence of SEQ ID NO: 27; or
c. a VH-CDR1 comprising the amino acid sequence of SEQ ID NO: 31, a VH-CDR2 comprising the amino acid sequence of SEQ ID NO: 32, a VH-CDR3 comprising the amino acid sequence of SEQ ID NO: 33, a VL-CDR1 comprising the amino sequence SEQ ID NO: 35, a VL-CDR2 comprising the amino acid sequence of SEQ ID NO: 36, and a VL-CDR3 comprising the amino acid sequence of SEQ ID NO: 37; or
d. a VH-CDR1 comprising the amino acid sequence of SEQ ID NO: 41, a VH-CDR2 comprising the amino acid sequence of SEQ ID NO: 42, a VH-CDR3 comprising the amino acid sequence SEQ ID NO: 43, a VL-CDR1 comprising the amino sequence SEQ ID NO: 45, a VL-CDR2 comprising the amino acid sequence of SEQ ID NO: 46, and a VL-CDR3 comprising the amino acid sequence of SEQ ID NO: 47; or
e. a VH-CDR1 comprising the amino acid sequence of SEQ ID NO: 51, a VH-CDR2 comprising the amino acid sequence of SEQ ID NO: 52, a VH-CDR3 comprising the amino acid sequence SEQ ID NO: 53, a VL-CDR1 comprising the amino sequence SEQ ID NO: 55, a VL-CDR2 comprising the amino acid sequence of SEQ ID NO: 56, and a VL-CDR3 comprising the amino acid sequence of SEQ ID NO: 57; or
f. a VH-CDR1 comprising the amino acid sequence of SEQ ID NO: 61, a VH-CDR2 comprising the amino acid sequence of SEQ ID NO: 62, a VH-CDR3 comprising the amino acid sequence SEQ ID NO: 63, a VL-CDR1 comprising the amino sequence SEQ ID NO: 65, a VL-CDR2 comprising the amino acid sequence of SEQ ID NO: 66, and a VL-CDR3 comprising the amino acid sequence of SEQ ID NO: 67; or
g. a VH-CDR1 comprising the amino acid sequence of SEQ ID NO: 71, a VH-CDR2 comprising the amino acid sequence of SEQ ID NO: 72, a VH-CDR3 comprising the amino acid sequence SEQ ID NO: 73, a VL-CDR1 comprising the amino sequence SEQ ID NO: 75, a VL-CDR2 comprising the amino acid sequence of SEQ ID NO: 76, and a VL-CDR3 comprising the amino acid sequence of SEQ ID NO: 77; or
h. a VH-CDR1 comprising the amino acid sequence of SEQ ID NO: 81, a VH-CDR2 comprising the amino acid sequence of SEQ ID NO: 82, a VH-CDR3 comprising the amino acid sequence SEQ ID NO: 83, a VL-CDR1 comprising the amino sequence SEQ ID NO: 85, a VL-CDR2 comprising the amino acid sequence of SEQ ID NO: 86, and a VL-CDR3 comprising the amino acid sequence of SEQ ID NO: 87; or
i. a VH-CDR1 comprising the amino acid sequence of SEQ ID NO: 91, a VH-CDR2 comprising the amino acid sequence of SEQ ID NO: 92, a VH-CDR3 comprising the amino acid sequence SEQ ID NO: 93, a VL-CDR1 comprising the amino sequence SEQ ID NO: 95, a VL-CDR2 comprising the amino acid sequence of SEQ ID NO: 96, and a VL-CDR3 comprising the amino acid sequence of SEQ ID NO: 97; or
j. a VH-CDR1 comprising the amino acid sequence of SEQ ID NO: 111, a VH-CDR2 comprising the amino acid sequence of SEQ ID NO: 112, a VH-CDR3 comprising the amino acid sequence SEQ ID NO: 113, a VL-CDR1 comprising the amino sequence SEQ ID NO: 115, a VL-CDR2 comprising the amino acid sequence of SEQ ID NO: 116, and a VL-CDR3 comprising the amino acid sequence of SEQ ID NO: 117; or
k. a VH-CDR1 comprising the amino acid sequence of SEQ ID NO: 121, a VH-CDR2 comprising the amino acid sequence of SEQ ID NO: 122, a VH-CDR3 comprising the amino acid sequence SEQ ID NO: 123, a VL-CDR1 comprising the amino sequence SEQ ID NO: 125, a VL-CDR2 comprising the amino acid sequence of SEQ ID NO: 126, and a VL-CDR3 comprising the amino acid sequence of SEQ ID NO: 127; or
l. a VH-CDR1 comprising the amino acid sequence of SEQ ID NO: 131, a VH-CDR2 comprising the amino acid sequence of SEQ ID NO: 132, a VH-CDR3 comprising the amino acid sequence SEQ ID NO: 133, a VL-CDR1 comprising the amino sequence SEQ ID NO: 135, a VL-CDR2 comprising the amino acid sequence of SEQ ID NO: 66, and a VL-CDR3 comprising the amino acid sequence of SEQ ID NO: 137; or
m. a VH-CDR1 comprising the amino acid sequence of SEQ ID NO: 111, a VH-CDR2 comprising the amino acid sequence of SEQ ID NO: 142, a VH-CDR3 comprising the amino acid sequence SEQ ID NO: 143, a VL-CDR1 comprising the amino sequence SEQ ID NO: 145, a VL-CDR2 comprising the amino acid sequence of SEQ ID NO: 66, and a VL-CDR3 comprising the amino acid sequence of SEQ ID NO: 147; or
n. a VH-CDR1 comprising the amino acid sequence of SEQ ID NO: 151, a VH-CDR2 comprising the amino acid sequence of SEQ ID NO: 152, a VH-CDR3 comprising the amino acid sequence SEQ ID NO: 153, a VL-CDR1 comprising the amino sequence SEQ ID NO: 155, a VL-CDR2 comprising the amino acid sequence of SEQ ID NO: 156, and a VL-CDR3 comprising the amino acid sequence of SEQ ID NO: 157; or
o. a VH-CDR1 comprising the amino acid sequence of SEQ ID NO: 161, a VH-CDR2 comprising the amino acid sequence of SEQ ID NO: 162, a VH-CDR3 comprising the amino acid sequence SEQ ID NO: 163, a VL-CDR1 comprising the amino sequence SEQ ID NO: 165, a VL-CDR2 comprising the amino acid sequence of SEQ ID NO: 166, and a VL-CDR3 comprising the amino acid sequence of SEQ ID NO: 167; or
p. a VH-CDR1 comprising the amino acid sequence of SEQ ID NO: 171, a VH-CDR2 comprising the amino acid sequence of SEQ ID NO: 172, a VH-CDR3 comprising the amino acid sequence SEQ ID NO: 173, a VL-CDR1 comprising the amino sequence SEQ ID NO: 175, a VL-CDR2 comprising the amino acid sequence of SEQ ID NO: 176, and a VL-CDR3 comprising the amino acid sequence of SEQ ID NO: 177; or
q. a VH-CDR1 comprising the amino acid sequence of SEQ ID NO: 181, a VH-CDR2 comprising the amino acid sequence of SEQ ID NO: 182, a VH-CDR3 comprising the amino acid sequence SEQ ID NO: 183, a VL-CDR1 comprising the amino sequence SEQ ID NO: 185, a VL-CDR2 comprising the amino acid sequence of SEQ ID NO: 186, and a VL-CDR3 comprising the amino acid sequence of SEQ ID NO: 187; or
r. a VH-CDR1 comprising the amino acid sequence of SEQ ID NO: 191, a VH-CDR2 comprising the amino acid sequence of SEQ ID NO: 192, a VH-CDR3 comprising the amino acid sequence SEQ ID NO: 193, a VL-CDR1 comprising the amino sequence SEQ ID NO: 195, a VL-CDR2 comprising the amino acid sequence of SEQ ID NO: 196, and a VL-CDR3 comprising the amino acid sequence of SEQ ID NO: 197; or
s. a VH-CDR1 comprising the amino acid sequence of SEQ ID NO: 201, a VH-CDR2 comprising the amino acid sequence of SEQ ID NO: 202, a VH-CDR3 comprising the amino acid sequence SEQ ID NO: 203, a VL-CDR1 comprising the amino sequence SEQ ID NO: 205, a VL-CDR2 comprising the amino acid sequence of SEQ ID NO: 206, and a VL-CDR3 comprising the amino acid sequence of SEQ ID NO: 207; or
t. a VH-CDR1 comprising the amino acid sequence of SEQ ID NO: 211, a VH-CDR2 comprising the amino acid sequence of SEQ ID NO: 212, a VH-CDR3 comprising the amino acid sequence SEQ ID NO: 213, a VL-CDR1 comprising the amino sequence SEQ ID NO: 215, a VL-CDR2 comprising the amino acid sequence of SEQ ID NO: 186, and a VL-CDR3 comprising the amino acid sequence of SEQ ID NO: 217; or
u. a VH-CDR1 comprising the amino acid sequence of SEQ ID NO: 221, a VH-CDR2 comprising the amino acid sequence of SEQ ID NO: 222, a VH-CDR3 comprising the amino acid sequence SEQ ID NO: 223, a VL-CDR1 comprising the amino sequence SEQ ID NO: 225, a VL-CDR2 comprising the amino acid sequence of SEQ ID NO: 226, and a VL-CDR3 comprising the amino acid sequence of SEQ ID NO: 227; or
v. a VH-CDR1 comprising the amino acid sequence of SEQ ID NO: 231, a VH-CDR2 comprising the amino acid sequence of SEQ ID NO: 232, a VH-CDR3 comprising the amino acid sequence SEQ ID NO: 233, a VL-CDR1 comprising the amino sequence SEQ ID NO: 235, a VL-CDR2 comprising the amino acid sequence of SEQ ID NO: 236, and a VL-CDR3 comprising the amino acid sequence of SEQ ID NO: 237; or
w. a VH-CDR1 comprising the amino acid sequence of SEQ ID NO: 241, a VH-CDR2 comprising the amino acid sequence of SEQ ID NO: 152, a VH-CDR3 comprising the amino acid sequence SEQ ID NO: 243, a VL-CDR1 comprising the amino sequence SEQ ID NO: 245, a VL-CDR2 comprising the amino acid sequence of SEQ ID NO: 246, and a VL-CDR3 comprising the amino acid sequence of SEQ ID NO: 247; or
x. a VH-CDR1 comprising the amino acid sequence of SEQ ID NO: 251, a VH-CDR2 comprising the amino acid sequence of SEQ ID NO: 252, a VH-CDR3 comprising the amino acid sequence SEQ ID NO: 253, a VL-CDR1 comprising the amino sequence SEQ ID NO: 255, a VL-CDR2 comprising the amino acid sequence of SEQ ID NO: 256, and a VL-CDR3 comprising the amino acid sequence of SEQ ID NO: 257; or
y. a VH-CDR1 comprising the amino acid sequence of SEQ ID NO: 261, a VH-CDR2 comprising the amino acid sequence of SEQ ID NO: 262, a VH-CDR3 comprising the amino acid sequence SEQ ID NO: 263, a VL-CDR1 comprising the amino sequence SEQ ID NO: 265, a VL-CDR2 comprising the amino acid sequence of SEQ ID NO: 266, and a VL-CDR3 comprising the amino acid sequence of SEQ ID NO: 267; or
z. a VH-CDR1 comprising the amino acid sequence of SEQ ID NO: 271, a VH-CDR2 comprising the amino acid sequence of SEQ ID NO: 272, a VH-CDR3 comprising the amino acid sequence SEQ ID NO: 203, a VL-CDR1 comprising the amino sequence SEQ ID NO: 275, a VL-CDR2 comprising the amino acid sequence of SEQ ID NO: 276, and a VL-CDR3 comprising the amino acid sequence of SEQ ID NO: 207; or
aa. a VH-CDR1 comprising the amino acid sequence of SEQ ID NO: 281, a VH-CDR2 comprising the amino acid sequence of SEQ ID NO: 152, a VH-CDR3 comprising the amino acid sequence SEQ ID NO: 283, a VL-CDR1 comprising the amino sequence SEQ ID NO: 285, a VL-CDR2 comprising the amino acid sequence of SEQ ID NO: 286, and a VL-CDR3 comprising the amino acid sequence of SEQ ID NO: 287; or
bb. a VH-CDR1 comprising the amino acid sequence of SEQ ID NO: 291, a VH-CDR2 comprising the amino acid sequence of SEQ ID NO: 292, a VH-CDR3 comprising the amino acid sequence SEQ ID NO: 293, a VL-CDR1 comprising the amino sequence SEQ ID NO: 295, a VL-CDR2 comprising the amino acid sequence of SEQ ID NO: 26, and a VL-CDR3 comprising the amino acid sequence of SEQ ID NO: 297; or
cc. a VH-CDR1 comprising the amino acid sequence of SEQ ID NO: 71, a VH-CDR2 comprising the amino acid sequence of SEQ ID NO: 302, a VH-CDR3 comprising the amino acid sequence SEQ ID NO: 303, a VL-CDR1 comprising the amino sequence SEQ ID NO: 305, a VL-CDR2 comprising the amino acid sequence of SEQ ID NO: 126, and a VL-CDR3 comprising the amino acid sequence of SEQ ID NO: 307; or
dd. a VH-CDR1 comprising the amino acid sequence of SEQ ID NO: 161, a VH-CDR2 comprising the amino acid sequence of SEQ ID NO: 312, a VH-CDR3 comprising the amino acid sequence RDY (Arg-Asp-Tyr), a VL-CDR1 comprising the amino sequence SEQ ID NO: 315, a VL-CDR2 comprising the amino acid sequence of SEQ ID NO: 186, and a VL-CDR3 comprising the amino acid sequence of SEQ ID NO: 317; or
ee. a VH-CDR1 comprising the amino acid sequence of SEQ ID NO: 161, a VH-CDR2 comprising the amino acid sequence of SEQ ID NO: 322, a VH-CDR3 comprising the amino acid sequence SEQ ID NO: 323, a VL-CDR1 comprising the amino sequence SEQ ID NO: 205, a VL-CDR2 comprising the amino acid sequence of SEQ ID NO: 326, and a VL-CDR3 comprising the amino acid sequence of SEQ ID NO: 327; or
ff. a VH-CDR1 comprising the amino acid sequence of SEQ ID NO: 331, a VH-CDR2 comprising the amino acid sequence of SEQ ID NO: 332, a VH-CDR3 comprising the amino acid sequence SEQ ID NO: 333, a VL-CDR1 comprising the amino sequence SEQ ID NO: 335, a VL-CDR2 comprising the amino acid sequence of SEQ ID NO: 336, and a VL-CDR3 comprising the amino acid sequence of SEQ ID NO: 337; or
gg. a VH-CDR1 comprising the amino acid sequence of SEQ ID NO: 161, a VH-CDR2 comprising the amino acid sequence of SEQ ID NO: 342, a VH-CDR3 comprising the amino acid sequence SEQ ID NO: 323, a VL-CDR1 comprising the amino sequence SEQ ID NO: 205, a VL-CDR2 comprising the amino acid sequence of SEQ ID NO: 326, and a VL-CDR3 comprising the amino acid sequence of SEQ ID NO: 347; or
hh. a VH-CDR1 comprising the amino acid sequence of SEQ ID NO: 331, a VH-CDR2 comprising the amino acid sequence of SEQ ID NO: 352, a VH-CDR3 comprising the amino acid sequence SEQ ID NO: 353, a VL-CDR1 comprising the amino sequence SEQ ID NO: 205, a VL-CDR2 comprising the amino acid sequence of SEQ ID NO: 336, and a VL-CDR3 comprising the amino acid sequence of SEQ ID NO: 337; or
ii. a VH-CDR1 comprising the amino acid sequence of SEQ ID NO: 361, a VH-CDR2 comprising the amino acid sequence of SEQ ID NO: 362, a VH-CDR3 comprising the amino acid sequence RDY (Arg-Asp-Tyr), a VL-CDR1 comprising the amino sequence SEQ ID NO: 315, a VL-CDR2 comprising the amino acid sequence of SEQ ID NO: 186, and a VL-CDR3 comprising the amino acid sequence of SEQ ID NO: 367; or
jj. a VH-CDR1 comprising the amino acid sequence of SEQ ID NO: 371, a VH-CDR2 comprising the amino acid sequence of SEQ ID NO: 372, a VH-CDR3 comprising the amino acid sequence SEQ ID NO: 373, a VL-CDR1 comprising the amino sequence SEQ ID NO: 375, a VL-CDR2 comprising the amino acid sequence of SEQ ID NO: 376, and a VL-CDR3 comprising the amino acid sequence of SEQ ID NO: 377; or
kk. a VH-CDR1 comprising the amino acid sequence of SEQ ID NO: 381, a VH-CDR2 comprising the amino acid sequence of SEQ ID NO: 382, a VH-CDR3 comprising the amino acid sequence SEQ ID NO: 383, a VL-CDR1 comprising the amino sequence SEQ ID NO: 385, a VL-CDR2 comprising the amino acid sequence of SEQ ID NO: 386, and a VL-CDR3 comprising the amino acid sequence of SEQ ID NO: 387; or
ll. a VH-CDR1 comprising the amino acid sequence of SEQ ID NO: 271, a VH-CDR2 comprising the amino acid sequence of SEQ ID NO: 392, a VH-CDR3 comprising the amino acid sequence SEQ ID NO: 393, a VL-CDR1 comprising the amino sequence SEQ ID NO: 335, a VL-CDR2 comprising the amino acid sequence of SEQ ID NO: 276, and a VL-CDR3 comprising the amino acid sequence of SEQ ID NO: 207.

In one embodiment, an ASC binding molecule, particularly an anti-ASC antibody or an antigen-binding fragment thereof of the invention comprises a Heavy Chain Variable Region comprising:
a. a VH-CDR1 comprising the amino acid sequence of SEQ ID NO: 11, a VH-CDR2 comprising the amino acid sequence of SEQ ID NO: 12, and a VH-CDR3 comprising the amino acid sequence NEV (Asn-Glu-Val); or
b. a VH-CDR1 comprising the amino acid sequence of SEQ ID NO: 21, a VH-CDR2 comprising the amino acid sequence of SEQ ID NO: 22, a VH-CDR3 comprising the amino acid sequence SEQ ID NO: 23; or
c. a VH-CDR1 comprising the amino acid sequence of SEQ ID NO: 31, a VH-CDR2 comprising the amino acid sequence of SEQ ID NO: 32, a VH-CDR3 comprising the amino acid sequence of SEQ ID NO: 33; or
d. a VH-CDR1 comprising the amino acid sequence of SEQ ID NO: 41, a VH-CDR2 comprising the amino acid sequence of SEQ ID NO: 42, a VH-CDR3 comprising the amino acid sequence SEQ ID NO: 43; or
e. a VH-CDR1 comprising the amino acid sequence of SEQ ID NO: 51, a VH-CDR2 comprising the amino acid sequence of SEQ ID NO: 52, a VH-CDR3 comprising the amino acid sequence SEQ ID NO: 53; or
f. a VH-CDR1 comprising the amino acid sequence of SEQ ID NO: 61, a VH-CDR2 comprising the amino acid sequence of SEQ ID NO: 62, a VH-CDR3 comprising the amino acid sequence SEQ ID NO: 63; or
g. a VH-CDR1 comprising the amino acid sequence of SEQ ID NO: 71, a VH-CDR2 comprising the amino acid sequence of SEQ ID NO: 72, a VH-CDR3 comprising the amino acid sequence SEQ ID NO: 73; or
h. a VH-CDR1 comprising the amino acid sequence of SEQ ID NO: 81, a VH-CDR2 comprising the amino acid sequence of SEQ ID NO: 82, a VH-CDR3 comprising the amino acid sequence SEQ ID NO: 83; or
i. a VH-CDR1 comprising the amino acid sequence of SEQ ID NO: 91, a VH-CDR2 comprising the amino acid sequence of SEQ ID NO: 92, a VH-CDR3 comprising the amino acid sequence SEQ ID NO: 93; or
j. a VH-CDR1 comprising the amino acid sequence of SEQ ID NO: 111, a VH-CDR2 comprising the amino acid sequence of SEQ ID NO: 112, a VH-CDR3 comprising the amino acid sequence SEQ ID NO: 113; or
k. a VH-CDR1 comprising the amino acid sequence of SEQ ID NO: 121, a VH-CDR2 comprising the amino acid sequence of SEQ ID NO: 122, a VH-CDR3 comprising the amino acid sequence SEQ ID NO: 123; or
l. a VH-CDR1 comprising the amino acid sequence of SEQ ID NO: 131, a VH-CDR2 comprising the amino acid sequence of SEQ ID NO: 132, a VH-CDR3 comprising the amino acid sequence SEQ ID NO: 133; or
m. a VH-CDR1 comprising the amino acid sequence of SEQ ID NO: 111, a VH-CDR2 comprising the amino acid sequence of SEQ ID NO: 142, a VH-CDR3 comprising the amino acid sequence SEQ ID NO: 143; or
n. a VH-CDR1 comprising the amino acid sequence of SEQ ID NO: 151, a VH-CDR2 comprising the amino acid sequence of SEQ ID NO: 152, a VH-CDR3 comprising the amino acid sequence SEQ ID NO: 153; or
o. a VH-CDR1 comprising the amino acid sequence of SEQ ID NO: 161, a VH-CDR2 comprising the amino acid sequence of SEQ ID NO: 162, a VH-CDR3 comprising the amino acid sequence SEQ ID NO: 163; or
p. a VH-CDR1 comprising the amino acid sequence of SEQ ID NO: 171, a VH-CDR2 comprising the amino acid sequence of SEQ ID NO: 172, a VH-CDR3 comprising the amino acid sequence SEQ ID NO: 173; or
q. a VH-CDR1 comprising the amino acid sequence of SEQ ID NO: 181, a VH-CDR2 comprising the amino acid sequence of SEQ ID NO: 182, a VH-CDR3 comprising the amino acid sequence SEQ ID NO: 183; or
r. a VH-CDR1 comprising the amino acid sequence of SEQ ID NO: 191, a VH-CDR2 comprising the amino acid sequence of SEQ ID NO: 192, a VH-CDR3 comprising the amino acid sequence SEQ ID NO: 193; or
s. a VH-CDR1 comprising the amino acid sequence of SEQ ID NO: 201, a VH-CDR2 comprising the amino acid sequence of SEQ ID NO: 202, a VH-CDR3 comprising the amino acid sequence SEQ ID NO: 203; or
t. a VH-CDR1 comprising the amino acid sequence of SEQ ID NO: 211, a VH-CDR2 comprising the amino acid sequence of SEQ ID NO: 212, a VH-CDR3 comprising the amino acid sequence SEQ ID NO: 213; or
u. a VH-CDR1 comprising the amino acid sequence of SEQ ID NO: 221, a VH-CDR2 comprising the amino acid sequence of SEQ ID NO: 222, a VH-CDR3 comprising the amino acid sequence SEQ ID NO: 223; or
v. a VH-CDR1 comprising the amino acid sequence of SEQ ID NO: 231, a VH-CDR2 comprising the amino acid sequence of SEQ ID NO: 232, a VH-CDR3 comprising the amino acid sequence SEQ ID NO: 233; or
w. a VH-CDR1 comprising the amino acid sequence of SEQ ID NO: 241, a VH-CDR2 comprising the amino acid sequence of SEQ ID NO: 152, a VH-CDR3 comprising the amino acid sequence SEQ ID NO: 243; or
x. a VH-CDR1 comprising the amino acid sequence of SEQ ID NO: 251, a VH-CDR2 comprising the amino acid sequence of SEQ ID NO: 252, a VH-CDR3 comprising the amino acid sequence SEQ ID NO: 253; or
y. a VH-CDR1 comprising the amino acid sequence of SEQ ID NO: 261, a VH-CDR2 comprising the amino acid sequence of SEQ ID NO: 262, a VH-CDR3 comprising the amino acid sequence SEQ ID NO: 263; or
z. a VH-CDR1 comprising the amino acid sequence of SEQ ID NO: 271, a VH-CDR2 comprising the amino acid sequence of SEQ ID NO: 272, a VH-CDR3 comprising the amino acid sequence SEQ ID NO: 203; or
aa. a VH-CDR1 comprising the amino acid sequence of SEQ ID NO: 281, a VH-CDR2 comprising the amino acid sequence of SEQ ID NO: 152; a VH-CDR3 comprising the amino acid sequence of SEQ ID NO: 283
bb. a VH-CDR1 comprising the amino acid sequence of SEQ ID NO: 291, a VH-CDR2 comprising the amino acid sequence of SEQ ID NO: 292, a VH-CDR3 comprising the amino acid sequence SEQ ID NO: 293; or
cc. a VH-CDR1 comprising the amino acid sequence of SEQ ID NO: 71, a VH-CDR2 comprising the amino acid sequence of SEQ ID NO: 302, a VH-CDR3 comprising the amino acid sequence SEQ ID NO: 303; or
dd. a VH-CDR1 comprising the amino acid sequence of SEQ ID NO: 161, a VH-CDR2 comprising the amino acid sequence of SEQ ID NO: 312, a VH-CDR3 comprising the amino acid sequence RDY (Arg-Asp-Tyr); or
ee. a VH-CDR1 comprising the amino acid sequence of SEQ ID NO: 161, a VH-CDR2 comprising the amino acid sequence of SEQ ID NO: 322, a VH-CDR3 comprising the amino acid sequence SEQ ID NO: 323; or
ff. a VH-CDR1 comprising the amino acid sequence of SEQ ID NO: 331, a VH-CDR2 comprising the amino acid sequence of SEQ ID NO: 332, a VH-CDR3 comprising the amino acid sequence SEQ ID NO: 333; or
gg. a VH-CDR1 comprising the amino acid sequence of SEQ ID NO: 161, a VH-CDR2 comprising the amino acid sequence of SEQ ID NO: 342, a VH-CDR3 comprising the amino acid sequence SEQ ID NO: 323; or
hh. a VH-CDR1 comprising the amino acid sequence of SEQ ID NO: 331, a VH-CDR2 comprising the amino acid sequence of SEQ ID NO: 352, a VH-CDR3 comprising the amino acid sequence SEQ ID NO: 353; or
ii. a VH-CDR1 comprising the amino acid sequence of SEQ ID NO: 361, a VH-CDR2 comprising the amino acid sequence of SEQ ID NO: 362, a VH-CDR3 comprising the amino acid sequence RDY (Arg-Asp-Tyr); or
jj. a VH-CDR1 comprising the amino acid sequence of SEQ ID NO: 371, a VH-CDR2 comprising the amino acid sequence of SEQ ID NO: 372, a VH-CDR3 comprising the amino acid sequence SEQ ID NO: 373; or
kk. a VH-CDR1 comprising the amino acid sequence of SEQ ID NO: 381, a VH-CDR2 comprising the amino acid sequence of SEQ ID NO: 382, a VH-CDR3 comprising the amino acid sequence SEQ ID NO: 383; or
ll. a VH-CDR1 comprising the amino acid sequence of SEQ ID NO: 271, a VH-CDR2 comprising the amino acid sequence of SEQ ID NO: 392, a VH-CDR3 comprising the amino acid sequence SEQ ID NO: 393.

In one embodiment, an ASC binding molecule, particularly an anti-ASC antibody or an antigen-binding fragment thereof of the invention comprises a Light Chain Variable Region (VL) which comprises:
a. a VL-CDR1 comprising the amino sequence SEQ ID NO: 15, a VL-CDR2 comprising the amino acid sequence of SEQ ID NO: 16, and a VL-CDR3 comprising the amino acid sequence of SEQ ID NO: 17; or
b. a VL-CDR1 comprising the amino sequence SEQ ID NO: 25, a VL-CDR2 comprising the amino acid sequence of SEQ ID NO: 26, and a VL-CDR3 comprising the amino acid sequence of SEQ ID NO: 27; or
c. a VL-CDR1 comprising the amino sequence SEQ ID NO: 35, a VL-CDR2 comprising the amino acid sequence of SEQ ID NO: 36, and a VL-CDR3 comprising the amino acid sequence of SEQ ID NO: 37; or
d. a VL-CDR1 comprising the amino sequence SEQ ID NO: 45, a VL-CDR2 comprising the amino acid sequence of SEQ ID NO: 46, and a VL-CDR3 comprising the amino acid sequence of SEQ ID NO: 47; or
e. a VL-CDR1 comprising the amino sequence SEQ ID NO: 55, a VL-CDR2 comprising the amino acid sequence of SEQ ID NO: 56, and a VL-CDR3 comprising the amino acid sequence of SEQ ID NO: 57; or
f. a VL-CDR1 comprising the amino sequence SEQ ID NO: 65, a VL-CDR2 comprising the amino acid sequence of SEQ ID NO: 66, and a VL-CDR3 comprising the amino acid sequence of SEQ ID NO: 67; or
g. a VL-CDR1 comprising the amino sequence SEQ ID NO: 75, a VL-CDR2 comprising the amino acid sequence of SEQ ID NO: 76, and a VL-CDR3 comprising the amino acid sequence of SEQ ID NO: 77; or
h. a VL-CDR1 comprising the amino sequence SEQ ID NO: 85, a VL-CDR2 comprising the amino acid sequence of SEQ ID NO: 86, and a VL-CDR3 comprising the amino acid sequence of SEQ ID NO: 87; or
i. a VL-CDR1 comprising the amino sequence SEQ ID NO: 95, a VL-CDR2 comprising the amino acid sequence of SEQ ID NO: 96, and a VL-CDR3 comprising the amino acid sequence of SEQ ID NO: 97; or
j. a VL-CDR1 comprising the amino sequence SEQ ID NO: 115, a VL-CDR2 comprising the amino acid sequence of SEQ ID NO: 116, and a VL-CDR3 comprising the amino acid sequence of SEQ ID NO: 117; or
k. VL-CDR1 comprising the amino sequence SEQ ID NO: 125, a VL-CDR2 comprising the amino acid sequence of SEQ ID NO: 126, and a VL-CDR3 comprising the amino acid sequence of SEQ ID NO: 127; or
l. a VL-CDR1 comprising the amino sequence SEQ ID NO: 135, a VL-CDR2 comprising the amino acid sequence of SEQ ID NO: 66, and a VL-CDR3 comprising the amino acid sequence of SEQ ID NO: 137; or
m. a VL-CDR1 comprising the amino sequence SEQ ID NO: 145, a VL-CDR2 comprising the amino acid sequence of SEQ ID NO: 66, and a VL-CDR3 comprising the amino acid sequence of SEQ ID NO: 147; or
n. a VL-CDR1 comprising the amino sequence SEQ ID NO: 155, a VL-CDR2 comprising the amino acid sequence of SEQ ID NO: 156, and a VL-CDR3 comprising the amino acid sequence of SEQ ID NO: 157; or
o. a VL-CDR1 comprising the amino sequence SEQ ID NO: 165, a VL-CDR2 comprising the amino acid sequence of SEQ ID NO: 166, and a VL-CDR3 comprising the amino acid sequence of SEQ ID NO: 167; or
p. a VL-CDR1 comprising the amino sequence SEQ ID NO: 175, a VL-CDR2 comprising the amino acid sequence of SEQ ID NO: 176, and a VL-CDR3 comprising the amino acid sequence of SEQ ID NO: 177; or
q. a VL-CDR1 comprising the amino sequence SEQ ID NO: 185, a VL-CDR2 comprising the amino acid sequence of SEQ ID NO: 186, and a VL-CDR3 comprising the amino acid sequence of SEQ ID NO: 187; or
r. a VL-CDR1 comprising the amino sequence SEQ ID NO: 195, a VL-CDR2 comprising the amino acid sequence of SEQ ID NO: 196, and a VL-CDR3 comprising the amino acid sequence of SEQ ID NO: 197; or
s. a VL-CDR1 comprising the amino sequence SEQ ID NO: 205, a VL-CDR2 comprising the amino acid sequence of SEQ ID NO: 206, and a VL-CDR3 comprising the amino acid sequence of SEQ ID NO: 207; or
t. a VL-CDR1 comprising the amino sequence SEQ ID NO: 215, a VL-CDR2 comprising the amino acid sequence of SEQ ID NO: 186, and a VL-CDR3 comprising the amino acid sequence of SEQ ID NO: 217; or
u. a VL-CDR1 comprising the amino sequence SEQ ID NO: 225, a VL-CDR2 comprising the amino acid sequence of SEQ ID NO: 226, and a VL-CDR3 comprising the amino acid sequence of SEQ ID NO: 227; or
v. a VL-CDR1 comprising the amino sequence SEQ ID NO: 235, a VL-CDR2 comprising the amino acid sequence of SEQ ID NO: 236, and a VL-CDR3 comprising the amino acid sequence of SEQ ID NO: 237; or
w. a VL-CDR1 comprising the amino sequence SEQ ID NO: 245, a VL-CDR2 comprising the amino acid sequence of SEQ ID NO: 246, and a VL-CDR3 comprising the amino acid sequence of SEQ ID NO: 247; or
x. a VL-CDR1 comprising the amino sequence SEQ ID NO: 255, a VL-CDR2 comprising the amino acid sequence of SEQ ID NO: 256, and a VL-CDR3 comprising the amino acid sequence of SEQ ID NO: 257; or
y. a VL-CDR1 comprising the amino sequence SEQ ID NO: 265, a VL-CDR2 comprising the amino acid sequence of SEQ ID NO: 266, and a VL-CDR3 comprising the amino acid sequence of SEQ ID NO: 267; or
z. a VL-CDR1 comprising the amino sequence SEQ ID NO: 275, a VL-CDR2 comprising the amino acid sequence of SEQ ID NO: 276, and a VL-CDR3 comprising the amino acid sequence of SEQ ID NO: 207; or
aa. a VL-CDR1 comprising the amino sequence SEQ ID NO: 285, a VL-CDR2 comprising the amino acid sequence of SEQ ID NO: 286, and a VL-CDR3 comprising the amino acid sequence of SEQ ID NO: 287; or
bb. a VL-CDR1 comprising the amino sequence SEQ ID NO: 295, a VL-CDR2 comprising the amino acid sequence of SEQ ID NO: 26, and a VL-CDR3 comprising the amino acid sequence of SEQ ID NO: 297; or
cc. a VL-CDR1 comprising the amino sequence SEQ ID NO: 305, a VL-CDR2 comprising the amino acid sequence of SEQ ID NO: 126, and a VL-CDR3 comprising the amino acid sequence of SEQ ID NO: 307; or
dd. a VL-CDR1 comprising the amino sequence SEQ ID NO: 315, a VL-CDR2 comprising the amino acid sequence of SEQ ID NO: 186, and a VL-CDR3 comprising the amino acid sequence of SEQ ID NO: 317; or
ee. a VL-CDR1 comprising the amino sequence SEQ ID NO: 205, a VL-CDR2 comprising the amino acid sequence of SEQ ID NO: 326, and a VL-CDR3 comprising the amino acid sequence of SEQ ID NO: 327; or
ff. a VL-CDR1 comprising the amino sequence SEQ ID NO: 335, a VL-CDR2 comprising the amino acid sequence of SEQ ID NO: 336, and a VL-CDR3 comprising the amino acid sequence of SEQ ID NO: 337; or
gg. a VL-CDR1 comprising the amino sequence SEQ ID NO: 205, a VL-CDR2 comprising the amino acid sequence of SEQ ID NO: 326, and a VL-CDR3 comprising the amino acid sequence of SEQ ID NO: 347; or
hh. a VL-CDR1 comprising the amino sequence SEQ ID NO: 205, a VL-CDR2 comprising the amino acid sequence of SEQ ID NO: 336, and a VL-CDR3 comprising the amino acid sequence of SEQ ID NO: 337; or
ii. a VL-CDR1 comprising the amino sequence SEQ ID NO: 315, a VL-CDR2 comprising the amino acid sequence of SEQ ID NO: 186, and a VL-CDR3 comprising the amino acid sequence of SEQ ID NO: 367; or
jj. a VL-CDR1 comprising the amino sequence SEQ ID NO: 375, a VL-CDR2 comprising the amino acid sequence of SEQ ID NO: 376, and a VL-CDR3 comprising the amino acid sequence of SEQ ID NO: 377; or
kk. a VL-CDR1 comprising the amino sequence SEQ ID NO: 385, a VL-CDR2 comprising the amino acid sequence of SEQ ID NO: 386, and a VL-CDR3 comprising the amino acid sequence of SEQ ID NO: 387; or
ll. a VL-CDR1 comprising the amino sequence SEQ ID NO: 335, a VL-CDR2 comprising the amino acid sequence of SEQ ID NO: 276, and a VL-CDR3 comprising the amino acid sequence of SEQ ID NO: 207.

In an embodiment of the invention, an ASC binding molecule, in particular an anti-ASC antibody or an antigen-binding fragment thereof of the invention, comprises:
a. a Heavy Chain Variable Region (VH) comprising the amino acid sequence of SEQ ID NO: or a Heavy Chain Variable Region (VH) having at least 92%, 93%, 94%, 95%, 96%, 97%, 98% or 99% sequence identity to the amino acid sequence of SEQ ID NO: 10; and a Light Chain Variable Region (VL) comprising the amino acid sequence of SEQ ID NO: 14 or a Light Chain Variable Region (VL) having at least 98% or 99% sequence identity to the amino acid sequence of SEQ ID NO: 14; or
b. a Heavy Chain Variable Region (VH) comprising the amino acid sequence of SEQ ID NO: or a Heavy Chain Variable Region (VH) having at least 90%, 91%, 92%, 93%, 94%, 95%, 96%, 97%, 98% or 99% sequence identity to the amino acid sequence of SEQ ID NO: 20; and a Light Chain Variable Region (VL) comprising the amino acid sequence of SEQ ID NO: 24 or a Light Chain Variable Region (VL) having at least 95%, 96%, 97%, 98% or 99% sequence identity to the amino acid sequence of SEQ ID NO: 24; or
c. a Heavy Chain Variable Region (VH) comprising the amino acid sequence of SEQ ID NO: or a Heavy Chain Variable Region (VH) having at least 85%, 86%, 87%, 88%, 89%, 90%, 91%, 92%, 93%, 94%, 95%, 96%, 97%, 98% or 99% sequence identity to the amino acid sequence of SEQ ID NO: 30; and a Light Chain Variable Region (VL) comprising the amino acid sequence of SEQ ID NO: 34 or a Light Chain Variable Region (VL) having at least 93%, 94%, 95%, 96%, 97%, 98% or 99% sequence identity to the amino acid sequence of SEQ ID NO: 34; or
d. a Heavy Chain Variable Region (VH) comprising the amino acid sequence of SEQ ID NO: or a Heavy Chain Variable Region (VH) having at least 86%, 87%, 88%, 89%, 90%, 91%, 92%, 93%, 94%, 95%, 96%, 97%, 98% or 99% sequence identity to the amino acid sequence of SEQ ID NO: 40; and a Light Chain Variable Region (VL) comprising the amino acid sequence of SEQ ID NO: 44 or a Light Chain Variable Region (VL) having at least 96%, 97%, 98% or 99% sequence identity to the amino acid sequence of SEQ ID NO: 44; or
e. a Heavy Chain Variable Region (VH) comprising the amino acid sequence of SEQ ID NO: 50 or a Heavy Chain Variable Region (VH) having at least 94%, 95%, 96%, 97%, 98% or 99% sequence identity to the amino acid sequence of SEQ ID NO: 50; and a Light Chain Variable Region (VL) comprising the amino acid sequence of SEQ ID NO: 54 or a Light Chain Variable Region (VL) having at least 96%, 97%, 98% or 99% sequence identity to the amino acid sequence of SEQ ID NO: 54; or
f. a Heavy Chain Variable Region (VH) comprising the amino acid sequence of SEQ ID NO: 60 or a Heavy Chain Variable Region (VH) having at least 85%, 86%, 87%, 88%, 95%, 96%, 97%, 98% or 99% sequence identity to the amino acid sequence of SEQ ID NO: 60; and a Light Chain Variable Region (VL) comprising the amino acid sequence of SEQ ID NO: 64; or
g. a Heavy Chain Variable Region (VH) comprising the amino acid sequence of SEQ ID NO: 70 or a Heavy Chain Variable Region (VH) having at least 90%, 91%, 92%, 93%, 94%, 95%, 96%, 97%, 98% or 99% sequence identity to the amino acid sequence of SEQ ID NO: 70; and a Light Chain Variable Region (VL) comprising the amino acid sequence of SEQ ID NO: 74 or a Light Chain Variable Region (VL) having at least 97%, 98% or 99% sequence identity to the amino acid sequence of SEQ ID NO: 74; or
h. a Heavy Chain Variable Region (VH) comprising the amino acid sequence of SEQ ID NO: 80 or a Heavy Chain Variable Region (VH) having at least 93%, 94%, 95%, 96%, 97%, 98% or 99% sequence identity to the amino acid sequence of SEQ ID NO: 80; and a Light Chain Variable Region (VL) comprising the amino acid sequence of SEQ ID NO: 84 or a Light Chain Variable Region (VL) having at least 95%, 96%, 97%, 98% or 99% sequence identity to the amino acid sequence of SEQ ID NO: 84; or
i. a Heavy Chain Variable Region (VH) comprising the amino acid sequence of SEQ ID NO: 90 or a Heavy Chain Variable Region (VH) having at least 93%, 94%, 95%, 96%, 97%, 98% or 99% sequence identity to the amino acid sequence of SEQ ID NO: 90; and a Light Chain Variable Region (VL) comprising the amino acid sequence of SEQ ID NO: 94 or a Light Chain Variable Region (VL) having at least 93%, 94%, 95%, 96%, 97%, 98% or 99% sequence identity to the amino acid sequence of SEQ ID NO: 94; or
j. a Heavy Chain Variable Region (VH) comprising the amino acid sequence of SEQ ID NO: 110 or a Heavy Chain Variable Region (VH) having at least 88%, 89%, 90%, 91%, 92%, 93%, 94%, 95%, 96%, 97%, 98% or 99% sequence identity to the amino acid sequence of SEQ ID NO: 110; and a Light Chain Variable Region (VL) comprising the amino acid sequence of SEQ ID NO: 114 or a Light Chain Variable Region (VL) having at least 98% or 99% sequence identity to the amino acid sequence of SEQ ID NO: 114; or
k. a Heavy Chain Variable Region (VH) comprising the amino acid sequence of SEQ ID NO: 120 or a Heavy Chain Variable Region (VH) having at least 92%, 93%, 94%, 95%, 96%, 97%, 98% or 99% sequence identity to the amino acid sequence of SEQ ID NO: 120; and a Light Chain Variable Region (VL) comprising the amino acid sequence of SEQ ID NO: 124 or a Light Chain Variable Region (VL) having at least 98% or 99% sequence identity to the amino acid sequence of SEQ ID NO: 124; or
l. a Heavy Chain Variable Region (VH) comprising the amino acid sequence of SEQ ID NO: 130 or a Heavy Chain Variable Region (VH) having at least 87%, 88%, 89%, 90%, 91%, 92%, 93%, 94%, 95%, 96%, 97%, 98% or 99% sequence identity to the amino acid sequence of SEQ ID NO: 130; and a Light Chain Variable Region (VL) comprising the amino acid sequence of SEQ ID NO: 134 or a Light Chain Variable Region (VL) having at least 98% or 99% sequence identity to the amino acid sequence of SEQ ID NO: 134; or
m. a Heavy Chain Variable Region (VH) comprising the amino acid sequence of SEQ ID NO: 140 or a Heavy Chain Variable Region (VH) having at least 94%, 95%, 96%, 97%, 98% or 99% sequence identity to the amino acid sequence of SEQ ID NO: 140; and a Light Chain Variable Region (VL) comprising the amino acid sequence of SEQ ID NO: 144 or a Light Chain Variable Region (VL) having at least 98% or 99% sequence identity to the amino acid sequence of SEQ ID NO: 144; or
n. a Heavy Chain Variable Region (VH) comprising the amino acid sequence of SEQ ID NO: 150 or a Heavy Chain Variable Region (VH) having at least 90%, 91%, 92%, 93%, 94%, 95%, 96%, 97%, 98% or 99% sequence identity to the amino acid sequence of SEQ ID NO: 150; and a Light Chain Variable Region (VL) comprising the amino acid sequence of SEQ ID NO: 154 or a Light Chain Variable Region (VL) having at least 93%, 94%, 95%, 96%, 97%, 98% or 99% sequence identity to the amino acid sequence of SEQ ID NO: 154; or
o. a Heavy Chain Variable Region (VH) comprising the amino acid sequence of SEQ ID NO: 160 or a Heavy Chain Variable Region (VH) having at least 88%, 89%, 90%, 91%, 92%, 93%, 94%, 95%, 96%, 97%, 98% or 99% sequence identity to the amino acid sequence of SEQ ID NO: 160; and a Light Chain Variable Region (VL) comprising the amino acid sequence of SEQ ID NO: 164 or a Light Chain Variable Region (VL) having at least 96%, 97%, 98% or 99% sequence identity to the amino acid sequence of SEQ ID NO: 164; or
p. a Heavy Chain Variable Region (VH) comprising the amino acid sequence of SEQ ID NO: 170 or a Heavy Chain Variable Region (VH) having at least 85%, 86%, 87%, 88%, 89%, 90%, 91%, 92%, 93%, 94%, 95%, 96%, 97%, 98% or 99% sequence identity to the amino acid sequence of SEQ ID NO: 170; and a Light Chain Variable Region (VL) comprising the amino acid sequence of SEQ ID NO: 174 or a Light Chain Variable Region (VL) having at least 93%, 94%, 95%, 96%, 97%, 98% or 99% sequence identity to the amino acid sequence of SEQ ID NO: 174; or
q. a Heavy Chain Variable Region (VH) comprising the amino acid sequence of SEQ ID NO: 180 or a Heavy Chain Variable Region (VH) having at least 94%, 95%, 96%, 97%, 98% or 99% sequence identity to the amino acid sequence of SEQ ID NO: 180; and a Light Chain Variable Region (VL) comprising the amino acid sequence of SEQ ID NO: 184 or a Light Chain Variable Region (VL) having at least 97%, 98% or 99% sequence identity to the amino acid sequence of SEQ ID NO: 184; or
r. a Heavy Chain Variable Region (VH) comprising the amino acid sequence of SEQ ID NO: 190 or a Heavy Chain Variable Region (VH) having at least 87%, 88%, 89%, 90%, 91%, 92%, 93%, 94%, 95%, 96%, 97%, 98% or 99% sequence identity to the amino acid sequence of SEQ ID NO: 190; and a Light Chain Variable Region (VL) comprising the amino acid sequence of SEQ ID NO: 194; or
s. a Heavy Chain Variable Region (VH) comprising the amino acid sequence of SEQ ID NO: 200 or a Heavy Chain Variable Region (VH) having at least 93%, 94%, 95%, 96%, 97%, 98% or 99% sequence identity to the amino acid sequence of SEQ ID NO: 200; and a Light Chain Variable Region (VL) comprising the amino acid sequence of SEQ ID NO: 204 or a Light Chain Variable Region (VL) having at least 99% sequence identity to the amino acid sequence of SEQ ID NO: 204; or
t. a Heavy Chain Variable Region (VH) comprising the amino acid sequence of SEQ ID NO: 210 or a Heavy Chain Variable Region (VH) having at least 92%, 93%, 94%, 95%, 96%, 97%, 98% or 99% sequence identity to the amino acid sequence of SEQ ID NO: 210; and a Light Chain Variable Region (VL) comprising the amino acid sequence of SEQ ID NO: 214 or a Light Chain Variable Region (VL) having at least 96%, 97%, 98% or 99% sequence identity to the amino acid sequence of SEQ ID NO: 214; or
u. a Heavy Chain Variable Region (VH) comprising the amino acid sequence of SEQ ID NO: 220 or a Heavy Chain Variable Region (VH) having at least 84%, 85%, 86%, 87%, 88%, 89%, 90%, 91%, 92%, 93%, 94%, 95%, 96%, 97%, 98% or 99% sequence identity to the amino acid sequence of SEQ ID NO: 220; and a Light Chain Variable Region (VL) comprising the amino acid sequence of SEQ ID NO: 224 or a Light Chain Variable Region (VL) having at least 93%, 94%, 95%, 96%, 97%, 98% or 99% sequence identity to the amino acid sequence of SEQ ID NO: 224; or
v. a Heavy Chain Variable Region (VH) comprising the amino acid sequence of SEQ ID NO: 230 or a Heavy Chain Variable Region (VH) having at least 84%, 85%, 86%, 87%, 88%, 89%, 90%, 91%, 92%, 93%, 94%, 95%, 96%, 97%, 98% or 99% sequence identity to the amino acid sequence of SEQ ID NO: 230; and a Light Chain Variable Region (VL) comprising the amino acid sequence of SEQ ID NO: 234 or a Light Chain Variable Region (VL) having at least 92%, 93%, 94%, 95%, 96%, 97%, 98% or 99% sequence identity to the amino acid sequence of SEQ ID NO: 234; or
w. a Heavy Chain Variable Region (VH) comprising the amino acid sequence of SEQ ID NO: 240 or a Heavy Chain Variable Region (VH) having at least 88%, 89%, 90%, 91%, 92%, 93%, 94%, 95%, 96%, 97%, 98% or 99% sequence identity to the amino acid sequence of SEQ ID NO: 240; and a Light Chain Variable Region (VL) comprising the amino acid sequence of SEQ ID NO: 244 or a Light Chain Variable Region (VL) having at least 97%, 98% or 99% sequence identity to the amino acid sequence of SEQ ID NO: 244; or
x. a Heavy Chain Variable Region (VH) comprising the amino acid sequence of SEQ ID NO: 250 or a Heavy Chain Variable Region (VH) having at least 89%, 90%, 91%, 92%, 93%, 94%, 95%, 96%, 97%, 98% or 99% sequence identity to the amino acid sequence of SEQ ID NO: 250; and a Light Chain Variable Region (VL) comprising the amino acid sequence of SEQ ID NO: 254 or a Light Chain Variable Region (VL) having at least 97%, 98% or 99% sequence identity to the amino acid sequence of SEQ ID NO: 254; or
y. a Heavy Chain Variable Region (VH) comprising the amino acid sequence of SEQ ID NO: 260 or a Heavy Chain Variable Region (VH) having at least 90%, 91%, 92%, 93%, 94%, 95%, 96%, 97%, 98% or 99% sequence identity to the amino acid sequence of SEQ ID NO: 260; and a Light Chain Variable Region (VL) comprising the amino acid sequence of SEQ ID NO: 264 or a Light Chain Variable Region (VL) having at least 97%, 98% or 99% sequence identity to the amino acid sequence of SEQ ID NO: 264; or
z. a Heavy Chain Variable Region (VH) comprising the amino acid sequence of SEQ ID NO: 270 or a Heavy Chain Variable Region (VH) having at least 91%, 92%, 93%, 94%, 95%, 96%, 97%, 98% or 99% sequence identity to the amino acid sequence of SEQ ID NO: 270; and a Light Chain Variable Region (VL) comprising the amino acid sequence of SEQ ID NO: 274 or a Light Chain Variable Region (VL) having at least or 99% sequence identity to the amino acid sequence of SEQ ID NO: 274; or
aa. a Heavy Chain Variable Region (VH) comprising the amino acid sequence of SEQ ID NO: 280 or a Heavy Chain Variable Region (VH) having at least 87%, 88%, 89%, 90%, 91%, 92%, 93%, 94%, 95%, 96%, 97%, 98% or 99% sequence identity to the amino acid sequence of SEQ ID NO: 280; and a Light Chain Variable Region (VL) comprising the amino acid sequence of SEQ ID NO: 284 or a Light Chain Variable Region (VL) having at least 98% or 99% sequence identity to the amino acid sequence of SEQ ID NO: 284; or
bb. a Heavy Chain Variable Region (VH) comprising the amino acid sequence of SEQ ID NO: 290 or a Heavy Chain Variable Region (VH) having at least 92%, 93%, 94%, 95%, 96%, 97%, 98% or 99% sequence identity to the amino acid sequence of SEQ ID NO: 290; and a Light Chain Variable Region (VL) comprising the amino acid sequence of SEQ ID NO: 294 or a Light Chain Variable Region (VL) having at least 99% sequence identity to the amino acid sequence of SEQ ID NO: 294; or
cc. a Heavy Chain Variable Region (VH) comprising the amino acid sequence of SEQ ID NO: 300 or a Heavy Chain Variable Region (VH) having at least 95%, 96%, 97%, 98% or 99% sequence identity to the amino acid sequence of SEQ ID NO: 300; and a Light Chain Variable Region (VL) comprising the amino acid sequence of SEQ ID NO: 304 or a Light Chain Variable Region (VL) having at least 98% or 99% sequence identity to the amino acid sequence of SEQ ID NO: 304; or
dd. a Heavy Chain Variable Region (VH) comprising the amino acid sequence of SEQ ID NO: 310 or a Heavy Chain Variable Region (VH) having at least 95%, 96%, 97%, 98% or 99% sequence identity to the amino acid sequence of SEQ ID NO: 310; and a Light Chain Variable Region (VL) comprising the amino acid sequence of SEQ ID NO: 314 or a Light Chain Variable Region (VL) having at least 97%, 98% or 99% sequence identity to the amino acid sequence of SEQ ID NO: 314; or
ee. a Heavy Chain Variable Region (VH) comprising the amino acid sequence of SEQ ID NO: 320 or a Heavy Chain Variable Region (VH) having at least 93%, 94%, 95%, 96%, 97%, 98% or 99% sequence identity to the amino acid sequence of SEQ ID NO: 320; and a Light Chain Variable Region (VL) comprising the amino acid sequence of SEQ ID NO: 324 or a Light Chain Variable Region (VL) having at least 94%, 95%, 96%, 97%, 98% or 99% sequence identity to the amino acid sequence of SEQ ID NO: 324; or
ff. a Heavy Chain Variable Region (VH) comprising the amino acid sequence of SEQ ID NO: 330 or a Heavy Chain Variable Region (VH) having at least 87%, 88%, 89%, 90%, 91%, 92%, 93%, 94%, 95%, 96%, 97%, 98% or 99% sequence identity to the amino acid sequence of SEQ ID NO: 330; and a Light Chain Variable Region (VL) comprising the amino acid sequence of SEQ ID NO: 334 or a Light Chain Variable Region (VL) having at least 96%, 97%, 98% or 99% sequence identity to the amino acid sequence of SEQ ID NO: 334; or
gg. a Heavy Chain Variable Region (VH) comprising the amino acid sequence of SEQ ID NO: 340 or a Heavy Chain Variable Region (VH) having at least 89%, 90%, 91%, 92%, 93%, 94%, 95%, 96%, 97%, 98% or 99% sequence identity to the amino acid sequence of SEQ ID NO: 340; and a Light Chain Variable Region (VL) comprising the amino acid sequence of SEQ ID NO: 344 or a Light Chain Variable Region (VL) having at least 89%, 90%, 91%, 92%, 93%, 94%, 95%, 96%, 97%, 98% or 99% sequence identity to the amino acid sequence of SEQ ID NO: 344; or
hh. a Heavy Chain Variable Region (VH) comprising the amino acid sequence of SEQ ID NO: 350 or a Heavy Chain Variable Region (VH) having at least 89%, 90%, 91%, 92%, 93%, 94%, 95%, 96%, 97%, 98% or 99% sequence identity to the amino acid sequence of SEQ ID NO: 350; and a Light Chain Variable Region (VL) comprising the amino acid sequence of SEQ ID NO: 354 or a Light Chain Variable Region (VL) having at least 96%, 97%, 98% or 99% sequence identity to the amino acid sequence of SEQ ID NO: 354; or
ii. a Heavy Chain Variable Region (VH) comprising the amino acid sequence of SEQ ID NO: 360 or a Heavy Chain Variable Region (VH) having at least 92%, 93%, 94%, 95%, 96%, 97%, 98% or 99% sequence identity to the amino acid sequence of SEQ ID NO: 360; and a Light Chain Variable Region (VL) comprising the amino acid sequence of SEQ ID NO: 364 or a Light Chain Variable Region (VL) having at least 98% or 99% sequence identity to the amino acid sequence of SEQ ID NO: 364; or
jj. a Heavy Chain Variable Region (VH) comprising the amino acid sequence of SEQ ID NO: 370 or a Heavy Chain Variable Region (VH) having at least 85%, 86%, 87%, 88%, 89%, 90%, 91%, 92%, 93%, 94%, 95%, 96%, 97%, 98% or 99% sequence identity to the amino acid sequence of SEQ ID NO: 370; and a Light Chain Variable Region (VL) comprising the amino acid sequence of SEQ ID NO: 374 or a Light Chain Variable Region (VL) having at least 94%, 95%, 96%, 97%, 98% or 99% sequence identity to the amino acid sequence of SEQ ID NO: 374; or
kk. a Heavy Chain Variable Region (VH) comprising the amino acid sequence of SEQ ID NO: 380 or a Heavy Chain Variable Region (VH) having at least 86%, 87%, 88%, 89%, 90%, 91%, 92%, 93%, 94%, 95%, 96%, 97%, 98% or 99% sequence identity to the amino acid sequence of SEQ ID NO: 380; and a Light Chain Variable Region (VL) comprising the amino acid sequence of SEQ ID NO: 384 or a Light Chain Variable Region (VL) having at least 93%, 94%, 95%, 96%, 97%, 98% or 99% sequence identity to the amino acid sequence of SEQ ID NO: 384; or
ll. a Heavy Chain Variable Region (VH) comprising the amino acid sequence of SEQ ID NO: 390 or a Heavy Chain Variable Region (VH) having at least 95%, 96%, 97%, 98% or 99% sequence identity to the amino acid sequence of SEQ ID NO: 390; and a Light Chain Variable Region (VL) comprising the amino acid sequence of SEQ ID NO: 394.

In an embodiment of the invention, an ASC binding molecule, in particular an anti-ASC antibody or an antigen-binding fragment thereof, comprises:
a. a Heavy Chain Variable Region (VH) comprising the amino acid sequence of SEQ ID NO: or a Heavy Chain Variable Region (VH) having at least 92%, 93%, 94%, 95%, 96%, 97%, 98% or 99% sequence identity to the amino acid sequence of SEQ ID NO: 10; and a Light Chain Variable Region (VL) comprising the amino acid sequence of SEQ ID NO: 14; or
b. a Heavy Chain Variable Region (VH) comprising the amino acid sequence of SEQ ID NO: or a Heavy Chain Variable Region (VH) having at least 90%, 91%, 92%, 93%, 94%, 95%, 96%, 97%, 98% or 99% sequence identity to the amino acid sequence of SEQ ID NO: 20; and a Light Chain Variable Region (VL) comprising the amino acid sequence of SEQ ID NO: 24; or
c. a Heavy Chain Variable Region (VH) comprising the amino acid sequence of SEQ ID NO: or a Heavy Chain Variable Region (VH) having at least 85%, 86%, 87%, 88%, 89%, 90%, 91%, 92%, 93%, 94%, 95%, 96%, 97%, 98% or 99% sequence identity to the amino acid sequence of SEQ ID NO: 30; and a Light Chain Variable Region (VL) comprising the amino acid sequence of SEQ ID NO: 34; or
d. a Heavy Chain Variable Region (VH) comprising the amino acid sequence of SEQ ID NO: or a Heavy Chain Variable Region (VH) having at least 86%, 87%, 88%, 89%, 90%, 91%, 92%, 93%, 94%, 95%, 96%, 97%, 98% or 99% sequence identity to the amino acid sequence of SEQ ID NO: 40; and a Light Chain Variable Region (VL) comprising the amino acid sequence of SEQ ID NO: 44; or
e. a Heavy Chain Variable Region (VH) comprising the amino acid sequence of SEQ ID NO: 50 or a Heavy Chain Variable Region (VH) having at least 94%, 95%, 96%, 97%, 98% or 99% sequence identity to the amino acid sequence of SEQ ID NO: 50; and a Light Chain Variable Region (VL) comprising the amino acid sequence of SEQ ID NO: 54; or
f. a Heavy Chain Variable Region (VH) comprising the amino acid sequence of SEQ ID NO: 60 or a Heavy Chain Variable Region (VH) having at least 85%, 86%, 87%, 88%, 95%, 96%, 97%, 98% or 99% sequence identity to the amino acid sequence of SEQ ID NO: 60; and a Light Chain Variable Region (VL) comprising the amino acid sequence of SEQ ID NO: 64; or
g. a Heavy Chain Variable Region (VH) comprising the amino acid sequence of SEQ ID NO: 70 or a Heavy Chain Variable Region (VH) having at least 90%, 91%, 92%, 93%, 94%, 95%, 96%, 97%, 98% or 99% sequence identity to the amino acid sequence of SEQ ID NO: 70; and a Light Chain Variable Region (VL) comprising the amino acid sequence of SEQ ID NO: 74; or
h. a Heavy Chain Variable Region (VH) comprising the amino acid sequence of SEQ ID NO: 80 or a Heavy Chain Variable Region (VH) having at least 93%, 94%, 95%, 96%, 97%, 98% or 99% sequence identity to the amino acid sequence of SEQ ID NO: 80; and a Light Chain Variable Region (VL) comprising the amino acid sequence of SEQ ID NO: 84; or
i. a Heavy Chain Variable Region (VH) comprising the amino acid sequence of SEQ ID NO: 90 or a Heavy Chain Variable Region (VH) having at least 93%, 94%, 95%, 96%, 97%, 98% or 99% sequence identity to the amino acid sequence of SEQ ID NO: 90; and a Light Chain Variable Region (VL) comprising the amino acid sequence of SEQ ID NO: 94; or
j. a Heavy Chain Variable Region (VH) comprising the amino acid sequence of SEQ ID NO: 110 or a Heavy Chain Variable Region (VH) having at least 88%, 89%, 90%, 91%, 92%, 93%, 94%, 95%, 96%, 97%, 98% or 99% sequence identity to the amino acid sequence of SEQ ID NO: 110; and a Light Chain Variable Region (VL) comprising the amino acid sequence of SEQ ID NO: 114; or
k. a Heavy Chain Variable Region (VH) comprising the amino acid sequence of SEQ ID NO: 120 or a Heavy Chain Variable Region (VH) having at least 92%, 93%, 94%, 95%, 96%, 97%, 98% or 99% sequence identity to the amino acid sequence of SEQ ID NO: 120; and a Light Chain Variable Region (VL) comprising the amino acid sequence of SEQ ID NO: 124; or
l. a Heavy Chain Variable Region (VH) comprising the amino acid sequence of SEQ ID NO: 130 or a Heavy Chain Variable Region (VH) having at least 87%, 88%, 89%, 90%, 91%, 92%, 93%, 94%, 95%, 96%, 97%, 98% or 99% sequence identity to the amino acid sequence of SEQ ID NO: 130; and a Light Chain Variable Region (VL) comprising the amino acid sequence of SEQ ID NO: 134; or
m. a Heavy Chain Variable Region (VH) comprising the amino acid sequence of SEQ ID NO: 140 or a Heavy Chain Variable Region (VH) having at least 94%, 95%, 96%, 97%, 98% or 99% sequence identity to the amino acid sequence of SEQ ID NO: 140; and a Light Chain Variable Region (VL) comprising the amino acid sequence of SEQ ID NO: 144; or
n. a Heavy Chain Variable Region (VH) comprising the amino acid sequence of SEQ ID NO: 150 or a Heavy Chain Variable Region (VH) having at least 90%, 91%, 92%, 93%, 94%, 95%, 96%, 97%, 98% or 99% sequence identity to the amino acid sequence of SEQ ID NO: 150; and a Light Chain Variable Region (VL) comprising the amino acid sequence of SEQ ID NO: 154; or
o. a Heavy Chain Variable Region (VH) comprising the amino acid sequence of SEQ ID NO: 160 or a Heavy Chain Variable Region (VH) having at least 88%, 89%, 90%, 91%, 92%, 93%, 94%, 95%, 96%, 97%, 98% or 99% sequence identity to the amino acid sequence of SEQ ID NO: 160; and a Light Chain Variable Region (VL) comprising the amino acid sequence of SEQ ID NO: 164; or
p. a Heavy Chain Variable Region (VH) comprising the amino acid sequence of SEQ ID NO: 170 or a Heavy Chain Variable Region (VH) having at least 85%, 86%, 87%, 88%, 89%, 90%, 91%, 92%, 93%, 94%, 95%, 96%, 97%, 98% or 99% sequence identity to the amino acid sequence of SEQ ID NO: 170; and a Light Chain Variable Region (VL) comprising the amino acid sequence of SEQ ID NO: 174; or
q. a Heavy Chain Variable Region (VH) comprising the amino acid sequence of SEQ ID NO: 180 or a Heavy Chain Variable Region (VH) having at least 94%, 95%, 96%, 97%, 98% or 99% sequence identity to the amino acid sequence of SEQ ID NO: 180; and a Light Chain Variable Region (VL) comprising the amino acid sequence of SEQ ID NO: 184; or
r. a Heavy Chain Variable Region (VH) comprising the amino acid sequence of SEQ ID NO: 190 or a Heavy Chain Variable Region (VH) having at least 87%, 88%, 89%, 90%, 91%, 92%, 93%, 94%, 95%, 96%, 97%, 98% or 99% sequence identity to the amino acid sequence of SEQ ID NO: 190; and a Light Chain Variable Region (VL) comprising the amino acid sequence of SEQ ID NO: 194; or
s. a Heavy Chain Variable Region (VH) comprising the amino acid sequence of SEQ ID NO: 200 or a Heavy Chain Variable Region (VH) having at least 93%, 94%, 95%, 96%, 97%, 98% or 99% sequence identity to the amino acid sequence of SEQ ID NO: 200; and a Light Chain Variable Region (VL) comprising the amino acid sequence of SEQ ID NO: 204; or
t. a Heavy Chain Variable Region (VH) comprising the amino acid sequence of SEQ ID NO: 210 or a Heavy Chain Variable Region (VH) having at least 92%, 93%, 94%, 95%, 96%, 97%, 98% or 99% sequence identity to the amino acid sequence of SEQ ID NO: 210; and a Light Chain Variable Region (VL) comprising the amino acid sequence of SEQ ID NO: 214; or
u. a Heavy Chain Variable Region (VH) comprising the amino acid sequence of SEQ ID NO: 220 or a Heavy Chain Variable Region (VH) having at least 84%, 85%, 86%, 87%, 88%, 89%, 90%, 91%, 92%, 93%, 94%, 95%, 96%, 97%, 98% or 99% sequence identity to the amino acid sequence of SEQ ID NO: 220; and a Light Chain Variable Region (VL) comprising the amino acid sequence of SEQ ID NO: 224; or
v. a Heavy Chain Variable Region (VH) comprising the amino acid sequence of SEQ ID NO: 230 or a Heavy Chain Variable Region (VH) having at least 84%, 85%, 86%, 87%, 88%, 89%, 90%, 91%, 92%, 93%, 94%, 95%, 96%, 97%, 98% or 99% sequence identity to the amino acid sequence of SEQ ID NO: 230; and a Light Chain Variable Region (VL) comprising the amino acid sequence of SEQ ID NO: 234; or
w. a Heavy Chain Variable Region (VH) comprising the amino acid sequence of SEQ ID NO: 240 or a Heavy Chain Variable Region (VH) having at least 88%, 89%, 90%, 91%, 92%, 93%, 94%, 95%, 96%, 97%, 98% or 99% sequence identity to the amino acid sequence of SEQ ID NO: 240; and a Light Chain Variable Region (VL) comprising the amino acid sequence of SEQ ID NO: 244; or
x. a Heavy Chain Variable Region (VH) comprising the amino acid sequence of SEQ ID NO: 250 or a Heavy Chain Variable Region (VH) having at least 89%, 90%, 91%, 92%, 93%, 94%, 95%, 96%, 97%, 98% or 99% sequence identity to the amino acid sequence of SEQ ID NO: 250; and a Light Chain Variable Region (VL) comprising the amino acid sequence of SEQ ID NO: 254; or
y. a Heavy Chain Variable Region (VH) comprising the amino acid sequence of SEQ ID NO: 260 or a Heavy Chain Variable Region (VH) having at least 90%, 91%, 92%, 93%, 94%, 95%, 96%, 97%, 98% or 99% sequence identity to the amino acid sequence of SEQ ID NO: 260; and a Light Chain Variable Region (VL) comprising the amino acid sequence of SEQ ID NO: 264; or
z. a Heavy Chain Variable Region (VH) comprising the amino acid sequence of SEQ ID NO: 270 or a Heavy Chain Variable Region (VH) having at least 91%, 92%, 93%, 94%, 95%, 96%, 97%, 98% or 99% sequence identity to the amino acid sequence of SEQ ID NO: 270; and a Light Chain Variable Region (VL) comprising the amino acid sequence of SEQ ID NO: 274; or
aa. a Heavy Chain Variable Region (VH) comprising the amino acid sequence of SEQ ID NO: 280 or a Heavy Chain Variable Region (VH) having at least 87%, 88%, 89%, 90%, 91%, 92%, 93%, 94%, 95%, 96%, 97%, 98% or 99% sequence identity to the amino acid sequence of SEQ ID NO: 280; and a Light Chain Variable Region (VL) comprising the amino acid sequence of SEQ ID NO: 284; or
bb. a Heavy Chain Variable Region (VH) comprising the amino acid sequence of SEQ ID NO: 290 or a Heavy Chain Variable Region (VH) having at least 92%, 93%, 94%, 95%, 96%, 97%, 98% or 99% sequence identity to the amino acid sequence of SEQ ID NO: 290; and a Light Chain Variable Region (VL) comprising the amino acid sequence of SEQ ID NO: 294; or
cc. a Heavy Chain Variable Region (VH) comprising the amino acid sequence of SEQ ID NO: 300 or a Heavy Chain Variable Region (VH) having at least 95%, 96%, 97%, 98% or 99% sequence identity to the amino acid sequence of SEQ ID NO: 300; and a Light Chain Variable Region (VL) comprising the amino acid sequence of SEQ ID NO: 304; or
dd. a Heavy Chain Variable Region (VH) comprising the amino acid sequence of SEQ ID NO: 310 or a Heavy Chain Variable Region (VH) having at least 95%, 96%, 97%, 98% or 99% sequence identity to the amino acid sequence of SEQ ID NO: 310; and a Light Chain Variable Region (VL) comprising the amino acid sequence of SEQ ID NO: 314; or
ee. a Heavy Chain Variable Region (VH) comprising the amino acid sequence of SEQ ID NO: 320 or a Heavy Chain Variable Region (VH) having at least 93%, 94%, 95%, 96%, 97%, 98% or 99% sequence identity to the amino acid sequence of SEQ ID NO: 320; and a Light Chain Variable Region (VL) comprising the amino acid sequence of SEQ ID NO: 324; or
ff. a Heavy Chain Variable Region (VH) comprising the amino acid sequence of SEQ ID NO: 330 or a Heavy Chain Variable Region (VH) having at least 87%, 88%, 89%, 90%, 91%, 92%, 93%, 94%, 95%, 96%, 97%, 98% or 99% sequence identity to the amino acid sequence of SEQ ID NO: 330; and a Light Chain Variable Region (VL) comprising the amino acid sequence of SEQ ID NO: 334; or
gg. a Heavy Chain Variable Region (VH) comprising the amino acid sequence of SEQ ID NO: 340 or a Heavy Chain Variable Region (VH) having at least 89%, 90%, 91%, 92%, 93%, 94%, 95%, 96%, 97%, 98% or 99% sequence identity to the amino acid sequence of SEQ ID NO: 340; and a Light Chain Variable Region (VL) comprising the amino acid sequence of SEQ ID NO: 344; or
hh. a Heavy Chain Variable Region (VH) comprising the amino acid sequence of SEQ ID NO: 350 or a Heavy Chain Variable Region (VH) having at least 89%, 90%, 91%, 92%, 93%, 94%, 95%, 96%, 97%, 98% or 99% sequence identity to the amino acid sequence of SEQ ID NO: 350; and a Light Chain Variable Region (VL) comprising the amino acid sequence of SEQ ID NO: 354; or
ii. a Heavy Chain Variable Region (VH) comprising the amino acid sequence of SEQ ID NO: 360 or a Heavy Chain Variable Region (VH) having at least 92%, 93%, 94%, 95%, 96%, 97%, 98% or 99% sequence identity to the amino acid sequence of SEQ ID NO: 360; and a Light Chain Variable Region (VL) comprising the amino acid sequence of SEQ ID NO: 364; or
jj. a Heavy Chain Variable Region (VH) comprising the amino acid sequence of SEQ ID NO: 370 or a Heavy Chain Variable Region (VH) having at least 85%, 86%, 87%, 88%, 89%, 90%, 91%, 92%, 93%, 94%, 95%, 96%, 97%, 98% or 99% sequence identity to the amino acid sequence of SEQ ID NO: 370; and a Light Chain Variable Region (VL) comprising the amino acid sequence of SEQ ID NO: 374; or
kk. a Heavy Chain Variable Region (VH) comprising the amino acid sequence of SEQ ID NO: 380 or a Heavy Chain Variable Region (VH) having at least 86%, 87%, 88%, 89%, 90%, 91%, 92%, 93%, 94%, 95%, 96%, 97%, 98% or 99% sequence identity to the amino acid sequence of SEQ ID NO: 380; and a Light Chain Variable Region (VL) comprising the amino acid sequence of SEQ ID NO: 384; or
ll. a Heavy Chain Variable Region (VH) comprising the amino acid sequence of SEQ ID NO: 390 or a Heavy Chain Variable Region (VH) having at least 95%, 96%, 97%, 98% or 99% sequence identity to the amino acid sequence of SEQ ID NO: 390; and a Light Chain Variable Region (VL) comprising the amino acid sequence of SEQ ID NO: 394.

In an embodiment of the invention, an ASC binding molecule, in particular an anti-ASC antibody or an antigen-binding fragment thereof of the invention, comprises:
a. a Heavy Chain Variable Region (VH) comprising the amino acid sequence of SEQ ID NO: and a Light Chain Variable Region (VL) comprising the amino acid sequence of SEQ ID NO: 14 or a Light Chain Variable Region (VL) having at least 98% or 99% sequence identity to the amino acid sequence of SEQ ID NO: 14; or
b. a Heavy Chain Variable Region (VH) comprising the amino acid sequence of SEQ ID NO: and a Light Chain Variable Region (VL) comprising the amino acid sequence of SEQ ID NO: 24 or a Light Chain Variable Region (VL) having at least 95%, 96%, 97%, 98% or 99% sequence identity to the amino acid sequence of SEQ ID NO: 24; or
c. a Heavy Chain Variable Region (VH) comprising the amino acid sequence of SEQ ID NO: and a Light Chain Variable Region (VL) comprising the amino acid sequence of SEQ ID NO: 14 or a Light Chain Variable Region (VL) having at least 93%, 94%, 95%, 96%, 97%, 98% or 99% sequence identity to the amino acid sequence of SEQ ID NO: 34; or
d. a Heavy Chain Variable Region (VH) comprising the amino acid sequence of SEQ ID NO: and a Light Chain Variable Region (VL) comprising the amino acid sequence of SEQ ID NO: 44 or a Light Chain Variable Region (VL) having at least 96%, 97%, 98% or 99% sequence identity to the amino acid sequence of SEQ ID NO: 44; or
e. a Heavy Chain Variable Region (VH) comprising the amino acid sequence of SEQ ID NO: 50 and a Light Chain Variable Region (VL) comprising the amino acid sequence of SEQ ID NO: 54 or a Light Chain Variable Region (VL) having at least 96%, 97%, 98% or 99% sequence identity to the amino acid sequence of SEQ ID NO: 54; or
f. a Heavy Chain Variable Region (VH) comprising the amino acid sequence of SEQ ID NO: 60 and a Light Chain Variable Region (VL) comprising the amino acid sequence of SEQ ID NO: 64; or
g. a Heavy Chain Variable Region (VH) comprising the amino acid sequence of SEQ ID NO: 70 and a Light Chain Variable Region (VL) comprising the amino acid sequence of SEQ ID NO: 74 or a Light Chain Variable Region (VL) having at least 97%, 98% or 99% sequence identity to the amino acid sequence of SEQ ID NO: 74; or
h. a Heavy Chain Variable Region (VH) comprising the amino acid sequence of SEQ ID NO: 80 and a Light Chain Variable Region (VL) comprising the amino acid sequence of SEQ ID NO: 84 or a Light Chain Variable Region (VL) having at least 95%, 96%, 97%, 98% or 99% sequence identity to the amino acid sequence of SEQ ID NO: 84; or
i. a Heavy Chain Variable Region (VH) comprising the amino acid sequence of SEQ ID NO: 90 and a Light Chain Variable Region (VL) comprising the amino acid sequence of SEQ ID NO: 94 or a Light Chain Variable Region (VL) having at least 93%, 94%, 95%, 96%, 97%, 98% or 99% sequence identity to the amino acid sequence of SEQ ID NO: 94; or
j. a Heavy Chain Variable Region (VH) comprising the amino acid sequence of SEQ ID NO: 110 and a Light Chain Variable Region (VL) comprising the amino acid sequence of SEQ ID NO: 114 or a Light Chain Variable Region (VL) having at least 98% or 99% sequence identity to the amino acid sequence of SEQ ID NO: 114; or
k. a Heavy Chain Variable Region (VH) comprising the amino acid sequence of SEQ ID NO: 120 and a Light Chain Variable Region (VL) comprising the amino acid sequence of SEQ ID NO: 124 or a Light Chain Variable Region (VL) having at least 98% or 99% sequence identity to the amino acid sequence of SEQ ID NO: 124; or
l. a Heavy Chain Variable Region (VH) comprising the amino acid sequence of SEQ ID NO: 130 and a Light Chain Variable Region (VL) comprising the amino acid sequence of SEQ ID NO: 134 or a Light Chain Variable Region (VL) having at least 98% or 99% sequence identity to the amino acid sequence of SEQ ID NO: 134; or
m. a Heavy Chain Variable Region (VH) comprising the amino acid sequence of SEQ ID NO: 140 or a Light Chain Variable Region (VL) having at least 98% or 99% sequence identity to the amino acid sequence of SEQ ID NO: 144; or
n. a Heavy Chain Variable Region (VH) comprising the amino acid sequence of SEQ ID NO: 150 and a Light Chain Variable Region (VL) comprising the amino acid sequence of SEQ ID NO: 154 or a Light Chain Variable Region (VL) having at least 93%, 94%, 95%, 96%, 97%, 98% or 99% sequence identity to the amino acid sequence of SEQ ID NO: 154; or
o. a Heavy Chain Variable Region (VH) comprising the amino acid sequence of SEQ ID NO: 160 and a Light Chain Variable Region (VL) comprising the amino acid sequence of SEQ ID NO: 164 or a Light Chain Variable Region (VL) having at least 96%, 97%, 98% or 99% sequence identity to the amino acid sequence of SEQ ID NO: 164; or
p. a Heavy Chain Variable Region (VH) comprising the amino acid sequence of SEQ ID NO: 170 and a Light Chain Variable Region (VL) comprising the amino acid sequence of SEQ ID NO: 174 or a Light Chain Variable Region (VL) having at least 93%, 94%, 95%, 96%, 97%, 98% or 99% sequence identity to the amino acid sequence of SEQ ID NO: 174; or
q. a Heavy Chain Variable Region (VH) comprising the amino acid sequence of SEQ ID NO: 180 and a Light Chain Variable Region (VL) comprising the amino acid sequence of SEQ ID NO: 184 or a Light Chain Variable Region (VL) having at least 97%, 98% or 99% sequence identity to the amino acid sequence of SEQ ID NO: 184; or
r. a Heavy Chain Variable Region (VH) comprising the amino acid sequence of SEQ ID NO: 190 and a Light Chain Variable Region (VL) comprising the amino acid sequence of SEQ ID NO: 194; or
s. a Heavy Chain Variable Region (VH) comprising the amino acid sequence of SEQ ID NO: 200 and a Light Chain Variable Region (VL) comprising the amino acid sequence of SEQ ID NO: 204 or a Light Chain Variable Region (VL) having at least 99% sequence identity to the amino acid sequence of SEQ ID NO: 204; or
t. a Heavy Chain Variable Region (VH) comprising the amino acid sequence of SEQ ID NO: 210 and a Light Chain Variable Region (VL) comprising the amino acid sequence of SEQ ID NO: 214 or a Light Chain Variable Region (VL) having at least 96%, 97%, 98% or 99% sequence identity to the amino acid sequence of SEQ ID NO: 214; or
u. a Heavy Chain Variable Region (VH) comprising the amino acid sequence of SEQ ID NO: 220 and a Light Chain Variable Region (VL) comprising the amino acid sequence of SEQ ID NO: 224 or a Light Chain Variable Region (VL) having at least 93%, 94%, 95%, 96%, 97%, 98% or 99% sequence identity to the amino acid sequence of SEQ ID NO: 224; or
v. a Heavy Chain Variable Region (VH) comprising the amino acid sequence of SEQ ID NO: 230 and a Light Chain Variable Region (VL) comprising the amino acid sequence of SEQ ID NO: 234 or a Light Chain Variable Region (VL) having at least 92%, 93%, 94%, 95%, 96%, 97%, 98% or 99% sequence identity to the amino acid sequence of SEQ ID NO: 234; or
w. a Heavy Chain Variable Region (VH) comprising the amino acid sequence of SEQ ID NO: 240 and a Light Chain Variable Region (VL) comprising the amino acid sequence of SEQ ID NO: 244 or a Light Chain Variable Region (VL) having at least 97%, 98% or 99% sequence identity to the amino acid sequence of SEQ ID NO: 244; or
x. a Heavy Chain Variable Region (VH) comprising the amino acid sequence of SEQ ID NO: 250 and a Light Chain Variable Region (VL) comprising the amino acid sequence of SEQ ID NO: 254 or a Light Chain Variable Region (VL) having at least 97%, 98% or 99% sequence identity to the amino acid sequence of SEQ ID NO: 254; or
y. a Heavy Chain Variable Region (VH) comprising the amino acid sequence of SEQ ID NO: 260 and a Light Chain Variable Region (VL) comprising the amino acid sequence of SEQ ID NO: 264 or a Light Chain Variable Region (VL) having at least 97%, 98% or 99% sequence identity to the amino acid sequence of SEQ ID NO: 264; or
z. a Heavy Chain Variable Region (VH) comprising the amino acid sequence of SEQ ID NO: 270 and a Light Chain Variable Region (VL) comprising the amino acid sequence of SEQ ID NO: 274 or a Light Chain Variable Region (VL) having at least or 99% sequence identity to the amino acid sequence of SEQ ID NO: 274; or
aa. a Heavy Chain Variable Region (VH) comprising the amino acid sequence of SEQ ID NO: 280 and a Light Chain Variable Region (VL) comprising the amino acid sequence of SEQ ID NO: 284 or a Light Chain Variable Region (VL) having at least 98% or 99% sequence identity to the amino acid sequence of SEQ ID NO: 284; or
bb. a Heavy Chain Variable Region (VH) comprising the amino acid sequence of SEQ ID NO: 290 and a Light Chain Variable Region (VL) comprising the amino acid sequence of SEQ ID NO: 294 or a Light Chain Variable Region (VL) having at least 99% sequence identity to the amino acid sequence of SEQ ID NO: 294; or
cc. a Heavy Chain Variable Region (VH) comprising the amino acid sequence of SEQ ID NO: 300 and a Light Chain Variable Region (VL) comprising the amino acid sequence of SEQ ID NO: 304 or a Light Chain Variable Region (VL) having at least 98% or 99% sequence identity to the amino acid sequence of SEQ ID NO: 304; or
dd. a Heavy Chain Variable Region (VH) comprising the amino acid sequence of SEQ ID NO: 310 and a Light Chain Variable Region (VL) comprising the amino acid sequence of SEQ ID NO: 314 or a Light Chain Variable Region (VL) having at least 97%, 98% or 99% sequence identity to the amino acid sequence of SEQ ID NO: 314; or
ee. a Heavy Chain Variable Region (VH) comprising the amino acid sequence of SEQ ID NO: 320 and a Light Chain Variable Region (VL) comprising the amino acid sequence of SEQ ID NO: 324 or a Light Chain Variable Region (VL) having at least 94%, 95%, 96%, 97%, 98% or 99% sequence identity to the amino acid sequence of SEQ ID NO: 324; or
ff. a Heavy Chain Variable Region (VH) comprising the amino acid sequence of SEQ ID NO: 330 and a Light Chain Variable Region (VL) comprising the amino acid sequence of SEQ ID NO: 334 or a Light Chain Variable Region (VL) having at least 96%, 97%, 98% or 99% sequence identity to the amino acid sequence of SEQ ID NO: 334; or
gg. a Heavy Chain Variable Region (VH) comprising the amino acid sequence of SEQ ID NO: 340 and a Light Chain Variable Region (VL) comprising the amino acid sequence of SEQ ID NO: 344 or a Light Chain Variable Region (VL) having at least 89%, 90%, 91%, 92%, 93%, 94%, 95%, 96%, 97%, 98% or 99% sequence identity to the amino acid sequence of SEQ ID NO: 344; or
hh. a Heavy Chain Variable Region (VH) comprising the amino acid sequence of SEQ ID NO: 350 and a Light Chain Variable Region (VL) comprising the amino acid sequence of SEQ ID NO: 354 or a Light Chain Variable Region (VL) having at least 96%, 97%, 98% or 99% sequence identity to the amino acid sequence of SEQ ID NO: 354; or
ii. a Heavy Chain Variable Region (VH) comprising the amino acid sequence of SEQ ID NO: 360 and a Light Chain Variable Region (VL) comprising the amino acid sequence of SEQ ID NO: 364 or a Light Chain Variable Region (VL) having at least 98% or 99% sequence identity to the amino acid sequence of SEQ ID NO: 364; or
jj. a Heavy Chain Variable Region (VH) comprising the amino acid sequence of SEQ ID NO: 370 and a Light Chain Variable Region (VL) comprising the amino acid sequence of SEQ ID NO: 374 or a Light Chain Variable Region (VL) having at least 94%, 95%, 96%, 97%, 98% or 99% sequence identity to the amino acid sequence of SEQ ID NO: 374; or
kk. a Heavy Chain Variable Region (VH) comprising the amino acid sequence of SEQ ID NO: 380 and a Light Chain Variable Region (VL) comprising the amino acid sequence of SEQ ID NO: 384 or a Light Chain Variable Region (VL) having at least 93%, 94%, 95%, 96%, 97%, 98% or 99% sequence identity to the amino acid sequence of SEQ ID NO: 384; or
ll. a Heavy Chain Variable Region (VH) comprising the amino acid sequence of SEQ ID NO: 390 and a Light Chain Variable Region (VL) comprising the amino acid sequence of SEQ ID NO: 394.

In one embodiment, an ASC binding molecule, particularly an anti-ASC antibody or an antigen-binding fragment thereof of the invention comprises a Heavy Chain Variable Region comprising a VH-CDR1 comprising the amino acid sequence of SEQ ID NO: 201, a VH-CDR2 comprising the amino acid sequence of SEQ ID NO: 202, and a VH-CDR3 comprising the amino acid sequence 203.

In one embodiment, an ASC binding molecule, particularly an anti-ASC antibody or an antigen-binding fragment thereof of the invention may comprise a Light Chain Variable Region (VL) which comprises a VL-CDR1 comprising the amino sequence SEQ ID NO: 205, a VL-CDR2 comprising the amino acid sequence of SEQ ID NO: 206, and a VL-CDR3 comprising the amino acid sequence of SEQ ID NO: 207.

In one embodiment, an ASC binding molecule, particularly an anti-ASC antibody or an antigen-binding fragment thereof of the invention comprises a VH-CDR1 comprising the amino acid sequence of SEQ ID NO: 201, a VH-CDR2 comprising the amino acid sequence of SEQ ID NO: 202, a VH-CDR3 comprising the amino acid sequence 203, a VL-CDR1 comprising the amino sequence SEQ ID NO: 205, a VL-CDR2 comprising the amino acid sequence of SEQ ID NO: 206, and a VL-CDR3 comprising the amino acid sequence of SEQ ID NO: 207.

In an embodiment of the invention, an ASC binding molecule, in particular an anti-ASC antibody or an antigen-binding fragment thereof, comprises a Heavy Chain Variable Region (VH) may comprise the amino acid sequence of SEQ ID NO: 200 or a Heavy Chain Variable Region (VH) having at least 92%, 93%, 94%, 95%, 96%, 97%, 98% or 99% sequence identity to the amino acid sequence of SEQ ID NO: 200; and a Light Chain Variable Region (VL) comprising the amino acid sequence of SEQ ID NO: 204.

In an embodiment of the invention, an ASC binding molecule, in particular an anti-ASC antibody or an antigen-binding fragment thereof of the invention, may comprise a Heavy Chain Variable Region (VH) comprising the amino acid sequence of SEQ ID NO: 200 and a Light Chain Variable Region (VL) comprising the amino acid sequence of SEQ ID NO: 204 or a Light Chain Variable Region (VL) having at least 99% sequence identity to the amino acid sequence of SEQ ID NO: 204; or In an embodiment of the invention, an ASC binding molecule, in particular an anti-ASC antibody or an antigen-binding fragment thereof of the invention, may comprise a Heavy Chain Variable Region (VH) comprising the amino acid sequence of SEQ ID NO: 200 or a Heavy Chain Variable Region (VH) having at least 92%, 93%, 94%, 95%, 96%, 97%, 98% or 99% sequence identity to the amino acid sequence of SEQ ID NO: 200; and a Light Chain Variable Region (VL) comprising the amino acid sequence of SEQ ID NO: 204 or a Light Chain Variable Region (VL) having at least 98% or 99% sequence identity to the amino acid sequence of SEQ ID NO: 204.

In certain embodiments, the ASC binding molecule of the invention is a monoclonal antibody or an antigen-binding fragment thereof. In certain embodiments, the anti-ASC antibody or an antigen-binding fragment thereof of the invention is an IgA, IgD, IgE, IgM, IgG1, IgG2, IgG2a, IgG2b, IgG3 or IgG4 antibody or antigen-binding fragment thereof. The anti-ASC antibody or an antigen-binding fragment thereof may be human or mouse, preferably human IgA, IgD, IgE, IgM, IgG1, IgG2, IgG2a, IgG2b, IgG3 or IgG4. In one embodiment, the ASC binding molecule, particularly anti-ASC antibody or an antigen-binding fragment thereof is a human IgG4 isotype including the S228P mutation.

In certain embodiments, an antibody provided herein is selected from ACI-8016-416E6G4-AB1, ACI-8016-402H11C9-Ab1, ACI-8016-203B12C3-AB1, ACI-8016-421B10C12D2-AB1, ACI-8016-417E12A8-AB1, ACI-8016-413G10A5-AB1, ACI-8016-407E10A9-AB1, ACI-8016-203G8B10-AB1, ACI-8016-401H9B7-AB1, ACI-8016-1112B3D7-AB1, ACI-8018-2221B7F1-AB1, ACI-8019-2314F6H11-AB1, ACI-8016-207E8B2-AB1, ACI-8016-2A1B12-AB1, ACI-8016-17H1G2-AB1, ACI-8016-18F4C12-AB1, ACI-8016-23E5F7-AB1, ACI-8016-23E5F7-AB2, ACI-8016-26A1G2-AB1, ACI-8016-32B6C7-AB1, ACI-8016-22D3A6-AB1, ACI-8016-31F10C5-AB1, ACI-8016-19E6D4-AB1, ACI-8016-3E6B11-AB1, ACI-8016-11A3F3-AB1, ACI-8016-14G5B8-AB1, ACI-8016-22A10F8-AB1, ACI-8016-27A1G4-AB1, ACI-8016-29C5E11-AB1, ACI-8016-7G3B5-AB1, ACI-8016-2504F3D9-AB1, ACI-8016-2516A8C6-AB1, ACI-8016-2602H6F10-AB1, ACI-8016-2609F4A9-AB1, ACI-8016-2610H7D3-AB1, ACI-8016-2614C3B2-AB1, ACI-8016-2617C3A8-AB1, ACI-8016-2622E12F11-AB1, ACI-8016-2626B9D3-AB1, or ACI-8016-2629E8D1-AB1 as set forth in Table 17. In one embodiment, the antibody provide herein may be selected from Table 17.

In certain embodiments, an antibody provided herein is selected from ACI-8016-32B6C7-AB1 and ACI-8016-18F4C12-AB1.

In certain preferred embodiments, an antibody provided herein is ACI-8016-32B6C7-AB1.

In one embodiment the ASC binding molecule may comprise:
a) a VH-CDR1 comprising the amino acid sequence of SEQ ID NO: 201, a VH-CDR2 comprising the amino acid sequence of SEQ ID NO: 202; and a VH-CDR3 comprising the amino acid sequence SEQ ID NO: 203, a VL-CDR1 comprising the amino sequence SEQ ID NO: 205; a VL-CDR2 comprising the amino sequence SEQ ID NO: 206, and a VL-CDR3 comprising the amino sequence SEQ ID NO: 207; or
b) a VH-CDR1 comprising the amino acid sequence of SEQ ID NO: 201, a VH-CDR2 comprising the amino acid sequence of SEQ ID NO: 412 and a VH-CDR3 comprising the amino acid sequence SEQ ID NO: 203; a VL-CDR1 comprising the amino sequence SEQ ID NO: 205; a VL-CDR2 comprising the amino sequence SEQ ID NO: 206, and a VL-CDR3 comprising the amino sequence SEQ ID NO: 207; or
c) a VH-CDR1 comprising the amino acid sequence of SEQ ID NO: 201, a VH-CDR2 comprising the amino acid sequence of SEQ ID NO: 412 and a VH-CDR3 comprising the amino acid sequence SEQ ID NO: 203; a VL-CDR1 comprising the amino sequence SEQ ID NO: 435; a VL-CDR2 comprising the amino sequence SEQ ID NO: 206, and a VL-CDR3 comprising the amino sequence SEQ ID NO: 207; or
d) a VH-CDR1 comprising the amino acid sequence of SEQ ID NO: 201, a VH-CDR2 comprising the amino acid sequence of SEQ ID NO: 462 and a VH-CDR3 comprising the amino acid sequence SEQ ID NO: 203; a VL-CDR1 comprising the amino sequence SEQ ID NO: 205; a VL-CDR2 comprising the amino sequence SEQ ID NO: 206, and a VL-CDR3 comprising the amino sequence SEQ ID NO: 207; or
e) a VH-CDR1 comprising the amino acid sequence of SEQ ID NO: 201, a VH-CDR2 comprising the amino acid sequence of SEQ ID NO: 462 and a VH-CDR3 comprising the amino acid sequence SEQ ID NO: 203; a VL-CDR1 comprising the amino sequence SEQ ID NO: 435; a VL-CDR2 comprising the amino sequence SEQ ID NO: 206, and a VL-CDR3 comprising the amino sequence SEQ ID NO: 207; or
f) a VH-CDR1 comprising the amino acid sequence of SEQ ID NO: 201, a VH-CDR2 comprising the amino acid sequence of SEQ ID NO: 472 and a VH-CDR3 comprising the amino acid sequence SEQ ID NO: 203; a VL-CDR1 comprising the amino sequence SEQ ID NO: 205; a VL-CDR2 comprising the amino sequence SEQ ID NO: 206, and a VL-CDR3 comprising the amino sequence SEQ ID NO: 207; or
g) a VH-CDR1 comprising the amino acid sequence of SEQ ID NO: 201, a VH-CDR2 comprising the amino acid sequence of SEQ ID NO: 472 and a VH-CDR3 comprising the amino acid sequence SEQ ID NO: 203; a VL-CDR1 comprising the amino sequence SEQ ID NO: 435; a VL-CDR2 comprising the amino sequence SEQ ID NO: 206, and a VL-CDR3 comprising the amino sequence SEQ ID NO: 207.

In one embodiment the ASC binding molecule may comprise:
a) a VH-CDR1 comprising the amino acid sequence of SEQ ID NO: 201, a VH-CDR2 comprising the amino acid sequence of SEQ ID NO: 202; and a VH-CDR3 comprising the amino acid sequence SEQ ID NO: 203; or
b) a VH-CDR1 comprising the amino acid sequence of SEQ ID NO: 201, a VH-CDR2 comprising the amino acid sequence of SEQ ID NO: 412 and a VH-CDR3 comprising the amino acid sequence SEQ ID NO: 203; or
c) a VH-CDR1 comprising the amino acid sequence of SEQ ID NO: 201, a VH-CDR2 comprising the amino acid sequence of SEQ ID NO: 462 and a VH-CDR3 comprising the amino acid sequence SEQ ID NO: 203; or
d) a VH-CDR1 comprising the amino acid sequence of SEQ ID NO: 201, a VH-CDR2 comprising the amino acid sequence of SEQ ID NO: 472 and a VH-CDR3 comprising the amino acid sequence SEQ ID NO: 203.

In one embodiment, the ASC binding molecule may comprise:
a) a VL-CDR1 comprising the amino acid sequence of SEQ ID NO: 205, a VL-CDR2 comprising the amino acid sequence of SEQ ID NO: 206, a VL-CDR3 comprising the amino acid sequence SEQ ID NO: 207; or
b) a VL-CDR1 comprising the amino acid sequence of SEQ ID NO: 435, a VL-CDR2 comprising the amino acid sequence of SEQ ID NO: 206, a VL-CDR3 comprising the amino acid sequence SEQ ID NO: 207.

In one embodiment the ASC binding molecule may comprise:
a) a Heavy Chain Variable Region (VH) comprising the amino acid sequence of SEQ ID NO: 400, or having at least 93%, 94%, 95%, 96%, 97%, 98% or 99% sequence identity to the amino acid sequence of SEQ ID NO: 400 and a Light Chain Variable Region (VL) comprising the amino acid sequence of SEQ ID NO: 404 or a Light Chain Variable Region (VL) having at least 95%, 96%, 97%, 98% or 99% sequence identity to the amino acid sequence of SEQ ID NO: 404; or
b) a Heavy Chain Variable Region (VH) comprising the amino acid sequence of SEQ ID NO: 410, or having at least 93%, 94%, 95%, 96%, 97%, 98% or 99% sequence identity to the amino acid sequence of SEQ ID NO: 410 and a Light Chain Variable Region (VL) comprising the amino acid sequence of SEQ ID NO: 414 or a Light Chain Variable Region (VL) having at least 95%, 96%, 97%, 98% or 99% sequence identity to the amino acid sequence of SEQ ID NO: 414; or
c) a Heavy Chain Variable Region (VH) comprising the amino acid sequence of SEQ ID NO: 420, or having at least 93%, 94%, 95%, 96%, 97%, 98% or 99% sequence identity to the amino acid sequence of SEQ ID NO: 420 and a Light Chain Variable Region (VL) comprising the amino acid sequence of SEQ ID NO: 424 or a Light Chain Variable Region (VL) having at least 95%, 96%, 97%, 98% or 99% sequence identity to the amino acid sequence of SEQ ID NO: 424; or
d) a Heavy Chain Variable Region (VH) comprising the amino acid sequence of SEQ ID NO: 430, or having at least 93%, 94%, 95%, 96%, 97%, 98% or 99% sequence identity to the amino acid sequence of SEQ ID NO: 430 and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
e) a Heavy Chain Variable Region (VH) comprising the amino acid sequence of SEQ ID NO: 440, or having at least 93%, 94%, 95%, 96%, 97%, 98% or 99% sequence identity to the amino acid sequence of SEQ ID NO: 440 and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
f) a Heavy Chain Variable Region (VH) comprising the amino acid sequence of SEQ ID NO: 450, or having at least 93%, 94%, 95%, 96%, 97%, 98% or 99% sequence identity to the amino acid sequence of SEQ ID NO: 450 and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
g) a Heavy Chain Variable Region (VH) comprising the amino acid sequence of SEQ ID NO: 460, or having at least 93%, 94%, 95%, 96%, 97%, 98% or 99% sequence identity to the amino acid sequence of SEQ ID NO: 460 and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
h) a Heavy Chain Variable Region (VH) comprising the amino acid sequence of SEQ ID NO: 470, or having at least 93%, 94%, 95%, 96%, 97%, 98% or 99% sequence identity to the amino acid sequence of SEQ ID NO: 470 and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
i) a Heavy Chain Variable Region (VH) comprising the amino acid sequence of SEQ ID NO: 480, or having at least 93%, 94%, 95%, 96%, 97%, 98% or 99% sequence identity to the amino acid sequence of SEQ ID NO: 480 and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
j) a Heavy Chain Variable Region (VH) comprising the amino acid sequence of SEQ ID NO: 490, or having at least 93%, 94%, 95%, 96%, 97%, 98% or 99% sequence identity to the amino acid sequence of SEQ ID NO: 490 and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
k) a Heavy Chain Variable Region (VH) comprising the amino acid sequence of SEQ ID NO: 500, or having at least 93%, 94%, 95%, 96%, 97%, 98% or 99% sequence identity to the amino acid sequence of SEQ ID NO: 500 and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
l) a Heavy Chain Variable Region (VH) comprising the amino acid sequence of SEQ ID NO: 510, or having at least 93%, 94%, 95%, 96%, 97%, 98% or 99% sequence identity to the amino acid sequence of SEQ ID NO: 510 and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
m) a Heavy Chain Variable Region (VH) comprising the amino acid sequence of SEQ ID NO: 520, or having at least 93%, 94%, 95%, 96%, 97%, 98% or 99% sequence identity to the amino acid sequence of SEQ ID NO: 520 and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
n) a Heavy Chain Variable Region (VH) comprising the amino acid sequence of SEQ ID NO: 530, or having at least 93%, 94%, 95%, 96%, 97%, 98% or 99% sequence identity to the amino acid sequence of SEQ ID NO: 530 and a Light Chain Variable Region (VL) comprising the amino acid sequence of SEQ ID NO: 424 or a Light Chain Variable Region (VL) having at least 95%, 96%, 97%, 98% or 99% sequence identity to the amino acid sequence of SEQ ID NO: 424; or
o) a Heavy Chain Variable Region (VH) comprising the amino acid sequence of SEQ ID NO: 540, or having at least 93%, 94%, 95%, 96%, 97%, 98% or 99% sequence identity to the amino acid sequence of SEQ ID NO: 540 and a Light Chain Variable Region (VL) comprising the amino acid sequence of SEQ ID NO: 424 or a Light Chain Variable Region (VL) having at least 95%, 96%, 97%, 98% or 99% sequence identity to the amino acid sequence of SEQ ID NO: 424; or
p) a Heavy Chain Variable Region (VH) comprising the amino acid sequence of SEQ ID NO: 400, or having at least 93%, 94%, 95%, 96%, 97%, 98% or 99% sequence identity to the amino acid sequence of SEQ ID NO: 400 and a Light Chain Variable Region (VL) comprising the amino acid sequence of SEQ ID NO: 414 or a Light Chain Variable Region (VL) having at least 95%, 96%, 97%, 98% or 99% sequence identity to the amino acid sequence of SEQ ID NO: 414; or
q) a Heavy Chain Variable Region (VH) comprising the amino acid sequence of SEQ ID NO: 410, or having at least 93%, 94%, 95%, 96%, 97%, 98% or 99% sequence identity to the amino acid sequence of SEQ ID NO: 410 and a Light Chain Variable Region (VL) comprising the amino acid sequence of SEQ ID NO: 404 or a Light Chain Variable Region (VL) having at least 95%, 96%, 97%, 98% or 99% sequence identity to the amino acid sequence of SEQ ID NO: 404; or
r) a Heavy Chain Variable Region (VH) comprising the amino acid sequence of SEQ ID NO: 410, or having at least 93%, 94%, 95%, 96%, 97%, 98% or 99% sequence identity to the amino acid sequence of SEQ ID NO: 410 and a Light Chain Variable Region (VL) comprising the amino acid sequence of SEQ ID NO: 424 or a Light Chain Variable Region (VL) having at least 95%, 96%, 97%, 98% or 99% sequence identity to the amino acid sequence of SEQ ID NO: 424; or
s) a Heavy Chain Variable Region (VH) comprising the amino acid sequence of SEQ ID NO: 410, or having at least 93%, 94%, 95%, 96%, 97%, 98% or 99% sequence identity to the amino acid sequence of SEQ ID NO: 410 and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
t) a Heavy Chain Variable Region (VH) comprising the amino acid sequence of SEQ ID NO: 420, or having at least 93%, 94%, 95%, 96%, 97%, 98% or 99% sequence identity to the amino acid sequence of SEQ ID NO: 420 and a Light Chain Variable Region (VL) comprising the amino acid sequence of SEQ ID NO: 404 or a Light Chain Variable Region (VL) having at least 95%, 96%, 97%, 98% or 99% sequence identity to the amino acid sequence of SEQ ID NO: 404; or
u) a Heavy Chain Variable Region (VH) comprising the amino acid sequence of SEQ ID NO: 420, or having at least 93%, 94%, 95%, 96%, 97%, 98% or 99% sequence identity to the amino acid sequence of SEQ ID NO: 420 and a Light Chain Variable Region (VL) comprising the amino acid sequence of SEQ ID NO: 414 or a Light Chain Variable Region (VL) having at least 95%, 96%, 97%, 98% or 99% sequence identity to the amino acid sequence of SEQ ID NO: 414; or
v) a Heavy Chain Variable Region (VH) comprising the amino acid sequence of SEQ ID NO: 430, or having at least 93%, 94%, 95%, 96%, 97%, 98% or 99% sequence identity to the amino acid sequence of SEQ ID NO: 430 and a Light Chain Variable Region (VL) comprising the amino acid sequence of SEQ ID NO: 404 or a Light Chain Variable Region (VL) having at least 95%, 96%, 97%, 98% or 99% sequence identity to the amino acid sequence of SEQ ID NO: 404; or
w) a Heavy Chain Variable Region (VH) comprising the amino acid sequence of SEQ ID NO: 430, or having at least 93%, 94%, 95%, 96%, 97%, 98% or 99% sequence identity to the amino acid sequence of SEQ ID NO: 430 and a Light Chain Variable Region (VL) comprising the amino acid sequence of SEQ ID NO: 414 or a Light Chain Variable Region (VL) having at least 95%, 96%, 97%, 98% or 99% sequence identity to the amino acid sequence of SEQ ID NO: 414; or
x) a Heavy Chain Variable Region (VH) comprising the amino acid sequence of SEQ ID NO: 400, or having at least 93%, 94%, 95%, 96%, 97%, 98% or 99% sequence identity to the amino acid sequence of SEQ ID NO: 400 and a Light Chain Variable Region (VL) comprising the amino acid sequence of SEQ ID NO: 424 or a Light Chain Variable Region (VL) having at least 95%, 96%, 97%, 98% or 99% sequence identity to the amino acid sequence of SEQ ID NO: 424; or
y) a Heavy Chain Variable Region (VH) comprising the amino acid sequence of SEQ ID NO: 450, or having at least 93%, 94%, 95%, 96%, 97%, 98% or 99% sequence identity to the amino acid sequence of SEQ ID NO: 450 and a Light Chain Variable Region (VL) comprising the amino acid sequence of SEQ ID NO: 424 or a Light Chain Variable Region (VL) having at least 95%, 96%, 97%, 98% or 99% sequence identity to the amino acid sequence of SEQ ID NO: 424; or
z) a Heavy Chain Variable Region (VH) comprising the amino acid sequence of SEQ ID NO: 480, or having at least 93%, 94%, 95%, 96%, 97%, 98% or 99% sequence identity to the amino acid sequence of SEQ ID NO: 480 and a Light Chain Variable Region (VL) comprising the amino acid sequence of SEQ ID NO: 424 or a Light Chain Variable Region (VL) having at least 95%, 96%, 97%, 98% or 99% sequence identity to the amino acid sequence of SEQ ID NO: 424; or
aa) a Heavy Chain Variable Region (VH) comprising the amino acid sequence of SEQ ID NO: 520, or having at least 93%, 94%, 95%, 96%, 97%, 98% or 99% sequence identity to the amino acid sequence of SEQ ID NO: 520 and a Light Chain Variable Region (VL) comprising the amino acid sequence of SEQ ID NO: 424 or a Light Chain Variable Region (VL) having at least 95%, 96%, 97%, 98% or 99% sequence identity to the amino acid sequence of SEQ ID NO: 424; or
bb) a Heavy Chain Variable Region (VH) comprising the amino acid sequence of SEQ ID NO: 430, or having at least 93%, 94%, 95%, 96%, 97%, 98% or 99% sequence identity to the amino acid sequence of SEQ ID NO: 430, and a Light Chain Variable Region (VL) comprising the amino acid sequence of SEQ ID NO: 424 or a Light Chain Variable Region (VL) having at least 95%, 96%, 97%, 98% or 99% sequence identity to the amino acid sequence of SEQ ID NO: 424.

In one embodiment the ASC binding molecule may comprise:
a) a Heavy Chain Variable Region (VH) comprising the amino acid sequence of SEQ ID NO: 400, or having at least 93%, 94%, 95%, 96%, 97%, 98% or 99% sequence identity to the amino acid sequence of SEQ ID NO: 400 and a Light Chain Variable Region (VL) comprising the amino acid sequence of SEQ ID NO: 404; or
b) a Heavy Chain Variable Region (VH) comprising the amino acid sequence of SEQ ID NO: 410, or having at least 93%, 94%, 95%, 96%, 97%, 98% or 99% sequence identity to the amino acid sequence of SEQ ID NO: 410 and a Light Chain Variable Region (VL) comprising the amino acid sequence of SEQ ID NO: 414; or
c) a Heavy Chain Variable Region (VH) comprising the amino acid sequence of SEQ ID NO: 420, or having at least 93%, 94%, 95%, 96%, 97%, 98% or 99% sequence identity to the amino acid sequence of SEQ ID NO: 420 and a Light Chain Variable Region (VL) comprising the amino acid sequence of SEQ ID NO: 424; or
d) a Heavy Chain Variable Region (VH) comprising the amino acid sequence of SEQ ID NO: 430, or having at least 93%, 94%, 95%, 96%, 97%, 98% or 99% sequence identity to the amino acid sequence of SEQ ID NO: 430 and a Light Chain Variable Region (VL) comprising the amino acid sequence of SEQ ID NO: 434; or
e) a Heavy Chain Variable Region (VH) comprising the amino acid sequence of SEQ ID NO: 440, or having at least 93%, 94%, 95%, 96%, 97%, 98% or 99% sequence identity to the amino acid sequence of SEQ ID NO: 440 and a Light Chain Variable Region (VL) comprising the amino acid sequence of SEQ ID NO: 434; or
f) a Heavy Chain Variable Region (VH) comprising the amino acid sequence of SEQ ID NO: 450, or having at least 93%, 94%, 95%, 96%, 97%, 98% or 99% sequence identity to the amino acid sequence of SEQ ID NO: 450 and a Light Chain Variable Region (VL) comprising the amino acid sequence of SEQ ID NO: 434; or
g) a Heavy Chain Variable Region (VH) comprising the amino acid sequence of SEQ ID NO: 460, or having at least 93%, 94%, 95%, 96%, 97%, 98% or 99% sequence identity to the amino acid sequence of SEQ ID NO: 460 and a Light Chain Variable Region (VL) comprising the amino acid sequence of SEQ ID NO: 434; or
h) a Heavy Chain Variable Region (VH) comprising the amino acid sequence of SEQ ID NO: 470, or having at least 93%, 94%, 95%, 96%, 97%, 98% or 99% sequence identity to the amino acid sequence of SEQ ID NO: 470 and a Light Chain Variable Region (VL) comprising the amino acid sequence of SEQ ID NO: 434; or
i) a Heavy Chain Variable Region (VH) comprising the amino acid sequence of SEQ ID NO: 480, or having at least 93%, 94%, 95%, 96%, 97%, 98% or 99% sequence identity to the amino acid sequence of SEQ ID NO: 480 and a Light Chain Variable Region (VL) comprising the amino acid sequence of SEQ ID NO: 434; or
j) a Heavy Chain Variable Region (VH) comprising the amino acid sequence of SEQ ID NO: 490, or having at least 93%, 94%, 95%, 96%, 97%, 98% or 99% sequence identity to the amino acid sequence of SEQ ID NO: 490 and a Light Chain Variable Region (VL) comprising the amino acid sequence of SEQ ID NO: 434; or
k) a Heavy Chain Variable Region (VH) comprising the amino acid sequence of SEQ ID NO: 500, or having at least 93%, 94%, 95%, 96%, 97%, 98% or 99% sequence identity to the amino acid sequence of SEQ ID NO: 500 and a Light Chain Variable Region (VL) comprising the amino acid sequence of SEQ ID NO: 434; or
l) a Heavy Chain Variable Region (VH) comprising the amino acid sequence of SEQ ID NO: 510, or having at least 93%, 94%, 95%, 96%, 97%, 98% or 99% sequence identity to the amino acid sequence of SEQ ID NO: 510 and a Light Chain Variable Region (VL) comprising the amino acid sequence of SEQ ID NO: 434; or
m) a Heavy Chain Variable Region (VH) comprising the amino acid sequence of SEQ ID NO: 520, or having at least 93%, 94%, 95%, 96%, 97%, 98% or 99% sequence identity to the amino acid sequence of SEQ ID NO: 520 and a Light Chain Variable Region (VL) comprising the amino acid sequence of SEQ ID NO: 434; or
n) a Heavy Chain Variable Region (VH) comprising the amino acid sequence of SEQ ID NO: 530, or having at least 93%, 94%, 95%, 96%, 97%, 98% or 99% sequence identity to the amino acid sequence of SEQ ID NO: 530 and a Light Chain Variable Region (VL) comprising the amino acid sequence of SEQ ID NO: 424; or
o) a Heavy Chain Variable Region (VH) comprising the amino acid sequence of SEQ ID NO: 540, or having at least 93%, 94%, 95%, 96%, 97%, 98% or 99% sequence identity to the amino acid sequence of SEQ ID NO: 540 and a Light Chain Variable Region (VL) comprising the amino acid sequence of SEQ ID NO: 424; or
p) a Heavy Chain Variable Region (VH) comprising the amino acid sequence of SEQ ID NO: 400, or having at least 93%, 94%, 95%, 96%, 97%, 98% or 99% sequence identity to the amino acid sequence of SEQ ID NO: 400 and a Light Chain Variable Region (VL) comprising the amino acid sequence of SEQ ID NO: 414; or
q) a Heavy Chain Variable Region (VH) comprising the amino acid sequence of SEQ ID NO: 410, or having at least 93%, 94%, 95%, 96%, 97%, 98% or 99% sequence identity to the amino acid sequence of SEQ ID NO: 410 and a Light Chain Variable Region (VL) comprising the amino acid sequence of SEQ ID NO: 404; or
r) a Heavy Chain Variable Region (VH) comprising the amino acid sequence of SEQ ID NO: 410, or having at least 93%, 94%, 95%, 96%, 97%, 98% or 99% sequence identity to the amino acid sequence of SEQ ID NO: 410 and a Light Chain Variable Region (VL) comprising the amino acid sequence of SEQ ID NO: 424; or
s) a Heavy Chain Variable Region (VH) comprising the amino acid sequence of SEQ ID NO: 410, or having at least 93%, 94%, 95%, 96%, 97%, 98% or 99% sequence identity to the amino acid sequence of SEQ ID NO: 410 and a Light Chain Variable Region (VL) comprising the amino acid sequence of SEQ ID NO: 434; or
t) a Heavy Chain Variable Region (VH) comprising the amino acid sequence of SEQ ID NO: 420, or having at least 93%, 94%, 95%, 96%, 97%, 98% or 99% sequence identity to the amino acid sequence of SEQ ID NO: 420 and a Light Chain Variable Region (VL) comprising the amino acid sequence of SEQ ID NO: 404; or
u) a Heavy Chain Variable Region (VH) comprising the amino acid sequence of SEQ ID NO: 420, or having at least 93%, 94%, 95%, 96%, 97%, 98% or 99% sequence identity to the amino acid sequence of SEQ ID NO: 420 and a Light Chain Variable Region (VL) comprising the amino acid sequence of SEQ ID NO: 414; or
v) a Heavy Chain Variable Region (VH) comprising the amino acid sequence of SEQ ID NO: 430, or having at least 93%, 94%, 95%, 96%, 97%, 98% or 99% sequence identity to the amino acid sequence of SEQ ID NO: 430 and a Light Chain Variable Region (VL) comprising the amino acid sequence of SEQ ID NO: 404; or
w) a Heavy Chain Variable Region (VH) comprising the amino acid sequence of SEQ ID NO: 430, or having at least 93%, 94%, 95%, 96%, 97%, 98% or 99% sequence identity to the amino acid sequence of SEQ ID NO: 430 and a Light Chain Variable Region (VL) comprising the amino acid sequence of SEQ ID NO: 414; or
x) a Heavy Chain Variable Region (VH) comprising the amino acid sequence of SEQ ID NO: 400, or having at least 93%, 94%, 95%, 96%, 97%, 98% or 99% sequence identity to the amino acid sequence of SEQ ID NO: 400 and a Light Chain Variable Region (VL) comprising the amino acid sequence of SEQ ID NO: 424; or
y) a Heavy Chain Variable Region (VH) comprising the amino acid sequence of SEQ ID NO: 450, or having at least 93%, 94%, 95%, 96%, 97%, 98% or 99% sequence identity to the amino acid sequence of SEQ ID NO: 450 and a Light Chain Variable Region (VL) comprising the amino acid sequence of SEQ ID NO: 424; or
z) a Heavy Chain Variable Region (VH) comprising the amino acid sequence of SEQ ID NO: 480, or having at least 93%, 94%, 95%, 96%, 97%, 98% or 99% sequence identity to the amino acid sequence of SEQ ID NO: 480 and a Light Chain Variable Region (VL) comprising the amino acid sequence of SEQ ID NO: 424; or
aa) a Heavy Chain Variable Region (VH) comprising the amino acid sequence of SEQ ID NO: 520, or having at least 93%, 94%, 95%, 96%, 97%, 98% or 99% sequence identity to the amino acid sequence of SEQ ID NO: 520 and a Light Chain Variable Region (VL) comprising the amino acid sequence of SEQ ID NO: 424; or
bb) a Heavy Chain Variable Region (VH) comprising the amino acid sequence of SEQ ID NO: 430, or having at least 93%, 94%, 95%, 96%, 97%, 98% or 99% sequence identity to the amino acid sequence of SEQ ID NO: 430, and a Light Chain Variable Region (VL) comprising the amino acid sequence of SEQ ID NO: 424.

In one embodiment the ASC binding molecule may comprise:
a) a Heavy Chain Variable Region (VH) comprising the amino acid sequence of SEQ ID NO: 400 and a Light Chain Variable Region (VL) comprising the amino acid sequence of SEQ ID NO: 404; or
b) a Heavy Chain Variable Region (VH) comprising the amino acid sequence of SEQ ID NO: 410 and a Light Chain Variable Region (VL) comprising the amino acid sequence of SEQ ID NO: 414; or
c) a Heavy Chain Variable Region (VH) comprising the amino acid sequence of SEQ ID NO: 420 and a Light Chain Variable Region (VL) comprising the amino acid sequence of SEQ ID NO: 424; or
d) a Heavy Chain Variable Region (VH) comprising the amino acid sequence of SEQ ID NO: 430 and a Light Chain Variable Region (VL) comprising the amino acid sequence of SEQ ID NO: 434; or
e) a Heavy Chain Variable Region (VH) comprising the amino acid sequence of SEQ ID NO: 440 and a Light Chain Variable Region (VL) comprising the amino acid sequence of SEQ ID NO: 434; or
f) a Heavy Chain Variable Region (VH) comprising the amino acid sequence of SEQ ID NO: 450 and a Light Chain Variable Region (VL) comprising the amino acid sequence of SEQ ID NO: 434; or
g) a Heavy Chain Variable Region (VH) comprising the amino acid sequence of SEQ ID NO: 460 and a Light Chain Variable Region (VL) comprising the amino acid sequence of SEQ ID NO: 434; or
h) a Heavy Chain Variable Region (VH) comprising the amino acid sequence of SEQ ID NO: 470 and a Light Chain Variable Region (VL) comprising the amino acid sequence of SEQ ID NO: 434; or
i) a Heavy Chain Variable Region (VH) comprising the amino acid sequence of SEQ ID NO: 480 and a Light Chain Variable Region (VL) comprising the amino acid sequence of SEQ ID NO: 434; or
j) a Heavy Chain Variable Region (VH) comprising the amino acid sequence of SEQ ID NO: 490 and a Light Chain Variable Region (VL) comprising the amino acid sequence of SEQ ID NO: 434; or
k) a Heavy Chain Variable Region (VH) comprising the amino acid sequence of SEQ ID NO: 500 and a Light Chain Variable Region (VL) comprising the amino acid sequence of SEQ ID NO: 434; or
l) a Heavy Chain Variable Region (VH) comprising the amino acid sequence of SEQ ID NO: 510 and a Light Chain Variable Region (VL) comprising the amino acid sequence of SEQ ID NO: 434; or
m) a Heavy Chain Variable Region (VH) comprising the amino acid sequence of SEQ ID NO: 520 and a Light Chain Variable Region (VL) comprising the amino acid sequence of SEQ ID NO: 434; or
n) a Heavy Chain Variable Region (VH) comprising the amino acid sequence of SEQ ID NO: 530 and a Light Chain Variable Region (VL) comprising the amino acid sequence of SEQ ID NO: 424; or
o) a Heavy Chain Variable Region (VH) comprising the amino acid sequence of SEQ ID NO: 540 and a Light Chain Variable Region (VL) comprising the amino acid sequence of SEQ ID NO: 424; or
p) a Heavy Chain Variable Region (VH) comprising the amino acid sequence of SEQ ID NO: 400 and a Light Chain Variable Region (VL) comprising the amino acid sequence of SEQ ID NO: 414; or
q) a Heavy Chain Variable Region (VH) comprising the amino acid sequence of SEQ ID NO: 410 and a Light Chain Variable Region (VL) comprising the amino acid sequence of SEQ ID NO: 404; or
r) a Heavy Chain Variable Region (VH) comprising the amino acid sequence of SEQ ID NO: 410 and a Light Chain Variable Region (VL) comprising the amino acid sequence of SEQ ID NO: 424; or
s) a Heavy Chain Variable Region (VH) comprising the amino acid sequence of SEQ ID NO: 410 and a Light Chain Variable Region (VL) comprising the amino acid sequence of SEQ ID NO: 434; or
t) a Heavy Chain Variable Region (VH) comprising the amino acid sequence of SEQ ID NO: 420 and a Light Chain Variable Region (VL) comprising the amino acid sequence of SEQ ID NO: 404; or
u) a Heavy Chain Variable Region (VH) comprising the amino acid sequence of SEQ ID NO: 420 and a Light Chain Variable Region (VL) comprising the amino acid sequence of SEQ ID NO: 414; or
v) a Heavy Chain Variable Region (VH) comprising the amino acid sequence of SEQ ID NO: 430 and a Light Chain Variable Region (VL) comprising the amino acid sequence of SEQ ID NO: 404; or
w) a Heavy Chain Variable Region (VH) comprising the amino acid sequence of SEQ ID NO: 430 and a Light Chain Variable Region (VL) comprising the amino acid sequence of SEQ ID NO: 414; or
x) a Heavy Chain Variable Region (VH) comprising the amino acid sequence of SEQ ID NO: 400 and a Light Chain Variable Region (VL) comprising the amino acid sequence of SEQ ID NO: 424; or
y) a Heavy Chain Variable Region (VH) comprising the amino acid sequence of SEQ ID NO: 450 and a Light Chain Variable Region (VL) comprising the amino acid sequence of SEQ ID NO: 424; or
z) a Heavy Chain Variable Region (VH) comprising the amino acid sequence of SEQ ID NO: 480 and a Light Chain Variable Region (VL) comprising the amino acid sequence of SEQ ID NO: 424; or
aa) a Heavy Chain Variable Region (VH) comprising the amino acid sequence of SEQ ID NO: 520 and a Light Chain Variable Region (VL) comprising the amino acid sequence of SEQ ID NO: 424; or
bb) a Heavy Chain Variable Region (VH) comprising the amino acid sequence of SEQ ID NO: 430 and a Light Chain Variable Region (VL) comprising the amino acid sequence of SEQ ID NO: 424.

In one embodiment the ASC binding molecule may comprise:
a. a VH-CDR1 comprising the amino acid sequence of SEQ ID NO: 201, a VH-CDR2 comprising the amino acid sequence of SEQ ID NO: 412 and a VH-CDR3 comprising the amino acid sequence SEQ ID NO: 203; a VL-CDR1 comprising the amino sequence SEQ ID NO: 205; a VL-CDR2 comprising the amino sequence SEQ ID NO: 206, and a VL-CDR3 comprising the amino sequence SEQ ID NO: 207; or
b. a VH-CDR1 comprising the amino acid sequence of SEQ ID NO: 201, a VH-CDR2 comprising the amino acid sequence of SEQ ID NO: 462 and a VH-CDR3 comprising the amino acid sequence SEQ ID NO: 203; a VL-CDR1 comprising the amino sequence SEQ ID NO: 435; a VL-CDR2 comprising the amino sequence SEQ ID NO: 206, and a VL-CDR3 comprising the amino sequence SEQ ID NO: 207.
c. a VH-CDR1 comprising the amino acid sequence of SEQ ID NO: 201, a VH-CDR2 comprising the amino acid sequence of SEQ ID NO: 462 and a VH-CDR3 comprising the amino acid sequence SEQ ID NO: 203; a VL-CDR1 comprising the amino sequence SEQ ID NO: 435; a VL-CDR2 comprising the amino sequence SEQ ID NO: 206, and a VL-CDR3 comprising the amino sequence SEQ ID NO: 207.

In one embodiment the ASC binding molecule may comprise:
a. a Heavy Chain Variable Region (VH) comprising the amino acid sequence of SEQ ID NO: 440, or having at least 93%, 94%, 95%, 96%, 97%, 98% or 99% sequence identity to the amino acid sequence of SEQ ID NO: 440 and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
b. a Heavy Chain Variable Region (VH) comprising the amino acid sequence of SEQ ID NO: 460, or having at least 93%, 94%, 95%, 96%, 97%, 98% or 99% sequence identity to the amino acid sequence of SEQ ID NO: 460 and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
c. a Heavy Chain Variable Region (VH) comprising the amino acid sequence of SEQ ID NO: 520, or having at least 93%, 94%, 95%, 96%, 97%, 98% or 99% sequence identity to the amino acid sequence of SEQ ID NO: 520 and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
d. a Heavy Chain Variable Region (VH) comprising the amino acid sequence of SEQ ID NO: 480, or having at least 93%, 94%, 95%, 96%, 97%, 98% or 99% sequence identity to the amino acid sequence of SEQ ID NO: 480 and a Light Chain Variable Region (VL) comprising the amino acid sequence of SEQ ID NO: 424 or a Light Chain Variable Region (VL) having at least 95%, 96%, 97%, 98% or 99% sequence identity to the amino acid sequence of SEQ ID NO: 424.

In one embodiment the ASC binding molecule may comprise:
a. a Heavy Chain Variable Region (VH) comprising the amino acid sequence of SEQ ID NO: 440, or having at least 93%, 94%, 95%, 96%, 97%, 98% or 99% sequence identity to the amino acid sequence of SEQ ID NO: 440 and a Light Chain Variable Region (VL) comprising the amino acid sequence of SEQ ID NO: 434; or
b. a Heavy Chain Variable Region (VH) comprising the amino acid sequence of SEQ ID NO: 460, or having at least 93%, 94%, 95%, 96%, 97%, 98% or 99% sequence identity to the amino acid sequence of SEQ ID NO: 460 and a Light Chain Variable Region (VL) comprising the amino acid sequence of SEQ ID NO: 434; or
c. a Heavy Chain Variable Region (VH) comprising the amino acid sequence of SEQ ID NO: 520, or having at least 93%, 94%, 95%, 96%, 97%, 98% or 99% sequence identity to the amino acid sequence of SEQ ID NO: 520 and a Light Chain Variable Region (VL) comprising the amino acid sequence of SEQ ID NO: 434; or
d. a Heavy Chain Variable Region (VH) comprising the amino acid sequence of SEQ ID NO: 480, or having at least 93%, 94%, 95%, 96%, 97%, 98% or 99% sequence identity to the amino acid sequence of SEQ ID NO: 480 and a Light Chain Variable Region (VL) comprising the amino acid sequence of SEQ ID NO: 424.

In one embodiment the ASC binding molecule may comprise:
a. a Heavy Chain Variable Region (VH) comprising the amino acid sequence of SEQ ID NO: 440 and a Light Chain Variable Region (VL) comprising the amino acid sequence of SEQ ID NO: 434; or
b. a Heavy Chain Variable Region (VH) comprising the amino acid sequence of SEQ ID NO: 460 and a Light Chain Variable Region (VL) comprising the amino acid sequence of SEQ ID NO: 434; or
c. a Heavy Chain Variable Region (VH) comprising the amino acid sequence of SEQ ID NO: 520 and a Light Chain Variable Region (VL) comprising the amino acid sequence of SEQ ID NO: 434; or
d. a Heavy Chain Variable Region (VH) comprising the amino acid sequence of SEQ ID NO: 480 and a Light Chain Variable Region (VL) comprising the amino acid sequence of SEQ ID NO: 424.

In one preferred embodiment the ASC binding molecule may comprise a VH-CDR1 comprising the amino acid sequence of SEQ ID NO: 201, a VH-CDR2 comprising the amino acid sequence of SEQ ID NO: 412 and a VH-CDR3 comprising the amino acid sequence SEQ ID NO: 203; a VL-CDR1 comprising the amino sequence SEQ ID NO: 205; a VL-CDR2 comprising the amino sequence SEQ ID NO: 206, and a VL-CDR3 comprising the amino sequence SEQ ID NO: 207.

In one preferred embodiment the ASC binding molecule may comprise a VH-CDR1 comprising the amino acid sequence of SEQ ID NO: 201, a VH-CDR2 comprising the amino acid sequence of SEQ ID NO: 462 and a VH-CDR3 comprising the amino acid sequence SEQ ID NO: 203; a VL-CDR1 comprising the amino sequence SEQ ID NO: 435; a VL-CDR2 comprising the amino sequence SEQ ID NO: 206, and a VL-CDR3 comprising the amino sequence SEQ ID NO: 207.

In one preferred embodiment the ASC binding molecule may comprise a VH-CDR1 comprising the amino acid sequence of SEQ ID NO: 201, a VH-CDR2 comprising the amino acid sequence of SEQ ID NO: 462 and a VH-CDR3 comprising the amino acid sequence SEQ ID NO: 203; a VL-CDR1 comprising the amino sequence SEQ ID NO: 435; a VL-CDR2 comprising the amino sequence SEQ ID NO: 206, and a VL-CDR3 comprising the amino sequence SEQ ID NO: 207

In one embodiment the ASC binding molecule may comprise a Heavy Chain Variable Region (VH) comprising the amino acid sequence of SEQ ID NO: 440, or having at least 93%, 94%, 95%, 96%, 97%, 98% or 99% sequence identity to the amino acid sequence of SEQ ID NO: 440 and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434.

In one embodiment the ASC binding molecule may comprise a Heavy Chain Variable Region (VH) comprising the amino acid sequence of SEQ ID NO: 460, or having at least 93%, 94%, 95%, 96%, 97%, 98% or 99% sequence identity to the amino acid sequence of SEQ ID NO: 460 and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434.

In one embodiment the ASC binding molecule may comprise a Heavy Chain Variable Region (VH) comprising the amino acid sequence of SEQ ID NO: 520, or having at least 93%, 94%, 95%, 96%, 97%, 98% or 99% sequence identity to the amino acid sequence of SEQ ID NO: 520 and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434.

In one embodiment the ASC binding molecule may comprise a Heavy Chain Variable Region (VH) comprising the amino acid sequence of SEQ ID NO: 480, or having at least 93%, 94%, 95%, 96%, 97%, 98% or 99% sequence identity to the amino acid sequence of SEQ ID NO: 480 and a Light Chain Variable Region (VL) comprising the amino acid sequence of SEQ ID NO: 424 or a Light Chain Variable Region (VL) having at least 95%, 96%, 97%, 98% or 99% sequence identity to the amino acid sequence of SEQ ID NO: 424.

In one embodiment the ASC binding molecule may comprise a Heavy Chain Variable Region (VH) comprising the amino acid sequence of SEQ ID NO: 440 and a Light Chain Variable Region (VL) comprising the amino acid sequence of SEQ ID NO: 434.

In one embodiment the ASC binding molecule may comprise a Heavy Chain Variable Region (VH) comprising the amino acid sequence of SEQ ID NO: 460 and a Light Chain Variable Region (VL) comprising the amino acid sequence of SEQ ID NO: 434.

In one embodiment the ASC binding molecule may comprise a Heavy Chain Variable Region (VH) comprising the amino acid sequence of SEQ ID NO: 520 and a Light Chain Variable Region (VL) comprising the amino acid sequence of SEQ ID NO: 434.

In one embodiment the ASC binding molecule may comprise a Heavy Chain Variable Region (VH) comprising the amino acid sequence of SEQ ID NO: 480 and a Light Chain Variable Region (VL) comprising the amino acid sequence of SEQ ID NO: 424.

The ASC binding molecule may be a heterohybrid anti-ASC antibody or an antigen binding fragment thereof. The heterohybrid anti-ASC antibody may optionally be a humanized anti-ASC antibody or chimeric anti-ASC antibody.

The ASC binding molecule may be a monoclonal antibody or an antigen binding fragment thereof. Optionally, the heterohybrid anti-ASC antibody may be a monoclonal antibody. For example, the ASC binding molecule may be a humanized anti-ASC antibody that binds to ASC speck and/or non-polymerized ASC.

The ASC binding molecule may be a heterohybrid anti-ASC antibody or an antigen binding fragment thereof. The heterohybrid anti-ASC antibody may optionally be a humanized anti-ASC antibody or chimeric anti-ASC antibody.

The ASC binding molecule may be a monoclonal antibody or an antigen binding fragment thereof. Optionally, the heterohybrid anti-ASC antibody may be a monoclonal antibody. For example, the ASC binding molecule may be a humanized anti-ASC antibody that binds to ASC speck and/or non-polymerized ASC.

The ASC binding molecule may exhibit an affinity constant, KD, in the range of from about 49 μM to about 1010 μM for human ASC. The ASC binding molecule may additionally exhibit an association rate, ka, value in the range of from about 2.09E+04 1/Ms to about 1.08E+05 1/Ms for human ASC.

Further additionally, the ASC binding molecule may exhibit a dissociation rate, kd, value in the range of from about 4.82E-06 1/s to about 2.11E-05 1/s for human ASC.

The equilibrium dissociation constant (KD), the dissociation rate constant (kd) and the association rate constant (ka) values may be determined by surface plasmon resonance.

The ASC binding molecule, which may be an antibody or an antibody-binding fragment thereof, may be selected from Table 20. For example, the antibody or an antibody-binding fragment thereof, may comprise the sequence defined by ACI-8016-32B6C7-AB1, ACI-8016-2629E8D1-AB1, ACI-8016-2504F3D9-AB1, ACI-8016-18F4C12-AB1, ACI-8016-2622E12F11-AB1, ACI-8016-2609F4A9-AB1.

The antibody or an antibody-binding fragment thereof, may comprise the sequence defined by ACI-8016-32B6C7-AB1.

The antibody or an antibody-binding fragment thereof, may comprise the sequence defined by ACI-8016-2629E8D1-AB1.

The antibody or an antibody-binding fragment thereof, may comprise the sequence defined by ACI-8016-2504F3D9-AB1.

The antibody or an antibody-binding fragment thereof, may comprise the sequence defined by ACI-8016-18F4C12-AB1.

The antibody or an antibody-binding fragment thereof, may comprise the sequence defined by ACI-8016-2622E12F11-AB1.

The antibody or an antibody-binding fragment thereof, may comprise the sequence defined by ACI-8016-2609F4A9-AB1.

The ASC binding molecule may be an antibody or an antibody-binding fragment thereof according to Table 19 or Table 20. For example, the antibody or an antibody-binding fragment thereof, may comprise the sequence defined by hACI-8016-32B6C7-AB1_H5L4, hACI-8016-32B6C7-AB1_H7L4, hACI-8016-32B6C7-AB1_H13L4 or hACI-8016-32B6C7-AB1_H9L3.

For example, according to one embodiment, based on KD, titers and functionality indicating a favorable developability profile, an antibody or an antibody-binding fragment thereof of the sequence defined by hACI-8016-32B6C7-AB1_H5L4, hACI-8016-32B6C7-AB1_H7L4, hACI-8016-32B6C7-AB1_H13L4 or hACI-8016-32B6C7-AB1_H9L3 may be preferred.

In one embodiment the antibody or an antibody-binding fragment thereof, may comprise the sequence defined by hACI-8016-32B6C7-AB1_H5L4.

In one embodiment the antibody or an antibody-binding fragment thereof, may comprise the sequence defined by hACI-8016-32B6C7-AB1_H7L4. In one embodiment the antibody or an antibody-binding fragment thereof, may comprise the sequence defined by hACI-8016-32B6C7-AB1_H13L4. In one embodiment the antibody or an antibody-binding fragment thereof, may comprise the sequence defined by hACI-8016-32B6C7-AB1_H9L3.

In one embodiment, binding affinity to ASC, for example ASC specks and/or non-polymerized ASC may be evaluated by determining the equilibrium dissociation constant (KD, also referred to as the affinity constant or the dissociation constant) using surface plasmon resonance (SPR; Biacore 8K, GE Healthcare Life Sciences). Reference may be made to Examples 11 for a detailed description of suitable SPR methods that may be employed.

The ASC binding molecule, in particular a heterohybrid anti-ASC antibody or antigen binding fragment thereof, may have an equilibrium dissociation constant (KD) of <10 nM, ≤1 nM, ≤100 μM, ≤10p M, or ≤1 μM, (e.g. from 10-8 or less, e.g. from 10-8 M to 10-13 M, e.g. from 10-9 M to 10-11 M), in particular with respect to binding ASC, in particular human ASC. For example, the heterohybrid anti-ASC antibody of the invention may have a KD for human ASC of 2000 μM or less, in specific embodiments 1500 μM or less, such as 1250 μM or less and preferably 1050 μM or less. This is demonstrated for ASC binding molecules of the invention in Example 11 with reference to Table 21.

The ASC binding molecules of the invention may have a KD for human ASC of 1050 μM or less, a KD for human ASC of 150 μM or less, a KD for human ASC of 100 μM or less, a KD for human ASC of 90 μM or less, a KD for human ASC of 80 μM or less, a KD for human ASC of 70 μM or less, a KD for human ASC of 60 μM or less, or a KD for human ASC of 50 μM or less.

The ASC binding molecules of the invention may have a dissociation rate (kd) for human ASC of 9.5E-06 1/s or less, a dissociation rate (kd) for human ASC of 9.0E-06 1/s or less, a dissociation rate (kd) for human ASC of 8.5E-06 1/s or less, a dissociation rate (kd) for human ASC of 8E-06 1/s or less, a dissociation rate (kd) for human ASC of 7.5E-06 1/s or less, a dissociation rate (kd) for human ASC of 7E-06 1/s or less, a dissociation rate (kd) for human ASC of 6.5E-06 1/s or less, a dissociation rate (kd) for human ASC of 6E-06 1/s or less, a dissociation rate (kd) for human ASC of 5.5E-06 1/s or less, a dissociation rate (kd) for human ASC of 5E-06 1/s or less, a dissociation rate (kd) for human ASC of 4.5E-06 1/s or less, a dissociation rate (kd) for human ASC of 4E-06 1/s or less, a dissociation rate (kd) for human ASC of 3.5E-05 1/s or less.

The ASC binding molecules of the invention may have an association rate (ka) for human ASC of 2E+5 1/Ms or less, an association rate (ka) of 1.5E+5 1/Ms or less, an association rate (ka) of 1E+5 1/Ms or less, an association rate (ka) of 9.5E+4 1/Ms or less, an association rate (ka) of 9E+4 1/Ms or less, an association rate (ka) of 8.5E+4 1/Ms or less, an association rate (ka) of 8E+4 1/Ms or less, an association rate (ka) of 7.5E+4 1/Ms or less, an association rate (ka) of 7E+4 1/Ms or less, an association rate (ka) of 6.5E+4 1/Ms or less, an association rate (ka) of 6E+4 1/Ms or less, an association rate (ka) of 5.5E+4 1/Ms or less, an association rate (ka) of 5E+4 1/Ms or less, an association rate (ka) of 4.5E+4 1/Ms or less, an association rate (ka) of 4E+4 1/Ms or less, an association rate (ka) of 3.5E+4 1/Ms or less, an association rate (ka) of 3E+4 1/Ms or less.

In some embodiments, the ASC binding molecule may exhibit an equilibrium dissociation constant, KD, in the range of from about 40 μM to about 1020 μM for human ASC. The ASC binding molecule may additionally exhibit an association rate, ka, value in the range of from about 2.0E+04 1/Ms to about 1.0E+05 1/Ms for human ASC. Further additionally, the ASC binding molecule may exhibit a dissociation rate, kd, value in the range of from about 4.0E-06 1/s to about 2.0E-05 1/s for human ASC. The equilibrium dissociation constant (KD), the dissociation rate constant (kd) and the association rate constant (ka) values may be determined by surface plasmon resonance.

The ASC binding molecule, which may be an antibody or an antibody-binding fragment thereof, may comprise the sequence defined by ACI-8016-32B6C7-AB1, ACI-8016-2629E8D1-AB1, ACI-8016-2504F3D9-AB1, ACI-8016-18F4C12-AB1, ACI-8016-2622E12F11-AB1, ACI-8016-2609F4A9-AB1 as set forth in Table 20.

In some embodiments, the ASC binding molecule of the invention for use in human or veterinary therapy. In one embodiment, the ASC binding molecule for use of the invention is for the prevention, alleviation, treatment and/or diagnosis of a disease, disorder or condition associated with ASC dependent inflammation activation. In one embodiment, the ASC binding molecule for use of the invention is for the prevention, alleviation, treatment and/or diagnosis of a disease, disorder or condition associated with accumulation of ASC and/or ASC specks, preferably extracellular ASC specks. In one embodiment, the ASC binding molecule of the invention, is for use in the prevention of a disease, disorder or condition associated with accumulation of ASC and/or ASC specks, preferably extracellular ASC specks. In one embodiment, the ASC binding molecule or immunoconjugate of the invention is for use in postponing the onset of a disease, disorder or condition associated with accumulation of ASC and/or ASC specks, preferably extracellular ASC specks. In one embodiment, the ASC binding molecule of the invention, is for use in the alleviation of a disease, disorder or condition associated with accumulation of ASC and/or ASC specks, preferably extracellular ASC specks. In one embodiment the ASC binding molecule of the invention is, for use in the treatment of a disease, disorder or condition associated with accumulation of non-polymerized ASC or ASC specks, preferably ASC specks, more preferably extracellular non-polymerized ASC and/or extracellular ASC specks. In one embodiment, the ASC binding molecule or immunoconjugate of the invention is for use in the prevention, alleviation or treatment of a disease, disorder or condition associated with demyelination. In one embodiment, the disease, disorder or condition associated with accumulation of accumulation of ASC or ASC specks, preferably ASC specks, more preferably extracellular ASC specks, is selected from either a central nervous system disease or a peripheral inflammatory condition, The central nervous system disease is preferably Parkinson's disease, Alzheimer disease, multiple sclerosis, amyotrophic lateral sclerosis, traumatic brain injury, spinal cord injury or chronic traumatic encephalopathy. The peripheral inflammatory condition is preferably Non-Alcoholic SteatoHepatitis (NASH), Cryopyrin-associated periodic syndrome (CAPS), Chronic obstructive pulmonary disease (COPD), gout, acne, Hidradenitis Suppurativa (HS), psoriasis, Inflammatory Bowel Disease (IBD) (e.g. ulcerative colitis or Crohn's disease), Edema (DME), Geographic Atrophy (GA), Coronavirus-associated respiratory distress syndrome (CARDS) or Sjogren's Syndrome.

In additional embodiments, the ASC binding molecule, particularly an anti-ASC antibody or an antigen-binding fragment thereof, of the invention is envisaged for prevention, alleviation, treatment and/or diagnosis of a disease, disorder or condition associated with accumulation of ASC or ASC specks, preferably ASC specks, more preferably extracellular ASC specks, or diseases involving inflammasome activation.

In one embodiment, the method of the invention comprises the postponement of the onset of a disease, disorder or condition associated with accumulation of ASC and/or ASC specks, preferably extracellular ASC specks.

In one embodiment, the method of the invention comprises the prevention, alleviation or treatment of a disease, disorder or condition associated with demyelination.

Diseases envisaged according to the present invention include Central Nervous Disease (CNS), pain, lung and airway diseases, cardiovascular diseases, liver diseases, metabolic and renal diseases, skin diseases, reproductive disorders, autoinflammatory and autoimmune diseases, cancers, infectious diseases and peripheral inflammatory conditions.

CNS diseases may be Parkinson's disease, Alzheimer's disease, Age-related cognitive impairment, mild cognitive impairment, Frontotemporal dementia, amyotrophic lateral sclerosis, Traumatic brain injury, chronic traumatic encephalopathy, spinal cord injury, Stroke, Intracerebral hemorrhage, multiple sclerosis, Sepsis-associated encephalopathy, Cerebral ischemia, Subarachnoid hemorrhage, Epilepsy, Acrylamide poisoning, Opioid-induced neuroinflammation, Chronic migraine, Perioperative neurocognitive disorders, Poststroke cognitive impairment, Post-cardiac arrest cognitive impairment, Social isolation-induced cognitive impairment, Anxiety, Multiple System Atrophy, Pick disease, Progressive isolated aphasia, or Lewy body dementia and post-traumatic stress disorder. Preferably, the CNS disease is Parkinson's Disease, Alzheimer's disease, multiple sclerosis, amyotrophic lateral sclerosis, traumatic brain injury, spinal cord injury, chronic traumatic encephalopathy.

Pain may be neuropathic pain.

Lung and airway diseases may be Allergic rhinitis, Chronic obstructive pulmonary disease, Cystic fibrosis, Acute Respiratory Distress Syndrome, Steroid-resistant asthma, Asthma, ischemia reperfusion lung injury, Particulate matter-induced lung injury, Radiation pneumonitis, Pulmonary hypertension, Sarcoidosis. Cardiovascular diseases may be Atherosclerosis, Heart failure, Hypertension, Myocardial infarction, atrial fibrillation, Cardiac injury induced by metabolic dysfunction, Heart failure, Endothelial dysfunction. Gastrointestinal diseases such colitis, inflammatory bowel disease.

Liver diseases may be Acute liver failure, Circadian regulation of immunity, Non-Alcoholic SteatoHepatitis (NASH), ischemia reperfusion liver injury, Idiosyncratic drug-induced liver injury, Liver fibrosis.

Metabolic and renal diseases may be Diabetic encephalopathy, Diabetes-associated atherosclerosis, Insulin resistance, Islet transplantation rejection, Chronic crystal nephropathy, Renal fibrosis, ischemia/reperfusion kidney injury, Obesity-associated renal disease, Renal hypertension, Focal Segmental Glomerulo Sclerosis, diabetic nephropathy, IgA nephropathy.

Skin diseases may be psoriasis, acne, hidradenitis suppurativa.

Reproductive disorders may be Preterm birth.

Autoinflammatory and autoimmune diseases may be Familial Mediterranean fever, Cryopyrin-associated periodic syndrome (CAPS), Schnitzler syndrome, Myelodysplastic syndromes), Rheumatoid Arthritis, Sickle cell disease, valosin containing protein (VCP)-associated disease, gout, Systemic lupus erythematosus, psoriatic arthritis).

Infectious diseases may be caused by bacteria, Viruses or parasites, such as Human Immunodeficiency Virus-1 (HIV-1), CoronaVirus Disease (COVID)-19, Hepatitis B.

Peripheral inflammatory conditions may be Non-Alcoholic SteatoHepatitis (NASH), Cryopyrin-associated periodic syndrome (CAPS), Chronic obstructive pulmonary disease (COPD), gout, acne, Hidradenitis Suppurativa (HS), Inflammatory Bowel Disease (IBD) (e.g. ulcerative colitis or Crohn's disease), Edema (DME), Geographic Atrophy (GA), Coronavirus-associated respiratory distress syndrome (CARDS) or Sjogren's Syndrome.

In one embodiment, the method of the invention comprises the prevention or reduction of demyelination in a subject. The method may comprise administering the ASC binding molecule described herein or the immunoconjugate described herein to the subject. In one embodiment, the prevention or reduction of demyelination is improving demyelination score in vivo.

In one embodiment, the method of the invention comprises the reduction of levels of reactive microglia in a subject. The method may comprise administering the ASC binding molecule described herein or the immunoconjugate described herein to the subject.

In one embodiment, the method of the invention comprises the reduction of levels of ASC and/or cleaved capase-1 protein in a subject. The method may comprise administering the ASC binding molecule described herein or the immunoconjugate described herein to the subject.

In one embodiment, the method of the invention comprises the reduction of levels of infiltrating CD4+ T-cells in the spinal cord of a subject. The method may comprise administering the ASC binding molecule described herein or the immunoconjugate described herein to the subject.

The invention also relates to compositions comprising an ASC binding molecule, particularly an anti-ASC antibody or an antigen-binding fragment thereof, of the invention as described herein. The invention furthermore relates to immunotherapeutic and/or immunodiagnostic methods using such compositions in the prevention, diagnosis and/or treatment of a ASC-speck associated disease, disorder or condition, wherein an effective amount of the composition is administered to a subject in need thereof.

In some embodiments, the invention encompasses ASC binding molecules, particularly anti-ASC antibodies and antigen-binding fragments thereof of the invention as described herein that specifically bind ASC and the use of these binding molecules to diagnose, prevent, alleviate and/or treat a disease, disorder or condition associated with accumulation of ASC and/or ASC specks, preferably extracellular ASC specks.

The methods and compositions disclosed herein have applications in diagnosing, preventing, alleviating and/or treating a disease, disorder or condition associated with accumulation of ASC and/or ASC specks, preferably extracellular ASC specks.

In another embodiment, an ASC binding molecule, particularly anti-ASC antibody or an antigen-binding fragment thereof of the invention as described herein is contacted with a sample to detect, diagnose and/or monitor a disease, disorder or condition associated with accumulation of ASC and/or ASC specks, preferably extracellular ASC specks.

In one embodiment, the invention encompasses ASC binding molecules, particularly anti-ASC antibodies or antigen-binding fragments thereof of the invention as described herein that specifically bind ASC specks and/or non-polymerized ASC and the use of these molecules, particularly of these antibodies, to detect the presence of ASC in a sample. Accordingly, ASC binding molecules, particularly anti-ASC antibodies or antigen-binding fragments thereof of the invention, can be used, inter alia, to screen a clinical sample, in particular a body fluid, particularly human blood, CSF, interstitial fluid (ISF) and/or urine for the presence of ASC in samples, for example, by using an ELISA-based or surface adapted assay. The methods and compositions of the invention also have applications in diagnosing presymptomatic disease and/or in monitoring disease progression and/or therapeutic efficacy. Many suitable immunoassay formats are known. Thus, the methods (such as ELISA, MSD (Meso Scale Discovery), HTRF (Homogeneous Time Resolved Fluorescence) and AlphaLISA) may be performed for diagnostic purposes. Alternatively, the methods may be performed for monitoring purposes. Increased levels over time may indicate progression of the disease. Decreased levels over time may indicate regression of the disease. The methods may also be used to monitor therapy, in particular to monitor the efficacy of a particular treatment. Methods of quantifying ASC in suitable samples using binding molecules of the invention may also be used to select a therapy (for further treatment of the subject). Thus, personalized treatment methods are envisaged. In preferred embodiments, the therapy comprises ASC binding molecules, particularly anti-ASC antibodies or antigen-binding fragments of the invention, typically in the form of a pharmaceutical composition as described herein.

In other embodiments, the invention provides methods for preventing, alleviating and/or treating a disease, disorder or condition associated with ASC dependent inflammasomes. According to one embodiment, the methods of the invention comprise administering an effective concentration of an ASC binding molecule, particularly anti-ASC antibody or antigen-binding fragment thereof of the invention specific for ASC as described herein to a subject. In another embodiment, the invention provides a method for preventing, alleviating and/or treating an inflammasome associated disease. According to some embodiments, an ASC binding molecule, particularly an anti-ASC antibody of the invention or an antigen-binding fragment thereof as described herein specific for ASC is administered to treat, alleviate and/or prevent a disease defined herein.

In some embodiments, an immunoconjugate is provided, wherein the immunoconjugate comprises an (isolated) antibody described herein and a therapeutic agent. In some embodiments, a labeled antibody is provided, comprising an antibody described herein and a detectable label.

In some embodiments, a pharmaceutical composition is provided, comprising an (isolated) antibody described herein and a pharmaceutically acceptable carrier.

In some embodiments the ASC binding molecule, particularly anti-ASC antibody or antigen binding fragment thereof of the present invention is linked to a detectable label.

In some embodiments the ASC binding molecule, particularly anti-ASC antibody or antigen binding fragment thereof is part of an immunoconjugate wherein the ASC binding molecule, particularly anti-ASC antibody or antigen binding fragment thereof is covalently linked to another suitable therapeutic agent.

In some embodiments, the ASC binding molecule, particularly anti-ASC antibody or antigen binding fragment thereof or the immunoconjugate comprising it is present as a composition comprising an ASC binding molecules, particularly anti-ASC antibody or antigen binding fragment thereof.

In some embodiments the ASC binding molecules, particularly anti-ASC antibody or antigen binding fragment thereof is part of pharmaceutical composition comprising an ASC binding molecule, particularly an anti-ASC antibody or antigen binding fragment thereof, or an immunoconjugate wherein the ASC binding molecules, particularly anti-ASC antibody or antigen binding fragment thereof is covalently linked to another suitable therapeutic agent, or a composition as herein described.

In some embodiments the ASC binding molecules, particularly anti-ASC antibody or antigen binding fragment thereof is part of a detection and/or diagnostic kit comprising an ASC binding molecules, particularly anti-ASC antibody or antigen binding fragment thereof, or an immunoconjugate wherein the ASC binding molecule, particularly anti-ASC antibody or antigen binding fragment thereof is covalently linked to another suitable therapeutic agent, or a composition as herein described.

Kits containing the binding molecules of the invention are also provided. In particular, such kits may be useful for performing the diagnostic methods of the invention (which include classification, monitoring and therapy selection methods). Thus, a kit for diagnosis of a disease, disorder and/or abnormality associated with ASC-dependent inflammasome or for use in a method of the invention is provided comprising an ASC binding molecule, particularly anti-ASC antibody or antigen binding fragment thereof of the invention. Such kits may comprise all necessary components for performing the herein provided methods. Typically, each component is stored separately in a single overall packaging. Suitable additional components for inclusion in the kits are, for example, buffers, detectable dyes, laboratory equipment, reaction containers, instructions and the like. Instructions for use may be tailored to the specific method for which the kit is to be employed. Suitably labelled ASC binding molecule, particularly anti-ASC antibody or antigen binding fragment thereof of the invention are also provided, which may be included in such kits.

In some embodiments the ASC binding molecules, particularly anti-ASC antibody or antigen binding fragment thereof is part of an immunotherapeutic method for the prevention, or treatment of a disease, disorder or condition associated with ASC, in particular associated with accumulation of ASC and/or ASC specks, preferably extracellular ASC specks or ASC-speck complexes that propagate inflammation, wherein an effective amount of the ASC binding molecules, particularly anti-ASC antibody or antigen binding fragment thereof, or an immunoconjugate wherein the ASC binding molecules, particularly anti-ASC antibody or antigen binding fragment thereof is covalently linked to another suitable therapeutic agent, or a composition as described herein is administered to a subject in need thereof.

In some embodiments the ASC binding molecule, particularly anti-ASC antibody or antigen binding fragment thereof, or an immunoconjugate wherein the ASC binding molecule, particularly anti-ASC antibody or antigen binding fragment thereof is covalently linked to another suitable therapeutic agent, or a composition as described herein is administered to a subject in need thereof is used to diagnose, prevent, alleviate or treat a disease, disorder or condition associated with ASC, in particular associated with accumulation of ASC and/or ASC specks, preferably extracellular ASC specks.

In other embodiments, the invention relates to any methods for detecting, diagnosing or monitoring a a disease, disorder or condition associated with ASC, in particular associated with ASC and/or ASC specks, preferably extracellular ASC specks.

Preferably, the disease, disorder or condition associated with ASC, is associated with ASC-speck complexes that propagate inflammation disclosed herein.

In some embodiments the ASC binding molecule, particularly anti-ASC antibody or antigen binding fragment thereof is used in a method for diagnosing presymptomatic disease or for monitoring disease progression and therapeutic efficacy, or for predicting responsiveness, or for selecting subjects which are likely to respond to the treatment with an ASC binding molecule, particularly anti-ASC antibody or antigen binding fragment thereof. Said method may be performed using a sample of human blood or urine. Most preferably the method involves an ELISA-based or surface adapted assay.

In some embodiments the ASC binding molecules, particularly anti-ASC antibody or antigen binding fragment thereof, or an immunoconjugate wherein the ASC binding molecule, particularly anti-ASC antibody or antigen binding fragment thereof is covalently linked to another suitable therapeutic agent, or a composition as described herein is administered to a subject in need thereof is used for manufacturing a medicament for treating a disease, disorder and/or abnormality associated with ASC, in particular associated with associated with accumulation of ASC and/or ASC specks, preferably extracellular ASC specks.

Pharmaceutical formulations of an ASC binding molecules, particularly anti-ASC antibody or antigen binding fragment thereof or immunoconjugate as described herein are prepared by mixing such antibody or immunoconjugate having the desired degree of purity with one or more optional pharmaceutically acceptable carriers (Remington's Pharmaceutical Sciences 16^{th} edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn¬protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX@, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody or immunoconjugate formulations are described in U.S. Pat. No. 6,267,958. Aqueous antibody or immunoconjugate formulations include those described in U.S. Pat. No. 6,171,586 and WO2006/044908, the latter formulations including a histidine-acetate buffer.

The formulation herein may also contain more than one active ingredient as necessary for the particular indication being treated, preferably those with complementary activities that do not adversely affect each other.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16^{th} edition, Osol, A. Ed. (1980).

Sustained-release preparations may be prepared. Suitable examples of sustained-release preparations include semipermeable matrices of solid hydrophobic polymers containing the antibody or immunoconjugate, which matrices are in the form of shaped articles, e.g. films, or microcapsules. The formulations to be used for in vivo administration are generally sterile. Sterility may be readily accomplished, e.g., by filtration through sterile filtration membranes.

Any of the ASC binding molecules, particularly anti-ASC antibody or antigen binding fragment thereof or immunoconjugates provided herein may be used in methods, e.g., therapeutic methods.

In another aspect, an ASC binding molecule, particularly anti-ASC antibody or antigen binding fragment thereof or immunoconjugate for use as a medicament is provided. In further aspects, an ASC binding molecule, particularly anti-ASC antibody or antigen binding fragment thereof or immunoconjugate for use in a method of treatment is provided. In certain embodiments, an ASC binding molecule, particularly anti-ASC antibody or antigen binding fragment thereof or immunoconjugate for use in the prevention, diagnosis and/or treatment of a disease associated with accumulation of ASC and/or ASC specks, preferably extracellular ASC specks. In a preferred embodiment of the invention, ASC binding molecule, particularly anti-ASC antibody or antigen binding fragment thereof or immunoconjugate is provided for use in the prevention, diagnosis and/or treatment of a disease, disorder and/or abnormality associated with ASC, in particular associated with accumulation of ASC and/or ASC specks, preferably extracellular ASC specks.

In a further aspect, the invention provides for the use of an ASC binding molecule, particularly anti-ASC antibody or antigen binding fragment thereof or immunoconjugate in the manufacture or preparation of a medicament. In one such embodiment, the method further comprises administering to the individual an effective amount of at least one additional therapeutic agent, e.g., as described below.

A “subject” or an “individual” according to any of the above embodiments may be an animal, a mammal, preferably a human.

In a further aspect, the invention provides pharmaceutical formulations comprising an ASC binding molecule, particularly anti-ASC antibody or antigen binding fragment thereof or immunoconjugate provided herein, e.g., for use in any of the above therapeutic methods. In one embodiment, a pharmaceutical formulation comprises any of the ASC binding molecules, particularly anti-ASC antibody or antigen binding fragment thereof or immunoconjugates provided herein and a pharmaceutically acceptable carrier. In another embodiment, a pharmaceutical formulation comprises any of the ASC binding molecules, particularly anti-ASC antibody or antigen binding fragment thereof immunoconjugates provided herein and at least one additional therapeutic agent.

Antibodies or immunoconjugates of the invention can be used either alone or in combination with other agents in a therapy. For instance, an ASC binding molecule, particularly anti-ASC antibody or antigen binding fragment thereof or immunoconjugate of the invention may be co-administered with at least one additional therapeutic agent.

Such combination therapies noted above encompass combined administration (where two or more therapeutic agents are included in the same or separate formulations), and separate administration, in which case, administration of the antibody (the preferred type of ASC specific binding molecule) or immunoconjugate of the invention can occur prior to, simultaneously, and/or following, administration of the additional therapeutic agent and/or adjuvant.

An ASC binding molecule, particularly anti-ASC antibody or antigen binding fragment thereof or immunoconjugate of the invention (and any additional therapeutic agent) can be administered by any suitable means, including parenteral, intrapulmonary, and intranasal, and, if desired for local treatment, intralesional, intrauterine or intravesical administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

An ASC binding molecule, particularly anti-ASC antibody or antigen binding fragment thereof or immunoconjugates of the invention would be formulated, dosed, and administered in a fashion consistent with good medical practice. Factors for consideration in this context include the particular disease, disorder and/or abnormality associated with ASC, in particular associated with associated with ASC-speck complexes that propagate inflammation, or the disease being treated, the particular mammal being treated, the clinical condition of the individual subject, the cause of the disease, a disorder and/or abnormality associated with ASC, in particular associated with associated with ASC-speck complexes that propagate inflammation, the site of delivery of the agent, the method of administration, the scheduling of administration, and other factors known to medical practitioners. The ASC binding molecule, particularly anti-ASC antibody or antigen binding fragment thereof or immunoconjugate need not be, but is optionally formulated with one or more agents currently used to prevent or treat the disease, disorder and/or abnormality (referred to interchangeably as a condition) associated with ASC, in particular associated with associated with ASC-speck complexes that propagate inflammation, or the disease in question. The effective amount of such other agents depends on the amount of antibody or immunoconjugate present in the formulation, the type of disease, disorder and/or abnormality associated with ASC, in particular associated with associated with ASC-speck complexes that propagate inflammation or the disease or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an ASC binding molecule, particularly anti-ASC antibody or antigen binding fragment thereof or immunoconjugate of the invention (when used alone or in combination with one or more other additional therapeutic agents) will depend on the type of disease to be treated, the type of antibody or immunoconjugate, the severity and course of the disease, whether the antibody or immunoconjugate is administered for preventive or therapeutic purposes, previous therapy, the subject's clinical history and response to the antibody or immunoconjugate, and the discretion of the attending physician. The ASC binding molecule, particularly anti-ASC antibody or antigen binding fragment thereof or immunoconjugate is suitably administered to the subject at one time or over a series of treatments.

In another aspect of the invention, an article of manufacture containing materials useful for the treatment, prevention and/or diagnosis of a disease, disorder or condition associated with ASC, in particular associated with accumulation of ASC and/or ASC specks, preferably extracellular ASC specks, described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the disease, disorder and/or abnormality associated with ASC, in particular associated with accumulation of ASC and/or ASC specks, preferably extracellular ASC specks, or the disease, and may have a sterile access port (for example the container may be an intravenous solution bag or a vial having a stopper pierceable by a hypodermic injection needle). At least one active agent in the composition is an antibody or immunoconjugate of the invention. The label or package insert indicates that the composition is used for treating the condition of choice.

Moreover, the article of manufacture may comprise (a) a first container with a composition contained therein, wherein the composition comprises an ASC binding molecule, particularly anti-ASC antibody or antigen binding fragment thereof or immunoconjugate of the invention; and (b) a second container with a composition contained therein, wherein the composition comprises a further therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution or dextrose solution. It may further include other materials desirable from a commercial and user standpoint, including other buffers, diluents, filters, needles, and syringes.

In a further embodiment, the invention relates to a method of reducing the level of ASC specks, comprising administering the binding molecule of the invention, the immunoconjugate of the invention, the composition of the invention or the pharmaceutical composition of the invention.

The invention furthermore relates to a method of detecting ASC or ASC specks, comprising contacting a sample with the binding molecule of the invention.

In some embodiments, there is provided a pharmaceutical composition comprising the ASC binding molecule, particularly anti-ASC antibody or an antigen-binding fragment thereof according to the invention and a pharmaceutically acceptable carrier and/or excipient.

In some embodiments, there is provided a nucleic acid molecule encoding an ASC binding molecule, particularly an anti-ASC antibody or an antigen-binding fragment thereof of the invention.

In some embodiments, there is a nucleic acid molecule comprising a nucleotide sequence set forth as:
a. a Heavy Chain Variable Region (VH) encoding sequence of SEQ ID NO: 18 and a Light Chain Variable Region (VL) encoding sequence of SEQ ID NO: 19; or
b. a Heavy Chain Variable Region (VH) encoding sequence of SEQ ID NO: 28 and a Light Chain Variable Region (VL) encoding sequence of SEQ ID NO: 29; or
c. a Heavy Chain Variable Region (VH) encoding sequence of SEQ ID NO: 38 and a Light Chain Variable Region (VL) encoding sequence of SEQ ID NO: 39; or
d. a Heavy Chain Variable Region (VH) encoding sequence of SEQ ID NO: 48 and a Light Chain Variable Region (VL) encoding sequence of SEQ ID NO: 49; or
e. a Heavy Chain Variable Region (VH) encoding sequence of SEQ ID NO: 58 and a Light Chain Variable Region (VL) encoding sequence of SEQ ID NO: 59; or
f. a Heavy Chain Variable Region (VH) encoding sequence of SEQ ID NO: 68 and a Light Chain Variable Region (VL) encoding sequence of SEQ ID NO: 69; or
g. a Heavy Chain Variable Region (VH) encoding sequence of SEQ ID NO: 78 and a Light Chain Variable Region (VL) encoding sequence of SEQ ID NO: 79; or
h. a Heavy Chain Variable Region (VH) encoding sequence of SEQ ID NO: 88 and a Light Chain Variable Region (VL) encoding sequence of SEQ ID NO: 89; or
i. a Heavy Chain Variable Region (VH) encoding sequence of SEQ ID NO: 98 and a Light Chain Variable Region (VL) encoding sequence of SEQ ID NO: 99; or
j. a Heavy Chain Variable Region (VH) encoding sequence of SEQ ID NO: 118 and a Light Chain Variable Region (VL) encoding sequence of SEQ ID NO: 119; or
k. a Heavy Chain Variable Region (VH) encoding sequence of SEQ ID NO: 128 and a Light Chain Variable Region (VL) encoding sequence of SEQ ID NO: 129; or
l. a Heavy Chain Variable Region (VH) encoding sequence of SEQ ID NO: 138 and a Light Chain Variable Region (VL) encoding sequence of SEQ ID NO: 139; or
m. a Heavy Chain Variable Region (VH) encoding sequence of SEQ ID NO: 148 and a Light Chain Variable Region (VL) encoding sequence of SEQ ID NO: 149; or
n. a Heavy Chain Variable Region (VH) encoding sequence of SEQ ID NO: 158 and a Light Chain Variable Region (VL) encoding sequence of SEQ ID NO: 159; or
o. a Heavy Chain Variable Region (VH) encoding sequence of SEQ ID NO: 168 and a Light Chain Variable Region (VL) encoding sequence of SEQ ID NO: 169; or
p. a Heavy Chain Variable Region (VH) encoding sequence of SEQ ID NO: 178 and a Light Chain Variable Region (VL) encoding sequence of SEQ ID NO: 179; or
q. a Heavy Chain Variable Region (VH) encoding sequence of SEQ ID NO: 188 and a Light Chain Variable Region (VL) encoding sequence of SEQ ID NO: 189; or
r. a Heavy Chain Variable Region (VH) encoding sequence of SEQ ID NO: 198 and a Light Chain Variable Region (VL) encoding sequence of SEQ ID NO: 199; or
s. a Heavy Chain Variable Region (VH) encoding sequence of SEQ ID NO: 208 and a Light Chain Variable Region (VL) encoding sequence of SEQ ID NO: 209; or
t. a Heavy Chain Variable Region (VH) encoding sequence of SEQ ID NO: 218 and a Light Chain Variable Region (VL) encoding sequence of SEQ ID NO: 219; or
u. a Heavy Chain Variable Region (VH) encoding sequence of SEQ ID NO: 228 and a Light Chain Variable Region (VL) encoding sequence of SEQ ID NO: 229; or
v. a Heavy Chain Variable Region (VH) encoding sequence of SEQ ID NO: 238 and a Light Chain Variable Region (VL) encoding sequence of SEQ ID NO: 239; or
w. a Heavy Chain Variable Region (VH) encoding sequence of SEQ ID NO: 248 and a Light Chain Variable Region (VL) encoding sequence of SEQ ID NO: 249; or
x. a Heavy Chain Variable Region (VH) encoding sequence of SEQ ID NO: 258 and a Light Chain Variable Region (VL) encoding sequence of SEQ ID NO: 259; or
y. a Heavy Chain Variable Region (VH) encoding sequence of SEQ ID NO: 268 and a Light Chain Variable Region (VL) encoding sequence of SEQ ID NO: 269; or
z. a Heavy Chain Variable Region (VH) encoding sequence of SEQ ID NO: 278 and a Light Chain Variable Region (VL) encoding sequence of SEQ ID NO: 279; or
aa. a Heavy Chain Variable Region (VH) encoding sequence of SEQ ID NO: 288 and a Light Chain Variable Region (VL) encoding sequence of SEQ ID NO: 289; or
bb. a Heavy Chain Variable Region (VH) encoding sequence of SEQ ID NO: 298 and a Light Chain Variable Region (VL) encoding sequence of SEQ ID NO: 299; or
cc. a Heavy Chain Variable Region (VH) encoding sequence of SEQ ID NO: 308 and a Light Chain Variable Region (VL) encoding sequence of SEQ ID NO: 309; or
dd. a Heavy Chain Variable Region (VH) encoding sequence of SEQ ID NO: 318 and a Light Chain Variable Region (VL) encoding sequence of SEQ ID NO: 319; or
ee. a Heavy Chain Variable Region (VH) encoding sequence of SEQ ID NO: 328 and a Light Chain Variable Region (VL) encoding sequence of SEQ ID NO: 329; or
ff. a Heavy Chain Variable Region (VH) encoding sequence of SEQ ID NO: 338 and a Light Chain Variable Region (VL) encoding sequence of SEQ ID NO: 339; or
gg. a Heavy Chain Variable Region (VH) encoding sequence of SEQ ID NO: 348 and a Light Chain Variable Region (VL) encoding sequence of SEQ ID NO: 349; or
hh. a Heavy Chain Variable Region (VH) encoding sequence of SEQ ID NO: 358 and a Light Chain Variable Region (VL) encoding sequence of SEQ ID NO: 359; or
ii. a Heavy Chain Variable Region (VH) encoding sequence of SEQ ID NO: 368 and a Light Chain Variable Region (VL) encoding sequence of SEQ ID NO: 369; or
jj. a Heavy Chain Variable Region (VH) encoding sequence of SEQ ID NO: 378 and a Light Chain Variable Region (VL) encoding sequence of SEQ ID NO: 379; or
kk. a Heavy Chain Variable Region (VH) encoding sequence of SEQ ID NO: 388 and a Light Chain Variable Region (VL) encoding sequence of SEQ ID NO: 389; or
ll. a Heavy Chain Variable Region (VH) encoding sequence of SEQ ID NO: 398 and a Light Chain Variable Region (VL) encoding sequence of SEQ ID NO: 399.

The invention also relates to antibodies that compete for binding to ASC with the antibodies defined above by reference to their amino acid sequence. Thus, those antibodies bind to the same epitope as the antibody with which they compete for binding. Suitable competition assays are described herein and known to those skilled in the art.

In some embodiments, a(n isolated) nucleic acid is provided, wherein the (isolated) nucleic acid comprises SEQ ID NO:18 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:19 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:28 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:29 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:38 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:39 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:48 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:49 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:58 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:59 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:68 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:69 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:78 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:79 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:88 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:89 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:98 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:99 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:118 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:119 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:128 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:129 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:138 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:139 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:148 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:149 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:158 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:159 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:168 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:169 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:178 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:179 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:188 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:189 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:198 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:199 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:208 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:209 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:218 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:219 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:228 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:229 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:238 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:239 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:248 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:249 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:258 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:259 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:268 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:269 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:278 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:279 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:288 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:289 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:298 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:299 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:308 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:309 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:318 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:319 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:328 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:329 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:338 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:339 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:348 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:349 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:358 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:359 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:368 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:369 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:378 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:379 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:388 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:389 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:398 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:399 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:408 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:409 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:418 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:419 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:428 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:429 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:438 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:439 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:448 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:458 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:468 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:478 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:488 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:498 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:508 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:518 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO: 528 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:538 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:548 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:558 encoding an ASC binding molecule, particularly an anti-ASC antibody or an antigen-binding fragment thereof.

In some embodiments, a(n isolated) nucleic acid is provided, wherein the (isolated) nucleic acid comprises SEQ ID NO:559 encoding an ASC binding molecule, particularly an anti-ASC antibody or an antigen-binding fragment thereof.

In certain embodiments, an antibody from a hybridoma clone provided herein is selected from 416E6G4, 402H11C9, 203B12C3, 421B10C12D2, 417E12A8, 413G10A5, 407E10A9, 203G8B10, 401H9B7, 1112B3D7, 2221B7F1, 2314F6H11, 207E8B2, 936.2A1B12, 936.17H1G2, 936.18F4C12, 936.23E5F7, 936.26A1G2, 936.32B6C7, 936.22D3A6, 936.31F10C5, 936.19E6D4, 936.3E6B11, 936.11A3F3, 936.14G5B8, 936.27A1G4, 936.29C5E11, 936.7G3B5, 2504F3D9, 2516A8C6, 2602H6F10, 2609F4A9, 2610H7D3, 2614C3B2, 2617C3A8, 2622E12F11, 2626B9D3 and 2629E8D1.

In a preferred embodiment the antibody from a hybridoma clone provided herein is 936.32B6C7.

The anti-ASC antibody or an antigen-binding fragment thereof is envisaged to treat or prevent diseases. In some embodiments, binding affinity of the ASC binding molecule to human ASC and/or mouse ASC may be evaluated by determining the dissociation constants (KD) using surface plasmon resonance (SPR; Biacore T200, GE Healthcare Life Sciences). Reference may be made to Example 4 for a detailed description of suitable SPR methods that may be employed.

In some embodiments, ASC binding molecules, in particular antibodies or antigen-binding fragments thereof of the invention, typically bind human ASC and/or mouse ASC with high affinity. They may show cross reactivity to human ASC with an EC50 value between 0.05 and 0.27 nM. They may show cross-reactivity to mouse ASC with EC50 in the range 0.07 and 4.52 nM. They may show an EC50 of 5 nm or less, 1 nm or less, 0.5 nm or less or 0.05 nm or less. Reference may be made to Example 1 for a suitable assay.

In some embodiments, the invention provides an ASC antibody of antigen binding fragment thereof that binds a PYD epitope defined by Bin1 in Table 7.

In some embodiments, the invention provides an ASC antibody of antigen binding fragment thereof that binds a PYD epitope defined by Bin2 in Table 7.

In some embodiments, the invention provides an ASC antibody of antigen binding fragment thereof that binds a PYD epitope defined by Bin3 in Table 7.

In some embodiments, the invention provides an ASC antibody of antigen binding fragment thereof that binds a CARD epitope defined by Bin1 in Table 7.

In some embodiments, the invention provides an ASC antibody of antigen binding fragment thereof that binds a CARD epitope defined by Bin2 in Table 7.

In some embodiments, the invention provides an ASC antibody of antigen binding fragment thereof that binds a CARD epitope defined by Bin3 in Table 7.

In some embodiments, the invention provides an ASC binding molecule, that competes for binding to an ASC PYD epitope with any one of the antibodies defined in Table 7, Bin 1, 2 or 3 (PYD). In some embodiments, the invention provides an ASC binding molecule, that competes for binding to an ASC CARD epitope with any one of the antibodies defined in Table 7, Bin 1, 2 or 3 (CARD).

In one embodiment, the ASC binding molecule binds to an epitope in the ASC PYD domain comprising, consisting essentially of, or consisting of amino acid residues:
a) L9, D10, E13, N14, E18 and E19,
b) L9, D10, E13 and N14,
c) E13 and N14,
d) Q79, E80, G83 and Q84; or
e) E18, E19, V30, P31, N71, R74, D75, G77, Q79 and E80.

The amino acids are stated with reference to the amino acid sequence of human ASC of SEQ ID NO: 1. The ASC binding molecule may bind to an epitope in the ASC PYD domain comprising, consisting essentially of, or consisting of amino acid residues numbered:
a) 9, 10, 13, 14, 18 and 19,
b) 9, 10, 13 and 14,
c) 13 and 14,
d) 79, 80, 83 and 84, or
e) 18, 19, 30, 31, 71, 74, 75, 77, 79 and 80
   of human ASC of SEQ ID NO: 1.

Alternatively, the ASC binding molecules may bind to an epitope in the ASC CARD domain comprising, consisting essentially of, or consisting of amino acid residues:
a) K174 and D175,
b) 1115, D116, R119, A120, K174, D175, S184, Q185, S186 and Y187,
c) 1115, D116, N170, W171, T172, K174, D175, S186 and Y187,
d) Y137,
e) R119, A120, L178, Q179, S186 and Y187, or
f) R119, A120, K174, D175, S186 and Y187.

The amino acids are stated with reference to the amino acid sequence of human ASC of SEQ ID NO: 1. The ASC binding molecule may bind to an epitope in the ASC CARD domain comprising, consisting essentially of, or consisting of amino acid residues numbered:
a) 174 and 175,
b) 115, 116, 119, 120, 174, 175, 184, 186 and 187,
c) 115, 116, 170, 171, 172, 174, 175, 186 and 187,
d) 137,
e) 119, 120, 178, 179, 186 and 187 or
f) 119, 120, 174, 175, 186 and 187.
   of human ASC of SEQ ID NO: 1.

The ASC binding molecules, in particular an anti-ASC antibody or antigen-binding fragment thereof, of the invention may be used as detection tools and/or positive controls as they bind to non-polymerized ASC and/or ASC specks in the sample in selective fashion. Diagnostic compositions of the invention may be used in such methods. Mixtures of the invention may be employed in such methods. In some embodiments an ASC binding molecule is part of a diagnostic kit comprising an ASC specific binding molecule, or an immunoconjugate wherein the ASC specific binding molecule is covalently linked to another suitable therapeutic agent, or a composition comprising an ASC specific binding molecule.

In some embodiments an ASC binding molecule is used in an immunodiagnostic method for use in the prevention, diagnosis, alleviation of symptoms associated with, ASC-dependent inflammation or non-polymerized ASC and/or ASC specks.

In some embodiments, a diagnostic composition is provided, comprising an isolated ASC binding molecule, in particular an anti-ASC antibody or antigen-binding fragment thereof, described herein and a pharmaceutically acceptable carrier and/or excipient. Mixtures of the invention may be employed in such diagnostic compositions.

In one embodiment, there is provided a method for diagnosing a disease, disorder and/or condition associated with ASC-dependent inflammation comprising quantifying non-polymerized ASC and/or ASC specks wherein similar or higher levels of non-polymerized ASC and/or ASC specks in the sample compared with a diseased control level are indicative of a disease, disorder and/or condition associated with ASC-dependent inflammation.

In one embodiment, there is provided a method for classifying a disease, disorder and/or condition associated with ASC-dependent inflammation comprising performing the method of quantifying non-polymerized ASC and/or ASC specks; classifying the disease, disorder and/or condition associated with ASC-dependent inflammation.

In one embodiment, there is provided a method for monitoring a disease, disorder and/or condition associated with non-polymerized ASC and/or ASC specks at two or more time points using samples from a subject comprising contacting the samples with an ASC binding antibody or antigen-binding fragment thereof of the invention, wherein;
a. higher levels of non-polymerized ASC and/or ASC specks in the later sample compared with one or more earlier samples are indicative of progression of a disease, disorder and/or condition associated with non-polymerized ASC and/or ASC specks;
b. lower levels of non-polymerized ASC and/or ASC specks in the later sample compared with one or more earlier samples are indicative of regression of a disease, disorder and/or condition associated with non-polymerized ASC and/or ASC specks; and/or
c. no significant change of levels of non-polymerized ASC and/or ASC specks in the later sample compared with one or more earlier samples are indicative of lack of progression of a disease, disorder and/or condition associated with non-polymerized ASC and/or ASC specks.

In one embodiment, there is provided a method for selecting a therapy for treatment of a disease, disorder and/or condition associated with non-polymerized ASC and/or ASC specks comprising contacting samples taken before and after treatment with the therapy with an ASC binding antibody or antigen-binding fragment thereof of the invention, wherein;
a. lower levels of non-polymerized ASC and/or ASC specks in the sample taken after treatment compared with the sample taken before treatment are indicative of successful treatment of a disease, disorder and/or condition associated with non-polymerized ASC and/or ASC specks and thus the therapy is selected for treatment;
b. no significant change of levels of non-polymerized ASC and/or ASC specks in the sample taken after treatment compared with the sample taken before treatment are indicative of successful treatment of a disease, disorder and/or condition associated with non-polymerized ASC and/or ASC specks and thus the therapy is selected for treatment;
c. a decline in the rate of increase of levels of non-polymerized ASC and/or ASC specks between samples taken during treatment compared with samples taken before treatment are indicative of successful treatment of a disease, disorder and/or condition associated with non-polymerized ASC and/or ASC specks and thus the therapy is selected for treatment;
d. higher levels of non-polymerized ASC and/or ASC specks in the sample taken after treatment compared with the sample taken before treatment are indicative of unsuccessful treatment of a disease, disorder and/or condition associated with non-polymerized ASC and/or ASC specks and thus the therapy is not selected for treatment; or
e. no decline in the rate of increase of levels of non-polymerized ASC and/or ASC specks between samples taken during treatment compared with samples taken before treatment are indicative of unsuccessful treatment of a disease, disorder and/or condition associated with non-polymerized ASC and/or ASC specks and thus the therapy is not selected for treatment.

In one embodiment, there is provided a method for assessing a candidate therapy for a disease, disorder and/or condition associated with non-polymerized ASC and/or ASC specks, the method comprising, following treatment of one or more subjects, contacting samples from the one or more treated subjects with an antibody or antigen-binding fragment of the invention, wherein lower levels of non-polymerized ASC and/or ASC specks in the samples compared with levels in corresponding samples from subjects not treated with the therapy are indicative of successful treatment of a disease, disorder and/or condition associated with non-polymerized ASC and/or ASC specks. The method may be performed multiple time points in matched samples between the treatment and placebo groups in order to monitor the effectiveness of the candidate therapy over a defined time period. The method may comprise contacting samples from the one or more treated subjects and the subjects not treated with the therapy with an antibody or antigen-binding fragment of the invention prior to treatment, with the therapy or placebo respectively, to determine base levels of for assessing a candidate therapy for a disease, disorder and/or condition associated with non-polymerized ASC and/or ASC specks.

In one embodiment, the ASC antibody or antigen-binding fragment thereof of the invention is for research use, in particular as an analytical tool or reference molecule.

### Definitions

ASC speck is a multiprotein inflammasome polymeric complex formed through homodimeric interactions between NLR's N-terminal pyrin and the ASC's N-terminal pyrin and the ASC's C-terminal CARD with the N-terminal CARD of pro-caspase-1 and further polymerized to form long helical filaments that are condensed into an intracellular macromolecular aggregate. This complex can be released into extracellular space and propagate inflammation.

Non-polymerized ASC includes monomeric ASC and oligomeric ASC. Non-polymerized ASC is predominantly diffused in the cytoplasm. A preferred form of non-polymerized ASC in the invention is monomeric ASC.

ASC polymerization is a process necessary for activation of ASC-dependent inflammasomes including NLRP3 inflammasome. ASC polymerization leads to formation of ASC speck complex, which induces the activation of pro-caspase-1 into active caspase that cleaves the inactive pro-IL-1β and pro-IL-18 forms into bioactive cytokines that activate downstream inflammatory pathways.

Propagation of inflammation caused by ASC speck spreading or cell-to-cell propagation of inflammation is a process in which ASC specks are released by inflammasome-activated cells into the extracellular space, where they continue to recruit and activate pro-caspase-1 and sustain IL-1β formation contributing thus to maintenance of inflammatory reaction. Extracellular ASC specks can be internalized by neighboring macrophages and seed endogenous ASC molecules in the cytosol of recipient cells resulting in IL-1 production by these cells.

ASC-dependent inflammasomes include NLRP1, NLRP2, NLRP3, NLRP6, NLRP7, NLRC4, NLRC5, NAIP2, NAIP5, NAIP6, HIN, AIM2, IFI-16, Pyrin and RIG-1.

The term “inhibition” is understood by one skilled in the art and can be measured with reference to a control in a relevant assay of the process to be inhibited, such as measurement of ASC polymerization, measurement of ASC dependent propagation of inflammation, and measurement of IL-1 release. Relevant inhibition may be 50%, 60%, 70% 80%, 90%, 95%, 96%, 97%, 98%, 99% or 100% (complete) relative to a specified control which does not result in any inhibition.

An “antigen binding molecule,” as used herein, is any molecule that can specifically or selectively bind to an antigen, in particular ASC. A binding molecule may include or be an antibody or a fragment thereof. An anti-ASC binding molecule is a molecule that binds to the ASC protein, such as an anti-ASC antibody or fragment thereof, at a specific recognition site, epitope. That is, antigen-binding molecules of the invention bind to an epitope within the amino acid sequence of SEQ ID NO: 1 and/or SEQ ID NO: 2. The antigen-binding molecules, in particular antibodies or antigen-binding fragments thereof, provided herein recognize full-length ASC. Other anti-ASC binding molecules may also include multivalent molecules, multi-specific molecules (e.g., diabodies), fusion molecules, aptamers, avimers, or other naturally occurring or recombinantly created molecules. Illustrative antigen-binding molecules useful in the present invention include antibody-like molecules. An antibody-like molecule is a molecule that can exhibit functions by binding to a target molecule (See, e.g., Current Opinion in Biotechnology 2006, 17:653-658; Current Opinion in Biotechnology 2007, 18:1-10; Current Opinion in Structural Biology 1997, 7:463-469; Protein Science 2006, 15:14-27), and includes, for example, DARPins (WO 2002/020565), Affibody (WO 1995/001937), Avimer (WO 2004/044011; WO 2005/040229), Adnectin (WO 2002/032925) and fynomers (WO 2013/135588).

The terms “anti ASC antibody” and “an antibody that binds to ASC” or simply “antibody” as used herein refer to an antibody that is capable of binding ASC with sufficient affinity such that the antibody is useful as a diagnostic and/or therapeutic agent in targeting ASC. In general, the term “antibody” is used herein in the broadest sense and encompasses various antibody structures, including but not limited to monoclonal antibodies, polyclonal antibodies, multispecific antibodies (e.g., bispecific or biparatopic antibodies), fully-human antibodies and antibody fragments so long as they exhibit the desired antigen-binding activity. Antibodies within the present invention may also be chimeric antibodies, recombinant antibodies, antigen-binding fragments of recombinant antibodies, humanized antibodies or antibodies displayed upon the surface of a phage or displayed upon the surface of a chimeric antigen receptor (CAR) T cell. An “antigen-binding fragment” of an antibody refers to a molecule other than an intact antibody that comprises a portion of an intact antibody and that binds the antigen to which the intact antibody binds.

Examples of antibody fragments include but are not limited to Fv, Fab, Fab′, Fab′-SH, F(ab′)2; intrabody; diabodies; linear antibodies; single-chain antibody molecules (e.g. scFv); and multispecific antibodies formed from antibody fragments.

An “antibody that binds to an epitope” within a defined region of a protein is an antibody that requires the presence of one or more of the amino acids within that region for binding to the protein.

In certain embodiments, an “antibody that binds to an epitope” within a defined region of a protein is identified by mutation analysis, in which amino acids of the protein are mutated, and binding of the antibody to the resulting altered protein (e.g., an altered protein comprising the epitope) is determined to be at least 20% of the binding to unaltered protein. In some embodiments, an “antibody that binds to an epitope” within a defined region of a protein is identified by mutation analysis, in which amino acids of the protein are mutated, and binding of the antibody to the resulting altered protein (e.g., an altered protein comprising the epitope) is determined to be at least 30%, at least 40%, at least 50%, at least 60%, at least 70%, at least 80%, or at least 90% of the binding to unaltered protein. In certain embodiments, binding of the antibody is determined by FACS, WB or by a suitable binding assay such as ELISA.

The term “binding to” as used in the context of the present invention defines a binding (interaction) of at least two “antigen-interaction-sites” with each other. The term “antigen-interaction-site” defines, in accordance with the present invention, a motif of a polypeptide, i.e., a part of the antibody or antigen-binding fragment of the present invention, which shows the capacity of specific interaction with a specific antigen or a specific group of antigens of ASC. Said binding/interaction is also understood to define a “specific recognition”. The term “specifically recognizing” means in accordance with this invention that the antibody is capable of specifically interacting with and/or binding to at least two amino acids of ASC SEQ ID NO: 1 (human ASC) and/or SEQ ID NO: 2 (mouse ASC).

The term “specific interaction” as used in accordance with the present invention means that the antibody or antigen-binding fragment thereof of the invention substantially does not cross-react with (poly)peptides of similar structures. Accordingly, the antibody or antigen-binding fragment thereof of the invention specifically binds to/interacts with structures of ASC formed by particular amino acid sequences within amino acids residues of SEQ ID NO: 1 and/or SEQ ID NO: 2.

SEQ ID No: 1 comprises the amino acid sequence
corresponding to human ASC (NP_037390.2 (Search:
NP_037390.2-NLM (nih.gov))):
(SEQ ID NO: 1)
MGRARDAILDALENLTAEELKKFKLKLLSVPLREGYGRIPRGALL
SMDALDLTDKLVSFYLETYGAELTANVLRDMGLQEMAGQLQAATH
QGSGAAPAGIQAPPQSAAKPGLHFIDQHRAALIARVTNVEWLLDA
LYGKVLTDEQYQAVRAEPTNPSKMRKLFSFTPAWNWTCKDLLLQA
LRESQSYLVEDLERS
SEQ ID No: 2 comprises the amino acid sequence
corresponding to mouse ASC (Search: NP_075747.3-
NLM (nih.gov))):
(SEQ ID NO: 2)
MGRARDAILDALENLSGDELKKFKMKLLTVQLREGYGRIPRGALL
QMDAIDLTDKLVSYYLESYGLELTMTVLRDMGLQELAEQLQTTKE
ESGAVAAAASVPAQSTARTGHFVDQHRQALIARVTEVDGVLDALH
GSVLTEGQYQAVRAETTSQDKMRKLFSFVPSWNLTCKDSLLQALK
EIHPYLVMDLEQS

Cross-reactivity of antigen-binding molecules, in particular a panel of antibodies or antigen-binding fragments thereof under investigation may be tested, for example, by assessing binding of said panel of antibodies or antigen-binding fragments thereof under conventional conditions (see, e.g., Harlow and Lane, Antibodies: A Laboratory Manual, Cold Spring Harbor Laboratory Press, (1988) and Using Antibodies: A Laboratory Manual, Cold Spring Harbor Laboratory Press, (1999)) to the (poly)peptide of interest as well as to a number of more or less (structurally and/or functionally) closely related (poly)peptides. Only those constructs (i.e. antibodies, antigen-binding fragments thereof and the like) that bind to the certain structure of ASC, e.g., a specific epitope or (poly)peptide/protein of ASC but do not or do not essentially bind to any of the other epitope or (poly)peptides of the same ASC, are considered specific for the epitope or (poly)peptide/protein of interest and selected for further studies in accordance with the method provided herein. These methods may comprise, inter alia, binding studies, blocking and competition studies with structurally and/or functionally closely related molecules. These binding studies also comprise FACS analysis, surface plasmon resonance (SPR, e.g. with BIACORE™), analytical ultracentrifugation, isothermal titration calorimetry, fluorescence anisotropy, fluorescence spectroscopy or by radiolabeled ligand binding assays.

Accordingly, specificity can be determined experimentally by methods known in the art and methods as described herein. Such methods comprise, but are not limited to Western Blots, ELISA-, RIA-, ECL-, IRMA-tests and peptide scans.

The term “KD” as used in accordance with the present invention refers to the equilibrium dissociation constant (also referred herein as the dissociation constant or the affinity constant) measuring the strength of a two-molecule interaction.

The term “ka” as used in accordance with the present invention refers to the association rate constant measuring the rate at which a complex is formed.

The term “kd” as used in accordance with the present invention refers to the dissociation rate measuring the rate of breakdown of a complex.

The term “monoclonal antibody” as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Monoclonal antibodies are advantageous in that they may be synthesized by a hybridoma culture, essentially uncontaminated by other immunoglobulins. The modified “monoclonal” indicates the character of the antibody as being amongst a substantially homogeneous population of antibodies, and is not to be construed as requiring production of the antibody by any particular method. As mentioned above, the monoclonal antibodies to be used in accordance with the present invention may be made by the hybridoma method described by Kohler, Nature 256 (1975), 495.

The term “polyclonal antibody” as used herein, refers to an antibody which was produced among or in the presence of one or more other, non-identical antibodies. In general, polyclonal antibodies are produced from a B-lymphocyte in the presence of several other B-lymphocytes which produced non-identical antibodies. Usually, polyclonal antibodies are obtained directly from an immunized animal.

The term “fully-human antibody” as used herein refers to an antibody which comprises human immunoglobulin protein sequences only. A fully human antibody may contain murine carbohydrate chains if produced in a mouse, in a mouse cell or in a hybridoma derived from a mouse cell. Similarly, “mouse antibody” or “murine antibody” refers to an antibody which comprises mouse/murine immunoglobulin protein sequences only. Alternatively, a “fully-human antibody” may contain rat carbohydrate chains if produced in a rat, in a rat cell, in a hybridoma derived from a rat cell. Similarly, the term “rat antibody” refers to an antibody that comprises rat immunoglobulin sequences only. Fully-human antibodies may also be produced, for example, by phage display which is a widely used screening technology which enables production and screening of fully human antibodies. Also phage antibodies can be used in context of this invention. Phage display methods are described, for example, in U.S. Pat. Nos. 5,403,484, 5,969,108 and 5,885,793. Another technology which enables development of fully-human antibodies involves a modification of mouse hybridoma technology. Mice are made transgenic to contain the human immunoglobulin locus in exchange for their own mouse genes (see, for example, U.S. Pat. No. 5,877,397).

The term “chimeric antibodies”, refers to an antibody which comprises a variable region of the present invention fused or chimerized with an antibody region (e.g., constant region) from another, human or non-human species (e.g., mouse, horse, rabbit, dog, cow, chicken).

The term antibody also relates to recombinant human antibodies, heterologous antibodies and heterohybrid antibodies. The term “recombinant (human) antibody” includes all human sequence antibodies that are prepared, expressed, created or isolated by recombinant means, such as antibodies isolated from an animal (e.g., a mouse) that is transgenic for human immunoglobulin genes; antibodies expressed using a recombinant expression vector transfected into a host cell, antibodies isolated from a recombinant, combinatorial human antibody library, or antibodies prepared, expressed, created or isolated by any other means that involves splicing of human immunoglobulin gene sequences to other DNA sequences. Such recombinant human antibodies have variable and constant regions (if present) derived from human germline immunoglobulin sequences. Such antibodies can, however, be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

A “heterologous antibody” is defined in relation to the transgenic non-human organism producing such an antibody. This term refers to an antibody having an amino acid sequence or an encoding nucleic acid sequence corresponding to that found in an organism not consisting of the transgenic non-human animal, and generally from a species other than that of the transgenic non-human animal.

The term “heterohybrid antibody” refers to an antibody having light and heavy chains of different organismal origins. For example, an antibody having a human heavy chain associated with a murine light chain is a heterohybrid antibody. Examples of heterohybrid antibodies include chimeric and humanized antibodies.

The term antibody also relates to humanized antibodies. “Humanized” forms of non-human (e.g. murine or rabbit) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Often, humanized antibodies are human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Furthermore, humanized antibody may comprise residues, which are found neither in the recipient antibody nor in the imported CDR or framework sequences. These modifications are made to further refine and optimize antibody performance. In general, the humanized antibody will comprise substantially all of at least one, and typically two variable domains, in which all or substantially all of the CDR regions correspond to those of a non-human immunoglobulin and all or substantially all of the FR regions are those of a human immunoglobulin consensus sequence. The humanized antibody may also comprise at least a portion of an immunoglobulin constant region (Fc), typically that of a human immunoglobulin. For further details, see: Jones et al., Nature 321 (1986), 522-525; Reichmann Nature 332 (1998), 323-327 and Presta Curr Op Struct Biol 2 (1992), 593-596.

A popular method for humanization of antibodies involves CDR grafting, where a functional antigen-binding site from a non-human ‘donor’ antibody is grafted onto a human ‘acceptor’ antibody. CDR grafting methods are known in the art and described, for example, in U.S. Pat. Nos. 5,225,539, 5,693,761 and 6,407,213. Another related method is the production of humanized antibodies from transgenic animals that are genetically engineered to contain one or more humanized immunoglobulin loci which are capable of undergoing gene rearrangement and gene conversion (see, for example, U.S. Pat. No. 7,129,084).

Accordingly, in context of the present invention, the term “antibody” relates to full immunoglobulin molecules as well as to parts of such immunoglobulin molecules (i.e., “antigen-binding fragment thereof”). Furthermore, the term relates, as discussed above, to modified and/or altered antibody molecules. The term also relates to recombinantly or synthetically generated/synthesized antibodies. The term also relates to intact antibodies as well as to antibody fragments thereof, like, separated light and heavy chains, Fab, Fv, Fab′, Fab′-SH, F(ab′)2. The term antibody also comprises but is not limited to fully-human antibodies, chimeric antibodies, humanized antibodies, CDR-grafted antibodies and antibody constructs, like single chain Fvs (scFv) or antibody-fusion proteins. “Single-chain Fv” or “scFv” antibody fragments have, in the context of the invention, the V_{H} and V_{L} domains of an antibody, wherein these domains are present in a single polypeptide chain. Generally, the scFv polypeptide further comprises a polypeptide linker between the V_{H} and V_{L} domains which enables the scFv to form the desired structure for antigen binding. Techniques described for the production of single chain antibodies are described, e.g., in Pluckthun in The Pharmacology of Monoclonal Antibodies, Rosenburg and Moore eds. Springer-Verlag, N.Y. (1994), 269-315.

A “Fab fragment” as used herein is comprised of one light chain and the C_{H}1 and variable regions of one heavy chain. The heavy chain of a Fab molecule cannot form a disulfide bond with another heavy chain molecule.

An “Fc” region contains two heavy chain fragments comprising the C_{H}2 and C_{H}3 domains of an antibody.

The two heavy chain fragments are held together by two or more disulfide bonds and by hydrophobic interactions of the C_{H}3 domains.

A “Fab′ fragment” contains one light chain and a portion of one heavy chain that contains the V_{H} domain and the C_{H}1 domain and also the region between the C_{H}1 and C_{H}2 domains, such that an interchain disulfide bond can be formed between the two heavy chains of two Fab′ fragments to form a F(ab′)₂ molecule.

A “F(ab′)₂ fragment” contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains.

The “Fv region” comprises the variable regions from both the heavy and light chains, but lacks the constant regions.

Antibodies, antibody constructs, antibody fragments, antibody derivatives (all being Ig-derived) to be employed in accordance with the invention or their corresponding immunoglobulin chain(s) can be further modified using conventional techniques known in the art, for example, by using amino acid deletion(s), insertion(s), substitution(s), addition(s), and/or recombination(s) and/or any other modification(s) known in the art either alone or in combination. Methods for introducing such modifications in the DNA sequence underlying the amino acid sequence of an immunoglobulin chain are well known to the person skilled in the art; see, e.g., Sambrook et al., Molecular Cloning: A Laboratory Manual; Cold Spring Harbor Laboratory Press, 2^{nd} edition (1989) and 3^{rd} edition (2001). The term “Ig-derived domain” particularly relates to (poly)peptide constructs comprising at least one CDR. Fragments or derivatives of the recited Ig-derived domains define (poly)peptides which are parts of the above antibody molecules and/or which are modified by chemical/biochemical or molecular biological methods. Corresponding methods are known in the art and described inter alia in laboratory manuals (see Sambrook et al., Molecular Cloning: A Laboratory Manual; Cold Spring Harbor Laboratory Press, 2^{nd} edition (1989) and 3^{rd} edition (2001); Gerhardt et al., Methods for General and Molecular Bacteriology ASM Press (1994); Lefkovits, Immunology Methods Manual: The Comprehensive Sourcebook of Techniques; Academic Press (1997); Golemis, Protein-Protein Interactions: A Molecular Cloning Manual Cold Spring Harbor Laboratory Press (2002)).

The term “CDR” as employed herein relates to “complementary determining region”, which is well known in the art. The CDRs are parts of immunoglobulins that determine the specificity of said molecules and make contact with a specific ligand. The CDRs are the most variable part of the molecule and contribute to the diversity of these molecules. There are three CDR regions CDR1, CDR2 and CDR3 in each V domain. CDR-H depicts a CDR region of a variable heavy chain and CDR-L relates to a CDR region of a variable light chain. VH means the variable heavy chain and VL means the variable light chain. The CDR regions of an Ig-derived region may be determined as described in Kabat “Sequences of Proteins of Immunological Interest”, 5^{th} edit. NIH Publication no. 91-3242 U.S. Department of Health and Human Services (1991). CDR sequences provided herein are defined according to Kabat. However, it will be understood by the skilled person that the invention is intended to encompass binding molecules in which the CDR sequences are defined according to any useful identification/numbering scheme. For example, Chothia (Canonical structures for the hypervariable regions of immunoglobulins. Chothia C, Lesk A M. J Mol Biol. 1987 Aug. 20; 196(4):901-17), IMGT (IMGT, the international ImMunoGeneTics database. Giudicelli V_{H}, Chaume D, Bodmer J, Muller W, Busin C, Marsh S, Bontrop R, Marc L, Malik A, Lefranc M P. Nucleic Acids Res. 1997 Jan. 1; 25(1):206-11 and Unique database numbering system for immunogenetic analysis. Lefranc MP. Immunol Today. 1997 November; 18(11):509), MacCallum (MacCallum R M, Martin A C, Thornton J M, J Mol Biol. 1996 Oct. 11; 262(5):732-45) and Martin (Abhinandan K R, Martin A C R. Analysis and improvements to Kabat and structurally correct numbering of antibody variable domains. Mol Immunol. (2008) 45:3832-9. 10.1016/j.molimm.2008.05.022) numbering schemes may be adopted in order to define the CDRs.

Accordingly, in the context of the present invention, the antibody molecule described herein above is selected from the group consisting of a full antibody (immunoglobulin, like an IgG1, an IgG2, an IgG2a, an IgG2b, an IgA1, an IgGA2, an IgG3, an IgG4, an IgA, an IgM, an IgD or an IgE), F(ab)-, Fab′-SH-, Fv-, Fab′-, F(ab′)2—fragment, a chimeric antibody, a CDR-grafted antibody, a fully human antibody, a bivalent antibody-construct, an antibody-fusion protein, a synthetic antibody, bivalent single chain antibody, a trivalent single chain antibody and a multivalent single chain antibody. “Humanization approaches” are well known in the art and in particular described for antibody molecules, e.g. Ig-derived molecules. The term “humanized” refers to humanized forms of non-human (e.g., murine) antibodies or fragments thereof (such as Fv, Fab, Fab′, F(ab′), scFvs, or other antigen-binding partial sequences of antibodies) which contain some portion of the sequence derived from non-human antibody. Humanized antibodies include human immunoglobulins in which residues from a complementary determining region (CDR) of the human immunoglobulin are replaced by residues from a CDR of a non-human species such as mouse, rat or rabbit having the desired binding specificity, affinity and capacity. In general, the humanized antibody will comprise substantially all of at least one, and generally two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin; see, inter alia, Jones et al., Nature 321 (1986), 522-525, Presta, Curr. Op. Struct. Biol. 2 (1992), 593-596. Methods for humanizing non-human antibodies are well known in the art. Generally, a humanized antibody has one or more amino acids introduced into it from a source which is non-human still retain the original binding activity of the antibody. Methods for humanization of antibodies/antibody molecules are further detailed in Jones et al., Nature 321 (1986), 522-525; Reichmann et al., Nature 332 (1988), 323-327; and Verhoeyen et al., Science 239 (1988), 1534-1536. Specific examples of humanized antibodies, e.g. antibodies directed against EpCAM, are known in the art (see e.g. LoBuglio, Proceedings of the American Society of Clinical Oncology Abstract (1997), 1562 and Khor, Proceedings of the American Society of Clinical Oncology Abstract (1997), 847).

Accordingly, in the context of this invention, antibody molecules or antigen-binding fragments thereof are provided, which are humanized and can successfully be employed in pharmaceutical compositions. The specificity of the antibody or antigen-binding fragment of the present invention may not only be expressed by the nature of the amino acid sequence of the antibody or the antigen-binding fragment as defined above but also by the epitope to which the antibody is capable of binding.

It may be understood by a person skilled in the art that the epitopes may be comprised in the ASC protein, but may also be comprised in a degradation product thereof or may be a chemically synthesized peptide. The amino acid positions are only indicated to demonstrate the position of the corresponding amino acid sequence in the sequence of the ASC protein. The invention encompasses all peptides comprising the epitope. The peptide may be a part of a polypeptide of more than 100 amino acids in length or may be a small peptide of less than 100, preferably less than 50, more preferably less than 25 amino acids, even more preferably less than 16 amino acids. The amino acids of such peptide may be natural amino acids or nonnatural amino acids (e.g., beta-amino acids, gamma-amino acids, D-amino acids) or a combination thereof. Further, the present invention may encompass the respective retro-inverso peptides of the epitopes.

The peptide may be unbound or bound. It may be bound, e.g., to a small molecule (e.g., a drug or a fluorophore), to a high-molecular weight polymer (e.g., polyethylene glycol (PEG), polyethylene imine (PEI), hydroxypropylmethacrylate (HPMA), etc.) or to a protein, a fatty acid, a sugar moiety or may be inserted in a membrane.

In order to test whether an antibody in question and the antibody of the present invention recognize the same epitope, the following competition study may be carried out: Vero cells infected with 3 MOI (multiplicity of infection) are incubated after 20 h with varying concentrations of the antibody in question as the competitor for 1 hour. In a second incubation step, the antibody of the present invention is applied in a constant concentration of 100 nM and its binding is flow-cytometrically detected using a fluorescence-labelled antibody directed against the constant domains of the antibody of the invention. Binding that conducts anti-proportional (inversely proportional) to the concentration of the antibody in question is indicative that both antibodies recognize the same epitope. However, many other assays are known in the art which may be used.

The present invention also relates to the production of specific antibodies against native polypeptides and recombinant polypeptides of ASC. This production is based, for example, on the immunization of animals, like mice. However, also other animals for the production of antibody/antisera are envisaged within the present invention. For example, monoclonal and polyclonal antibodies can be produced by rabbit, mice, goats, donkeys and the like. The polynucleotide encoding a correspondingly chosen polypeptide of ASC can be subcloned into an appropriate vector, wherein the recombinant polypeptide is to be expressed in an organism capable of expression, for example in bacteria. Thus, the expressed recombinant protein can be intra-peritoneally injected into a mice and the resulting specific antibody can be, for example, obtained from the mice serum being provided by intra-cardiac blood puncture. The present invention also envisages the production of specific antibodies against native polypeptides and recombinant polypeptides by using a DNA vaccine strategy as exemplified in the appended examples. DNA vaccine strategies are well-known in the art and encompass liposome-mediated delivery, by gene gun or jet injection and intramuscular or intradermal injection. Thus, antibodies directed against a polypeptide or a protein or an epitope of ASC, in particular the epitope of the antibodies provided herein, can be obtained by directly immunizing the animal by directly injecting intramuscularly the vector expressing the desired polypeptide or aprotein or an epitope of ASC which lies within SEQ ID NO:1 and/or SEQ ID NO: 2. The amount of obtained specific antibody can be quantified using an ELISA, which is also described herein below. Further methods for the production of antibodies are well known in the art, see, e.g. Harlow and Lane, “Antibodies, A Laboratory Manual”, CSH Press, Cold Spring Harbor, 1988.

Thus, under designated assay conditions, the specified antibodies and the corresponding epitope of ASC bind to one another and do not bind in a significant amount to other components present in a sample. Specific binding to a target analyte under such conditions may require a binding moiety that is selected for its specificity for a particular target analyte. A variety of immunoassay formats may be used to select antibodies specifically reactive with a particular antigen. For example, solid-phase ELISA immunoassays are routinely used to select monoclonal antibodies specifically immunoreactive with an analyte. See Shepherd and Dean (2000), Monoclonal Antibodies: A Practical Approach, Oxford University Press and/or Howard and Bethell, for a description of immunoassay formats and conditions that can be used to determine specific immunoreactivity. Typically a specific or selective reaction will be at least twice background signal to noise and more typically more than 10 to 100 times greater than background. The person skilled in the art is in a position to provide for and generate specific binding molecules directed against the novel polypeptides. For specific binding-assays it can be readily employed to avoid undesired cross-reactivity, for example polyclonal antibodies can easily be purified and selected by known methods (see Shepherd and Dean, loc. Cit.).

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG2a, IgG2b, IgG3, IgG4, IgA1, and IgA2.

The heavy chain constant domains that correspond to the different classes of immunoglobulins are called α, δ, ε, γ, and μ, respectively.

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In certain embodiments, antibody variants having one or more amino acid substitutions are provided. Sites of interest for substitutional mutagenesis include the CDRs and FRs. Conservative substitutions are shown in Table 0 under the heading of “preferred substitutions.” More substantial changes are provided in Table 0 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 0

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gin |
| Asp (D) | Glu; Asn | Glu |
| Cys (C) | Ser; Ala | Ser |
| Gin (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class. One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more CDR residues are mutated and the variant antibodies displayed on phage and screened for a particular biological activity (e.g. binding affinity).

Alterations (e.g., substitutions) may be made in CDRs, e.g., to improve antibody affinity. Such alterations may be made in CDR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al., in Methods in Molecular Biology 178:1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR H3 and CDR-L3 in particular are often targeted.

In certain embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. Such alterations may be outside of CDR “hotspots” or SDRs. In certain embodiments of the variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244: 1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex is used to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al., TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; see Edelman, G. M. et al., Proc. Natl. Acad. USA, 63, 78-85 (1969)); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al., J. Mol. Biol. 336:1239-1249 (2004); Yamane-Ohnuki et al., Biotech. Bioeng. 87: 614 (2004). Examples of cell lines capable of producing defucosylated antibodies include Lec13 CHO cells deficient in protein fucosylation (Ripka et al., Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al., Bioteeh. Bioeng. 87: 614 (2004); Kanda, Y. et al., Bioteehnol. Bioeng., 94(4):680-688 (2006); and WO2003/085 107).

Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the invention contemplates an antibody variant that possesses some but not all effector functions, which make it a desirable candidate for applications in which the half life of the antibody in vivo is important yet certain effector functions (such as complement activation and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes and microglia express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al., Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)).

Alternatively, non-radioactive assays methods may be employed (see, for example, ACTI™ non radioactive cytotoxicity assay for flow cytometry (CellTechnology, Inc. Mountain View, CA; and CytoTox 96@non-radioactive cytotoxicity assay (Promega, Madison, WI). Useful effector cells for such assays include peripheral blood mononuclear cells (PBMC) and Natural Killer (NK) cells.

Alternatively, or additionally, ADCC activity of the molecule of interest may be assessed in vivo, e.g., in a animal model such as that disclosed in Clynes et al., Proc. Nat'l Acad. sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581). Alternatively, antibodies with reduced effector function include those with substitution of one or more of Fc region residues 234, 235 and 329, so-called “PG-LALA” Fc mutant with substitution of residues 234 and 235 to alanine and 329 to glycine (Lo, M. et al., Journal of Biochemistry, 292, 3900-3908).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001)).

In certain embodiments, an antibody variant comprises a Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al., J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kirn et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40 (1988); U.S. Pat. Nos. 5,648,260; 5,624,821; and WO 94/29351 concerning other examples of Fc region variants.

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

In certain embodiments, an antibody provided herein may be further modified to contain additional nonproteinaceous moieties that are known in the art and readily available. The moieties suitable for derivatization of the antibody include but are not limited to water soluble polymers. Nonlimiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

Antibodies may be produced using recombinant methods and compositions, e.g., as described in U.S. Pat. No. 4,816,567. In one embodiment, isolated nucleic acid encoding an anti-ASC antibody described herein is provided. Such nucleic acid may encode an amino acid sequence comprising the VL and/or an amino acid sequence comprising the VH of the antibody (e.g., the Light and/or Heavy Chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., YO, NSO, Sp20). In one embodiment, a method of making an anti-ASC antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-ASC antibody, nucleic acid encoding an antibody, e.g., as described above, is isolated and inserted into one or more vectors for further cloning and/or expression in a host cell or a cell-free expression system. Such nucleic acid may be readily isolated and sequenced using conventional procedures (e.g., by using oligonucleotide probes that are capable of binding specifically to genes encoding the Heavy and Light Chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Val. 248 (B. K. C. Lo, ed., Humana Press, Totowa, NJ, 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are macaque kidney CVl line transformed by SV40 (COS-7); human embryonic kidney line (293 or 293 cells as described, e.g., in Graham et al., J. Gen Viral. 36:59 (1977)); baby hamster kidney cells (BHK); mouse sertoli cells (TM4 cells as described, e.g., in Mather, Biol. Reprod. 23:243-251 (1980)); macaque kidney cells (CV 1); African green macaque kidney cells (VERO-76); human cervical carcinoma cells (HeLa); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N. Y Aead. Sei. 383:44-68 (1982); MRC 5 cells; and FS4 cells. Other useful mammalian host cell lines include Chinese hamster ovary (CHO) cells, including DHFR CHO cells (Urlaub et al., Proc. Natl. Acad. cii. USA 77:4216 (1980)); and myeloma cell lines such as YO, NSO and Sp2/0. For a review of certain mammalian host cell lines suitable for antibody production, see, e.g., Yazaki and Wu, Methods in Molecular Biology, Val. 248 (B. K. C. Lo, ed., Humana Press, Totowa, NJ), pp. 255-268 (2003).

Methods for producing an anti-ASC antibody or an antigen-binding fragment thereof of the invention, in particular an antibody, may comprise the steps of:
a. culturing a suitable host cell or cell-free expression system under conditions suitable for producing the binding molecule, in particular the antibody; and
b. isolating the binding molecule, in particular the antibody. Suitable culturing and isolation techniques are available to the skilled person.

Anti-ASC antibodies provided herein may be identified, screened for, or characterized for their physical/chemical properties and/or biological activities by various assays known in the art.

In one aspect, an antibody of the invention is tested for its antigen binding activity, e.g., by known methods such as ELISA, BIACore®, FACS, immunofluorescence or immunohistochemistry.

In another aspect, competition assays may be used to identify an antibody that competes with any of the antibodies described herein for binding to ASC. In certain embodiments, such a competing antibody binds to the same epitope (e.g., a linear or a conformational epitope) that is bound by an antibody described herein. Detailed exemplary methods for mapping an epitope to which an antibody binds are provided in Morris (1996) “Epitope Mapping Protocols,” in Methods in Molecular Biology vol. 66 (Humana Press, Totowa, NJ).

In an exemplary competition assay, immobilized ASC is incubated in a solution comprising a first labeled antibody that binds to ASC (e.g., any of the antibodies described herein) and a second unlabeled antibody that is being tested for its ability to compete with the first antibody for binding to ASC. As a control, immobilized ASC is incubated in a solution comprising the first labeled antibody but not the second unlabeled antibody. After incubation under conditions permissive for binding of the first antibody to ASC, excess unbound antibody is removed, and the amount of label associated with immobilized ASC is measured. If the amount of label associated with immobilized ASC is substantially reduced in the test sample relative to the control sample, then that indicates that the second antibody is competing with the first antibody for binding to ASC. See Harlow and Lane (1988) Antibodies: A Laboratory Manual ch.14 (Cold Spring Harbor Laboratory, Cold Spring Harbor, NY).

The invention also provides immunoconjugates comprising an anti-ASC antibody provided herein conjugated to one or more therapeutic agents, such as chemotherapeutic agents or drugs, growth inhibitory agents, toxins (e.g., protein toxins, enzymatically active toxins of bacterial, fungal, plant, or animal origin, or fragments thereof), radioactive isotopes (i.e., a radioconjugate), blood brain barrier penetration moieties or detectable labels.

In another aspect of the invention, an article of manufacture containing materials useful for prevention, alleviation, treatment and/or diagnosis of diseases, disorders and abnormalities associated with accumulation of extracellular ASC specks described above is provided. The article of manufacture comprises a container and a label or package insert on or associated with the container. Suitable containers include, for example, bottles, vials, syringes, intravenous (IV) solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises a further therapeutic agent. The article of manufacture in this embodiment of the invention may further comprise a package insert indicating that the compositions can be used to treat a particular condition.

Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

It is understood that any of the above articles of manufacture may include an immunoconjugate of the invention in place of or in addition to an anti-ASC antibody.

### DESCRIPTION OF FIGURES

FIG. 1: Analysis of human and mouse ASC target engagement for ASC mAbs by immunoblot. ASC mAbs were analyzed by WB on a human monocytic cell line (H. Monoc) and mouse macrophage cell lysates (J774.A1) using recombinant human (Rec H) and mouse (Rec M) ASC protein (MBP-tagged or N-terminal 10xHis-tagged and C-terminal Myc-tagged) as positive control and a human monocytic ASC KO cell lysate (H. ASC KO) as a negative control. The respective mAbs used for detection are indicated at the bottom of each immunoblot. Molecular weight marker is shown on the left, kDa, Kilodalton. Arrows indicate the expected molecular weight for ASC and recombinant ASC proteins.

FIG. 2: Human ASC target engagement with ASC mAbs. Representative images showing cytoplasmic ASC and ASC specks labeled by selected ASC mAbs analyzed by immunofluorescence in human monocytes. Arrows indicate ASC specks. Inset shows higher magnification images.

FIG. 3: Representative polymerization kinetics of human ASC in the presence of degressive amount of mAbs. Each plot shows polymerization kinetic in the presence of serial dilutions of tested mAb (concentration range from 200 nM to 0.09 nM with dilution 1/3). Each antibody was tested in n=2 or n=3 experiments. FA—fluorescence anisotropy.

FIG. 4. Assessment of antibody-driven uptake of ASC polymers. Representative images of pHrodo fluorescence in human monocytic cells treated with human ASC polymers for 3h in the presence of mAbs. mAb name indicated on top of each image. Graph shows kinetics of fluorescent signal monitored every hour for 22 h. Pol—hASC polymers.

FIG. 5. Inhibition of IL-1p release by ASC mAbs in a human monocytic cell line treated with hASC polymers. Human monocytes differentiated into macrophages were primed and treated with hASC polymers preincubated with anti-ASC mAbs at different concentrations or with IgG2a isotype control at 420 nM or with ACI-8016-401H9B7-AB1 (internal reference) at 42 nM. Levels of IL-1β were determined by AlphaLISA and expressed as percent of control with 0% and 100% corresponding to buffer and hASC polymers incubated with isotype control mAbs respectively. Representative results for ACI-8016-401H9B7-AB1 (A), ACI-8016-18F4C12-AB1 (B) and ACI-8016-31F10C5-AB1 are shown. IC50 values (nM) were retrieved from nonlinear regression (curve fit) using GraphPad software. Pol—hASC polymers, ref—ACI-8016-401H9B7-AB1. Data for each concentration are shown as mean±SD.

FIG. 6. Epitope mapping of ASC mAbs. The binding of ASC antibodies to alanine mutants of PYD domain of PYCARD was measured by ELISA. Alanine mutants were captured using anti-MBP antibody, and binding of ASC antibodies was detected with an anti-mouse IgG2a-HRP antibody. The binding response was normalized to the hPYD-WT for each antibody. Mutations showing at least 70% of reduction in binding define the key/critical binding residues of each antibody.

FIG. 7. Epitope mapping of ASC mAbs specific for the CARD domain. The binding of CARD antibodies to alanine mutants of PYCARD are measured by ELISA. Alanine mutants are captured using anti-MBP antibody, and binding of CARD antibodies was detected with an anti-mouse IgG2a-HRP antibody. The binding response is normalized to the human PYCARD -WT for each antibody. Mutations showing at least 70% of reduction in binding define the key/critical binding, residue of each antibody.

FIG. 8. The effect of ASC mAbs on DMNI clinical score progression and spleen size. A) Clinical course of MOG35-55-induced DMNI in C57BL/6 mice treated with ASC-targeting mAbs (ACI-8016-18F4C12-AB1 and ACI-8016-32B6C7-AB1) or IgG2a isotype control mAb injected at 30 mg/kg i.p. on days 8, 11, and 13 post-immunization. Results are expressed as daily mean clinical score ±SEM of 12 mice/group. B) Mean Maximum score (MMS), considered as highest clinical score reached by a mouse within the study paradigm. Results are expressed as MMS ±SD of 12 mice/group; *p<0.05, One-Way ANOVA, Dunnett's multiple comparison test. C) Spleen mass normalized to body weight of 12 mice/group on day 17 post-immunization. * p<0.05, One-Way ANOVA, Dunnett's multiple comparison test (B and C), n.s.—not significant.

FIG. 9. The effect of ASC mAbs on DMNI pathology. A) Demyelination score presented as loss of MBP staining in the spinal cord of mice treated with ACI-8016-32B6C7-AB1 or IgG2a isotype control mAb. Results are expressed as demyelination score ±SEM of 12 mice/group. B) CD4 and C) Iba1 immunoreactive (IR) area in the spinal cord of mice treated with ACI-8016-32B6C7-AB1 or IgG2a isotype control mAb. Results are expressed as IR area of total tissue area (%) ±SEM of 12 mice/group. * p<0.05, **p<0.01, Student's t-test.

FIG. 10. The effect of ASC mAbs on inflammasome-related proteins. ASC (A) and cleaved caspase-1 (B) spinal cord (thoracic-lumbar section) protein expression calculated as fold change of IgG2a isotype control group. Values correspond to corrected area (total area normalized to protein concentration as assessed by JESS software). Results are expressed as arbitrary unit (A.U.) ±SEM of 12 mice/group. *p<0.05, unpaired t-test.

The invention will be further understood with reference to the following non-limiting examples: EXAMPLES.

### Example 1: Antibody Generation

### A. Mouse Immunization

C57BL/6JOlaHsd (C57BL/6) wild-type mice (Harlan, USa) and C57BL/6NTac-Pycardem6711Tac (ASC KO animals generated by Taconic) mice were injected subcutaneously (s.c) with 200 μL of vaccine. Vaccinations started at 10 weeks. Mice were vaccinated with full-length ASC protein presented on the surface of liposomes in the presence of Monophosphoryl Hexa-acyl Lipid A, 3-Deacyl (Synthetic) (3D-(6-acyl) PHAD®) (Avanti Polar Lipids, USA) as adjuvant.

Animals received four s.c. injections at days 0, 17, 31 and 59. Blood samples were collected 3 days before the first immunization (to serve as the baseline control) and at study days 24, 38 and 66. Prior to lymph node fusions, mice were immunized by s.c. injections three days and one day before fusions. Vaccine response was measured in mouse plasma. Binding of plasma derived antibodies from immunized mice to immobilized recombinant full-length (FL) ASC indicated high titers for antibodies against ASC.

### B. Generation of Hybridomas and Selection for Subcloning

Mice were euthanized and eight independent fusions were performed (4 per mouse) with myeloma X63/AG.8653. Resulting hybridoma-derived antibodies were screened for binding to human and mouse ASC protein by ELISA.

### Example 2: Antibodies Characterization by ELISA Methods

### Immunoassay Protocol (ELISA)

Proteins and peptides were diluted in PBS. Final concentration of 3 μg/mL for human and mouse ASC proteins and 2 μg/mL for PYD and CARD domain of hASC was used to coat ELISA plates (MaxiSorp, Nunc) overnight (ON) at 4° C., 50 μL/well. After washing four times with PBS/0.05% Tween-20 and blocking for one hour at 37° C. (PBS/0.05% Tween-20 (Merck, cat. n° 8.22184.0500)/1% BSa (Sigma, cat. n° A9418)), plates were incubated for two hours at 37° C. with mAbs using PBS/0.05% Tween-20/1% BSA as diluent. Dilution series of anti-human ASC clone B3 (SantaCruz BioTechnologies, cat. n° sc-514414), or anti-mouse ASC clone 2EI-7 IgG1 mAb (Merck Millipore, cat. n° 04-147), both starting at 1 μg/mL were used as positive controls, against human and mouse ASC respectively, where applicable. Next, plates were washed four times with PBS/0.05% Tween-20 and incubated for two hours at 37° C. with Goat anti-Mouse IgG-AP antibody (Jackson Immunoresearch, cat. n° 115-055-164) at 1/1000 dilution. After washing, plates were incubated two hours at RT with 1 mg/mL of phosphatase substrate pNPp (Sigma, cat. n° S0942). For EC50 determination, plates were coated with 2-3 μg/mL of protein or peptide and read at one hour. The absorbance signal was read at 405 nm wavelength using a plate reader (Tecan Infinity M200). EC50 was calculated using GraphPad Prism 8 and a non-constrain variable slope 4 parameter fit.

### Results

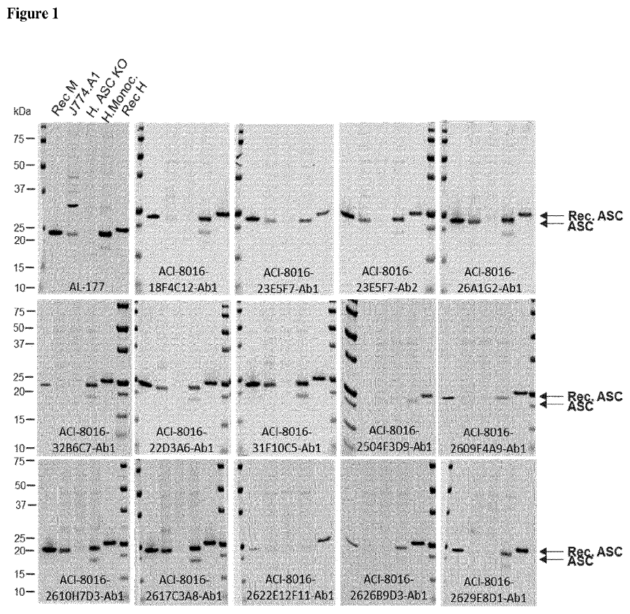

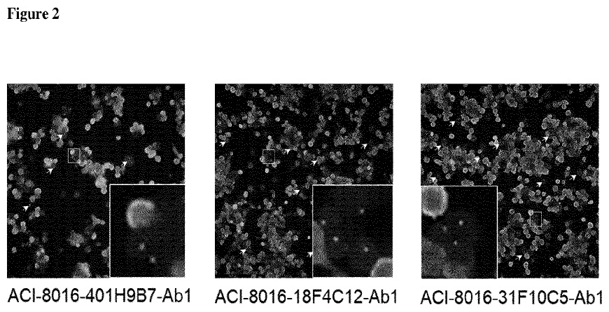

The binding potency of a mAb to human ASC (hASC), mouse ASC (mASC), human PYD (hPYD) and human CARD (hCARD) was determined by ELISA. Binding profiles derived from serial dilutions of each mAb are shown in FIG. 1. The commercially available anti-human ASC mAb clone B3 and anti-mouse ASC clone 2EI-7 served as positive control for human and mouse ASC binding in all ELISA experiments. All tested mAbs showed binding to human ASC and demonstrated EC50's for binding to the human ASC protein between 0.05 and 0.27 nM, summarized in Table 1. ACI-8016-416E6G4-AB1, ACI-8016-203B12C3-AB1, ACI-8019-2314F6H11-AB1, ACI-8016-18F4C12-AB1, ACI-8016-23E5F7-AB1, ACI-8016-23E5F7-AB2, ACI-8016-26A1G2-AB1, ACI-8016-22D3A6-AB1, ACI-8016-31F10C5-AB1, ACI-8016-3E6B11-AB1, ACI-8016-11A3F3-AB1, ACI-8016-14G5B8-AB1, ACI-8016-22A10F8-AB1, ACI-8016-27A1G4-AB1, ACI-8016-29C5E11-AB1, ACI-8016-7G3B5-AB1, ACI-8016-2504F3D9-AB1, ACI-8016-2516A8C6-AB1, ACI-8016-2602H6F10-AB1, ACI-8016-2609F4A9-AB1, ACI-8016-2610H7D3-AB1, ACI-8016-2614C3B2-AB1, ACI-8016-2617C3A8-AB1, ACI-8016-2622E12F11-AB1, ACI-8016-2626B9D3-AB1 and ACI-8016-2629E8D1-AB1 showed cross-reactivity to mouse ASC with EC50 in the range 0.07 and 4.52 nM. Clone binding to human PYD and CARD domain was determined by ELISA. EC50 values are summarized in Table 1.

TABLE 1
EC₅₀ values for binding of mAbs to hASC, mASCI, hPYD and hCARD

| | ELISA | ELISA | Human PYD | Human CARD |
|---|---|---|---|---|
| | hASC EC50 | mASC EC50 | domain | domain |
| Antibody name | [nM] | [nM] | [nM] | [nM] |
| ACI-8016-416E6G4-AB1 | 0.07 | 0.07 | 0.07 | no binding |
| ACI-8016-402H11C9-Ab1 | 0.06 | no binding | no binding | 0.06 |
| ACI-8016-203B12C3-AB1 | 0.10 | 0.30 | no binding | 0.067 |
| ACI-8016-421B10C12D2-AB1 | 0.06 | no binding | 0.07 | no binding |
| ACI-8016-417E12A8-AB1 | 0.07 | no binding | 0.06 | no binding |
| ACI-8016-413G10A5-AB1 | 0.05 | no binding | no binding | 0.06 |
| ACI-8016-407E10A9-AB1 | 0.06 | no binding | no binding | 0.07 |
| ACI-8016-203G8B10-AB1 | 0.09 | no binding | 0.08 | no binding |
| ACI-8016-401H9B7-AB1 | 0.10 | no binding | 0.06 | no binding |
| ACI-8016-1112B3D7-AB1* | 0.07 | no binding | NA | NA |
| ACI-8018-2221B7F1-AB1* | 0.14 | no binding | NA | NA |
| ACI-8019-2314F6H11-AB1 | 0.10 | 0.20 | N.D. | N.D. |
| ACI-8016-207E8B2-AB1* | 0.10 | 0.11 | 0.12 | no binding |
| ACI-8016-2A1B12-AB1* | 0.17 | 1.56 | 3.34 | 0.13 |
| ACI-8016-17H1G2-AB1* | 0.13 | 0.19 | no binding | 0.10 |
| ACI-8016-18F4C12-AB1 | 0.19 | 0.20 | no binding | 0.18 |
| ACI-8016-23E5F7-AB1 | 0.12 | 0.15 | 0.13 | no binding |
| ACI-8016-23E5F7-AB2 | 0.09 | 0.15 | 0.14 | no binding |
| ACI-8016-26A1G2-AB1 | 0.13 | 0.15 | 0.12 | no binding |
| ACI-8016-32B6C7-AB1 | 0.14 | 0.14 | no binding | 0.11 |
| ACI-8016-22D3A6-AB1 | 0.27 | 0.27 | 0.20 | no binding |
| ACI-8016-31F10C5-AB1 | 0.24 | 0.30 | 0.18 | no binding |
| ACI-8016-19E6D4-AB1* | 0.10 | no binding | no binding | 0.09 |
| ACI-8016-3E6B11-AB1* | 0.15 | 0.15 | no binding | 0.13 |
| ACI-8016-11A3F3-AB1* | 0.11 | 0.63 | no binding | 0.12 |
| ACI-8016-14G5B8-AB1 * | 0.09 | 0.16 | no binding | 0.17 |
| ACI-8016-22A10F8-AB1* | 0.11 | 0.09 | 0.15 | no binding |
| ACI-8016-27A1G4-AB1* | 0.15 | 0.11 | no binding | 0.13 |
| ACI-8016-29C5E11-AB1* | 0.16 | 0.14 | no binding | 0.14 |
| ACI-8016-7G3B5-AB1* | 0.19 | 0.17 | 0.16 | no binding |
| ACI-8016-2504F3D9-AB1 | 0.19 | 1.09 | no binding | 0.13 |
| ACI-8016-2516A8C6-AB1* | 0.26 | 0.26 | 0.16 | no binding |
| ACI-8016-2602H6F10-AB1* | 11.1 | 4.52 | 2.52 | no binding |
| ACI-8016-2609F4A9-AB1 | 0.12 | 0.30 | no binding | 0.11 |
| ACI-8016-2610H7D3-AB1 | 0.19 | 0.27 | 0.16 | no binding |
| ACI-8016-2614C3B2-AB1* | 1.98 | 2.67 | low binding | 2.21 |
| ACI-8016-2617C3A8-AB1 | 0.16 | 0.23 | 0.16 | no binding |
| ACI-8016-2622E12F11-AB1 | 0.11 | 0.16 | no binding | 0.12 |
| ACI-8016-2626B9D3-AB1 | 0.14 | 0.38 | 0.14 | no binding |
| ACI-8016-2629E8D1-AB1 | 0.11 | 0.22 | no binding | 0.12 |

N.D.: Not determined due to dilution curves not being in range for a non-constrain variable slope 4 parameter fit;
NA: not tested;
*data generated using hybridoma purified mAb

### Example 3. Characterization of ASC Antibodies Binding Specificity by Immunoblotting

### Methods

### Cell Lysate Sample Preparation

The differentiation of human monocytes and human ASC-KO monocytes into macrophages was conducted in presence of 10 ng/mL phorbol 12-myristate 13-acetate (PMA) overnight. The differentiated cells were then exposed to lipopolysaccharide (LPS) 10 ng/mL for 3 hours. J774.A1 macrophages were stimulated with LPS overnight. Following LPS stimulation, the cells were homogenized in lysis buffer supplemented with 1 mM of PMSF. Cell lysates were vortexed and kept on ice for 15 min before being centrifuged at 20,000 g for 10 min. The total protein concentration of the soluble extract of the cell lysate samples was assessed using the Pierce™ BCA assay kit. Concentrations were adjusted to 1 μg/μL.

### Western Blot

g of cell lysates and 30 ng of recombinant ASC proteins were mixed with 4× Sample Loading buffer and 0.1 mM DTT final concentration and boiled for 5 min at 95° C. Samples were loaded on a 4-12% Bis-Tris gel and migrated at 150 Volt (V) for 45 min. Proteins were transferred on a nitrocellulose membrane using the iBlot 2 system (20 mA, 7 min). Membranes were blocked for 1 hour at room temperature (RT) under agitation in LI-COR blocking buffer. Primary antibodies were diluted in LI-COR blocking buffer diluted one to two in phosphate-buffered saline (PBS). Membranes were incubated overnight at 4° C. in the antibody mix. Primary antibodies used were rabbit anti-ASC AL177 (Adipogen, cat. n° AG-25B-0006) and the ASC mAbs at 1 μg/mL. Membranes were washed three times 5 min in PBS 0.1% Tween-20 under agitation. Secondary antibodies (Donkey anti-mouse IRDye800CW and Goat anti-rabbit IRDye®680CW) were diluted 1:10′000 in the same buffer as primary antibodies and membranes were incubated for 1 hour at room temperature (RT) under constant agitation. Membranes were scanned on the LI-COR Odyssey scanner.

### Results

mAbs were analyzed by WB on cellular soluble extracts of LPS-primed human and mouse macrophages (FIG. 1). The commercially available ASC antibody AL177 was used as a reference antibody due to its human and mouse cross-reactivity. Human and mouse recombinant ASC proteins were used as positive controls for ASC detection. The difference in molecular weight is due to the presence of tags in recombinant proteins. Human ASC KO monocytes lysates were used as negative control for ASC detection. Out of the 23 mAbs tested, 9 cross-reacted with both human and mouse cell lysate samples (ACI-8016-18F4C12-AB1, ACI-8016-23E5F7-AB1, ACI-8016-23E5F7-AB2, ACI-8016-26A1G2-AB1, ACI-8016-32B6C7-AB1, ACI-8016-22D3A6-AB1, ACI-8016-31F10C5-AB1, ACI-8016-2610H7D3-AB1, ACI-8016-2617C3A8-AB1). Seven displayed only human binding specificity (ACI-8016-402H11C9-Ab1, ACI-8016-417E12A8-AB1, ACI-8016-413G10A5-AB1, ACI-8016-407E10A9-AB1, ACI-8016-203G8B10-AB1, ACI-8016-401H9B7-AB1, ACI-8016-2504F3D9-AB1) while the remaining mAbs presented peculiar binding profile by cross-reacting with all samples except J774.A1 cell lysates (ACI-8016-416E6G4-AB1, ACI-8016-421B10C12D2-AB1, ACI-8016-417E12A8-AB1, ACI-8016-401H9B7-AB1, ACI-8016-2609F4A9-AB1, ACI-8016-2626B9D3-AB1, ACI-8016-2629E8D1-AB1). Lower molecular weight (MW) band (˜15 kDa) observed in a human monocytic cell lysates probably represents a splice isoform or a degradation product of ASC and appears specific as it is absent in lysates from human-ASC-KO monocytes. No pronounced signal was detected for products with unexpected MW indicating the specificity of tested mAbs. The results for ASC detection in cell lysates are summarized in Table 2. Any differences between binding profiles by WB and ELISA might be due to the non-linear epitope of some mAbs or to differences between recombinant and native endogenous proteins.

TABLE 2
ASC detection in cell lysates

| | Mouse ASC | Human ASC |
|---|---|---|
| | target | target |
| Antibody name | engagement | engagement |
| ACI-8016-416E6G4-AB1 | − | + |
| ACI-8016-402H11C9-Ab1 | − | + |
| ACI-8016-421B10C12D2-AB1 | − | + |
| ACI-8016-417E12A8-AB1 | − | + |
| ACI-8016-413G10A5-AB1 | − | + |
| ACI-8016-407E10A9-AB1 | − | + |
| ACI-8016-203G8B10-AB1 | − | + |
| ACI-8016-401H9B7-AB1 | − | + |
| ACI-8016-18F4C12-AB1 | + | + |
| ACI-8016-23E5F7-AB1 | + | + |
| ACI-8016-23E5F7-AB2 | + | + |
| ACI-8016-26A1G2-AB1 | + | + |
| ACI-8016-32B6C7-AB1 | + | + |
| ACI-8016-22D3A6-AB1 | + | + |
| ACI-8016-31F10C5-AB1 | + | + |
| ACI-8016-2504F3D9-AB1 | − | + |
| ACI-8016-2609F4A9-AB1 | − | + |
| ACI-8016-2610H7D3-AB1 | + | + |
| ACI-8016-2617C3A8-AB1 | + | + |
| ACI-8016-2626B9D3-AB1 | − | + |
| ACI-8016-2629E8D1-AB1 | − | + |

+ indicates positive signal;
− indicates no signal

### Example 4. SPR Based Affinity Determination for ASC mAbs

### Methods

Affinity and avidity mode measurements were performed on a Biacore T200 instrument (Cytiva, formerly GE Healthcare). hASC, mASC and MBP-hASC Immobilization: The instrument was primed with running buffer (10×PBS-P+ diluted to 1× in Milli-Q water) and flow channels (Fe) 1-4 of a CM5 Series S sensor chip were activated with a fresh solution of EDC/NHS at 5 μL/min for 420 sec (Amine Coupling Kit, 1:1 ratio of both reagents). hASC was diluted in 10 mM sodium acetate pH 4.0 to a final concentration of 50 μg/mL and injected for 600 sec on Fc2 to a final level of 500 RU. mASC was diluted in 10 mM sodium acetate pH 4.0 to a final concentration of 2 μg/mL and injected for 300 sec on Fc3 to a final level of 570 RU. MBP-mASC was diluted in 10 mM sodium acetate pH 4.0 to a final concentration of 20 μg/mL and injected for 180 sec on Fc4 to a final level of 660 RU. Next, all unreacted activated ester groups were quenched with 1 M ethanolamine for 420 sec. Two successive regenerations of 10 mM glycine-HCl pH 1.7 for 30 sec were conducted to remove any non-covalently bound ASC.

Binding Kinetics: Single-cycle kinetics were performed with a surface regeneration between each cycle. Prior to analysis, two Startup Cycles were run. ASC mAbs were injected in increasing concentration from 1.2-100 nM, prepared from a 3-fold serial dilution in running buffer, with a contact time of 300 sec and a dissociation time of 900 sec at a flow rate of 30 μL/min. Each successive mAb was preceded by a regeneration step using 10 mM glycine-HCl pH 1.7 with a contact time of 30 sec at 10 μL/min, followed by a stabilization period of 300 sec. Results obtained from single-cycle kinetics were double-referenced using the blank Fc 1 and a buffer cycle and evaluated by Biacore T200 evaluation software with a 1:1 kinetic fit model (with RI and global Rmax). The following kinetic parameters were obtained: Association rate constant (ka), dissociation rate constant (kd), affinity constant (KD) and saturation response (Rmax). Fitting was rejected if less than three curves could be fit. The commercial antibodies F-9 (anti-hASC) and 2EI-7 (anti-mASC) were used as controls at the start and end of the experiment.

Capture mAb Immobilization: The instrument was primed with running buffer (10×PBS-P+ diluted to 1× in Milli-Q water). Flow-cells 1-2 of all 8 Fc,s on a CM5 Series S sensor chip were activated with a fresh solution of EDC/NHS at 10 μL/min for 420 sec (Amine Coupling Kit, 1:1 ratio both reagents) and 30 μg/mL goat anti-mouse antibody diluted in 10 mM sodium acetate pH 5.0 was captured at a flow rate of 10 μL/min for 420 sec. Next, all unreacted activated ester groups were quenched with 1 M ethanolamine for 420 sec. Any non-covalently bound antibodies were removed by two successive regenerations of 10 mM glycine-HCl pH 1.7 for 30 sec. Immobilization levels were evaluated after immobilization (13000 RU for all 16 flow-cells).

ASC mAb Capture and Binding Kinetics: Each cycle started with non-covalent capture of ASC mAbs (listed in Table 1) which were diluted in running buffer to a final concentration of 2 μg/mL and injected for 90 sec with a flow rate of 10 μL/min. Eight mAbs were captured on flow-cell 2 at the same time, leaving flow-cell 1 as a blank control. Capture levels were evaluated following a 120 sec stabilization period after each mAb injection and ranged from 300 to 460 RU (see Table 3). The commercial antibodies F-9 (anti hASC, SantaCruz BioTechnologies, cat. n° sc-271054) and 2EI-7 (anti mASC) were used as controls at the start and end of the experiment.

Binding affinity of mAbs for hASC and mASC was assessed using a single-cycle kinetics method. Prior to analysis, two startup cycles were run. Injections of hASC and mASC, increasing in concentration from 2.2-180 nM (hASC) or from 1.5-120 nM (mASC) prepared from serial 3-fold dilutions, were performed with a contact time of 300 sec at a flow rate of 30 μL/min, followed by a dissociation phase of 600 sec. Regeneration of the goat anti-mouse antibody was achieved by injection of 10 mM glycine-HCl pH 1.7 at a flow rate of 10 μL/min for 120 sec, followed by a stabilization period of 300 sec. For blank injections running buffer was injected under the same conditions. Results obtained from single-cycle kinetics were double-referenced using the blank flow-cell 1 and a buffer cycle and evaluated using the Biacore 8K evaluation software with 1:1 kinetic fit model (global Rmax). The following kinetic parameters were obtained: Association rate constant (ka), dissociation rate constant (kd), affinity constant (KD) and saturation response (Rmax). Fitting was rejected if less than three curves could be fit.

### Results

The affinities, from avidity mode measurements, of mAbs for hASC and mASC were analyzed in this study. hASC and mASC were covalently immobilized and single-cycle kinetics were performed with increasing mAb concentrations ranging from 1.2 nM to 100 nM. Obtained sensorgrams were fit with a 1:1 kinetic fit model. mAbs with a signal below 20 RU at the highest concentration were considered as “low binders”. Responses with no concentration dependence or with signal below 5 RU at the highest concentration were considered as “non-binders”. Table 3 reports binding constants evaluated in avidity mode using a 1:1 kinetic fit model including commercial mAbs as quality controls of the biosensor at the start and end of the run. All mAbs tested showed binding to hASC in avidity mode with KD values ranging from below 0.01 nM to 10.52 nM. Three out of 10 tested mAbs showed binding to mASC, under the conditions tested, with KD values ranging from 4.88 nM to 16.1 nM. All mAbs showed preferential binding to hASC with 5-10-fold-higher KD values than for mASC. No mouse-specific mAbs were detected.

TABLE 3
Binding parameters from SPR single-cycle kinetics avidity mode measurements.

| | | hASC | | | mASC | | |
|---|---|---|---|---|---|---|---|
| | | ka | kd | KD | ka | kd | KD |
| Antibody | | (1/Ms) | (1/s) | (nM) | (1/Ms) | (1/s) | (nM) |
| ACI-8016-416E6G4-AB1 | Mean | 3.9E+04 | 2.7E−05 | 0.85 | 3.5E+04 | 1.3E−04 | 4.88 |
| | SD | 1.8E+04 | 9.9E−06 | 0.66 | 2.2E+04 | 2.1E−05 | 3.42 |
| ACI-8016-402H11C9-Ab1 | Mean | NA | NA | 10.52 | no binding | | |
| | SD | NA | NA | 0.71 | NA | NA | NA |
| ACI-8016-203B12C3-AB1 | Mean | NA | NA | 10.21 | NA | NA | Low |
| | | | | | | | binding |
| | SD | NA | NA | 1.58 | NA | NA | 2.22 |
| ACI-8016-421B10C12D2-AB1 | Mean | 1.9E+05 | 2.2E−07 | <0.01 | no binding | | |
| | SD | 4.9E+03 | 4.7E−08 | NA | NA | NA | NA |
| ACI-8016-417E12A8-AB1 | Mean | 1.1E+05 | 4.1E−07 | <0.01 | no binding | | |
| | SD | 1.4E+03 | 4.0E−07 | NA | NA | NA | NA |
| ACI-8016-413G10A5-AB1 | Mean | 9.2E+04 | 1.2E−06 | 0.01 | no binding | | |
| | SD | 7.8E+02 | 1.3E−06 | 0.01 | NA | NA | NA |
| ACI-8016-407E10A9-AB1 | Mean | 1.1E+05 | 5.6E−07 | <0.01 | no binding | | |
| | SD | 1.4E+03 | 2.6E−08 | NA | NA | NA | NA |
| ACI-8016-203G8B10-AB1 | Mean | 8.3E+04 | 2.9E−05 | 0.35 | no binding | | |
| | SD | 9.9E+02 | 1.8E−06 | 0.02 | NA | NA | NA |
| ACI-8016-401H9B7-AB1 | Mean | 7.4E+04 | 2.3E−07 | <0.01 | no binding | | |
| | SD | 1.6E+03 | 2.4E−07 | NA | NA | NA | NA |
| ACI-8019-2314F6H11-AB1 | Mean | 8.8E+04 | 8.0E−05 | 0.91 | NA | NA | 16.10 |
| | SD | 2.1E+02 | 1.2E−05 | 0.14 | NA | NA | 3.54 |

NA, not applicable.

In addition to avidity mode measurements, affinities of mAbs for hASC and mASC were also analyzed in affinity mode. mAbs were non-covalently captured by Fe-region on the sensor surface and single-cycle kinetics were performed with increasing hASC concentrations ranging from 2.2 nM to 180 nM and mASC concentrations ranging from 1.5 nM to 120 nM. Obtained sensorgrams were fit with a 1:1 kinetic fit model. mAbs with a signal below 20 RU at the highest concentration were considered as “low binders”. Responses with no concentration dependence or with signal below 5 RU at the highest concentration were considered as “non-binders”. Table 4 reports binding constants evaluated in affinity mode using a 1:1 kinetic fit model.

TABLE 4
Fitting parameters from SPR single-cycle kinetics affinity mode measurements.

| | | hASC | | | mASC | | |
|---|---|---|---|---|---|---|---|
| Antibody | | ka (1/Ms) | kd (1/s) | KD (nM) | ka (1/Ms) | kd (1/s) | KD (nM) |
| ACI-8016-416E6G4- | Mean | 2.8E+05 | 2.5E−03 | 14.86 | 3.4E+04 | 5.3E−04 | 14.52 |
| AB1 | SD | 3.8E+05 | 3.0E−03 | 9.08 | 1.4E+04 | 4.0E−04 | 5.93 |
| ACI-8016- | Mean | 3.6E+05 | 1.9E−04 | 0.55 | no binding | | |
| 402H11C9-Ab1 | SD | 6.3E+04 | 4.7E−05 | 0.19 | NA | NA | NA |
| ACI-8016- | Mean | NA | NA | 41.99 | no binding | | |
| 203B12C3-AB1 | SD | NA | NA | 8.81 | NA | NA | NA |
| ACI-8016- | Mean | 4.7E+05 | 1.7E−04 | 0.36 | no binding | | |
| 421B10C12D2-AB1 | SD | NA | NA | NA | NA | NA | NA |
| ACI-8016- | Mean | 2.5E+05 | 2.9E−05 | 0.12 | no binding | | |
| 417E12A8-AB1 | SD | NA | NA | NA | NA | NA | NA |
| ACI-8016- | Mean | 1.6E+05 | 2.2E−05 | 0.13 | no binding | | |
| 413G10A5-AB1 | SD | NA | NA | NA | NA | NA | NA |
| ACI-8016- | Mean | 1.3E+05 | 1.5E−05 | 0.11 | no binding | | |
| 407E10A9-AB1 | SD | NA | NA | NA | NA | NA | NA |
| ACI-8016- | Mean | 1.3E+05 | 1.7E−04 | 1.34 | no binding | | |
| 203G8B10-AB1 | SD | NA | NA | NA | NA | NA | NA |
| ACI-8016-401H9B7- | Mean | 9.6E+04 | 3.2E−08 | <0.01 | no binding | | |
| AB1 | SD | NA | NA | NA | NA | NA | NA |
| ACI-8016-18F4C12- | Mean | 6.21E+05 | 4.42E−04 | 0.70 | NA | NA | 54.54 |
| AB1 | SD | 3.33E+04 | 1.29E−04 | 0.17 | NA | NA | 8.24 |
| ACI-8016-23E5F7- | Mean | NA | NA | *27.06 | 2.61E+05 | 2.64E−03 | 10.35 |
| AB1_{—} | SD | NA | NA | *1.05 | 3.59E+04 | 6.72E−05 | 1.68 |
| ACI-8016-23E5F7- | Mean | 4.87E+05 | 1.50E−03 | 3.11 | 3.84E+05 | 6.15E−04 | 1.62 |
| AB2 | SD | 4.11E+04 | 1.35E−06 | 0.27 | 3.46E+04 | 3.95E−05 | 0.25 |
| ACI-8016-26A1G2- | Mean | 4.20E+04 | 8.05E−05 | 1.87 | 2.76E+04 | 1.01E−04 | 3.81 |
| AB1 | SD | 2.18E+03 | 4.43E−05 | 0.00 | 1.99E+03 | 5.16E−05 | 2.15 |
| ACI-8016-32B6C7- | Mean | 1.92E+05 | 2.01E−04 | 1.06 | NA | NA | 36.87 |
| AB1 | SD | 2.32E+04 | 1.31E−05 | 0.06 | NA | NA | 3.71 |
| ACI-8016-22D3A6- | Mean | 6.90E+05 | 2.64E−04 | 0.38 | 5.98E+05 | 6.90E−04 | 1.16 |
| AB1 | SD | 1.00E+04 | 3.70E−06 | 0.01 | 4.06E+04 | 3.33E−05 | 0.13 |
| ACI-8016-31F10C5- | Mean | 1.23E+06 | 1.99E−04 | 0.16 | 8.86E+05 | 3.14E−04 | 0.36 |
| AB1 | SD | 1.15E+05 | 4.91E−05 | 0.02 | 5.60E+04 | 1.60E−06 | 0.02 |
| ACI-8016- | Mean | 8.93E+04 | 7.23E−04 | 8.30 | 2.37E+04 | 1.62E−03 | No binding |
| 2504F3D9-AB1 | SD | 4.92E+03 | 2.88E−04 | 3.69 | 2.26E+04 | 8.54E−04 | NA |
| ACI-8016- | Mean | 2.97E+05 | 7.83E−03 | 24.92 | NA | NA | *167.96 |
| 2609F4A9-AB1 | SD | 6.83E+04 | 3.57E−03 | 6.30 | NA | NA | *25.26 |
| ACI-8016- | Mean | 7.47E+05 | 2.01E−04 | 0.27 | 8.31E+05 | 3.39E−04 | 0.41 |
| 2610H7D3-AB1 | SD | 1.07E+05 | 3.00E−05 | 0.00 | 9.87E+03 | 5.97E−05 | 0.07 |
| ACI-8016- | Mean | 8.10E+05 | 1.18E−04 | 0.15 | 1.09E+06 | 1.62E−04 | 0.15 |
| 2617C3A8-AB1 | SD | 6.11E+04 | 4.68E−06 | 0.02 | 5.37E+03 | 2.21E−05 | 0.02 |
| ACI-8016- | Mean | 2.35E+05 | 7.86E−04 | 3.66 | NA | NA | *176.23 |
| 2622E12F11-AB1 | SD | 2.96E+04 | 4.68E−04 | 2.45 | NA | NA | *8.17 |
| ACI-8016- | Mean | 9.34E+05 | 3.61E−03 | 7.55 | NA | NA | *34.40 |
| 2626B9D3-AB1 | SD | 6.71E+05 | 2.73E−04 | 5.13 | NA | NA | *1.69 |
| ACI-8016- | Mean | 1.40E+05 | 1.82E−04 | 1.38 | NA | NA | *133.57 |
| 2629E8D1-AB1 | SD | 1.33E+04 | 9.66E−05 | 0.82 | NA | NA | *9.29 |

*For hASC, ACI-8016-23E5F7-AB1 and for mASC, ACI-8016-18F4C12-AB1, ACI-8016-32B6C7-AB1, ACI-8016-2609F4A9-AB1, ACI-8016-2622E12F11-AB1, ACI-8016-2626B9D3-AB1and ACI-8016-2629E8D1-AB1 data were obtained from steady-state fit model and reflect apparent KD values due to heterogenous binding.

All mAbs showed binding to hASC in affinity mode under the conditions tested with KD values ranging from below 0.01 nM to 42 nM. 14 mAbs showed binding to mASC with KD values ranging from 0.15 nM to 176.2 nM. Of these 9 mAbs had KD values comparable to KD values for hASC (<5-fold difference), and 5 mAbs (ACI-8016-18F4C12-AB1, ACI-8016-32B6C7-AB1, ACI-8016-2609F4A9-AB1, ACI-8016-2622E12F11-AB1 and ACI-8016-2629E8D1-AB1_rmG2a_01) showed preferential binding to hASC. No mouse-specific mAbs were detected.

### Example 5. Epitope Binning by Bio-Layer Interferometry (BLI)

### Method

To determine the epitope coverage and diversity of the antibody panel, an epitope binning experiment was performed. Competitive epitope binning of ASC mAbs was performed on OctetQKe (ForteBio) in “in-Tandem” mode. Biotinylated PYD or CARD domain were captured on streptavidin biosensor (forteBio, cat #18-5020) for 180 s at 12.5 nM. After immobilization, binning experiment was performed by sequential incubation of a first antibody at saturating concentration, typically 300 nM for 600 s, followed by a second antibody, so called competing antibody, at 150 nM for 300 s. After each competition cycle, biosensors were regenerated by three consecutive incubations of 5 s in 0.1 M Glycine pH 1.5 followed by a neutralization step of 30 s in PBS, 0.1% BSA, 0.02% Tween.

All antibodies were tested in a matrix fashion and in both assay orientations. For each competition cycle, a blank run was performed using 1×PBS, 0.1% BSA, 0.02% Tween to determine baseline and maximum binding level. Data were analyzed using the Data Analysis HT 11.1 software (Bioforte). Antibodies with antigen responses below 0.1 nm, and antibodies that did not self-compete were excluded from the analysis. Competing mAb responses were normalized relative to the competing antibody alone binding response. Antibodies with normalized responses <15% were classified as blocker, those with normalized responses >15% were classified as non-blockers. A bin was defined as a group of antibodies competing in both orientations.

### Results

Tables 5 and 6 show normalized values for the binding of the indicated antibodies to the PYD and CARD respectively. Antibodies at saturating concentration are represented as rows and competing antibodies as columns. ACI-8016-416E6G4-AB1 binding signal was too low to be tested as saturating antibody however it could be used as competing antibody.

TABLE 5
Normalized binding response of epitope binning experiment against PYD

| | ACI- | ACI- | ACI- | ACI- | ACI- | | | | ACI- |
|---|---|---|---|---|---|---|---|---|---|
| | 8016- | 8016- | 8016- | 8016- | 8016- | ACI-8016- | ACI-8016- | ACI-8016- | 8016- |
| | 23E5F7- | 26A1G2- | 22D3A6- | 31F10C5- | 401H9B7- | 2610H7D3- | 2617C3A8- | 2626B9D3- | 416E6G4- |
| Ab# | AB2 | AB1 | AB1 | AB1 | AB1 | AB1 | AB1 | AB1 | AB1 |
| ACI-8016- | 2 | −3 | 3 | 2 | 59 | 2 | 3 | 62 | −12 |
| 23E5F7- | | | | | | | | | |
| AB2 | | | | | | | | | |
| ACI-8016- | 18 | 3 | 23 | 19 | 63 | 22 | 23 | 33 | 14 |
| 26A1G2- | | | | | | | | | |
| AB1 | | | | | | | | | |
| ACI-8016- | 1 | 0 | 2 | 1 | 73 | 3 | 2 | 74 | 2 |
| 22D3A6- | | | | | | | | | |
| AB1 | | | | | | | | | |
| ACI-8016- | 2 | 0 | 3 | 1 | 79 | 2 | 3 | 49 | 4 |
| 31F10C5- | | | | | | | | | |
| AB1 | | | | | | | | | |
| ACI-8016- | 83 | 24 | 90 | 95 | 3 | 108 | 112 | 3 | 47 |
| 401H9B7- | | | | | | | | | |
| AB1 | | | | | | | | | |
| ACI-8016- | 0 | 1 | 2 | 2 | 73 | 1 | 0 | 80 | 6 |
| 2610H7D3- | | | | | | | | | |
| AB1 | | | | | | | | | |
| ACI-8016- | 1 | 2 | 1 | 2 | 75 | 0 | 2 | 88 | 4 |
| 2617C3A8- | | | | | | | | | |
| AB1 | | | | | | | | | |
| ACI-8016- | 58 | 6 | 66 | 29 | 4 | 73 | 98 | 2 | −13 |
| 2626B9D3- | | | | | | | | | |
| AB1 | | | | | | | | | |

TABLE 6
Normalized binding response of epitope binning experiment against CARD

| | ACI-8016- | ACI-8016- | ACI-8016- | ACI-8016- | ACI-8016- | ACI-8016- |
|---|---|---|---|---|---|---|
| | 2504F3D9- | 2609F4A9- | 2622E12F11- | 2629E8D1- | 32B6C7- | 18F4C12- |
| Ab# | Ab1 | Ab1 | Ab1 | Ab1 | Ab1 | Ab1 |
| ACI-8016-2504F3D9-Ab1 | 0 | 17 | 19 | 20 | 21 | 76 |
| ACI-8016-2609F4A9-Ab1 | 0 | 0 | 103 | 0 | 7 | 96 |
| ACI-8016-2622E12F11-Ab1 | −2 | 74 | 0 | 87 | 91 | 18 |
| ACI-8016-2629E8D1-Ab1 | −2 | −2 | 102 | 0 | 3 | 95 |
| ACI-8016-32B6C7-Ab1 | −3 | −3 | 106 | −2 | 0 | 97 |
| ACI-8016-18F4C12-Ab1 | 33 | 68 | −3 | 69 | 78 | 0 |

TABLE 7
Summary table for the repartition of antibodies in different bins.

| | PYD | CARD |
|---|---|---|
| Bin1 | ACI-8016-23E5F7-AB2 | ACI-8016-32B6C7-AB1 |
| | ACI-8016-22D3A6-AB1 | ACI-8016-2609F4A9-AB1 |
| | ACI-8016-31F10C5-AB1 | ACI-8016-2629E8D1-AB1 |
| | ACI-8016-2610H7D3-AB1 | |
| | ACI-8016-2617C3A8-AB1 | |
| Bin 2 | ACI-8016-401H9B7-AB1 | ACI-8016-18F4C12-AB1 |
| | ACI-8016-2626B9D3-AB1 | ACI-8016-2622E12F11-AB1 |
| Bin3 | ACI-8016-416E6G4-AB1 | ACI-8016-2504F3D9-AB1 |
| | ACI-8016-26A1G2-AB1 | |

### Example 6. Analysis of Target Engagement of ASC Antibodies in Human Monocytic Cell Line and Mouse J774 Macrophages by Immunofluorescent Labeling

### Methods

### Cell Culture and Treatments to Induce ASC Speck Assembly

### Human Monocytic Cell Line

Human monocytic cell line (ACC16, DMSZ) was cultured in RPMI 1640 supplemented with 10% heat inactivated fetal bovine serum (FBS; Gibco, Qualified, HI, 10500-064) and 1% penicillin/streptomycin (P/S, 100 μg/mL each (Gibco, 10378-016)). Human monocytes were seeded at 75×10⁴ cells/well in the 60-inner wells of 96-well cellcarrier ultraplates (Perkin Elmer, 6055302) and then differentiated with 10 ng/mL phorbol 12-myristate 13-acetate (PMA; Sigma, P1585) overnight at 37° C., 5% C02. The following day, the medium was replaced, and PMA differentiated human monocytes were primed with 10 ng/mL LPS from Escherichia coli (Enzo LifeScience, #ALX-581-013-L002) for 3h to induce the synthesis of pro-Interleukin-10 (pro-IL-1β). Medium was removed and cells were treated for 30 min with 50 μM of caspase-1 inhibitor Z-Val-Ala-DL-Asp-fluoromethylketone ((z-VAD), company, ref?) to completely inhibit NLRP3 inflammasome and prevent cell death. Cells were then stimulated with Nigericin (Invivogen, #tlrl-nig-5) at 10 μM for 1 h to induce NLRP3 inflammasome activation and ASC speck assembly. After Inflammasome activation, the cells were washed with DPBS and fixed with PFA 4% diluted in DPBS during 20 min at RT and washed 3 time with DPBS. For control conditions cells were differentiated with 10 ng/mL phorbol 12-myristate 13-acetate (PMA) overnight at 37° C., 5% C02. The following day, the medium was replaced, and PMA differentiated human monocytes were primed with 10 ng/mL LPS for 3h. In parallel of the inflammasome activated cells, the cells were washed with DPBS and fixed with PFA 4% diluted in DPBS during 20 min at RT and washed 3 time with DPBS.

### J774.A1 Cell Lines

J774.A1 cells were cultured in DMEM supplemented with 10% heat inactivated fetal bovine serum (FBS) and 1% P/S. J774.A1 cells were seeded at 50×10⁴ cells/well in the 60-inner wells of 96-well 96-well Black/Clear Flat Bottom TC-treated (Falcon®, 353219), were primed with 100 ng/mL LPS from Escherichia coli to induce the synthesis of pro-Interleukin-1p (pro-IL-1β) and incubated overnight at 37° C., 5% CO₂. The following day, the medium was replaced, and cells were treated 30 min with 50 μM of caspase-1 inhibitor (z-VAD) to completely inhibit NLRP3 inflammasome and prevent cell death. Cells were then stimulated with ATP (Invivogen, #tlrl-atp) at 4 mM for 1 h to induce NLRP3 inflammasome activation and ASC speck assembly. After Inflammasome activation, the cells were washed with DPBS and fixed with PFA 4% diluted in DPBS during 20 min at RT and wash 3 time with DPBS. For control conditions cells were differentiated with 10 ng/mL PMA overnight at 37° C., 5% C02. The following day, the medium was replaced, and PMA differentiated human monocytes were primed with 10 ng/mL LPS for 3h. In parallel of the inflammasome activated cells, the cells were washed with DPBS and fixed with PFA 4% diluted in DPBS during 20 min at RT and washed 3 time with DPBS.

### Immunofluorescence Labeling

Fixed cells were permeabilized with DPBS-0.25% Triton® X-100 (Sigma, 23,472-9) during 10 min and blocked with DPBS-0.25% Triton X-100-5% BSA (MACS @BSA stock solution, Miltenyi, 130-091-376) during 1 h at RT. ASC mAbs were diluted 1/10 in DPBS and further diluted 1/100 in DPBS-0.25% Triton X-100-5% BSA to a final concentration of 1 μg/mL. After removing the blocking solution, 50 μL of mAbs, human-specific rabbit polyclonal AL-177 antibody for human monocytes diluted at 1 μg/mL, mouse-specific rabbit monoclonal D2W8U antibody for J774 cells diluted at 0.2 μg/mL, or IgG2a isotype control antibody diluted at 1 μg/mL, were added, and incubated overnight at 4° C. with a gentle agitation. The following day, the cells were washed 3 times 10 min each with DPBS-0.25% Triton X-100. Then the secondary antibodies goat anti-mouse DyLight 488 nm (ThermoFisher Scientific, 35502) for all ASC mAbs or goat anti-rabbit DyLight 488 nm (ThermoFisher Scientific, 35552) for commercial AL-177 and D2W8U antibodies were added and incubated 1 h at RT in the dark. Cells were washed 3 times with DPBS-0.25% Triton X-100, 10 min each. Nuclei were stained during 10 min at RT with NucBlue™ fixed cell stain Ready Probes reagent (Invitrogen, R37606), 2 drops per 1 mL of DPBS-0.25% Triton X-100. Cells were washed 3 times with DPBS-0.25% Triton X-100, 10 min each. After a last wash with DPBS, 200 μL of DPBS was added and plates were stored at 4° C. until the day of analysis. Image acquisition was performed at the Biomolecular Screening Facility of EPFL with the GE Healthcare IN Cell Analyzer 2200 automated microscope. Image analysis was performed using IN Cell Analyzer 2200 and ImageJ softwares.

### Results

### Target Engagement on Human ASC Specks in a Human Monocytic Cell Line

No ASC specks were detected in control conditions. The isotype control IgG2a showed no specific staining. Positive reference antibody AL177 showed diffuse staining in the cytoplasm along with bright and intense dots corresponding to ASC specks. Table 8 summarizes the staining results and representative images are shown on FIG. 2. All the tested ASC mAbs showed labeling of hASC. ASC specks and a light cytoplasm staining was observed for ACI-8016-402H11C9-Ab1, ACI-8016-23E5F7-AB1, ACI-8016-2504F3D9-AB1, ACI-8016-2609F4A9-AB1, ACI-8016-2622E12F11-AB1 and ACI-8016-2626B9D3-AB1. ASC specks and an intermediate cytoplasm staining was observed with ACI-8016-421B10C12D2-AB1, A-8016-203G8B10-AB1, ACI-8016-18F4C12-AB1, ACI-8016-23E5F7-AB2, ACI-8016-22D3A6-AB1 and ACI-8016-2629E8D1-AB1. ASC specks and a strong cytoplasm staining was observed for ACI-8016-413G10A5-AB1, ACI-8016-407E10A9-AB1, ACI-8016-26A1G2-AB1, ACI-8016-31F10C5-AB1, A-8016-2610H7D3-AB1 and ACI-8016-2617C3A8-AB1. Preferential staining of aggregated form of ASC was observed for ACI-8016-203B12C3-AB1. Preferential staining for cytoplasmic ASC was observed for ACI-8016-401H9417-AB1.

TABLE 8
Results of immunofluorescence staining observed
with the ASC mAbs in a human monocytic cell line.

| | Localization and relative intensity of staining | | |
|---|---|---|---|
| | | Detection of | Detection of ASC |
| Antibody name | Description | ASC specks | in cytoplasm |
| ACI-8016-402H11C9-Ab1 | ASC speck & cytoplasm | ++ | + |
| ACI-8016-203B12C3-AB1 | ASC speck | +++ | − |
| ACI-8016-421B10C12D2-AB1 | ASC speck & cytoplasm | +++ | ++ |
| ACI-8016-417E12A8-AB1 | ASC speck & cytoplasm | + | +++ |
| ACI-8016-413G10A5-AB1 | ASC speck & cytoplasm | ++ | +++ |
| ACI-8016-407E10A9-AB1 | ASC speck & cytoplasm | +++ | +++ |
| ACI-8016-203G8B10-AB1 | ASC speck & cytoplasm | +++ | ++ |
| ACI-8016-401H9B7-AB1 | ASC speck & cytoplasm | + | ++++ |
| ACI-8016-18F4C12-AB1 | ASC speck & cytoplasm | +++ | ++ |
| ACI-8016-23E5F7-AB1 | ASC speck & cytoplasm | +++ | + |
| ACI-8016-23E5F7-AB2 | ASC speck & cytoplasm | +++ | ++ |
| ACI-8016-26A1G2-AB1 | ASC speck & cytoplasm | ++ | +++ |
| ACI-8016-22D3A6-AB1 | ASC speck & cytoplasm | +++ | ++ |
| ACI-8016-31F10C5-AB1 | ASC speck & cytoplasm | +++ | +++ |
| ACI-8016-2504F3D9-AB1 | ASC speck & cytoplasm | ++ | + |
| ACI-8016-2609F4A9-AB1 | ASC speck & cytoplasm | +++ | + |
| ACI-8016-2610H7D3-AB1 | ASC speck & cytoplasm | +++ | +++ |
| ACI-8016-2617C3A8-AB1 | ASC speck & cytoplasm | +++ | +++ |
| ACI-8016-2622E12F11-AB1 | ASC speck & cytoplasm | +++ | + |
| ACI-8016-2626B9D3-AB1 | ASC speck & cytoplasm | +++ | + |
| ACI-8016-2629E8D1-AB1 | ASC speck & cytoplasm | +++ | ++ |
| ACI-8016-2610H7D3-AB1 | ASC speck & cytoplasm | +++ | +++ |
| ACI-8016-2617C3A8-AB1 | ASC speck & cytoplasm | +++ | +++ |

−, absent;
+, weak;
++, medium;
+++, abundant;
++++, very abundant

Target Engagement Analysis of ASC mAhs on Mouse Macrophage J774.A1 Cells

No staining was observed when the isotype control IgG2a was used. Positive reference antibodies 2EI-7 and D2W8U, both displayed intense dots corresponding to ASC specks. The antibody D2W8U showed a bright and diffuse cytoplasmic staining. Table 9 summarizes the staining results. ASC specks and a light cytoplasm staining was observed with ACI-8016-18F4C12-AB1 and ACI-8016-22DWA-AB1; ASC specks and an intermediate cytoplasm staining was observed with ACI-8016-23E5F7-AB1; ASC specks and a strong cytoplasm staining was observed with ACI-8016-23E5F7-AB2; preferential labeling of ASC specks was observed with ACI-8016-2504F3D9-AB 1, ACI-8016-2609F4A9-AB 1, ACI-8016-2617C3A8-A1B, ACI-8016-2622E12F11-AB1 and ACI-8016-2629E8D1-AB1. Few ASC specks and strong cytoplasmic staining was observed with ACI-8016-26A1G2-AB1, ACI-8016-31F10C5-AB1 and A-8016-2617C3A8-AB1. Only weak signal on ASC specks was detected for ACI-8016-2626191D3-AB1.

TABLE 9
Results of immunofluorescence staining with
ASC mAbs in mouse macrophages J774.A1.

| | Localization and relative intensity of staining | | |
|---|---|---|---|
| | | Detection of | Detection of ASC |
| Antibody name | Description | ASC Specks | in cytoplasm |
| ACI-8016-18F4C12-AB1 | ASC speck & cytoplasm | +++ | + |
| ACI-8016-23E5F7-AB1 | ASC speck & cytoplasm | +++ | ++ |
| ACI-8016-23E5F7-AB2 | ASC speck & cytoplasm | +++ | +++ |
| ACI-8016-26A1G2-AB1 | ASC speck & cytoplasm | + | +++ |
| ACI-8016-22D3A6-AB1 | ASC speck & cytoplasm | ++ | + |
| ACI-8016-31F10C5-AB1 | ASC speck & cytoplasm | + | +++ |
| ACI-8016-2504F3D9-AB1 | ASC speck | +++ | − |
| ACI-8016-2609F4A9-AB1 | ASC speck | +++ | − |
| ACI-8016-2610H7D3-AB1 | ASC speck & cytoplasm | + | +++ |
| ACI-8016-2617C3A8-AB1 | ASC speck & cytoplasm | + | +++ |
| ACI-8016-2622E12F11-AB1 | ASC speck | ++ | − |
| ACI-8016-2626B9D3-AB1 | ASC speck | +/− | − |
| ACI-8016-2629E8D1-AB1 | ASC speck | +++ | − |

−, absent; +/−, very weak; +, weak; ++, medium; +++, abundant

### Example 7. Functional Antibody Characterization in Recombinant ASC Polymerization Assay In Vitro

### Methods

### Human Recombinant ASC Polymerization Assay

hASC and hASC-PYD labeled with fluorescent dye DyLight Fluor 488 were recombinantly produced in E. coli, aliquoted and stored at −80° C. Proteins were produced at pH 3.7 to keep them in a monomeric form as they quickly polymerize at pH 7. For each experiment, aliquots of hASC and hASC-PYD-488 were thawed on ice and centrifuged for 15 min at 20000 g. Polymerization assay were implemented from Sborgi et al. (2018). hASC and hASC-PYD-488 were diluted in glycine buffer (50 mM glycine pH 3.7, 150 nM NaCl) to a final concentration of 200 nM and 1.3 nM respectively. A Triton-X100 containing buffer (10% Triton X-100 solution in 50 mM glycine, pH 3.7, 150 mM NaCl) was added to the mix to get a 0.5% final concentration. Polymerization was induced by a rapid change of pH by addition of 1 volume of assay buffer (HEPES 25 mM, NaCl 150 mM, pH 7.4) in 384 well plate with 50 μL final volume per well. Immediately after 10 l of either ASC mAbs, isotype control both at an equimolar ratio diluted in DPBS or 5 M NaCl solution were added. Polymerization was monitored by fluorescence polarization (FP) measurement using filters at 480 nm and 535 nm every 30 sec over 150 min in technical triplicates. Assessment of inhibition was done qualitatively by comparing the curves obtained with equimolar amount of ASC mAbs to the isotype control (maximal polymerization) and NaCl 5M (completely prevents polymerization). To normalize the curves, the first data point was deduced from itself (and all the points) and we added 30 as an arbitrary unit as a starting point for all the curves in order to have a common starting point. For mAbs showing equivalent inhibition to NaCl 5M, an experiment using a serial dilution (1/3) starting at equimolar ratio was performed to extract an IC₅₀. AUC of the whole curves (0 to 150 min) for each dilution of the mAbs was measured in GraphPad. IC₅₀ were extrapolated by plotting AUC measurement vs log 10 of concentration in a nonlinear regression of a dose-response curve. To maintain equivalent antibody amount throughout the serial dilutions, antibodies concentrations were adjusted using the isotype control mAb.

### Mouse Recombinant ASC Polymerization Assay

mASC and mASC-PYD labeled with fluorescent dye DyLight Fluor 488 were recombinantly produced in E. coli, aliquoted and stored at −80° C. Proteins were produced at pH 3.7 to keep them in a monomeric form as they quickly polymerize at pH 7. For each experiment, aliquots of mASC and mASC-PYD-488 were thawed on ice and centrifuge for 15 min at 20,000 g. Polymerization assay were implemented from (Sborgi et al. (2018)). mASC and mASC-PYD-488 were diluted in glycine buffer (50 mM glycine pH 3.7, 150 nM NaCl) to a final concentration of 600 nM and 20 nM respectively. A Triton-X100 containing buffer (10% Triton X-100 solution in 50 mM glycine, pH 3.7, 150 mM NaCl) was added to the mix to get a 0.5% final concentration. Polymerization was induced by addition of 1 volume of assay buffer (HEPES 25 mM, NaCl 150 mM, pH 7.4) in 384 well plate with 50 μL final volume per well. Immediately after 10 μl of either ASC mAbs, isotype control at an equimolar ratio diluted in DPBS or 5 M NaCl solution were added. Aggregation was monitored by FP measurement using filters at 480 nm and 535 nm every 30 sec over 3 h in technical triplicates. Assessment of inhibition was done qualitatively by comparing the curves obtained with equimolar amount of ASC mAbs to the isotype control (maximal polymerization) and NaCl 5M (no polymerization). To normalize the curves, the first data point was deduced from itself (and all the points) and we added 30 as an arbitrary unit as a starting point for all the curves in order to have a common starting point. For mAbs showing equivalent inhibition to NaCl 5 M, an experiment using a serial dilution (1/3) starting at equimolar ratio was performed to extract an IC₅₀. AUC of the whole curves (0 to 150 min) for each dilution of the mAbs is measured in GraphPad. IC₅₀ were extrapolated by plotting AUC measurement vs log 10 of concentration in a nonlinear regression of a dose-response curve. To maintain equivalent antibody amount throughout the serial dilutions, antibodies concentrations were adjusted using the isotype control mAb.

### Results

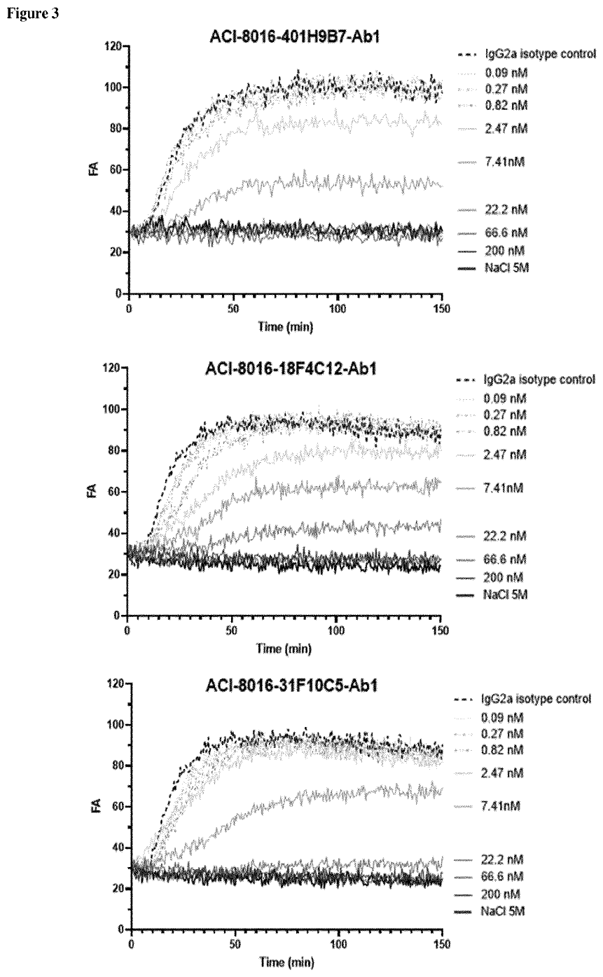

ASC polymerization in the presence of equimolar amount of mAbs was monitored by FP. Different profiles were obtained for different mAbs and representative curves are shown in FIG. 3. Qualitative results are summarized in Table 10.

Complete or almost complete inhibition (strong inhibition) of human ASC polymerization was observed in the presence of ACI-8016-401H9B7-AB1, ACI-8016-18F4C12-AB1, ACI-8016-23E5F7-AB1, ACI-8016-23E5F7-AB2, ACI-8016-26A1G2-AB1, ACI-8016-22D3A6-AB1, ACI-8016-31F10C5-AB1, ACI-8016-2609F4A9-AB1, ACI-8016-2610H7D3-AB1, ACI-8016-2617C3A8-AB1, ACI-8016-2622E12F11-AB1, ACI-8016-2626B9D3-AB1 and ACI-8016-2629E8D1-AB1. Moderate inhibition was seen for ACI-8016-421B10C12D2-AB1, ACI-8016-417E12A8-AB1, ACI-8016-413G10A5-AB1, ACI-8016-407E10A9-AB1, ACI-8016-2504F3D9-AB, and ACI-8016-203G8B10-AB1. Mild inhibition was observed for ACI-8016-416E6G4-AB1, ACI-8016-402H11C9-Ab1, ACI-8016-203B12C3-AB1.

Complete or almost complete inhibition (strong inhibition) of mouse ASC polymerization was observed in the presence of ACI-8016-23E5F7-AB1, ACI-8016-23E5F7-AB2, ACI-8016-26A1G2-AB1, ACI-8016-22D3A6-AB1, ACI-8016-2610H7D3-AB1, ACI-8016-2617C3A8-AB1, ACI-8016-2626B9D3-AB1 and ACI-8016-31F10C5-AB1. Very low to low inhibition is seen ACI-8016-416E6G4-AB1, ACI-8016-203B12C3-AB1, ACI-8016-421B10C12D2-AB1 and ACI-8016-2504F3D9-AB1. Mild inhibition was seen for ACI-8016-2622E12F11-AB1 and ACI-8016-2629E8D1-AB1. No inhibition was seen for ACI-8016-402H11C9-Ab1, ACI-8016-417E12A8-AB1, ACI-8016-413G10A5-AB1, ACI-8016-407E10A9-AB1, ACI-8016-203G8B10-AB1 and ACI-8016-401H9B7-AB1.

TABLE 10
Qualitative assessment of human and mouse ASC polymerization
inhibition in the presence of ASC mAbs.

| | Human ASC | Mouse ASC |
|---|---|---|
| | polymerization | polymerization |
| ASC mAb code | assay | assay |
| ACI-8016-416E6G4-AB1 | + | + |
| ACI-6404_rmG2a_001 | + | + |
| ACI-8016-402H11C9-Ab1 | ++ | − |
| ACI-8016-203B12C3-AB1 | ++ | + |
| ACI-8016-421B10C12D2-AB1 | +++ | ± |
| ACI-8016-417E12A8-AB1 | +++ | − |
| ACI-8016-413G10A5-AB1 | +++ | − |
| ACI-8016-407E10A9-AB1 | +++ | − |
| ACI-8016-203G8B10-AB1 | +++ | − |
| ACI-8016-401H9B7-AB1 | ++++ | − |
| ACI-8016-18F4C12-AB1 | ++++ | + |
| ACI-8016-23E5F7-AB1 | ++++ | + |
| ACI-8016-23E5F7-AB2 | ++++ | + |
| ACI-8016-26A1G2-AB1 | ++++ | + |
| ACI-8016-22D3A6- | ++++ | + |
| AB1_rmG2a_001 | | |
| ACI-8016-31F10C5-AB1 | ++++ | + |
| ACI-8016-2504F3D9-AB1 | +++ | + |
| ACI-8016-2609F4A9-AB1 | ++++ | ++ |
| ACI-8016-2610H7D3-AB1 | ++++ | ++++ |
| ACI-8016-2617C3A8-AB1 | ++++ | ++++ |
| ACI-8016-2622E12F11-AB1 | ++++ | ++ |
| ACI-8016-2626B9D3-AB1 | ++++ | ++++ |
| ACI-8016-2629E8D1-AB1 | ++++ | ++ |

−: no inhibition/equivalent to control IgG2a; ± very low inhibition; + weak inhibition; ++ mild inhibition; +++ moderate inhibition; ++++ strong inhibition.

mAbs showing inhibition of both human and mouse ASC polymerization were further assessed for IC₅₀ determination. The results for efficacy of inhibition of polymerization of human and mouse ASC are summarized Table 11.

TABLE 11
mAb efficacy for human and mouse
ASC polymerization inhibition.

| | Human ASC | Mouse ASC |
|---|---|---|
| | polymerization | polymerization |
| ASC mAb code | assay, IC₅₀, nM | assay, IC₅₀, nM |
| ACI-8016-401H9B7-AB1 | 3.1 ± 2.4 | NA |
| ACI-8016-18F4C12-AB1 | 4.7 ± 1.9 | NA |
| ACI-8016-23E5F7-AB1 | 5.8 ± 1.7 | 21 ± 10.4 |
| ACI-8016-23E5F7-AB2 | 3.8 ± 0.9 | 21.6 ± 11 |
| ACI-8016-26A1G2-AB1 | 4.9 ± 0.7 | 24.8 ± 10.4 |
| ACI-8016-32B6C7-AB1 | 12.1 ± 4.4 | NA |
| ACI-8016-22D3A6-AB1 | 4.2 ± 0.5 | 30.1 ± 18.5 |
| ACI-8016-31F10C5-AB1 | 6.3 ± 1.4 | 34.8 ± 26.3 |
| ACI-8016-2609F4A9-AB1 | 32.8 ± 4.2 | NA |
| ACI-8016-2610H7D3-AB1 | 10.1 ± 6.4 | 60.4 ± 23.2 |
| ACI-8016-2617C3A8-AB1 | 6.0 ± 2.1 | 35.7 ± 24.9 |
| ACI-8016-2622E12F11-AB1 | 19.4 ± 7.7 | NA |
| ACI-8016-2626B9D3-AB1 | 4.9 ± 3.5 | 27.3 ± 5.7 |
| ACI-8016-2629E8D1-AB1 | 14.1 ± 7.8 | NA |

Mean IC₅₀ values from n = 2 or n = 3 independent experiments

This study showed that ASC mAbs can inhibit the polymerization of human ASC in a wide range: from moderate to total inhibition at equimolar ratio. Eight selected ASC mAbs, ACI-8016-23E5F7-AB 1, ACI-8016-23E5F7-AB2, ACI-8016-26A1G2-AB1, ACI-8016-22D3A6-AB1, ACI-8016-31F1OC5-AB1, ACI-8016-2610H7D3-AB 1, ACI-8016-2617C3A8-AB 1 and ACI-8016-2626B9D3-AB 1 showed functional efficacy in inhibition of human (IC50 around 5 nM) and mouse (IC50 around 30 nM) recombinant ASC polymerization. Six mAbs ACI-8016-401H91B7-AB1, ACI-8016-18F4C12-AB1, ACI-8016-321B6C7-AB1, ACI-8016-2609F4A9-AB1, ACI-8016-2622E12F11-AB1 and ACI-8016-2629E8D1-AB1 only inhibited human ASC polymerization. These data allow further exploration of ASC mAbs effect in prevention of propagation of inflammasome activation in vivo.

### Example 8. Assessment of Antibody-Driven Uptake of ASC Polymers

### Methods

### Cell-Based Assay of Uptake of ASC Polymers in Human Monocytic Cell Line

Human monocytic cells were seeded at 5×10⁵ cells per well in the 60-inner wells of 96-well culture plate (Vitaris, COR-3595) and differentiated overnight with 10 ng/mL phorbol 12-myristate 13-acetate (PMA; Sigma, P1585) in culture medium (RPMI 1640 (Gibco, 61870-044)) supplemented with 10% heat inactivated fetal bovine serum (FBS; Gibco, 10500-064), 1% penicillin/streptomycin (PS; Gibco, 10378-016). The following day, the medium was replaced by serum free medium (SFM; growth medium without serum) and cells were treated with DPBS (n=3; negative control) or mixture of hASC polymers labeled with pHrodo (see below) preincubated 15 min either with isotype IgG2a control (420 nM, n=3; positive control) or F(ab′)2 fragment (obtained by enzymatic cleavage of ACI-8016-401H9B7-Ab1 (Genovis, FragIT™ Z kit); 350 nM, n=3; positive control) or ASC mAbs (420 nM in triplicate). As negative control for phagocytosis, cells were pretreated with cytochalasin D (Life Technologies) at 1 μM for 30 min before treatment with hASC polymers. Kinetics of uptake of hASC polymers was monitored (images taken every 1 h during 22 h, mean of 4 images per well) and fluorescent signal quantified on the IncuCyte® Zoom Live Cell Analysis System (Sartorius, USA). Qualitative evaluation was done to summarize results of independent experiments.

Preparation of Recombinant hASC Polymers, Labeling with pHrodo and mAb Treatment

Human ASC polymerization was performed as described in Sborgi et al. (2018). In brief, recombinant hASC protein (Selvita) was formulated in acidic buffer composed of 50 mM glycine pH 3.8 and 150 mM NaCl at concentration of 42 μM (1 mg/mL). hASC protein was diluted 5 times (v/v) in glycine formulation buffer and then 2 times (v/v) in assay buffer (25 mM HEPES and 150 mM NaCl pH 7.4) to induce polymerization by a rapid change of pH for 3 h at RT. At the end of polymerization, polymers were centrifuged and transferred to DPBS buffer at concentration of 0.1 mg/mL. pH-sensitive pHrodo dye was conjugated to free amine residues of hASC polymers following the manufacturer instructions (Invivogen, P36013). Monoclonal antibodies were mixed 1:1 molar ratio with hASC polymers and incubated for 15 min at RT before cell treatment (final concentration of mAb and hASC polymers 420 nM).

### Cell-Based Assay of Uptake of ASC Polymers in Mouse Bone Marrow Derived Macrophages

Isolation and differentiation of mouse bone marrow derived macrophages (BMDM) was done as described by Madaan et al (2014). Briefly, ten weeks old Balb/c mouse (Charles River, France) was sacrificed in C02 chamber. After isolation, the femurs were flushed with culture medium (RPMI 1640 (Gibco, 61870-044)) supplemented with 10% heat inactivated fetal bovine serum (FBS; Gibco, 10500-064), 1% penicillin/streptomycin (PS; Gibco, 10378-016) to remove bone marrow. After a mechanical dissociation, red blood cells were lysed (Miltenyi, 130-094-183) to keep only the bone marrow progenitors. These progenitors were differentiated into macrophages for 8 days in vitro in the presence of mouse M-CSF at 100 ng/mL (Peprotech, 315-02) in 100 mm Petri dishes. BMDMs were detached with non-enzymatic solution and seeded at 5×10⁵ cells per well in the 60-inner wells of 96-well culture plate (Vitaris, COR-3595). The following day, the medium was replaced by serum free medium (SFM; growth medium without serum) and cells were treated with DPBS (n=3; negative control) or mixture of mouse ASC polymers labeled with pHrodo (see below) preincubated for 15 min either with isotype IgG2a control (420 nM, n=3; positive control) or ASC mAbs (420 nM in triplicate). As negative control for phagocytosis, cells were pretreated with cytochalasin D (Life Technologies) at 1 μM 30 min before treatment with mASC polymers. Kinetics of uptake of mASC polymers was monitored (images taken every 1 h during 22 h, mean of 4 images per well) and fluorescent signal quantified on the IncuCyte® Zoom Live Cell Analysis System (Sartorius, USA). Qualitative evaluation was done to summarize results of independent experiments.

Preparation of Recombinant mASC Polymers, Labeling with pHrodo and mAb Treatment

Mouse ASC polymerization was performed as described in Sborgi et al. (2018). In brief, recombinant mASC protein (Selvita) was formulated in acidic buffer composed of 50 mM glycine pH 3.8 and 150 mM NaCl at concentration of 44.4 μM (1 mg/mL). mASC protein was diluted 1/5 times (v/v) in glycine formulation buffer and then 2 times (v/v) in assay buffer (25 mM HEPES and 150 mM NaCl pH 7.4) to induce a rapid change in pH jump to start polymerization for 3 h at RT. At the end of polymerization, polymers were centrifuged and transferred to DPBS buffer at concentration of 1 mg/mL. pH-sensitive dye pHrodo was conjugated to free amine residues of ASC polymers following the manufacturer instructions (Invivogen, P36013). Monoclonal antibodies were mixed 1:1 molar ration with mASC polymers and incubated for 15 min at RT before cell treatment (final concentration of mAb 420 nM and mASC polymers 1,68 μM.

### Results

Increase of hASC Polymers Uptake by mAb in Monocytic Cell Line

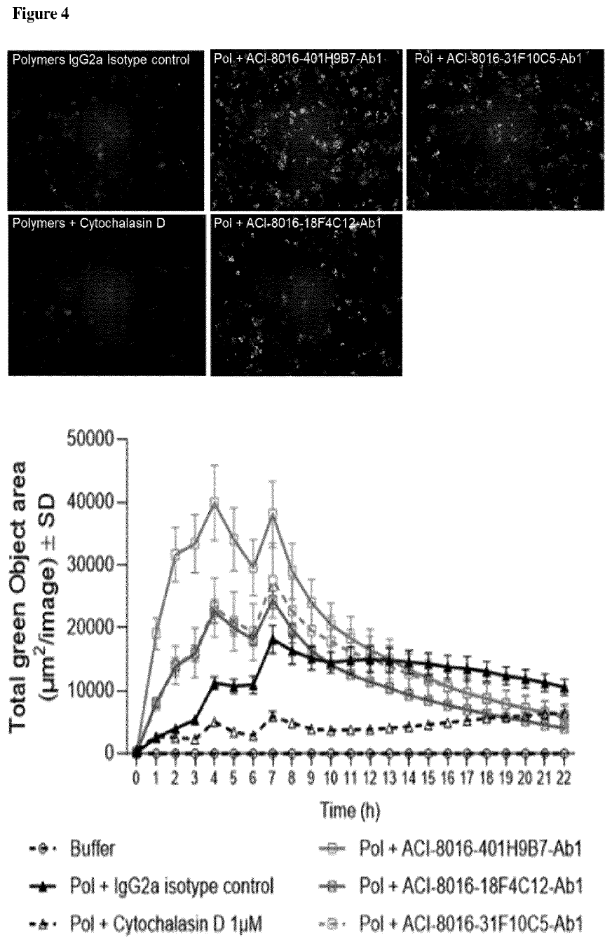

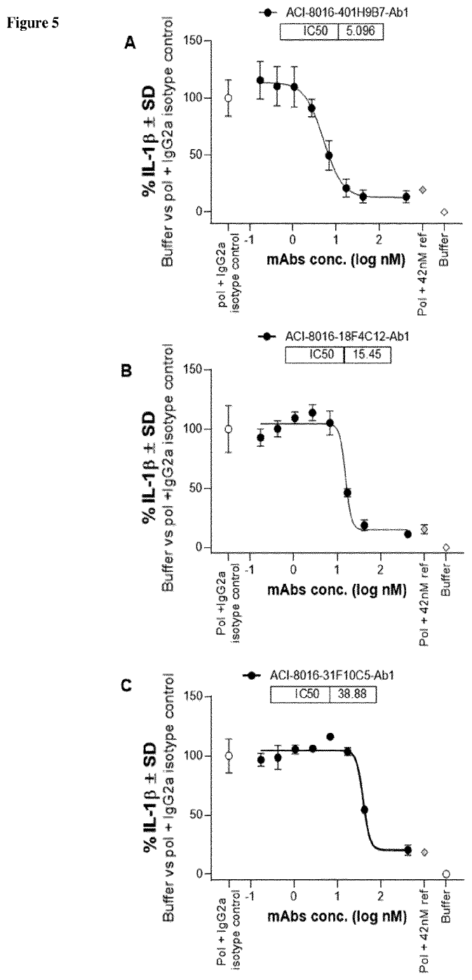

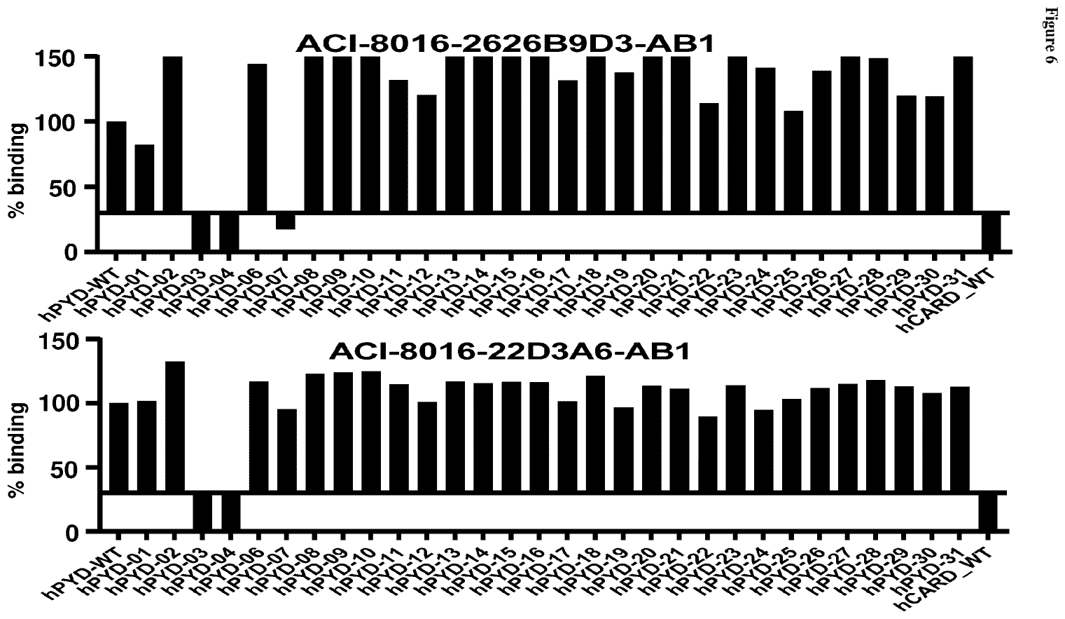

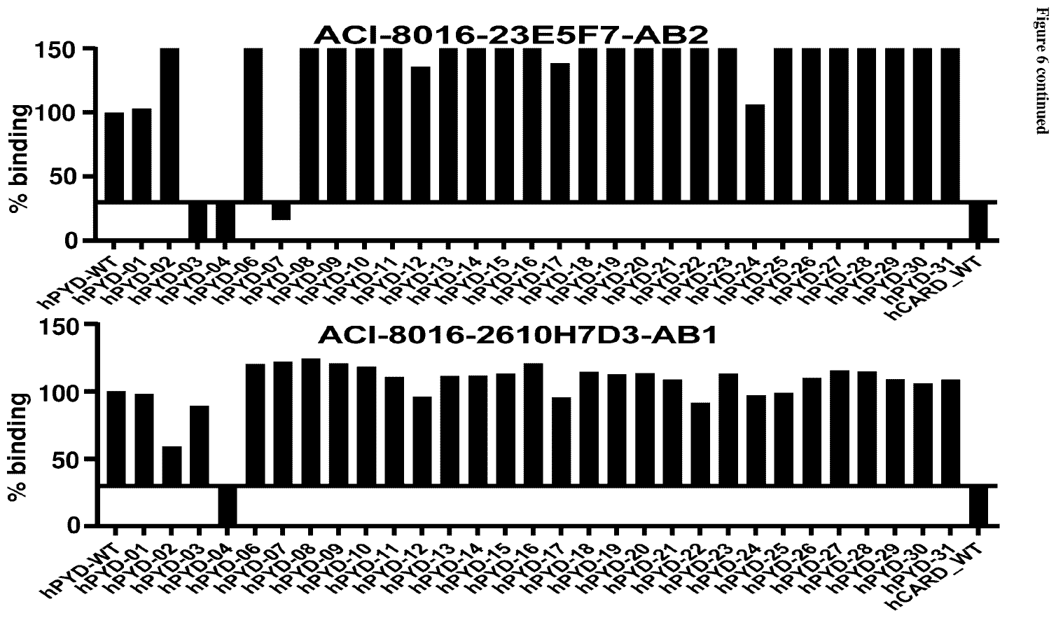

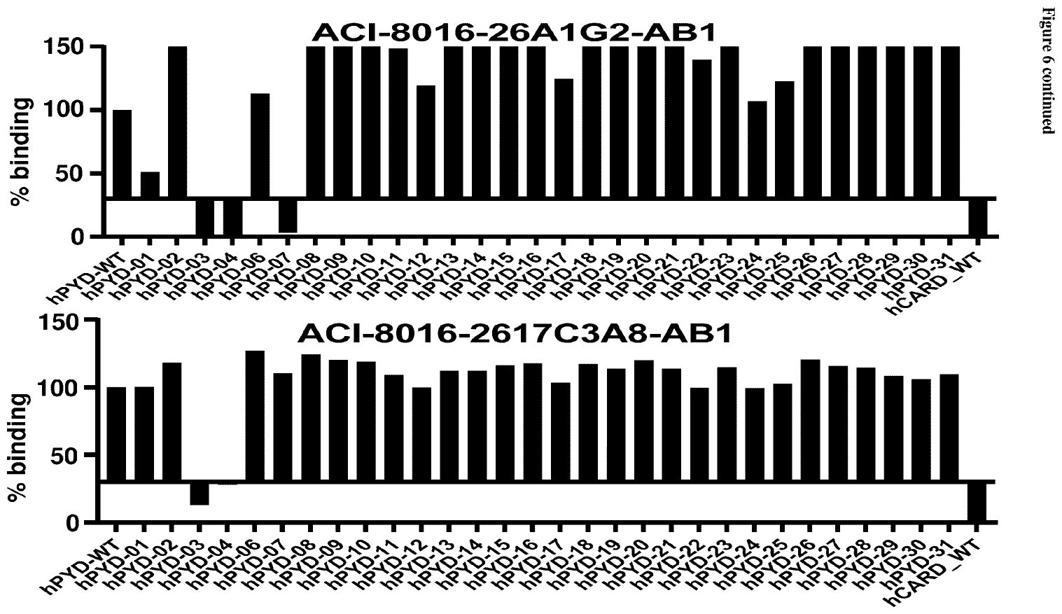

ACI-8016-401H9B7-Ab1, ACI-8016-18F4C12-Ab1 and ACI-8016-31F10C5-Ab1 increased the initial phase of uptake of hASC polymers compared to isotype IgG2a control as monitored by pHrodo fluorescence. After 10 h the levels of fluorescence were similar to isotype IgG2a control in all mAb-treated conditions (FIG. 4). Table 12 summarizes the results of uptake observed at 3 h of treatment compared to isotype IgG2a control. In presence of mAbs, internalization of hASC polymers labeled with pHrodo was increased for all mAbs tested with exception of antibody ACI-8016-401H9B7-Ab1 F(ab′)2 fragment. The level of uptake of labeled hASC polymers was higher at 3h for ACI-8016-401H9B7-Ab1, ACI-8016-18F4C12-Ab1 and ACI-8016-31F10C5-Ab1 compared to the other antibodies.

TABLE 12
Effect of mAbs on uptake of hASC polymers compared to
isotype control in human monocytic cell line at 3h.

| ACI number | Uptake at 3 h |
|---|---|
| ACI-8016-401H9B7-Ab1 | +++ |
| ACI-8016-401H9B7-Ab1 F(ab′)2 | − |
| ACI-8016-18F4C12-Ab1 | +++ |
| ACI-8016-23E5F7-Ab1 | ++ |
| ACI-8016-23E5F7-Ab2 | ++ |
| ACI-8016-26A1G2-Ab1 | ++ |
| ACI-8016-22D3A6-Ab1 | ++ |
| ACI-8016-31F10C5-Ab1 | +++ |

−: <1x increase vs isotype control (no inhibition or equivalent to control IgG2a); ++: from 2x to 3x increase vs isotype control (mild inhibition); +++: >3x increase vs isotype control (moderate inhibition). N = 3-5 independent experiments

Increase of mASC Polymers Uptake by ASC mAbs in BMDMs

In presence of isotype control, no fluorescence was observed inside BMDMs indicating an absence of mASC polymers internalization. In presence of ASC mAbs ACI-8016-23E5F7-Ab1, ACI-8016-26A1G2-Ab1, ACI-8016-22D3A6-Ab1 and ACI-8016-31F10C5-Ab1, uptake was more efficient at 1 h compared to ACI-8016-18F4C12-Ab1.

Table 13 summarizes the results of uptake observed at 1 h of treatment compared to levels of isotype IgG2a control.

TABLE 13
Effect of mAbs on uptake of mASC polymers compared
to isotype IgG2a control in mouse BMDMs at 1h.

| ACI number | Uptake at 1 h |
|---|---|
| ACI-8016-18F4C12-Ab1 | ++ |
| ACI-8016-23E5F7-Ab1* | +++ |
| ACI-8016-26A1G2-Ab1 | +++ |
| ACI-8016-22D3A6-Ab1 | +++ |
| ACI-8016-31F10C5-Ab1 | +++ |

++: from 7x to 15x increase vs isotype control (mild inhibition); +++: >15x increase Vs isotype control (moderate inhibition). n=3 independent experiments;
*n = 1

The capacity of ASC mAbs to increase the uptake of ASC polymers will be beneficial in vivo to increase the uptake of extracellular ASC specks and to prevent the propagation of ASC-dependent inflammation by directing the ASC speck toward the degradation pathway.

### Example 9. Functional Antibody Assessment in Cell-Based Inflammasome Propagation Assays

### Methods

Cell-Based Assay of Propagation of Inflammasome Activation in Human Monocytic Cell Line Human monocytic cells (DSMZ, ACC 16) were seeded at 5×10⁵ cells per well in the 60-inner wells of 96-well culture plate (Vitaris, COR-3595) and differentiated overnight with 10 ng/mL phorbol 12-myristate 13-acetate (PMA; Sigma, P1585) in culture medium (RPMI 1640 (Gibco, 61870-044)) supplemented with 10% heat inactivated fetal bovine serum (FBS; Gibco, 10500-064), 1% penicillin/streptomycin (PS; Gibco, 10378-016)). The following day, the medium was removed and differentiated human monocytic cells were primed with 10 ng/mL LPS from Escherichia coli (Enzo LifeScience, ALX-581-013-L002) for 3 h at 37° C., 5% CO₂. At the end of priming treatment, the medium was replaced by serum free medium (SFM; growth medium without serum) and cells were treated with polymerization buffer (n=3; negative control) or mixture of ASC polymers (see below) preincubated 15 min either with isotype control IgG2a (at 420 nM, n=6; positive control) or reference ASC mAb ACI-8016-401H9B7-AB1 (fixed concentration of 42 nM and 8 concentrations in triplicate, concentration range 0.2 nM-420 nM) or ASC mAbs (8 concentrations in triplicate, concentration range 0.2 nM-420 nM) as described below. After 24 hours, 30 μL of supernatant were collected and IL-1β was quantified using AlphaLISA technology (PerkinElmer, AL220F) following the manufacturer instructions (quick protocol in 384-well plate) and read fluorescence was detected with EnVision Alpha multiplate reader. Resulting IL-1β concentrations (μg/mL) were normalized (0% correspond to polymerization buffer and 100% to ASC polymers preincubated with isotype control IgG2a) and C₅₀ values were retrieved in software Graph Pad Prism® by using nonlinear regression curve fit model after plotting compound concentrations in log 10 in X-axis and IL-1β concentrations in Y-axis. In addition, the percent inhibition of estimated bottom plateau was extracted to evaluate the maximum inhibitory capacity of the antibodies. In case of incomplete inhibition without bottom plateau, the IC₅₀ values were not determined (ND). Experiments were validated if 1) IL-1β release (positive control) was higher than 7-fold compared to negative control (buffer), 2) inhibition by ACI-8016-401H9B7-AB1 at 42 nM was equal or higher than 60% of the positive control and 3) IC₅₀ of ACI-8016-401H9B7-AB1 was below 10 nM.
Preparation of Recombinant hASC Polymers and mAb Treatment

ASC polymerization was performed as described in Sborgi et al. (2018). In brief, recombinant hASC protein (Selvita) was formulated in acidic buffer composed of 50 mM glycine pH 3.8 and 150 mM NaCl at concentration of 42 μM (1 mg/mL). The day of cell treatment, the hASC protein was diluted 5 times (v/v) in glycine formulation buffer and then 2 times (v/v) in assay buffer (25 mM HEPES and 150 mM NaCl pH 7.4) to induce pH jump and to start polymerization for 3 h at RT.

Monoclonal antibodies were tested in a range of concentrations. Total amount of protein was maintained by adding the isotype control IgG2a mAb to keep molarity of 420 nM equivalent to hASC molarity. After preparation of serial dilutions, mAbs were mixed 1:1 with hASC polymers and incubated for 15 min at RT before cell treatment (final concentration range from 420 nM to 0.2 nM on cells).

### Cell-Based Assay of Propagation of Inflammasome Activation in Mouse Primary Microglia

Cortices isolated from CD1 mice (Charles River, France) at post-natal day 5 were dissociated enzymatically and mechanically as described in Neural Tissue Dissociation Kits (P) (Miltenyi, 130-092-628). From the cell suspension obtained, microglia cells were purified using CD11b/c microbeads as per manufacturer instructions (Miltenyi, 130-093-634). Microglia was plated at a density of 3×10⁵ cells per well onto 60 inner wells of a 96-well tissue culture plates (Sarstedt, 83.3924) and maintained in complete growth medium adapted from Bohlen et al (2017). Growth medium was composed of DMEM/F12 (Gibco, 31331-093) supplemented with 2.5% heat inactivated FBS, 1% PS, 200 ng/mL Tumor growth factor $2 (TGF-02; Peprotech, 100-35B), 100 ng/mL mouse Interleukin-34 (IL-34; R&D Systems, 5195-ML/CF), 5 μg/ml N-acetyl cysteine (Sigma, A9165), 5 μg/ml insulin (Sigma, I6634), 100 μg/mL apotransferrin (Sigma, T1147), 100 ng/mL sodium selenite (Sigma, S-5261) and ovine wool cholesterol (1.5 μg/mL, Avanti Polar Lipids). At Day In Vitro 3 (DIV3), the medium was replaced by serum free medium (SFM; growth medium without serum) and cells were treated with polymerization buffer (n=3; negative control) or mixture of ASC polymers (see below) preincubated 15 min either with isotype control IgG2a (at 420 nM, n=6; positive control) or reference ASC mAb ACI-8016-401H9B7-AB1 (at 420 nM, n=3; negative control) or ASC mAbs (fixed concentration of 420 nM in triplicate). After 24 hours, 30 μL of supernatant were collected and IL-1β was quantified using AlphaLISA technology (PerkinElmer, AL503F) following the manufacturer instructions and fluorescence was detected with EnVision Alpha multiplate reader. Resulting IL-1β concentrations (pg/mL) were normalized (0% correspond to polymerization buffer and 100% to ASC polymers preincubated with isotype control IgG2a).

Preparation of Recombinant mASC Polymers and mAb Treatment

Mouse ASC polymerization was performed as described in Sborgi et al. (2018). In brief, recombinant mASC protein (Selvita) was formulated in acidic buffer composed of 50 mM glycine pH 3.8 and 150 mM NaCl at concentration of 44.4 μM (1 mg/mL). mASC protein was diluted 1.25 times (v/v) in glycine formulation buffer and then 2 times (v/v) in assay buffer (25 mM HEPES and 150 mM NaCl pH 7.4) to induce a rapid change in pH jump to start polymerization for 3 h at RT.

Monoclonal antibodies were mixed 1:1 with mASC polymers and incubated for 15 min at RT before cell treatment (final concentration of mAb 420 nM and mASC polymers 1.68 μM).

### Results

### Inhibition of IL-1β Release in a Human Monocytic Cell Line

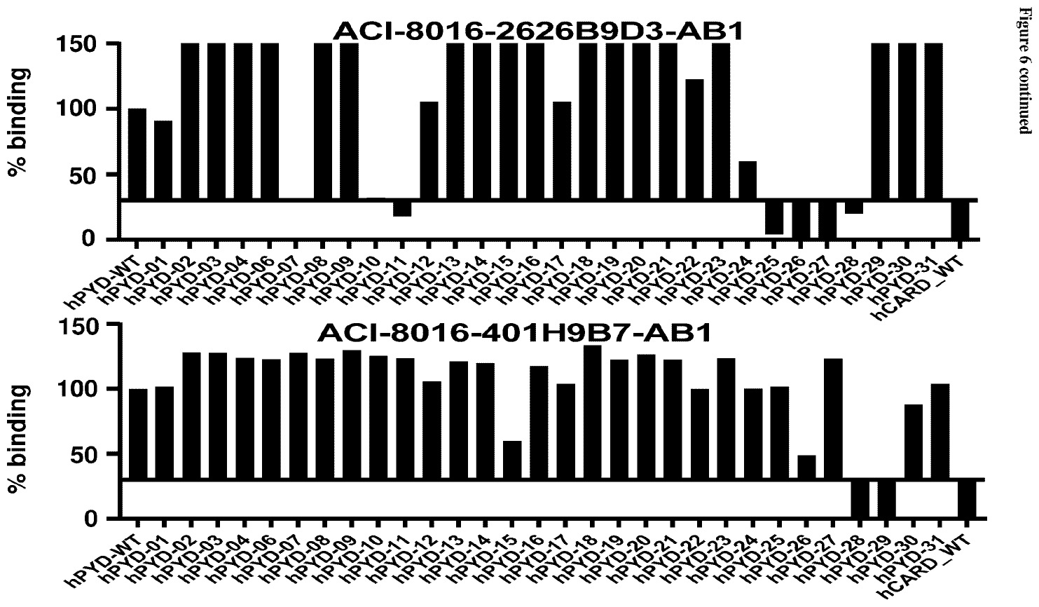

FIG. 5 illustrates representative curves obtained for each mAb. Pre-incubation of hASC polymers with ACI-8016-401H9B7-AB1 (internal reference mAb) at 42 nM inhibited IL-1β release compared to hASC polymers preincubated with isotype control IgG2a in all experiments. In presence of ACI-8016-401H9B7-AB1, IL-1β release was inhibited with an IC₅₀ of 6.94 nM with a bottom plateau at approximatively 80% of inhibition. Table 14 summarizes the results for all tested mAbs.

TABLE 14
Summary of mAb efficacy in inhibition of propagation
of inflammation human monocytic cell line

| | IC50 (nM) | Number of |
|---|---|---|
| Antibody name | Mean ± SD | experiments |
| ACI-8016-401H9B7-AB1 | 6.94 ± 2.69 | 10 |
| ACI-8016-18F4C12-AB1 | 15.88 ± 0.97 | 3 |
| ACI-8016-23E5F7-AB2 | ND | 5 |
| ACI-8016-23E5F7-AB1 | ND | 5 |
| ACI-8016-26A1G2-AB1 | ND | 6 |
| ACI-8016-32B6C7-AB1 | 13.95 ± 0.66 | 2 |
| ACI-8016-22D3A6-AB1 | 41.12 ± 13.15 | 4 |
| ACI-8016-31F10C5-AB1 | 32.99 ± 6.38 | 3 |
| ACI-8016-2504F3D9-AB1 | 16.72 ± 8.63 | 2 |
| ACI-8016-2609F4A9-AB1 | 16.25 ± 0.74 | 2 |
| ACI-8016-2610H7D3-AB1 | 25.17 ± 18.97 | 3 |
| ACI-8016-2617C3A8-AB1 | 20.26 ± 6.56 | 2 |
| ACI-8016-2622E12F11-AB1 | 30.84 ± 22.33 | 3 |
| ACI-8016-2626B9D3-AB1 | 21.11 ± 13.25 | 3 |
| ACI-8016-2629E8D1-AB1 | 14.87 ± 0.76 | 2 |

ND: IC50 was not determined

Out of 15 ASC mAbs tested in this study the most efficient ones to inhibit IL-1β release by human macrophages induced by hASC polymers were ACI-8016-401H9B7-AB1 with an IC50 of 7 nM, ACI-8016-18F4C12-AB1, ACI-8016-32B6C7-AB1, ACI-8016-2629E8D1-AB1, ACI-8016-2504F3D9-AB1 and ACI-8016-2609F4A9-AB1 with IC50s between 14 nM and 17 nM, ACI-8016-2617C3A8-AB1, ACI-8016-2610H7D3-AB1 and ACI-8016-2626B9D3-AB1 with IC50s between 20 nM and 25 nM, ACI-8016-2622E12F11-AB1 and ACI-8016-31F10C5-AB1 with IC50s between 31 nM and 33 nM and ACI-8016-22D3A6-AB1 with an IC50 at 41 nM. Although ACI-8016-23E5F7-AB1, ACI-8016-23E5F7-AB2 and ACI-8016-26A1G2-AB1 showed some inhibition, IC50 values were not determined.

Inhibition of IL-1β Release in Mouse Primary Microglial Cells

The results of IL-1b release triggered by ASC polymers preincubated with ASC mAbs are summarized in Table 15. The reference mAb ACI-8016-401H9B7-AB1-which is specific towards the human ASC sequence does not inhibit IL-1β release in mouse cells, as expected. Out of 15 mAbs tested in this assay, the most efficient in inhibiting IL-1β release were ACI-8016-18F4C12-AB1, ACI-8016-31F10C5-AB1, ACI-8016-2504F3D9-AB1, ACI-8016-2609F4A9-AB1, ACI-8016-2610H7D3-AB1, ACI-8016-2617C3A8-AB1, ACI-8016-2622E12F11-AB1, and ACI-8016-2629E8D1-AB1 with more than 80% inhibition. ACI-8016-23E5F7-AB1, ACI-8016-23E5F7-AB2, and ACI-8016-32B6C7-AB1 decreased IL-1p release in arrange between 80% and 60%. ACI-8016-26A1G2-AB1, ACI-8016-22D3A6-AB1, and ACI-8016-2626B9D3-AB1 showed reduction of IL-1β release in a range between 40% and 60%.

TABLE 15
Summary of mAb efficacy in inhibition of propagation
of inflammation in mouse primary microglial cells

| | % IL-1β inhibition | Number of |
|---|---|---|
| Antibody name | (average ± SD) | experiments |
| ACI-8016-18F4C12-AB1 | 82.78 ± 12.59 | 6 |
| ACI-8016-23E5F7-AB1 | 69.40 ± 19.19 | 5 |
| ACI-8016-23E5F7-AB2 | 73.31 ± 18.25 | 2 |
| ACI-8016-26A1G2-AB1 | 52.14 ± 32.37 | 3 |
| ACI-8016-32B6C7-AB1 | 66.74 ± 19.61 | 3 |
| ACI-8016-22D3A6-AB1 | 40.05 ± 30.90 | 3 |
| ACI-8016-31F10C5-AB1 | 90.48 ± 10.07 | 4 |
| ACI-8016-2504F3D9-AB1 | 92.73 ± 11.50 | 3 |
| ACI-8016-2609F4A9-AB1 | 82.33 ± 8.66 | 3 |
| ACI-8016-2610H7D3-AB1 | 87.35 ± 13.11 | 5 |
| ACI-8016-2617C3A8-AB1 | 94.29 ± 7.81 | 3 |
| ACI-8016-2622E12F11-AB1 | 95.91 ± 9.20 | 5 |
| ACI-8016-2626B9D3-AB1 | 52.04 ± 13.77 | 3 |
| ACI-8016-2629E8D1-AB1 | 88.77 ± 6.08 | 3 |

Values define the percentage of IL-1β inhibition normalized to the isotype control mAb

### Example 10: Antibody Sequencing

Clonal hybridoma cell lysates were used for gene sequencing of the variable region. Mouse hybridomas were harvested and lysed using a lysis buffer containing guanidinium salts to deactivate RNases. cDNA was obtained by reverse transcription of total mRNA. DNA fragment coding for antibody variable region were amplified by RACE-PCR (Takara Bio, cat #634839) using specific primer annealing in the antibody constant region. PCR products were gel purified and cloned into shuttle vector for Sanger sequencing. Genomic DNA was then eliminated by RNase-free DNase, and RNA was purified with a silica-based affinity column using multiple washes and eluted from the column using RNase-free water. Once the RNA was extracted, its purity and concentration was measured spectrophotometrically. The integrity of the RNA was assessed on a denaturing agarose gel and RNA was reverse transcribed into cDNA using reverse transcriptase (RT). Before adding the RT reaction mixture, the RNA was heated to 70° C. for 10 min in order to disrupt RNA secondary structures. The RT products were directly used for PCR amplification. For high-fidelity PCR amplification of the cDNA, each of the variable region primers corresponding to the different gene families encoding for antibodies were individually mixed with the constant primer, for VH and VL separately in a total reaction volume of 50 μl. Initially, a degenerate primer pool was used (12 for VH and 12 for VL) and, depending on the results, a second pool was used to obtain PCR products. After the PCR reaction, the products were analyzed by gel electrophoresis on 2% agarose gels stained with ethidium bromide. The PCR products for VL and VH were individually purified on an agarose gel using tris-acetate-EDTA (TAE). The purified fragments excised from the gel were sequenced using the dye-terminator sequencing method using the same primers as those used for PCR. Sequencing was carried out in both directions to provide overlap at both ends. The sequences were analyzed using multiple sequence alignment (Clustal tool) and annotated using the algorithm of Kabat as described in Kabat et al., Sequences of Proteins of Immunological Interest, 91-3242 (1991). Nucleotide sequences of the heavy chain and light chain variable domains (VH and VL) are shown in Table 16. Translated protein sequences for selected heavy (VH) and light (VL) chain variable domains, and their complementarity-determining regions (CDRs) are shown in Table 17.

TABLE 16
Nucleotide sequences of the Heavy Chain and Light Chain Variable Domains (VH and VL)

| | Antibody | | |
|---|---|---|---|
| Hybridoma | Name | VH sequence | VL sequence |
| 416E6G4 | ACI-8016- | CAGGTGCAGCTGAAGCAGTC | GACATTGTGCTGACCCAATC |
| | 416E6G4- | AGGACCTGGCCTAGTGCAGC | TCCAGCTTCTTTGGCTGTGT |
| | AB1 | CCTCACAGAGCCTGTCCATC | CTCTAGGGCAGAGGGCCACC |
| | | ACCTGCACAGTCTCCGGCAT | ATCTCCTGCAGAGCCAGCGA |
| | | CTCATTATCTAGCTATGGTC | AAGTGTTGATAATTATGGCA |
| | | TACATTGGGTTCGCCAGTCT | TAAGTTTTATAAACTGGTTC |
| | | CCAGGAAAGGGTCTGGAGTG | CAACAGAAACCAGGACAGCC |
| | | GCTGGGAGTGATATGGAGTG | ACCCAAACTCATCATCTATG |
| | | GTGGAAACACAGACTATAGT | CTACATCCAATCAAGGATCC |
| | | GCAACTTTCATATCCAGACT | GGGGTCCCTGCCAGATTTAG |
| | | GAGCATCAGCAAGGACAATT | TGGCAGTGGGTCTGGGACAG |
| | | CCAAGAGCCAAGTTTTCTTT | ACTTCAGCCTCAACATCCAT |
| | | AAAATGGACAGTCTGCAAGT | CCTATGGAGGAGGATGATAC |
| | | TGATGACACAGCCATATATT | TGCAATGTATTTCTGTCAAC |
| | | ACTGTGCCAGAAATGAGGTC | AAAGTAAGGAGGTTCCGTGG |
| | | TGGGGTCAAGGAACCTCAGT | ACGTTCGGTGGAGGCACCAA |
| | | CACCGTCTCTTCA | GCTGGAAATCAAA |
| | | (SEQ ID NO: 18) | (SEQ ID NO: 19) |
| 402H11C9 | ACI-8016- | CAGGTCCAGCTGCAGCAGTC | GATATTGTGCTAACTCAGTC |
| | 402H11C9- | TGGGGCTGAACTGGCAAGAC | TCCAGCCACCCTGTCTGTGA |
| | Ab1 | CTGGGGCCTCAGTGAAGATG | CTCCAGGAGATAGCGTCAGT |
| | | TCCTGCAAGGCTTCTGGCTA | CTTTCCTGCAGGGCCAGCCA |
| | | CACCTTTACTACCTACACGA | AAGTATTGGCAACAACCTAC |
| | | TGCACTGGGTAAAACAGAGG | ACTGGTATCAACATAAATCA |
| | | CCTGGACAGGGTCTGGAATG | CATGAGTCTCCAAGGCTTCT |
| | | GATTGGATACATTAATCCTA | CATCAAGTATGCTTCCCAGT |
| | | GCAGTGGTTATACTTATTAC | CCATCTCTGGGATCCCCTCC |
| | | AATCAGAACTTCAAGGACAA | AGGTTCAGTGGCAGTGAATC |
| | | GGCCACATTGACTGCAGACA | AGGGACAGATTTCACTCTCA |
| | | AATCCTCCAGCACAGCCTAC | GTATCAACAGTGTGGAGACT |
| | | ATGCAACTGAGCAACCTGGC | GAAGATTTTGGAATCTATTT |
| | | ATCTGACGACTCTGCAGTCT | CTGTCAACAGAGTAAGAGCT |
| | | ATTACTGTGCTCTAGGGGGT | GGCCGTGGACGTTCGGTGGA |
| | | AACCCGGCCTGGTTTGCTTA | GGCACCAAGCTGGAAATCAA |
| | | CTGGGGCCAGGGGACTCTGG | A |
| | | TCACTGTCTCTGCA | (SEQ ID NO: 29) |
| | | (SEQ ID NO: 28) | |
| 203B12C3 | ACI-8016- | CAGGTGCAGCTGAAGCAGTC | GATATCCAGATGACACAGAC |
| | 203B12C3- | AGGACCTGGCCTAGTGCAGC | TTCATCCTCCCTGTCTGCCT |
| | AB1 | CCTCACAGAGCCTGTCCATC | CTCTGGGAGACAGAGTCACC |
| | | ACCTGCAAAGTCTCTGACTT | ATCAGTTGCAGGGCAAGTCA |
| | | CTCATTAAGTCACTATGGTG | GGACATTAGCAATTATTTAA |
| | | TACATTGGGTTCGCCAGTCT | ACTGGTATCAGCAGAGACCG |
| | | CCAGGAAAGGGTCTGGAGTG | GATGGAACTGTTAAACTCCT |
| | | GCTGGGAGTGATATGGAGTG | GATCTACTACACATCGAAGG |
| | | GTGGAACCACAGAGTATAAT | TGCACTCAGGAGTCCCATCA |
| | | GAAGCTTTCATTTCCAGACT | AGGTTCAGTGGCAGTGGGTC |
| | | GACCATCAGCAAGGACAATT | TGGAACAGATTTTTCTCTCA |
| | | CCAAGAGCCAAATTCTCTTT | CCATTAGCAACCTGGAGCAA |
| | | AGAATGAACAGTCTGCAACC | GAAGATTTTGCCACTTACTA |
| | | TGCTGACACAGCCATATATT | TTGCCAACAGGGTAATACGC |
| | | ACTGTGCCAGAAAAGGGGAT | GTCCGTTCACGTTCGGAGGG |
| | | TTCTACGGTGACTATTTTGA | GGGACCAAGCTGGAAATAAA |
| | | CTACTGGGGCCAAGGTACCA | A |
| | | CTCTCACAGTCTCCTCA | (SEQ ID NO: 39) |
| | | (SEQ ID NO: 38) | |
| 421B10 | ACI-8016- | GAGGTTCAGCTGCAGCAGTC | GACATTGTGATGTCACAGTC |
| C12D2 | 421B10 | TGGGGCAGACCTTGTGAAGC | TCCTTCCTCCCTGGCTGTGT |
| | C12D2- | CAGGGGCCTCAGTCAAATTG | CAGCAGGAGAGAAGGTCACT |
| | AB1 | TCCTGCACAGCTTCTGGCTT | ATGAGCTGCCAATCCAGTCA |
| | | CAACATTGAAGACACCTATA | GAGTCTACTCAACGGTAGAA |
| | | TCCACTGGGTGAAACAGAGG | CCCGGAGGAACTACTTGGCT |
| | | CCTGCACGGGGCCTGGAGTG | TGGTACCAGCAGAAACCAGG |
| | | GGTTGGAAGGATTGATCCTG | GCAGTCTCCTAAACTGCTGA |
| | | CGAATGGTAATACTGATTAT | TCTACTGGGCATCCACTCGG |
| | | GACCCGAAGTTCCAGGGCAA | AAATCTGGGGTCCCTGATCG |
| | | GGCCACTATGATAACAGACA | CTTCACAGGCAGTGGATCTG |
| | | CATCCTCCAACACAGTCTAC | GGACAGATTTCACTCTCACC |
| | | CTGCAGGTCAGCAGCCTGAC | ATCACCAGTGTGCAGGCTGA |
| | | ATCTGAGGACACTGCCGTCT | AGACCTGGCAGTTTATTACT |
| | | ATTTCTGTGGTAGACACGAC | GCAAGCAATCTTATAATCTG |
| | | GGGTATGATATGGACTACTG | GTCACGTTCGGTGCTGGGAC |
| | | GGGTCAAGGAACCTCAGTCA | CAAGCTGGAACTGAAA |
| | | CCGTCTCCTCA | (SEQ ID NO: 49) |
| | | (SEQ ID NO: 48) | |
| 417E12A8 | ACI-8016- | CAGGTCCAACTGCAGCAGCC | GACATTGTGATGACCCAGTC |
| | 417E12A8- | TGGGGCTGAGCTGGTGAGGC | TCAAAAATTCATGTCCACAT |
| | AB1 | CTGGGGCTTCAGTGATACTG | CAGTAGGAGACAGGGTCACC |
| | | TCCTGCAAGGCTTCTGGCTA | GTCACCTGCAAGGCCAGTCA |
| | | CACCTTCACCAGCTCCTGGA | GAATGTGGGTACTAATGTAG |
| | | TTACCTGGGTGAAACAGAGG | CCTGGTATCAACAGAGACCA |
| | | CCTGGACAAGGCCTTGAGTG | GGGCAATCTCCTAAACCACT |
| | | GATCGGGAATATTTATCCTT | GATTTACTCGGCATCCTACC |
| | | CTGATAGTTATACTAACTCC | GGAACAGTGGAGTCCCTGAT |
| | | AATCAAAAGTTCAAGGACAA | CGCTTCACAGGCAGTGGATC |
| | | GGCCACATTGACTGTAGACA | TGGGACAGATTTCACTCTCA |
| | | AATCTTCCAGCACAGCCTAC | CCATCACCAATGTGCAGTCT |
| | | ATGCACCTCATCAGCCCGAC | GAAGACTTGGCAGAGTATTT |
| | | ATCTGAGGACTCTGCAGTCT | CTGTCAGCAATATAACAACT |
| | | ATTACTGTACAAGAAAAGAC | ATCCTCTCACGTTCGGTGCT |
| | | TTTGTTTACGACGCCTGGTA | GGGACCAAGCTGGAGCTGAA |
| | | CTTCGATGTCTGGGGCGCAG | A |
| | | GGACCACGGTCACCGTCTCC | (SEQ ID NO: 59) |
| | | TCA | |
| | | (SEQ ID NO: 58) | |
| 413G10A5 | ACI-8016- | CAGGTGCAGCTGAAGGAGTC | GATGTTGTGATGACCCAAAC |
| | 413G10A5- | AGGACCTGGCCTGGTGGCGC | TCCACTCTCCCTGCCTGTCA |
| | AB1 | CCTCACAGAGCCTGTCCATC | GTCTTGGAGATCAAGCCTCC |
| | | ACTTGCACTGTCTCTGGGTT | ATCTCTTGCAGATCTAGTCA |
| | | TTCATTAACCAGTTATGGTG | GAGCCTTGTACACAGTAATG |
| | | TACACTGGGTTCGCCAGCCT | GAAACACCTATTTACATTGG |
| | | CCAGGAAAGGGTCTGGAGTG | TACCTGCAGAAGCCAGGCCA |
| | | GCTGGGAGTAATATGGGCTG | GTCTCCAAAGCTCCTGATCT |
| | | GTGGAAGCACAAATTATAAT | ACAAAGTTTCCAACCGATTT |
| | | TCGGCTCTCATGTCCAGACT | TCTGGGGTCCCAGACAGGTT |
| | | GAGCATCAGCAAAGACAACT | CAGTGGCAGTGGATCAGGGA |
| | | CCAAGAGCCAAGTTTTCTTA | CAGATTTCACACTCAAGATC |
| | | AAAATGAACAGTCTGCAAAC | AGCAGAGTGGAGGCTGAGGA |
| | | TGCTGACACAGCCATGTACT | TCTGGGAGTTTATTTCTGCT |
| | | ACTGTGCCAGATGGGACTAC | CTCAAAGTACACATGTTCCG |
| | | GTTAATAGCTACGAGGCTAT | TACACGTTCGGAGGGGGGAC |
| | | GGACTACTGGGGTCAAGGAA | CAAGCTGGAAATAAAA |
| | | CCTCAGTCACCGTCTCCTCA | (SEQ ID NO: 69) |
| | | (SEQ ID NO: 68) | |
| 407E10A9 | ACI-8016- | GAAGTGCAGCTGGTGGAGTC | CAGGCTGTTGTGACTCAGGA |
| | 407E10A9- | TGGGGGAGGCTTCGTGAAGC | ATCTGCACTCACCACATCAC |
| | AB1 | CTGGAGGGTCCCTGAAACTC | CTGGTGAAACAGTCACACTC |
| | | TCCTGTGCAGCCTCTGGATT | ACTTGTCGCTCAAGTACTGG |
| | | CACTTTCAGTAACTATGCCA | GGCTGTTACAACTAATAACT |
| | | TGTCTTGGGTTCGCCAGACT | ATGCCAACTGGGTCCAAGAA |
| | | CCGGAGAAGAGGCTGGAATG | AAACCAGATCATTTTTTCAC |
| | | GGTCGCAACCATTTATAGTG | TGGTCTAATAGGTGGTACCG |
| | | GTGGTATATTCACCTACTAT | ACAACCGACCTCCAGGTGTT |
| | | CCAGACAGTGTGAAGGGTCG | CCTGCCAGATTCTCAGGCTC |
| | | ATTCACCATCTCCAGAGACA | CCTGATTGGAGACAAGGCTG |
| | | GTGCCAAGAACACCCTGTAC | CCCTCACCATCACAGGGGCA |
| | | CTGCAAATGACCAGTCTGAG | CAGACTGAGGATGAGGCAAT |
| | | GTCTGAGGACACGGCCATAT | ATATTTCTGTGCTCTATGGT |
| | | ATTACTGTGCAAGACTTGGG | ACAACAACCATTGGGTGTTC |
| | | GGTTCTTACTGGGGCCAAGG | GGTGGAGGAACCAAACTGAC |
| | | GACTCTGGTCACTGTCTCTG | TGTCCTA |
| | | CA | (SEQ ID NO: 79) |
| | | (SEQ ID NO: 78) | |
| 203G8B10 | ACI-8016- | GAAGTGAAGCTTGAGGAGTC | GATATTGTGATGACGCAGGC |
| | 203G8B10- | TGGAGGAGGCTTGGTGCATC | TGCCTTCTCCAATCCAGTCA |
| | AB1 | CTGGAGGATCCATGAAACTC | CCCTTGGAACATCAGCTTCC |
| | | TCCTGTGTTGCCTCTGGATT | ATCTCCTGCAGGTCTAGTAA |
| | | CACTTTCAGTAACTACTGGA | GACTCTCCTACATAGTGATG |
| | | TGAACTGGGTCCGCCAGTCT | GCTTCACTTATTTGTATTGG |
| | | CCAGAGAAGGGGCTTGAGTG | TATCTGCAGAGGCCAGGCCA |
| | | GGTTGCTCAAATTAGATTGA | GTCTCCTCAACTCCTGATAC |
| | | AATCTGAAAATTATGCGACA | ATCGGGTGTCCAATCTGGCC |
| | | CATTCTGCGGAGTCTGTTAA | TCAGGAGTCCCAAACAGGTT |
| | | AGGGAGGCTCACCATCTCAA | CAGCGGCAGTGAGTCAGGCA |
| | | GAGATGATTCCAAAAGTAGT | CTGATTTCACACTGAGAATC |
| | | GTCTACCTGCAAATGAACAA | AGCAGAGTGGAGCCTGAGGA |
| | | CTTGAGGGCTGAAGACACTG | TGTGGGTGTTTATTACTGTG |
| | | GAATTTATTACTGCTTAGCG | CTCAAATGCTAGAACTCCCG |
| | | GGATGGGGTCTTGACTACTG | CTCACGTTTGGTTCTGGGAC |
| | | GGGCCAAGGCACCACTCTCA | CAAGCTGGAGCTGAAG |
| | | CAGTCTCCTCA | (SEQ ID NO: 89) |
| | | (SEQ ID NO: 88) | |
| 401H9B7 | ACI-8016- | CAGATCCAGTTGGTGCAGTC | GACATCCAGATGACTCAGTC |
| | 401H9B7- | TGGACCTGAGCTGAAGAAGC | TCCAGCCTCCCTATCTGCAA |
| | AB1 | CTGGAGAGACAGTCAAGATC | CTGTGGGAGAAACTGTCACC |
| | | TCCTGCAAGGCTTCTGGATA | ATCACATGTCGAGCAAGTGA |
| | | TACCTTCACAAAGTATGGAA | AAATATTTACACTTATTTAG |
| | | TGAACTGGGTGAAGCAGGCT | CATGGTATCAGCAGAAACAG |
| | | CCAGGAAAGGGTTTAAAGAG | GGAAAATCTCCTCAGCTCCT |
| | | GATGGGCTGGATAAACACCT | GGTCCATAATGCAAACACCT |
| | | ACACTGGAGAGCCAACATAT | TAGTAGAAGGTGTGCCATCA |
| | | TCTGATGACTTCAAGGGACG | AGGTTCAGTGGCAGTGGATC |
| | | ATTTGCCTTTTCTTTGGAAA | AGGCACACAGTTTTCTCTGA |
| | | CCTCTGCCAGCACTGCCTAT | AGATCAACAGCCTAGAGCCT |
| | | TTGCAGATCAACAACCTCAA | GAAGATTTTGGGAATTATTA |
| | | AAATGACGACACGGCTACAT | TTGTCAATATCATTATGGTA |
| | | ATTTCTGTGCAAGACTGGGC | GTCCGTACACGTTCGGAGGG |
| | | TATGCTAATTCCTTTGACTC | GGGACCAAGCTGGAGATAAA |
| | | CTGGGGCCAAGGCACCACTC | G |
| | | TCACAGTCTCCTCC | (SEQ ID NO: 99) |
| | | (SEQ ID NO: 98) | |
| 1112B3D7 | ACI-8016- | CAGGTCCAACTGCAGCAGTC | GATATTGTGCTAACTCAGTC |
| | 1112B3D7- | TGGGGCTGAGCTGGTGAGGC | TCCAGCCACCCTGTCTGTGA |
| | AB1 | CTGGGACTTCAGTGAAGTTG | CTCCAGGAGATAGCGTCAGT |
| | | TCCTGCAAGGCTTCTGGCTA | CTTTCCTGCAGGGCCAGCCA |
| | | CACCTTCACCAGCTACTGGA | AAGTATTAGCAACAACCTAC |
| | | TGCACTGGGTAAAGCAGAGG | ACTGGTATCAACAAAAATCA |
| | | CCTGGACAAGGCCTTGAGTG | CATGAGTCTCCAAGGCTTCT |
| | | GATCGGAGGGATTGATCCTT | CATCAAATATACTTCCCAGT |
| | | CTGATAGTTATACTAAGTAC | CCATTTCTGGGATCCCCTCC |
| | | AATCAAAAGTTCAAGGCCAA | AGGTTCAGTGGCAGTGGATC |
| | | GGCCACTTTGACTGCAGACA | AGGGACAGATTACACTCTCA |
| | | CATCCTCCAGCACAGCCTAC | GCATCAACAGTGTGGAGACT |
| | | ATGCAGCTCAGCAGCCTGAC | GAAGATTTTGGAATGTATTT |
| | | ATCTGAGGACTCTGCGATCT | CTGTCAACAGAGTAACAACT |
| | | TTTACTGTGCAAGAGAGGAG | GGCCGTACACGTTCGGAGGG |
| | | GCCTCTACGGTAGTCGCCCA | GGGACCAAGCTGGAAATAAA |
| | | CTGGTACTTCGATGTCTGGG | A |
| | | GCACAGGGACCACGGTCACC | (SEQ ID NO: 119) |
| | | GTCTCCTCA | |
| | | (SEQ ID NO: 118) | |
| 2221B7F1 | ACI-8018- | GAAGTGCAGCTGGTGGAGTC | GACATCAAGATGACCCAGTC |
| | 2221B7F1- | TGGGGGAGGCTTAGTGAAGC | TCCATCTTCCATGTATGCAT |
| | AB1 | CTGGAGGGTCCCTGAAACTC | CTCTAGGAGAGAGAGTCACT |
| | | TCCTGTGCAGCCTCTGGATT | ATCACTTGCAAGGCGAGTCA |
| | | CACTTTCAGTGACTCTTATA | GGACATTAATAGCTATTTAA |
| | | TGTATTGGCTTCGCCAGACT | GCTGGTTCCAGCAGAAACCA |
| | | CCGGAAAAGAGGCTGGAGTG | GGGAAATCTCCTAAGACCCT |
| | | GGTCGCATCCATTAGTGATG | GATCTATCGTGCAAACAGAT |
| | | GTGGTAGATACACCTATTAT | TGGTAGATGGGGTCCCATCA |
| | | TCAGACAGTGTGAAGGGGCG | AGGTTCAGTGGCAGTGGATC |
| | | ATTCACCATCTCCAGAGACA | TGGGCAAGATTATTCTCTCA |
| | | ATGTCAAGAACAACCTGTAC | CCATCAGCAGCCTGGAGTAT |
| | | CTGCAAATGACCAGTCTGAG | GAAGATATGGGAATTTATTA |
| | | GTCTGAGGACACAGCCATCT | TTGTCTACAGTATGCTGAGT |
| | | ATTACTGTGCAAGAGGTCTT | CCACGTGGACGTTCGGTGGA |
| | | GACTATTGGGGCCAAGGCAC | GGCACCAAGCTGGAAATCAA |
| | | CACTCTCACAGTCTCCTCA | A |
| | | (SEQ ID NO: 128) | (SEQ ID NO: 129) |
| 2314F6H11 | ACI-8019- | CAGGTTCAGGTGCAGCAGTC | GATGTTGTGATGACCCAGAC |
| | 2314F6H11- | TGGAGCTGACCTGGCGAGGC | TCCACTCTCCCTGCCTGTCA |
| | AB1 | CTGGGGCTTCAGTGAAACTG | GTCTTGGAGATCAAGCCTCC |
| | | TCCTGCAAGGCTTCTGGCTA | ATCTCTTGCAGATCTAGTCA |
| | | CACCTTCACAAACTATGGTA | GAACCTTATACACAGTAATG |
| | | TAAACTGGGTGAAGCAGAGA | GAATCACCTATTTACATTGG |
| | | ACTGGACAGGGCCTTGAGTG | TACCTGCAGAAGCCAGGCCA |
| | | GATTGGGGAGATTTATCCTA | GTCTCCAAAGCTCCTGATCT |
| | | AAAATATTGATATTTACTAC | ACAAAGTTTCCAACCGATTT |
| | | AATGAGAAATTCAAGGGCAA | TCTGGGGTCCCACACAGGTT |
| | | GGCCACACTGACTGCAGACA | CAGTGGCAGTGGATCAGGGA |
| | | AATCCTCCAGCACAGCGTAC | CAGATTTCACACTCAAGATC |
| | | ATGGAGCTCCGCAGCCTGAC | AGCAGAGTGGAGGCTGAGGA |
| | | ATCTGAGGACTCTGCGGTCT | TCTGGGAGTTTATTTCTGCT |
| | | ATTTCTGTGGAAGATCAAGC | CTCAAAGTACACATGTTCCG |
| | | TTTGGTTTTAGCGGGAACTA | TGGACGTTCGGTGGAGGCAC |
| | | TGCTATGGACTATTGGGGTC | CAAGCTGGAAATCAAA |
| | | AAGGAACCTCAGTCACCGTC | (SEQ ID NO: 139) |
| | | TCCTCA | |
| | | (SEQ ID NO: 138) | |
| 207E8B2 | ACI-8016- | CAGGTCCAGCTGCAGCAGTC | GATGTTTTGATGACCCAAAC |
| | 207E8B2- | TGGGGCTGAACTGGCAAAAC | TCCACTCTCCCTGCCTGTCA |
| | AB1 | CTGGGGCCTCAGTGAAGCTG | GTCTTGGAGATCAAGCCTCC |
| | | TCCTGCAAGGCTTCTGGCTA | ATCTCTTGCAGATCTAGTCA |
| | | CACCTTTACTAGCTACTGGA | GAGCATTGTCCATAGTAATG |
| | | TGCACTGGGTAAAACAGAGG | GAAACACCTATTTAGAATGG |
| | | CCTGGACAGGGTCTGGAATG | TACCTGCAGAAACCAGGCCA |
| | | GATTGGATACATTAATCCTA | GTCTCCAAAGCTCCTGATCT |
| | | GCAGTGGTTATACTAAGTGC | ACAAAGTTTCCAACCGATTT |
| | | AATCAGAAGTTCAAGGACAA | TCTGGGGTCCCAGACAGGTT |
| | | GGCCACATTGACTGCAGACA | CAGTGGCAGTGGATCAGGGA |
| | | AATCCTCCAGCACAGCCTAC | CAGTTTTCACTCTCAAGATC |
| | | ATGCAGCTGAGCAGCCTGAC | AGCAGAGTGGAGGCTGAGGA |
| | | ATATGAGGACTCTGCAGTCT | TCTGGGAGTTTATTACTGCT |
| | | ATTACTGCGTGTTCCCATTT | TTCAAGATTCACATTTTCCA |
| | | GATTACGACGGCTTTGACTA | TTCACGTTCGGCTCGGGGAC |
| | | CTGGGGCCAAGGCACCACTC | AAAGTTGGAAATAAAA |
| | | TCACAGTCTCCTCA | (SEQ ID NO: 149) |
| | | (SEQ ID NO: 148) | |
| 936.2A1B12 | ACI-8016- | GAGGTCCAGCTGCAGCAGTC | GCTATCCAGATGACCCAGAC |
| | 2A1B12- | TGGAGCTGAGCTGGTGAGGC | TACATCCTCCCTGTCTGCCT |
| | AB1 | CTGGGTCCTCAGTGAAGATG | CGCTAGGAGACAGAGTCACC |
| | | TCCTGCGAGACTTCTGGATA | ATCAGTTGCAGGGCAAGTCA |
| | | TACATTCACAACCTACGGTT | GGACATTAGCAATTATTTAC |
| | | TGACCTGGGTGAAGCAGAGG | ACTGGTATCAGCAGAAACCA |
| | | CCTGGACAGGGCCTGGAATG | GATGGAACTGTTAAACTCCT |
| | | GATTGGATATATTTATGTTG | GATCTTCTTCACATCTGGAT |
| | | GAAATGGTTATACTGAGTAT | TACGCTCAGGAGTCCCATCA |
| | | AATGAGAAGTTCAAGGACAA | AGGTTCAGTGGCAGTGGGTC |
| | | GGCCACACTGACTTCTGACA | TGGAACAGATTATTCTCTCA |
| | | CATCTTCCAGTATAGTCTAT | CCATTAGCAACCTGGAGCAA |
| | | ATGCAGCTCAGCAGCCTGAC | GAAGATATTGCCACTTATTT |
| | | ATCTGAGGACTCTGCAATCT | TTGTCAACAGAGTATTCTGC |
| | | ATTTCTGTGCAAGATTGGGA | TTCCGTGGACGTTCGGTGGA |
| | | TTACGTCGTCATTTTGACTA | GGCACCAAGCTGGAAATCAA |
| | | CTGGGGCCAAGGCACCACTC | A |
| | | TCACAGTCTCCTCA | (SEQ ID NO: 159) |
| | | (SEQ ID NO: 158) | |
| 936.17H1G2 | ACI-8016- | GAGGTCCAGCTTCAGCAGTC | GATATCCAGATGACACAGAC |
| | 17H1G2- | TGGAGCTGAGCTGGTGAGGC | TACATCCTCCCTGTCTGCCT |
| | AB1 | CTGGGTCCTCAGTGAAGATG | CTCTGGGAGACAGAGTCACC |
| | | TCCTGTAAGACTTCTGGATT | ATCAGTTGCAGTGCAAGTCA |
| | | TACATTCACAAGTTACGGTA | GGGCATTAGCAATTATTTAA |
| | | TAAGTTGGGTGATTCAGAGG | ACTGGTTTCAGCAGAAACCA |
| | | CCCGAACAGGGCCTGGAATG | AATGGAACTGTTAGACTCCT |
| | | GATTGGATATATTTATATTA | GATCTATTACACATCAAGTT |
| | | GAAATGGTTATACTGAATAT | TACACTCAGGAGTCCCATCA |
| | | AATGAGAAGTTTAAGGGCAA | AGGTTCAGTGGCAGTGGGTC |
| | | GGCCACACTGACTTCAGACA | TGGGACAGATTATTCTCTCA |
| | | CATCCTCCAACACAGCCTAC | CCATCAGCAACCTGGAACCT |
| | | ATGCAGCTCAGCAACCTGGC | GAAGATATTGCCACTTATTA |
| | | ATCTGAGGACTCTGCAATCT | TTGTCAGCAATATGATAGGC |
| | | ATTTTTGTGTAAGAATTCAT | TTCCGTGGACGTTCGGTGGA |
| | | CGACGGTTTTTTGACTACTG | GGCACCAAGCTGGAAATCAA |
| | | GGGCCAAGGCACCACTCTCA | G |
| | | CAGTCTCCTCA | (SEQ ID NO: 169) |
| | | (SEQ ID NO: 168) | |
| 936.18F4C12 | ACI-8016- | GAGGTCCAGCTTCAGCAGTC | GACATTGTGCTGACACAGTC |
| | 18F4C12- | TGGAGCTGAGCTGGTGAGGC | TCCTGCTTCCTTAGCTGTAT |
| | AB1 | CTGGGTCCTCAGTGAAGATG | CTCTGGGGCAGAGGGCCACC |
| | | ACCTGCAAGACTTCTGGATA | ATCTCATGCCGGGCCAGCCA |
| | | TTCATTCTCAAGCTACGGTA | AAGTGTCAGTACATCTGGCC |
| | | TGAGTTGGGTGAAGCAGAGG | ACAGTTATATGCACTGGTAC |
| | | CCTGGACAGGGCCTGGAATG | CAACAGAAACCCAGACAGTC |
| | | GATTGGATATATTTATGTTG | ACCCAAACTCCTCATCCATT |
| | | GAAATGGTTATACTGAATAC | TTGCATCCAACCTAGAATCT |
| | | AATGAGAAGTTCAAGGGCAA | GGGGTCCCTGCCAGGTTCAC |
| | | GGCCACACTGACTTCAGACA | TGGCAGTGGGTCTGGGACAG |
| | | CATCCTCCAGCACAGTCTAC | ACTTCACCCTCAACATCCAT |
| | | ATGGACTTTAGAAGCCTGAC | CCTGTGGAGGAGGAGGATGC |
| | | ATCTGAAGACTCTGCAATTT | TGCAACCTATTATTGTCAAC |
| | | ATTTCTGTGCAAGATTGAAT | ACAGTTGGGACCTTCCTTAC |
| | | AGGGGTAGGGCCCTTGACTT | ACGTTCGGAGGAGGGACCAA |
| | | CTGGGGCCAAGGCACCACTC | GCTGGAAATAAAA |
| | | TCACAGTCTCTTCA | (SEQ ID NO: 179) |
| | | (SEQ ID NO: 178) | |
| 936.23E5F7 | ACI-8016- | CAGGTTCAGCTGCAGCAGTC | CAAATTGTTCTCACCCAGTC |
| | 23E5F7- | TGAAGCTGAGCTGGCGAGGC | TCCAGCAATCATGTCTGCAT |
| | AB1 | CTGGGGCTTCAGTGAAACTG | CTCCAGGGGAGAAGGTCACC |
| | | TCCTGCAAGGCTTCTGGCTA | ATAACCTGCAGTGTCAGCTC |
| | | CACCTTCACAAGCTATGGTA | AAGTGTAAGTTACATGTACT |
| | | TATTCTGGGTGAAGCAGAGA | GGTTCCAGCAGAAGCCAGGC |
| | | ACTGGACAGGGCCTTGAGTG | ACTTCTCCCAAAGTCTGGAT |
| | | GATTGGAGAAATTTATCCTA | TTATAGCACATCCAACCTGG |
| | | GAAGTGGTAATACTTACTAC | CTTCTGGAGTCCCTGCTCGC |
| | | AATGAGAATTTCAGGGACAA | TTCAGTGGCAGAGGATCTGG |
| | | GGCCACACTGACTGCAGACA | GACCTCTTACTCTCTCACAA |
| | | AATCCTCCAGCACAGCGTAC | TCAGCCGAATGGAGGCTGAA |
| | | ATGGAGCTCCGCAGCCTGAC | GATGCTGCCACTTATTACTG |
| | | ATCTGAGGACTCTGCGGTCT | CCAGCAAAGGAGTAGTTACC |
| | | ATTTCTGTGCAAGACAACGG | CATTCACGTTCGGCTCGGGG |
| | | GATGACTACTGGGGCCAAGG | ACAAAGTTGGAAATAAAA |
| | | TACCACTCTCACAGTCTCCT | (SEQ ID NO: 189) |
| | | CC | |
| | | (SEQ ID NO: 188) | |
| 936.26A1G2 | ACI-8016- | CAGGTTCAGCTGCAGCAGTC | GACATCAAGATGACCCAGTC |
| | 26A1G2- | TGGAGCTGAGCTGGCGAGGC | TCCATCCTCCATGTATGCAT |
| | AB1_ | CTGGGGCTTCAGTGAAGCTG | CGCTGGGAGAGAGAGTCACT |
| | hmG2c | TCCTGCAAGGCTTCTGGCTA | ATCACTTGCAAGGCGAGTCA |
| | | CAATTTCACAACGTATGGTA | GGACATTAAAAGCTATTTAA |
| | | TAAACTGGGTGAAGCAGAGA | GCTGGTACCAGCAGAAACCA |
| | | ACTGGACAGGGCCTGGAGTG | TGGAAATCTCCTAAGACCCT |
| | | GATTGGAAAGATTTATCCTA | GATCTATTATGCAACAAGCT |
| | | GAAGTGGTAATACTCACAAC | TGGCAGATGGGGTCCCATCA |
| | | AACGAGAATTTCAAGGGCAA | AGATTCAGTGGCAGTGGATC |
| | | GGCCACACTGACTGCAGACA | TGGGCAAGATTATTCTCTAA |
| | | CATCCTCCAGCACAGCGTAC | CCATCAGCAGCCTGGAGTCT |
| | | ATGGACCTCCGCAGCCTGAC | GACGATACAGCAACTTATTA |
| | | ATCTGAGGATTCTGCGGTCT | CTGTCTACAGCATGGTGAGA |
| | | ATTTCTGTGCAAGATCCCCT | GCCCATTCACGTTCGGCTCG |
| | | TACGCTTTCTACAGTAGTAG | GGGACAAAGTTGGAAATAAA |
| | | CTACTGGTACTTCGATGTCT | A |
| | | GGGGCACAGGGACCACGGTC | (SEQ ID NO: 199) |
| | | ACCGTCTCCTCA | |
| | | (SEQ ID NO: 198) | |
| 936.32B6C7 | ACI-8016- | CAGGTTCAGCTGCAACAGTC | GACATCCAGATGAACCAGTC |
| | 32B6C7- | TGACGCTGAGTTGGTGAAAC | TCCATCCAGTCTGTCTGCAT |
| | AB1 | CTGGAGCTTCAGTGAAGATA | CCCTTGGAGACACAATTACC |
| | | TCCTGCAAGATTTCTGGCTA | ATCACTTGCCATGCCAGTCA |
| | | CACCTTCACTGACCAGACTA | GAACATTAATGTTTGGTTAA |
| | | TTCACTGGATGAATCAGAGG | GCTGGTACCAGCGGAAACCA |
| | | CCTGAACAGGGCCTGGAATG | GGAAATATTCCTAAACTATT |
| | | GATTGGATATATTTATCCTG | GATCTATAAGTCTTCCAACT |
| | | GAGATGGTAGTGCTATGTAC | TGCACACAGGCGTCCCATCA |
| | | AATGAGAAATTCAGGGGCAA | AGGTTTAGTGGCAGTGGATC |
| | | GGCCACATTGACTGCAGACA | TGGAACAGGTTTCACATTAA |
| | | AATCCTCCAGCACAGCCTAC | CCATCAGCAGCCTGCAGCCT |
| | | ATGCAGCTCAACAGCCTGAC | GAAGACATTGCCACTTACTA |
| | | ATCTGAAGACTCTGCAGTCT | CTGTCAACAGGGTCAAAGTT |
| | | ATTTCTGTGCAAGACCTCTC | ATCCTCTCACGTTCGGAGGG |
| | | TATTCCTACGCTAGTTACTA | GGGACCAAGCTGGAAATAAA |
| | | CTGGTTTGCTTACTGGGGCC | A |
| | | AAGGGACTCTGGTCACTGTC | (SEQ ID NO: 209) |
| | | TCTGCA | |
| | | (SEQ ID NO: 208) | |
| 936.22D3A6 | ACI-8016- | CAGGCTCAGCTGCAGCAGTC | CAAATTGTTCTCACCCAGTC |
| | 22D3A6- | TGGAACTGAGCTGGCGAGGC | TCCAGCAATCATGTCTGCAT |
| | AB1_ | CTGGGTCTTCAGTGAAACTG | TTCCAGGGGAGAAGGTCACC |
| | hmG2c | TCCTGCAAGGCTTCTGGCTT | ATAACCTGCAGTGCCACCTC |
| | | CACCTTCACAATTTATGGTA | AAGTGTAAGTTACATGCACT |
| | | TAAACTGGGTGAAGCAGAGA | GGTTCCAGCAGAAGCCAGGC |
| | | ACTGGACAGGGCCTTGAGTG | ACTTCTCCCAAACTCTGGAT |
| | | GATTGGAGAGATTTATCCTA | TTATAGCACATCCAACCTGG |
| | | GAAGTGGTAATACTTACTAC | CTTCTGGAGTCCCTGCTCGC |
| | | GATGAGAAGTTCAAGGGCAA | TTCAGTGGCAGTGGATCTGG |
| | | GGCCACACTGACTGCAGACA | GACCTCTCACTCTCTCACAA |
| | | AATCCACCAGCACAGCGTAC | TCAACCGAATGGAGGCTGAA |
| | | ATGGAGCTCCGCAGCCTGAC | GATGCTGCCACTTATTACTG |
| | | ATCTGAGGACTCTGCGGTCT | CCAGCAAAGGAGTCGTTACC |
| | | ATTTCTGTGCAAGATCGAGG | CATTCACGTTCGGCTCGGGG |
| | | GAATGGGAAGGGGTGTTCTG | ACAAAGTTGGAAATAAAA |
| | | GGGCCAGGGGACTCTGGTCA | (SEQ ID NO: 219) |
| | | CTGTCTCTGCA | |
| | | (SEQ ID NO: 218) | |
| 936.31F10C5 | ACI-8016- | CAGGTTCAGCTGCAGCAGTC | GACATTGTGATGACCCAGTC |
| | 31F10C5- | TGAACCTGAGCTGGCGAGGC | TCACAAATTCATGTCCACAT |
| | AB1 | CTGGGGCTTCAGTGAAACTG | CAATAGGAGACAGGGTCAGC |
| | | TCCTGCACGGCTTCTGGCTA | ATCACCTGCAAGGCCAGTCA |
| | | CACCTTCATAAACTATGGTG | GGATGTGAGTGCTGCTGTAG |
| | | TAAGCTGGGTGAGACAGAGA | CCTGGTATCAACAGAAACCA |
| | | ACTGGACAGGGCCTTGAGTG | GGTCAATCTCCTAAACTACT |
| | | GATTGGAAGAATTTATCCTA | GATTTACTCGTCATCCTTCC |
| | | GAAGTGGTAATACTCGCTTC | GGTACACTGGAGTCCCTGAT |
| | | AATGAGAAGTTCCAGGGCAA | CGCTTCACTGGCAGTGCATC |
| | | GGCCACACTGACTGCAGACA | TGGGACGGACTTCACTTTCA |
| | | CATCCTCCAGCACAGCGTAC | CCATCACCACTGTGCAGGCT |
| | | ATGGAGCTCCGCAGCCTGAC | GAAGACCTGGCAGTTTATTA |
| | | ATCTGAGGACTCTGCGGTCT | CTGTCAGCAACATTTTGATA |
| | | ATTTCTGTACAAGATTCCCT | CTCCGTACACGTTCGGAGGG |
| | | TACGCTTTCTCCGGTAATAG | GGGACCAAGCTGGAAATAAA |
| | | CTACTGGTACTTCGATGTCT | A |
| | | GGGGCACCGGGACCACGGTC | (SEQ ID NO: 229) |
| | | ACCGTCTCCTCA | |
| | | (SEQ ID NO: 228) | |
| 936.19E6D4 | ACI-8016- | GAAGTGAAGCTGGTGGAGTC | GATATTGTGCTATCTCAGTC |
| | 19E6D4- | TGGGGGAGGCTTAGTGCAGC | TCCAGCCACCCTATCTGTGA |
| | AB1 | CTGGAGGGTCCCTGAAACTC | CTCCAGGAGATAGCGTCAGT |
| | | TCCTGTGCAGCCTCTGGATT | CTTTCCTGCAGGGCCAGTCA |
| | | CACTTTCAGTGACTATTACA | GAGTATTGGCGACAACTTAC |
| | | TGTATTGGGTTCGCCAGAAT | ACTGGTATCAACAAAAGTCA |
| | | GCAGAGAAGAGGCTGGAGTG | CATGAGTCTCCAAGGCTTAT |
| | | GGTCGCATACATCAGTAGTG | CATCAAGTCTGCTTCCCAGT |
| | | GCGGTGATGCCCCCTTTTAT | CCATCTCTGGGATCCCCTCC |
| | | TCAGACAATTTGAAGGGCCG | AGGTTCAGTGGCAGTGGATC |
| | | ATTCACCATCTCCAGAGACA | AGGGACAGATTTCACTCTCA |
| | | ATGCCAAGAACACCCTGTAC | GTATCAACATTGTGGAGACT |
| | | CTGCAAATGAGCCGTCTGCA | GAAGATTTTGGAATGTATTT |
| | | GCCTGAGGACACAGCCATGT | CTGTCAACAGAGTGACACCT |
| | | ATTTCTGTGCAAGAGAGGGG | GGCCTCTCACGTTCGGTCCT |
| | | TITTATTACTACGGTCTTTA | GGGACCAAGCTGGAGCTGAA |
| | | TGGTATGGATTATTGGGGTC | A |
| | | AAGGAACCTCAGTCACCGTC | (SEQ ID NO: 239) |
| | | TCCTCA | |
| | | (SEQ ID NO: 238) | |
| 936.3E6B11 | ACI-8016- | GAGGTCCAGCTTCAGCAGTC | GACATTGTACTGACACAGTC |
| | 3E6B11- | TGGAGCTGAACTGGTGAGGC | TCCTGCTTCCTCAGCTGTCT |
| | AB1 | CTGGGTCCTCAGTGAAGATG | CTCTGGGACAGAGGGCCACC |
| | | TCCTGCAAGACTTCTGGATA | ATCTCATGCAGGGCCAGCCA |
| | | TACATTCACAAATTACGGTA | AAGTGTCAGTACATCTAGTT |
| | | TAACCTGGGTGAAACAGAGG | ATAGTTATATGCACTGGTAC |
| | | CCTGGACAGGGCCTGGAATG | CAACAGAAACCCGGACAGCC |
| | | GATTGGATATATTTATGTTG | ACCCAAACTCCTCATTAAAT |
| | | GAAACGGTTATACTGAGTAT | ATGCATCCACCCTTGAATCT |
| | | AATGAGAAGTTCAAGGACAA | GGGGTCCCTGCCAGGTTCAG |
| | | GGCCGCGCTGACTTCAGACA | TGGCAGTGGCTCTGGGACAG |
| | | CATCCTCCAGCACAGCCTAC | ACTTCACCCTCAACATCCAT |
| | | ATGCAGCTCACCAGCCTGAC | CCTGTGGAGGAGGAGGATAC |
| | | ATCTGACGACTCTACAATCT | TGCAACATATTACTGTCAGC |
| | | ATTTCTGTGCCAGAATAGCT | ACAGTTGGGAACTTCCGTAC |
| | | CAGGGAAGATGCTTTGACTA | ATGTTCGGAGGGGGGACCAA |
| | | CTGGGGCCAAGGCACCACTC | GCTGGAAATAAAA |
| | | TCTCAGTCTCCTCA | (SEQ ID NO: 249) |
| | | (SEQ ID NO: 248) | |
| 936.11A3F3 | ACI-8016- | CAGGTTCAGCTGCAGCAATC | GACATCCAGATGACTCAGTC |
| | 11A3F3- | TGGAGCTGAGCTGGCGCGGC | TCCAGCCTCCCTGGCTGCAT |
| | AB1 | CTGGGACTTCAGTGAAGCTG | CTGTGGGAGAAACTGTCACC |
| | | TCCTGCAAGGCTTCTGGCTA | ATCACATGTCGAGCAAGTGA |
| | | CACTTTCATAAGCTTTGGTT | GAACATTTACTACAGTTTAG |
| | | TAAGCTGGGTGAAACAGAGA | CATGGTATCAGCAGAAGCAA |
| | | ACTGGACAGGGCCTTGAGTG | GGGAAATCTCCTCAGGTCCT |
| | | GATTGGAGAGATTTATCCTA | GATCTATTATGCAAACAGCT |
| | | GAATTGATAATCCTTACTAC | TGGAAGATGGTGTCCCATCG |
| | | AATGAGAAGTTCAAGGGCAA | AGGTTCAGTGGCAGTGGATC |
| | | GGCCATACTGACTGCAGACA | TGGGACACAGTATTCTTTGA |
| | | AATCCTCCAGCACAGCGTAC | AGATCAATAGCATGCAGCCT |
| | | ATGGAGCTCCGCAGCCTGAC | GAAGATACCGCAACTTATTT |
| | | ATCTGAGGACTCTGCGGTCT | CTGTAAACAGGCTTATGACG |
| | | ATTTCTGTGCAAGATACTTT | TTCCGTTCACGTTCGGTACT |
| | | ACTAAGGGAAACTACTTTGA | GGGACCAAGCTGGAGCTGAA |
| | | CTACTGGGGCCAAGGCACCA | A |
| | | CTCTCACAGTCTCCTCA | (SEQ ID NO: 259) |
| | | (SEQ ID NO: 258) | |
| 936.14G5B8 | ACI-8016- | GAAGTCCAGTTGCAAGAATC | GACATTGTGATGACCCAGTC |
| | 14G5B8- | TGGACCTGAACTGGTGAAGC | TCAAAAATTCATGTCCACAT |
| | AB1 | CTGGGTCTTCAGTGAAGATA | CAGTAGGGGACAGGGTCAGC |
| | | TCCTGTAAGGCTTCTGGATA | CTCACCTGCAAGGCCAGTCA |
| | | CACGTTCGCTGACCACTACA | GATTGTGGGTACTGCTGTAG |
| | | TGAATTGGGTGAAGCAGAGC | CCTGGTATCAACAGAAACCA |
| | | CATGGAAAGAGCCTTGAGTG | GGACAATCTCCTAAACTACT |
| | | GATTGGAGATATTAATCCTA | GATTTACTCGGCGTCCATTC |
| | | ACAATGGTGGTTCTGTCTAC | GGTACACTGGAGTCCCTGAT |
| | | AACCAGAAGTTCAAGGGCAA | CGCTTCACAGGCAGTGGATC |
| | | GGCCACATTGACTGTAGACA | TGGGACAGATTTCACTCTCA |
| | | AGTCCTCCAGCACAGCCTAC | CCATCAGCAATATGCAGTCT |
| | | ATGGAACTCCGCAGCCTGAC | GAAGACCTGGCAGATTATTT |
| | | ATCTGAGGACTCTGCAGTCT | CTGCCAGCAATATAGCAACT |
| | | ATTTCTGTGCAAGAAGGAGG | ATCCCACGTTCGGTGCTGGG |
| | | GCACATTATTACGACGGTCC | ACCAAGCTGGAACTGAAA |
| | | TTACTTTGACTACTGGGGCC | (SEQ ID NO: 269) |
| | | AAGGCACCACTCTCACAGTC | |
| | | TCCTCA | |
| | | (SEQ ID NO: 268) | |
| 936.27A1G4 | ACI-8016- | CAGGTTCAGCTGCAACAGTC | GACATCCAGATGAACCAGTC |
| | 27A1G4- | TGACGCTGAGTTGGTGAAAC | TCCATCCAGTCTGTCTGCAT |
| | AB1 | CTGGAGCTTCAGTGAAGATA | CCCTTGGAGACACAATTACC |
| | | TCCTGCAAGGTTTCTGGCTA | ATCACTTGCCATGCCAGTCA |
| | | CACCTTCACTGACCATACTA | GAACATTAATGTTTGGTTGA |
| | | TTCACTGGATGAGGCAGAGG | CCTGGTACCAGCAGAAACCA |
| | | CCTGAACAGGGCCTGGAATG | GGAAATATTCCTAAACTTTT |
| | | GATTGGATATATTTATCCTG | GATCTATAAGGCTTCCAACT |
| | | GAGATGCTAGTAGTTACTAC | TGCACACAGACGTCCCATCA |
| | | AATGAGAAGTTCAAGGGCAA | AGGTTTAGTGGCAGTGGATC |
| | | GGCCACATTGACTGCAGACC | TGGAACAGGTTTCACATTAA |
| | | AATCCTCCAACACAGCCTAC | CCATCAGCAGCCTGCAGCCT |
| | | ATACAGGTCAACAGCCTGAC | GAAGACATTGCCACTTACTA |
| | | ATCTGAGGACTCTGCAGTCT | CTGTCAACAGGGTCAAAGTT |
| | | ATTTCTGTGCAAGACCTCTC | ATCCTCTCACGTTCGGAGGG |
| | | TATTCCTACGCTAGTTACTA | GGGACCAAGCTGGAAATAAA |
| | | CTGGTTTGCTTACTGGGGCC | A |
| | | AAGGGACTCTGGTCACTGTC | (SEQ ID NO: 279) |
| | | TCTGCA | |
| | | (SEQ ID NO: 278) | |
| 936.29C5E11 | ACI-8016- | GAGGTCCAGCTTCAGCAGTC | GACATTGTGCTGACACAGTC |
| | 29C5E11- | TGGAGCTGAGCTGGTGAGGC | TCCTGCTTCCTTAGCTGTAT |
| | AB1 | CTGGGTCCTCAGTGAAGATG | CTCTGGGGCAGAGGGCCACC |
| | | TCCTGCAGGACTTCTGGATA | ATCTCATGCAGGGCCAGCAA |
| | | CACATTCACAAACAACGGTA | AAGTGTCAGTTCATCTGCCT |
| | | TAACCTGGGTGAAGCAGAGG | ATAGTTATATGCACTGGTAC |
| | | CCTGGACAGGGCCTGGAATG | CAACAGAAACCAGGACAGCC |
| | | GATTGGATATATTTATGTTG | ACCCAAACTCCTCATCCATC |
| | | GAAATGGTTATACTGAATAC | TTGCATCCAACCTAGAATCT |
| | | AATGAGAAGTTCAAGGACAA | GGGGTCCCTGCCAGGTTCAG |
| | | GGCCACTCTGACTTCAGACA | TGGCAGTGGGTCTGGGACAG |
| | | CAGCCTCCAGTACAGTTTAC | ACTTCACCCTCAATATCCAT |
| | | ATGCAGCTCAACAGCCTGAC | CCTGTGGAGGAGGAAGATGC |
| | | ATCTGAGGACTCTGCAATCT | TGCAACCTATTACTGTCAGC |
| | | ATTTTTGTGCAAGATTGGAT | ACAGTTGGGAGCTTCCTTAC |
| | | AGGGGTAGGGCCCTTGACTA | ACGTTCGGAGGGGGGACCAA |
| | | CTGGGCCCAAGGCACCACTC | GCTGGAAATAAAA |
| | | TCACAGTCTCCTCA | (SEQ ID NO: 289) |
| | | (SEQ ID NO: 288) | |
| 936.7G3B5 | ACI-8016- | GAGGTTCAGCTGCAGCAGTC | GATGTTGTACTAACCCAGTC |
| | 7G3B5- | TGTGGCAGAGCTTGTGAGGC | TCCAGCCACCCTGTCTGTGA |
| | AB1 | CAGGGGCCTCAGTCAAGTTG | CTCCAGGAGATAGCGTCAGT |
| | | TCCTGCACAGCTTCTGGCTT | CTTTCCTGCAGGGCCAGCCA |
| | | CAACATTAAAAACACCTATA | AAGTATTTACAACAACCTAC |
| | | TGCACTGGGTGAAACAGAGG | ACTGGTATCAACAAAAATCA |
| | | CCTGAACAGGGCCTGGAGTG | CATGAGTCTCCAAGGCTTCT |
| | | GATTGGAAGGATTGATCCTG | CGTCAAGTATGCTTCCCAGT |
| | | CGAATGGTAATACTAAATAT | CCATCTCTGGGATCCCCTCC |
| | | GTCCCGAAGTTCCAGGGCAA | AGGTTCAGTGGCAGTGGATC |
| | | GGCCACTATAACTGCAGACA | GGGGACAGATTTCACTCTCA |
| | | CATCTTCTAAGACAGCCTAC | GTATCAACAGTGTGGAGACA |
| | | CTGCAGCTCAGCAGCCTGAC | GAAGATTTTGGAATGTATTT |
| | | ATCTGAGGACACTGCCATCT | CTGTCAACAGAGTAACAGCT |
| | | ATTACTGTACTAGATGGGGT | GGCCTCTCACGTTCGGTGCT |
| | | TTTGATAAAGACGGGGTGGT | GGGACCAAGCTGGAGCTGAA |
| | | CTATGCCATGGACTATTGGG | A |
| | | GTCAAGGAACCTCAGTCACC | (SEQ ID NO: 299) |
| | | GTCTCCTCA | |
| | | (SEQ ID NO: 298) | |
| 2504F3D9 | ACI-8016- | GACGTGAAGCTGGTGGAATC | GACATCAAGATGACCCAGTC |
| | 2504F3D9- | TGGGGAAGGCTTAGTGAAGC | TCCATCTTCCATGTATGCAT |
| | AB1 | CTGGAGGGTCCCTGAAACTC | CTCTAGGAGAGAGAGTCACT |
| | | TCCTGTGCAGTCTCTGGATT | ATCACTTGCAAGGCGAGTCA |
| | | CACTTTCAGTAACTATGCCA | GGACATTAAAAACTATTTAA |
| | | TGTCTTGGGTTCGCCAGACT | GCTGGTTCCAACAGATACCA |
| | | CCAGAGAAGAGGCTGGAGTG | GGGAAATCTCCTAAGACCCT |
| | | GGTCGCATATATTAGTAGTG | GATCTATCGTGCAAACAGAT |
| | | GTGGTGATTTCATCTACTAT | TGGTAGATGGGGTCCCATCA |
| | | AGAGACACTGTGAAGGGCCG | AGGTTCAGTGGCAGTGGATC |
| | | ATTCACCATCTCCAGAGACA | TGGGCAAGATTATTCTCTCA |
| | | ATGCCAGGAACACCCTGTAC | CCATCAGCAGCCTGGAGTAT |
| | | CTGCAAATGAGCAGTCTGAA | GAAGATATGGGAATTTATTA |
| | | GTCTGAGGACACAGCCATGT | TTGTCTACAGTATTATGAGT |
| | | ATTATTGTATTTTACAACTA | TTCCGTACACGTTCGGAGGG |
| | | CGGCCAGAAGCCTGGTTTGC | GGGACCAAACTGGAAATAAA |
| | | TTACTGGGGCCAAGGGACTC | A |
| | | TGGTCACTGTCTCTGCA | (SEQ ID NO: 309) |
| | | (SEQ ID NO: 308) | |
| 2516A8C6 | ACI-8016- | CAGGTTCAGCTGCAGCAGTC | CAAATTGTTCTCACCCAGTC |
| | 2516A8C6- | TGGAGCTGAGCTGGCGAGGC | TCCAGTAATCATGTCTGCAT |
| | AB1 | CTGGGGCTTCAGTGAAGCTG | CTCCAGGGGAGAAGGTCACC |
| | | TCCTGCAAGGCTTCTGGCCA | ATAACCTGCAGTGCCAGCTC |
| | | CACCTTCACAAGCTATGGTA | AAGTGTAAGTTACATGTACT |
| | | TAAGTTGGGTGAAGCAGAGA | GGTTCCAGCAGAAGCCAGGC |
| | | ACTGGACAGGGCCTTGAGTG | ACTTCTCCCAAATTCTGGAT |
| | | GATTGGAAAGATTTATCCTA | TTATAGCACATCCAACCTGG |
| | | GAAGCGGTAATACTTACTAC | CTTCTGGAGTCCCTGCTCGC |
| | | GATGAGAAGTTCAAGGGCAA | TTCAGTGGCAGTGGATCTGG |
| | | GGCCACACTGACTGCAGACA | GACCTCTTACTCTCTCACAA |
| | | AACCCTCCACCACAGCGTAC | TCAGCCGAATGGAGGCTGAA |
| | | ATGGAGCTCCGCAGCCTGAC | GATGCTGCCACTTATTACTG |
| | | ATCTGAGGACTCTGCGGTCT | CCAGCAAAGGAGTAGGTACC |
| | | ATTTCTGTGCAACCAGGGAC | CGTGGACGTTCGGTGGAGGC |
| | | TACTGGGGCCAAGGCACCAC | ACCAAGCTGGAAATCAAG |
| | | TCTCACAGTCTCCTCA | (SEQ ID NO: 319) |
| | | (SEQ ID NO: 318) | |
| 2602H6F10 | ACI-8016- | CAGGTTCAGCTGCAGCAGTC | GACATCCAGATGAACCAGTC |
| | 2602H6F10- | TGGAGCTGAACTGGCGAGGC | TCCATCCAGTCTGTCTGCAT |
| | AB1 | CTGGGGCTTCAGTGAAGCTG | CCCTTGGAGACACAATCACC |
| | | TCCTGCAAGGCTTCTGGCTT | ATCAATTGTCATGCCAGTCA |
| | | CAGTTTCACAAGTTATGGTA | GAACATTAATGTTTGGTTAA |
| | | TAAGTTGGGTGAAGCAGAGA | GCTGGTACCAGCAGAAACCA |
| | | ACTGGACAGGGCCTTGAGTG | GGAAATATTCCTAAAGTTTT |
| | | GATTGGACAGATTTATCTTA | GATCTATCAGGCTTCCAACT |
| | | GAAGTAGTAATACTTACTAC | TGCAGACAGGCGTCCCAACA |
| | | AATGAGAAGTTCAAGGACAA | AGATTTAGTGGCAGTGGATC |
| | | GGCCACACTGACTGCAGACA | TGGAACAGGTTTCACTTTAA |
| | | AATCCTCCAGCACAGTGTAC | CCATCAGCAGACTGCAGCCT |
| | | ATGGAGCTCCGCAGCCTGAC | GAAGACATTGCCACTTACTA |
| | | ATCTGAGGACTCTGCGGTCT | CTGTCAGCAGGGTCAAAGAT |
| | | ATTTCTGTGCAAGATACTAC | ATCCGTGGACGTTCGGTGGA |
| | | GGTAGTAGTTCGGACTACTG | GGCACCAAGCTGGAAATCAA |
| | | GGGCCAAGGCACCACTCTCA | G |
| | | CAGTGTCCTCA | (SEQ ID NO: 329) |
| | | (SEQ ID NO: 328) | |
| 2609F4A9 | ACI-8016- | CAGGTTCAGCTGCAACAGTC | GACATCCAGATGAACCAGTC |
| | 2609F4A9- | TGACGCTGAGTTGGTGAAAC | TCCATCCAGTCTGTCTGCCT |
| | AB1 | CTGGAGCTTCAGTGAAGATA | CCCTTGGAGACACAATTACC |
| | | TCCTGCAAGGTTTCTGGCTA | ATCACTTGCCATGCCAGTCA |
| | | CGCCCTCACTGACCATTCTA | GAACATTAATGTTTGGTTAA |
| | | TTCATTGGATGAAGCAGAGG | ACTGGTACCAGCAGAAACCA |
| | | CCTGAACAGGGCCTGGCATG | GGAAATATTCCTAAACTATT |
| | | GATTGGATATATGTATCCTG | GATCTATAAGGCTTCCAAGT |
| | | GAGATGGTAGTGCTAACTAC | TGCACACAGGCGTCTCATCA |
| | | AATGACAAATTCAGGGGCAA | AGCTTTAGTGGCAGTGGATC |
| | | GGCCACATTGACTGCAGACA | TGGAACAGGTTTCACATTAA |
| | | TATCCTCCAGCACAGCCTAC | CCATCAGCAGCCTACAGCCT |
| | | ATGCACCTCAACAGCCTGAC | GAAGACATTGGCACTTACTA |
| | | ATCTGAGGACTCTGCAGTCT | CTGTCAACAGGGTCAAAGTT |
| | | ATTTCTGTACAAGACCCTAT | ATCCGTACACGTTCGGAGGG |
| | | TCCTTCGGCAGTAGTTACGA | GGGACCAAGCTGGAGATAAA |
| | | CTACTTTGGCTACTGGGGCC | A |
| | | AAGGCACCACTCTCACAGTC | (SEQ ID NO: 339) |
| | | TCCTCA | |
| | | (SEQ ID NO: 338) | |
| 2610H7D3 | ACI-8016- | CAGGTTCAGCTGCAGCAGTC | GACATCCAGATGAACCAGTC |
| | 2610H7D3- | GGGAGTTGAACTGGCGAGGC | TCCATCCAGTCTGTCTGCAT |
| | AB1 | CTGGGGCTTCAGTGAAGTTG | CCCTTGGAGACACAATCACC |
| | | TCCTGCAAGGCTTCGGGCTT | ATCAATTGCCATGCCAGTCA |
| | | CAGTTTTACAAGTTATGGTA | GAACATTAATGTTTGGCTAA |
| | | TAAGCTGGGTGAAGCAGAAA | GCTGGTACCAGCAGAAACCA |
| | | ACTGGACAGGGCCTTGAGTG | GGAAATATTCCTAAAGTTTT |
| | | GATTGGACAGATTTATCTTA | GATCAATCAGGCTTCCAACT |
| | | GAAGTAGTAATACTTATTAC | TGCAGACAGACGTCCCAACA |
| | | AGTGAGAAGTTCAAGGACAG | AGATTTAGTGGCAGTGGATC |
| | | GGCCACACTGACTGCAGACA | TGGAACAGATTTCAAATTAA |
| | | AATCCTCCAGCACAGTGTAC | TCATCAGCAGACTGCAGTCT |
| | | ATGGAGCTCCGCAGCCTGAC | GAGGACATTGCCACTTACTA |
| | | ATCTGAGGACTCTGCGGTCT | CTGTCAGCAGGGTCAAAGTT |
| | | ATTTCTGTGCAAGATATTAC | ATCCGTGGACGTTCGGTGGA |
| | | GGTAGTAGTTCGGACTACTG | GGCACCAAGCTGGAAATCAA |
| | | GGGCCAAGGCACCACTCTCA | A |
| | | CAGTCTCCTCA | (SEQ ID NO: 349) |
| | | (SEQ ID NO: 348) | |
| 2614C3B2 | ACI-8016- | CAGGTTCAGCTGCAACAGTC | GACATCCAGATGAACCAGTC |
| | 2614C3B2- | TGACGCTGAGTTGGTGAAAC | TCCATCCAGTCTGTCTGCCT |
| | AB1 | CTGGAGCTTCAGTGAAGATA | CCCTTGGAGACACAATTACC |
| | | TCCTGCAAGGTTTCTGGCTA | ATCACTTGCCATGCCAGTCA |
| | | CGCCCTCACTGACCATTCTA | GAACATTAATGTTTGGTTAA |
| | | TTCACTGGATGAAGCAGAGG | GCTGGTACCAGCAGAAACCA |
| | | CCTGAACAGGGCCTGGCATG | GGAAATGTTCCTAAATTATT |
| | | GATTGGATATATGTATCCTG | GATCTATAAGGCTTCCAAGT |
| | | GAGATGGTAGTGCTAATTAC | TGCACACAGGCGTCTCATCA |
| | | AATGACAAATTCAAGGGCAA | AACTTTAGTGGCAGTGGATC |
| | | GGCCACATTGACTGCAGACA | TGGAACAGGTTTCACATTAA |
| | | TATCCTCCAGCACAGCCTAC | CCATCAGCAGCCTGCAGCCT |
| | | ATGCAGCTCAACAGCCTGAC | GAAGACATTGGCACTTACTA |
| | | ATCTGAGGATTCTGCAGTCT | CTGTCAACAGGGTCAAAGTT |
| | | ATTTCTGTACAAGACCCTAT | ATCCGTACACGTTCGGAGGG |
| | | TACTTCGGCAGTAGTTACGA | GGGACCAAGCTGGAGATAAA |
| | | CTACTTTGGCTACTGGGGCC | A |
| | | AAGGCACCACTCTCACAGTC | (SEQ ID NO: 359) |
| | | TCCTCA | |
| | | (SEQ ID NO: 358) | |
| 2617C3A8 | ACI-8016- | CAGGTTCAGCTGCAGCAGTC | CAAATTGTTCTCACCCAGTC |
| | 2617C3A8- | TGGAGTTGAGCTGGCGAGGC | TCCAGCAATCATGTCTGCAT |
| | AB1 | CTGGGGCTTCAGTGAAGCTG | CTCCAGGGGAGAAGGTCACC |
| | | TCCTGCAAGGCTTCTGGCTC | ATAACCTGCAGTGCCAGCTC |
| | | CACCTTCACAAGCTATAGTA | AAGTGTGAGTTACATGTACT |
| | | TAACCTGGGTGAAGCAGAGA | GGTTCCAGCAGAAGCCAGGC |
| | | ACTGGACAGGGCCTTGAGTG | ACTTCTCCCAAATTCTGGAT |
| | | GATCGGAGTGATTTATCCTA | TTATAGCACATCCAACCTGG |
| | | GAAGTGATAATACTTACTAC | CTTCTGGAGTCCCTGCTCGC |
| | | GATGAGAAGTTCAAGGGCAA | TTCAGTGGCAGTGGATCTGG |
| | | GGCCACACTGACTGCAGACA | GACCTCTTACTCTCTCACAA |
| | | AATCCTCCAGCACAGCGTAC | TCAGCCGAATGGAGGCTGAA |
| | | ATAGAGCTCCGCAGCCTGAC | GATGCTGCCACTTATTACTG |
| | | ATCTGAGGACTCTGCGGTCT | CCAGCAAAGGAGTAATTACC |
| | | ATTTCTGTGCGACACGGGAC | CATTCACGTTCGGCTCGGGG |
| | | TACTGGGGCCAAGGCACCAC | ACAAAGTTGGAAATAAAA |
| | | TCTCACAGTCTCCTCA | (SEQ ID NO: 369) |
| | | (SEQ ID NO: 368) | |
| 2622E12F11 | ACI-8016- | CGGGTCCAACTGCAGCAGGC | GACATTGTGCTGACACAGTC |
| | 2622E12F11- | TGGGGCTGAACTGGTGAAGC | TCCTGCTTCCTTAGCTGTAT |
| | AB1 | CTGGGGCTTCAGTGAAGGTT | CTCTGGGGCAGAGGGCCACC |
| | | TCCTGCAAGGCCTCAGGCAA | ATCTCATGCAGGGCCAGCAA |
| | | CACCTTCAACAACTACTGGA | AAGTGTCAGTTCTTTTGGCT |
| | | TATATTGGGTGAAACAGAGG | ATAATTATATACACTGGTAC |
| | | CCTGGCCAAGGCCTTGAGTG | CTACAGAAACCAGGACAGCC |
| | | GATTGGAAGGATTCATCCTT | ACCCAAACTCCTCATCTATC |
| | | CTGATAGTGGAACTAACTAC | TTGCATCCGACCTAGAATCT |
| | | AATCAAAAGTTCGAGGGCAA | GGGGTCCCTGCCAGGTTCAG |
| | | GGCCACATTGACTGTTGACA | TGGCAGTGGGTCTGGGACAG |
| | | AATCCTCCGGCACTGCCTAC | GCTTCACCCTCAACATCCAT |
| | | ATGCAGCTCAGCAGCCTGAC | CCTGTGGAGGAAGAGGATGC |
| | | AGCTGAGGACTCTGCGGTCT | TGCAACCTATTACTGTCAGC |
| | | ATTACTGTGCACTCCTATCG | ACAGTAGGGAGTTTCCGTGG |
| | | CTCTATGCTATGGACTCCTG | ACGTTCGGTGGAGGCACCAA |
| | | GGGTCACGGAACCTCAGTCA | GCTGGAAATCAAA |
| | | CCGTCTCCTCA | (SEQ ID NO: 379) |
| | | (SEQ ID NO: 378) | |
| 2626B9D3 | ACI-8016- | CAGGTCCAACTGCAGCAGCC | AACATTGTAATGACCCAATA |
| | 2626B9D3- | TGGGACTGAATTGGTGAAGC | TCCCAAATCCATGTCCCTGT |
| | AB1 | CTGGGGCCTCAGTGAAACTG | CAATAGGAGAGAGGGTCACC |
| | | TCCTGCAGGGCTTCTGGCTG | TTGAGGTGCAAGGCCAGTGA |
| | | GACGCACTGGGTGAAACAGA | AAATGTGGGTAGTTATGTTT |
| | | GGCCTGGACAAGGCCTTGAG | CGTGGTATCAACAGAAACCA |
| | | TGGATTGGAAATATTAATCT | GAGCAGTCTCCAAAATTGCT |
| | | TAGCAATGGTCGTACCAACT | GATATACGGGGCTTCCAACC |
| | | ACAATGAGAAGTTCAGGGCC | GTTACACTGGGGTCCCCGAT |
| | | ATGGCCACGCTGACTGTAGA | CGCTTCACTGGCAGTGGATC |
| | | CAAATCCTCCAGCACAGCCT | TGCAACAGATTTCATTCTGA |
| | | ACACGCAGCTCAGCAGCCTG | CCATCACCAGTGTGCAGGCT |
| | | ACTTCTGAGGACTCTGCGGT | GAAGACCTTGCAGATTATCA |
| | | CTATTATTGTGCAAACTCGA | CTGTGGACAGAGTTACAACT |
| | | TGGTGCCGCATTTTTTTCCT | ATCCGCTCACGTTCGGTGCC |
| | | ATGGACTATTGGGGTCAAGG | GGGACCAAGCTGGAGCTGAA |
| | | AACCTCAGTCACCGTCTCCT | A |
| | | CA | (SEQ ID NO: 389) |
| | | (SEQ ID NO: 388) | |
| 2629E8D1 | ACI-8016- | CAGGTTCAGCTGCAGCAGTC | GACATCCAGATGAACCAGTC |
| | 2629E8D1- | TGACGCTGAGTTGGTGAAAC | TCCATCCAGTCTGTCTGCAT |
| | AB1 | CTGGAGGTTCAGTGAAGATA | CCCTTGGAGACACAATTACC |
| | | TCCTGCAAGGTTTCTGGCTA | ATCACTTGCCATGCCAGTCA |
| | | CACCTTCACTGACCATACTA | GAACATTAATGTTTGGTTAA |
| | | TTCACTGGATGAAGCAGAGG | ACTGGTACCAGCAGAAACCA |
| | | CCTGAACAGGGCCTGGAATG | GGAAATATTCCTAAACTATT |
| | | GATTGGAAATATTTATCCTG | GATCTATAAGGCTTCCAACT |
| | | GAGATGGTAGTACTATGTAC | TGCACACAGGCGTCCCATCA |
| | | AATGAGAAGTTCAAGGGCAA | AGATTTAGTGGCAGTGGCTC |
| | | GGCCACATTGACTGCAGACA | TGGAATAGGTTTCACATTAA |
| | | AATCCTCCAGCACAGCCTAC | CCATCAGCAGCCTGCAGCCT |
| | | ATGCACCTCAACAGCCTGAC | GAAGACATTGCCACTTACTA |
| | | ATCTGAGGACTCTGCAGTCT | CTGTCAACAGGGTCAAAGTT |
| | | ATTTCTGTGCAAGACCCTAT | ATCCTCTGACGTTCGGTGGA |
| | | TACTTCGGTAGTGCCTCGTA | GGCACCAAGCTGGAAATCAA |
| | | CTACTTTGGCTACTGGGGCC | A |
| | | AAGGCACCACTCTCACAGTC | (SEQ ID NO: 399) |
| | | TCCTCA | |
| | | (SEQ ID NO: 398) | |

TABLE 17
Amino acid sequences of the Heavy Chain and Light Chain Variable Domains (VH and VL)
and their CDRs

| | Anti- | | | | | | | | |
|---|---|---|---|---|---|---|---|---|---|
| | body | VH | | | | VL | | | |
| Hybridoma | Name | sequence | CDR1 | CDR2 | CDR3 | sequence | CDR1 | CDR2 | CDR3 |
| 416E6G4 | ACI- | QVQLKQSGPGLV | SYGLH | VIWS | NEV | DIVLTQSPASLA | RASE | ATSN | QQSK |
| | 8016- | QPSQSLSITCTV | (SEQ | GGNT | | VSLGQRATISCR | SVDN | QGS | EVPW |
| | 416E6 | SGISLSSYGLHW | ID | DYSA | | ASESVDNYGISF | YGIS | (SEQ | T |
| | G4- | VRQSPGKGLEWL | NO: | TFIS | | INWFQQKPGQPP | FIN | ID | (SEQ |
| | AB1 | GVIWSGGNTDYS | 11) | (SEQ | | KLIIYATSNQGS | (SEQ | NO: | ID |
| | | ATFISRLSISKD | | ID | | GVPARFSGSGSG | ID | 16) | NO: |
| | | NSKSQVFFKMDS | | NO: | | TDFSLNIHPMEE | NO: | | 17) |
| | | LQVDDTAIYYCA | | 12) | | DDTAMYFCQQSK | 15) | | |
| | | RNEVWGQGTSVT | | | | EVPWTFGGGTKL | | | |
| | | VSS | | | | EIK | | | |
| | | (SEQ | | | | (SEQ | | | |
| | | ID | | | | ID | | | |
| | | NO: | | | | NO: | | | |
| | | 10) | | | | 14) | | | |
| 402H11C9 | ACI- | QVQLQQSGAELA | TYTMH | YINP | GGNP | DIVLTQSPATLS | RASQ | YASQ | QQSK |
| | 8016- | RPGASVKMSCKA | (SEQ | SSGY | AWFA | VTPGDSVSLSCR | SIGN | SIS | SWPW |
| | 402H1 | SGYTFTTYTMHW | ID | TYYN | Y | ASQSIGNNLHWY | NLH | (SEQ | T |
| | 1C9- | VKQRPGQGLEWI | NO: | QNFK | (SEQ | QHKSHESPRLLI | (SEQ | ID | (SEQ |
| | Ab1 | GYINPSSGYTYY | 21) | D | ID | KYASQSISGIPS | ID | NO: | ID |
| | | NQNFKDKATLTA | | (SEQ | NO: | RFSGSESGTDFT | NO: | 26) | NO: |
| | | DKSSSTAYMQLS | | ID | 23) | LSINSVETEDFG | 25) | | 27) |
| | | NLASDDSAVYYC | | NO: | | IYFCQQSKSWPW | | | |
| | | ALGGNPAWFAYW | | 22) | | TFGGGTKLEIK | | | |
| | | GQGTLVTVSA | | | | (SEQ | | | |
| | | (SEQ | | | | ID | | | |
| | | ID | | | | NO: | | | |
| | | NO: | | | | 24) | | | |
| | | 20) | | | | | | | |
| 203B12C3 | ACI- | QVQLKQSGPGLV | HYGVH | VIWS | KGDF | DIQMTQTSSSLS | RASQ | YTSK | QQGN |
| | 8016- | QPSQSLSITCKV | (SEQ | GGTT | YGDY | ASLGDRVTISCR | DISN | VHS | TRPF |
| | 203B1 | SDFSLSHYGVHW | ID | EYNE | FDY | ASQDISNYLNWY | YLN | (SEQ | T |
| | 2C3- | VRQSPGKGLEWL | NO: | AFIS | (SEQ | QQRPDGTVKLLI | (SEQ | ID | (SEQ |
| | AB1 | GVIWSGGTTEYN | 31) | (SEQ | ID | YYTSKVHSGVPS | ID | NO: | ID |
| | | EAFISRLTISKD | | ID | NO: | RFSGSGSGTDFS | NO: | 36) | NO: |
| | | NSKSQILFRMNS | | NO: | 33) | LTISNLEQEDFA | 35) | | 37) |
| | | LQPADTAIYYCA | | 32) | | TYYCQQGNTRPF | | | |
| | | RKGDFYGDYFDY | | | | TFGGGTKLEIK | | | |
| | | WGQGTTLTVSS | | | | (SEQ | | | |
| | | (SEQ | | | | ID | | | |
| | | ID | | | | NO: | | | |
| | | NO: | | | | 34) | | | |
| | | 30) | | | | | | | |
| 421B10C12D2 | ACI- | EVQLQQSGADLV | DTYIH | RIDP | HDGY | DIVMSQSPSSLA | QSSQ | WAST | KQSY |
| | 8016- | KPGASVKLSCTA | (SEQ | ANGN | DMDY | VSAGEKVTMSCQ | SLLN | RKS | NLVT |
| | 421B1 | SGFNIEDTYIHW | ID | TDYD | (SEQ | SSQSLLNGRTRR | GRTR | (SEQ | (SEQ |
| | 0C12D | VKQRPARGLEWV | NO: | PKFQ | ID | NYLAWYQQKPGQ | RNYL | ID | ID |
| | 2-AB1 | GRIDPANGNTDY | 41) | G | NO: | SPKLLIYWASTR | A | NO: | NO: |
| | | DPKFQGKATMIT | | (SEQ | 43) | KSGVPDRFTGSG | (SEQ | 46) | 47) |
| | | DTSSNTVYLQVS | | ID | | SGTDFTLTITSV | ID | | |
| | | SLTSEDTAVYFC | | NO: | | QAEDLAVYYCKQ | NO: | | |
| | | GRHDGYDMDYWG | | 42) | | SYNLVTFGAGTK | 45) | | |
| | | QGTSVTVSS | | | | LELK | | | |
| | | (SEQ | | | | (SEQ | | | |
| | | ID | | | | ID | | | |
| | | NO: | | | | NO: | | | |
| | | 40) | | | | 44) | | | |
| 417E12A8 | ACI- | QVQLQQPGAELV | SSWIT | NIYP | KDFV | DIVMTQSQKFMS | KASQ | SASY | QQYN |
| | 8016- | RPGASVILSCKA | (SEQ | SDSY | YDAW | TSVGDRVTVTCK | NVGT | RNS | NYPL |
| | 417E1 | SGYTFTSSWITW | ID | TNSN | YFDV | ASQNVGTNVAWY | NVA | (SEQ | T |
| | 2A8- | VKQRPGQGLEWI | NO: | QKFK | (SEQ | QQRPGQSPKPLI | (SEQ | ID | (SEQ |
| | AB1 | GNIYPSDSYTNS | 51) | D | ID | YSASYRNSGVPD | ID | NO: | ID |
| | | NQKFKDKATLTV | | (SEQ | NO: | RFTGSGSGTDFT | NO: | 56) | NO: |
| | | DKSSSTAYMHLI | | ID | 53) | LTITNVQSEDLA | 55) | | 57) |
| | | SPTSEDSAVYYC | | NO: | | EYFCQQYNNYPL | | | |
| | | TRKDFVYDAWYF | | 52) | | TFGAGTKLELK | | | |
| | | DVWGAGTTVTVS | | | | (SEQ | | | |
| | | S | | | | ID | | | |
| | | (SEQ | | | | NO: | | | |
| | | ID | | | | 54) | | | |
| | | NO: | | | | | | | |
| | | 50) | | | | | | | |
| 413G10A5 | ACI- | QVQLKESGPGLV | SYGVH | VIWA | WDYV | DVVMTQTPLSLP | RSSQ | KVSN | SQST |
| | 8016- | APSQSLSITCTV | (SEQ | GGST | NSYE | VSLGDQASISCR | SLVH | RFS | HVPY |
| | 413G1 | SGFSLTSYGVHW | ID | NYNS | AMDY | SSQSLVHSNGNT | SNGN | (SEQ | T |
| | 0A5- | VRQPPGKGLEWL | NO: | ALMS | (SEQ | YLHWYLQKPGQS | TYLH | ID | (SEQ |
| | AB1 | GVIWAGGSTNYN | 61) | (SEQ | ID | PKLLIYKVSNRF | (SEQ | NO: | ID |
| | | SALMSRLSISKD | | ID | NO: | SGVPDRFSGSGS | ID | 66) | NO: |
| | | NSKSQVFLKMNS | | NO: | 63) | GTDFTLKISRVE | NO: | | 67) |
| | | LQTADTAMYYCA | | 62) | | AEDLGVYFCSQS | 65) | | |
| | | RWDYVNSYEAMD | | | | THVPYTFGGGTK | | | |
| | | YWGQGTSVTVSS | | | | LEIK | | | |
| | | (SEQ | | | | (SEQ | | | |
| | | ID | | | | ID | | | |
| | | NO: | | | | NO: | | | |
| | | 60) | | | | 64) | | | |
| 407E10A9 | ACI- | EVQLVESGGGFV | NYAMS | TIYS | LGGS | QAVVTQESALTT | RSST | GTDN | ALWY |
| | 8016- | KPGGSLKLSCAA | (SEQ | GGIF | Y | SPGETVTLTCRS | GAVT | RPP | NNHW |
| | 407E1 | SGFTFSNYAMSW | ID | TYYP | (SEQ | STGAVTTNNYAN | TNNY | (SEQ | V |
| | 0A9- | VRQTPEKRLEWV | NO: | DSVK | ID | WVQEKPDHFFTG | AN | ID | (SEQ |
| | AB1 | ATIYSGGIFTYY | 71) | G | NO: | LIGGTDNRPPGV | (SEQ | NO: | ID |
| | | PDSVKGRFTISR | | (SEQ | 73) | PARFSGSLIGDK | ID | 76) | NO: |
| | | DSAKNTLYLQMT | | ID | | AALTITGAQTED | NO: | | 77) |
| | | SLRSEDTAIYYC | | NO: | | EAIYFCALWYNN | 75) | | |
| | | ARLGGSYWGQGT | | 72) | | HWVFGGGTKLTV | | | |
| | | LVTVSA | | | | L | | | |
| | | (SEQ | | | | (SEQ | | | |
| | | ID | | | | ID | | | |
| | | NO: | | | | NO: | | | |
| | | 70) | | | | 74) | | | |
| 203G8B10 | ACI- | EVKLEESGGGLV | NYWMN | QIRL | GWGL | DIVMTQAAFSNP | RSSK | RVSN | AQML |
| | 8016- | HPGGSMKLSCVA | (SEQ | KSEN | DY | VTLGTSASISCR | TLLH | LAS | ELPL |
| | 203G8 | SGFTFSNYWMNW | ID | YATH | (SEQ | SSKTLLHSDGFT | SDGF | (SEQ | T |
| | B10- | VRQSPEKGLEWV | NO: | SAES | ID | YLYWYLQRPGQS | TYLY | ID | (SEQ |
| | AB1 | AQIRLKSENYAT | 81) | VKG | NO: | PQLLIHRVSNLA | (SEQ | NO: | ID |
| | | HSAESVKGRLTI | | (SEQ | 83) | SGVPNRFSGSES | ID | 86) | NO: |
| | | SRDDSKSSVYLQ | | ID | | GTDFTLRISRVE | NO: | | 87) |
| | | MNNLRAEDTGIY | | NO: | | PEDVGVYYCAQM | 85) | | |
| | | YCLAGWGLDYWG | | 82) | | LELPLTFGSGTK | | | |
| | | QGTTLTVSS | | | | LELK | | | |
| | | (SEQ | | | | (SEQ | | | |
| | | ID | | | | ID | | | |
| | | NO: | | | | NO: | | | |
| | | 80) | | | | 84) | | | |
| 401H9B7 | ACI- | QIQLVQSGPELK | KYGMN | WINT | LGYA | DIQMTQSPASLS | RASE | NANT | QYHY |
| | 8016- | KPGETVKISCKA | (SEQ | YTGE | NSFD | ATVGETVTITCR | NIYT | LVE | GSPY |
| | 401H9 | SGYTFTKYGMNW | ID | PTYS | S | ASENIYTYLAWY | YLA | (SEQ | T |
| | B7- | VKQAPGKGLKRM | NO: | DDFK | (SEQ | QQKQGKSPQLLV | (SEQ | ID | (SEQ |
| | AB1 | GWINTYTGEPTY | 91) | G | ID | HNANTLVEGVPS | ID | NO: | ID |
| | | SDDFKGRFAFSL | | (SEQ | NO: | RFSGSGSGTQFS | NO: | 96) | NO: |
| | | ETSASTAYLQIN | | ID | 93) | LKINSLEPEDFG | 95) | | 97) |
| | | NLKNDDTATYFC | | NO: | | NYYCQYHYGSPY | | | |
| | | ARLGYANSFDSW | | 92) | | TFGGGTKLEIK | | | |
| | | GQGTTLTVSS | | | | (SEQ | | | |
| | | (SEQ | | | | ID | | | |
| | | ID | | | | NO: | | | |
| | | NO: | | | | 94) | | | |
| | | 90) | | | | | | | |
| 1112B3D7 | ACI- | QVQLQQSGAELV | SYWMH | GIDP | EEAS | DIVLTQSPATLS | RASQ | YTSQ | QQSN |
| | 8016- | RPGTSVKLSCKA | (SEQ | SDSY | TVVA | VTPGDSVSLSCR | SISN | SIS | NWPY |
| | 1112B | SGYTFTSYWMHW | ID | TKYN | HWYF | ASQSISNNLHWY | NLH | (SEQ | T |
| | 3D7- | VKQRPGQGLEWI | NO: | QKFK | DV | QQKSHESPRLLI | (SEQ | ID | (SEQ |
| | AB1 | GGIDPSDSYTKY | 111) | A | (SEQ | KYTSQSISGIPS | ID | NO: | ID |
| | | NQKFKAKATLTA | | (SEQ | ID | RFSGSGSGTDYT | NO: | 116) | NO: |
| | | DTSSSTAYMQLS | | ID | NO: | LSINSVETEDFG | 115) | | 117) |
| | | SLTSEDSAIFYC | | NO: | 113) | MYFCQQSNNWPY | | | |
| | | AREEASTVVAHW | | 112) | | TFGGGTKLEIK | | | |
| | | YFDVWGTGTTVT | | | | (SEQ | | | |
| | | VSS | | | | ID | | | |
| | | (SEQ | | | | NO: | | | |
| | | ID | | | | 114) | | | |
| | | NO: | | | | | | | |
| | | 110) | | | | | | | |
| 2221B7F1 | ACI- | EVQLVESGGGLV | DSYMY | SISD | GLDY | DIKMTQSPSSMY | KASQ | RANR | LQYA |
| | 8018- | KPGGSLKLSCAA | (SEQ | GGRY | (SEQ | ASLGERVTITCK | DINS | LVD | ESTW |
| | 2221B | SGFTFSDSYMYW | ID | TYYS | ID | ASQDINSYLSWF | YLS | (SEQ | T |
| | 7F1- | LRQTPEKRLEWV | NO: | DSVK | NO: | QQKPGKSPKTLI | (SEQ | ID | (SEQ |
| | AB1 | ASISDGGRYTYY | 121) | GS | 123) | YRANRLVDGVPS | ID | NO: | ID |
| | | SDSVKGRFTISR | | (SEQ | | RFSGSGSGQDYS | NO: | 126) | NO: |
| | | DNVKNNLYLQMT | | ID | | LTISSLEYEDMG | 125) | | 127) |
| | | SLRSEDTAIYYC | | NO: | | IYYCLQYAESTW | | | |
| | | ARGLDYWGQGTT | | 122) | | TFGGGTKLEIK | | | |
| | | LTVSS | | | | (SEQ | | | |
| | | (SEQ | | | | ID | | | |
| | | ID | | | | NO: | | | |
| | | NO: | | | | 124) | | | |
| | | 120) | | | | | | | |
| 2314F6H11 | ACI- | QVQVQQSGADLA | NYGIN | EIYP | SSFG | DVVMTQTPLSLP | RSSQ | KVSN | SQST |
| | 8019- | RPGASVKLSCKA | (SEQ | KNID | FSGN | VSLGDQASISCR | NLIH | RFSS | HVPW |
| | 2314F | SGYTFTNYGINW | ID | IYYN | YAMD | SSQNLIHSNGIT | SNGI | EQID | T |
| | 6H11- | VKQRTGQGLEWI | NO: | EKFK | Y | YLHWYLQKPGQS | TYLH | NO: | (SEQ |
| | AB1 | GEIYPKNIDIYY | 131) | G | (SEQ | PKLLIYKVSNRF | (SEQ | 66) | ID |
| | | NEKFKGKATLTA | | (SEQ | ID | SGVPHRFSGSGS | ID | | NO: |
| | | DKSSSTAYMELR | | ID | NO: | GTDFTLKISRVE | NO: | | 137) |
| | | SLTSEDSAVYFC | | NO: | 133) | AEDLGVYFCSQS | 135) | | |
| | | GRSSFGFSGNYA | | 132) | | THVPWTFGGGTK | | | |
| | | MDYWGQGTSVTV | | | | LEIK | | | |
| | | SS | | | | (SEQ | | | |
| | | (SEQ | | | | ID | | | |
| | | ID | | | | NO: | | | |
| | | NO: | | | | 134) | | | |
| | | 130) | | | | | | | |
| 207E8B2 | ACI- | QVQLQQSGAELA | SYWMH | YINP | PFDY | DVLMTQTPLSLP | RSSQ | KVSN | FQDS |
| | 8016- | KPGASVKLSCKA | (SEQ | SSGY | DGFD | VSLGDQASISCR | SIVH | RFS | HFPF |
| | 207E8 | SGYTFTSYWMHW | ID | TKCN | Y | SSQSIVHSNGNT | SNGN | (SEQ | T |
| | B2- | VKQRPGQGLEWI | NO: | QKFK | (SEQ | YLEWYLQKPGQS | TYLE | ID | (SEQ |
| | AB1 | GYINPSSGYTKC | 111) | D | ID | PKLLIYKVSNRF | (SEQ | NO: | ID |
| | | NQKFKDKATLTA | | (SEQ | NO: | SGVPDRFSGSGS | ID | 66) | NO: |
| | | DKSSSTAYMQLS | | ID | 143) | GTVFTLKISRVE | NO: | | 147) |
| | | SLTYEDSAVYYC | | NO: | | AEDLGVYYCFQD | 145) | | |
| | | VFPFDYDGFDYW | | 142) | | SHFPFTFGSGTK | | | |
| | | GQGTTLTVSS | | | | LEIK | | | |
| | | (SEQ | | | | (SEQ | | | |
| | | ID | | | | ID | | | |
| | | NO: | | | | NO: | | | |
| | | 140) | | | | 144) | | | |
| 936.2A1B12 | ACI- | EVQLQQSGAELV | TYGLT | YIYV | LGLR | AIQMTQTTSSLS | RASQ | FTSG | QQSI |
| | 8016- | RPGSSVKMSCET | (SEQ | GNGY | RHFD | ASLGDRVTISCR | DISN | LRS | LLPW |
| | 2A1B1 | SGYTFTTYGLTW | ID | TEYN | Y | ASQDISNYLHWY | YLH | (SEQ | T |
| | 2-AB1 | VKQRPGQGLEWI | NO: | EKFK | (SEQ | QQKPDGTVKLLI | (SEQ | ID | (SEQ |
| | | GYIYVGNGYTEY | 151) | D | ID | FFTSGLRSGVPS | ID | NO: | ID |
| | | NEKFKDKATLTS | | (SEQ | NO: | RFSGSGSGTDYS | NO: | 156) | NO: |
| | | DTSSSIVYMQLS | | ID | 153) | LTISNLEQEDIA | 155) | | 157) |
| | | SLTSEDSAIYFC | | NO: | | TYFCQQSILLPW | | | |
| | | ARLGLRRHFDYW | | 152) | | TFGGGTKLEIK | | | |
| | | GQGTTLTVSS | | | | (SEQ | | | |
| | | (SEQ | | | | ID | | | |
| | | ID | | | | NO: | | | |
| | | NO: | | | | 154) | | | |
| | | 150) | | | | | | | |
| 936.17H1G2 | ACI- | EVQLQQSGAELV | SYGIS | YIYI | IHRR | DIQMTQTTSSLS | SASQ | YTSS | QQYD |
| | 8016- | RPGSSVKMSCKT | (SEQ | RNGY | FFDY | ASLGDRVTISCS | GISN | LHS | RLPW |
| | 17H1G | SGFTFTSYGISW | ID | TEYN | (SEQ | ASQGISNYLNWF | YLN | (SEQ | T |
| | 2-AB1 | VIQRPEQGLEWI | NO: | EKFK | ID | QQKPNGTVRLLI | (SEQ | ID | (SEQ |
| | | GYIYIRNGYTEY | 161) | G | NO: | YYTSSLHSGVPS | ID | NO: | ID |
| | | NEKFKGKATLTS | | (SEQ | 163) | RFSGSGSGTDYS | NO: | 166) | NO: |
| | | DTSSNTAYMQLS | | ID | | LTISNLEPEDIA | 165) | | 167) |
| | | NLASEDSAIYFC | | NO: | | TYYCQQYDRLPW | | | |
| | | VRIHRRFFDYWG | | 162) | | TFGGGTKLEIK | | | |
| | | QGTTLTVSS | | | | (SEQ | | | |
| | | (SEQ | | | | ID | | | |
| | | ID | | | | NO: | | | |
| | | NO: | | | | 164) | | | |
| | | 160) | | | | | | | |
| 936.18F4C12 | ACI- | EVQLQQSGAELV | SYGMS | YIYV | LNRG | DIVLTQSPASLA | RASQ | FASN | QHSW |
| | 8016- | RPGSSVKMTCKT | (SEQ | GNGY | RALD | VSLGQRATISCR | SVST | LES | DLPY |
| | 18F4C | SGYSFSSYGMSW | ID | TEYN | F | ASQSVSTSGHSY | SGHS | (SEQ | T |
| | 12- | VKQRPGQGLEWI | NO: | EKFK | (SEQ | MHWYQQKPRQSP | YMH | ID | (SEQ |
| | AB1 | GYIYVGNGYTEY | 171) | G | ID | KLLIHFASNLES | (SEQ | NO: | ID |
| | | NEKFKGKATLTS | | (SEQ | NO: | GVPARFTGSGSG | ID | 176) | NO: |
| | | DTSSSTVYMDFR | | ID | 173) | TDFTLNIHPVEE | NO: | | 177) |
| | | SLTSEDSAIYFC | | NO: | | EDAATYYCQHSW | 175) | | |
| | | ARLNRGRALDFW | | 172) | | DLPYTFGGGTKL | | | |
| | | GQGTTLTVSS | | | | EIK | | | |
| | | (SEQ | | | | (SEQ | | | |
| | | ID | | | | ID | | | |
| | | NO: | | | | NO: | | | |
| | | 170) | | | | 174) | | | |
| 936.23E5F7 | ACI- | QVQLQQSEAELA | SYGIF | EIYP | QRDD | QIVLTQSPAIMS | SVSS | STSN | QQRS |
| | 8016- | RPGASVKLSCKA | (SEQ | RSGN | Y | ASPGEKVTITCS | SVSY | LAS | SYPF |
| | 23E5F | SGYTFTSYGIFW | ID | TYYN | (SEQ | VSSSVSYMYWFQ | MY | (SEQ | T(SEGI |
| | 7-AB1 | VKQRTGQGLEWI | NO: | ENFR | ID | QKPGTSPKVWIY | (SEQ | ID | DNO: |
| | | GEIYPRSGNTYY | 181) | D | NO: | STSNLASGVPAR | ID | NO: | 187) |
| | | NENFRDKATLTA | | (SEQ | 183) | FSGRGSGTSYSL | NO: | 186) | |
| | | DKSSSTAYMELR | | ID | | TISRMEAEDAAT | 185) | | |
| | | SLTSEDSAVYFC | | NO: | | YYCQQRSSYPFT | | | |
| | | ARQRDDYWGQGT | | 182) | | FGSGTKLEIK | | | |
| | | TLTVSS | | | | (SEQ | | | |
| | | (SEQ | | | | ID | | | |
| | | ID | | | | NO: | | | |
| | | NO: | | | | 184) | | | |
| | | 180) | | | | | | | |
| 936.26A1G2 | ACI- | QVQLQQSGAELA | TYGIN | KIYP | SPYA | DIKMTQSPSSMY | KASQ | YATS | LQHG |
| | 8016- | RPGASVKLSCKA | (SEQ | RSGN | FYSS | ASLGERVTITCK | DIKS | LAD | ESPF |
| | 26A1G | SGYNFTTYGINW | ID | THNN | SYWY | ASQDIKSYLSWY | YLS | (SEQ | T |
| | 2-AB1 | VKQRTGQGLEWI | NO: | ENFK | FDV | QQKPWKSPKTLI | (SEQ | ID | (SEQ |
| | | GKIYPRSGNTHN | 191) | G | (SEQ | YYATSLADGVPS | ID | NO: | ID |
| | | NENFKGKATLTA | | (SEQ | ID | RFSGSGSGQDYS | NO: | 196) | NO: |
| | | DTSSSTAYMDLR | | ID | NO: | LTISSLESDDTA | 195) | | 197) |
| | | SLTSEDSAVYFC | | NO: | 193) | TYYCLQHGESPF | | | |
| | | ARSPYAFYSSSY | | 192) | | TFGSGTKLEIK | | | |
| | | WYFDVWGTGTTV | | | | (SEQ | | | |
| | | TVSS | | | | ID | | | |
| | | (SEQ | | | | NO: | | | |
| | | ID | | | | 194) | | | |
| | | NO: | | | | | | | |
| | | 190) | | | | | | | |
| 936.32B6C7 | ACI- | QVQLQQSDAELV | DQTIH | YIYP | PLYS | DIQMNQSPSSLS | HASQ | KSSN | QQGQ |
| | 8016- | KPGASVKISCKI | (SEQ | GDGS | YASY | ASLGDTITITCH | NINV | LHT | SYPL |
| | 32B6C | SGYTFTDQTIHW | ID | AMYN | YWFA | ASQNINVWLSWY | WLS | (SEQ | T |
| | 7-AB1 | MNQRPEQGLEWI | NO: | EKFR | Y | QRKPGNIPKLLI | (SEQ | ID | (SEQ |
| | | GYIYPGDGSAMY | 201) | G | (SEQ | YKSSNLHTGVPS | ID | NO: | ID |
| | | NEKFRGKATLTA | | (SEQ | ID | RFSGSGSGTGFT | NO: | 206) | NO: |
| | | DKSSSTAYMQLN | | ID | NO: | LTISSLQPEDIA | 205) | | 207) |
| | | SLTSEDSAVYFC | | NO: | 203) | TYYCQQGQSYPL | | | |
| | | ARPLYSYASYYW | | 202) | | TFGGGTKLEIK | | | |
| | | FAYWGQGTLVTV | | | | (SEQ | | | |
| | | SA | | | | ID | | | |
| | | (SEQ | | | | NO: | | | |
| | | ID | | | | 204) | | | |
| | | NO: | | | | | | | |
| | | 200) | | | | | | | |
| 936.22D3A6 | ACI- | QAQLQQSGTELA | IYGIN | EIYP | SREW | QIVLTQSPAIMS | SATS | STSN | QQRS |
| | 8016- | RPGSSVKLSCKA | (SEQ | RSGN | EGVF | AFPGEKVTITCS | SVSY | LAS | RYPF |
| | 22D3A | SGFTFTIYGINW | ID | TYYD | (SEQ | ATSSVSYMHWFQ | MH | (SEQ | T(SEQ |
| | 6- | VKQRTGQGLEWI | NO: | EKFK | ID | QKPGTSPKLWIY | (SEQ | ID | Ď |
| | AB1_h | GEIYPRSGNTYY | 211) | G | NO: | STSNLASGVPAR | ID | NO: | NO217) |
| | mG2c | DEKFKGKATLTA | | (SEQ | 213) | FSGSGSGTSHSL | NO: | 186) | |
| | | DKSTSTAYMELR | | ID | | TINRMEAEDAAT | 215) | | |
| | | SLTSEDSAVYFC | | NO: | | YYCQQRSRYPFT | | | |
| | | ARSREWEGVFWG | | 212) | | FGSGTKLEIK | | | |
| | | QGTLVTVSA | | | | (SEQ | | | |
| | | (SEQ | | | | ID | | | |
| | | ID | | | | NO: | | | |
| | | NO: | | | | 214) | | | |
| | | 210) | | | | | | | |
| 936.31F10C5 | ACI- | QVQLQQSEPELA | NYGVS | RIYP | FPYA | DIVMTQSHKFMS | KASQ | SSSF | QQHF |
| | 8016- | RPGASVKLSCTA | (SEQ | RSGN | FSGN | TSIGDRVSITCK | DVSA | RYT | DTPY |
| | 31F10 | SGYTFINYGVSW | ID | TRFN | SYWY | ASQDVSAAVAWY | AVA | (SEQ | T |
| | C5- | VRQRTGQGLEWI | NO: | EKFQ | FDV | QQKPGQSPKLLI | (SEQ | ID | (SEQ |
| | AB1 | GRIYPRSGNTRF | 221) | G | (SEQ | YSSSFRYTGVPD | ID | NO: | ID |
| | | NEKFQGKATLTA | | (SEQ | ID | RFTGSASGTDFT | NO: | 226) | NO: |
| | | DTSSSTAYMELR | | ID | NO: | FTITTVQAEDLA | 225) | | 227) |
| | | SLTSEDSAVYFC | | NO: | 223) | VYYCQQHFDTPY | | | |
| | | TRFPYAFSGNSY | | 222) | | TFGGGTKLEIK | | | |
| | | WYFDVWGTGTTV | | | | (SEQ | | | |
| | | TVSS | | | | ID | | | |
| | | (SEQ | | | | NO: | | | |
| | | ID | | | | 224) | | | |
| | | NO: | | | | | | | |
| | | 220) | | | | | | | |
| 936.19E6D4 | ACI- | EVKLVESGGGLV | DYYMY | YISS | EGFY | DIVLSQSPATLS | RASQ | SASQ | QQSD |
| | 8016- | QPGGSLKLSCAA | (SEQ | GGDA | YYGL | VTPGDSVSLSCR | SIGD | SIS | TWPL |
| | 19E6D | SGFTFSDYYMYW | ID | PFYS | YGMD | ASQSIGDNLHWY | NLH | (SEQ | T |
| | 4-AB1 | VRQNAEKRLEWV | NO: | DNLK | Y | QQKSHESPRLII | (SEQ | ID | (SEQ |
| | | AYISSGGDAPFY | 231) | G | (SEQ | KSASQSISGIPS | ID | NO: | ID |
| | | SDNLKGRFTISR | | (SEQ | ID | RFSGSGSGTDFT | NO: | 236) | NO: |
| | | DNAKNTLYLQMS | | ID | NO: | LSINIVETEDFG | 235) | | 237) |
| | | RLQPEDTAMYFC | | NO: | 233) | MYFCQQSDTWPL | | | |
| | | AREGFYYYGLYG | | 232) | | TFGPGTKLELK | | | |
| | | MDYWGQGTSVTV | | | | (SEQ | | | |
| | | SS | | | | ID | | | |
| | | (SEQ | | | | NO: | | | |
| | | ID | | | | 234) | | | |
| | | NO: | | | | | | | |
| | | 230) | | | | | | | |
| 936.3E6B11 | ACI- | EVQLQQSGAELV | NYGIT | YIYV | IAQG | DIVLTQSPASSA | RASQ | YAST | QHSW |
| | 8016- | RPGSSVKMSCKT | (SEQ | GNGY | RCFD | VSLGQRATISCR | SVST | LES | ELPY |
| | 3E6B1 | SGYTFTNYGITW | ID | TEYN | Y | ASQSVSTSSYSY | SSYS | (SEQ | M |
| | 1-AB1 | VKQRPGQGLEWI | NO: | EKFK | (SEQ | MHWYQQKPGQPP | YMH | ID | (SEQ |
| | | GYIYVGNGYTEY | 241) | D | ID | KLLIKYASTLES | (SEQ | NO: | ID |
| | | NEKFKDKAALTS | | (SEQ | NO: | GVPARFSGSGSG | ID | 246) | NO: |
| | | DTSSSTAYMQLT | | ID | 243) | TDFTLNIHPVEE | NO: | | 247) |
| | | SLTSDDSTIYFC | | NO: | | EDTATYYCQHSW | 245) | | |
| | | ARIAQGRCFDYW | | 152) | | ELPYMFGGGTKL | | | |
| | | GQGTTLSVSS | | | | EIK | | | |
| | | (SEQ | | | | (SEQ | | | |
| | | ID | | | | ID | | | |
| | | NO: | | | | NO: | | | |
| | | 240) | | | | 244) | | | |
| 936.11A3F3 | ACI- | QVQLQQSGAELA | SFGLS | EIYP | YFTK | DIQMTQSPASLA | RASE | YANS | KQAY |
| | 8016- | RPGTSVKLSCKA | (SEQ | RIDN | GNYF | ASVGETVTITCR | NIYY | LED | DVPF |
| | 11A3F | SGYTFISFGLSW | ID | PYYN | DY | ASENIYYSLAWY | SLA | (SEQ | T |
| | 3-AB1 | VKQRTGQGLEWI | NO: | EKFK | (SEQ | QQKQGKSPQVLI | (SEQ | ID | (SEQ |
| | | GEIYPRIDNPYY | 251) | G | ID | YYANSLEDGVPS | ID | NO: | ID |
| | | NEKFKGKAILTA | | (SEQ | NO: | RFSGSGSGTQYS | NO: | 256) | NO: |
| | | DKSSSTAYMELR | | ID | 253) | LKINSMQPEDTA | 255) | | 257) |
| | | SLTSEDSAVYFC | | NO: | | TYFCKQAYDVPF | | | |
| | | ARYFTKGNYFDY | | 252) | | TFGTGTKLELK | | | |
| | | WGQGTTLTVSS | | | | (SEQ | | | |
| | | (SEQ | | | | ID | | | |
| | | ID | | | | NO: | | | |
| | | NO: | | | | 254) | | | |
| | | 250) | | | | | | | |
| 936.14G5B8 | ACI- | EVQLQESGPELV | DHYMN | DINP | RRAH | DIVMTQSQKFMS | KASQ | SASI | QQYS |
| | 8016- | KPGSSVKISCKA | (SEQ | NNGG | YYDG | TSVGDRVSLTCK | IVGT | RYT | NYPT |
| | 14G5B | SGYTFADHYMNW | ID | SVYN | PYFD | ASQIVGTAVAWY | AVA | (SEQ | (SEQ |
| | 8-AB1 | VKQSHGKSLEWI | NO: | QKFK | Y | QQKPGQSPKLLI | (SEQ | ID | ID |
| | | GDINPNNGGSVY | 261) | G | (SEQ | YSASIRYTGVPD | ID | NO: | NO: |
| | | NQKFKGKATLTV | | (SEQ | ID | RFTGSGSGTDFT | NO: | 266) | 267) |
| | | DKSSSTAYMELR | | ID | NO: | LTISNMQSEDLA | 265) | | |
| | | SLTSEDSAVYFC | | NO: | 263) | DYFCQQYSNYPT | | | |
| | | ARRRAHYYDGPY | | 262) | | FGAGTKLELK | | | |
| | | FDYWGQGTTLTV | | | | (SEQ | | | |
| | | SS | | | | ID | | | |
| | | (SEQ | | | | NO: | | | |
| | | ID | | | | 264) | | | |
| | | NO: | | | | | | | |
| | | 260) | | | | | | | |
| 936.27A1G4 | ACI- | QVQLQQSDAELV | DHTIH | YIYP | PLYS | DIQMNQSPSSLS | HASQ | KASN | QQGQ |
| | 8016- | KPGASVKISCKV | (SEQ | GDAS | YASY | ASLGDTITITCH | NINV | LHT | SYPL |
| | 27A1G | SGYTFTDHTIHW | ID | SYYN | YWFA | ASQNINVWLTWY | WLT | (SEQ | T |
| | 4-AB1 | MRQRPEQGLEWI | NO: | EKFK | Y | QQKPGNIPKLLI | (SEQ | ID | (SEQ |
| | | GYIYPGDASSYY | 271) | G | (SEQ | YKASNLHTDVPS | ID | NO: | ID |
| | | NEKFKGKATLTA | | (SEQ | ID | RFSGSGSGTGFT | NO: | 276) | NO: |
| | | DQSSNTAYIQVN | | ID | NO: | LTISSLQPEDIA | 275) | | 207) |
| | | SLTSEDSAVYFC | | NO: | 203) | TYYCQQGQSYPL | | | |
| | | ARPLYSYASYYW | | 272) | | TFGGGTKLEIK | | | |
| | | FAYWGQGTLVTV | | | | (SEQ | | | |
| | | SA | | | | ID | | | |
| | | (SEQ | | | | NO: | | | |
| | | ID | | | | 274) | | | |
| | | NO: | | | | | | | |
| | | 270) | | | | | | | |
| 936.29C5E11 | ACI- | EVQLQQSGAELV | NNGIT | YIYV | LDRG | DIVLTQSPASLA | RASK | LASN | QHSW |
| | 8016- | RPGSSVKMSCRT | (SEQ | GNGY | RALD | VSLGQRATISCR | SVSS | LES | ELPY |
| | 29C5E | SGYTFTNNGITW | ID | TEYN | Y | ASKSVSSSAYSY | SAYS | (SEQ | T |
| | 11- | VKQRPGQGLEWI | NO: | EKFK | (SEQ | MHWYQQKPGQPP | YMH | ID | (SEQ |
| | AB1 | GYIYVGNGYTEY | 281) | D | ID | KLLIHLASNLES | (SEQ | NO: | ID |
| | | NEKFKDKATLTS | | (SEQ | NO: | GVPARFSGSGSG | ID | 286) | NO: |
| | | DTASSTVYMQLN | | ID | 283) | TDFTLNIHPVEE | NO: | | 287) |
| | | SLTSEDSAIYFC | | NO: | | EDAATYYCQHSW | 285) | | |
| | | ARLDRGRALDYW | | 152) | | ELPYTFGGGTKL | | | |
| | | AQGTTLTVSS | | | | EIK | | | |
| | | (SEQ | | | | (SEQ | | | |
| | | ID | | | | ID | | | |
| | | NO: | | | | NO: | | | |
| | | 280) | | | | 284) | | | |
| 936.7G3B5 | ACI- | EVQLQQSVAELV | NTYMH | RIDP | WGFD | DVVLTQSPATLS | RASQ | YASQ | QQSN |
| | 8016- | RPGASVKLSCTA | (SEQ | ANGN | KDGV | VTPGDSVSLSCR | SIYN | SISS | SWPL |
| | 7G3B5 | SGFNIKNTYMHW | ID | TKYV | VYAM | ASQSIYNNLHWY | NLH | EQID | T |
| | -AB1 | VKQRPEQGLEWI | NO: | PKFQ | DY | QQKSHESPRLLV | (SEQ | NO: | (SEQ |
| | | GRIDPANGNTKY | 291) | G | (SEQ | KYASQSISGIPS | ID | 26) | ID |
| | | VPKFQGKATITA | | (SEQ | ID | RFSGSGSGTDFT | NO: | | NO: |
| | | DTSSKTAYLQLS | | ID | NO: | LSINSVETEDFG | 295) | | 297) |
| | | SLTSEDTAIYYC | | NO: | 293) | MYFCQQSNSWPL | | | |
| | | TRWGFDKDGVVY | | 292) | | TFGAGTKLELK | | | |
| | | AMDYWGQGTSVT | | | | (SEQ | | | |
| | | VSS | | | | ID | | | |
| | | (SEQ | | | | NO: | | | |
| | | ID | | | | 294) | | | |
| | | NO: | | | | | | | |
| | | 290) | | | | | | | |
| 2504F3D9 | ACI- | DVKLVESGEGLV | NYAMS | YISS | QLRP | DIKMTQSPSSMY | KASQ | RANR | LQYY |
| | 8016- | KPGGSLKLSCAV | (SEQ | GGDF | EAWF | ASLGERVTITCK | DIKN | LVD | EFPY |
| | 2504F | SGFTFSNYAMSW | ID | IYYR | AY | ASQDIKNYLSWF | YLS | (SEQ | T |
| | 3D9- | VRQTPEKRLEWV | NO: | DTVK | (SEQ | QQIPGKSPKTLI | (SEQ | ID | (SEQ |
| | AB1 | AYISSGGDFIYY | 71) | G | ID | YRANRLVDGVPS | ID | NO: | ID |
| | | RDTVKGRFTISR | | (SEQ | NO: | RFSGSGSGQDYS | NO: | 126) | NO: |
| | | DNARNTLYLQMS | | ID | 303) | LTISSLEYEDMG | 305) | | 307) |
| | | SLKSEDTAMYYC | | NO: | | IYYCLQYYEFPY | | | |
| | | ILQLRPEAWFAY | | 302) | | TFGGGTKLEIK | | | |
| | | WGQGTLVTVSA | | | | (SEQ | | | |
| | | (SEQ | | | | ID | | | |
| | | ID | | | | NO: | | | |
| | | NO: | | | | 304) | | | |
| | | 300) | | | | | | | |
| 2516A8C6 | ACI- | QVQLQQSGAELA | SYGIS | KIYP | RDY | QIVLTQSPVIMS | SASS | STSN | QQRS |
| | 8016- | RPGASVKLSCKA | (SEQ | RSGN | | ASPGEKVTITCS | SVSY | LAS | RYPW |
| | 2516A | SGHTFTSYGISW | ID | TYYD | | ASSSVSYMYWFQ | MY | (SEQ | T |
| | 8C6- | VKQRTGQGLEWI | NO: | EKFK | | QKPGTSPKFWIY | (SEQ | ID | (SEQ |
| | AB1 | GKIYPRSGNTYY | 161) | G | | STSNLASGVPAR | ID | NO: | ID |
| | | DEKFKGKATLTA | | (SEQ | | FSGSGSGTSYSL | NO: | 186) | NO: |
| | | DKPSTTAYMELR | | ID | | TISRMEAEDAAT | 315) | | 317) |
| | | SLTSEDSAVYFC | | NO: | | YYCQQRSRYPWT | | | |
| | | ATRDYWGQGTTL | | 312) | | FGGGTKLEIK | | | |
| | | TVSS | | | | (SEQ | | | |
| | | (SEQ | | | | ID | | | |
| | | ID | | | | NO: | | | |
| | | NO: | | | | 314) | | | |
| | | 310) | | | | | | | |
| 2602H6F10 | ACI- | QVQLQQSGAELA | SYGIS | QIYL | YYGS | DIQMNQSPSSLS | HASQ | QASN | QQGQ |
| | 8016- | RPGASVKLSCKA | (SEQ | RSSN | SSDY | ASLGDTITINCH | NINV | LQT | RYPW |
| | 2602H | SGFSFTSYGISW | ID | TYYN | (SEQ | ASQNINVWLSWY | WLS | (SEQ | T |
| | 6F10- | VKQRTGQGLEWI | NO: | EKFK | ID | QQKPGNIPKVLI | (SEQ | ID | (SEQ |
| | AB1 | GQIYLRSSNTYY | 161) | D | NO: | YQASNLQTGVPT | ID | NO: | ID |
| | | NEKFKDKATLTA | | (SEQ | 323) | RFSGSGSGTGFT | NO: | 326) | NO: |
| | | DKSSSTVYMELR | | ID | | LTISRLQPEDIA | 205) | | 327) |
| | | SLTSEDSAVYFC | | NO: | | TYYCQQGQRYPW | | | |
| | | ARYYGSSSDYWG | | 322) | | TFGGGTKLEIK | | | |
| | | QGTTLTVSS | | | | (SEQ | | | |
| | | (SEQ | | | | ID | | | |
| | | ID | | | | NO: | | | |
| | | NO: | | | | 324) | | | |
| | | 320) | | | | | | | |
| 2609F4A9 | ACI- | QVQLQQSDAELV | DHSI | YMYP | PYSF | DIQMNQSPSSLS | HASQ | KASK | QQGQ |
| | 8016- | KPGASVKISCKV | H | GDGS | GSSY | ASLGDTITITCH | NINV | LHT | SYPY |
| | 2609F | SGYALTDHSIHW | (SEQ | ANYN | DYFG | ASQNINVWLNWY | WLN | (SEQ | T |
| | 4A9- | MKQRPEQGLAWI | ID | DKFR | Y | QQKPGNIPKLLI | (SEQ | ID | (SEQ |
| | AB1 | GYMYPGDGSANY | NO: | G | (SEQ | YKASKLHTGVSS | ID | NO: | ID |
| | | NDKFRGKATLTA | 331) | (SEQ | ID | SFSGSGSGTGFT | NO: | 336) | NO: |
| | | DISSSTAYMHLN | | ID | NO: | LTISSLQPEDIG | 335) | | 337) |
| | | SLTSEDSAVYFC | | NO: | 333) | TYYCQQGQSYPY | | | |
| | | TRPYSFGSSYDY | | 332) | | TFGGGTKLEIK | | | |
| | | FGYWGQGTTLTV | | | | (SEQ | | | |
| | | SS | | | | ID | | | |
| | | (SEQ | | | | NO: | | | |
| | | ID | | | | 334) | | | |
| | | NO: | | | | | | | |
| | | 330) | | | | | | | |
| 2610H7D3 | ACI- | QVQLQQSGVELA | SYGI | QIYL | YYGS | DIQMNQSPSSLS | HASQ | QASN | QQGQ |
| | 8016- | RPGASVKLSCKA | S | RSSN | SSDY | ASLGDTITINCH | NINV | LQT | SYPW |
| | 2610H | SGFSFTSYGISW | (SEQ | TYYS | (SEQ | ASQNINVWLSWY | WLS | (SEQ | T |
| | 7D3- | VKQKTGQGLEWI | ID | EKFK | ID | QQKPGNIPKVLI | (SEQ | ID | (SEQ |
| | AB1 | GQIYLRSSNTYY | NO: | D | NO: | NQASNLQTDVPT | ID | NO: | ID |
| | | SEKFKDRATLTA | 161) | (SEQ | 323) | RFSGSGSGTDFK | NO: | 326) | NO: |
| | | DKSSSTVYMELR | | ID | | LIISRLQSEDIA | 205) | | 347) |
| | | SLTSEDSAVYFC | | NO: | | TYYCQQGQSYPW | | | |
| | | ARYYGSSSDYWG | | 342) | | TFGGGTKLEIK | | | |
| | | QGTTLTVSS | | | | (SEQ | | | |
| | | (SEQ | | | | ID | | | |
| | | ID | | | | NO: | | | |
| | | NO: | | | | 344) | | | |
| | | 340) | | | | | | | |
| 2614C3B2 | ACI- | QVQLQQSDAELV | DHSI | YMYP | PYYF | DIQMNQSPSSLS | HASQ | KASK | QQGQ |
| | 8016- | KPGASVKISCKV | H | GDGS | GSSY | ASLGDTITITCH | NINV | LHT | SYPY |
| | 2614C | SGYALTDHSIHW | (SEQ | ANYN | DYFG | ASQNINVWLSWY | WLS | (SEQ | T |
| | 3B2- | MKQRPEQGLAWI | ID | DKFK | Y | QQKPGNVPKLLI | (SEQ | ID | (SEQ |
| | AB1 | GYMYPGDGSANY | NO: | G | (SEQ | YKASKLHTGVSS | ID | NO: | ID |
| | | NDKFKGKATLTA | 331) | (SEQ | ID | NFSGSGSGTGFT | NO: | 336) | NO: |
| | | DISSSTAYMQLN | | ID | NO: | LTISSLQPEDIG | 205) | | 337) |
| | | SLTSEDSAVYFC | | NO: | 353) | TYYCQQGQSYPY | | | |
| | | TRPYYFGSSYDY | | 352) | | TFGGGTKLEIK | | | |
| | | FGYWGQGTTLTV | | | | (SEQ | | | |
| | | SS | | | | ID | | | |
| | | (SEQ | | | | NO: | | | |
| | | ID | | | | 354) | | | |
| | | NO: | | | | | | | |
| | | 350) | | | | | | | |
| 2617C3A8 | ACI- | QVQLQQSGVELA | SYSI | VIYP | RDY | QIVLTQSPAIMS | SASS | STSN | QQRS |
| | 8016- | RPGASVKLSCKA | T | RSDN | | ASPGEKVTITCS | SVSY | LAS | NYPF |
| | 2617C | SGSTFTSYSITW | (SEQ | TYYD | | ASSSVSYMYWFQ | MY | (SEQ | T |
| | 3A8- | VKQRTGQGLEWI | ID | EKFK | | QKPGTSPKFWIY | (SEQ | ID | (SEQ |
| | AB1 | GVIYPRSDNTYY | NO: | G | | STSNLASGVPAR | ID | NO: | ID |
| | | DEKFKGKATLTA | 361) | (SEQ | | FSGSGSGTSYSL | NO: | 186) | NO: |
| | | DKSSSTAYIELR | | ID | | TISRMEAEDAAT | 315) | | 367) |
| | | SLTSEDSAVYFC | | NO: | | YYCQQRSNYPFT | | | |
| | | ATRDYWGQGTTL | | 362) | | FGSGTKLEIK | | | |
| | | TVSS | | | | (SEQ | | | |
| | | (SEQ | | | | ID | | | |
| | | ID | | | | NO: | | | |
| | | NO: | | | | 364) | | | |
| | | 360) | | | | | | | |
| 2622E12F11 | ACI- | RVQLQQAGAELV | NYWI | RIHP | LSLY | DIVLTQSPASLA | RASK | LASD | QHSR |
| | 8016- | KPGASVKVSCKA | Y | SDSG | AMDS | VSLGQRATISCR | SVSS | LES | EFPW |
| | 2622E | SGNTFNNYWIYW | (SEQ | TNYN | (SEQ | ASKSVSSFGYNY | FGYN | (SEQ | T |
| | 12F11- | VKQRPGQGLEWI | ID | QKFE | ID | IHWYLQKPGQPP | YIH | ID | (SEQ |
| | AB1 | GRIHPSDSGTNY | NO: | G | NO: | KLLIYLASDLES | (SEQ | NO: | ID |
| | | NQKFEGKATLTV | 371) | (SEQ | 373) | GVPARFSGSGSG | ID | 376) | NO: |
| | | DKSSGTAYMQLS | | ID | | TGFTLNIHPVEE | NO: | | 377) |
| | | SLTAEDSAVYYC | | NO: | | EDAATYYCQHSR | 375) | | |
| | | ALLSLYAMDSWG | | 372) | | EFPWTFGGGTKL | | | |
| | | HGTSVTVSS | | | | EIK | | | |
| | | (SEQ | | | | (SEQ | | | |
| | | ID | | | | ID | | | |
| | | NO: | | | | NO: | | | |
| | | 370) | | | | 374) | | | |
| 2626B9D3 | ACI- | QVQLQQPGTELV | WTH | NINL | SMVP | NIVMTQYPKSMS | KASE | GASN | GQSY |
| | 8016- | KPGASVKLSCRA | (SEQ | SNGR | HFFP | LSIGERVTLRCK | NVGS | RYT | NYPL |
| | 2626B | SGWTHWVKQRPG | ID | TNYN | MDY | ASENVGSYVSWY | YVS | (SEQ | T |
| | 9D3- | QGLEWIGNINLS | NO: | EKFR | (SEQ | QQKPEQSPKLLI | (SEQ | ID | (SEQ |
| | AB1 | NGRTNYNEKFRA | 381) | A | ID | YGASNRYTGVPD | ID | NO: | ID |
| | | MATLTVDKSSST | | (SEQ | NO: | RFTGSGSATDFI | NO: | 386) | NO: |
| | | AYTQLSSLTSED | | ID | 383) | LTITSVQAEDLA | 385) | | 387) |
| | | SAVYYCANSMVP | | NO: | | DYHCGQSYNYPL | | | |
| | | HFFPMDYWGQGT | | 382) | | TFGAGTKLELK | | | |
| | | SVTVSS | | | | (SEQ | | | |
| | | (SEQ | | | | ID | | | |
| | | ID | | | | NO: | | | |
| | | NO: | | | | 384) | | | |
| | | 380) | | | | | | | |
| 2629E8D1 | ACI- | QVQLQQSDAELV | DHTI | NIYP | PYYF | DIQMNQSPSSLS | HASQ | KASN | QQGQ |
| | 8016- | KPGGSVKISCKV | H | GDGS | GSAS | ASLGDTITITCH | NINV | LHT | SYPL |
| | 2629E | SGYTFTDHTIHW | (SEQ | TMYN | YYFG | ASQNINVWLNWY | WLN | (SEQ | T |
| | 8D1- | MKQRPEQGLEWI | ID | EKFK | Y | QQKPGNIPKLLI | (SEQ | ID | (SEQ |
| | AB1 | GNIYPGDGSTMY | NO: | G | (SEQ | YKASNLHTGVPS | ID | NO: | ID |
| | | NEKFKGKATLTA | 271) | (SEQ | ID | RFSGSGSGIGFT | NO: | 276) | NO: |
| | | DKSSSTAYMHLN | | ID | NO: | LTISSLQPEDIA | 335) | | 207) |
| | | SLTSEDSAVYFC | | NO: | 393) | TYYCQQGQSYPL | | | |
| | | ARPYYFGSASYY | | 392) | | TFGGGTKLEIK | | | |
| | | FGYWGQGTTLTV | | | | (SEQ | | | |
| | | SS | | | | ID | | | |
| | | (SEQ | | | | NO: | | | |
| | | ID | | | | 394) | | | |
| | | NO: | | | | | | | |
| | | 390) | | | | | | | |

### Example 11: Humanization of Anti-ASC Mouse Monoclonal Antibody

### Design of the Humanized Variable Regions

The murine antibody, ACI-8016-32B6C7-AB1, was humanized by CDR-grafting into human framework regions. Human germline used for humanization of ACI-8016-32B6C7-AB1 was selected by sequence homology matching and taking into consideration their putative stability. Databases of human and mouse germline variable genes such as the IMGT database (Ehrenmann, F et al, (2010) Nucl. Acids Res., 38(S1): D301-D307) or IgBlast (Ye J. et al, (2013), Nucleic Acids Res. 2013 July; 41(Web Server issue): W34-W40) were used to identify the closest human variable domain subfamilies to the murine heavy and light chain variable regions (SEQ ID NO: 200 and 204 respectively).

For example, the IMGT database may be used to indicate the best sequence homology between ACI-8016-32B6C7-AB1 heavy chain variable (VH) domain and the members of the human VH domain subfamily 1. Highest homologies and identities of both CDRs and framework sequences were observed for germline sequences: IGHV1-69, IGHV1-46, IGHV1-3 or IGHV1-24, all of which have sequence identity above 60% for the whole sequence up to CDR3. Among other germline family putatively stable, IGHV5-51 has the highest sequence identity with 57%. IGHV1-69 and IGHV5-51 were selected as VH frameworks for humanization.

Using the same approach, ACI-8016-32B6C7-AB1 light chain variable (VL) domain sequence showed the best sequence homology to the members of the VL domain kappa subfamily 1. Highest homologies and identities of both CDRs and framework sequences were observed for germline sequences: IGKV1-33, IGKV1D-33, IGKV1D-16, IGKV1-39, IGKV1-12, IGKV1D-12 or IGKV1-5 all of which have sequence identity above 75% for the whole sequence up to CDR3. Among other germline family putatively stable, IGKV3-11 and IGKV3-15 had the highest sequence identity. IGKV1-5 and IGHV3-11 were elected as VL acceptor frameworks for humanization.

As starting point for the humanization process, murine CDRs were grafted on human acceptor frameworks for both VH and VL regions. To retain the conformation of CDRs, key positions in the human acceptor frameworks were modified by substituting human to mouse residues.

To identify residues that may most greatly impact CDR conformation and/or VH/VL orientation, a 3D model for the murine and the human-mouse hybrid VH-VL pair were generated by homology modelling using the A body builder server (Dunbar, J. et al (2016). Nucleic Acids Res. 44. W474-W478). Model analysis allows the selection of a subset of amino acid positions including the positions listed in Table 18.

Post translational modification sites were predicted by sequence analysis of ACI-8016-32B6C7-AB1 CDRs. In the VH domain, D54, and G55 form an isomerization site in CDR H2 exposed to solvent. Substitutions G55S or G55A were introduced in some constructs to remove the isomerization motif. In the variable light chain, an oxidation site was identified at position W32 in CDR L1 and was mutated to a tyrosine in some constructs.

TABLE 18
Backmutations introduced in the human framework (Kabat
numbering) of ACI-8016-32B6C7-AB1. At position 24, substitutions
were different between germline IGHV1-69 and IGHV5-51,
the two possible changes are indicated.

| Variable domain | Position (Kabat) | Mouse residue | Human residue |
|---|---|---|---|
| VH | 24 | I | A/G |
| VH | 30 | T | S |
| VH | 37 | M | V |
| VH | 48 | I | M |
| VH | 64 | R | Q |
| VH | 67 | V | A |
| VH | 69 | I | L |
| VH | 91 | F | Y |
| VL | 38 | R | Q |
| VL | 45 | K | R |

Substitutions from Table 18 were combined to generate the sequences listed in Table 20. The corresponding nucleic acid sequences encoding the ASC binding molecules are shown in Table 19.

Humanized heavy and light chains of ACI-8016-32B6C7-AB1 were combined in a matrix manner to produce the humanized variants listed in Tables 19 and 20.

TABLE 19
Nucleotide sequences of the humanized Heavy Chain and humanized Light Chain Variable
Domains (VH and VL)

| Antibody | | |
|---|---|---|
| name | VH sequence | VL sequence |
| ACI-8016- | CAGGTCCAGCTGCAGCAGTCTGATGCCG | GACATCCAGATGAACCAGTCTCCATCCAGC |
| 32B6C7- | AGCTTGTGAAGCCTGGCGCCTCCGTGAA | CTGTCTGCCAGCCTGGGCGACACCATCACC |
| AB1 | GATCTCCTGCAAGATCTCCGGCTACACCT | ATCACATGTCACGCCAGCCAGAACATCAAC |
| | TCACCGACCAGACCATCCACTGGATGAA | GTGTGGCTGTCCTGGTATCAGCGGAAGCC |
| | CCAGAGGCCTGAGCAGGGCCTTGAGTG | CGGCAACATCCCCAAGCTGCTGATCTACAA |
| | GATCGGCTACATCTATCCTGGCGACGGC | GTCCTCCAACCTGCACACCGGCGTGCCCTC |
| | TCCGCCATGTACAACGAGAAGTTCAGAG | TAGATTTTCCGGCTCTGGCTCTGGCACCGG |
| | GCAAGGCTACCCTGACCGCCGACAAGTC | CTTTACCCTGACAATCAGCTCCCTGCAGCC |
| | CTCTTCCACCGCTTACATGCAGCTGAACT | TGAGGATATCGCCACCTACTACTGTCAGCA |
| | CCCTGACCTCCGAGGACTCCGCCGTGTAC | GGGCCAGAGCTACCCTCTGACATTTGGCG |
| | TTTTGTGCCCGGCCTCTGTACTCCTACGC | GCGGAACAAAGCTGGAAATCAAG |
| | CTCCTACTATTGGTTCGCCTACTGGGGCC | (SEQ ID NO: 559) |
| | AGGGCACCCTGGTTACAGTTTCTGCT | |
| | (SEQ ID NO: 558) | |
| hACI-8016- | GAAGTACAGCTCGTGCAGTCGGGTGCCG | GATATTCAGATGACCCAATCCCCCTCTACC |
| 32B6C7- | AGGTCAAGAAGCCGGGCGAGAGCTTGA | CTATCCGCTTCCGTGGGGGACCGGGTGAC |
| AB1_H1L1 | AGATCTCATGTAAGATCAGCGGCTATAC | AATCACGTGCCATGCAAGCCAGAATATCA |
| | CTTCACTGATCAGACCATACACTGGATGC | ACGTTTGGCTGAGCTGGTACCAGAGAAAG |
| | ATCAGATGCCTGGGAAAGGTCTAGAATG | CCAGGAAAGGCCCCTAAACTTTTGATTTAC |
| | GATCGGCTATATTTACCCAGGAGATGGA | AAGTCATCCAACTTACACACAGGAGTCCCC |
| | TCCGCAATGTATAACGAGAAGTTTAGGG | TCGAGGTTTAGCGGCTCAGGTAGTGGCAC |
| | GACAGGCTACACTGAGTGCAGACAAATC | AGAATTCACTCTGACTATCTCTAGTCTGCA |
| | CATTAGCACTGCGTACCTGCAATGGAGTT | GCCAGATGACTTCGCCACCTACTATTGTCA |
| | CTCTCAAAGCCAGCGACACCGCTATGTAC | ACAGGGGCAGTCTTATCCTCTCACCTTTGG |
| | TTTTGCGCCAGACCCCTTTACTCTTATGCC | CCAGGGTACTAAACTCGAGATAAAG |
| | TCTTACTACTGGTTCGCTTATTGGGGCCA | (SEQ ID NO: 409) |
| | AGGGACGCTGGTTACAGTGTCATCC | |
| | (SEQ ID NO: 408) | |
| hACI-8016- | GAAGTTCAACTGGTGCAGTCTGGAGCCG | GAAATTGTTCTGACTCAGAGCCCAGCTACC |
| 32B6C7- | AGGTCAAGAAACCCGGGGAGAGCCTAA | TTGTCCCTGTCCCCTGGAGAGAGGGCAAC |
| AB1_H2L2 | AGATTTCCTGCAAGATATCAGGTTATACT | TCTTTCGTGCCATGCTTCTCAGAATATAAA |
| | TTCACAGACCAGACAATTCATTGGGTGC | CGTCTGGTTGAGCTGGTACCAGAGAAAGC |
| | ACCAGATGCCGGGCAAGGGACTGGAAT | CCGGACAGGCCCCGAAACTGCTGATCTAT |
| | GGATGGGTTACATCTATCCTGGGGATGG | AAGTCTTCTAACCTCCACACAGGCATCCCT |
| | AAGCGCTATGTACAACGAGAAATTCCAG | GCGCGGTTCAGTGGTTCAGGCTCAGGGAC |
| | GGCCAAGTAACCATCAGTGCTGATAAGT | CGACTTCACGTTAACAATCAGCTCCCTCGA |
| | CTATCTCCACCGCATACCTCCAGTGGTCC | GCCCGAGGATTTTGCCGTGTACTATTGTCA |
| | AGCCTTAAAGCATCAGACACCGCCATGT | ACAAGGGCAGAGTTACCCACTAACCTTTG |
| | ATTACTGTGCCAGGCCACTGTATTCGTAC | GCCAGGGTACAAAGCTGGAAATTAAA |
| | GCTAGTTACTACTGGTTTGCGTATTGGG | (SEQ ID NO: 419) |
| | GCCAGGGCACTTTGGTCACGGTGTCTTC | |
| | C | |
| | (SEQ ID NO: 418) | |
| hACI-8016- | CAGGTGCAACTGGTCCAGAGCGGGGCT | GAAATCGTCCTCACGCAGTCACCTGCAACA |
| 32B6C7- | GAGGTGAAGAAGCCAGGGTCTAGTGTG | CTGTCTTTGAGCCCAGGCGAGAGAGCCAC |
| AB1_H3L3 | AAGGTCTCCTGCAAGATCTCCGGTTATTC | CCTGTCCTGCCATGCGTCTCAGAACATCAA |
| | CTTCACAGATCAGACGATTCACTGGATG | CGTGTGGCTGAGCTGGTATCAGCAAAAAC |
| | AGGCAGGCTCCCGGTCAAGGACTGGAAT | CGGGGCAAGCTCCACGGCTGCTAATCTAC |
| | GGATCGGCTACATATACCCGGGCGACGG | AAGTCCTCAAATCTTCACACAGGAATTCCT |
| | AAGTGCAATGTATAACGAAAAATTCCGG | GCCAGGTTTAGTGGCTCTGGGTCGGGCAC |
| | GGCCGCGCCACTTTGACTGCAGACAAAT | CGACTTCACTTTAACCATAAGTTCCCTGGA |
| | CAACCAGCACAGCCTACATGGAGCTCTCT | GCCCGAGGATTTCGCTGTTTATTACTGTCA |
| | TCTCTTCGATCAGAGGATACCGCTGTGTA | GCAGGGTCAGAGCTACCCCTTGACATTTG |
| | CTTTTGTGCGAGACCTCTGTATTCTTACG | GTCAGGGAACTAAACTCGAAATTAAG |
| | CCTCCTATTACTGGTTTGCCTATTGGGGC | (SEQ ID NO: 429) |
| | CAGGGAACCCTAGTTACAGTAAGCTCG | |
| | (SEQ ID NO: 428) | |
| hACI-8016- | CAGGTCCAACTCGTACAGAGTGGTGCCG | GAGATCGTCCTGACACAGTCCCCTGCCACA |
| 32B6C7- | AGGTTAAGAAGCCGGGGTCTTCCGTTAA | CTGTCACTGTCTCCAGGCGAGAGAGCTAC |
| AB1_H4L4 | AGTGTCCTGTAAGATCTCTGGCTACTCAT | CCTGTCTTGTCACGCCAGCCAGAACATCAA |
| | TCACAGATCAAACTATTCACTGGGTGCG | CGTGTACCTGTCCTGGTATCAGCAGAAGCC |
| | GCAGGCTCCCGGGCAGGGTCTTGAATGG | CGGCCAGGCTCCTAGACTGCTGATCTACAA |
| | ATGGGATATATCTACCCAGGAGACGGCA | GTCCTCCAACCTGCACACAGGCATCCCCGC |
| | GCGCTATGTATAACGAGAAGTTCCAGGG | CAGATTTTCCGGCTCTGGCTCTGGCACAGA |
| | ACGCGTGACCATAACAGCAGACAAATCC | CTTTACCCTGACCATCTCCAGCCTGGAACC |
| | ACTAGTACGGCCTATATGGAGCTGAGCT | TGAGGACTTCGCCGTGTACTACTGTCAGCA |
| | CACTAAGGAGCGAAGATACCGCCGTCTA | GGGCCAGTCTTACCCTCTGACCTTTGGCCA |
| | CTATTGCGCTAGACCTCTGTACTCGTACG | GGGCACCAAGCTGGAAATCAAG |
| | CATCTTATTACTGGTTTGCGTACTGGGGC | (SEQ ID NO: 439) |
| | CAGGGCACATTGGTGACCGTGTCCAGC | |
| | (SEQ ID NO: 438) | |
| hACI-8016- | CAGGTGCAGCTGGTTCAGTCTGGCGCCG | GAGATCGTCCTGACACAGTCCCCTGCCACA |
| 32B6C7- | AAGTGAAGAAACCTGGCTCCTCCGTGAA | CTGTCACTGTCTCCAGGCGAGAGAGCTAC |
| AB1_H5L4 | GGTGTCCTGCAAGATCTCCGGCTACACCT | CCTGTCTTGTCACGCCAGCCAGAACATCAA |
| | TTACCGACCAGACCATCCACTGGGTCCG | CGTGTACCTGTCCTGGTATCAGCAGAAGCC |
| | ACAGGCTCCAGGACAAGGCTTGGAGTG | CGGCCAGGCTCCTAGACTGCTGATCTACAA |
| | GATGGGCTACATCTATCCTGGCGACGGC | GTCCTCCAACCTGCACACAGGCATCCCCGC |
| | TCCGCCATGTACAACGAGAAGTTTCAGG | CAGATTTTCCGGCTCTGGCTCTGGCACAGA |
| | GCAGAGTGACCATCACCGCCGACAAGTC | CTTTACCCTGACCATCTCCAGCCTGGAACC |
| | TACCTCCACCGCCTACATGGAACTGTCCA | TGAGGACTTCGCCGTGTACTACTGTCAGCA |
| | GCCTGAGATCCGAGGACACCGCCGTGTA | GGGCCAGTCTTACCCTCTGACCTTTGGCCA |
| | CTACTGTGCCAGACCTCTGTACTCCTACG | GGGCACCAAGCTGGAAATCAAG |
| | CCTCCTACTATTGGTTCGCCTACTGGGGC | (SEQ ID NO: 439) |
| | CAGGGCACCCTGGTTACAGTTTCTTCT | |
| | (SEQ ID NO: 448) | |
| hACI-8016- | CAGGTGCAGCTGGTTCAGTCTGGCGCCG | GAGATCGTCCTGACACAGTCCCCTGCCACA |
| 32B6C7- | AAGTGAAGAAACCTGGCTCCTCCGTGAA | CTGTCACTGTCTCCAGGCGAGAGAGCTAC |
| AB1_H6L4 | GGTGTCCTGCAAGGCTTCTGGCTACACCT | CCTGTCTTGTCACGCCAGCCAGAACATCAA |
| | TTACCGACCAGACCATCCACTGGGTCCG | CGTGTACCTGTCCTGGTATCAGCAGAAGCC |
| | ACAGGCTCCAGGACAAGGCTTGGAGTG | CGGCCAGGCTCCTAGACTGCTGATCTACAA |
| | GATGGGCTACATCTATCCTGGCGACGGC | GTCCTCCAACCTGCACACAGGCATCCCCGC |
| | TCCGCCATGTACAACGAGAAGTTTCAGG | CAGATTTTCCGGCTCTGGCTCTGGCACAGA |
| | GCAGAGTGACCATCACCGCCGACAAGTC | CTTTACCCTGACCATCTCCAGCCTGGAACC |
| | TACCTCCACCGCCTACATGGAACTGTCCA | TGAGGACTTCGCCGTGTACTACTGTCAGCA |
| | GCCTGAGATCTGAGGACACCGCCGTGTA | GGGCCAGTCTTACCCTCTGACCTTTGGCCA |
| | CTACTGCGCCAGACCTCTGTACTCCTACG | GGGCACCAAGCTGGAAATCAAG |
| | CCTCCTACTATTGGTTCGCCTACTGGGGC | (SEQ ID NO: 439) |
| | CAGGGCACCCTGGTTACAGTTTCTTCT | |
| | (SEQ ID NO: 458) | |
| hACI-8016- | CAGGTGCAGCTGGTTCAGTCTGGCGCCG | GAGATCGTCCTGACACAGTCCCCTGCCACA |
| 32B6C7- | AAGTGAAGAAACCTGGCTCCTCCGTGAA | CTGTCACTGTCTCCAGGCGAGAGAGCTAC |
| AB1_H7L4 | GGTGTCCTGCAAGATCTCCGGCTACACCT | CCTGTCTTGTCACGCCAGCCAGAACATCAA |
| | TTACCGACCAGACCATCCACTGGGTCCG | CGTGTACCTGTCCTGGTATCAGCAGAAGCC |
| | ACAGGCTCCAGGACAAGGCTTGGAGTG | CGGCCAGGCTCCTAGACTGCTGATCTACAA |
| | GATGGGCTACATCTACCCTGGCGACTCCT | GTCCTCCAACCTGCACACAGGCATCCCCGC |
| | CCGCCATGTACAACGAGAAGTTCCAGGG | CAGATTTTCCGGCTCTGGCTCTGGCACAGA |
| | CAGAGTGACCATCACCGCCGACAAGTCT | CTTTACCCTGACCATCTCCAGCCTGGAACC |
| | ACCTCCACCGCCTACATGGAACTGTCCAG | TGAGGACTTCGCCGTGTACTACTGTCAGCA |
| | CCTGAGATCCGAGGACACCGCCGTGTAC | GGGCCAGTCTTACCCTCTGACCTTTGGCCA |
| | TACTGTGCCAGACCTCTGTACTCCTACGC | GGGCACCAAGCTGGAAATCAAG |
| | CTCCTACTATTGGTTCGCCTACTGGGGCC | (SEQ ID NO: 439) |
| | AGGGCACCCTGGTTACAGTTTCTTCT | |
| | (SEQ ID NO: 468) | |
| hACI-8016- | CAGGTGCAGCTGGTTCAGTCTGGCGCCG | GAGATCGTCCTGACACAGTCCCCTGCCACA |
| 32B6C7- | AAGTGAAGAAACCTGGCTCCTCCGTGAA | CTGTCACTGTCTCCAGGCGAGAGAGCTAC |
| AB1_H8 L4 | GGTGTCCTGCAAGATCTCCGGCTACACCT | CCTGTCTTGTCACGCCAGCCAGAACATCAA |
| | TTACCGACCAGACCATCCACTGGGTCCG | CGTGTACCTGTCCTGGTATCAGCAGAAGCC |
| | ACAGGCTCCAGGACAAGGCTTGGAGTG | CGGCCAGGCTCCTAGACTGCTGATCTACAA |
| | GATGGGCTACATCTACCCTGGCGACGCC | GTCCTCCAACCTGCACACAGGCATCCCCGC |
| | TCCGCCATGTACAACGAGAAGTTTCAGG | CAGATTTTCCGGCTCTGGCTCTGGCACAGA |
| | GCAGAGTGACCATCACCGCCGACAAGTC | CTTTACCCTGACCATCTCCAGCCTGGAACC |
| | TACCTCCACCGCCTACATGGAACTGTCCA | TGAGGACTTCGCCGTGTACTACTGTCAGCA |
| | GCCTGAGATCCGAGGACACCGCCGTGTA | GGGCCAGTCTTACCCTCTGACCTTTGGCCA |
| | CTACTGTGCCAGACCTCTGTACTCCTACG | GGGCACCAAGCTGGAAATCAAG |
| | CCTCCTACTATTGGTTCGCCTACTGGGGC | (SEQ ID NO: 439) |
| | CAGGGCACCCTGGTTACAGTTTCTTCT | |
| | (SEQ ID NO: 478) | |
| hACI-8016- | GAGGTGCAGCTGGTTCAGTCTGGCGCCG | GAGATCGTCCTGACACAGTCCCCTGCCACA |
| 32B6C7- | AAGTGAAGAAGCCTGGCGAGTCCCTGAA | CTGTCACTGTCTCCAGGCGAGAGAGCTAC |
| AB1_H9 L4 | GATCTCCTGCAAGATCAGCGGCTACTCCT | CCTGTCTTGTCACGCCAGCCAGAACATCAA |
| | TCACCGACCAGACCATCCATTGGGTGCA | CGTGTACCTGTCCTGGTATCAGCAGAAGCC |
| | CCAGATGCCTGGCAAAGGCCTGGAATGG | CGGCCAGGCTCCTAGACTGCTGATCTACAA |
| | ATGGGCTACATCTATCCCGGCGACGGCT | GTCCTCCAACCTGCACACAGGCATCCCCGC |
| | CCGCCATGTACAACGAGAAGTTTCAGGG | CAGATTTTCCGGCTCTGGCTCTGGCACAGA |
| | CCAAGTGACCATCTCCGCCGACAAGTCTA | CTTTACCCTGACCATCTCCAGCCTGGAACC |
| | TCTCCACCGCCTACCTGCAGTGGTCCTCT | TGAGGACTTCGCCGTGTACTACTGTCAGCA |
| | CTGAAGGCCTCTGACACCGCTATGTACTA | GGGCCAGTCTTACCCTCTGACCTTTGGCCA |
| | CTGCGCCAGACCTCTGTACTCCTACGCCT | GGGCACCAAGCTGGAAATCAAG |
| | CCTACTATTGGTTCGCCTACTGGGGCCAG | (SEQ ID NO: 439) |
| | GGCACCCTGGTTACAGTTTCTTCT | |
| | (SEQ ID NO: 488) | |
| hACI-8016- | GAGGTGCAGCTGGTTCAGTCTGGCGCCG | GAGATCGTCCTGACACAGTCCCCTGCCACA |
| 32B6C7- | AAGTGAAGAAGCCTGGCGAGTCCCTGAA | CTGTCACTGTCTCCAGGCGAGAGAGCTAC |
| AB1_H10 | GATCTCCTGCAAAGGCTCCGGCTACACCT | CCTGTCTTGTCACGCCAGCCAGAACATCAA |
| L4 | TCACCGACCAGACCATTCACTGGGTGCA | CGTGTACCTGTCCTGGTATCAGCAGAAGCC |
| | CCAGATGCCTGGCAAAGGCTTGGAGTGG | CGGCCAGGCTCCTAGACTGCTGATCTACAA |
| | ATGGGCTACATCTATCCCGGCGACGGCT | GTCCTCCAACCTGCACACAGGCATCCCCGC |
| | CCGCCATGTACAACGAGAAGTTTCAGGG | CAGATTTTCCGGCTCTGGCTCTGGCACAGA |
| | CCAAGTGACCATCTCCGCCGACAAGTCTA | CTTTACCCTGACCATCTCCAGCCTGGAACC |
| | TCTCCACCGCCTACCTGCAGTGGTCCTCT | TGAGGACTTCGCCGTGTACTACTGTCAGCA |
| | CTGAAGGCCTCTGACACCGCTATGTACTA | GGGCCAGTCTTACCCTCTGACCTTTGGCCA |
| | CTGCGCCAGACCTCTGTACTCCTACGCCT | GGGCACCAAGCTGGAAATCAAG |
| | CCTACTATTGGTTCGCCTACTGGGGCCAG | (SEQ ID NO: 439) |
| | GGCACCCTGGTTACAGTTTCTTCT | |
| | (SEQ ID NO: 498) | |
| hACI-8016- | GAGGTGCAGCTGGTTCAGTCTGGCGCCG | GAGATCGTCCTGACACAGTCCCCTGCCACA |
| 32B6C7- | AAGTGAAGAAGCCTGGCGAGTCCCTGAA | CTGTCACTGTCTCCAGGCGAGAGAGCTAC |
| AB1_H11 | GATCTCCTGCAAAGGCTCCGGCTACTCCT | CCTGTCTTGTCACGCCAGCCAGAACATCAA |
| L4 | TCACCGACCAGACCATTCACTGGGTGCA | CGTGTACCTGTCCTGGTATCAGCAGAAGCC |
| | CCAGATGCCTGGCAAAGGCTTGGAGTGG | CGGCCAGGCTCCTAGACTGCTGATCTACAA |
| | ATGGGCTACATCTATCCCGGCGACGGCT | GTCCTCCAACCTGCACACAGGCATCCCCGC |
| | CCGCCATGTACAACGAGAAGTTTCAGGG | CAGATTTTCCGGCTCTGGCTCTGGCACAGA |
| | CCAAGTGACCATCTCCGCCGACAAGTCTA | CTTTACCCTGACCATCTCCAGCCTGGAACC |
| | TCTCCACCGCCTACCTGCAGTGGTCCTCT | TGAGGACTTCGCCGTGTACTACTGTCAGCA |
| | CTGAAGGCCTCTGACACCGCTATGTACTA | GGGCCAGTCTTACCCTCTGACCTTTGGCCA |
| | CTGCGCCAGACCTCTGTACTCCTACGCCT | GGGCACCAAGCTGGAAATCAAG |
| | CCTACTATTGGTTCGCCTACTGGGGCCAG | (SEQ ID NO: 439) |
| | GGCACCCTGGTTACAGTTTCTTCT | |
| | (SEQ ID NO: 508) | |
| hACI-8016- | GAGGTGCAGCTGGTTCAGTCTGGCGCCG | GAGATCGTCCTGACACAGTCCCCTGCCACA |
| 32B6C7- | AAGTGAAGAAGCCTGGCGAGTCCCTGAA | CTGTCACTGTCTCCAGGCGAGAGAGCTAC |
| AB1_H12 | GATCTCCTGCAAGATCAGCGGCTACACCT | CCTGTCTTGTCACGCCAGCCAGAACATCAA |
| L4 | TCACCGACCAGACCATCCATTGGGTGCA | CGTGTACCTGTCCTGGTATCAGCAGAAGCC |
| | CCAGATGCCTGGCAAAGGCCTGGAATGG | CGGCCAGGCTCCTAGACTGCTGATCTACAA |
| | ATGGGCTACATCTACCCCGGCGACTCCTC | GTCCTCCAACCTGCACACAGGCATCCCCGC |
| | CGCCATGTACAACGAGAAGTTCCAGGGC | CAGATTTTCCGGCTCTGGCTCTGGCACAGA |
| | CAAGTGACCATCTCCGCCGACAAGTCTAT | CTTTACCCTGACCATCTCCAGCCTGGAACC |
| | CTCCACCGCCTACCTGCAGTGGTCCTCTC | TGAGGACTTCGCCGTGTACTACTGTCAGCA |
| | TGAAGGCCTCTGACACCGCTATGTACTAC | GGGCCAGTCTTACCCTCTGACCTTTGGCCA |
| | TGCGCCAGACCTCTGTACTCCTACGCCTC | GGGCACCAAGCTGGAAATCAAG |
| | CTACTATTGGTTCGCCTACTGGGGCCAG | (SEQ ID NO: 439) |
| | GGCACCCTGGTTACAGTTTCTTCT | |
| | (SEQ ID NO: 518) | |
| hACI-8016- | GAGGTGCAGCTGGTTCAGTCTGGCGCCG | GAGATCGTCCTGACACAGTCCCCTGCCACA |
| 32B6C7- | AAGTGAAGAAGCCTGGCGAGTCCCTGAA | CTGTCACTGTCTCCAGGCGAGAGAGCTAC |
| AB1_H13 | GATCTCCTGCAAGATCAGCGGCTACTCCT | CCTGTCTTGTCACGCCAGCCAGAACATCAA |
| L4 | TCACCGACCAGACCATCCATTGGGTGCA | CGTGTACCTGTCCTGGTATCAGCAGAAGCC |
| | CCAGATGCCTGGCAAAGGCCTGGAATGG | CGGCCAGGCTCCTAGACTGCTGATCTACAA |
| | ATGGGCTACATCTACCCCGGCGACTCCTC | GTCCTCCAACCTGCACACAGGCATCCCCGC |
| | CGCCATGTACAACGAGAAGTTCCAGGGC | CAGATTTTCCGGCTCTGGCTCTGGCACAGA |
| | CAAGTGACCATCTCCGCCGACAAGTCTAT | CTTTACCCTGACCATCTCCAGCCTGGAACC |
| | CTCCACCGCCTACCTGCAGTGGTCCTCTC | TGAGGACTTCGCCGTGTACTACTGTCAGCA |
| | TGAAGGCCTCTGACACCGCTATGTACTAC | GGGCCAGTCTTACCCTCTGACCTTTGGCCA |
| | TGCGCCAGACCTCTGTACTCCTACGCCTC | GGGCACCAAGCTGGAAATCAAG |
| | CTACTATTGGTTCGCCTACTGGGGCCAG | (SEQ ID NO: 439) |
| | GGCACCCTGGTTACAGTTTCTTCT | |
| | (SEQ ID NO: 528) | |
| hACI-8016- | CAGGTGCAGCTGGTTCAGTCTGGCGCCG | GAAATCGTCCTCACGCAGTCACCTGCAACA |
| 32B6C7- | AAGTGAAGAAACCTGGCTCCTCCGTGAA | CTGTCTTTGAGCCCAGGCGAGAGAGCCAC |
| AB1_H14L3 | GGTGTCCTGCAAGGCTTCTGGCTACACCT | CCTGTCCTGCCATGCGTCTCAGAACATCAA |
| | TTACCGACCAGACCATCCACTGGGTCCG | CGTGTGGCTGAGCTGGTATCAGCAAAAAC |
| | ACAGGCTCCAGGACAAGGCTTGGAGTG | CGGGGCAAGCTCCACGGCTGCTAATCTAC |
| | GATGGGCTACATCTATCCTGGCGACAGC | AAGTCCTCAAATCTTCACACAGGAATTCCT |
| | TCCGCCATGTACAACGAGAAGTTTCAGG | GCCAGGTTTAGTGGCTCTGGGTCGGGCAC |
| | GCAGAGTGACCATCACCGCCGACAAGTC | CGACTTCACTTTAACCATAAGTTCCCTGGA |
| | TACCTCCACCGCCTACATGGAACTGTCCA | GCCCGAGGATTTCGCTGTTTATTACTGTCA |
| | GCCTGAGATCTGAGGACACCGCCGTGTA | GCAGGGTCAGAGCTACCCCTTGACATTTG |
| | CTACTGCGCCAGACCTCTGTACTCCTACG | GTCAGGGAACTAAACTCGAAATTAAG |
| | CCTCCTACTATTGGTTCGCCTACTGGGGC | (SEQ ID NO: 429) |
| | CAGGGCACCCTGGTTACAGTTTCTTCT | |
| | (SEQ ID NO: 538) | |
| hACI-8016- | CAGGTGCAGCTGGTTCAGTCTGGCGCCG | GAAATCGTCCTCACGCAGTCACCTGCAACA |
| 32B6C7- | AAGTGAAGAAACCTGGCTCCTCCGTGAA | CTGTCTTTGAGCCCAGGCGAGAGAGCCAC |
| AB1_H15L3 | GGTGTCCTGCAAGGCTTCTGGCTACACCT | CCTGTCCTGCCATGCGTCTCAGAACATCAA |
| | TTACCGACCAGACCATCCACTGGGTCCG | CGTGTGGCTGAGCTGGTATCAGCAAAAAC |
| | ACAGGCTCCAGGACAAGGCTTGGAGTG | CGGGGCAAGCTCCACGGCTGCTAATCTAC |
| | GATGGGCTACATCTATCCTGGCGACGCC | AAGTCCTCAAATCTTCACACAGGAATTCCT |
| | TCCGCCATGTACAACGAGAAGTTTCAGG | GCCAGGTTTAGTGGCTCTGGGTCGGGCAC |
| | GCAGAGTGACCATCACCGCCGACAAGTC | CGACTTCACTTTAACCATAAGTTCCCTGGA |
| | TACCTCCACCGCCTACATGGAACTGTCCA | GCCCGAGGATTTCGCTGTTTATTACTGTCA |
| | GCCTGAGATCTGAGGACACCGCCGTGTA | GCAGGGTCAGAGCTACCCCTTGACATTTG |
| | CTACTGCGCCAGACCTCTGTACTCCTACG | GTCAGGGAACTAAACTCGAAATTAAG |
| | CCTCCTACTATTGGTTCGCCTACTGGGGC | (SEQ ID NO: 429) |
| | CAGGGCACCCTGGTTACAGTTTCTTCT | |
| | (SEQ ID NO: 548) | |
| hACI-8016- | GAAGTACAGCTCGTGCAGTCGGGTGCCG | GAAATTGTTCTGACTCAGAGCCCAGCTACC |
| 32B6C7- | AGGTCAAGAAGCCGGGCGAGAGCTTGA | TTGTCCCTGTCCCCTGGAGAGAGGGCAAC |
| AB1_H1L2 | AGATCTCATGTAAGATCAGCGGCTATAC | TCTTTCGTGCCATGCTTCTCAGAATATAAA |
| | CTTCACTGATCAGACCATACACTGGATGC | CGTCTGGTTGAGCTGGTACCAGAGAAAGC |
| | ATCAGATGCCTGGGAAAGGTCTAGAATG | CCGGACAGGCCCCGAAACTGCTGATCTAT |
| | GATCGGCTATATTTACCCAGGAGATGGA | AAGTCTTCTAACCTCCACACAGGCATCCCT |
| | TCCGCAATGTATAACGAGAAGTTTAGGG | GCGCGGTTCAGTGGTTCAGGCTCAGGGAC |
| | GACAGGCTACACTGAGTGCAGACAAATC | CGACTTCACGTTAACAATCAGCTCCCTCGA |
| | CATTAGCACTGCGTACCTGCAATGGAGTT | GCCCGAGGATTTTGCCGTGTACTATTGTCA |
| | CTCTCAAAGCCAGCGACACCGCTATGTAC | ACAAGGGCAGAGTTACCCACTAACCTTTG |
| | TTTTGCGCCAGACCCCTTTACTCTTATGCC | GCCAGGGTACAAAGCTGGAAATTAAA |
| | TCTTACTACTGGTTCGCTTATTGGGGCCA | (SEQ ID NO: 419) |
| | AGGGACGCTGGTTACAGTGTCATCC | |
| | (SEQ ID NO: 408) | |
| hACI-8016- | GAAGTACAGCTCGTGCAGTCGGGTGCCG | GAAATCGTCCTCACGCAGTCACCTGCAACA |
| 32B6C7- | AGGTCAAGAAGCCGGGCGAGAGCTTGA | CTGTCTTTGAGCCCAGGCGAGAGAGCCAC |
| AB1_H1L3 | AGATCTCATGTAAGATCAGCGGCTATAC | CCTGTCCTGCCATGCGTCTCAGAACATCAA |
| | CTTCACTGATCAGACCATACACTGGATGC | CGTGTGGCTGAGCTGGTATCAGCAAAAAC |
| | ATCAGATGCCTGGGAAAGGTCTAGAATG | CGGGGCAAGCTCCACGGCTGCTAATCTAC |
| | GATCGGCTATATTTACCCAGGAGATGGA | AAGTCCTCAAATCTTCACACAGGAATTCCT |
| | TCCGCAATGTATAACGAGAAGTTTAGGG | GCCAGGTTTAGTGGCTCTGGGTCGGGCAC |
| | GACAGGCTACACTGAGTGCAGACAAATC | CGACTTCACTTTAACCATAAGTTCCCTGGA |
| | CATTAGCACTGCGTACCTGCAATGGAGTT | GCCCGAGGATTTCGCTGTTTATTACTGTCA |
| | CTCTCAAAGCCAGCGACACCGCTATGTAC | GCAGGGTCAGAGCTACCCCTTGACATTTG |
| | TTTTGCGCCAGACCCCTTTACTCTTATGCC | GTCAGGGAACTAAACTCGAAATTAAG |
| | TCTTACTACTGGTTCGCTTATTGGGGCCA | (SEQ ID NO: 429) |
| | AGGGACGCTGGTTACAGTGTCATCC | |
| | (SEQ ID NO: 408) | |
| hACI-8016- | GAAGTTCAACTGGTGCAGTCTGGAGCCG | GATATTCAGATGACCCAATCCCCCTCTACC |
| 32B6C7- | AGGTCAAGAAACCCGGGGAGAGCCTAA | CTATCCGCTTCCGTGGGGGACCGGGTGAC |
| AB1_H2L1 | AGATTTCCTGCAAGATATCAGGTTATACT | AATCACGTGCCATGCAAGCCAGAATATCA |
| | TTCACAGACCAGACAATTCATTGGGTGC | ACGTTTGGCTGAGCTGGTACCAGAGAAAG |
| | ACCAGATGCCGGGCAAGGGACTGGAAT | CCAGGAAAGGCCCCTAAACTTTTGATTTAC |
| | GGATGGGTTACATCTATCCTGGGGATGG | AAGTCATCCAACTTACACACAGGAGTCCCC |
| | AAGCGCTATGTACAACGAGAAATTCCAG | TCGAGGTTTAGCGGCTCAGGTAGTGGCAC |
| | GGCCAAGTAACCATCAGTGCTGATAAGT | AGAATTCACTCTGACTATCTCTAGTCTGCA |
| | CTATCTCCACCGCATACCTCCAGTGGTCC | GCCAGATGACTTCGCCACCTACTATTGTCA |
| | AGCCTTAAAGCATCAGACACCGCCATGT | ACAGGGGCAGTCTTATCCTCTCACCTTTGG |
| | ATTACTGTGCCAGGCCACTGTATTCGTAC | CCAGGGTACTAAACTCGAGATAAAG |
| | GCTAGTTACTACTGGTTTGCGTATTGGG | (SEQ ID NO: 409) |
| | GCCAGGGCACTTTGGTCACGGTGTCTTC | |
| | C | |
| | (SEQ ID NO: 418) | |
| hACI-8016- | GAAGTTCAACTGGTGCAGTCTGGAGCCG | GAAATCGTCCTCACGCAGTCACCTGCAACA |
| 32B6C7- | AGGTCAAGAAACCCGGGGAGAGCCTAA | CTGTCTTTGAGCCCAGGCGAGAGAGCCAC |
| AB1_H2L3 | AGATTTCCTGCAAGATATCAGGTTATACT | CCTGTCCTGCCATGCGTCTCAGAACATCAA |
| | TTCACAGACCAGACAATTCATTGGGTGC | CGTGTGGCTGAGCTGGTATCAGCAAAAAC |
| | ACCAGATGCCGGGCAAGGGACTGGAAT | CGGGGCAAGCTCCACGGCTGCTAATCTAC |
| | GGATGGGTTACATCTATCCTGGGGATGG | AAGTCCTCAAATCTTCACACAGGAATTCCT |
| | AAGCGCTATGTACAACGAGAAATTCCAG | GCCAGGTTTAGTGGCTCTGGGTCGGGCAC |
| | GGCCAAGTAACCATCAGTGCTGATAAGT | CGACTTCACTTTAACCATAAGTTCCCTGGA |
| | CTATCTCCACCGCATACCTCCAGTGGTCC | GCCCGAGGATTTCGCTGTTTATTACTGTCA |
| | AGCCTTAAAGCATCAGACACCGCCATGT | GCAGGGTCAGAGCTACCCCTTGACATTTG |
| | ATTACTGTGCCAGGCCACTGTATTCGTAC | GTCAGGGAACTAAACTCGAAATTAAG |
| | GCTAGTTACTACTGGTTTGCGTATTGGG | (SEQ ID NO: 429) |
| | GCCAGGGCACTTTGGTCACGGTGTCTTC | |
| | C | |
| | (SEQ ID NO: 418) | |
| hACI-8016- | GAAGTTCAACTGGTGCAGTCTGGAGCCG | GAGATCGTCCTGACACAGTCCCCTGCCACA |
| 32B6C7- | AGGTCAAGAAACCCGGGGAGAGCCTAA | CTGTCACTGTCTCCAGGCGAGAGAGCTAC |
| AB1_H2L4 | AGATTTCCTGCAAGATATCAGGTTATACT | CCTGTCTTGTCACGCCAGCCAGAACATCAA |
| | TTCACAGACCAGACAATTCATTGGGTGC | CGTGTACCTGTCCTGGTATCAGCAGAAGCC |
| | ACCAGATGCCGGGCAAGGGACTGGAAT | CGGCCAGGCTCCTAGACTGCTGATCTACAA |
| | GGATGGGTTACATCTATCCTGGGGATGG | GTCCTCCAACCTGCACACAGGCATCCCCGC |
| | AAGCGCTATGTACAACGAGAAATTCCAG | CAGATTTTCCGGCTCTGGCTCTGGCACAGA |
| | GGCCAAGTAACCATCAGTGCTGATAAGT | CTTTACCCTGACCATCTCCAGCCTGGAACC |
| | CTATCTCCACCGCATACCTCCAGTGGTCC | TGAGGACTTCGCCGTGTACTACTGTCAGCA |
| | AGCCTTAAAGCATCAGACACCGCCATGT | GGGCCAGTCTTACCCTCTGACCTTTGGCCA |
| | ATTACTGTGCCAGGCCACTGTATTCGTAC | GGGCACCAAGCTGGAAATCAAG |
| | GCTAGTTACTACTGGTTTGCGTATTGGG | (SEQ ID NO: 439) |
| | GCCAGGGCACTTTGGTCACGGTGTCTTC | |
| | C | |
| | (SEQ ID NO: 418) | |
| hACI-8016- | CAGGTGCAACTGGTCCAGAGCGGGGCT | GATATTCAGATGACCCAATCCCCCTCTACC |
| 32B6C7- | GAGGTGAAGAAGCCAGGGTCTAGTGTG | CTATCCGCTTCCGTGGGGGACCGGGTGAC |
| AB1_H3L1 | AAGGTCTCCTGCAAGATCTCCGGTTATTC | AATCACGTGCCATGCAAGCCAGAATATCA |
| | CTTCACAGATCAGACGATTCACTGGATG | ACGTTTGGCTGAGCTGGTACCAGAGAAAG |
| | AGGCAGGCTCCCGGTCAAGGACTGGAAT | CCAGGAAAGGCCCCTAAACTTTTGATTTAC |
| | GGATCGGCTACATATACCCGGGCGACGG | AAGTCATCCAACTTACACACAGGAGTCCCC |
| | AAGTGCAATGTATAACGAAAAATTCCGG | TCGAGGTTTAGCGGCTCAGGTAGTGGCAC |
| | GGCCGCGCCACTTTGACTGCAGACAAAT | AGAATTCACTCTGACTATCTCTAGTCTGCA |
| | CAACCAGCACAGCCTACATGGAGCTCTCT | GCCAGATGACTTCGCCACCTACTATTGTCA |
| | TCTCTTCGATCAGAGGATACCGCTGTGTA | ACAGGGGCAGTCTTATCCTCTCACCTTTGG |
| | CTTTTGTGCGAGACCTCTGTATTCTTACG | CCAGGGTACTAAACTCGAGATAAAG |
| | CCTCCTATTACTGGTTTGCCTATTGGGGC | (SEQ ID NO: 409) |
| | CAGGGAACCCTAGTTACAGTAAGCTCG | |
| | (SEQ ID NO: 428) | |
| hACI-8016- | CAGGTGCAACTGGTCCAGAGCGGGGCT | GAAATTGTTCTGACTCAGAGCCCAGCTACC |
| 32B6C7- | GAGGTGAAGAAGCCAGGGTCTAGTGTG | TTGTCCCTGTCCCCTGGAGAGAGGGCAAC |
| AB1_H3L2 | AAGGTCTCCTGCAAGATCTCCGGTTATTC | TCTTTCGTGCCATGCTTCTCAGAATATAAA |
| | CTTCACAGATCAGACGATTCACTGGATG | CGTCTGGTTGAGCTGGTACCAGAGAAAGC |
| | AGGCAGGCTCCCGGTCAAGGACTGGAAT | CCGGACAGGCCCCGAAACTGCTGATCTAT |
| | GGATCGGCTACATATACCCGGGCGACGG | AAGTCTTCTAACCTCCACACAGGCATCCCT |
| | AAGTGCAATGTATAACGAAAAATTCCGG | GCGCGGTTCAGTGGTTCAGGCTCAGGGAC |
| | GGCCGCGCCACTTTGACTGCAGACAAAT | CGACTTCACGTTAACAATCAGCTCCCTCGA |
| | CAACCAGCACAGCCTACATGGAGCTCTCT | GCCCGAGGATTTTGCCGTGTACTATTGTCA |
| | TCTCTTCGATCAGAGGATACCGCTGTGTA | ACAAGGGCAGAGTTACCCACTAACCTTTG |
| | CTTTTGTGCGAGACCTCTGTATTCTTACG | GCCAGGGTACAAAGCTGGAAATTAAA |
| | CCTCCTATTACTGGTTTGCCTATTGGGGC | (SEQ ID NO: 419) |
| | CAGGGAACCCTAGTTACAGTAAGCTCG | |
| | (SEQ ID NO: 428) | |
| hACI-8016- | CAGGTCCAACTCGTACAGAGTGGTGCCG | GATATTCAGATGACCCAATCCCCCTCTACC |
| 32B6C7- | AGGTTAAGAAGCCGGGGTCTTCCGTTAA | CTATCCGCTTCCGTGGGGGACCGGGTGAC |
| AB1_H4L1 | AGTGTCCTGTAAGATCTCTGGCTACTCAT | AATCACGTGCCATGCAAGCCAGAATATCA |
| | TCACAGATCAAACTATTCACTGGGTGCG | ACGTTTGGCTGAGCTGGTACCAGAGAAAG |
| | GCAGGCTCCCGGGCAGGGTCTTGAATGG | CCAGGAAAGGCCCCTAAACTTTTGATTTAC |
| | ATGGGATATATCTACCCAGGAGACGGCA | AAGTCATCCAACTTACACACAGGAGTCCCC |
| | GCGCTATGTATAACGAGAAGTTCCAGGG | TCGAGGTTTAGCGGCTCAGGTAGTGGCAC |
| | ACGCGTGACCATAACAGCAGACAAATCC | AGAATTCACTCTGACTATCTCTAGTCTGCA |
| | ACTAGTACGGCCTATATGGAGCTGAGCT | GCCAGATGACTTCGCCACCTACTATTGTCA |
| | CACTAAGGAGCGAAGATACCGCCGTCTA | ACAGGGGCAGTCTTATCCTCTCACCTTTGG |
| | CTATTGCGCTAGACCTCTGTACTCGTACG | CCAGGGTACTAAACTCGAGATAAAG |
| | CATCTTATTACTGGTTTGCGTACTGGGGC | (SEQ ID NO: 409) |
| | CAGGGCACATTGGTGACCGTGTCCAGC | |
| | (SEQ ID NO: 438) | |
| hACI-8016- | CAGGTCCAACTCGTACAGAGTGGTGCCG | GAAATTGTTCTGACTCAGAGCCCAGCTACC |
| 32B6C7- | AGGTTAAGAAGCCGGGGTCTTCCGTTAA | TTGTCCCTGTCCCCTGGAGAGAGGGCAAC |
| AB1_H4L2 | AGTGTCCTGTAAGATCTCTGGCTACTCAT | TCTTTCGTGCCATGCTTCTCAGAATATAAA |
| | TCACAGATCAAACTATTCACTGGGTGCG | CGTCTGGTTGAGCTGGTACCAGAGAAAGC |
| | GCAGGCTCCCGGGCAGGGTCTTGAATGG | CCGGACAGGCCCCGAAACTGCTGATCTAT |
| | ATGGGATATATCTACCCAGGAGACGGCA | AAGTCTTCTAACCTCCACACAGGCATCCCT |
| | GCGCTATGTATAACGAGAAGTTCCAGGG | GCGCGGTTCAGTGGTTCAGGCTCAGGGAC |
| | ACGCGTGACCATAACAGCAGACAAATCC | CGACTTCACGTTAACAATCAGCTCCCTCGA |
| | ACTAGTACGGCCTATATGGAGCTGAGCT | GCCCGAGGATTTTGCCGTGTACTATTGTCA |
| | CACTAAGGAGCGAAGATACCGCCGTCTA | ACAAGGGCAGAGTTACCCACTAACCTTTG |
| | CTATTGCGCTAGACCTCTGTACTCGTACG | GCCAGGGTACAAAGCTGGAAATTAAA |
| | CATCTTATTACTGGTTTGCGTACTGGGGC | (SEQ ID NO: 419) |
| | CAGGGCACATTGGTGACCGTGTCCAGC | |
| | (SEQ ID NO: 438) | |
| hACI-8016- | CAGGTCCAACTCGTACAGAGTGGTGCCG | GAAATCGTCCTCACGCAGTCACCTGCAACA |
| 32B6C7- | AGGTTAAGAAGCCGGGGTCTTCCGTTAA | CTGTCTTTGAGCCCAGGCGAGAGAGCCAC |
| AB1_H4L3 | AGTGTCCTGTAAGATCTCTGGCTACTCAT | CCTGTCCTGCCATGCGTCTCAGAACATCAA |
| | TCACAGATCAAACTATTCACTGGGTGCG | CGTGTGGCTGAGCTGGTATCAGCAAAAAC |
| | GCAGGCTCCCGGGCAGGGTCTTGAATGG | CGGGGCAAGCTCCACGGCTGCTAATCTAC |
| | ATGGGATATATCTACCCAGGAGACGGCA | AAGTCCTCAAATCTTCACACAGGAATTCCT |
| | GCGCTATGTATAACGAGAAGTTCCAGGG | GCCAGGTTTAGTGGCTCTGGGTCGGGCAC |
| | ACGCGTGACCATAACAGCAGACAAATCC | CGACTTCACTTTAACCATAAGTTCCCTGGA |
| | ACTAGTACGGCCTATATGGAGCTGAGCT | GCCCGAGGATTTCGCTGTTTATTACTGTCA |
| | CACTAAGGAGCGAAGATACCGCCGTCTA | GCAGGGTCAGAGCTACCCCTTGACATTTG |
| | CTATTGCGCTAGACCTCTGTACTCGTACG | GTCAGGGAACTAAACTCGAAATTAAG |
| | CATCTTATTACTGGTTTGCGTACTGGGGC | (SEQ ID NO: 429) |
| | CAGGGCACATTGGTGACCGTGTCCAGC | |
| | (SEQ ID NO: 438) | |
| hACI-8016- | CAGGTGCAGCTGGTTCAGTCTGGCGCCG | GAAATCGTCCTCACGCAGTCACCTGCAACA |
| 32B6C7- | AAGTGAAGAAACCTGGCTCCTCCGTGAA | CTGTCTTTGAGCCCAGGCGAGAGAGCCAC |
| AB1_H6L3 | GGTGTCCTGCAAGGCTTCTGGCTACACCT | CCTGTCCTGCCATGCGTCTCAGAACATCAA |
| | TTACCGACCAGACCATCCACTGGGTCCG | CGTGTGGCTGAGCTGGTATCAGCAAAAAC |
| | ACAGGCTCCAGGACAAGGCTTGGAGTG | CGGGGCAAGCTCCACGGCTGCTAATCTAC |
| | GATGGGCTACATCTATCCTGGCGACGGC | AAGTCCTCAAATCTTCACACAGGAATTCCT |
| | TCCGCCATGTACAACGAGAAGTTTCAGG | GCCAGGTTTAGTGGCTCTGGGTCGGGCAC |
| | GCAGAGTGACCATCACCGCCGACAAGTC | CGACTTCACTTTAACCATAAGTTCCCTGGA |
| | TACCTCCACCGCCTACATGGAACTGTCCA | GCCCGAGGATTTCGCTGTTTATTACTGTCA |
| | GCCTGAGATCTGAGGACACCGCCGTGTA | GCAGGGTCAGAGCTACCCCTTGACATTTG |
| | CTACTGCGCCAGACCTCTGTACTCCTACG | GTCAGGGAACTAAACTCGAAATTAAG |
| | CCTCCTACTATTGGTTCGCCTACTGGGGC | (SEQ ID NO: 429) |
| | CAGGGCACCCTGGTTACAGTTTCTTCT | |
| | (SEQ ID NO: 458) | |
| hACI-8016- | GAGGTGCAGCTGGTTCAGTCTGGCGCCG | GAAATCGTCCTCACGCAGTCACCTGCAACA |
| 32B6C7- | AAGTGAAGAAGCCTGGCGAGTCCCTGAA | CTGTCTTTGAGCCCAGGCGAGAGAGCCAC |
| AB1_H9 L3 | GATCTCCTGCAAGATCAGCGGCTACTCCT | CCTGTCCTGCCATGCGTCTCAGAACATCAA |
| | TCACCGACCAGACCATCCATTGGGTGCA | CGTGTGGCTGAGCTGGTATCAGCAAAAAC |
| | CCAGATGCCTGGCAAAGGCCTGGAATGG | CGGGGCAAGCTCCACGGCTGCTAATCTAC |
| | ATGGGCTACATCTATCCCGGCGACGGCT | AAGTCCTCAAATCTTCACACAGGAATTCCT |
| | CCGCCATGTACAACGAGAAGTTTCAGGG | GCCAGGTTTAGTGGCTCTGGGTCGGGCAC |
| | CCAAGTGACCATCTCCGCCGACAAGTCTA | CGACTTCACTTTAACCATAAGTTCCCTGGA |
| | TCTCCACCGCCTACCTGCAGTGGTCCTCT | GCCCGAGGATTTCGCTGTTTATTACTGTCA |
| | CTGAAGGCCTCTGACACCGCTATGTACTA | GCAGGGTCAGAGCTACCCCTTGACATTTG |
| | CTGCGCCAGACCTCTGTACTCCTACGCCT | GTCAGGGAACTAAACTCGAAATTAAG |
| | CCTACTATTGGTTCGCCTACTGGGGCCAG | (SEQ ID NO: 429) |
| | GGCACCCTGGTTACAGTTTCTTCT | |
| | (SEQ ID NO: 488) | |
| hACI-8016- | GAGGTGCAGCTGGTTCAGTCTGGCGCCG | GAAATCGTCCTCACGCAGTCACCTGCAACA |
| 32B6C7- | AAGTGAAGAAGCCTGGCGAGTCCCTGAA | CTGTCTTTGAGCCCAGGCGAGAGAGCCAC |
| AB1_H13 | GATCTCCTGCAAGATCAGCGGCTACTCCT | CCTGTCCTGCCATGCGTCTCAGAACATCAA |
| L3 | TCACCGACCAGACCATCCATTGGGTGCA | CGTGTGGCTGAGCTGGTATCAGCAAAAAC |
| | CCAGATGCCTGGCAAAGGCCTGGAATGG | CGGGGCAAGCTCCACGGCTGCTAATCTAC |
| | ATGGGCTACATCTACCCCGGCGACTCCTC | AAGTCCTCAAATCTTCACACAGGAATTCCT |
| | CGCCATGTACAACGAGAAGTTCCAGGGC | GCCAGGTTTAGTGGCTCTGGGTCGGGCAC |
| | CAAGTGACCATCTCCGCCGACAAGTCTAT | CGACTTCACTTTAACCATAAGTTCCCTGGA |
| | CTCCACCGCCTACCTGCAGTGGTCCTCTC | GCCCGAGGATTTCGCTGTTTATTACTGTCA |
| | TGAAGGCCTCTGACACCGCTATGTACTAC | GCAGGGTCAGAGCTACCCCTTGACATTTG |
| | TGCGCCAGACCTCTGTACTCCTACGCCTC | GTCAGGGAACTAAACTCGAAATTAAG |
| | CTACTATTGGTTCGCCTACTGGGGCCAG | (SEQ ID NO: 429) |
| | GGCACCCTGGTTACAGTTTCTTCT | |
| | (SEQ ID NO: 528) | |

TABLE 20
Amino acid sequences of the humanized Heavy Chain and humanized Light Chain Variable Domains (VH and VL) and their CDRs

| Antibody | | | | | | | | |
|---|---|---|---|---|---|---|---|---|
| name | VH sequence | VH-CDRH1 | VH-CDRH2 | VH-CDRH3 | VL sequence | VL-CDRH1 | VL-CDRH2 | VL-CDRH3 |
| ACI- | QVQLQQSDAELVKPGASV | DQTIH | YIYPGDGS | PLYSYASYY | DIQMNQSPSSLSASLGDTITI | HASQNINV | KSSNLHT | QQGQSYPL |
| 8016- | KISCKISGYTFTDQTIHWM | (SEQ ID | AMYNEKF | WFAY | TCHASQNINVWLSWYQRKP | WLS | (SEQ ID | T |
| 32B6C7- | NQRPEQGLEWIGYIYPGD | NO: 201) | RG | (SEQ ID | GNIPKLLIYKSSNLHTGVPSRF | (SEQ ID | NO: 206) | (SEQ ID NO: |
| AB1 | GSAMYNEKFRGKATLTAD | | (SEQ ID | NO: 203) | SGSGSGTGFTLTISSLOPEDIA | NO: 205) | | 207) |
| | KSSSTAYMQLNSLTSEDSA | | NO: 202) | | TYYCQQGQSYPLTFGGGTKL | | | |
| | VYFCARPLYSYASYYWFAY | | | | EIK | | | |
| | WGQGTLVTVSA | | | | (SEQ ID NO: 204) | | | |
| | (SEQ ID NO: 200) | | | | | | | |
| hACI- | EVQLVQSGAEVKKPGESLK | DQTIH | YIYPGDGS | PLYSYASYY | DIQMTQSPSTLSASVGDRVT | HASQNINV | KSSNLHT | QQGQSYPL |
| 8016- | ISCKISGYTFTDQTIHWMH | (SEQ ID | AMYNEKF | WFAY | ITCHASQNINVWLSWYQRK | WLS | (SEQ ID | T |
| 32B6C7- | QMPGKGLEWIGYIYPGDG | NO: 201) | RG | (SEQ ID | PGKAPKLLIYKSSNLHTGVPS | (SEQ ID | NO: 206) | (SEQ ID NO: |
| AB1 | SAMYNEKFRGQATLSADK | | (SEQ ID | NO: 203) | RFSGSGSGTEFTLTISSLQPD | NO: 205) | | 207) |
| H1L1 | SISTAYLQWSSLKASDTAM | | NO: 202) | | DFATYYCQQGQSYPLTFGQ | | | |
| | YFCARPLYSYASYYWFAYW | | | | GTKLEIK | | | |
| | GQGTLVTVSS | | | | (SEQ ID NO: 404) | | | |
| | (SEQ ID NO: 400) | | | | | | | |
| hACI- | EVQLVQSGAEVKKPGESLK | DQTIH | YIYPGDGS | PLYSYASYY | EIVLTQSPATLSLSPGERATLS | HASQNINV | KSSNLHT | QQGQSYPL |
| 8016- | ISCKISGYTFTDQTIHWVH | (SEQ ID | AMYNEKF | WFAY | CHASQNINVWLSWYQRKP | WLS | (SEQ ID | T |
| 32B6C7- | QMPGKGLEWMGYIYPGD | NO: 201) | QG | (SEQ ID | GQAPKLLIYKSSNLHTGIPAR | (SEQ ID | NO: 206) | (SEQ ID NO: |
| AB1 | GSAMYNEKFQGQVTISAD | | (SEQ ID | NO: 203) | FSGSGSGTDFTLTISSLEPEDF | NO: 205) | | 207) |
| H2L2 | KSISTAYLQWSSLKASDTA | | NO: 412) | | AVYYCQQGQSYPLTFGQGT | | | |
| | MYYCARPLYSYASYYWFAY | | | | KLEIK | | | |
| | WGQGTLVTVSS | | | | (SEQ ID NO: 414) | | | |
| | (SEQ ID NO: 410) | | | | | | | |
| hACI- | QVQLVQSGAEVKKPGSSV | DQTIH | YIYPGDGS | PLYSYASYY | EIVLTQSPATLSLSPGERATLS | HASQNINV | KSSNLHT | QQGQSYPL |
| 8016- | KVSCKISGYSFTDQTIHWM | (SEQ ID | AMYNEKF | WFAY | CHASQNINVWLSWYQQKP | WLS | (SEQ ID | T |
| 32B6C7- | RQAPGQGLEWIGYIYPGD | NO: 201) | RG | (SEQ ID | GQAPRLLIYKSSNLHTGIPAR | (SEQ ID | NO: 206) | (SEQ ID NO: |
| AB1 | GSAMYNEKFRGRATLTAD | | (SEQ ID | NO: 203) | FSGSGSGTDFTLTISSLEPEDF | NO: 205) | | 207) |
| H3L3 | KSTSTAYMELSSLRSEDTAV | | NO: 202) | | AVYYCQQGQSYPLTFGQGT | | | |
| | YFCARPLYSYASYYWFAYW | | | | KLEIK | | | |
| | GQGTLVTVSS | | | | (SEQ ID NO: 424) | | | |
| | (SEQ ID NO: 420) | | | | | | | |
| hACI- | QVQLVQSGAEVKKPGSSV | DQTIH | YIYPGDGS | PLYSYASYY | EIVLTQSPATLSLSPGERATLS | HASQNINV | KSSNLHT | QQGQSYPL |
| 8016- | KVSCKISGYSFTDQTIHWV | (SEQ ID | AMYNEKF | WFAY | CHASQNINVYLSWYQQKPG | YLS | (SEQ ID | T |
| 32B6C7- | RQAPGQGLEWMGYIYPG | NO: 201) | QG | (SEQ ID | QAPRLLIYKSSNLHTGIPARFS | (SEQ ID | NO: 206) | (SEQ ID NO: |
| AB1 | DGSAMYNEKFQGRVTITA | | (SEQ ID | NO: 203) | GSGSGTDFTLTISSLEPEDFA | NO: 435) | | 207) |
| H4L4 | DKSTSTAYMELSSLRSEDT | | NO: 412) | | VYYCQQGQSYPLTFGQGTKL | | | |
| | AVYYCARPLYSYASYYWFA | | | | EIK | | | |
| | YWGQGTLVTVSS | | | | (SEQ ID NO: 434) | | | |
| | (SEQ ID NO: 430) | | | | | | | |
| hACI- | QVQLVQSGAEVKKPGSSV | DQTIH | YIYPGDGS | PLYSYASYY | EIVLTQSPATLSLSPGERATLS | HASQNINV | KSSNLHT | QQGQSYPL |
| 8016- | KVSCKISGYTFTDQTIHWV | (SEQ ID | AMYNEKF | WFAY | CHASQNINVYLSWYQQKPG | YLS | (SEQ ID | T |
| 32B6C7- | RQAPGQGLEWMGYIYPG | NO: 201) | QG | (SEQ ID | QAPRLLIYKSSNLHTGIPARFS | (SEQ ID | NO: 206) | (SEQ ID NO: |
| AB1 | DGSAMYNEKFQGRVTITA | | (SEQ ID | NO: 203) | GSGSGTDFTLTISSLEPEDFA | NO: 435) | | 207) |
| H5L4 | DKSTSTAYMELSSLRSEDT | | NO: 412) | | VYYCQQGQSYPLTFGQGTKL | | | |
| | AVYYCARPLYSYASYYWFA | | | | EIK | | | |
| | YWGQGTLVTVSS | | | | (SEQ ID NO: 434) | | | |
| | (SEQ ID NO: 440) | | | | | | | |
| hACI- | QVQLVQSGAEVKKPGSSV | DQTIH | YIYPGDGS | PLYSYASYY | EIVLTQSPATLSLSPGERATLS | HASQNINV | KSSNLHT | QQGQSYPL |
| 8016- | KVSCKASGYTFTDQTIHWV | (SEQ ID | AMYNEKF | WFAY | CHASQNINVYLSWYQQKPG | YLS | (SEQ ID | T |
| 32B6C7- | RQAPGQGLEWMGYIYPG | NO: 201) | QG | (SEQ ID | QAPRLLIYKSSNLHTGIPARFS | (SEQ ID | NO: 206) | (SEQ ID NO: |
| AB1 | DGSAMYNEKFQGRVTITA | | (SEQ ID | NO: 203) | GSGSGTDFTLTISSLEPEDFA | NO: 435) | | 207) |
| H6L4 | DKSTSTAYMELSSLRSEDT | | NO: 412) | | VYYCQQGQSYPLTFGQGTKL | | | |
| | AVYYCARPLYSYASYYWFA | | | | EIK | | | |
| | YWGQGTLVTVSS | | | | (SEQ ID NO: 434) | | | |
| | (SEQ ID NO: 450) | | | | | | | |
| hACI- | QVQLVQSGAEVKKPGSSV | DQTIH | YIYPGDSSA | PLYSYASYY | EIVLTQSPATLSLSPGERATLS | HASQNINV | KSSNLHT | QQGQSYPL |
| 8016- | KVSCKISGYTFTDQTIHWV | (SEQ ID | MYNEKFQ | WFAY | CHASQNINVYLSWYQQKPG | YLS | (SEQ ID | T |
| 32B6C7- | RQAPGQGLEWMGYIYPG | NO: 201) | G | (SEQ ID | QAPRLLIYKSSNLHTGIPARFS | (SEQ ID | NO: 206) | (SEQ ID NO: |
| AB1 | DSSAMYNEKFQGRVTITA | | (SEQ ID | NO: 203) | GSGSGTDFTLTISSLEPEDFA | NO: 435) | | 207) |
| H7L4 | DKSTSTAYMELSSLRSEDT | | NO: 462) | | VYYCQQGQSYPLTFGQGTKL | | | |
| | AVYYCARPLYSYASYYWFA | | | | EIK | | | |
| | YWGQGTLVTVSS | | | | (SEQ ID NO: 434) | | | |
| | (SEQ ID NO: 460) | | | | | | | |
| hACI- | QVQLVQSGAEVKKPGSSV | DQTIH | YIYPGDAS | PLYSYASYY | EIVLTQSPATLSLSPGERATLS | HASQNINV | KSSNLHT | QQGQSYPL |
| 8016- | KVSCKISGYTFTDQTIHWV | (SEQ ID | AMYNEKF | WFAY | CHASQNINVYLSWYQQKPG | YLS | (SEQ ID | T |
| 32B6C7- | RQAPGQGLEWMGYIYPG | NO: 201) | QG | (SEQ ID | QAPRLLIYKSSNLHTGIPARFS | (SEQ ID | NO: 206) | (SEQ ID NO: |
| AB1 | DASAMYNEKFQGRVTITA | | (SEQ ID | NO: 203) | GSGSGTDFTLTISSLEPEDFA | NO: 435) | | 207) |
| H8 L4 | DKSTSTAYMELSSLRSEDT | | NO: 472) | | VYYCQQGQSYPLTFGQGTKL | | | |
| | AVYYCARPLYSYASYYWFA | | | | EIK | | | |
| | YWGQGTLVTVSS | | | | (SEQ ID NO: 434) | | | |
| | (SEQ ID NO: 470) | | | | | | | |
| hACI- | EVQLVQSGAEVKKPGESLK | DQTIH | YIYPGDGS | PLYSYASYY | EIVLTQSPATLSLSPGERATLS | HASQNINV | KSSNLHT | QQGQSYPL |
| 8016- | ISCKISGYSFTDQTIHWVH | (SEQ ID | AMYNEKF | WFAY | CHASQNINVYLSWYQQKPG | YLS | (SEQ ID | T |
| 32B6C7- | QMPGKGLEWMGYIYPGD | NO: 201) | QG | (SEQ ID | QAPRLLIYKSSNLHTGIPARFS | (SEQ ID | NO: 206) | (SEQ ID NO: |
| AB1 | GSAMYNEKFQGQVTISAD | | (SEQ ID | NO: 203) | GSGSGTDFTLTISSLEPEDFA | NO: 435) | | 207) |
| H9 L4 | KSISTAYLOWSSLKASDTA | | NO:412) | | VYYCQQGQSYPLTFGQGTKL | | | |
| | MYYCARPLYSYASYYWFAY | | | | EIK | | | |
| | WGQGTLVTVSS | | | | (SEQ ID NO: 434) | | | |
| | (SEQ ID NO: 480) | | | | | | | |
| hACI- | EVQLVQSGAEVKKPGESLK | DQTIH | YIYPGDGS | PLYSYASYY | EIVLTQSPATLSLSPGERATLS | HASQNINV | KSSNLHT | QQGQSYPL |
| 8016- | ISCKGSGYTFTDQTIHWVH | (SEQ ID | AMYNEKF | WFAY | CHASQNINVYLSWYQQKPG | YLS | (SEQ ID | T |
| 32B6C7- | QMPGKGLEWMGYIYPGD | NO: 201) | QG | (SEQ ID | QAPRLLIYKSSNLHTGIPARFS | (SEQ ID | NO: 206) | (SEQ ID NO: |
| AB1 | GSAMYNEKFQGQVTISAD | | (SEQ ID | NO: 203) | GSGSGTDFTLTISSLEPEDFA | NO: 435) | | 207) |
| H10 L4 | KSISTAYLQWSSLKASDTA | | NO: 412) | | VYYCQQGQSYPLTFGQGTKL | | | |
| | MYYCARPLYSYASYYWFAY | | | | EIK | | | |
| | WGQGTLVTVSS | | | | (SEQ ID NO: 434) | | | |
| | (SEQ ID NO: 490) | | | | | | | |
| hACI- | EVQLVQSGAEVKKPGESLK | DQTIH | YIYPGDGS | PLYSYASYY | EIVLTQSPATLSLSPGERATLS | HASQNINV | KSSNLHT | QQGQSYPL |
| 8016- | ISCKGSGYSFTDQTIHWVH | (SEQ ID | AMYNEKF | WFAY | CHASQNINVYLSWYQQKPG | YLS | (SEQ ID | T |
| 32B6C7- | QMPGKGLEWMGYIYPGD | NO: 201) | QG | (SEQ ID | QAPRLLIYKSSNLHTGIPARFS | (SEQ ID | NO: 206) | (SEQ ID NO: |
| AB1 | GSAMYNEKFQGQVTISAD | | (SEQ ID | NO: 203) | GSGSGTDFTLTISSLEPEDFA | NO: 435) | | 207) |
| H11 L4 | KSISTAYLQWSSLKASDTA | | NO: 412) | | VYYCQQGQSYPLTFGQGTKL | | | |
| | MYYCARPLYSYASYYWFAY | | | | EIK | | | |
| | WGQGTLVTVSS | | | | (SEQ ID NO: 434) | | | |
| | (SEQ ID NO: 500) | | | | | | | |
| hACI- | EVQLVQSGAEVKKPGESLK | DQTIH | YIYPGDSSA | PLYSYASYY | EIVLTQSPATLSLSPGERATLS | HASQNINV | KSSNLHT | QQGQSYPL |
| 8016- | ISCKISGYTFTDQTIHWVH | (SEQ ID | MYNEKFQ | WFAY | CHASQNINVYLSWYQQKPG | YLS | (SEQ ID | T |
| 32B6C7- | QMPGKGLEWMGYIYPGD | NO: 201) | G | (SEQ ID | QAPRLLIYKSSNLHTGIPARFS | (SEQ ID | NO: 206) | (SEQ ID NO: |
| AB1 | SSAMYNEKFQGQVTISAD | | (SEQ ID | NO: 203) | GSGSGTDFTLTISSLEPEDFA | NO: 435) | | 207) |
| H12 L4 | KSISTAYLOWSSLKASDTA | | NO: 462) | | VYYCQQGQSYPLTFGQGTKL | | | |
| | MYYCARPLYSYASYYWFAY | | | | EIK | | | |
| | WGQGTLVTVSS | | | | (SEQ ID NO: 434) | | | |
| | (SEQ ID NO: 510) | | | | | | | |
| hACI- | EVQLVQSGAEVKKPGESLK | DQTIH | YIYPGDSSA | PLYSYASYY | EIVLTQSPATLSLSPGERATLS | HASQNINV | KSSNLHT | QQGQSYPL |
| 8016- | ISCKISGYSFTDQTIHWVH | (SEQ ID | MYNEKFQ | WFAY | CHASQNINVYLSWYQQKPG | YLS | (SEQ ID | T |
| 32B6C7- | QMPGKGLEWMGYIYPGD | NO: 201) | G | (SEQ ID | QAPRLLIYKSSNLHTGIPARFS | (SEQ ID | NO: 206) | (SEQ ID NO: |
| AB1 | SSAMYNEKFQGQVTISAD | | (SEQ ID | NO: 203) | GSGSGTDFTLTISSLEPEDFA | NO: 435) | | 207) |
| H13 L4 | KSISTAYLOWSSLKASDTA | | NO: 462) | | VYYCQQGQSYPLTFGQGTKL | | | |
| | MYYCARPLYSYASYYWFAY | | | | EIK | | | |
| | WGQGTLVTVSS | | | | (SEQ ID NO: 434) | | | |
| | (SEQ ID NO: 520) | | | | | | | |
| hACI- | QVQLVQSGAEVKKPGSSV | DQTIH | YIYPGDSSA | PLYSYASYY | EIVLTQSPATLSLSPGERATLS | HASQNINV | KSSNLHT | QQGQSYPL |
| 8016- | KVSCKASGYTFTDQTIHWV | (SEQ ID | MYNEKFQ | WFAY | CHASQNINVWLSWYQQKP | WLS | (SEQ ID | T |
| 32B6C7- | RQAPGQGLEWMGYIYPG | NO: 201) | G | (SEQ ID | GQAPRLLIYKSSNLHTGIPAR | (SEQ ID | NO: 206) | (SEQ ID NO: |
| AB1 | DSSAMYNEKFQGRVTITA | | (SEQ ID | NO: 203) | FSGSGSGTDFTLTISSLEPEDF | NO: 205) | | 207) |
| H14L3 | DKSTSTAYMELSSLRSEDT | | NO: 462) | | AVYYCQQGQSYPLTFGQGT | | | |
| | AVYYCARPLYSYASYYWFA | | | | KLEIK | | | |
| | YWGQGTLVTVSS | | | | (SEQ ID NO: 424) | | | |
| | (SEQ ID NO: 530) | | | | | | | |
| hACI- | QVQLVQSGAEVKKPGSSV | DQTIH | YIYPGDAS | PLYSYASYY | EIVLTQSPATLSLSPGERATLS | HASQNINV | KSSNLHT | QQGQSYPL |
| 8016- | KVSCKASGYTFTDQTIHWV | (SEQ ID | AMYNEKF | WFAY | CHASQNINVWLSWYQQKP | WLS | (SEQ ID | T |
| 32B6C7- | RQAPGQGLEWMGYIYPG | NO: 201) | QG | (SEQ ID | GQAPRLLIYKSSNLHTGIPAR | (SEQ ID | NO: 206) | (SEQ ID NO: |
| AB1 | DASAMYNEKFQGRVTITA | | (SEQ ID | NO: 203) | FSGSGSGTDFTLTISSLEPEDF | NO: 205) | | 207) |
| H15L3 | DKSTSTAYMELSSLRSEDT | | NO: 472) | | AVYYCQQGQSYPLTFGQGT | | | |
| | AVYYCARPLYSYASYYWFA | | | | KLEIK | | | |
| | YWGQGTLVTVSS | | | | (SEQ ID NO: 424) | | | |
| | (SEQ ID NO: 540) | | | | | | | |
| hACI- | EVQLVQSGAEVKKPGESLK | DQTIH | YIYPGDGS | PLYSYASYY | EIVLTQSPATLSLSPGERATLS | HASQNINV | KSSNLHT | QQGQSYPL |
| 8016- | ISCKISGYTFTDQTIHWMH | (SEQ ID | AMYNEKF | WFAY | CHASQNINVWLSWYQRKP | WLS | (SEQ ID | T |
| 32B6C7- | QMPGKGLEWIGYIYPGDG | NO: 201) | RG | (SEQ ID | GQAPKLLIYKSSNLHTGIPAR | (SEQ ID | NO: 206) | (SEQ ID NO: |
| AB1 | SAMYNEKFRGQATLSADK | | (SEQ ID | NO: 203) | FSGSGSGTDFTLTISSLEPEDF | NO: 205) | | 207) |
| H1L2 | SISTAYLQWSSLKASDTAM | | NO: 202) | | AVYYCQQGQSYPLTFGQGT | | | |
| | YFCARPLYSYASYYWFAYW | | | | KLEIK | | | |
| | GQGTLVTVSS | | | | (SEQ ID NO: 414) | | | |
| | (SEQ ID NO: 400) | | | | | | | |
| hACI- | EVQLVQSGAEVKKPGESLK | DQTIH | YIYPGDGS | PLYSYASYY | EIVLTQSPATLSLSPGERATLS | HASQNINV | KSSNLHT | QQGQSYPL |
| 8016- | ISCKISGYTFTDQTIHWMH | (SEQ ID | AMYNEKF | WFAY | CHASQNINVWLSWYQQKP | WLS | (SEQ ID | T |
| 32B6C7- | QMPGKGLEWIGYIYPGDG | NO: 201) | RG | (SEQ ID | GQAPRLLIYKSSNLHTGIPAR | (SEQ ID | NO: 206) | (SEQ ID NO: |
| AB1 | SAMYNEKFRGQATLSADK | | (SEQ ID | NO: 203) | FSGSGSGTDFTLTISSLEPEDF | NO: 205) | | 207) |
| H1L3 | SISTAYLQWSSLKASDTAM | | NO: 202) | | AVYYCQQGQSYPLTFGQGT | | | |
| | YFCARPLYSYASYYWFAYW | | | | KLEIK | | | |
| | GQGTLVTVSS | | | | (SEQ ID NO: 424) | | | |
| | (SEQ ID NO: 400) | | | | | | | |
| hACI- | EVQLVQSGAEVKKPGESLK | DQTIH | YIYPGDGS | PLYSYASYY | DIQMTQSPSTLSASVGDRVT | HASQNINV | KSSNLHT | QQGQSYPL |
| 8016- | ISCKISGYTFTDQTIHWVH | (SEQ ID | AMYNEKF | WFAY | ITCHASQNINVWLSWYQRK | WLS | (SEQ ID | T |
| 32B6C7- | QMPGKGLEWMGYIYPGD | NO: 201) | QG | (SEQ ID | PGKAPKLLIYKSSNLHTGVPS | (SEQ ID | NO: 206) | (SEQ ID NO: |
| AB1 | GSAMYNEKFQGQVTISAD | | (SEQ ID | NO: 203) | RFSGSGSGTEFTLTISSLQPD | NO: 205) | | 207) |
| H2L1 | KSISTAYLQWSSLKASDTA | | NO: 412) | | DFATYYCQQGQSYPLTFGQ | | | |
| | MYYCARPLYSYASYYWFAY | | | | GTKLEIK | | | |
| | WGQGTLVTVSS | | | | (SEQ ID NO: 404) | | | |
| | (SEQ ID NO: 410) | | | | | | | |
| hACI- | EVQLVQSGAEVKKPGESLK | DQTIH | YIYPGDGS | PLYSYASYY | EIVLTQSPATLSLSPGERATLS | HASQNINV | KSSNLHT | QQGQSYPL |
| 8016- | ISCKISGYTFTDQTIHWVH | (SEQ ID | AMYNEKF | WFAY | CHASQNINVWLSWYQQKP | WLS | (SEQ ID | T |
| 32B6C7- | QMPGKGLEWMGYIYPGD | NO: 201) | QG | (SEQ ID | GQAPRLLIYKSSNLHTGIPAR | (SEQ ID | NO: 206) | (SEQ ID NO: |
| AB1 | GSAMYNEKFQGQVTISAD | | (SEQ ID | NO: 203) | FSGSGSGTDFTLTISSLEPEDF | NO: 205) | | 207) |
| H2L3 | KSISTAYLQWSSLKASDTA | | NO: 412) | | AVYYCQQGQSYPLTFGQGT | | | |
| | MYYCARPLYSYASYYWFAY | | | | KLEIK | | | |
| | WGQGTLVTVSS | | | | (SEQ ID NO: 424) | | | |
| | (SEQ ID NO: 410) | | | | | | | |
| hACI- | EVOLVQSGAEVKKPGESLK | DQTIH | YIYPGDGS | PLYSYASYY | EIVLTQSPATLSLSPGERATLS | HASQNINV | KSSNLHT | QQGQSYPL |
| 8016- | ISCKISGYTFTDQTIHWVH | (SEQ ID | AMYNEKF | WFAY | CHASQNINVYLSWYQQKPG | YLS | (SEQ ID | T |
| 32B6C7- | QMPGKGLEWMGYIYPGD | NO: 201) | QG | (SEQ ID | QAPRLLIYKSSNLHTGIPARFS | (SEQ ID | NO: 206) | (SEQ ID NO: |
| AB1 | GSAMYNEKFQGQVTISAD | | (SEQ ID | NO: 203) | GSGSGTDFTLTISSLEPEDFA | NO: 435) | | 207) |
| H2L4 | KSISTAYLQWSSLKASDTA | | NO: 412) | | VYYCQQGQSYPLTFGQGTKL | | | |
| | MYYCARPLYSYASYYWFAY | | | | EIK | | | |
| | WGQGTLVTVSS | | | | (SEQ ID NO: 434) | | | |
| | (SEQ ID NO: 410) | | | | | | | |
| hACI- | QVQLVQSGAEVKKPGSSV | DQTIH | YIYPGDGS | PLYSYASYY | DIQMTQSPSTLSASVGDRVT | HASQNINV | KSSNLHT | QQGQSYPL |
| 8016- | KVSCKISGYSFTDQTIHWM | (SEQ ID | AMYNEKF | WFAY | ITCHASQNINVWLSWYQRK | WLS | (SEQ ID | T |
| 32B6C7- | RQAPGQGLEWIGYIYPGD | NO: 201) | RG | (SEQ ID | PGKAPKLLIYKSSNLHTGVPS | (SEQ ID | NO: 206) | (SEQ ID NO: |
| AB1 | GSAMYNEKFRGRATLTAD | | (SEQ ID | NO: 203) | RFSGSGSGTEFTLTISSLQPD | NO: 205) | | 207) |
| H3L1 | KSTSTAYMELSSLRSEDTAV | | NO: 202) | | DFATYYCQQGQSYPLTFGQ | | | |
| | YFCARPLYSYASYYWFAYW | | | | GTKLEIK | | | |
| | GQGTLVTVSS | | | | (SEQ ID NO: 404) | | | |
| | (SEQ ID NO: 420) | | | | | | | |
| hACI- | QVQLVQSGAEVKKPGSSV | DQTIH | YIYPGDGS | PLYSYASYY | EIVLTQSPATLSLSPGERATLS | HASQNINV | KSSNLHT | QQGQSYPL |
| 8016- | KVSCKISGYSFTDQTIHWM | (SEQ ID | AMYNEKF | WFAY | CHASQNINVWLSWYQRKP | WLS | (SEQ ID | T |
| 32B6C7- | RQAPGQGLEWIGYIYPGD | NO: 201) | RG | (SEQ ID | GQAPKLLIYKSSNLHTGIPAR | (SEQ ID | NO: 206) | (SEQ ID NO: |
| AB1 | GSAMYNEKFRGRATLTAD | | (SEQ ID | NO: 203) | FSGSGSGTDFTLTISSLEPEDF | NO: 205) | | 207) |
| H3L2 | KSTSTAYMELSSLRSEDTAV | | NO: 202) | | AVYYCQQGQSYPLTFGQGT | | | |
| | YFCARPLYSYASYYWFAYW | | | | KLEIK | | | |
| | GQGTLVTVSS | | | | (SEQ ID NO: 414) | | | |
| | (SEQ ID NO: 420) | | | | | | | |
| hACI- | QVQLVQSGAEVKKPGSSV | DQTIH | YIYPGDGS | PLYSYASYY | DIQMTQSPSTLSASVGDRVT | HASQNINV | KSSNLHT | QQGQSYPL |
| 8016- | KVSCKISGYSFTDQTIHWV | (SEQ ID | AMYNEKF | WFAY | ITCHASQNINVWLSWYQRK | WLS | (SEQ ID | T |
| 32B6C7- | RQAPGQGLEWMGYIYPG | NO: 201) | QG | (SEQ ID | PGKAPKLLIYKSSNLHTGVPS | (SEQ ID | NO: 206) | (SEQ ID NO: |
| AB1 | DGSAMYNEKFQGRVTITA | | (SEQ ID | NO: 203) | RFSGSGSGTEFTLTISSLQPD | NO: 205) | | 207) |
| H4L1 | DKSTSTAYMELSSLRSEDT | | NO: 412) | | DFATYYCQQGQSYPLTFGQ | | | |
| | AVYYCARPLYSYASYYWFA | | | | GTKLEIK | | | |
| | YWGQGTLVTVSS | | | | (SEQ ID NO: 404) | | | |
| | (SEQ ID NO: 430) | | | | | | | |
| hACI- | QVQLVQSGAEVKKPGSSV | DQTIH | YIYPGDGS | PLYSYASYY | EIVLTQSPATLSLSPGERATLS | HASQNINV | KSSNLHT | QQGQSYPL |
| 8016- | KVSCKISGYSFTDQTIHWV | (SEQ ID | AMYNEKF | WFAY | CHASQNINVWLSWYQRKP | WLS | (SEQ ID | T |
| 32B6C7- | RQAPGQGLEWMGYIYPG | NO: 201) | QG | (SEQ ID | GQAPKLLIYKSSNLHTGIPAR | (SEQ ID | NO: 206) | (SEQ ID NO: |
| AB1 | DGSAMYNEKFQGRVTITA | | (SEQ ID | NO: 203) | FSGSGSGTDFTLTISSLEPEDF | NO: 205) | | 207) |
| H4L2 | DKSTSTAYMELSSLRSEDT | | NO: 412) | | AVYYCQQGQSYPLTFGQGT | | | |
| | AVYYCARPLYSYASYYWFA | | | | KLEIK | | | |
| | YWGQGTLVTVSS | | | | (SEQ ID NO: 414) | | | |
| | (SEQ ID NO: 430) | | | | | | | |
| hACI- | QVQLVQSGAEVKKPGSSV | DQTIH | YIYPGDGS | PLYSYASYY | EIVLTQSPATLSLSPGERATLS | HASQNINV | KSSNLHT | QQGQSYPL |
| 8016- | KVSCKISGYSFTDQTIHWV | (SEQ ID | AMYNEKF | WFAY | CHASQNINVWLSWYQQKP | WLS | (SEQ ID | T |
| 32B6C7- | RQAPGQGLEWMGYIYPG | NO: 201) | QG | (SEQ ID | GQAPRLLIYKSSNLHTGIPAR | (SEQ ID | NO: 206) | (SEQ ID NO: |
| AB1 | DGSAMYNEKFQGRVTITA | | (SEQ ID | NO: 203) | FSGSGSGTDFTLTISSLEPEDF | NO: 205) | | 207) |
| H4L3 | DKSTSTAYMELSSLRSEDT | | NO: 412) | | AVYYCQQGQSYPLTFGQGT | | | |
| | AVYYCARPLYSYASYYWFA | | | | KLEIK | | | |
| | YWGQGTLVTVSS | | | | (SEQ ID NO: 424) | | | |
| | (SEQ ID NO: 430) | | | | | | | |
| hACI- | QVQLVQSGAEVKKPGSSV | DQTIH | YIYPGDGS | PLYSYASYY | EIVLTQSPATLSLSPGERATLS | HASQNINV | KSSNLHT | QQGQSYPL |
| 8016- | KVSCKASGYTFTDQTIHWV | (SEQ ID | AMYNEKF | WFAY | CHASQNINVWLSWYQQKP | WLS | (SEQ ID | T |
| 32B6C7- | RQAPGQGLEWMGYIYPG | NO: 201) | QG | (SEQ ID | GQAPRLLIYKSSNLHTGIPAR | (SEQ ID | NO: 206) | (SEQ ID NO: |
| AB1 | DGSAMYNEKFQGRVTITA | | (SEQ ID | NO: 203) | FSGSGSGTDFTLTISSLEPEDF | NO: 205) | | 207) |
| H6L3 | DKSTSTAYMELSSLRSEDT | | NO: 412) | | AVYYCQQGQSYPLTFGQGT | | | |
| | AVYYCARPLYSYASYYWFA | | | | KLEIK | | | |
| | YWGQGTLVTVSS | | | | (SEQ ID NO: 424) | | | |
| | (SEQ ID NO: 450) | | | | | | | |
| hACI- | EVQLVQSGAEVKKPGESLK | DQTIH | YIYPGDGS | PLYSYASYY | EIVLTQSPATLSLSPGERATLS | HASQNINV | KSSNLHT | QQGQSYPL |
| 8016- | ISCKISGYSFTDQTIHWVH | (SEQ ID | AMYNEKF | WFAY | CHASQNINVWLSWYQQKP | WLS | (SEQ ID | T |
| 32B6C7- | QMPGKGLEWMGYIYPGD | NO: 201) | QG | (SEQ ID | GQAPRLLIYKSSNLHTGIPAR | (SEQ ID | NO: 206) | (SEQ ID NO: |
| AB1 | GSAMYNEKFQGQVTISAD | | (SEQ ID | NO: 203) | FSGSGSGTDFTLTISSLEPEDF | NO: 205) | | 207) |
| H9 L3 | KSISTAYLQWSSLKASDTA | | NO:412) | | AVYYCQQGQSYPLTFGQGT | | | |
| | MYYCARPLYSYASYYWFAY | | | | KLEIK | | | |
| | WGQGTLVTVSS | | | | (SEQ ID NO: 424) | | | |
| | (SEQ ID NO: 480) | | | | | | | |
| hACI- | EVQLVQSGAEVKKPGESLK | DQTIH | YIYPGDSSA | PLYSYASYY | EIVLTQSPATLSLSPGERATLS | HASQNINV | KSSNLHT | QQGQSYPL |
| 8016- | ISCKISGYSFTDQTIHWVH | (SEQ ID | MYNEKFQ | WFAY | CHASQNINVWLSWYQQKP | WLS | (SEQ ID | T |
| 32B6C7- | QMPGKGLEWMGYIYPGD | NO: 201) | G | (SEQ ID | GQAPRLLIYKSSNLHTGIPAR | (SEQ ID | NO: 206) | (SEQ ID NO: |
| AB1 | SSAMYNEKFQGQVTISAD | | (SEQ ID | NO: 203) | FSGSGSGTDFTLTISSLEPEDF | NO: 205) | | 207) |
| H13 L3 | KSISTAYLQWSSLKASDTA | | NO: 462) | | AVYYCQQGQSYPLTFGQGT | | | |
| | MYYCARPLYSYASYYWFAY | | | | KLEIK | | | |
| | WGQGTLVTVSS | | | | (SEQ ID NO: 424) | | | |
| | (SEQ ID NO: 520) | | | | | | | |

### Production of Humanized Antibody Variants

DNA coding sequence for both heavy and light variable domains were synthesized and cloned using standard molecular biology techniques into plasmid allowing the expression in mammalian cells. Heavy chain variable domains were fused to the human IgG1 constant domain and light chain variable domains were cloned into plasmid containing the constant Kappa light chain domain. The chimeric antibody and the humanized variants were transiently expressed in ExpiCHO-S (ThermoFischer scientific, A29127) cells by cotransfecting heavy and light chain plasmid using the ExpiCHO™ Expression System Kit (ThermoFischer scientific, A29133). Post transfection, cells were maintained at 37° C. under 150 rpm agitation and 8% C02 level. Six days after transfection, supernatants were harvested and purified by protein A (Cytiva, 17127903). Antibodies were captured by incubation with protein A at room temperature for 2 hours and under agitation on rollers. The mixture was poured into a gravity flow chromatography column (BioRad, 7321010), the resin was washed with 10CV of 2×PBS and eluted with 0.1M Glycine pH3.2. The elution was then neutralized by adding 0.1 M Tris, pH 7.4. The samples were then dialyzed in PBS buffer.

### Characterization of ACI-8016-32B6C7-AB1 Humanized IgG Variants by Surface Plasmon Resonance (SPR)

All humanized antibodies were screened for binding to human ASC and the affinity of the best humanized variants measured by Surface Plasmon Resonance (SPR).

First, the affinity to human ASC of ACI-8016-32B6C7-AB1 humanized IgG variants was measured by capturing the anti-ASC antibodies at the surface of a sensor chip. However, measurements reached the upper limit of sensitivity for a Biacore 8K instrument and accurate affinity could not be calculated. Therefore, all humanized antibodies and the chimeric antibody were reformatted as Fabs to perform measurement on human ASC immobilized on the sensor chip and reduce the avidity effect caused by the presence of human ASC multimers.

Affinity measurements were performed on a Biacore 8K instrument (Cytiva, formerly GE Healthcare) using a single-cycle kinetics method.

The instrument was primed with running buffer (10×PBS-P+ diluted to 1× in Milli-Q water). Flow-cells 1-2 of all 8 Fc,s on a CM5 Series S sensor chip were activated with a fresh solution of EDC/NHS at 10 μL/min for 420 sec (Amine Coupling Kit, 1:1 ratio both reagents). hASC was diluted in 10 mM sodium acetate pH 4.0 to a final concentration of 50 μg/mL and injected for 600 sec on Fc2 of all 8 Fc to a final level of 500 RU. Next, all unreacted activated ester groups were quenched with 1 M ethanolamine for 420 sec. Two successive regenerations of 10 mM glycine-HCl pH 1.7 for 30 sec were conducted to remove any non-covalently bound ASC.

Single-cycle kinetics were performed with a surface regeneration between each cycle. Prior to analysis, two Startup Cycles were run. ACI-8016-32B6C7-AB1 humanized Fabs were injected in increasing concentration from 1.2-100 nM, prepared from a 3-fold serial dilution in running buffer, with a contact time of 300 sec and a dissociation time of 900 sec for the chimeric Fab and 3600 sec for the human Fab at a flow rate of 30 μL/min. Each successive Fab was preceded by a regeneration step using 10 mM glycine-HCl pH 1.7 with a contact time of 30 sec at 10 μL/min, followed by a stabilization period of 300 sec.

Results obtained from single-cycle kinetics were double-referenced using the blank flow-cell 1 and a buffer cycle and evaluated using the Biacore 8K evaluation software with 1:1 kinetic fit model (global Rmax). The following kinetic parameters were obtained: association rate constant (ka), dissociation rate constant (kd), equilibrium dissociation constant (KD) and saturation response (Rmax).

All parameters (except Rmax) are reported in Table 21 as mean from 2 independent experiments.

TABLE 21
ka, kd, KD value of ACI-8016-32B6C7-
AB1 humanized Fab variants on human ASC

| | ka | kd | KD |
|---|---|---|---|
| mAb | (1/Ms) | (1/s) | (pM) |
| ACI-8016-32B6C7-AB1* | 2.09E+04 | 2.11E−05 | 1010 |
| hACI-8016-32B6C7-AB1_H2L3 | 8.22E+04 | 8.64E−06 | 105 |
| hACI-8016-32B6C7-AB1_H4L3 | 1.08E+05 | 8.16E−06 | 77 |
| hACI-8016-32B6C7-AB1_H5L4 | 8.45E+04 | 6.68E−06 | 79 |
| hACI-8016-32B6C7-AB1_H6L4 | 7.92E+04 | 5.28E−06 | 67 |
| hACI-8016-32B6C7-AB1_H7L4 | 8.43E+04 | 5.62E−06 | 67 |
| hACI-8016-32B6C7-AB1_H8L4 | 9.40E+04 | 7.03E−06 | 77 |
| hACI-8016-32B6C7-AB1_H9L4 | 9.20E+04 | 7.52E−06 | 81 |
| hACI-8016-32B6C7-AB1_H13L4 | 8.41E+04 | 7.09E−06 | 84 |
| hACI-8016-32B6C7-AB1_H6L3 | 8.72E+04 | 7.52E−06 | 86 |
| hACI-8016-32B6C7-AB1_H9L3 | 9.91E+04 | 8.87E−06 | 91 |
| hACI-8016-32B6C7-AB1_H13L3 | 1.05E+05 | 8.01E−06 | 77 |
| hACI-8016-32B6C7-AB1_H14L3 | 1.01E+05 | 4.96E−06 | 49 |
| hACI-8016-32B6C7-AB1_H15L3 | 1.03E+05 | 4.82E−06 | 52 |

Overall, all humanized variants show similar or superior affinity to human ASC as observed for the chimeric antibody, ACI-8016-32B6C7-AB1.

### Example 12: Epitope Mapping Via Alanine Scanning Mutagenesis: PYD Domain

### Methods

Alanine mutants of PYD domain of PYCARD were recombinantly produced with His and MBP tags in N and C terminus respectively. Doublets of alanine mutants were introduced sequentially at position exposed to solvent according to PDB 1UCP. A total of 30 alanine mutants were designed and cloned into high copy expression vector. Bacterial cultures were induced at OD600=0.8 with 0.5 mM IPTG and cultivated at 28° C. for 16 h in TB medium supplemented with kanamycin (final concentration 50 μg/mL). 15 mL of overnight culture were centrifuged at 8 000×g, for 10 min at 4° C., bacterial pellets were resuspended in 3 mL of Lysis Buffer 50 mM Tris-HCl pH 8.0, 500 mM NaCl, 1 mM TECP, protease inhibitor cocktail (1000×dil.), DNAse (1000×dil.) and subjected to cells disruption using the Bioruptor® Plus sonication device (Diagenode, 2×: 20 sec on, 20 sec off, 8 min). Soluble fractions were frozen in liquid nitrogen and stored at −80° C.

ASC antibodies were tested for binding to alanine mutants by sandwich ELISA. Ninety-six well plates were coated overnight at 4° C. with 50 μl/well at 0.25 μg/ml of anti-MBP antibody (cat no: Ab01423-23.0, Absolute antibody). Wells were blocked 1 h at RT with 100 μl/well of 1×PBS 5% Milk. After incubation, wells were washed four times with 300 μl of washing buffer (1×PBS, 0.05% Tween 20). For each alanine mutant, 50 ml of a 1:2 dilution of E. coli crude extract in 1×PBS, 5% Milk, 0.05% Tween 20 were added to each well. After 1 h incubation at RT, plates were washed as previously described and 50 ml of anti-ASC mouse IgG2a at 0.5 pg/ml diluted in 1×PBS, 5% Milk, 0.05% Tween 20 were added to each well. After 1h incubation at RT, plates were washed as previously described. An anti-mouse IgG2a HRP secondary antibody (Abcam, ab97245) was diluted 1/10,000 in washing buffer and 50 ml added to each well. Plates were incubated for 1 h at RT and washed as previously described. TMB substrate (BD Biosciences, 555214) was prepared and 50 ml per well was added. Plates were incubated 10-15 min at RT. 50 ml of 2M HCL was added to each well to stop the reaction. Plates were read at 450 nm on a TECAN plate-reader. Antibody binding signal was normalized to the binding signal to the PYD-WT. An arbitrary threshold of 30% of binding was used to identify epitope critical residues.

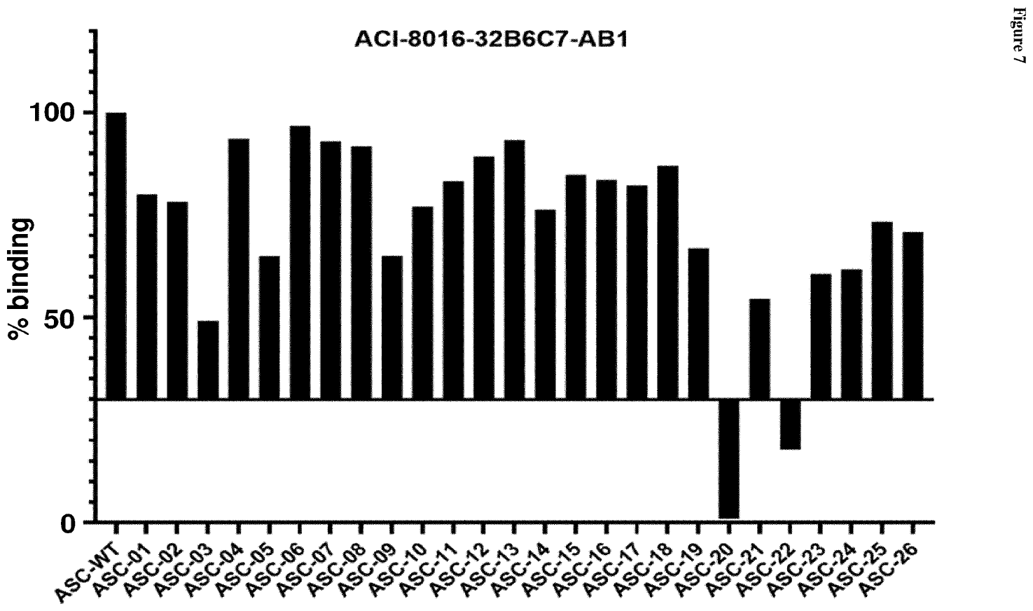

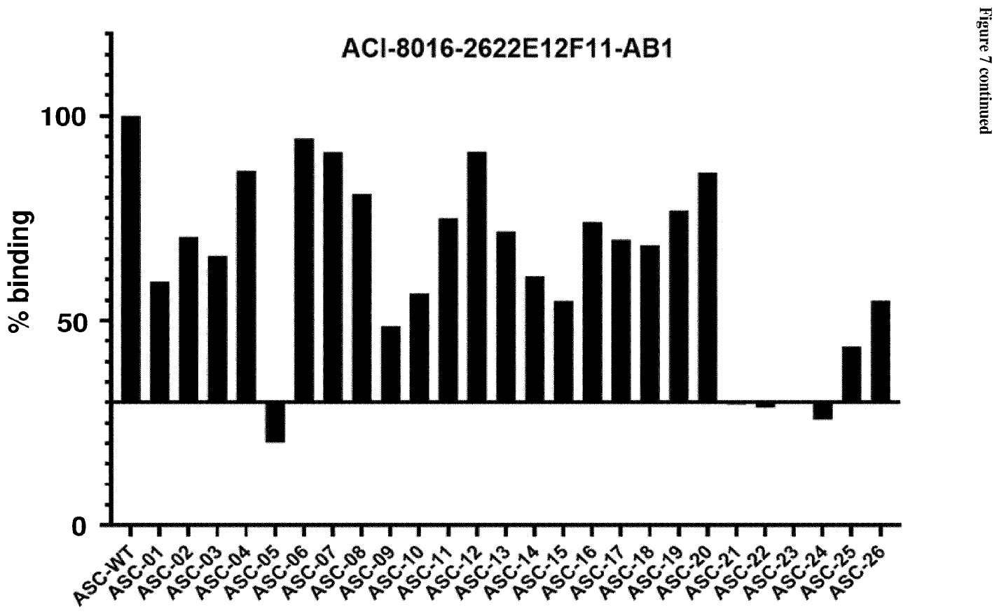

Results FIG. 6 shows the binding percentage to the different PYD mutants. Mutations showing a normalized binding of 30% or below represent the critical residue for target binding and define the antibody epitope. These residues critical for mAb binding are listed in Table 22. The correlation between mutants in FIG. 6 and mutated residues is shown in Table 123. These data correlate with the epitope binning experiment showing that ACI-8016-2626B9D3-AB1, ACI-8016-23E5F7-AB2, ACI-8016-26A1G2-AB1, ACI-8016-22D3A6-AB1, ACI-8016-2610H7D3-AB1 and ACI-8016-2617C3A8-AB1 have overlapping epitopes, different from the epitope of QACI-8016-401H97-AB1 and ACI-8016-2626B9D3-AB1.

TABLE 22
List of key/critical residues within the epitope
of PYD binding mAbs with reference to the amino
acid sequence of human ASC of SEQ ID NO: 1

| Antibody | Epitope |
|---|---|
| ACI-8016-2626B9D3-AB1 | L9/D10/E13/N14/E18/E19 |
| ACI-8016-23E5F7-AB2 | L9/D10/E13/N14/E18/E19 |
| ACI-8016-26A1G2-AB1 | L9/D10/E13/N14/E18/E19 |
| ACI-8016-22D3A6-AB1 | L9/D10/E13/N14 |
| ACI-8016-2610H7D3-AB1 | E13/N14 |
| ACI-8016-2617C3A8-AB1 | L9/D10/E13/N14 |
| ACI-8016-401H9B7-AB1 | Q79/E80/G83/Q84 |
| ACI-8016-2626B9D3-AB1 | E18/E19/V30/P31/N71/R74/ |
| | D75/G77/Q79/E80 |

TABLE 23
Correlation between PYD mutant name (FIG. 6) and amino
acid substitutions (with respect to SEQ ID NO:1)

| PYD mutant | Substitution |
|---|---|
| hPYD-01 | G2A/R3A |
| hPYD-02 | R5A/D6A |
| hPYD-03 | L9A/D10A |
| hPYD-04 | E13A/N14A |
| hPYD-06 | T16A |
| hPYD-07 | E18A/E19A |
| hPYD-08 | K21A/K22A |
| hPYD-09 | L25A/K26A |
| hPYD-10 | L28A/S29A |
| hPYD-11 | V30A/P31A |
| hPYD-12 | R33A/E34A |
| hPYD-13 | G35A/Y36A |
| hPYD-14 | G37A/R38A |
| hPYD-15 | P40A/R41A |
| hPYD-16 | G42A |
| hPYD-17 | L45A/S46A |
| hPYD-18 | D48A |
| hPYD-19 | L50A/D51A |
| hPYD-20 | D54A/K55A |
| hPYD-21 | S58A/F59A |
| hPYD-22 | L61A/E62A |
| hPYD-23 | T63A/Y64A |
| hPYD-24 | E67A |
| hPYD-25 | N71A |
| hPYD-26 | R74A/D75A |
| hPYD-27 | G77A |
| hPYD-28 | Q79A/E80A |
| hPYD-29 | G83A/Q84A |
| hPYD-30 | Q86A |
| hPYD-31 | H90A/Q91A |

### Example 13: Epitope Mapping Via Alanine Mutagenesis: CARD Domain

### Methods

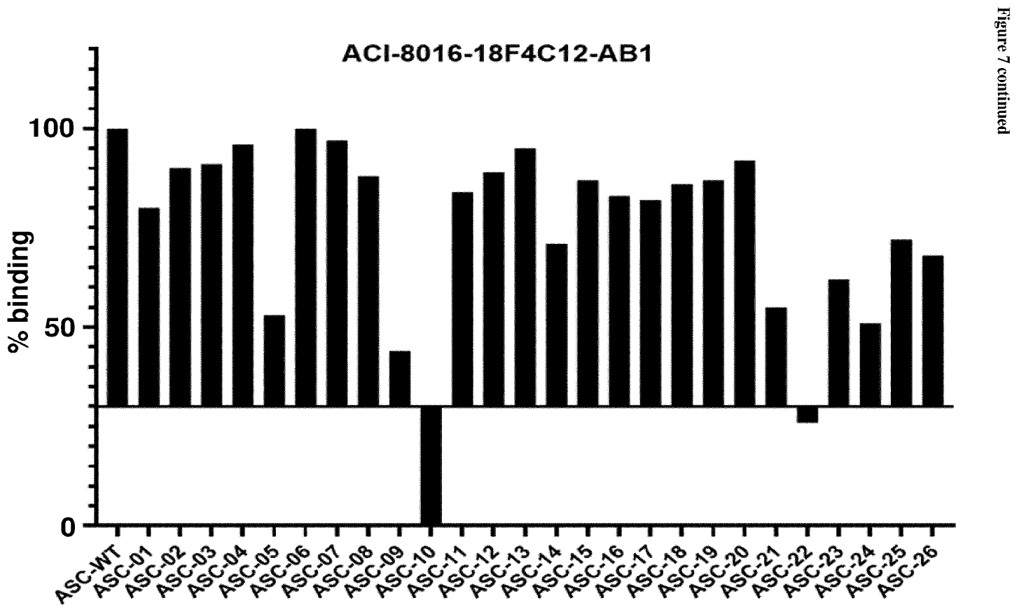

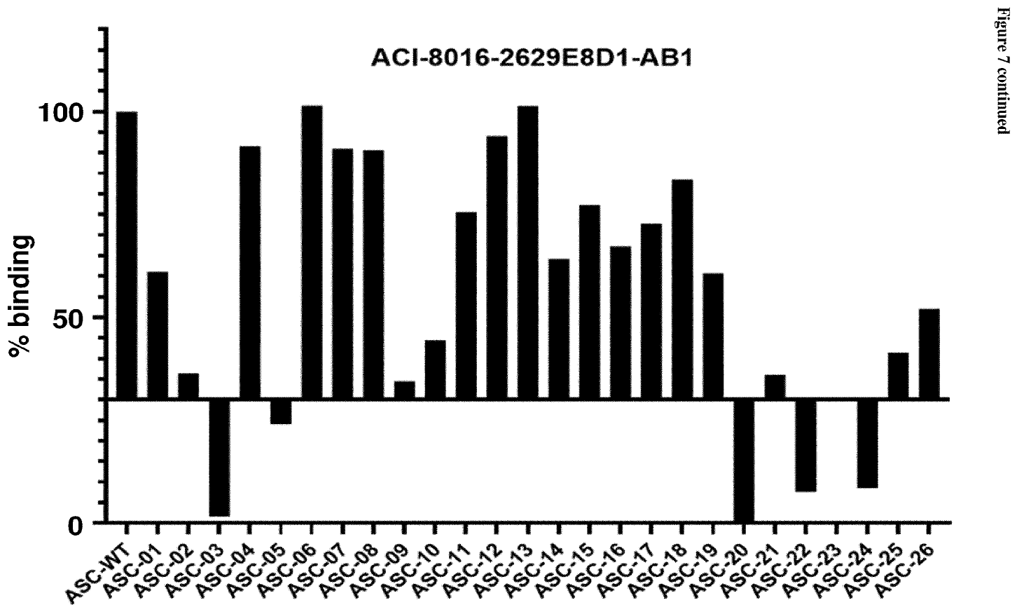

Alanine mutants of ASC were recombinantly produced with His and MBP tags in N and C terminus respectively. Based on structural analysis of PDB TUCP, single, doublets or triplets of alanine mutations were introduced sequentially at position exposed to solvent ranging from residue K109 to R194 on the human ASC sequence. Additionally, alanine 120 was mutated to a glutamine, its counterpart on the murine sequence of ASC. The correlation between mutants in FIG. 7 and mutated residues is shown in Table 25.

A total of 26 alanine mutants were designed and cloned into high copy expression vector. Bacterial cultures were induced at OD600=0.8 with 0.5 mM IPTG and cultivated at 28° C. for 16 h in TB medium supplemented with kanamycin (final concentration 50 μg/ml). 15 mL of overnight culture were centrifuged at 8 000×g, for 10 min at 4° C., bacterial pellets were resuspended in 3 mL of Lysis Buffer 50 mM Tris-HCl pH 8.0, 500 mM NaCl, 1 mM TECP, protease inhibitor cocktail (1000×dil.), DNAse (1000×dil.) and subjected to cells disruption. For example, cell disruption may be performed using the Bioruptor® Plus sonication device (Diagenode, 2×: 20 sec on, 20 sec off, 8 min). Soluble fractions were frozen in liquid nitrogen and stored at −80° C.

ASC antibodies were tested for binding to alanine mutants by sandwich ELISA. Ninety-six well plates were coated overnight at 4° C. with 50 μl/well at 2 μg/ml of anti-MBP antibody (cat no: Ab01423-23.0, Absolute antibody). Wells were blocked 1 h at RT with 100 l/well of 1×PBS 5% Milk. After incubation, wells were washed four times with 300 μl of washing buffer (1×PBS, 0.05% Tween 20). For each alanine mutant, 50 ml of a 1:2 dilution of E. coli crude extract in 1×PBS, 5% Milk, 0.05% Tween 20 were added to each well. After 1 h incubation at RT, plates were washed as previously described and 50 ml of anti-ASC mouse IgG2a at 2 μg/ml diluted in 1×PBS, 5% Milk, 0.05% Tween 20 were added to each well. After 1h incubation at RT, plates were washed as previously described. An anti-mouse IgG2a HRP secondary antibody (Abcam, ab97245) was diluted 1/10′000 in washing buffer and 50 ml added to each well. Plates were incubated for 1 h at RT and washed as previously described. TMB substrate (BD Biosciences, 555214) was prepared and 50 ml per well was added. Plates were incubated 10-15 min at RT. 50 ml of 2M HCL was added to each well to stop the reaction. Plates were read at 450 nm on a TECAN plate-reader. For each ASC mutants and each antibody, the binding signal was normalized to the binding signal to the ASC wild type construct. An arbitrary threshold of 30% of binding was used to identify epitope critical residues.

FIG. 7 shows the binding percentage to the different ASC mutants. Mutations showing a normalized binding of 30% or below, identify critical residues for target binding and define the antibody epitope. Residues critical for antibody binding are listed in Table 24.

TABLE 24
List of key/critical residues within the epitope
of CARD binding mAbs with reference to the amino
acid sequence of human ASC of SEQ ID NO: 1

| Antibody | Epitope |
|---|---|
| ACI-8016-32B6C7-AB1 | K174/D175/ |
| ACI-8016-2629E8D1-AB1 | I115/D116/R119/A120/K174/D175/ |
| | S184/Q185/S186/Y187 |
| ACI-8016-2504F3D9-AB1 | I115/D116/N170/W171/T172/K174/ |
| | D175/S186/Y187 |
| ACI-8016-18F4C12-AB1 | Y137 |
| ACI-8016-2622E12F11-AB1 | R119/A120/L178/Q179/S186/Y187 |
| ACI-8016-2609F4A9-AB1 | R119/A120/K174/D175/S186/Y187 |

TABLE 25
Correlation between CARD mutant name (FIG. 7) and amino
acid substitutions (with respect to SEQ ID NO:1)

| ASC mutants | Mutations |
|---|---|
| ASC-01 | K109A/P110A |
| ASC-02 | L112A/H113A |
| ASC-03 | I115A/D116A |
| ASC-04 | Q117A/H118A |
| ASC-05 | R119A/A120Q |
| ASC-06 | R125A |
| ASC-07 | T127A/N128A |
| ASC-08 | E130A/W131A |
| ASC-09 | D134A |
| ASC-10 | Y137A |
| ASC-11 | K139A/V140A |
| ASC-12 | T142/D143A |
| ASC-13 | E144A/Q145A/Q147A |
| ASC-14 | R150A/E152A/P153A |
| ASC-15 | T154A/N155A |
| ASC-16 | P156A/S157A |
| ASC-17 | R160A/K161A |
| ASC-18 | S164A/T166A |
| ASC-19 | N170A/W171A/T172A |
| ASC-20 | K174A/D175A |
| ASC-21 | L178A/Q179A |
| ASC-22 | R182A/E183A |
| ASC-23 | S184A/Q185A |
| ASC-24 | S186A/Y187A |
| ASC-25 | E190A/D191A |
| ASC-26 | E193A/R194A/S195A |

### Example 14: In Vivo Proof-of-Concept (PoC) Efficacy in a Demyelinating Mouse Model of NeuroInflammation (DMNI)

### Methods

Chronic demyelination was induced in female C57BL/6 mice (9-13 weeks old) by immunization with an emulsion of the short peptide myelin oligodendrocyte glycoprotein (MOG35-55)/Complete Freund's Adjuvant (CFA) followed by injection of pertussis toxin. Clinical symptoms were observed from 8 to 18 days after immunization. Experimental groups are defined in Table 26. All groups were dosed at 30 mg/kg i.p. on day 8, 11, and 13. All dosing was at the same time (+/−2 hours) each dosing day.

TABLE 26
Description of experimental groups

| Group | # mice | Immunization | Treatment |
|---|---|---|---|
| IgG2a Isotype | 12 | MOG35-55/CFA | IgG2a Isotype control |
| control | | | |
| mAb 1 | 12 | MOG35-55/CFA | ACI-8016-18F4C12-AB1 |
| mAb 2 | 12 | MOG35-55/CFA | ACI-8016-32B6C7-AB1 |
| IgG2a Isotype | 12 | PBS/CFA | IgG2a Isotype control |
| control (No | | | |
| DMNI) | | | |

### Clinical Score

Clinical score was evaluated on the scale 0 to 5 as shown in Table 27. All mice were scored daily starting from Day 7 until termination of the study on Day 17. Scoring was performed blind, by a person unaware of both treatment and of previous scores for each mouse. Body weight was measured 3 times/week, starting on Day 0. The Mean Maximum Score (MMS) was calculated by finding the maximum clinical score reached at any point during the study for each animal, and then taking the mean of these scores for the group.

TABLE 27
DMNI scoring criteria

| Score | Clinical observations |
|---|---|
| 0 | No obvious changes in motor functions of the |
| | mouse in comparison to non-immunized mice. |
| 1 | Limp tail. |
| 2 | Limp tail and weakness of hind legs. |
| 3 | Limp tail and complete paralysis of hind legs |
| | (most common); Or limp tail with paralysis of |
| | one front and one hind leg; Or all of them. |
| 4 | Limp tail, complete hind leg, and partial front |
| | leg paralysis. |
| 5 | Complete hind and complete front leg paralysis, |
| | no movement around the cage; Or mouse is |
| | spontaneously rolling in the cage; Or the mouse |
| | is found dead due to paralysis. |

Mice were euthanized on Day 17 and organs were collected. The spleen was collected, weighed, snap-frozen, and stored at −80° C. Spleen mass was calculated as % of bodyweight at the day of sacrifice.

### Histology

Spinal cords were collected and segments of cervical, thoracic, and lumbar spine were dissected and fixed in 10% formalin (for histological and immunohistochemical analysis).

Histological analysis was performed at day 17 after immunization. Demyelination was evaluated using anti-myelin basic protein (MBP) and scored as follows:
0—no demyelination (less than 2% demyelinated area)
1-2 to 5% demyelinated area
2-6 to 19% demyelinated area
3-20 to 29% demyelinated area
4-30 to 50% demyelinated area
5>50% demyelinated area

Three slides from each spinal cord were also stained and analyzed for Iba1, ASC, and CD4. The relative immunoreactive areas were evaluated by processing images with ImageJ software analysis and expressed as %.

### Biochemistry:

Detection of ASC and cleaved caspase-1 was performed in spinal cord tissues from DMNI mice by Capillary Electrophoresis using JESS western system. Mouse spinal cords (thoracic-lumbar section) were thawed on ice and homogenized with a mini handheld homogenizer in RIPA buffer (ThermoFisher) supplemented with protease inhibitors (complete EDTA-free protease inhibitors; Roche, 32524300) and protease Inhibitors (PhosSTOP phosphatase inhibitors, Roche, 4906837001) in 10 volumes of buffer to tissue weight. Samples were left on ice for 10 minutes before being centrifuged at 20′000g for 15 minutes at 4° C. on a benchtop centrifuge. The supernatant was aliquoted and stored at −80° C. The total protein concentration was determined using the Pierce™ BCA assay kit. Capillary electrophoresis was performed using Jess Simple Western system. Homogenized samples were diluted to 1 μg/l in PBS buffer then mixed with 5× master mix (reconstituted as per the supplier's instruction). Rabbit anti-ASC (Cell Signaling Technology, D2W8U) or mouse anti-cleaved caspase 1 (Adipogen, AG-20B-0042-C100) were used as primary antibody and diluted 1 in 50 in Milk-free antibody diluent (Bio-Techne, 043-524). The secondary antibodies used were the anti-rabbit and anti-mouse HRP (Anti-Rabbit Detection Module Chemiluminescence, Bio-Techne, DM-001 and Anti-Mouse Detection Module Chemiluminescence, Bio-Techne, DM-002).

### Results

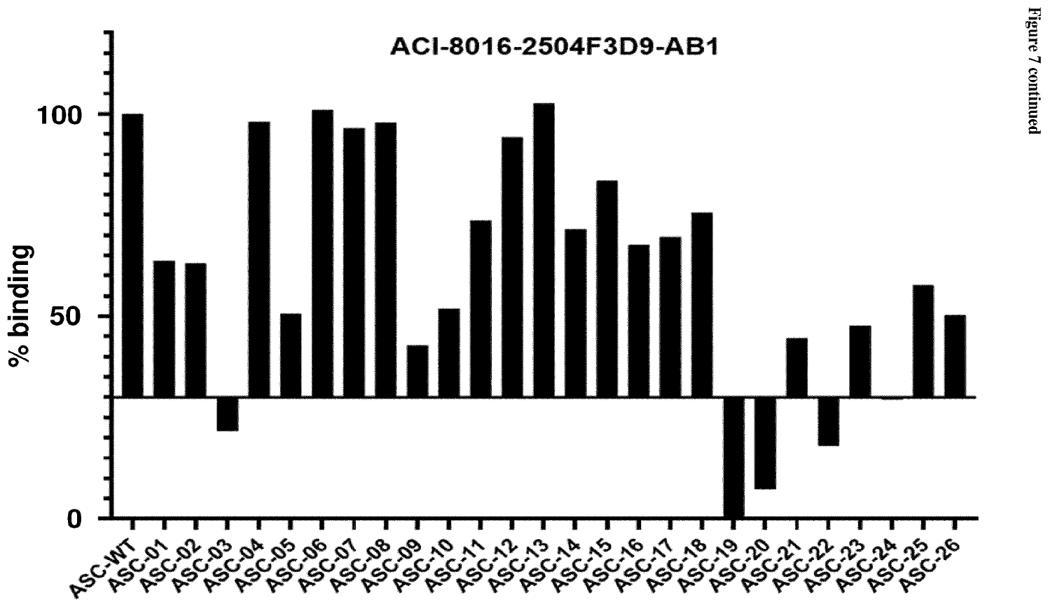

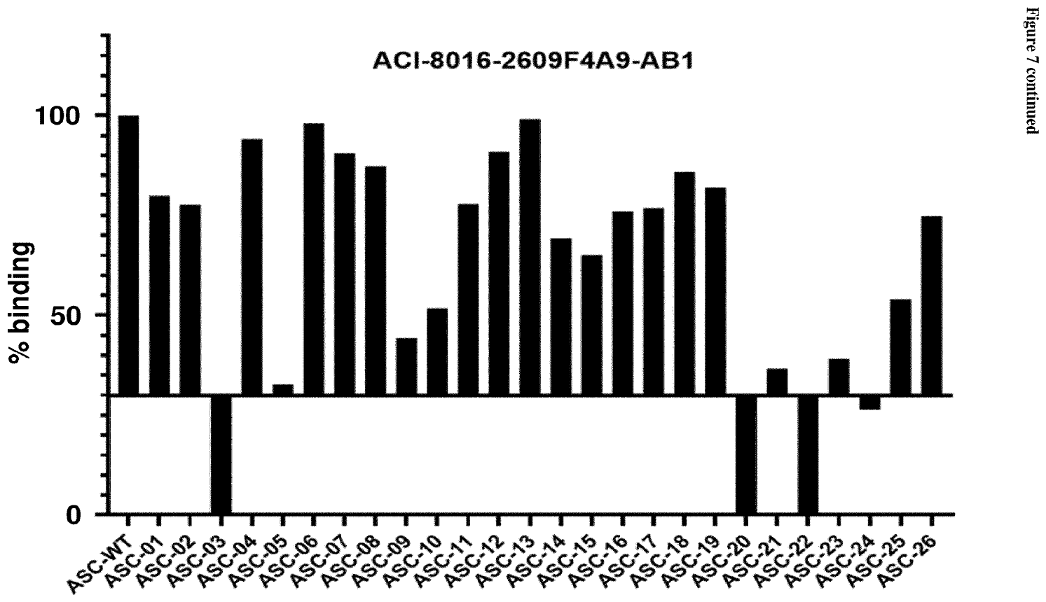

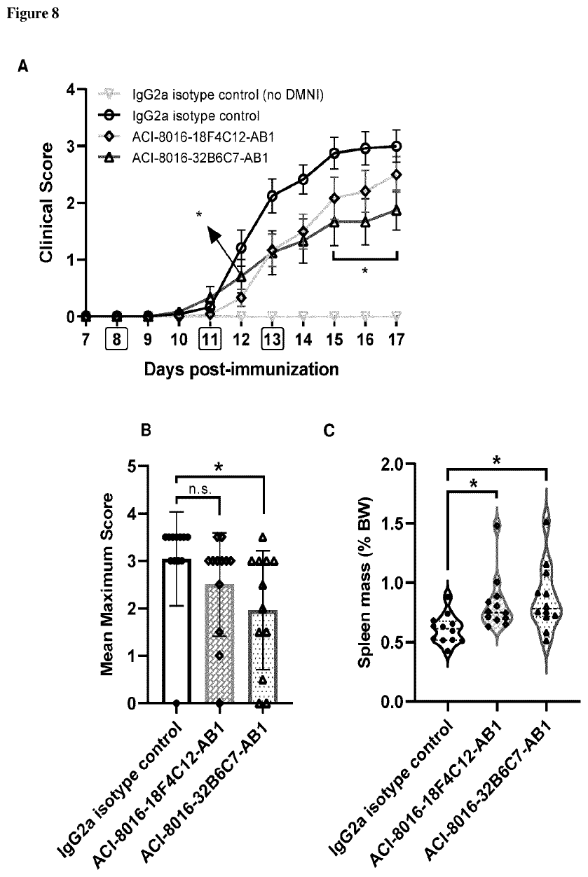

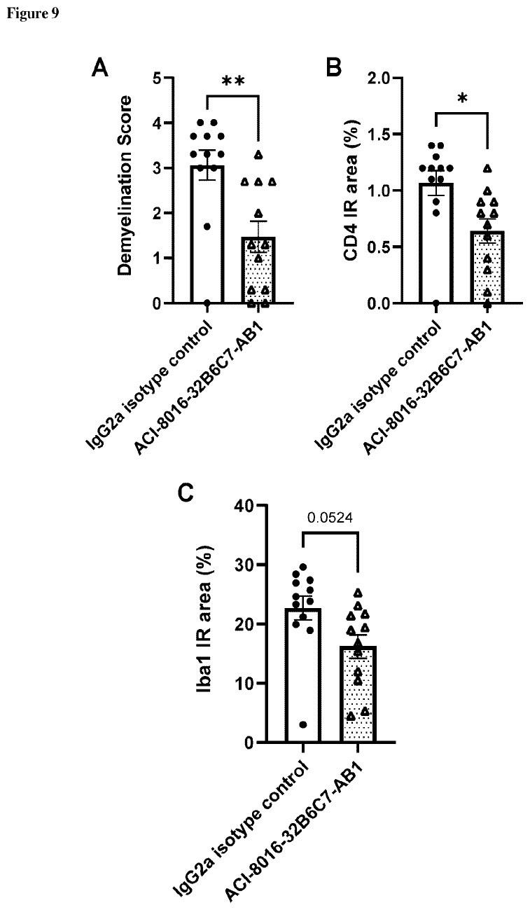

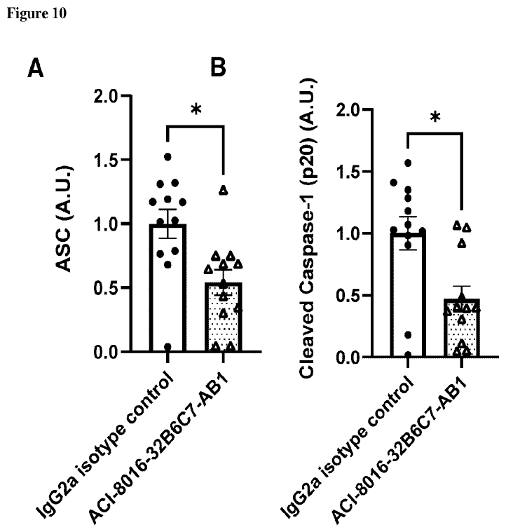

The effect of ASC mAbs on disease onset and progression was evaluated (FIG. 8). In the ACI-8016-32B6C7-AB1 group, DMNI onset was significantly postponed (FIG. 8A) as confirmed by a lower MMS score than the IgG2a isotype control group (FIG. 8B). Treatment with ACI-8016-18F4C12-AB1 marginally postponed the onset of the pathology as compared to the IgG2a isotype control group (FIG. 8A-B). Spleen mass was significantly increased in both ACI-8016-32B6C7-AB1 and ACI-8016-18F4C12-AB1 treated mice as compared to the IgG2a isotype control group (FIG. 8C) potentially indicating the ability of mAbs to limit cell egression from the spleen to the spinal cord as one of the triggers for neuroinflammation and ultimately demyelination. The amelioration of the clinical outcome by ACI-8016-32B6C7-AB1 was further corroborated by the improved demyelination score, (FIG. 9A), decreased presence of infiltrating T cells in the spinal cord (FIG. 9B), and substantial reduction in reactive microglia (FIG. 9C). Biochemical assessment of the inflammasome-related proteins in spinal cord lysates showed a significant reduction in ASC and cleaved caspase-1 levels in mice given ACI-8016-32B6C7-AB1 as compared to the Ig2a isotype control group (FIG. 10A-B). Taken together, these in vivo findings demonstrate that binding and inhibiting ASC function via mAbs can exert therapeutic activity by blocking inflammatory mechanisms responsible for the demyelinating disease.

### REFERENCES

Adamczak, S., G. Dale, J. P. de Rivero Vaccari, M. R. Bullock, W. D. Dietrich and R. W. Keane (2012). “Inflammasome proteins in cerebrospinal fluid of brain-injured patients as biomarkers of functional outcome: clinical article.” J Neurosurg 117(6): 1119-1125.
Ahmad, F., N. Mishra, G. Ahrenstorf, B. S. Franklin, E. Latz, R. E. Schmidt and L. Bossaller (2018). “Evidence of inflammasome activation and formation of monocyte-derived ASC specks in HIV-1 positive patients.” AIDS 32(3): 299-307.
Baroja-Mazo, A., F. Martin-Sanchez, A. I. Gomez, C. M. Martinez, J. Amores-Iniesta, V. Compan, M. Barbera-Cremades, J. Yague, E. Ruiz-Ortiz, J. Anton, S. Bujan, I. Couillin, D. Brough, J. I. Arostegui and P. Pelegrin (2014). “The NLRP3 inflammasome is released as a particulate danger signal that amplifies the inflammatory response.” Nat Immunol 15(8): 738-748.
Basiorka, A. A., K. L. McGraw, F. Abbas-Aghababazadeh, A. F. McLemore, N. D. Vincelette, G. A. Ward, E. A. Eksioglu, D. A. Sallman, N. A. Ali, E. Padron, J. Pinilla-Ibarz, R. Komrokji, E. Masala, V. Santini, O. Kosmider, M. Fontenay, P. Fenaux, L. Sokol, S. Wei, B. Fridley and A. F. List (2018). “Assessment of ASC specks as a putative biomarker of pyroptosis in myelodysplastic syndromes: an observational cohort study.” Lancet Haematol 5(9): e393-e402.
Bohlen C J, Bennett F C, Tucker A F, Collins H Y, Mulinyawe S B and Barres B A (2017). “Diverse Requirements for Microglial Survival, Specification, and Function Revealed by Defined-Medium Cultures.” Neuron 94(4): 759-773.
Broz, P. and V. M. Dixit (2016). “Inflammasomes: mechanism of assembly, regulation and signalling.” Nat Rev Immunol 16(7): 407-420.
Cyr, B., R. W. Keane and J. P. de Rivero Vaccari (2020). “ASC, IL-18 and Galectin-3 as Biomarkers of Non-Alcoholic Steatohepatitis: A Proof of Concept Study.” Int J Mol Sci 21(22).
de Rivero Vaccari, J. P., G. Lotocki, O. F. Alonso, H. M. Bramlett, W. D. Dietrich and R. W. Keane (2009). Therapeutic neutralization of the NLRP1 inflammasome reduces the innate immune response and improves histopathology after traumatic brain injury.” J Cereb Blood Flow Metab 29(7): 1251-1261.
de Rivero Vaccari, J. P., G. Lotocki, A. E. Marcillo, W. D. Dietrich and R. W. Keane (2008). “A molecular platform in neurons regulates inflammation after spinal cord injury.” J Neurosci 28(13): 3404-3414.
Desu, H. L., M. Plastini, P. Illiano, H. M. Bramlett, W. D. Dietrich, J. P. de Rivero Vaccari, R. Brambilla and R. W. Keane (2020). “IC100: a novel anti-ASC monoclonal antibody improves functional outcomes in an animal model of multiple sclerosis.” J Neuroinflammation 17(1): 143.
Dunbar J, Krawczyk K, Leem J, Marks C, Nowak J, Regep C, Georges G, Kelm S, Popovic B, Deane C M. (2016) “SAbPred: a structure-based antibody prediction server.” Nucleic Acids Res. Jul 8; 44(W1):W474-8.
Ehrenmann F., Kaas Q. and Lefranc M.-P. (2010). “IMGT/3Dstructure-DB anIMGT/DomainGapAlign: a database and a tool for immunoglobulins or antibodies, T cell receptors, MHC, IgSF and MhcSF” Nucleic Acids Res., 38:D301-D307
Fernandes-Alnemri, T., J. Wu, J. W. Yu, P. Datta, B. Miller, W. Jankowski, S. Rosenberg, J. Zhang and E. S. Alnemri (2007). “The pyroptosome: a supramolecular assembly of ASC dimers mediating inflammatory cell death via caspase-1 activation.” Cell Death Differ 14(9): 1590-1604.
Forouzandeh, M., J. Besen, R. W. Keane and J. P. de Rivero Vaccari (2020). “The Inflammasome Signaling Proteins ASC and IL-18 as Biomarkers of Psoriasis.” Front Pharmacol 11: 1238.
Franklin, B. S., L. Bossaller, D. De Nardo, J. M. Ratter, A. Stutz, G. Engels, C. Brenker, M. Nordhoff, S. R. Mirandola, A. Al-Amoudi, M. S. Mangan, S. Zimmer, B. G. Monks, M. Fricke, R. E. Schmidt, T. Espevik, B. Jones, A. G. Jarnicki, P. M. Hansbro, P. Busto, A. Marshak-Rothstein, S. Hornemann, A. Aguzzi, W. Kastenmuller and E. Latz (2014). “The adaptor ASC has extracellular and ‘prionoid’ activities that propagate inflammation.” Nat Immunol 15(8): 727-737.
Franklin, B. S., E. Latz and F. I. Schmidt (2018). “The intra- and extracellular functions of ASC specks.” Immunol Rev 281(1): 74-87.
Gong, Q., K. Robinson, C. Xu, P. T. Huynh, K. H. C. Chong, E. Y. J. Tan, J. Zhang, Z. Z. Boo, D. E. T. Teo, K. Lay, Y. Zhang, J. S. Y. Lim, W. I. Goh, G. Wright, F. L. Zhong, B. Reversade and B. Wu (2021). “Structural basis for distinct inflammasome complex assembly by human NLRP1 and CARD8.” Nat Commun 12(1): 188.
Guo, H., J. B. Callaway and J. P. Ting (2015). “Inflammasomes: mechanism of action, role in disease, and therapeutics.” Nat Med 21(7): 677-687.
Keane, R. W., W. D. Dietrich and J. P. de Rivero Vaccari (2018). “Inflammasome Proteins As Biomarkers of Multiple Sclerosis.” Front Neurol 9: 135.
Kerr, N., M. Garcia-Contreras, S. Abbassi, N. H. Mejias, B. R. Desousa, C. Ricordi, W. D. Dietrich, R. W. Keane and J. P. de Rivero Vaccari (2018). “Inflammasome Proteins in Serum and Serum-Derived Extracellular Vesicles as Biomarkers of Stroke.” Front Mol Neurosci 11: 309.
Lee, S., A. Ishitsuka, T. Kuroki, Y. H. Lin, A. Shibuya, T. Hongu, Y. Funakoshi, Y. Kanaho, K. Nagata and A. Kawaguchi (2021). “Arf6 exacerbates allergic asthma through cell-to-cell transmission of ASC inflammasomes.” JCI Insight 6(16).
Paramel Varghese, G., L. Folkersen, R. J. Strawbridge, B. Halvorsen, A. Yndestad, T. Ranheim, K. Krohg-Sorensen, M. Skjelland, T. Espevik, P. Aukrust, M. Lengquist, U. Hedin, J. H. Jansson, K. Fransen, G. K. Hansson, P. Eriksson and A. Sirsjo (2016). “NLRP3 Inflammasome Expression and Activation in Human Atherosclerosis.” J Am Heart Assoc 5(5).
Protti, M. P. and L. De Monte (2020). “Dual Role of Inflammasome Adaptor ASC in Cancer.” Front Cell Dev Biol 8: 40.
Rodrigues, T. S., K. S. G. de Sa, A. Y. Ishimoto, A. Becerra, S. Oliveira, L. Almeida, A. V. Goncalves, D. B. Perucello, W. A. Andrade, R. Castro, F. P. Veras, J. E. Toller-Kawahisa, D. C. Nascimento, M. H. F. de Lima, C. M. S. Silva, D. B. Caetite, R. B. Martins, I. A. Castro, M. C. Pontelli, F. C. de Barros, N. B. do Amaral, M. C. Giannini, L. P. Bonjorno, M. I. F. Lopes, R. C. Santana, F. C. Vilar, M. Auxiliadora-Martins, R. Luppino-Assad, S. C. L. de Almeida, F. R. de Oliveira, S. S. Batah, L. Siyuan, M. N. Benatti, T. M. Cunha, J. C. Alves-Filho, F. Q. Cunha, L. D. Cunha, F. G. Frantz, T. Kohlsdorf, A. T. Fabro, E. Arruda, R. D. R. de Oliveira, P. Louzada-Junior and D. S. Zamboni (2021). “Inflammasomes are activated in response to SARS-CoV-2 infection and are associated with COVID-19 severity in patients.” J Exp Med 218(3).
Rowczenio, D. M., S. Pathak, J. I. Arostegui, A. Mensa-Vilaro, E. Omoyinmi, P. Brogan, D. Lipsker, T. Scambler, R. Owen, H. Trojer, A. Baginska, J. D. Gillmore, A. D. Wechalekar, T. Lane, R. Williams, T. Youngstein, P. N. Hawkins, S. Savic and H. J. Lachmann (2018). “Molecular genetic investigation, clinical features, and response to treatment in 21 patients with Schnitzler syndrome.” Blood 131(9): 974-981.
Sborgi, L., Ude, J., Dick, M. S., Vesin, J., Chambon, M., Turcatti, G., Broz, P., S. Hiller (2018). “Assay for high-throughput screening of inhibitors of the ASC-PYD inflammasome core filament.” Cell Stress Apr; 2(4): 82-90.
Scambler, T., H. H. Jarosz-Griffiths, S. Lara-Reyna, S. Pathak, C. Wong, J. Holbrook, F. Martinon, S. Savic, D. Peckham and M. F. McDermott (2019). “ENaC-mediated sodium influx exacerbates NLRP3-dependent inflammation in cystic fibrosis.” Elife 8.
Sharma, D. and T. D. Kanneganti (2016). “The cell biology of inflammasomes: Mechanisms of inflammasome activation and regulation.” J Cell Biol 213(6): 617-629.
Toldo, S., R. Bussani, V. Nuzzi, A. Bonaventura, A. G. Mauro, A. Cannata, R. Pillappa, G. Sinagra, P. Nana-Sinkam, P. Sime and A. Abbate (2021). “Inflammasome formation in the lungs of patients with fatal COVID-19.” Inflamm Res 70(1): 7-10.
Venegas, C., S. Kumar, B. S. Franklin, T. Dierkes, R. Brinkschulte, D. Tejera, A. Vieira-Saecker, S. Schwartz, F. Santarelli, M. P. Kummer, A. Griep, E. Gelpi, M. Beilharz, D. Riedel, D. T. Golenbock, M. Geyer, J. Walter, E. Latz and M. T. Heneka (2017). “Microglia-derived ASC specks cross-seed amyloid-beta in Alzheimer's disease.” Nature 552(7685): 355-361.
Xie, W. H., J. Ding, X. X. Xie, X. H. Yang, X. F. Wu, Z. X. Chen, Q. L. Guo, W. Y. Gao, X. Z. Wang and D. Li (2020). “Hepatitis B virus X protein promotes liver cell pyroptosis under oxidative stress through NLRP3 inflammasome activation.” Inflamm Res 69(7): 683-696.
Ye J, Ma N, Madden T L, Ostell J M. (2013) “IgBLAST: an immunoglobulin variable domain sequence analysis tool.” Nucleic Acids Res. Jul; 41(Web Server issue):W34-40.
Varghese et al., NLRP3 Inflammasome Expression and Activation in Human Atherosclerosis, doi: 10.1161/JAHA.115.003031

1.-82. (canceled)

83. An ASC binding molecule that binds an ASC speck or non-polymerized ASC.

84. The ASC binding molecule of claim 83, that binds:
a) preferentially ASC specks over non-polymerized ASC; or
b) preferentially non-polymerized ASC over ASC specks; or
c) ASC specks and does not bind to non-polymerized ASC; or
d) non-polymerized ASC and does not bind to ASC specks.

85. The ASC binding molecule of claim 83, that prevents or inhibits ASC polymerization, optionally wherein the ASC polymerization is measured in vitro, preferably by an ASC polymerization assay.

86. The ASC binding molecule of claim 83, that prevents or inhibits propagation of ASC-dependent inflammation, optionally wherein:
a) the propagation of inflammation is measured in vitro or in vivo; or
b) the prevention or inhibition of propagation of inflammation is prevention or inhibition of IL-1β release, optionally wherein IL-1β release is measured in vitro, preferably in an assay employing phagocytic cells such as macrophages or microglia.

87. The ASC binding molecule of claim 83, wherein the ASC binding molecule:
a) increases the uptake of ASC extracellular specks by phagocytic cells such as macrophages or microglia; or
b) prevents or inhibits accumulation of ASC and/or ASC specks, optionally wherein the ASC or ASC speck accumulation is intracellular or extracellular.

88. The ASC binding molecule of claim 83 that:
a) prevents, reduces, or inhibits demyelination, optionally wherein prevention, reduction, or inhibition of demyelination is improving demyelination score in vivo; or
b) reduces levels of reactive microglia in vivo; or
c) reduces levels of ASC and/or cleaved caspase-1 protein in vivo; or
d) reduces levels of infiltrating CD4+ T-cells in the spinal cord in vivo.

89. The ASC binding molecule of claim 83, which binds to an epitope of:
a) human ASC of SEQ ID NO:1; or
b) mouse ASC of SEQ ID NO: 2.

90. The ASC binding molecule of claim 89, wherein the epitope is in the ASC PYD domain or ASC CARD domain.

91. The ASC binding molecule of claim 83, which binds to an epitope, either:
a) in the ASC CARD domain comprising, consisting essentially of, or consisting of amino acid residues numbered:
i. 174 and 175;
ii. 115, 116, 119, 120, 174, 175, 184, 186 and 187;
iii. 115, 116, 170, 171, 172, 174, 175, 186 and 187;
iv. 137;
v. 119, 120, 178, 179, 186 and 187; or
vi. 119, 120, 174, 175, 186 and 187;
optionally wherein the epitope in the ASC CARD domain comprises, consists essentially of, or consists of amino acid residues:
i. K174 and D175;
ii. 1115, D116, R119, A120, K174, D175, S184, Q185, S186 and Y187;
iii. 1115, D116, N170, W171, T172, K174, D175, S186 and Y187;
iv. Y137;
v. R119, A120, L178, Q179, S186 and Y187; or
vi. R119, A120, K174, D175, S186 and Y187;
optionally wherein the amino acid residues are with respect to human ASC (SEQ ID NO: 1) or;
b) in the ASC PYD domain comprising, consisting essentially of, or consisting of amino acid residues numbered:
i. 9, 10, 13, 14, 18 and 19;
ii. 9, 10, 13 and 14;
iii. 13 and 14;
iv. 79, 80, 83 and 84; or
v. 18, 19, 30, 31, 71, 74, 75, 77, 79 and 80;
optionally wherein the epitope in the ASC PYD domain comprises, consists essentially of, or consists of amino acid residues:
i. L9, D10, E13, N14, E18 and E19;
ii. L9, D10, E13 and N14;
iii. E13 and N14;
iv. Q79, E80, G83 and Q84; or
v. E18, E19, V30, P31, N71, R74, D75, G77, Q79 and E80;
optionally wherein the amino acid residues are with respect to human ASC (SEQ ID NO: 1).

92. The ASC binding molecule of claim 83, comprising:
a. a VH-CDR1 comprising the amino acid sequence of SEQ ID NO: 201, a VH-CDR2 comprising the amino acid sequence of SEQ ID NO: 202; a VH-CDR3 comprising the amino acid sequence SEQ ID NO: 203, a VL-CDR1 comprising the amino sequence SEQ ID NO: 205; a VL-CDR2 comprising the amino sequence SEQ ID NO: 206 and a VL-CDR3 comprising the amino sequence SEQ ID NO: 207; or
b. a VH-CDR1 comprising the amino acid sequence of SEQ ID NO: 201, a VH-CDR2 comprising the amino acid sequence of SEQ ID NO: 412, a VH-CDR3 comprising the amino acid sequence SEQ ID NO: 203, a VL-CDR1 comprising the amino sequence SEQ ID NO: 205, a VL-CDR2 comprising the amino sequence SEQ ID NO: 206 and a VL-CDR3 comprising the amino sequence SEQ ID NO: 207; or
c. a VH-CDR1 comprising the amino acid sequence of SEQ ID NO: 201, a VH-CDR2 comprising the amino acid sequence of SEQ ID NO: 412, a VH-CDR3 comprising the amino acid sequence SEQ ID NO: 203, a VL-CDR1 comprising the amino sequence SEQ ID NO: 435, a VL-CDR2 comprising the amino sequence SEQ ID NO: 206 and a VL-CDR3 comprising the amino sequence SEQ ID NO: 207; or
d. a VH-CDR1 comprising the amino acid sequence of SEQ ID NO: 201, a VH-CDR2 comprising the amino acid sequence of SEQ ID NO: 462, a VH-CDR3 comprising the amino acid sequence SEQ ID NO: 203, a VL-CDR1 comprising the amino sequence SEQ ID NO: 205, a VL-CDR2 comprising the amino sequence SEQ ID NO: 206 and a VL-CDR3 comprising the amino sequence SEQ ID NO: 207; or
e. a VH-CDR1 comprising the amino acid sequence of SEQ ID NO: 201, a VH-CDR2 comprising the amino acid sequence of SEQ ID NO: 462, a VH-CDR3 comprising the amino acid sequence SEQ ID NO: 203, a VL-CDR1 comprising the amino sequence SEQ ID NO: 435, a VL-CDR2 comprising the amino sequence SEQ ID NO: 206 and a VL-CDR3 comprising the amino sequence SEQ ID NO: 207; or
f. a VH-CDR1 comprising the amino acid sequence of SEQ ID NO: 201, a VH-CDR2 comprising the amino acid sequence of SEQ ID NO: 472, a VH-CDR3 comprising the amino acid sequence SEQ ID NO: 203, a VL-CDR1 comprising the amino sequence SEQ ID NO: 205, a VL-CDR2 comprising the amino sequence SEQ ID NO: 206 and a VL-CDR3 comprising the amino sequence SEQ ID NO: 207; or
g. a VH-CDR1 comprising the amino acid sequence of SEQ ID NO: 201, a VH-CDR2 comprising the amino acid sequence of SEQ ID NO: 472, a VH-CDR3 comprising the amino acid sequence SEQ ID NO: 203, a VL-CDR1 comprising the amino sequence SEQ ID NO: 435, a VL-CDR2 comprising the amino sequence SEQ ID NO: 206 and a VL-CDR3 comprising the amino sequence SEQ ID NO: 207.

93. The ASC binding molecule of claim 83, comprising:
a. a VH-CDR1 comprising the amino acid sequence of SEQ ID NO: 201, a VH-CDR2 comprising the amino acid sequence of SEQ ID NO: 202 and a VH-CDR3 comprising the amino acid sequence of SEQ ID NO: 203; or
b. a VH-CDR1 comprising the amino acid sequence of SEQ ID NO: 201, a VH-CDR2 comprising the amino acid sequence of SEQ ID NO: 412 and a VH-CDR3 comprising the amino acid sequence of SEQ ID NO: 203; or
c. a VH-CDR1 comprising the amino acid sequence of SEQ ID NO: 201, a VH-CDR2 comprising the amino acid sequence of SEQ ID NO: 462 and a VH-CDR3 comprising the amino acid sequence of SEQ ID NO: 203; or
d. a VH-CDR1 comprising the amino acid sequence of SEQ ID NO: 201, a VH-CDR2 comprising the amino acid sequence of SEQ ID NO: 472 and a VH-CDR3 comprising the amino acid sequence SEQ ID NO: 203; or
a. a VL-CDR1 comprising the amino acid sequence of SEQ ID NO: 205, a VL-CDR2 comprising the amino acid sequence of SEQ ID NO: 206, a VL-CDR3 comprising the amino acid sequence SEQ ID NO: 207; or
b. a VL-CDR1 comprising the amino acid sequence of SEQ ID NO: 435, a VL-CDR2 comprising the amino acid sequence of SEQ ID NO: 206, a VL-CDR3 comprising the amino acid sequence SEQ ID NO: 207.

94. The ASC binding molecule of claim 83, comprising:
a. a Heavy Chain Variable Region (VH) comprising the amino acid sequence of SEQ ID NO: 400, or having at least 93%, 94%, 95%, 96%, 97%, 98% or 99% sequence identity to the amino acid sequence of SEQ ID NO: 400, and a Light Chain Variable Region (VL) comprising the amino acid sequence of SEQ ID NO: 404 or a Light Chain Variable Region (VL) having at least 95%, 96%, 97%, 98% or 99% sequence identity to the amino acid sequence of SEQ ID NO: 404; or
b. a Heavy Chain Variable Region (VH) comprising the amino acid sequence of SEQ ID NO: 410, or having at least 93%, 94%, 95%, 96%, 97%, 98% or 99% sequence identity to the amino acid sequence of SEQ ID NO: 410, and a Light Chain Variable Region (VL) comprising the amino acid sequence of SEQ ID NO: 414 or a Light Chain Variable Region (VL) having at least 95%, 96%, 97%, 98% or 99% sequence identity to the amino acid sequence of SEQ ID NO: 414; or
c. a Heavy Chain Variable Region (VH) comprising the amino acid sequence of SEQ ID NO: 420, or having at least 93%, 94%, 95%, 96%, 97%, 98% or 99% sequence identity to the amino acid sequence of SEQ ID NO: 420, and a Light Chain Variable Region (VL) comprising the amino acid sequence of SEQ ID NO: 424 or a Light Chain Variable Region (VL) having at least 95%, 96%, 97%, 98% or 99% sequence identity to the amino acid sequence of SEQ ID NO: 424; or
d. a Heavy Chain Variable Region (VH) comprising the amino acid sequence of SEQ ID NO: 430, or having at least 93%, 94%, 95%, 96%, 97%, 98% or 99% sequence identity to the amino acid sequence of SEQ ID NO: 430, and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
e. a Heavy Chain Variable Region (VH) comprising the amino acid sequence of SEQ ID NO: 440, or having at least 93%, 94%, 95%, 96%, 97%, 98% or 99% sequence identity to the amino acid sequence of SEQ ID NO: 440, and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
f. a Heavy Chain Variable Region (VH) comprising the amino acid sequence of SEQ ID NO: 450, or having at least 93%, 94%, 95%, 96%, 97%, 98% or 99% sequence identity to the amino acid sequence of SEQ ID NO: 450, and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
g. a Heavy Chain Variable Region (VH) comprising the amino acid sequence of SEQ ID NO: 460, or having at least 93%, 94%, 95%, 96%, 97%, 98% or 99% sequence identity to the amino acid sequence of SEQ ID NO: 460, and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
h. a Heavy Chain Variable Region (VH) comprising the amino acid sequence of SEQ ID NO: 470, or having at least 93%, 94%, 95%, 96%, 97%, 98% or 99% sequence identity to the amino acid sequence of SEQ ID NO: 470, and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
i. a Heavy Chain Variable Region (VH) comprising the amino acid sequence of SEQ ID NO: 480, or having at least 93%, 94%, 95%, 96%, 97%, 98% or 99% sequence identity to the amino acid sequence of SEQ ID NO: 480, and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
j. a Heavy Chain Variable Region (VH) comprising the amino acid sequence of SEQ ID NO: 490, or having at least 93%, 94%, 95%, 96%, 97%, 98% or 99% sequence identity to the amino acid sequence of SEQ ID NO: 490, and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
k. a Heavy Chain Variable Region (VH) comprising the amino acid sequence of SEQ ID NO: 500, or having at least 93%, 94%, 95%, 96%, 97%, 98% or 99% sequence identity to the amino acid sequence of SEQ ID NO: 500, and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
l. a Heavy Chain Variable Region (VH) comprising the amino acid sequence of SEQ ID NO: 510, or having at least 93%, 94%, 95%, 96%, 97%, 98% or 99% sequence identity to the amino acid sequence of SEQ ID NO: 510, and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
m. a Heavy Chain Variable Region (VH) comprising the amino acid sequence of SEQ ID NO: 520, or having at least 93%, 94%, 95%, 96%, 97%, 98% or 99% sequence identity to the amino acid sequence of SEQ ID NO: 520, and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
n. a Heavy Chain Variable Region (VH) comprising the amino acid sequence of SEQ ID NO: 530, or having at least 93%, 94%, 95%, 96%, 97%, 98% or 99% sequence identity to the amino acid sequence of SEQ ID NO: 530, and a Light Chain Variable Region (VL) comprising the amino acid sequence of SEQ ID NO: 424 or a Light Chain Variable Region (VL) having at least 95%, 96%, 97%, 98% or 99% sequence identity to the amino acid sequence of SEQ ID NO: 424; or
o. a Heavy Chain Variable Region (VH) comprising the amino acid sequence of SEQ ID NO: 540, or having at least 93%, 94%, 95%, 96%, 97%, 98% or 99% sequence identity to the amino acid sequence of SEQ ID NO: 540, and a Light Chain Variable Region (VL) comprising the amino acid sequence of SEQ ID NO: 424 or a Light Chain Variable Region (VL) having at least 95%, 96%, 97%, 98% or 99% sequence identity to the amino acid sequence of SEQ ID NO: 424; or
p. a Heavy Chain Variable Region (VH) comprising the amino acid sequence of SEQ ID NO: 400, or having at least 93%, 94%, 95%, 96%, 97%, 98% or 99% sequence identity to the amino acid sequence of SEQ ID NO: 400, and a Light Chain Variable Region (VL) comprising the amino acid sequence of SEQ ID NO: 414 or a Light Chain Variable Region (VL) having at least 95%, 96%, 97%, 98% or 99% sequence identity to the amino acid sequence of SEQ ID NO: 414; or
q. a Heavy Chain Variable Region (VH) comprising the amino acid sequence of SEQ ID NO: 410, or having at least 93%, 94%, 95%, 96%, 97%, 98% or 99% sequence identity to the amino acid sequence of SEQ ID NO: 410, and a Light Chain Variable Region (VL) comprising the amino acid sequence of SEQ ID NO: 404 or a Light Chain Variable Region (VL) having at least 95%, 96%, 97%, 98% or 99% sequence identity to the amino acid sequence of SEQ ID NO: 404; or
r. a Heavy Chain Variable Region (VH) comprising the amino acid sequence of SEQ ID NO: 410, or having at least 93%, 94%, 95%, 96%, 97%, 98% or 99% sequence identity to the amino acid sequence of SEQ ID NO: 410, and a Light Chain Variable Region (VL) comprising the amino acid sequence of SEQ ID NO: 424 or a Light Chain Variable Region (VL) having at least 95%, 96%, 97%, 98% or 99% sequence identity to the amino acid sequence of SEQ ID NO: 424; or
s. a Heavy Chain Variable Region (VH) comprising the amino acid sequence of SEQ ID NO: 410, or having at least 93%, 94%, 95%, 96%, 97%, 98% or 99% sequence identity to the amino acid sequence of SEQ ID NO: 410, and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
t. a Heavy Chain Variable Region (VH) comprising the amino acid sequence of SEQ ID NO: 420, or having at least 93%, 94%, 95%, 96%, 97%, 98% or 99% sequence identity to the amino acid sequence of SEQ ID NO: 420, and a Light Chain Variable Region (VL) comprising the amino acid sequence of SEQ ID NO: 404 or a Light Chain Variable Region (VL) having at least 95%, 96%, 97%, 98% or 99% sequence identity to the amino acid sequence of SEQ ID NO: 404; or
u. a Heavy Chain Variable Region (VH) comprising the amino acid sequence of SEQ ID NO: 420, or having at least 93%, 94%, 95%, 96%, 97%, 98% or 99% sequence identity to the amino acid sequence of SEQ ID NO: 420, and a Light Chain Variable Region (VL) comprising the amino acid sequence of SEQ ID NO: 414 or a Light Chain Variable Region (VL) having at least 95%, 96%, 97%, 98% or 99% sequence identity to the amino acid sequence of SEQ ID NO: 414; or
v. a Heavy Chain Variable Region (VH) comprising the amino acid sequence of SEQ ID NO: 430, or having at least 93%, 94%, 95%, 96%, 97%, 98% or 99% sequence identity to the amino acid sequence of SEQ ID NO: 430, and a Light Chain Variable Region (VL) comprising the amino acid sequence of SEQ ID NO: 404 or a Light Chain Variable Region (VL) having at least 95%, 96%, 97%, 98% or 99% sequence identity to the amino acid sequence of SEQ ID NO: 404; or
w. a Heavy Chain Variable Region (VH) comprising the amino acid sequence of SEQ ID NO: 430, or having at least 93%, 94%, 95%, 96%, 97%, 98% or 99% sequence identity to the amino acid sequence of SEQ ID NO: 430, and a Light Chain Variable Region (VL) comprising the amino acid sequence of SEQ ID NO: 414 or a Light Chain Variable Region (VL) having at least 95%, 96%, 97%, 98% or 99% sequence identity to the amino acid sequence of SEQ ID NO: 414; or
x. a Heavy Chain Variable Region (VH) comprising the amino acid sequence of SEQ ID NO: 400, or having at least 93%, 94%, 95%, 96%, 97%, 98% or 99% sequence identity to the amino acid sequence of SEQ ID NO: 400, and a Light Chain Variable Region (VL) comprising the amino acid sequence of SEQ ID NO: 424 or a Light Chain Variable Region (VL) having at least 95%, 96%, 97%, 98% or 99% sequence identity to the amino acid sequence of SEQ ID NO: 424; or
y. a Heavy Chain Variable Region (VH) comprising the amino acid sequence of SEQ ID NO: 450, or having at least 93%, 94%, 95%, 96%, 97%, 98% or 99% sequence identity to the amino acid sequence of SEQ ID NO: 450, and a Light Chain Variable Region (VL) comprising the amino acid sequence of SEQ ID NO: 424 or a Light Chain Variable Region (VL) having at least 95%, 96%, 97%, 98% or 99% sequence identity to the amino acid sequence of SEQ ID NO: 424; or
z. a Heavy Chain Variable Region (VH) comprising the amino acid sequence of SEQ ID NO: 480, or having at least 93%, 94%, 95%, 96%, 97%, 98% or 99% sequence identity to the amino acid sequence of SEQ ID NO: 480, and a Light Chain Variable Region (VL) comprising the amino acid sequence of SEQ ID NO: 424 or a Light Chain Variable Region (VL) having at least 95%, 96%, 97%, 98% or 99% sequence identity to the amino acid sequence of SEQ ID NO: 424; or
aa. a Heavy Chain Variable Region (VH) comprising the amino acid sequence of SEQ ID NO: 520, or having at least 93%, 94%, 95%, 96%, 97%, 98% or 99% sequence identity to the amino acid sequence of SEQ ID NO: 520, and a Light Chain Variable Region (VL) comprising the amino acid sequence of SEQ ID NO: 424 or a Light Chain Variable Region (VL) having at least 95%, 96%, 97%, 98% or 99% sequence identity to the amino acid sequence of SEQ ID NO: 424; or
bb. a Heavy Chain Variable Region (VH) comprising the amino acid sequence of SEQ ID NO: 430, or having at least 93%, 94%, 95%, 96%, 97%, 98% or 99% sequence identity to the amino acid sequence of SEQ ID NO: 430, and a Light Chain Variable Region (VL) comprising the amino acid sequence of SEQ ID NO: 424 or a Light Chain Variable Region (VL) having at least 95%, 96%, 97%, 98% or 99% sequence identity to the amino acid sequence of SEQ ID NO: 424.

95. The ASC binding molecule of claim 83, comprising:
a. a Heavy Chain Variable Region (VH) comprising the amino acid sequence of SEQ ID NO: 400, or having at least 93%, 94%, 95%, 96%, 97%, 98% or 99% sequence identity to the amino acid sequence of SEQ ID NO: 400, and a Light Chain Variable Region (VL) comprising the amino acid sequence of SEQ ID NO: 404; or
b. a Heavy Chain Variable Region (VH) comprising the amino acid sequence of SEQ ID NO: 410, or having at least 93%, 94%, 95%, 96%, 97%, 98% or 99% sequence identity to the amino acid sequence of SEQ ID NO: 410, and a Light Chain Variable Region (VL) comprising the amino acid sequence of SEQ ID NO: 414; or
c. a Heavy Chain Variable Region (VH) comprising the amino acid sequence of SEQ ID NO: 420, or having at least 93%, 94%, 95%, 96%, 97%, 98% or 99% sequence identity to the amino acid sequence of SEQ ID NO: 420, and a Light Chain Variable Region (VL) comprising the amino acid sequence of SEQ ID NO: 424; or
d. a Heavy Chain Variable Region (VH) comprising the amino acid sequence of SEQ ID NO: 430, or having at least 93%, 94%, 95%, 96%, 97%, 98% or 99% sequence identity to the amino acid sequence of SEQ ID NO: 430, and a Light Chain Variable Region (VL) comprising the amino acid sequence of SEQ ID NO: 434; or
e. a Heavy Chain Variable Region (VH) comprising the amino acid sequence of SEQ ID NO: 440, or having at least 93%, 94%, 95%, 96%, 97%, 98% or 99% sequence identity to the amino acid sequence of SEQ ID NO: 440, and a Light Chain Variable Region (VL) comprising the amino acid sequence of SEQ ID NO: 434; or
f. a Heavy Chain Variable Region (VH) comprising the amino acid sequence of SEQ ID NO: 450, or having at least 93%, 94%, 95%, 96%, 97%, 98% or 99% sequence identity to the amino acid sequence of SEQ ID NO: 450, and a Light Chain Variable Region (VL) comprising the amino acid sequence of SEQ ID NO: 434; or
g. a Heavy Chain Variable Region (VH) comprising the amino acid sequence of SEQ ID NO: 460, or having at least 93%, 94%, 95%, 96%, 97%, 98% or 99% sequence identity to the amino acid sequence of SEQ ID NO: 460, and a Light Chain Variable Region (VL) comprising the amino acid sequence of SEQ ID NO: 434; or
h. a Heavy Chain Variable Region (VH) comprising the amino acid sequence of SEQ ID NO: 470, or having at least 93%, 94%, 95%, 96%, 97%, 98% or 99% sequence identity to the amino acid sequence of SEQ ID NO: 470, and a Light Chain Variable Region (VL) comprising the amino acid sequence of SEQ ID NO: 434; or
i. a Heavy Chain Variable Region (VH) comprising the amino acid sequence of SEQ ID NO: 480, or having at least 93%, 94%, 95%, 96%, 97%, 98% or 99% sequence identity to the amino acid sequence of SEQ ID NO: 480, and a Light Chain Variable Region (VL) comprising the amino acid sequence of SEQ ID NO: 434; or
j. a Heavy Chain Variable Region (VH) comprising the amino acid sequence of SEQ ID NO: 490, or having at least 93%, 94%, 95%, 96%, 97%, 98% or 99% sequence identity to the amino acid sequence of SEQ ID NO: 490, and a Light Chain Variable Region (VL) comprising the amino acid sequence of SEQ ID NO: 434; or
k. a Heavy Chain Variable Region (VH) comprising the amino acid sequence of SEQ ID NO: 500, or having at least 93%, 94%, 95%, 96%, 97%, 98% or 99% sequence identity to the amino acid sequence of SEQ ID NO: 500, and a Light Chain Variable Region (VL) comprising the amino acid sequence of SEQ ID NO: 434; or
l. a Heavy Chain Variable Region (VH) comprising the amino acid sequence of SEQ ID NO: 510, or having at least 93%, 94%, 95%, 96%, 97%, 98% or 99% sequence identity to the amino acid sequence of SEQ ID NO: 510, and a Light Chain Variable Region (VL) comprising the amino acid sequence of SEQ ID NO: 434; or
m. a Heavy Chain Variable Region (VH) comprising the amino acid sequence of SEQ ID NO: 520, or having at least 93%, 94%, 95%, 96%, 97%, 98% or 99% sequence identity to the amino acid sequence of SEQ ID NO: 520, and a Light Chain Variable Region (VL) comprising the amino acid sequence of SEQ ID NO: 434; or
n. a Heavy Chain Variable Region (VH) comprising the amino acid sequence of SEQ ID NO: 530, or having at least 93%, 94%, 95%, 96%, 97%, 98% or 99% sequence identity to the amino acid sequence of SEQ ID NO: 530, and a Light Chain Variable Region (VL) comprising the amino acid sequence of SEQ ID NO: 424; or
o. a Heavy Chain Variable Region (VH) comprising the amino acid sequence of SEQ ID NO: 540, or having at least 93%, 94%, 95%, 96%, 97%, 98% or 99% sequence identity to the amino acid sequence of SEQ ID NO: 540, and a Light Chain Variable Region (VL) comprising the amino acid sequence of SEQ ID NO: 424; or
p. a Heavy Chain Variable Region (VH) comprising the amino acid sequence of SEQ ID NO: 400, or having at least 93%, 94%, 95%, 96%, 97%, 98% or 99% sequence identity to the amino acid sequence of SEQ ID NO: 400, and a Light Chain Variable Region (VL) comprising the amino acid sequence of SEQ ID NO: 414; or
q. a Heavy Chain Variable Region (VH) comprising the amino acid sequence of SEQ ID NO: 410, or having at least 93%, 94%, 95%, 96%, 97%, 98% or 99% sequence identity to the amino acid sequence of SEQ ID NO: 410, and a Light Chain Variable Region (VL) comprising the amino acid sequence of SEQ ID NO: 404; or
r. a Heavy Chain Variable Region (VH) comprising the amino acid sequence of SEQ ID NO: 410, or having at least 93%, 94%, 95%, 96%, 97%, 98% or 99% sequence identity to the amino acid sequence of SEQ ID NO: 410, and a Light Chain Variable Region (VL) comprising the amino acid sequence of SEQ ID NO: 424; or
s. a Heavy Chain Variable Region (VH) comprising the amino acid sequence of SEQ ID NO: 410, or having at least 93%, 94%, 95%, 96%, 97%, 98% or 99% sequence identity to the amino acid sequence of SEQ ID NO: 410, and a Light Chain Variable Region (VL) comprising the amino acid sequence of SEQ ID NO: 434; or
t. a Heavy Chain Variable Region (VH) comprising the amino acid sequence of SEQ ID NO: 420, or having at least 93%, 94%, 95%, 96%, 97%, 98% or 99% sequence identity to the amino acid sequence of SEQ ID NO: 420, and a Light Chain Variable Region (VL) comprising the amino acid sequence of SEQ ID NO: 404; or
u. a Heavy Chain Variable Region (VH) comprising the amino acid sequence of SEQ ID NO: 420, or having at least 93%, 94%, 95%, 96%, 97%, 98% or 99% sequence identity to the amino acid sequence of SEQ ID NO: 420, and a Light Chain Variable Region (VL) comprising the amino acid sequence of SEQ ID NO: 414; or
v. a Heavy Chain Variable Region (VH) comprising the amino acid sequence of SEQ ID NO: 430, or having at least 93%, 94%, 95%, 96%, 97%, 98% or 99% sequence identity to the amino acid sequence of SEQ ID NO: 430, and a Light Chain Variable Region (VL) comprising the amino acid sequence of SEQ ID NO: 404; or
w. a Heavy Chain Variable Region (VH) comprising the amino acid sequence of SEQ ID NO: 430, or having at least 93%, 94%, 95%, 96%, 97%, 98% or 99% sequence identity to the amino acid sequence of SEQ ID NO: 430, and a Light Chain Variable Region (VL) comprising the amino acid sequence of SEQ ID NO: 414; or
x. a Heavy Chain Variable Region (VH) comprising the amino acid sequence of SEQ ID NO: 400, or having at least 93%, 94%, 95%, 96%, 97%, 98% or 99% sequence identity to the amino acid sequence of SEQ ID NO: 400, and a Light Chain Variable Region (VL) comprising the amino acid sequence of SEQ ID NO: 424; or
y. a Heavy Chain Variable Region (VH) comprising the amino acid sequence of SEQ ID NO: 450, or having at least 93%, 94%, 95%, 96%, 97%, 98% or 99% sequence identity to the amino acid sequence of SEQ ID NO: 450, and a Light Chain Variable Region (VL) comprising the amino acid sequence of SEQ ID NO: 424; or
z. a Heavy Chain Variable Region (VH) comprising the amino acid sequence of SEQ ID NO: 480, or having at least 93%, 94%, 95%, 96%, 97%, 98% or 99% sequence identity to the amino acid sequence of SEQ ID NO: 480, and a Light Chain Variable Region (VL) comprising the amino acid sequence of SEQ ID NO: 424; or
aa. a Heavy Chain Variable Region (VH) comprising the amino acid sequence of SEQ ID NO: 520, or having at least 93%, 94%, 95%, 96%, 97%, 98% or 99% sequence identity to the amino acid sequence of SEQ ID NO: 520, and a Light Chain Variable Region (VL) comprising the amino acid sequence of SEQ ID NO: 424; or
bb. a Heavy Chain Variable Region (VH) comprising the amino acid sequence of SEQ ID NO: 430, or having at least 93%, 94%, 95%, 96%, 97%, 98% or 99% sequence identity to the amino acid sequence of SEQ ID NO: 430, and a Light Chain Variable Region (VL) comprising the amino acid sequence of SEQ ID NO: 424.

96. The ASC binding molecule of claim 83, comprising:
a. a Heavy Chain Variable Region (VH) comprising the amino acid sequence of SEQ ID NO: 400 and a Light Chain Variable Region (VL) comprising the amino acid sequence of SEQ ID NO: 404; or
b. a Heavy Chain Variable Region (VH) comprising the amino acid sequence of SEQ ID NO: 410 and a Light Chain Variable Region (VL) comprising the amino acid sequence of SEQ ID NO: 414; or
c. a Heavy Chain Variable Region (VH) comprising the amino acid sequence of SEQ ID NO: 420 and a Light Chain Variable Region (VL) comprising the amino acid sequence of SEQ ID NO: 424; or
d. a Heavy Chain Variable Region (VH) comprising the amino acid sequence of SEQ ID NO: 430 and a Light Chain Variable Region (VL) comprising the amino acid sequence of SEQ ID NO: 434; or
e. a Heavy Chain Variable Region (VH) comprising the amino acid sequence of SEQ ID NO: 440 and a Light Chain Variable Region (VL) comprising the amino acid sequence of SEQ ID NO: 434; or
f. a Heavy Chain Variable Region (VH) comprising the amino acid sequence of SEQ ID NO: 450 and a Light Chain Variable Region (VL) comprising the amino acid sequence of SEQ ID NO: 434; or
g. a Heavy Chain Variable Region (VH) comprising the amino acid sequence of SEQ ID NO: 460 and a Light Chain Variable Region (VL) comprising the amino acid sequence of SEQ ID NO: 434; or
h. a Heavy Chain Variable Region (VH) comprising the amino acid sequence of SEQ ID NO: 470 and a Light Chain Variable Region (VL) comprising the amino acid sequence of SEQ ID NO: 434; or
i. a Heavy Chain Variable Region (VH) comprising the amino acid sequence of SEQ ID NO: 480 and a Light Chain Variable Region (VL) comprising the amino acid sequence of SEQ ID NO: 434; or
j. a Heavy Chain Variable Region (VH) comprising the amino acid sequence of SEQ ID NO: 490 and a Light Chain Variable Region (VL) comprising the amino acid sequence of SEQ ID NO: 434; or
k. a Heavy Chain Variable Region (VH) comprising the amino acid sequence of SEQ ID NO: 500 and a Light Chain Variable Region (VL) comprising the amino acid sequence of SEQ ID NO: 434; or
l. a Heavy Chain Variable Region (VH) comprising the amino acid sequence of SEQ ID NO: 510 and a Light Chain Variable Region (VL) comprising the amino acid sequence of SEQ ID NO: 434; or
m. a Heavy Chain Variable Region (VH) comprising the amino acid sequence of SEQ ID NO: 520 and a Light Chain Variable Region (VL) comprising the amino acid sequence of SEQ ID NO: 434; or
n. a Heavy Chain Variable Region (VH) comprising the amino acid sequence of SEQ ID NO: 530 and a Light Chain Variable Region (VL) comprising the amino acid sequence of SEQ ID NO: 424; or
o. a Heavy Chain Variable Region (VH) comprising the amino acid sequence of SEQ ID NO: 540 and a Light Chain Variable Region (VL) comprising the amino acid sequence of SEQ ID NO: 424; or
p. a Heavy Chain Variable Region (VH) comprising the amino acid sequence of SEQ ID NO: 400 and a Light Chain Variable Region (VL) comprising the amino acid sequence of SEQ ID NO: 414; or
q. a Heavy Chain Variable Region (VH) comprising the amino acid sequence of SEQ ID NO: 410 and a Light Chain Variable Region (VL) comprising the amino acid sequence of SEQ ID NO: 404; or
r. a Heavy Chain Variable Region (VH) comprising the amino acid sequence of SEQ ID NO: 410 and a Light Chain Variable Region (VL) comprising the amino acid sequence of SEQ ID NO: 424; or
s. a Heavy Chain Variable Region (VH) comprising the amino acid sequence of SEQ ID NO: 410 and a Light Chain Variable Region (VL) comprising the amino acid sequence of SEQ ID NO: 434; or
t. a Heavy Chain Variable Region (VH) comprising the amino acid sequence of SEQ ID NO: 420 and a Light Chain Variable Region (VL) comprising the amino acid sequence of SEQ ID NO: 404; or
u. a Heavy Chain Variable Region (VH) comprising the amino acid sequence of SEQ ID NO: 420 and a Light Chain Variable Region (VL) comprising the amino acid sequence of SEQ ID NO: 414; or
v. a Heavy Chain Variable Region (VH) comprising the amino acid sequence of SEQ ID NO: 430 and a Light Chain Variable Region (VL) comprising the amino acid sequence of SEQ ID NO: 404; or
w. a Heavy Chain Variable Region (VH) comprising the amino acid sequence of SEQ ID NO: 430 and a Light Chain Variable Region (VL) comprising the amino acid sequence of SEQ ID NO: 414; or
x. a Heavy Chain Variable Region (VH) comprising the amino acid sequence of SEQ ID NO: 400 and a Light Chain Variable Region (VL) comprising the amino acid sequence of SEQ ID NO: 424; or
y. a Heavy Chain Variable Region (VH) comprising the amino acid sequence of SEQ ID NO: 450 and a Light Chain Variable Region (VL) comprising the amino acid sequence of SEQ ID NO: 424; or
z. a Heavy Chain Variable Region (VH) comprising the amino acid sequence of SEQ ID NO: 480 and a Light Chain Variable Region (VL) comprising the amino acid sequence of SEQ ID NO: 424; or
aa. a Heavy Chain Variable Region (VH) comprising the amino acid sequence of SEQ ID NO: 520 and a Light Chain Variable Region (VL) comprising the amino acid sequence of SEQ ID NO: 424; or
bb. a Heavy Chain Variable Region (VH) comprising the amino acid sequence of SEQ ID NO: 430 and a Light Chain Variable Region (VL) comprising the amino acid sequence of SEQ ID NO: 424.

97. The ASC binding molecule of claim 83, comprising:
a. a VH-CDR1 comprising the amino acid sequence of SEQ ID NO: 201, a VH-CDR2 comprising the amino acid sequence of SEQ ID NO: 412, a VH-CDR3 comprising the amino acid sequence SEQ ID NO: 203, a VL-CDR1 comprising the amino sequence of SEQ ID NO: 205, a VL-CDR2 comprising the amino sequence of SEQ ID NO: 206, and a VL-CDR3 comprising the amino sequence SEQ ID NO: 207; or
b. a VH-CDR1 comprising the amino acid sequence of SEQ ID NO: 201, a VH-CDR2 comprising the amino acid sequence of SEQ ID NO: 462, a VH-CDR3 comprising the amino acid sequence of SEQ ID NO: 203, a VL-CDR1 comprising the amino sequence of SEQ ID NO: 435, a VL-CDR2 comprising the amino sequence of SEQ ID NO: 206, and a VL-CDR3 comprising the amino sequence of SEQ ID NO: 207; or
c. a VH-CDR1 comprising the amino acid sequence of SEQ ID NO: 201, a VH-CDR2 comprising the amino acid sequence of SEQ ID NO: 412, a VH-CDR3 comprising the amino acid sequence of SEQ ID NO: 203, a VL-CDR1 comprising the amino sequence of SEQ ID NO: 435, a VL-CDR2 comprising the amino sequence of SEQ ID NO: 206, and a VL-CDR3 comprising the amino sequence of SEQ ID NO: 207;
or comprising:
a. a Heavy Chain Variable Region (VH) comprising the amino acid sequence of SEQ ID NO: 440, or having at least 93%, 94%, 95%, 96%, 97%, 98% or 99% sequence identity to the amino acid sequence of SEQ ID NO: 440 and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
b. a Heavy Chain Variable Region (VH) comprising the amino acid sequence of SEQ ID NO: 460, or having at least 93%, 94%, 95%, 96%, 97%, 98% or 99% sequence identity to the amino acid sequence of SEQ ID NO: 460 and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
c. a Heavy Chain Variable Region (VH) comprising the amino acid sequence of SEQ ID NO: 520, or having at least 93%, 94%, 95%, 96%, 97%, 98% or 99% sequence identity to the amino acid sequence of SEQ ID NO: 520 and a Light Chain Variable Region (VL) comprising the amino acid sequence of SEQ ID NO: 434 or a Light Chain Variable Region (VL) having at least 95%, 96%, 97%, 98% or 99% sequence identity to the amino acid sequence of SEQ ID NO: 434; or
d. a Heavy Chain Variable Region (VH) comprising the amino acid sequence of SEQ ID NO: 480, or having at least 93%, 94%, 95%, 96%, 97%, 98% or 99% sequence identity to the amino acid sequence of SEQ ID NO: 480 and a Light Chain Variable Region (VL) comprising the amino acid sequence of SEQ ID NO: 424 or a Light Chain Variable Region (VL) having at least 95%, 96%, 97%, 98% or 99% sequence identity to the amino acid sequence of SEQ ID NO: 424;
or comprising:
a. a Heavy Chain Variable Region (VH) comprising the amino acid sequence of SEQ ID NO: 440, or having at least 93%, 94%, 95%, 96%, 97%, 98% or 99% sequence identity to the amino acid sequence of SEQ ID NO: 440 and a Light Chain Variable Region (VL) comprising the amino acid sequence of SEQ ID NO: 434; or
b. a Heavy Chain Variable Region (VH) comprising the amino acid sequence of SEQ ID NO: 460, or having at least 93%, 94%, 95%, 96%, 97%, 98% or 99% sequence identity to the amino acid sequence of SEQ ID NO: 460 and a Light Chain Variable Region (VL) comprising the amino acid sequence of SEQ ID NO: 434; or
c. a Heavy Chain Variable Region (VH) comprising the amino acid sequence of SEQ ID NO: 520, or having at least 93%, 94%, 95%, 96%, 97%, 98% or 99% sequence identity to the amino acid sequence of SEQ ID NO: 520 and a Light Chain Variable Region (VL) comprising the amino acid sequence of SEQ ID NO: 434; or
d. a Heavy Chain Variable Region (VH) comprising the amino acid sequence of SEQ ID NO: 480, or having at least 93%, 94%, 95%, 96%, 97%, 98% or 99% sequence identity to the amino acid sequence of SEQ ID NO: 480 and a Light Chain Variable Region (VL) comprising the amino acid sequence of SEQ ID NO: 424;
or comprising:
a. a Heavy Chain Variable Region (VH) comprising the amino acid sequence of SEQ ID NO: 440 and a Light Chain Variable Region (VL) comprising the amino acid sequence of SEQ ID NO: 434; or
b. a Heavy Chain Variable Region (VH) comprising the amino acid sequence of SEQ ID NO: 460 and a Light Chain Variable Region (VL) comprising the amino acid sequence of SEQ ID NO: 434; or
c. a Heavy Chain Variable Region (VH) comprising the amino acid sequence of SEQ ID NO: 520 and a Light Chain Variable Region (VL) comprising the amino acid sequence of SEQ ID NO: 434; or
d. a Heavy Chain Variable Region (VH) comprising the amino acid sequence of SEQ ID NO: 480 and a Light Chain Variable Region (VL) comprising the amino acid sequence of SEQ ID NO: 424.

98. The ASC binding molecule of claim 83, comprising:
a. a VH-CDR1 comprising the amino acid sequence of SEQ ID NO: 11, a VH-CDR2 comprising the amino acid sequence of SEQ ID NO: 12, a VH-CDR3 comprising the amino acid sequence NEV (Asn-Glu-Val), a VL-CDR1 comprising the amino sequence SEQ ID NO: 15, a VL-CDR2 comprising the amino acid sequence of SEQ ID NO: 16, and a VL-CDR3 comprising the amino acid sequence of SEQ ID NO: 17; or
b. a VH-CDR1 comprising the amino acid sequence of SEQ ID NO: 21, a VH-CDR2 comprising the amino acid sequence of SEQ ID NO: 22, a VH-CDR3 comprising the amino acid sequence SEQ ID NO: 23, a VL-CDR1 comprising the amino sequence SEQ ID NO: 25, a VL-CDR2 comprising the amino acid sequence of SEQ ID NO: 26, and a VL-CDR3 comprising the amino acid sequence of SEQ ID NO: 27; or
c. a VH-CDR1 comprising the amino acid sequence of SEQ ID NO: 31, a VH-CDR2 comprising the amino acid sequence of SEQ ID NO: 32, a VH-CDR3 comprising the amino acid sequence of SEQ ID NO: 33, a VL-CDR1 comprising the amino sequence SEQ ID NO: 35, a VL-CDR2 comprising the amino acid sequence of SEQ ID NO: 36, and a VL-CDR3 comprising the amino acid sequence of SEQ ID NO: 37; or
d. a VH-CDR1 comprising the amino acid sequence of SEQ ID NO: 41, a VH-CDR2 comprising the amino acid sequence of SEQ ID NO: 42, a VH-CDR3 comprising the amino acid sequence SEQ ID NO: 43, a VL-CDR1 comprising the amino sequence SEQ ID NO: 45, a VL-CDR2 comprising the amino acid sequence of SEQ ID NO: 46, and a VL-CDR3 comprising the amino acid sequence of SEQ ID NO: 47; or
e. a VH-CDR1 comprising the amino acid sequence of SEQ ID NO: 51, a VH-CDR2 comprising the amino acid sequence of SEQ ID NO: 52, a VH-CDR3 comprising the amino acid sequence SEQ ID NO: 53, a VL-CDR1 comprising the amino sequence SEQ ID NO: 55, a VL-CDR2 comprising the amino acid sequence of SEQ ID NO: 56, and a VL-CDR3 comprising the amino acid sequence of SEQ ID NO: 57; or
f. a VH-CDR1 comprising the amino acid sequence of SEQ ID NO: 61, a VH-CDR2 comprising the amino acid sequence of SEQ ID NO: 62, a VH-CDR3 comprising the amino acid sequence SEQ ID NO: 63, a VL-CDR1 comprising the amino sequence SEQ ID NO: 65, a VL-CDR2 comprising the amino acid sequence of SEQ ID NO: 66, and a VL-CDR3 comprising the amino acid sequence of SEQ ID NO: 67; or
g. a VH-CDR1 comprising the amino acid sequence of SEQ ID NO: 71, a VH-CDR2 comprising the amino acid sequence of SEQ ID NO: 72, a VH-CDR3 comprising the amino acid sequence SEQ ID NO: 73, a VL-CDR1 comprising the amino sequence SEQ ID NO: 75, a VL-CDR2 comprising the amino acid sequence of SEQ ID NO: 76, and a VL-CDR3 comprising the amino acid sequence of SEQ ID NO: 77; or
h. a VH-CDR1 comprising the amino acid sequence of SEQ ID NO: 81, a VH-CDR2 comprising the amino acid sequence of SEQ ID NO: 82, a VH-CDR3 comprising the amino acid sequence SEQ ID NO: 83, a VL-CDR1 comprising the amino sequence SEQ ID NO: 85, a VL-CDR2 comprising the amino acid sequence of SEQ ID NO: 86, and a VL-CDR3 comprising the amino acid sequence of SEQ ID NO: 87; or
i. a VH-CDR1 comprising the amino acid sequence of SEQ ID NO: 91, a VH-CDR2 comprising the amino acid sequence of SEQ ID NO: 92, a VH-CDR3 comprising the amino acid sequence SEQ ID NO: 93, a VL-CDR1 comprising the amino sequence SEQ ID NO: 95, a VL-CDR2 comprising the amino acid sequence of SEQ ID NO: 96, and a VL-CDR3 comprising the amino acid sequence of SEQ ID NO: 97; or
j. a VH-CDR1 comprising the amino acid sequence of SEQ ID NO: 111, a VH-CDR2 comprising the amino acid sequence of SEQ ID NO: 112, a VH-CDR3 comprising the amino acid sequence SEQ ID NO: 113, a VL-CDR1 comprising the amino sequence SEQ ID NO: 115, a VL-CDR2 comprising the amino acid sequence of SEQ ID NO: 116, and a VL-CDR3 comprising the amino acid sequence of SEQ ID NO: 117; or
k. a VH-CDR1 comprising the amino acid sequence of SEQ ID NO: 121, a VH-CDR2 comprising the amino acid sequence of SEQ ID NO: 122, a VH-CDR3 comprising the amino acid sequence SEQ ID NO: 123, a VL-CDR1 comprising the amino sequence SEQ ID NO: 125, a VL-CDR2 comprising the amino acid sequence of SEQ ID NO: 126, and a VL-CDR3 comprising the amino acid sequence of SEQ ID NO: 127; or
l. a VH-CDR1 comprising the amino acid sequence of SEQ ID NO: 131, a VH-CDR2 comprising the amino acid sequence of SEQ ID NO: 132, a VH-CDR3 comprising the amino acid sequence SEQ ID NO: 133, a VL-CDR1 comprising the amino sequence SEQ ID NO: 135, a VL-CDR2 comprising the amino acid sequence of SEQ ID NO: 66, and a VL-CDR3 comprising the amino acid sequence of SEQ ID NO: 137; or
m. a VH-CDR1 comprising the amino acid sequence of SEQ ID NO: 111, a VH-CDR2 comprising the amino acid sequence of SEQ ID NO: 142, a VH-CDR3 comprising the amino acid sequence SEQ ID NO: 143, a VL-CDR1 comprising the amino sequence SEQ ID NO: 145, a VL-CDR2 comprising the amino acid sequence of SEQ ID NO: 66, and a VL-CDR3 comprising the amino acid sequence of SEQ ID NO: 147; or
n. a VH-CDR1 comprising the amino acid sequence of SEQ ID NO: 151, a VH-CDR2 comprising the amino acid sequence of SEQ ID NO: 152, a VH-CDR3 comprising the amino acid sequence SEQ ID NO: 153, a VL-CDR1 comprising the amino sequence SEQ ID NO: 155, a VL-CDR2 comprising the amino acid sequence of SEQ ID NO: 156, and a VL-CDR3 comprising the amino acid sequence of SEQ ID NO: 157; or
o. a VH-CDR1 comprising the amino acid sequence of SEQ ID NO: 161, a VH-CDR2 comprising the amino acid sequence of SEQ ID NO: 162, a VH-CDR3 comprising the amino acid sequence SEQ ID NO: 163, a VL-CDR1 comprising the amino sequence SEQ ID NO: 165, a VL-CDR2 comprising the amino acid sequence of SEQ ID NO: 166, and a VL-CDR3 comprising the amino acid sequence of SEQ ID NO: 167; or
p. a VH-CDR1 comprising the amino acid sequence of SEQ ID NO: 171, a VH-CDR2 comprising the amino acid sequence of SEQ ID NO: 172, a VH-CDR3 comprising the amino acid sequence SEQ ID NO: 173, a VL-CDR1 comprising the amino sequence SEQ ID NO: 175, a VL-CDR2 comprising the amino acid sequence of SEQ ID NO: 176, and a VL-CDR3 comprising the amino acid sequence of SEQ ID NO: 177; or
q. a VH-CDR1 comprising the amino acid sequence of SEQ ID NO: 181, a VH-CDR2 comprising the amino acid sequence of SEQ ID NO: 182, a VH-CDR3 comprising the amino acid sequence SEQ ID NO: 183, a VL-CDR1 comprising the amino sequence SEQ ID NO: 185, a VL-CDR2 comprising the amino acid sequence of SEQ ID NO: 186, and a VL-CDR3 comprising the amino acid sequence of SEQ ID NO: 187; or
r. a VH-CDR1 comprising the amino acid sequence of SEQ ID NO: 191, a VH-CDR2 comprising the amino acid sequence of SEQ ID NO: 192, a VH-CDR3 comprising the amino acid sequence SEQ ID NO: 193, a VL-CDR1 comprising the amino sequence SEQ ID NO: 195, a VL-CDR2 comprising the amino acid sequence of SEQ ID NO: 196, and a VL-CDR3 comprising the amino acid sequence of SEQ ID NO: 197; or
s. a VH-CDR1 comprising the amino acid sequence of SEQ ID NO: 201, a VH-CDR2 comprising the amino acid sequence of SEQ ID NO: 202, a VH-CDR3 comprising the amino acid sequence SEQ ID NO: 203, a VL-CDR1 comprising the amino sequence SEQ ID NO: 205, a VL-CDR2 comprising the amino acid sequence of SEQ ID NO: 206, and a VL-CDR3 comprising the amino acid sequence of SEQ ID NO: 207; or
t. a VH-CDR1 comprising the amino acid sequence of SEQ ID NO: 211, a VH-CDR2 comprising the amino acid sequence of SEQ ID NO: 212, a VH-CDR3 comprising the amino acid sequence SEQ ID NO: 213, a VL-CDR1 comprising the amino sequence SEQ ID NO: 215, a VL-CDR2 comprising the amino acid sequence of SEQ ID NO: 186, and a VL-CDR3 comprising the amino acid sequence of SEQ ID NO: 217; or
u. a VH-CDR1 comprising the amino acid sequence of SEQ ID NO: 221, a VH-CDR2 comprising the amino acid sequence of SEQ ID NO: 222, a VH-CDR3 comprising the amino acid sequence SEQ ID NO: 223, a VL-CDR1 comprising the amino sequence SEQ ID NO: 225, a VL-CDR2 comprising the amino acid sequence of SEQ ID NO: 226, and a VL-CDR3 comprising the amino acid sequence of SEQ ID NO: 227; or
v. a VH-CDR1 comprising the amino acid sequence of SEQ ID NO: 231, a VH-CDR2 comprising the amino acid sequence of SEQ ID NO: 232, a VH-CDR3 comprising the amino acid sequence SEQ ID NO: 233, a VL-CDR1 comprising the amino sequence SEQ ID NO: 235, a VL-CDR2 comprising the amino acid sequence of SEQ ID NO: 236, and a VL-CDR3 comprising the amino acid sequence of SEQ ID NO: 237; or
w. a VH-CDR1 comprising the amino acid sequence of SEQ ID NO: 241, a VH-CDR2 comprising the amino acid sequence of SEQ ID NO: 152, a VH-CDR3 comprising the amino acid sequence SEQ ID NO: 243, a VL-CDR1 comprising the amino sequence SEQ ID NO: 245, a VL-CDR2 comprising the amino acid sequence of SEQ ID NO: 246, and a VL-CDR3 comprising the amino acid sequence of SEQ ID NO: 247; or
x. a VH-CDR1 comprising the amino acid sequence of SEQ ID NO: 251, a VH-CDR2 comprising the amino acid sequence of SEQ ID NO: 252, a VH-CDR3 comprising the amino acid sequence SEQ ID NO: 253, a VL-CDR1 comprising the amino sequence SEQ ID NO: 255, a VL-CDR2 comprising the amino acid sequence of SEQ ID NO: 256, and a VL-CDR3 comprising the amino acid sequence of SEQ ID NO: 257; or
y. a VH-CDR1 comprising the amino acid sequence of SEQ ID NO: 261, a VH-CDR2 comprising the amino acid sequence of SEQ ID NO: 262, a VH-CDR3 comprising the amino acid sequence SEQ ID NO: 263, a VL-CDR1 comprising the amino sequence SEQ ID NO: 265, a VL-CDR2 comprising the amino acid sequence of SEQ ID NO: 266, and a VL-CDR3 comprising the amino acid sequence of SEQ ID NO: 267; or
z. a VH-CDR1 comprising the amino acid sequence of SEQ ID NO: 271, a VH-CDR2 comprising the amino acid sequence of SEQ ID NO: 272, a VH-CDR3 comprising the amino acid sequence SEQ ID NO: 203, a VL-CDR1 comprising the amino sequence SEQ ID NO: 275, a VL-CDR2 comprising the amino acid sequence of SEQ ID NO: 276, and a VL-CDR3 comprising the amino acid sequence of SEQ ID NO: 207; or
aa. a VH-CDR1 comprising the amino acid sequence of SEQ ID NO: 281, a VH-CDR2 comprising the amino acid sequence of SEQ ID NO: 152, a VH-CDR3 comprising the amino acid sequence SEQ ID NO: 283, a VL-CDR1 comprising the amino sequence SEQ ID NO: 285, a VL-CDR2 comprising the amino acid sequence of SEQ ID NO: 286, and a VL-CDR3 comprising the amino acid sequence of SEQ ID NO: 287; or
bb. a VH-CDR1 comprising the amino acid sequence of SEQ ID NO: 291, a VH-CDR2 comprising the amino acid sequence of SEQ ID NO: 292, a VH-CDR3 comprising the amino acid sequence SEQ ID NO: 293, a VL-CDR1 comprising the amino sequence SEQ ID NO: 295, a VL-CDR2 comprising the amino acid sequence of SEQ ID NO: 26, and a VL-CDR3 comprising the amino acid sequence of SEQ ID NO: 297; or
cc. a VH-CDR1 comprising the amino acid sequence of SEQ ID NO: 71, a VH-CDR2 comprising the amino acid sequence of SEQ ID NO: 302, a VH-CDR3 comprising the amino acid sequence SEQ ID NO: 303, a VL-CDR1 comprising the amino sequence SEQ ID NO: 305, a VL-CDR2 comprising the amino acid sequence of SEQ ID NO: 126, and a VL-CDR3 comprising the amino acid sequence of SEQ ID NO: 307; or
dd. a VH-CDR1 comprising the amino acid sequence of SEQ ID NO: 161, a VH-CDR2 comprising the amino acid sequence of SEQ ID NO: 312, a VH-CDR3 comprising the amino acid sequence RDY (Arg-Asp-Tyr), a VL-CDR1 comprising the amino sequence SEQ ID NO: 315, a VL-CDR2 comprising the amino acid sequence of SEQ ID NO: 186, and a VL-CDR3 comprising the amino acid sequence of SEQ ID NO: 317; or
ee. a VH-CDR1 comprising the amino acid sequence of SEQ ID NO: 161, a VH-CDR2 comprising the amino acid sequence of SEQ ID NO: 322, a VH-CDR3 comprising the amino acid sequence SEQ ID NO: 323, a VL-CDR1 comprising the amino sequence SEQ ID NO: 205, a VL-CDR2 comprising the amino acid sequence of SEQ ID NO: 326, and a VL-CDR3 comprising the amino acid sequence of SEQ ID NO: 327; or
ff. a VH-CDR1 comprising the amino acid sequence of SEQ ID NO: 331, a VH-CDR2 comprising the amino acid sequence of SEQ ID NO: 332, a VH-CDR3 comprising the amino acid sequence SEQ ID NO: 333, a VL-CDR1 comprising the amino sequence SEQ ID NO: 335, a VL-CDR2 comprising the amino acid sequence of SEQ ID NO: 336, and a VL-CDR3 comprising the amino acid sequence of SEQ ID NO: 337; or
gg. a VH-CDR1 comprising the amino acid sequence of SEQ ID NO: 161, a VH-CDR2 comprising the amino acid sequence of SEQ ID NO: 342, a VH-CDR3 comprising the amino acid sequence SEQ ID NO: 323, a VL-CDR1 comprising the amino sequence SEQ ID NO: 205, a VL-CDR2 comprising the amino acid sequence of SEQ ID NO: 326, and a VL-CDR3 comprising the amino acid sequence of SEQ ID NO: 347; or
hh. a VH-CDR1 comprising the amino acid sequence of SEQ ID NO: 331, a VH-CDR2 comprising the amino acid sequence of SEQ ID NO: 352, a VH-CDR3 comprising the amino acid sequence SEQ ID NO: 353, a VL-CDR1 comprising the amino sequence SEQ ID NO: 205, a VL-CDR2 comprising the amino acid sequence of SEQ ID NO: 336, and a VL-CDR3 comprising the amino acid sequence of SEQ ID NO: 337; or
ii. a VH-CDR1 comprising the amino acid sequence of SEQ ID NO: 361, a VH-CDR2 comprising the amino acid sequence of SEQ ID NO: 362, a VH-CDR3 comprising the amino acid sequence RDY (Arg-Asp-Tyr), a VL-CDR1 comprising the amino sequence SEQ ID NO: 315, a VL-CDR2 comprising the amino acid sequence of SEQ ID NO: 186, and a VL-CDR3 comprising the amino acid sequence of SEQ ID NO: 367; or
jj. a VH-CDR1 comprising the amino acid sequence of SEQ ID NO: 371, a VH-CDR2 comprising the amino acid sequence of SEQ ID NO: 372, a VH-CDR3 comprising the amino acid sequence SEQ ID NO: 373, a VL-CDR1 comprising the amino sequence SEQ ID NO: 375, a VL-CDR2 comprising the amino acid sequence of SEQ ID NO: 376, and a VL-CDR3 comprising the amino acid sequence of SEQ ID NO: 377; or
kk. a VH-CDR1 comprising the amino acid sequence of SEQ ID NO: 381, a VH-CDR2 comprising the amino acid sequence of SEQ ID NO: 382, a VH-CDR3 comprising the amino acid sequence SEQ ID NO: 383, a VL-CDR1 comprising the amino sequence SEQ ID NO: 385, a VL-CDR2 comprising the amino acid sequence of SEQ ID NO: 386, and a VL-CDR3 comprising the amino acid sequence of SEQ ID NO: 387; or
ll. a VH-CDR1 comprising the amino acid sequence of SEQ ID NO: 271, a VH-CDR2 comprising the amino acid sequence of SEQ ID NO: 392, a VH-CDR3 comprising the amino acid sequence SEQ ID NO: 393, a VL-CDR1 comprising the amino sequence SEQ ID NO: 335, a VL-CDR2 comprising the amino acid sequence of SEQ ID NO: 276, and a VL-CDR3 comprising the amino acid sequence of SEQ ID NO: 207.

99. The ASC binding molecule of claim 83, comprising:
a. a Heavy Chain Variable Region (VH) comprising the amino acid sequence of SEQ ID NO: 10 or a Heavy Chain Variable Region (VH) having at least 92%, 93%, 94%, 95%, 96%, 97%, 98% or 99% sequence identity to the amino acid sequence of SEQ ID NO: 10; and a Light Chain Variable Region (VL) comprising the amino acid sequence of SEQ ID NO: 14 or a Light Chain Variable Region (VL) having at least 98% or 99% sequence identity to the amino acid sequence of SEQ ID NO: 14; or
b. a Heavy Chain Variable Region (VH) comprising the amino acid sequence of SEQ ID NO: 20 or a Heavy Chain Variable Region (VH) having at least 90%, 91%, 92%, 93%, 94%, 95%, 96%, 97%, 98% or 99% sequence identity to the amino acid sequence of SEQ ID NO: 20; and a Light Chain Variable Region (VL) comprising the amino acid sequence of SEQ ID NO: 24 or a Light Chain Variable Region (VL) having at least 95%, 96%, 97%, 98% or 99% sequence identity to the amino acid sequence of SEQ ID NO: 24; or
c. a Heavy Chain Variable Region (VH) comprising the amino acid sequence of SEQ ID NO: 30 or a Heavy Chain Variable Region (VH) having at least 85%, 86%, 87%, 88%, 89%, 90%, 91%, 92%, 93%, 94%, 95%, 96%, 97%, 98% or 99% sequence identity to the amino acid sequence of SEQ ID NO: 30; and a Light Chain Variable Region (VL) comprising the amino acid sequence of SEQ ID NO: 34 or a Light Chain Variable Region (VL) having at least 93%, 94%, 95%, 96%, 97%, 98% or 99% sequence identity to the amino acid sequence of SEQ ID NO: 34; or
d. a Heavy Chain Variable Region (VH) comprising the amino acid sequence of SEQ ID NO: 40 or a Heavy Chain Variable Region (VH) having at least 86%, 87%, 88%, 89%, 90%, 91%, 92%, 93%, 94%, 95%, 96%, 97%, 98% or 99% sequence identity to the amino acid sequence of SEQ ID NO: 40; and a Light Chain Variable Region (VL) comprising the amino acid sequence of SEQ ID NO: 44 or a Light Chain Variable Region (VL) having at least 96%, 97%, 98% or 99% sequence identity to the amino acid sequence of SEQ ID NO: 44; or
e. a Heavy Chain Variable Region (VH) comprising the amino acid sequence of SEQ ID NO: 50 or a Heavy Chain Variable Region (VH) having at least 94%, 95%, 96%, 97%, 98% or 99% sequence identity to the amino acid sequence of SEQ ID NO: 50; and a Light Chain Variable Region (VL) comprising the amino acid sequence of SEQ ID NO: 54 or a Light Chain Variable Region (VL) having at least 96%, 97%, 98% or 99% sequence identity to the amino acid sequence of SEQ ID NO: 54; or
f. a Heavy Chain Variable Region (VH) comprising the amino acid sequence of SEQ ID NO: 60 or a Heavy Chain Variable Region (VH) having at least 85%, 86%, 87%, 88%, 95%, 96%, 97%, 98% or 99% sequence identity to the amino acid sequence of SEQ ID NO: 60; and a Light Chain Variable Region (VL) comprising the amino acid sequence of SEQ ID NO: 64; or
g. a Heavy Chain Variable Region (VH) comprising the amino acid sequence of SEQ ID NO: 70 or a Heavy Chain Variable Region (VH) having at least 90%, 91%, 92%, 93%, 94%, 95%, 96%, 97%, 98% or 99% sequence identity to the amino acid sequence of SEQ ID NO: 70; and a Light Chain Variable Region (VL) comprising the amino acid sequence of SEQ ID NO: 74 or a Light Chain Variable Region (VL) having at least 97%, 98% or 99% sequence identity to the amino acid sequence of SEQ ID NO: 74; or
h. a Heavy Chain Variable Region (VH) comprising the amino acid sequence of SEQ ID NO: 80 or a Heavy Chain Variable Region (VH) having at least 93%, 94%, 95%, 96%, 97%, 98% or 99% sequence identity to the amino acid sequence of SEQ ID NO: 80; and a Light Chain Variable Region (VL) comprising the amino acid sequence of SEQ ID NO: 84 or a Light Chain Variable Region (VL) having at least 95%, 96%, 97%, 98% or 99% sequence identity to the amino acid sequence of SEQ ID NO: 84; or
i. a Heavy Chain Variable Region (VH) comprising the amino acid sequence of SEQ ID NO: 90 or a Heavy Chain Variable Region (VH) having at least 93%, 94%, 95%, 96%, 97%, 98% or 99% sequence identity to the amino acid sequence of SEQ ID NO: 90; and a Light Chain Variable Region (VL) comprising the amino acid sequence of SEQ ID NO: 94 or a Light Chain Variable Region (VL) having at least 93%, 94%, 95%, 96%, 97%, 98% or 99% sequence identity to the amino acid sequence of SEQ ID NO: 94; or
j. a Heavy Chain Variable Region (VH) comprising the amino acid sequence of SEQ ID NO: 110 or a Heavy Chain Variable Region (VH) having at least 88%, 89%, 90%, 91%, 92%, 93%, 94%, 95%, 96%, 97%, 98% or 99% sequence identity to the amino acid sequence of SEQ ID NO: 110; and a Light Chain Variable Region (VL) comprising the amino acid sequence of SEQ ID NO: 114 or a Light Chain Variable Region (VL) having at least 98% or 99% sequence identity to the amino acid sequence of SEQ ID NO: 114; or
k. a Heavy Chain Variable Region (VH) comprising the amino acid sequence of SEQ ID NO: 120 or a Heavy Chain Variable Region (VH) having at least 92%, 93%, 94%, 95%, 96%, 97%, 98% or 99% sequence identity to the amino acid sequence of SEQ ID NO: 120; and a Light Chain Variable Region (VL) comprising the amino acid sequence of SEQ ID NO: 124 or a Light Chain Variable Region (VL) having at least 98% or 99% sequence identity to the amino acid sequence of SEQ ID NO: 124; or
l. a Heavy Chain Variable Region (VH) comprising the amino acid sequence of SEQ ID NO: 130 or a Heavy Chain Variable Region (VH) having at least 87%, 88%, 89%, 90%, 91%, 92%, 93%, 94%, 95%, 96%, 97%, 98% or 99% sequence identity to the amino acid sequence of SEQ ID NO: 130; and a Light Chain Variable Region (VL) comprising the amino acid sequence of SEQ ID NO: 134 or a Light Chain Variable Region (VL) having at least 98% or 99% sequence identity to the amino acid sequence of SEQ ID NO: 134; or
m. a Heavy Chain Variable Region (VH) comprising the amino acid sequence of SEQ ID NO: 140 or a Heavy Chain Variable Region (VH) having at least 94%, 95%, 96%, 97%, 98% or 99% sequence identity to the amino acid sequence of SEQ ID NO: 140; and a Light Chain Variable Region (VL) comprising the amino acid sequence of SEQ ID NO: 144 or a Light Chain Variable Region (VL) having at least 98% or 99% sequence identity to the amino acid sequence of SEQ ID NO: 144; or
n. a Heavy Chain Variable Region (VH) comprising the amino acid sequence of SEQ ID NO: 150 or a Heavy Chain Variable Region (VH) having at least 90%, 91%, 92%, 93%, 94%, 95%, 96%, 97%, 98% or 99% sequence identity to the amino acid sequence of SEQ ID NO: 150; and a Light Chain Variable Region (VL) comprising the amino acid sequence of SEQ ID NO: 154 or a Light Chain Variable Region (VL) having at least 93%, 94%, 95%, 96%, 97%, 98% or 99% sequence identity to the amino acid sequence of SEQ ID NO: 154; or
o. a Heavy Chain Variable Region (VH) comprising the amino acid sequence of SEQ ID NO: 160 or a Heavy Chain Variable Region (VH) having at least 88%, 89%, 90%, 91%, 92%, 93%, 94%, 95%, 96%, 97%, 98% or 99% sequence identity to the amino acid sequence of SEQ ID NO: 160; and a Light Chain Variable Region (VL) comprising the amino acid sequence of SEQ ID NO: 164 or a Light Chain Variable Region (VL) having at least 96%, 97%, 98% or 99% sequence identity to the amino acid sequence of SEQ ID NO: 164; or
p. a Heavy Chain Variable Region (VH) comprising the amino acid sequence of SEQ ID NO: 170 or a Heavy Chain Variable Region (VH) having at least 85%, 86%, 87%, 88%, 89%, 90%, 91%, 92%, 93%, 94%, 95%, 96%, 97%, 98% or 99% sequence identity to the amino acid sequence of SEQ ID NO: 170; and a Light Chain Variable Region (VL) comprising the amino acid sequence of SEQ ID NO: 174 or a Light Chain Variable Region (VL) having at least 93%, 94%, 95%, 96%, 97%, 98% or 99% sequence identity to the amino acid sequence of SEQ ID NO: 174; or
q. a Heavy Chain Variable Region (VH) comprising the amino acid sequence of SEQ ID NO: 180 or a Heavy Chain Variable Region (VH) having at least 94%, 95%, 96%, 97%, 98% or 99% sequence identity to the amino acid sequence of SEQ ID NO: 180; and a Light Chain Variable Region (VL) comprising the amino acid sequence of SEQ ID NO: 184 or a Light Chain Variable Region (VL) having at least 97%, 98% or 99% sequence identity to the amino acid sequence of SEQ ID NO: 184; or
r. a Heavy Chain Variable Region (VH) comprising the amino acid sequence of SEQ ID NO: 190 or a Heavy Chain Variable Region (VH) having at least 87%, 88%, 89%, 90%, 91%, 92%, 93%, 94%, 95%, 96%, 97%, 98% or 99% sequence identity to the amino acid sequence of SEQ ID NO: 190; and a Light Chain Variable Region (VL) comprising the amino acid sequence of SEQ ID NO: 194; or
s. a Heavy Chain Variable Region (VH) comprising the amino acid sequence of SEQ ID NO: 200 or a Heavy Chain Variable Region (VH) having at least 93%, 94%, 95%, 96%, 97%, 98% or 99% sequence identity to the amino acid sequence of SEQ ID NO: 200; and a Light Chain Variable Region (VL) comprising the amino acid sequence of SEQ ID NO: 204 or a Light Chain Variable Region (VL) having at least 99% sequence identity to the amino acid sequence of SEQ ID NO: 204; or
t. a Heavy Chain Variable Region (VH) comprising the amino acid sequence of SEQ ID NO: 210 or a Heavy Chain Variable Region (VH) having at least 92%, 93%, 94%, 95%, 96%, 97%, 98% or 99% sequence identity to the amino acid sequence of SEQ ID NO: 210; and a Light Chain Variable Region (VL) comprising the amino acid sequence of SEQ ID NO: 214 or a Light Chain Variable Region (VL) having at least 96%, 97%, 98% or 99% sequence identity to the amino acid sequence of SEQ ID NO: 214; or
u. a Heavy Chain Variable Region (VH) comprising the amino acid sequence of SEQ ID NO: 220 or a Heavy Chain Variable Region (VH) having at least 84%, 85%, 86%, 87%, 88%, 89%, 90%, 91%, 92%, 93%, 94%, 95%, 96%, 97%, 98% or 99% sequence identity to the amino acid sequence of SEQ ID NO: 220; and a Light Chain Variable Region (VL) comprising the amino acid sequence of SEQ ID NO: 224 or a Light Chain Variable Region (VL) having at least 93%, 94%, 95%, 96%, 97%, 98% or 99% sequence identity to the amino acid sequence of SEQ ID NO: 224; or
v. a Heavy Chain Variable Region (VH) comprising the amino acid sequence of SEQ ID NO: 230 or a Heavy Chain Variable Region (VH) having at least 84%, 85%, 86%, 87%, 88%, 89%, 90%, 91%, 92%, 93%, 94%, 95%, 96%, 97%, 98% or 99% sequence identity to the amino acid sequence of SEQ ID NO: 230; and a Light Chain Variable Region (VL) comprising the amino acid sequence of SEQ ID NO: 234 or a Light Chain Variable Region (VL) having at least 92%, 93%, 94%, 95%, 96%, 97%, 98% or 99% sequence identity to the amino acid sequence of SEQ ID NO: 234; or
w. a Heavy Chain Variable Region (VH) comprising the amino acid sequence of SEQ ID NO: 240 or a Heavy Chain Variable Region (VH) having at least 88%, 89%, 90%, 91%, 92%, 93%, 94%, 95%, 96%, 97%, 98% or 99% sequence identity to the amino acid sequence of SEQ ID NO: 240; and a Light Chain Variable Region (VL) comprising the amino acid sequence of SEQ ID NO: 244 or a Light Chain Variable Region (VL) having at least 97%, 98% or 99% sequence identity to the amino acid sequence of SEQ ID NO: 244; or
x. a Heavy Chain Variable Region (VH) comprising the amino acid sequence of SEQ ID NO: 250 or a Heavy Chain Variable Region (VH) having at least 89%, 90%, 91%, 92%, 93%, 94%, 95%, 96%, 97%, 98% or 99% sequence identity to the amino acid sequence of SEQ ID NO: 250; and a Light Chain Variable Region (VL) comprising the amino acid sequence of SEQ ID NO: 254 or a Light Chain Variable Region (VL) having at least 97%, 98% or 99% sequence identity to the amino acid sequence of SEQ ID NO: 254; or
y. a Heavy Chain Variable Region (VH) comprising the amino acid sequence of SEQ ID NO: 260 or a Heavy Chain Variable Region (VH) having at least 90%, 91%, 92%, 93%, 94%, 95%, 96%, 97%, 98% or 99% sequence identity to the amino acid sequence of SEQ ID NO: 260; and a Light Chain Variable Region (VL) comprising the amino acid sequence of SEQ ID NO: 264 or a Light Chain Variable Region (VL) having at least 97%, 98% or 99% sequence identity to the amino acid sequence of SEQ ID NO: 264; or
z. a Heavy Chain Variable Region (VH) comprising the amino acid sequence of SEQ ID NO: 270 or a Heavy Chain Variable Region (VH) having at least 91%, 92%, 93%, 94%, 95%, 96%, 97%, 98% or 99% sequence identity to the amino acid sequence of SEQ ID NO: 270; and a Light Chain Variable Region (VL) comprising the amino acid sequence of SEQ ID NO: 274 or a Light Chain Variable Region (VL) having at least or 99% sequence identity to the amino acid sequence of SEQ ID NO: 274; or
aa. a Heavy Chain Variable Region (VH) comprising the amino acid sequence of SEQ ID NO: 280 or a Heavy Chain Variable Region (VH) having at least 87%, 88%, 89%, 90%, 91%, 92%, 93%, 94%, 95%, 96%, 97%, 98% or 99% sequence identity to the amino acid sequence of SEQ ID NO: 280; and a Light Chain Variable Region (VL) comprising the amino acid sequence of SEQ ID NO: 284 or a Light Chain Variable Region (VL) having at least 98% or 99% sequence identity to the amino acid sequence of SEQ ID NO: 284; or
bb. a Heavy Chain Variable Region (VH) comprising the amino acid sequence of SEQ ID NO: 290 or a Heavy Chain Variable Region (VH) having at least 92%, 93%, 94%, 95%, 96%, 97%, 98% or 99% sequence identity to the amino acid sequence of SEQ ID NO: 290; and a Light Chain Variable Region (VL) comprising the amino acid sequence of SEQ ID NO: 294 or a Light Chain Variable Region (VL) having at least 99% sequence identity to the amino acid sequence of SEQ ID NO: 294; or
cc. a Heavy Chain Variable Region (VH) comprising the amino acid sequence of SEQ ID NO: 300 or a Heavy Chain Variable Region (VH) having at least 95%, 96%, 97%, 98% or 99% sequence identity to the amino acid sequence of SEQ ID NO: 300; and a Light Chain Variable Region (VL) comprising the amino acid sequence of SEQ ID NO: 304 or a Light Chain Variable Region (VL) having at least 98% or 99% sequence identity to the amino acid sequence of SEQ ID NO: 304; or
dd. a Heavy Chain Variable Region (VH) comprising the amino acid sequence of SEQ ID NO: 310 or a Heavy Chain Variable Region (VH) having at least 95%, 96%, 97%, 98% or 99% sequence identity to the amino acid sequence of SEQ ID NO: 310; and a Light Chain Variable Region (VL) comprising the amino acid sequence of SEQ ID NO: 314 or a Light Chain Variable Region (VL) having at least 97%, 98% or 99% sequence identity to the amino acid sequence of SEQ ID NO: 314; or
ee. a Heavy Chain Variable Region (VH) comprising the amino acid sequence of SEQ ID NO: 320 or a Heavy Chain Variable Region (VH) having at least 93%, 94%, 95%, 96%, 97%, 98% or 99% sequence identity to the amino acid sequence of SEQ ID NO: 320; and a Light Chain Variable Region (VL) comprising the amino acid sequence of SEQ ID NO: 324 or a Light Chain Variable Region (VL) having at least 94%, 95%, 96%, 97%, 98% or 99% sequence identity to the amino acid sequence of SEQ ID NO: 324; or
ff. a Heavy Chain Variable Region (VH) comprising the amino acid sequence of SEQ ID NO: 330 or a Heavy Chain Variable Region (VH) having at least 87%, 88%, 89%, 90%, 91%, 92%, 93%, 94%, 95%, 96%, 97%, 98% or 99% sequence identity to the amino acid sequence of SEQ ID NO: 330; and a Light Chain Variable Region (VL) comprising the amino acid sequence of SEQ ID NO: 334 or a Light Chain Variable Region (VL) having at least 96%, 97%, 98% or 99% sequence identity to the amino acid sequence of SEQ ID NO: 334; or
gg. a Heavy Chain Variable Region (VH) comprising the amino acid sequence of SEQ ID NO: 340 or a Heavy Chain Variable Region (VH) having at least 89%, 90%, 91%, 92%, 93%, 94%, 95%, 96%, 97%, 98% or 99% sequence identity to the amino acid sequence of SEQ ID NO: 340; and a Light Chain Variable Region (VL) comprising the amino acid sequence of SEQ ID NO: 344 or a Light Chain Variable Region (VL) having at least 89%, 90%, 91%, 92%, 93%, 94%, 95%, 96%, 97%, 98% or 99% sequence identity to the amino acid sequence of SEQ ID NO: 344; or
hh. a Heavy Chain Variable Region (VH) comprising the amino acid sequence of SEQ ID NO: 350 or a Heavy Chain Variable Region (VH) having at least 89%, 90%, 91%, 92%, 93%, 94%, 95%, 96%, 97%, 98% or 99% sequence identity to the amino acid sequence of SEQ ID NO: 350; and a Light Chain Variable Region (VL) comprising the amino acid sequence of SEQ ID NO: 354 or a Light Chain Variable Region (VL) having at least 96%, 97%, 98% or 99% sequence identity to the amino acid sequence of SEQ ID NO: 354; or
ii. a Heavy Chain Variable Region (VH) comprising the amino acid sequence of SEQ ID NO: 360 or a Heavy Chain Variable Region (VH) having at least 92%, 93%, 94%, 95%, 96%, 97%, 98% or 99% sequence identity to the amino acid sequence of SEQ ID NO: 360; and a Light Chain Variable Region (VL) comprising the amino acid sequence of SEQ ID NO: 364 or a Light Chain Variable Region (VL) having at least 98% or 99% sequence identity to the amino acid sequence of SEQ ID NO: 364; or
jj. a Heavy Chain Variable Region (VH) comprising the amino acid sequence of SEQ ID NO: 370 or a Heavy Chain Variable Region (VH) having at least 85%, 86%, 87%, 88%, 89%, 90%, 91%, 92%, 93%, 94%, 95%, 96%, 97%, 98% or 99% sequence identity to the amino acid sequence of SEQ ID NO: 370; and a Light Chain Variable Region (VL) comprising the amino acid sequence of SEQ ID NO: 374 or a Light Chain Variable Region (VL) having at least 94%, 95%, 96%, 97%, 98% or 99% sequence identity to the amino acid sequence of SEQ ID NO: 374; or
kk. a Heavy Chain Variable Region (VH) comprising the amino acid sequence of SEQ ID NO: 380 or a Heavy Chain Variable Region (VH) having at least 86%, 87%, 88%, 89%, 90%, 91%, 92%, 93%, 94%, 95%, 96%, 97%, 98% or 99% sequence identity to the amino acid sequence of SEQ ID NO: 380; and a Light Chain Variable Region (VL) comprising the amino acid sequence of SEQ ID NO: 384 or a Light Chain Variable Region (VL) having at least 93%, 94%, 95%, 96%, 97%, 98% or 99% sequence identity to the amino acid sequence of SEQ ID NO: 384; or
ll. a Heavy Chain Variable Region (VH) comprising the amino acid sequence of SEQ ID NO: 390 or a Heavy Chain Variable Region (VH) having at least 95%, 96%, 97%, 98% or 99% sequence identity to the amino acid sequence of SEQ ID NO: 390; and a Light Chain Variable Region (VL) comprising the amino acid sequence of SEQ ID NO: 394.

100. The ASC binding molecule of claim 83, comprising:
a. a Heavy Chain Variable Region (VH) comprising the amino acid sequence of SEQ ID NO: 10 and a Light Chain Variable Region (VL) comprising the amino acid sequence of SEQ ID NO: 14; or
b. a Heavy Chain Variable Region (VH) comprising the amino acid sequence of SEQ ID NO: 20; and a Light Chain Variable Region (VL) comprising the amino acid sequence of SEQ ID NO: 24; or
c. a Heavy Chain Variable Region (VH) comprising the amino acid sequence of SEQ ID NO: 30 and a Light Chain Variable Region (VL) comprising the amino acid sequence of SEQ ID NO: 14; or
d. a Heavy Chain Variable Region (VH) comprising the amino acid sequence of SEQ ID NO: 40 and a Light Chain Variable Region (VL) comprising the amino acid sequence of SEQ ID NO: 44; or
e. a Heavy Chain Variable Region (VH) comprising the amino acid sequence of SEQ ID NO: 50; and a Light Chain Variable Region (VL) comprising the amino acid sequence of SEQ ID NO: 54; or
f. a Heavy Chain Variable Region (VH) comprising the amino acid sequence of SEQ ID NO: 60 and a Light Chain Variable Region (VL) comprising the amino acid sequence of SEQ ID NO: 64; or
g. a Heavy Chain Variable Region (VH) comprising the amino acid sequence of SEQ ID NO: 70 and a Light Chain Variable Region (VL) comprising the amino acid sequence of SEQ ID NO: 74; or
h. a Heavy Chain Variable Region (VH) comprising the amino acid sequence of SEQ ID NO: 80 and a Light Chain Variable Region (VL) comprising the amino acid sequence of SEQ ID NO: 84; or
i. a Heavy Chain Variable Region (VH) comprising the amino acid sequence of SEQ ID NO: 90 and a Light Chain Variable Region (VL) comprising the amino acid sequence of SEQ ID NO: 94; or
j. a Heavy Chain Variable Region (VH) comprising the amino acid sequence of SEQ ID NO: 110 and a Light Chain Variable Region (VL) comprising the amino acid sequence of SEQ ID NO: 114; or
k. a Heavy Chain Variable Region (VH) comprising the amino acid sequence of SEQ ID NO: 120 and a Light Chain Variable Region (VL) comprising the amino acid sequence of SEQ ID NO: 124; or
l. a Heavy Chain Variable Region (VH) comprising the amino acid sequence of SEQ ID NO: 130 and a Light Chain Variable Region (VL) comprising the amino acid sequence of SEQ ID NO: 134; or
m. a Heavy Chain Variable Region (VH) comprising the amino acid sequence of SEQ ID NO: 140 and a Light Chain Variable Region (VL) comprising the amino acid sequence of SEQ ID NO: 144; or
n. a Heavy Chain Variable Region (VH) comprising the amino acid sequence of SEQ ID NO: 150 and a Light Chain Variable Region (VL) comprising the amino acid sequence of SEQ ID NO: 154; or
o. a Heavy Chain Variable Region (VH) comprising the amino acid sequence of SEQ ID NO: 160 and a Light Chain Variable Region (VL) comprising the amino acid sequence of SEQ ID NO: 164; or
p. a Heavy Chain Variable Region (VH) comprising the amino acid sequence of SEQ ID NO: 170 and a Light Chain Variable Region (VL) comprising the amino acid sequence of SEQ ID NO: 174; or
q. a Heavy Chain Variable Region (VH) comprising the amino acid sequence of SEQ ID NO: 180 and a Light Chain Variable Region (VL) comprising the amino acid sequence of SEQ ID NO: 184; or
r. a Heavy Chain Variable Region (VH) comprising the amino acid sequence of SEQ ID NO: 190 and a Light Chain Variable Region (VL) comprising the amino acid sequence of SEQ ID NO: 194; or
s. a Heavy Chain Variable Region (VH) comprising the amino acid sequence of SEQ ID NO: 200 and a Light Chain Variable Region (VL) comprising the amino acid sequence of SEQ ID NO: 204; or
t. a Heavy Chain Variable Region (VH) comprising the amino acid sequence of SEQ ID NO: 210 and a Light Chain Variable Region (VL) comprising the amino acid sequence of SEQ ID NO: 214; or
u. a Heavy Chain Variable Region (VH) comprising the amino acid sequence of SEQ ID NO: 220 and a Light Chain Variable Region (VL) comprising the amino acid sequence of SEQ ID NO: 224; or
v. a Heavy Chain Variable Region (VH) comprising the amino acid sequence of SEQ ID NO: 230 and a Light Chain Variable Region (VL) comprising the amino acid sequence of SEQ ID NO: 234; or
w. a Heavy Chain Variable Region (VH) comprising the amino acid sequence of SEQ ID NO: 240 and a Light Chain Variable Region (VL) comprising the amino acid sequence of SEQ ID NO: 244; or
x. a Heavy Chain Variable Region (VH) comprising the amino acid sequence of SEQ ID NO: 250 and a Light Chain Variable Region (VL) comprising the amino acid sequence of SEQ ID NO: 254; or
y. a Heavy Chain Variable Region (VH) comprising the amino acid sequence of SEQ ID NO: 260 and a Light Chain Variable Region (VL) comprising the amino acid sequence of SEQ ID NO: 264; or
z. a Heavy Chain Variable Region (VH) comprising the amino acid sequence of SEQ ID NO: 270 and a Light Chain Variable Region (VL) comprising the amino acid sequence of SEQ ID NO: 274; or
aa. a Heavy Chain Variable Region (VH) comprising the amino acid sequence of SEQ ID NO: 280 and a Light Chain Variable Region (VL) comprising the amino acid sequence of SEQ ID NO: 284; or
bb. a Heavy Chain Variable Region (VH) comprising the amino acid sequence of SEQ ID NO: 290; and a Light Chain Variable Region (VL) comprising the amino acid sequence of SEQ ID NO: 294; or
cc. a Heavy Chain Variable Region (VH) comprising the amino acid sequence of SEQ ID NO: 300 and a Light Chain Variable Region (VL) comprising the amino acid sequence of SEQ ID NO:304; or
dd. a Heavy Chain Variable Region (VH) comprising the amino acid sequence of SEQ ID NO: 310; and a Light Chain Variable Region (VL) comprising the amino acid sequence of SEQ ID NO: 314; or
ee. a Heavy Chain Variable Region (VH) comprising the amino acid sequence of SEQ ID NO: 320 and a Light Chain Variable Region (VL) comprising the amino acid sequence of SEQ ID NO: 324; or
ff. a Heavy Chain Variable Region (VH) comprising the amino acid sequence of SEQ ID NO: 330; and a Light Chain Variable Region (VL) comprising the amino acid sequence of SEQ ID NO: 334; or
gg. a Heavy Chain Variable Region (VH) comprising the amino acid sequence of SEQ ID NO: 340 and a Light Chain Variable Region (VL) comprising the amino acid sequence of SEQ ID NO: 344; or
hh. a Heavy Chain Variable Region (VH) comprising the amino acid sequence of SEQ ID NO: 350 and a Light Chain Variable Region (VL) comprising the amino acid sequence of SEQ ID NO: 354; or
ii. a Heavy Chain Variable Region (VH) comprising the amino acid sequence of SEQ ID NO: 360 and a Light Chain Variable Region (VL) comprising the amino acid sequence of SEQ ID NO: 364; or
jj. a Heavy Chain Variable Region (VH) comprising the amino acid sequence of SEQ ID NO: 370 and a Light Chain Variable Region (VL) comprising the amino acid sequence of SEQ ID NO: 374; or
kk. a Heavy Chain Variable Region (VH) comprising the amino acid sequence of SEQ ID NO: 380 and a Light Chain Variable Region (VL) comprising the amino acid sequence of SEQ ID NO: 384; or
ll. a Heavy Chain Variable Region (VH) comprising the amino acid sequence of SEQ ID NO: 390 and a Light Chain Variable Region (VL) comprising the amino acid sequence of SEQ ID NO: 394.

101. The ASC binding molecule of claim 83, comprising:
a) a VH-CDR1 comprising the amino acid sequence of SEQ ID NO: 201, a VH-CDR2 comprising the amino acid sequence of SEQ ID NO: 202, a VH-CDR3 comprising the amino acid sequence SEQ ID NO: 203, a VL-CDR1 comprising the amino sequence SEQ ID NO: 205, a VL-CDR2 comprising the amino acid sequence of SEQ ID NO: 206, and a VL-CDR3 comprising the amino acid sequence of SEQ ID NO: 207; or
b) a Heavy Chain Variable Region (VH) comprising the amino acid sequence of SEQ ID NO: 200 or a Heavy Chain Variable Region (VH) having at least 93%, 94%, 95%, 96%, 97%, 98% or 99% sequence identity to the amino acid sequence of SEQ ID NO: 200; and a Light Chain Variable Region (VL) comprising the amino acid sequence of SEQ ID NO: 204 or a Light Chain Variable Region (VL) having at least 99% sequence identity to the amino acid sequence of SEQ ID NO: 204; or
c) a Heavy Chain Variable Region (VH) comprising the amino acid sequence of SEQ ID NO: 200 and a Light Chain Variable Region (VL) comprising the amino acid sequence of SEQ ID NO: 204.

102. The ASC binding molecule of claim 83, wherein the binding molecule is:
a) an anti-ASC antibody or an antigen-binding fragment thereof; or
b) a heterohybrid anti-ASC antibody or an antigen binding fragment thereof, optionally a humanized antibody or antigen binding fragment thereof; or
c) a monoclonal antibody or an antigen-binding fragment thereof; or
d) an IgA, IgD, IgE, IgM, IgG1, IgG2, IgG2a, IgG2b, IgG3 or IgG4 antibody or antigen-binding fragment thereof, preferably human IgA, IgD, IgE, IgM, IgG1, IgG2, IgG2a, IgG2b, IgG3 or IgG4.

103. An immunoconjugate comprising the ASC binding molecule according to claim 83.

104. A method of human or veterinary therapy comprising administering the ASC binding molecule of claim 83 to a subject.

105. The method of claim 104, comprising the prevention, alleviation, treatment or postponing the onset of a disease, disorder or condition associated with accumulation of ASC or ASC specks, preferably extracellular ASC specks.

106. The method of claim 104 comprising the prevention, alleviation or treatment of a disease, disorder or condition associated with demyelination.

107. The method of claim 105, wherein the disease, disorder or condition associated with accumulation of ASC or ASC specks, preferably ASC specks, more preferably extracellular ASC specks, is selected from either:
a) a central nervous system disease, preferably Parkinson's disease, Alzheimer's disease, multiple sclerosis, amyotrophic lateral sclerosis, traumatic brain injury, spinal cord injury, chronic traumatic encephalopathy; or
b) a Peripheral inflammatory condition, preferably Non-Alcoholic SteatoHepatitis (NASH), Cryopyrin-associated periodic syndrome (CAPS), Chronic obstructive pulmonary disease (COPD), gout, acne, Hidradenitis Suppurativa (HS), Inflammatory Bowel Disease (IBD), Edema (DME), Geographic Atrophy (GA), Coronavirus-associated respiratory distress syndrome (CARDS), Sjögren's Syndrome.

108. A method of preventing or reducing demyelination in a subject, comprising administering the ASC binding molecule of claim 83 to the subject, optionally wherein prevention or reduction of demyelination is improving demyelination score in vivo.

109. A method of reducing levels of:
a) reactive microglia in a subject; or
b) ASC or cleaved capase-1 protein in a subject; or
c) infiltrating CD4+ T-cells in the spinal cord of a subject;
the method comprising administering the ASC binding molecule of claim 83 to the subject.

110. A method for diagnosing a disease, disorder or condition associated with ASC-dependent inflammation comprising the ASC binding molecule of claim 83.

111. A method of:
a) detecting non-polymerized ASC or ASC specks in a sample obtained from a subject, the method comprising contacting the sample with the ASC binding molecule of claim 83 and detecting binding of the ASC binding molecule to non-polymerized ASC or ASC specks in the sample; or
b) quantifying non-polymerized ASC or ASC specks in a sample obtained from a subject, the method comprising contacting the sample with the ASC binding molecule and quantifying non-polymerized ASC or ASC specks in a sample based on the level of binding of the ASC binding molecule to non-polymerized ASC or ASC specks.

112. A method for diagnosing a disease, disorder or condition associated with ASC-dependent inflammation comprising performing the method of claim 111 b), wherein higher levels of non-polymerized ASC or ASC specks in the sample compared with a control level based on healthy subjects are indicative of a disease, disorder or condition associated with ASC-dependent inflammation.

113. A diagnostic composition comprising the ASC binding molecule of claim 83 and an acceptable carrier or excipient, or a pharmaceutic composition comprising the ASC binding molecule and a pharmaceutically acceptable carrier or excipient.

114. A nucleic acid encoding the ASC binding molecule of claim 83, comprising a nucleotide sequence as provided in SEQ ID NO: 18, SEQ ID NO: 19, SEQ ID NO: 28, SEQ ID NO: 29, SEQ ID NO: 38, SEQ ID NO: 39, SEQ ID NO: 48, SEQ ID NO: 49, SEQ ID NO: 58, SEQ ID NO: 59, SEQ ID NO: 68, SEQ ID NO: 69, SEQ ID NO: 78, SEQ ID NO: 79, SEQ ID NO: 88, SEQ ID NO: 89, SEQ ID NO: 98, SEQ ID NO: 99, SEQ ID NO: 118, SEQ ID NO: 119, SEQ ID NO: 128, SEQ ID NO: 129, SEQ ID NO: 138, SEQ ID NO: 139, SEQ ID NO: 148, SEQ ID NO: 149, SEQ ID NO: 158, SEQ ID NO: 159, SEQ ID NO: 168, SEQ ID NO: 169, SEQ ID NO: 178, SEQ ID NO: 179, SEQ ID NO: 188, SEQ ID NO: 189, SEQ ID NO: 198, SEQ ID NO: 199, SEQ ID NO: 208, SEQ ID NO: 209, SEQ ID NO: 218, SEQ ID NO: 219, SEQ ID NO: 228, SEQ ID NO: 229, SEQ ID NO: 238, SEQ ID NO: 239, SEQ ID NO: 248, SEQ ID NO: 249, SEQ ID NO: 258, SEQ ID NO: 259, SEQ ID NO: 268, SEQ ID NO: 269, SEQ ID NO: 278, SEQ ID NO: 279, SEQ ID NO: 288, SEQ ID NO: 289, SEQ ID NO: 298, SEQ ID NO: 299, SEQ ID NO: 308, SEQ ID NO: 309, SEQ ID NO: 318, SEQ ID NO: 319, SEQ ID NO: 328, SEQ ID NO: 329, SEQ ID NO: 338, SEQ ID NO: 339, SEQ ID NO: 348, SEQ ID NO: 349, SEQ ID NO: 358, SEQ ID NO: 359, SEQ ID NO: 368, SEQ ID NO: 369, SEQ ID NO: 378, SEQ ID NO: 379, SEQ ID NO: 388, SEQ ID NO: 389, SEQ ID NO: 398, SEQ ID NO: 399, SEQ ID NO: 408, SEQ ID NO: 409, SEQ ID NO: 418, SEQ ID NO: 419, SEQ ID NO: 428, SEQ ID NO: 429, SEQ ID NO: 438, SEQ ID NO: 439, SEQ ID NO: 448, SEQ ID NO: 458, SEQ ID NO: 468, SEQ ID NO: 478, SEQ ID NO: 488, SEQ ID NO: 498, SEQ ID NO: 508, SEQ ID NO: 518, SEQ ID NO: 528, SEQ ID NO: 538, SEQ ID NO: 548, SEQ ID NO: 558 and 559.

115. A recombinant vector comprising the nucleic acid of claim 114.

116. A host cell comprising the nucleic acid of claim 114.

117. A method for producing an ASC binding molecule, comprising the steps of:
a. culturing the host cell of claim 116 under conditions suitable for producing the ASC binding molecule, and
b. recovering the ASC binding molecule.

118. A kit for diagnosis of a disease, disorder or condition associated with ASC-dependent inflammation, comprising the ASC binding molecule of claim 83 and a container.


SEQUENCE LISTING (559 sequences)

SEQ ID NO: 1 - protein, 195 aa, Homo sapiens
MGRARDAILDALENLTAEELKKFKLKLLSVPLREGYGRIPRGALLSMDALDLTDKLVSFYLETYGAELTANVLRDMGLQEMAGQLQAATHQGSGAAPAGIQAPPQSAAKPGLHFIDQHRAALIARVTNVEWLLDALYGKVLTDEQYQAVRAEPTNPSKMRKLFSFTPAWNWTCKDLLLQALRESQSYLVEDLERS

SEQ ID NO: 2 - protein, 193 aa, Mus musculus
MGRARDAILDALENLSGDELKKFKMKLLTVQLREGYGRIPRGALLQMDAIDLTDKLVSYYLESYGLELTMTVLRDMGLQELAEQLQTTKEESGAVAAAASVPAQSTARTGHFVDQHRQALIARVTEVDGVLDALHGSVLTEGQYQAVRAETTSQDKMRKLFSFVPSWNLTCKDSLLQALKEIHPYLVMDLEQS

SEQ ID NO: 3-9 - skipped (7 entries)

SEQ ID NO: 10 - protein, 111 aa, synthetic construct
QVQLKQSGPGLVQPSQSLSITCTVSGISLSSYGLHWVRQSPGKGLEWLGVIWSGGNTDYSATFISRLSISKDNSKSQVFFKMDSLQVDDTAIYYCARNEVWGQGTSVTVSS

SEQ ID NO: 11 - protein, 5 aa, synthetic construct
SYGLH

SEQ ID NO: 12 - protein, 16 aa, synthetic construct
VIWSGGNTDYSATFIS

SEQ ID NO: 13 - skipped

SEQ ID NO: 14 - protein, 111 aa, synthetic construct
DIVLTQSPASLAVSLGQRATISCRASESVDNYGISFINWFQQKPGQPPKLIIYATSNQGSGVPARFSGSGSGTDFSLNIHPMEEDDTAMYFCQQSKEVPWTFGGGTKLEIK

SEQ ID NO: 15 - protein, 15 aa, synthetic construct
RASESVDNYGISFIN

SEQ ID NO: 16 - protein, 7 aa, synthetic construct
ATSNQGS

SEQ ID NO: 17 - protein, 9 aa, synthetic construct
QQSKEVPWT

SEQ ID NO: 18 - DNA, 333 bp, synthetic construct
  source 1..333: mol_type = other DNA
caggtgcagctgaagcagtcaggacctggcctagtgcagccctcacagagcctgtccatcacctgcacagtctccggcatctcattatctagctatggtctacattgggttcgccagtctccaggaaagggtctggagtggctgggagtgatatggagtggtggaaacacagactatagtgcaactttcatatccagactgagcatcagcaaggacaattccaagagccaagttttctttaaaatggacagtctgcaagttgatgacacagccatatattactgtgccagaaatgaggtctggggtcaaggaacctcagtcaccgtctcttca

SEQ ID NO: 19 - DNA, 333 bp, synthetic construct
  source 1..333: mol_type = other DNA
gacattgtgctgacccaatctccagcttctttggctgtgtctctagggcagagggccaccatctcctgcagagccagcgaaagtgttgataattatggcataagttttataaactggttccaacagaaaccaggacagccacccaaactcatcatctatgctacatccaatcaaggatccggggtccctgccagatttagtggcagtgggtctgggacagacttcagcctcaacatccatcctatggaggaggatgatactgcaatgtatttctgtcaacaaagtaaggaggttccgtggacgttcggtggaggcaccaagctggaaatcaaa

SEQ ID NO: 20 - protein, 118 aa, synthetic construct
QVQLQQSGAELARPGASVKMSCKASGYTFTTYTMHWVKQRPGQGLEWIGYINPSSGYTYYNQNFKDKATLTADKSSSTAYMQLSNLASDDSAVYYCALGGNPAWFAYWGQGTLVTVSA

SEQ ID NO: 21 - protein, 5 aa, synthetic construct
TYTMH

SEQ ID NO: 22 - protein, 17 aa, synthetic construct
YINPSSGYTYYNQNFKD

SEQ ID NO: 23 - protein, 9 aa, synthetic construct
GGNPAWFAY

SEQ ID NO: 24 - protein, 107 aa, synthetic construct
DIVLTQSPATLSVTPGDSVSLSCRASQSIGNNLHWYQHKSHESPRLLIKYASQSISGIPSRFSGSESGTDFTLSINSVETEDFGIYFCQQSKSWPWTFGGGTKLEIK

SEQ ID NO: 25 - protein, 11 aa, synthetic construct
RASQSIGNNLH

SEQ ID NO: 26 - protein, 7 aa, synthetic construct
YASQSIS

SEQ ID NO: 27 - protein, 9 aa, synthetic construct
QQSKSWPWT

SEQ ID NO: 28 - DNA, 354 bp, synthetic construct
  source 1..354: mol_type = other DNA
caggtccagctgcagcagtctggggctgaactggcaagacctggggcctcagtgaagatgtcctgcaaggcttctggctacacctttactacctacacgatgcactgggtaaaacagaggcctggacagggtctggaatggattggatacattaatcctagcagtggttatacttattacaatcagaacttcaaggacaaggccacattgactgcagacaaatcctccagcacagcctacatgcaactgagcaacctggcatctgacgactctgcagtctattactgtgctctagggggtaacccggcctggtttgcttactggggccaggggactctggtcactgtctctgca

SEQ ID NO: 29 - DNA, 321 bp, synthetic construct
  source 1..321: mol_type = other DNA
gatattgtgctaactcagtctccagccaccctgtctgtgactccaggagatagcgtcagtctttcctgcagggccagccaaagtattggcaacaacctacactggtatcaacataaatcacatgagtctccaaggcttctcatcaagtatgcttcccagtccatctctgggatcccctccaggttcagtggcagtgaatcagggacagatttcactctcagtatcaacagtgtggagactgaagattttggaatctatttctgtcaacagagtaagagctggccgtggacgttcggtggaggcaccaagctggaaatcaaa

SEQ ID NO: 30 - protein, 119 aa, synthetic construct
QVQLKQSGPGLVQPSQSLSITCKVSDFSLSHYGVHWVRQSPGKGLEWLGVIWSGGTTEYNEAFISRLTISKDNSKSQILFRMNSLQPADTAIYYCARKGDFYGDYFDYWGQGTTLTVSS

SEQ ID NO: 31 - protein, 5 aa, synthetic construct
HYGVH

SEQ ID NO: 32 - protein, 16 aa, synthetic construct
VIWSGGTTEYNEAFIS

SEQ ID NO: 33 - protein, 11 aa, synthetic construct
KGDFYGDYFDY

SEQ ID NO: 34 - protein, 107 aa, synthetic construct
DIQMTQTSSSLSASLGDRVTISCRASQDISNYLNWYQQRPDGTVKLLIYYTSKVHSGVPSRFSGSGSGTDFSLTISNLEQEDFATYYCQQGNTRPFTFGGGTKLEIK

SEQ ID NO: 35 - protein, 11 aa, synthetic construct
RASQDISNYLN

SEQ ID NO: 36 - protein, 7 aa, synthetic construct
YTSKVHS

SEQ ID NO: 37 - protein, 9 aa, synthetic construct
QQGNTRPFT

SEQ ID NO: 38 - DNA, 357 bp, synthetic construct
  source 1..357: mol_type = other DNA
caggtgcagctgaagcagtcaggacctggcctagtgcagccctcacagagcctgtccatcacctgcaaagtctctgacttctcattaagtcactatggtgtacattgggttcgccagtctccaggaaagggtctggagtggctgggagtgatatggagtggtggaaccacagagtataatgaagctttcatttccagactgaccatcagcaaggacaattccaagagccaaattctctttagaatgaacagtctgcaacctgctgacacagccatatattactgtgccagaaaaggggatttctacggtgactattttgactactggggccaaggtaccactctcacagtctcctca

SEQ ID NO: 39 - DNA, 321 bp, synthetic construct
  source 1..321: mol_type = other DNA
gatatccagatgacacagacttcatcctccctgtctgcctctctgggagacagagtcaccatcagttgcagggcaagtcaggacattagcaattatttaaactggtatcagcagagaccggatggaactgttaaactcctgatctactacacatcgaaggtgcactcaggagtcccatcaaggttcagtggcagtgggtctggaacagatttttctctcaccattagcaacctggagcaagaagattttgccacttactattgccaacagggtaatacgcgtccgttcacgttcggaggggggaccaagctggaaataaaa

SEQ ID NO: 40 - protein, 117 aa, synthetic construct
EVQLQQSGADLVKPGASVKLSCTASGFNIEDTYIHWVKQRPARGLEWVGRIDPANGNTDYDPKFQGKATMITDTSSNTVYLQVSSLTSEDTAVYFCGRHDGYDMDYWGQGTSVTVSS

SEQ ID NO: 41 - protein, 5 aa, synthetic construct
DTYIH

SEQ ID NO: 42 - protein, 17 aa, synthetic construct
RIDPANGNTDYDPKFQG

SEQ ID NO: 43 - protein, 8 aa, synthetic construct
HDGYDMDY

SEQ ID NO: 44 - protein, 112 aa, synthetic construct
DIVMSQSPSSLAVSAGEKVTMSCQSSQSLLNGRTRRNYLAWYQQKPGQSPKLLIYWASTRKSGVPDRFTGSGSGTDFTLTITSVQAEDLAVYYCKQSYNLVTFGAGTKLELK

SEQ ID NO: 45 - protein, 17 aa, synthetic construct
QSSQSLLNGRTRRNYLA

SEQ ID NO: 46 - protein, 7 aa, synthetic construct
WASTRKS

SEQ ID NO: 47 - protein, 8 aa, synthetic construct
KQSYNLVT

SEQ ID NO: 48 - DNA, 351 bp, synthetic construct
  source 1..351: mol_type = other DNA
gaggttcagctgcagcagtctggggcagaccttgtgaagccaggggcctcagtcaaattgtcctgcacagcttctggcttcaacattgaagacacctatatccactgggtgaaacagaggcctgcacggggcctggagtgggttggaaggattgatcctgcgaatggtaatactgattatgacccgaagttccagggcaaggccactatgataacagacacatcctccaacacagtctacctgcaggtcagcagcctgacatctgaggacactgccgtctatttctgtggtagacacgacgggtatgatatggactactggggtcaaggaacctcagtcaccgtctcctca

SEQ ID NO: 49 - DNA, 336 bp, synthetic construct
  source 1..336: mol_type = other DNA
gacattgtgatgtcacagtctccttcctccctggctgtgtcagcaggagagaaggtcactatgagctgccaatccagtcagagtctactcaacggtagaacccggaggaactacttggcttggtaccagcagaaaccagggcagtctcctaaactgctgatctactgggcatccactcggaaatctggggtccctgatcgcttcacaggcagtggatctgggacagatttcactctcaccatcaccagtgtgcaggctgaagacctggcagtttattactgcaagcaatcttataatctggtcacgttcggtgctgggaccaagctggaactgaaa

SEQ ID NO: 50 - protein, 121 aa, synthetic construct
QVQLQQPGAELVRPGASVILSCKASGYTFTSSWITWVKQRPGQGLEWIGNIYPSDSYTNSNQKFKDKATLTVDKSSSTAYMHLISPTSEDSAVYYCTRKDFVYDAWYFDVWGAGTTVTVSS

SEQ ID NO: 51 - protein, 5 aa, synthetic construct
SSWIT

SEQ ID NO: 52 - protein, 17 aa, synthetic construct
NIYPSDSYTNSNQKFKD

SEQ ID NO: 53 - protein, 12 aa, synthetic construct
KDFVYDAWYFDV

SEQ ID NO: 54 - protein, 107 aa, synthetic construct
DIVMTQSQKFMSTSVGDRVTVTCKASQNVGTNVAWYQQRPGQSPKPLIYSASYRNSGVPDRFTGSGSGTDFTLTITNVQSEDLAEYFCQQYNNYPLTFGAGTKLELK

SEQ ID NO: 55 - protein, 11 aa, synthetic construct
KASQNVGTNVA

SEQ ID NO: 56 - protein, 7 aa, synthetic construct
SASYRNS

SEQ ID NO: 57 - protein, 9 aa, synthetic construct
QQYNNYPLT

SEQ ID NO: 58 - DNA, 363 bp, synthetic construct
  source 1..363: mol_type = other DNA
caggtccaactgcagcagcctggggctgagctggtgaggcctggggcttcagtgatactgtcctgcaaggcttctggctacaccttcaccagctcctggattacctgggtgaaacagaggcctggacaaggccttgagtggatcgggaatatttatccttctgatagttatactaactccaatcaaaagttcaaggacaaggccacattgactgtagacaaatcttccagcacagcctacatgcacctcatcagcccgacatctgaggactctgcagtctattactgtacaagaaaagactttgtttacgacgcctggtacttcgatgtctggggcgcagggaccacggtcaccgtctcctca

SEQ ID NO: 59 - DNA, 321 bp, synthetic construct
  source 1..321: mol_type = other DNA
gacattgtgatgacccagtctcaaaaattcatgtccacatcagtaggagacagggtcaccgtcacctgcaaggccagtcagaatgtgggtactaatgtagcctggtatcaacagagaccagggcaatctcctaaaccactgatttactcggcatcctaccggaacagtggagtccctgatcgcttcacaggcagtggatctgggacagatttcactctcaccatcaccaatgtgcagtctgaagacttggcagagtatttctgtcagcaatataacaactatcctctcacgttcggtgctgggaccaagctggagctgaaa

SEQ ID NO: 60 - protein, 120 aa, unidentified
QVQLKESGPGLVAPSQSLSITCTVSGFSLTSYGVHWVRQPPGKGLEWLGVIWAGGSTNYNSALMSRLSISKDNSKSQVFLKMNSLQTADTAMYYCARWDYVNSYEAMDYWGQGTSVTVSS

SEQ ID NO: 61 - protein, 5 aa, synthetic construct
SYGVH

SEQ ID NO: 62 - protein, 16 aa, synthetic construct
VIWAGGSTNYNSALMS

SEQ ID NO: 63 - protein, 12 aa, synthetic construct
WDYVNSYEAMDY

SEQ ID NO: 64 - protein, 112 aa, synthetic construct
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPYTFGGGTKLEIK

SEQ ID NO: 65 - protein, 16 aa, synthetic construct
RSSQSLVHSNGNTYLH

SEQ ID NO: 66 - protein, 7 aa, synthetic construct
KVSNRFS

SEQ ID NO: 67 - protein, 9 aa, synthetic construct
SQSTHVPYT

SEQ ID NO: 68 - DNA, 360 bp, synthetic construct
  source 1..360: mol_type = other DNA
caggtgcagctgaaggagtcaggacctggcctggtggcgccctcacagagcctgtccatcacttgcactgtctctgggttttcattaaccagttatggtgtacactgggttcgccagcctccaggaaagggtctggagtggctgggagtaatatgggctggtggaagcacaaattataattcggctctcatgtccagactgagcatcagcaaagacaactccaagagccaagttttcttaaaaatgaacagtctgcaaactgctgacacagccatgtactactgtgccagatgggactacgttaatagctacgaggctatggactactggggtcaaggaacctcagtcaccgtctcctca

SEQ ID NO: 69 - DNA, 336 bp, synthetic construct
  source 1..336: mol_type = other DNA
gatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtacacgttcggaggggggaccaagctggaaataaaa

SEQ ID NO: 70 - protein, 114 aa, synthetic construct
EVQLVESGGGFVKPGGSLKLSCAASGFTFSNYAMSWVRQTPEKRLEWVATIYSGGIFTYYPDSVKGRFTISRDSAKNTLYLQMTSLRSEDTAIYYCARLGGSYWGQGTLVTVSA

SEQ ID NO: 71 - protein, 5 aa, synthetic construct
NYAMS

SEQ ID NO: 72 - protein, 17 aa, synthetic construct
TIYSGGIFTYYPDSVKG

SEQ ID NO: 73 - protein, 5 aa, synthetic construct
LGGSY

SEQ ID NO: 74 - protein, 109 aa, synthetic construct
QAVVTQESALTTSPGETVTLTCRSSTGAVTTNNYANWVQEKPDHFFTGLIGGTDNRPPGVPARFSGSLIGDKAALTITGAQTEDEAIYFCALWYNNHWVFGGGTKLTVL

SEQ ID NO: 75 - protein, 14 aa, synthetic construct
RSSTGAVTTNNYAN

SEQ ID NO: 76 - protein, 7 aa, synthetic construct
GTDNRPP

SEQ ID NO: 77 - protein, 9 aa, synthetic construct
ALWYNNHWV

SEQ ID NO: 78 - DNA, 342 bp, synthetic construct
  source 1..342: mol_type = other DNA
gaagtgcagctggtggagtctgggggaggcttcgtgaagcctggagggtccctgaaactctcctgtgcagcctctggattcactttcagtaactatgccatgtcttgggttcgccagactccggagaagaggctggaatgggtcgcaaccatttatagtggtggtatattcacctactatccagacagtgtgaagggtcgattcaccatctccagagacagtgccaagaacaccctgtacctgcaaatgaccagtctgaggtctgaggacacggccatatattactgtgcaagacttgggggttcttactggggccaagggactctggtcactgtctctgca

SEQ ID NO: 79 - DNA, 327 bp, synthetic construct
  source 1..327: mol_type = other DNA
caggctgttgtgactcaggaatctgcactcaccacatcacctggtgaaacagtcacactcacttgtcgctcaagtactggggctgttacaactaataactatgccaactgggtccaagaaaaaccagatcattttttcactggtctaataggtggtaccgacaaccgacctccaggtgttcctgccagattctcaggctccctgattggagacaaggctgccctcaccatcacaggggcacagactgaggatgaggcaatatatttctgtgctctatggtacaacaaccattgggtgttcggtggaggaaccaaactgactgtccta

SEQ ID NO: 80 - protein, 117 aa, synthetic construct
EVKLEESGGGLVHPGGSMKLSCVASGFTFSNYWMNWVRQSPEKGLEWVAQIRLKSENYATHSAESVKGRLTISRDDSKSSVYLQMNNLRAEDTGIYYCLAGWGLDYWGQGTTLTVSS

SEQ ID NO: 81 - protein, 5 aa, synthetic construct
NYWMN

SEQ ID NO: 82 - protein, 19 aa, synthetic construct
QIRLKSENYATHSAESVKG

SEQ ID NO: 83 - protein, 6 aa, synthetic construct
GWGLDY

SEQ ID NO: 84 - protein, 112 aa, synthetic construct
DIVMTQAAFSNPVTLGTSASISCRSSKTLLHSDGFTYLYWYLQRPGQSPQLLIHRVSNLASGVPNRFSGSESGTDFTLRISRVEPEDVGVYYCAQMLELPLTFGSGTKLELK

SEQ ID NO: 85 - protein, 16 aa, synthetic construct
RSSKTLLHSDGFTYLY

SEQ ID NO: 86 - protein, 7 aa, synthetic construct
RVSNLAS

SEQ ID NO: 87 - protein, 9 aa, synthetic construct
AQMLELPLT

SEQ ID NO: 88 - DNA, 351 bp, synthetic construct
  source 1..351: mol_type = other DNA
gaagtgaagcttgaggagtctggaggaggcttggtgcatcctggaggatccatgaaactctcctgtgttgcctctggattcactttcagtaactactggatgaactgggtccgccagtctccagagaaggggcttgagtgggttgctcaaattagattgaaatctgaaaattatgcgacacattctgcggagtctgttaaagggaggctcaccatctcaagagatgattccaaaagtagtgtctacctgcaaatgaacaacttgagggctgaagacactggaatttattactgcttagcgggatggggtcttgactactggggccaaggcaccactctcacagtctcctca

SEQ ID NO: 89 - DNA, 336 bp, synthetic construct
  source 1..336: mol_type = other DNA
gatattgtgatgacgcaggctgccttctccaatccagtcacccttggaacatcagcttccatctcctgcaggtctagtaagactctcctacatagtgatggcttcacttatttgtattggtatctgcagaggccaggccagtctcctcaactcctgatacatcgggtgtccaatctggcctcaggagtcccaaacaggttcagcggcagtgagtcaggcactgatttcacactgagaatcagcagagtggagcctgaggatgtgggtgtttattactgtgctcaaatgctagaactcccgctcacgtttggttctgggaccaagctggagctgaag

SEQ ID NO: 90 - protein, 118 aa, synthetic construct
QIQLVQSGPELKKPGETVKISCKASGYTFTKYGMNWVKQAPGKGLKRMGWINTYTGEPTYSDDFKGRFAFSLETSASTAYLQINNLKNDDTATYFCARLGYANSFDSWGQGTTLTVSS

SEQ ID NO: 91 - protein, 5 aa, synthetic construct
KYGMN

SEQ ID NO: 92 - protein, 17 aa, synthetic construct
WINTYTGEPTYSDDFKG

SEQ ID NO: 93 - protein, 9 aa, synthetic construct
LGYANSFDS

SEQ ID NO: 94 - protein, 107 aa, synthetic construct
DIQMTQSPASLSATVGETVTITCRASENIYTYLAWYQQKQGKSPQLLVHNANTLVEGVPSRFSGSGSGTQFSLKINSLEPEDFGNYYCQYHYGSPYTFGGGTKLEIK

SEQ ID NO: 95 - protein, 11 aa, synthetic construct
RASENIYTYLA

SEQ ID NO: 96 - protein, 7 aa, synthetic construct
NANTLVE

SEQ ID NO: 97 - protein, 9 aa, synthetic construct
QYHYGSPYT

SEQ ID NO: 98 - DNA, 354 bp, synthetic construct
  source 1..354: mol_type = other DNA
cagatccagttggtgcagtctggacctgagctgaagaagcctggagagacagtcaagatctcctgcaaggcttctggatataccttcacaaagtatggaatgaactgggtgaagcaggctccaggaaagggtttaaagaggatgggctggataaacacctacactggagagccaacatattctgatgacttcaagggacgatttgccttttctttggaaacctctgccagcactgcctatttgcagatcaacaacctcaaaaatgacgacacggctacatatttctgtgcaagactgggctatgctaattcctttgactcctggggccaaggcaccactctcacagtctcctcc

SEQ ID NO: 99 - DNA, 321 bp, synthetic construct
  source 1..321: mol_type = other DNA
gacatccagatgactcagtctccagcctccctatctgcaactgtgggagaaactgtcaccatcacatgtcgagcaagtgaaaatatttacacttatttagcatggtatcagcagaaacagggaaaatctcctcagctcctggtccataatgcaaacaccttagtagaaggtgtgccatcaaggttcagtggcagtggatcaggcacacagttttctctgaagatcaacagcctagagcctgaagattttgggaattattattgtcaatatcattatggtagtccgtacacgttcggaggggggaccaagctggagataaag

SEQ ID NO: 100-109 - skipped (10 entries)

SEQ ID NO: 110 - protein, 123 aa, synthetic construct
QVQLQQSGAELVRPGTSVKLSCKASGYTFTSYWMHWVKQRPGQGLEWIGGIDPSDSYTKYNQKFKAKATLTADTSSSTAYMQLSSLTSEDSAIFYCAREEASTVVAHWYFDVWGTGTTVTVSS

SEQ ID NO: 111 - protein, 5 aa, synthetic construct
SYWMH

SEQ ID NO: 112 - protein, 17 aa, synthetic construct
GIDPSDSYTKYNQKFKA

SEQ ID NO: 113 - protein, 14 aa, synthetic construct
EEASTVVAHWYFDV

SEQ ID NO: 114 - protein, 107 aa, synthetic construct
DIVLTQSPATLSVTPGDSVSLSCRASQSISNNLHWYQQKSHESPRLLIKYTSQSISGIPSRFSGSGSGTDYTLSINSVETEDFGMYFCQQSNNWPYTFGGGTKLEIK

SEQ ID NO: 115 - protein, 11 aa, synthetic construct
RASQSISNNLH

SEQ ID NO: 116 - protein, 7 aa, synthetic construct
YTSQSIS

SEQ ID NO: 117 - protein, 9 aa, synthetic construct
QQSNNWPYT

SEQ ID NO: 118 - DNA, 369 bp, synthetic construct
  source 1..369: mol_type = other DNA
caggtccaactgcagcagtctggggctgagctggtgaggcctgggacttcagtgaagttgtcctgcaaggcttctggctacaccttcaccagctactggatgcactgggtaaagcagaggcctggacaaggccttgagtggatcggagggattgatccttctgatagttatactaagtacaatcaaaagttcaaggccaaggccactttgactgcagacacatcctccagcacagcctacatgcagctcagcagcctgacatctgaggactctgcgatcttttactgtgcaagagaggaggcctctacggtagtcgcccactggtacttcgatgtctggggcacagggaccacggtcaccgtctcctca

SEQ ID NO: 119 - DNA, 321 bp, synthetic construct
  source 1..321: mol_type = other DNA
gatattgtgctaactcagtctccagccaccctgtctgtgactccaggagatagcgtcagtctttcctgcagggccagccaaagtattagcaacaacctacactggtatcaacaaaaatcacatgagtctccaaggcttctcatcaaatatacttcccagtccatttctgggatcccctccaggttcagtggcagtggatcagggacagattacactctcagcatcaacagtgtggagactgaagattttggaatgtatttctgtcaacagagtaacaactggccgtacacgttcggaggggggaccaagctggaaataaaa

SEQ ID NO: 120 - protein, 113 aa, synthetic construct
EVQLVESGGGLVKPGGSLKLSCAASGFTFSDSYMYWLRQTPEKRLEWVASISDGGRYTYYSDSVKGRFTISRDNVKNNLYLQMTSLRSEDTAIYYCARGLDYWGQGTTLTVSS

SEQ ID NO: 121 - protein, 5 aa, synthetic construct
DSYMY

SEQ ID NO: 122 - protein, 17 aa, synthetic construct
SISDGGRYTYYSDSVKG

SEQ ID NO: 123 - protein, 4 aa, synthetic construct
GLDY

SEQ ID NO: 124 - protein, 107 aa, synthetic construct
DIKMTQSPSSMYASLGERVTITCKASQDINSYLSWFQQKPGKSPKTLIYRANRLVDGVPSRFSGSGSGQDYSLTISSLEYEDMGIYYCLQYAESTWTFGGGTKLEIK

SEQ ID NO: 125 - protein, 11 aa, synthetic construct
KASQDINSYLS

SEQ ID NO: 126 - protein, 7 aa, synthetic construct
RANRLVD

SEQ ID NO: 127 - protein, 9 aa, synthetic construct
LQYAESTWT

SEQ ID NO: 128 - DNA, 339 bp, synthetic construct
  source 1..339: mol_type = other DNA
gaagtgcagctggtggagtctgggggaggcttagtgaagcctggagggtccctgaaactctcctgtgcagcctctggattcactttcagtgactcttatatgtattggcttcgccagactccggaaaagaggctggagtgggtcgcatccattagtgatggtggtagatacacctattattcagacagtgtgaaggggcgattcaccatctccagagacaatgtcaagaacaacctgtacctgcaaatgaccagtctgaggtctgaggacacagccatctattactgtgcaagaggtcttgactattggggccaaggcaccactctcacagtctcctca

SEQ ID NO: 129 - DNA, 321 bp, synthetic construct
  source 1..321: mol_type = other DNA
gacatcaagatgacccagtctccatcttccatgtatgcatctctaggagagagagtcactatcacttgcaaggcgagtcaggacattaatagctatttaagctggttccagcagaaaccagggaaatctcctaagaccctgatctatcgtgcaaacagattggtagatggggtcccatcaaggttcagtggcagtggatctgggcaagattattctctcaccatcagcagcctggagtatgaagatatgggaatttattattgtctacagtatgctgagtccacgtggacgttcggtggaggcaccaagctggaaatcaaa

SEQ ID NO: 130 - protein, 122 aa, synthetic construct
QVQVQQSGADLARPGASVKLSCKASGYTFTNYGINWVKQRTGQGLEWIGEIYPKNIDIYYNEKFKGKATLTADKSSSTAYMELRSLTSEDSAVYFCGRSSFGFSGNYAMDYWGQGTSVTVSS

SEQ ID NO: 131 - protein, 5 aa, synthetic construct
NYGIN

SEQ ID NO: 132 - protein, 17 aa, synthetic construct
EIYPKNIDIYYNEKFKG

SEQ ID NO: 133 - protein, 13 aa, synthetic construct
SSFGFSGNYAMDY

SEQ ID NO: 134 - protein, 112 aa, synthetic construct
DVVMTQTPLSLPVSLGDQASISCRSSQNLIHSNGITYLHWYLQKPGQSPKLLIYKVSNRFSGVPHRFSGSGSGTDFTLKISRVEAEDLGVYFCSQSTHVPWTFGGGTKLEIK

SEQ ID NO: 135 - protein, 16 aa, synthetic construct
RSSQNLIHSNGITYLH

SEQ ID NO: 136 - skipped

SEQ ID NO: 137 - protein, 9 aa, synthetic construct
SQSTHVPWT

SEQ ID NO: 138 - DNA, 366 bp, synthetic construct
  source 1..366: mol_type = other DNA
caggttcaggtgcagcagtctggagctgacctggcgaggcctggggcttcagtgaaactgtcctgcaaggcttctggctacaccttcacaaactatggtataaactgggtgaagcagagaactggacagggccttgagtggattggggagatttatcctaaaaatattgatatttactacaatgagaaattcaagggcaaggccacactgactgcagacaaatcctccagcacagcgtacatggagctccgcagcctgacatctgaggactctgcggtctatttctgtggaagatcaagctttggttttagcgggaactatgctatggactattggggtcaaggaacctcagtcaccgtctcctca

SEQ ID NO: 139 - DNA, 336 bp, synthetic construct
  source 1..336: mol_type = other DNA
gatgttgtgatgacccagactccactctccctgcctgtcagtcttggagatcaagcctccatctcttgcagatctagtcagaaccttatacacagtaatggaatcacctatttacattggtacctgcagaagccaggccagtctccaaagctcctgatctacaaagtttccaaccgattttctggggtcccacacaggttcagtggcagtggatcagggacagatttcacactcaagatcagcagagtggaggctgaggatctgggagtttatttctgctctcaaagtacacatgttccgtggacgttcggtggaggcaccaagctggaaatcaaa

SEQ ID NO: 140 - protein, 118 aa, synthetic construct
QVQLQQSGAELAKPGASVKLSCKASGYTFTSYWMHWVKQRPGQGLEWIGYINPSSGYTKCNQKFKDKATLTADKSSSTAYMQLSSLTYEDSAVYYCVFPFDYDGFDYWGQGTTLTVSS

SEQ ID NO: 141 - skipped

SEQ ID NO: 142 - protein, 17 aa, synthetic construct
YINPSSGYTKCNQKFKD

SEQ ID NO: 143 - protein, 9 aa, synthetic construct
PFDYDGFDY

SEQ ID NO: 144 - protein, 112 aa, synthetic construct
DVLMTQTPLSLPVSLGDQASISCRSSQSIVHSNGNTYLEWYLQKPGQSPKLLIYKVSNRFSGVPDRFSGSGSGTVFTLKISRVEAEDLGVYYCFQDSHFPFTFGSGTKLEIK

SEQ ID NO: 145 - protein, 16 aa, synthetic construct
RSSQSIVHSNGNTYLE

SEQ ID NO: 146 - skipped

SEQ ID NO: 147 - protein, 9 aa, synthetic construct
FQDSHFPFT

SEQ ID NO: 148 - DNA, 354 bp, synthetic construct
  source 1..354: mol_type = other DNA
caggtccagctgcagcagtctggggctgaactggcaaaacctggggcctcagtgaagctgtcctgcaaggcttctggctacacctttactagctactggatgcactgggtaaaacagaggcctggacagggtctggaatggattggatacattaatcctagcagtggttatactaagtgcaatcagaagttcaaggacaaggccacattgactgcagacaaatcctccagcacagcctacatgcagctgagcagcctgacatatgaggactctgcagtctattactgcgtgttcccatttgattacgacggctttgactactggggccaaggcaccactctcacagtctcctca

SEQ ID NO: 149 - DNA, 336 bp, synthetic construct
  source 1..336: mol_type = other DNA
gatgttttgatgacccaaactccactctccctgcctgtcagtcttggagatcaagcctccatctcttgcagatctagtcagagcattgtccatagtaatggaaacacctatttagaatggtacctgcagaaaccaggccagtctccaaagctcctgatctacaaagtttccaaccgattttctggggtcccagacaggttcagtggcagtggatcagggacagttttcactctcaagatcagcagagtggaggctgaggatctgggagtttattactgctttcaagattcacattttccattcacgttcggctcggggacaaagttggaaataaaa

SEQ ID NO: 150 - protein, 118 aa, synthetic construct
EVQLQQSGAELVRPGSSVKMSCETSGYTFTTYGLTWVKQRPGQGLEWIGYIYVGNGYTEYNEKFKDKATLTSDTSSSIVYMQLSSLTSEDSAIYFCARLGLRRHFDYWGQGTTLTVSS

SEQ ID NO: 151 - protein, 5 aa, synthetic construct
TYGLT

SEQ ID NO: 152 - protein, 17 aa, synthetic construct
YIYVGNGYTEYNEKFKD

SEQ ID NO: 153 - protein, 9 aa, synthetic construct
LGLRRHFDY

SEQ ID NO: 154 - protein, 107 aa, synthetic construct
AIQMTQTTSSLSASLGDRVTISCRASQDISNYLHWYQQKPDGTVKLLIFFTSGLRSGVPSRFSGSGSGTDYSLTISNLEQEDIATYFCQQSILLPWTFGGGTKLEIK

SEQ ID NO: 155 - protein, 11 aa, synthetic construct
RASQDISNYLH

SEQ ID NO: 156 - protein, 7 aa, synthetic construct
FTSGLRS

SEQ ID NO: 157 - protein, 9 aa, synthetic construct
QQSILLPWT

SEQ ID NO: 158 - DNA, 354 bp, synthetic construct
  source 1..354: mol_type = other DNA
gaggtccagctgcagcagtctggagctgagctggtgaggcctgggtcctcagtgaagatgtcctgcgagacttctggatatacattcacaacctacggtttgacctgggtgaagcagaggcctggacagggcctggaatggattggatatatttatgttggaaatggttatactgagtataatgagaagttcaaggacaaggccacactgacttctgacacatcttccagtatagtctatatgcagctcagcagcctgacatctgaggactctgcaatctatttctgtgcaagattgggattacgtcgtcattttgactactggggccaaggcaccactctcacagtctcctca

SEQ ID NO: 159 - DNA, 321 bp, synthetic construct
  source 1..321: mol_type = other DNA
gctatccagatgacccagactacatcctccctgtctgcctcgctaggagacagagtcaccatcagttgcagggcaagtcaggacattagcaattatttacactggtatcagcagaaaccagatggaactgttaaactcctgatcttcttcacatctggattacgctcaggagtcccatcaaggttcagtggcagtgggtctggaacagattattctctcaccattagcaacctggagcaagaagatattgccacttatttttgtcaacagagtattctgcttccgtggacgttcggtggaggcaccaagctggaaatcaaa

SEQ ID NO: 160 - protein, 117 aa, synthetic construct
EVQLQQSGAELVRPGSSVKMSCKTSGFTFTSYGISWVIQRPEQGLEWIGYIYIRNGYTEYNEKFKGKATLTSDTSSNTAYMQLSNLASEDSAIYFCVRIHRRFFDYWGQGTTLTVSS

SEQ ID NO: 161 - protein, 5 aa, synthetic construct
SYGIS

SEQ ID NO: 162 - protein, 17 aa, synthetic construct
YIYIRNGYTEYNEKFKG

SEQ ID NO: 163 - protein, 8 aa, synthetic construct
IHRRFFDY

SEQ ID NO: 164 - protein, 107 aa, synthetic construct
DIQMTQTTSSLSASLGDRVTISCSASQGISNYLNWFQQKPNGTVRLLIYYTSSLHSGVPSRFSGSGSGTDYSLTISNLEPEDIATYYCQQYDRLPWTFGGGTKLEIK

SEQ ID NO: 165 - protein, 11 aa, synthetic construct
SASQGISNYLN

SEQ ID NO: 166 - protein, 7 aa, synthetic construct
YTSSLHS

SEQ ID NO: 167 - protein, 9 aa, synthetic construct
QQYDRLPWT

SEQ ID NO: 168 - DNA, 351 bp, synthetic construct
  source 1..351: mol_type = other DNA
gaggtccagcttcagcagtctggagctgagctggtgaggcctgggtcctcagtgaagatgtcctgtaagacttctggatttacattcacaagttacggtataagttgggtgattcagaggcccgaacagggcctggaatggattggatatatttatattagaaatggttatactgaatataatgagaagtttaagggcaaggccacactgacttcagacacatcctccaacacagcctacatgcagctcagcaacctggcatctgaggactctgcaatctatttttgtgtaagaattcatcgacggttttttgactactggggccaaggcaccactctcacagtctcctca

SEQ ID NO: 169 - DNA, 321 bp, synthetic construct
  source 1..321: mol_type = other DNA
gatatccagatgacacagactacatcctccctgtctgcctctctgggagacagagtcaccatcagttgcagtgcaagtcagggcattagcaattatttaaactggtttcagcagaaaccaaatggaactgttagactcctgatctattacacatcaagtttacactcaggagtcccatcaaggttcagtggcagtgggtctgggacagattattctctcaccatcagcaacctggaacctgaagatattgccacttattattgtcagcaatatgataggcttccgtggacgttcggtggaggcaccaagctggaaatcaag

SEQ ID NO: 170 - protein, 118 aa, synthetic construct
EVQLQQSGAELVRPGSSVKMTCKTSGYSFSSYGMSWVKQRPGQGLEWIGYIYVGNGYTEYNEKFKGKATLTSDTSSSTVYMDFRSLTSEDSAIYFCARLNRGRALDFWGQGTTLTVSS

SEQ ID NO: 171 - protein, 5 aa, synthetic construct
SYGMS

SEQ ID NO: 172 - protein, 17 aa, synthetic construct
YIYVGNGYTEYNEKFKG

SEQ ID NO: 173 - protein, 9 aa, synthetic construct
LNRGRALDF

SEQ ID NO: 174 - protein, 111 aa, synthetic construct
DIVLTQSPASLAVSLGQRATISCRASQSVSTSGHSYMHWYQQKPRQSPKLLIHFASNLESGVPARFTGSGSGTDFTLNIHPVEEEDAATYYCQHSWDLPYTFGGGTKLEIK

SEQ ID NO: 175 - protein, 15 aa, synthetic construct
RASQSVSTSGHSYMH

SEQ ID NO: 176 - protein, 7 aa, synthetic construct
FASNLES

SEQ ID NO: 177 - protein, 9 aa, synthetic construct
QHSWDLPYT

SEQ ID NO: 178 - DNA, 354 bp, synthetic construct
  source 1..354: mol_type = other DNA
gaggtccagcttcagcagtctggagctgagctggtgaggcctgggtcctcagtgaagatgacctgcaagacttctggatattcattctcaagctacggtatgagttgggtgaagcagaggcctggacagggcctggaatggattggatatatttatgttggaaatggttatactgaatacaatgagaagttcaagggcaaggccacactgacttcagacacatcctccagcacagtctacatggactttagaagcctgacatctgaagactctgcaatttatttctgtgcaagattgaataggggtagggcccttgacttctggggccaaggcaccactctcacagtctcttca

SEQ ID NO: 179 - DNA, 333 bp, synthetic construct
  source 1..333: mol_type = other DNA
gacattgtgctgacacagtctcctgcttccttagctgtatctctggggcagagggccaccatctcatgccgggccagccaaagtgtcagtacatctggccacagttatatgcactggtaccaacagaaacccagacagtcacccaaactcctcatccattttgcatccaacctagaatctggggtccctgccaggttcactggcagtgggtctgggacagacttcaccctcaacatccatcctgtggaggaggaggatgctgcaacctattattgtcaacacagttgggaccttccttacacgttcggaggagggaccaagctggaaataaaa

SEQ ID NO: 180 - protein, 114 aa, synthetic construct
QVQLQQSEAELARPGASVKLSCKASGYTFTSYGIFWVKQRTGQGLEWIGEIYPRSGNTYYNENFRDKATLTADKSSSTAYMELRSLTSEDSAVYFCARQRDDYWGQGTTLTVSS

SEQ ID NO: 181 - protein, 5 aa, synthetic construct
SYGIF

SEQ ID NO: 182 - protein, 17 aa, synthetic construct
EIYPRSGNTYYNENFRD

SEQ ID NO: 183 - protein, 5 aa, synthetic construct
QRDDY

SEQ ID NO: 184 - protein, 106 aa, synthetic construct
QIVLTQSPAIMSASPGEKVTITCSVSSSVSYMYWFQQKPGTSPKVWIYSTSNLASGVPARFSGRGSGTSYSLTISRMEAEDAATYYCQQRSSYPFTFGSGTKLEIK

SEQ ID NO: 185 - protein, 10 aa, synthetic construct
SVSSSVSYMY

SEQ ID NO: 186 - protein, 7 aa, synthetic construct
STSNLAS

SEQ ID NO: 187 - protein, 9 aa, synthetic construct
QQRSSYPFT

SEQ ID NO: 188 - DNA, 342 bp, synthetic construct
  source 1..342: mol_type = other DNA
caggttcagctgcagcagtctgaagctgagctggcgaggcctggggcttcagtgaaactgtcctgcaaggcttctggctacaccttcacaagctatggtatattctgggtgaagcagagaactggacagggccttgagtggattggagaaatttatcctagaagtggtaatacttactacaatgagaatttcagggacaaggccacactgactgcagacaaatcctccagcacagcgtacatggagctccgcagcctgacatctgaggactctgcggtctatttctgtgcaagacaacgggatgactactggggccaaggtaccactctcacagtctcctcc

SEQ ID NO: 189 - DNA, 318 bp, synthetic construct
  source 1..318: mol_type = other DNA
caaattgttctcacccagtctccagcaatcatgtctgcatctccaggggagaaggtcaccataacctgcagtgtcagctcaagtgtaagttacatgtactggttccagcagaagccaggcacttctcccaaagtctggatttatagcacatccaacctggcttctggagtccctgctcgcttcagtggcagaggatctgggacctcttactctctcacaatcagccgaatggaggctgaagatgctgccacttattactgccagcaaaggagtagttacccattcacgttcggctcggggacaaagttggaaataaaa

SEQ ID NO: 190 - protein, 124 aa, synthetic construct
QVQLQQSGAELARPGASVKLSCKASGYNFTTYGINWVKQRTGQGLEWIGKIYPRSGNTHNNENFKGKATLTADTSSSTAYMDLRSLTSEDSAVYFCARSPYAFYSSSYWYFDVWGTGTTVTVSS

SEQ ID NO: 191 - protein, 5 aa, synthetic construct
TYGIN

SEQ ID NO: 192 - protein, 17 aa, synthetic construct
KIYPRSGNTHNNENFKG

SEQ ID NO: 193 - protein, 15 aa, synthetic construct
SPYAFYSSSYWYFDV

SEQ ID NO: 194 - protein, 107 aa, synthetic construct
DIKMTQSPSSMYASLGERVTITCKASQDIKSYLSWYQQKPWKSPKTLIYYATSLADGVPSRFSGSGSGQDYSLTISSLESDDTATYYCLQHGESPFTFGSGTKLEIK

SEQ ID NO: 195 - protein, 11 aa, synthetic construct
KASQDIKSYLS

SEQ ID NO: 196 - protein, 7 aa, synthetic construct
YATSLAD

SEQ ID NO: 197 - protein, 9 aa, synthetic construct
LQHGESPFT

SEQ ID NO: 198 - DNA, 372 bp, synthetic construct
  source 1..372: mol_type = other DNA
caggttcagctgcagcagtctggagctgagctggcgaggcctggggcttcagtgaagctgtcctgcaaggcttctggctacaatttcacaacgtatggtataaactgggtgaagcagagaactggacagggcctggagtggattggaaagatttatcctagaagtggtaatactcacaacaacgagaatttcaagggcaaggccacactgactgcagacacatcctccagcacagcgtacatggacctccgcagcctgacatctgaggattctgcggtctatttctgtgcaagatccccttacgctttctacagtagtagctactggtacttcgatgtctggggcacagggaccacggtcaccgtctcctca

SEQ ID NO: 199 - DNA, 321 bp, synthetic construct
  source 1..321: mol_type = other DNA
gacatcaagatgacccagtctccatcctccatgtatgcatcgctgggagagagagtcactatcacttgcaaggcgagtcaggacattaaaagctatttaagctggtaccagcagaaaccatggaaatctcctaagaccctgatctattatgcaacaagcttggcagatggggtcccatcaagattcagtggcagtggatctgggcaagattattctctaaccatcagcagcctggagtctgacgatacagcaacttattactgtctacagcatggtgagagcccattcacgttcggctcggggacaaagttggaaataaaa

SEQ ID NO: 200 - protein, 122 aa, synthetic construct
QVQLQQSDAELVKPGASVKISCKISGYTFTDQTIHWMNQRPEQGLEWIGYIYPGDGSAMYNEKFRGKATLTADKSSSTAYMQLNSLTSEDSAVYFCARPLYSYASYYWFAYWGQGTLVTVSA

SEQ ID NO: 201 - protein, 5 aa, synthetic construct
DQTIH

SEQ ID NO: 202 - protein, 17 aa, synthetic construct
YIYPGDGSAMYNEKFRG

SEQ ID NO: 203 - protein, 13 aa, synthetic construct
PLYSYASYYWFAY

SEQ ID NO: 204 - protein, 107 aa, synthetic construct
DIQMNQSPSSLSASLGDTITITCHASQNINVWLSWYQRKPGNIPKLLIYKSSNLHTGVPSRFSGSGSGTGFTLTISSLQPEDIATYYCQQGQSYPLTFGGGTKLEIK

SEQ ID NO: 205 - protein, 11 aa, synthetic construct
HASQNINVWLS

SEQ ID NO: 206 - protein, 7 aa, synthetic construct
KSSNLHT

SEQ ID NO: 207 - protein, 9 aa, synthetic construct
QQGQSYPLT

SEQ ID NO: 208 - DNA, 366 bp, synthetic construct
  source 1..366: mol_type = other DNA
caggttcagctgcaacagtctgacgctgagttggtgaaacctggagcttcagtgaagatatcctgcaagatttctggctacaccttcactgaccagactattcactggatgaatcagaggcctgaacagggcctggaatggattggatatatttatcctggagatggtagtgctatgtacaatgagaaattcaggggcaaggccacattgactgcagacaaatcctccagcacagcctacatgcagctcaacagcctgacatctgaagactctgcagtctatttctgtgcaagacctctctattcctacgctagttactactggtttgcttactggggccaagggactctggtcactgtctctgca

SEQ ID NO: 209 - DNA, 321 bp, synthetic construct
  source 1..321: mol_type = other DNA
gacatccagatgaaccagtctccatccagtctgtctgcatcccttggagacacaattaccatcacttgccatgccagtcagaacattaatgtttggttaagctggtaccagcggaaaccaggaaatattcctaaactattgatctataagtcttccaacttgcacacaggcgtcccatcaaggtttagtggcagtggatctggaacaggtttcacattaaccatcagcagcctgcagcctgaagacattgccacttactactgtcaacagggtcaaagttatcctctcacgttcggaggggggaccaagctggaaataaaa

SEQ ID NO: 210 - protein, 117 aa, synthetic construct
QAQLQQSGTELARPGSSVKLSCKASGFTFTIYGINWVKQRTGQGLEWIGEIYPRSGNTYYDEKFKGKATLTADKSTSTAYMELRSLTSEDSAVYFCARSREWEGVFWGQGTLVTVSA

SEQ ID NO: 211 - protein, 5 aa, synthetic construct
IYGIN

SEQ ID NO: 212 - protein, 17 aa, synthetic construct
EIYPRSGNTYYDEKFKG

SEQ ID NO: 213 - protein, 8 aa, synthetic construct
SREWEGVF

SEQ ID NO: 214 - protein, 106 aa, synthetic construct
QIVLTQSPAIMSAFPGEKVTITCSATSSVSYMHWFQQKPGTSPKLWIYSTSNLASGVPARFSGSGSGTSHSLTINRMEAEDAATYYCQQRSRYPFTFGSGTKLEIK

SEQ ID NO: 215 - protein, 10 aa, synthetic construct
SATSSVSYMH

SEQ ID NO: 216 - skipped

SEQ ID NO: 217 - protein, 9 aa, synthetic construct
QQRSRYPFT

SEQ ID NO: 218 - DNA, 351 bp, synthetic construct
  source 1..351: mol_type = other DNA
caggctcagctgcagcagtctggaactgagctggcgaggcctgggtcttcagtgaaactgtcctgcaaggcttctggcttcaccttcacaatttatggtataaactgggtgaagcagagaactggacagggccttgagtggattggagagatttatcctagaagtggtaatacttactacgatgagaagttcaagggcaaggccacactgactgcagacaaatccaccagcacagcgtacatggagctccgcagcctgacatctgaggactctgcggtctatttctgtgcaagatcgagggaatgggaaggggtgttctggggccaggggactctggtcactgtctctgca

SEQ ID NO: 219 - DNA, 318 bp, synthetic construct
  source 1..318: mol_type = other DNA
caaattgttctcacccagtctccagcaatcatgtctgcatttccaggggagaaggtcaccataacctgcagtgccacctcaagtgtaagttacatgcactggttccagcagaagccaggcacttctcccaaactctggatttatagcacatccaacctggcttctggagtccctgctcgcttcagtggcagtggatctgggacctctcactctctcacaatcaaccgaatggaggctgaagatgctgccacttattactgccagcaaaggagtcgttacccattcacgttcggctcggggacaaagttggaaataaaa

SEQ ID NO: 220 - protein, 124 aa, synthetic construct
QVQLQQSEPELARPGASVKLSCTASGYTFINYGVSWVRQRTGQGLEWIGRIYPRSGNTRFNEKFQGKATLTADTSSSTAYMELRSLTSEDSAVYFCTRFPYAFSGNSYWYFDVWGTGTTVTVSS

SEQ ID NO: 221 - protein, 5 aa, synthetic construct
NYGVS

SEQ ID NO: 222 - protein, 17 aa, synthetic construct
RIYPRSGNTRFNEKFQG

SEQ ID NO: 223 - protein, 15 aa, synthetic construct
FPYAFSGNSYWYFDV

SEQ ID NO: 224 - protein, 107 aa, synthetic construct
DIVMTQSHKFMSTSIGDRVSITCKASQDVSAAVAWYQQKPGQSPKLLIYSSSFRYTGVPDRFTGSASGTDFTFTITTVQAEDLAVYYCQQHFDTPYTFGGGTKLEIK

SEQ ID NO: 225 - protein, 11 aa, synthetic construct
KASQDVSAAVA

SEQ ID NO: 226 - protein, 7 aa, synthetic construct
SSSFRYT

SEQ ID NO: 227 - protein, 9 aa, synthetic construct
QQHFDTPYT

SEQ ID NO: 228 - DNA, 372 bp, synthetic construct
  source 1..372: mol_type = other DNA
caggttcagctgcagcagtctgaacctgagctggcgaggcctggggcttcagtgaaactgtcctgcacggcttctggctacaccttcataaactatggtgtaagctgggtgagacagagaactggacagggccttgagtggattggaagaatttatcctagaagtggtaatactcgcttcaatgagaagttccagggcaaggccacactgactgcagacacatcctccagcacagcgtacatggagctccgcagcctgacatctgaggactctgcggtctatttctgtacaagattcccttacgctttctccggtaatagctactggtacttcgatgtctggggcaccgggaccacggtcaccgtctcctca

SEQ ID NO: 229 - DNA, 321 bp, synthetic construct
  source 1..321: mol_type = other DNA
gacattgtgatgacccagtctcacaaattcatgtccacatcaataggagacagggtcagcatcacctgcaaggccagtcaggatgtgagtgctgctgtagcctggtatcaacagaaaccaggtcaatctcctaaactactgatttactcgtcatccttccggtacactggagtccctgatcgcttcactggcagtgcatctgggacggacttcactttcaccatcaccactgtgcaggctgaagacctggcagtttattactgtcagcaacattttgatactccgtacacgttcggaggggggaccaagctggaaataaaa

SEQ ID NO: 230 - protein, 122 aa, synthetic construct
EVKLVESGGGLVQPGGSLKLSCAASGFTFSDYYMYWVRQNAEKRLEWVAYISSGGDAPFYSDNLKGRFTISRDNAKNTLYLQMSRLQPEDTAMYFCAREGFYYYGLYGMDYWGQGTSVTVSS

SEQ ID NO: 231 - protein, 5 aa, synthetic construct
DYYMY

SEQ ID NO: 232 - protein, 17 aa, synthetic construct
YISSGGDAPFYSDNLKG

SEQ ID NO: 233 - protein, 13 aa, synthetic construct
EGFYYYGLYGMDY

SEQ ID NO: 234 - protein, 107 aa, synthetic construct
DIVLSQSPATLSVTPGDSVSLSCRASQSIGDNLHWYQQKSHESPRLIIKSASQSISGIPSRFSGSGSGTDFTLSINIVETEDFGMYFCQQSDTWPLTFGPGTKLELK

SEQ ID NO: 235 - protein, 11 aa, synthetic construct
RASQSIGDNLH

SEQ ID NO: 236 - protein, 7 aa, synthetic construct
SASQSIS

SEQ ID NO: 237 - protein, 9 aa, synthetic construct
QQSDTWPLT

SEQ ID NO: 238 - DNA, 366 bp, synthetic construct
  source 1..366: mol_type = other DNA
gaagtgaagctggtggagtctgggggaggcttagtgcagcctggagggtccctgaaactctcctgtgcagcctctggattcactttcagtgactattacatgtattgggttcgccagaatgcagagaagaggctggagtgggtcgcatacatcagtagtggcggtgatgcccccttttattcagacaatttgaagggccgattcaccatctccagagacaatgccaagaacaccctgtacctgcaaatgagccgtctgcagcctgaggacacagccatgtatttctgtgcaagagaggggttttattactacggtctttatggtatggattattggggtcaaggaacctcagtcaccgtctcctca

SEQ ID NO: 239 - DNA, 321 bp, synthetic construct
  source 1..321: mol_type = other DNA
gatattgtgctatctcagtctccagccaccctatctgtgactccaggagatagcgtcagtctttcctgcagggccagtcagagtattggcgacaacttacactggtatcaacaaaagtcacatgagtctccaaggcttatcatcaagtctgcttcccagtccatctctgggatcccctccaggttcagtggcagtggatcagggacagatttcactctcagtatcaacattgtggagactgaagattttggaatgtatttctgtcaacagagtgacacctggcctctcacgttcggtcctgggaccaagctggagctgaaa

SEQ ID NO: 240 - protein, 118 aa, synthetic construct
EVQLQQSGAELVRPGSSVKMSCKTSGYTFTNYGITWVKQRPGQGLEWIGYIYVGNGYTEYNEKFKDKAALTSDTSSSTAYMQLTSLTSDDSTIYFCARIAQGRCFDYWGQGTTLSVSS

SEQ ID NO: 241 - protein, 5 aa, synthetic construct
NYGIT

SEQ ID NO: 242 - skipped

SEQ ID NO: 243 - protein, 9 aa, synthetic construct
IAQGRCFDY

SEQ ID NO: 244 - protein, 111 aa, synthetic construct
DIVLTQSPASSAVSLGQRATISCRASQSVSTSSYSYMHWYQQKPGQPPKLLIKYASTLESGVPARFSGSGSGTDFTLNIHPVEEEDTATYYCQHSWELPYMFGGGTKLEIK

SEQ ID NO: 245 - protein, 15 aa, synthetic construct
RASQSVSTSSYSYMH

SEQ ID NO: 246 - protein, 7 aa, synthetic construct
YASTLES

SEQ ID NO: 247 - protein, 9 aa, synthetic construct
QHSWELPYM

SEQ ID NO: 248 - DNA, 354 bp, synthetic construct
  source 1..354: mol_type = other DNA
gaggtccagcttcagcagtctggagctgaactggtgaggcctgggtcctcagtgaagatgtcctgcaagacttctggatatacattcacaaattacggtataacctgggtgaaacagaggcctggacagggcctggaatggattggatatatttatgttggaaacggttatactgagtataatgagaagttcaaggacaaggccgcgctgacttcagacacatcctccagcacagcctacatgcagctcaccagcctgacatctgacgactctacaatctatttctgtgccagaatagctcagggaagatgctttgactactggggccaaggcaccactctctcagtctcctca

SEQ ID NO: 249 - DNA, 333 bp, synthetic construct
  source 1..333: mol_type = other DNA
gacattgtactgacacagtctcctgcttcctcagctgtctctctgggacagagggccaccatctcatgcagggccagccaaagtgtcagtacatctagttatagttatatgcactggtaccaacagaaacccggacagccacccaaactcctcattaaatatgcatccacccttgaatctggggtccctgccaggttcagtggcagtggctctgggacagacttcaccctcaacatccatcctgtggaggaggaggatactgcaacatattactgtcagcacagttgggaacttccgtacatgttcggaggggggaccaagctggaaataaaa

SEQ ID NO: 250 - protein, 119 aa, synthetic construct
QVQLQQSGAELARPGTSVKLSCKASGYTFISFGLSWVKQRTGQGLEWIGEIYPRIDNPYYNEKFKGKAILTADKSSSTAYMELRSLTSEDSAVYFCARYFTKGNYFDYWGQGTTLTVSS

SEQ ID NO: 251 - protein, 5 aa, synthetic construct
SFGLS

SEQ ID NO: 252 - protein, 17 aa, synthetic construct
EIYPRIDNPYYNEKFKG

SEQ ID NO: 253 - protein, 10 aa, synthetic construct
YFTKGNYFDY

SEQ ID NO: 254 - protein, 107 aa, synthetic construct
DIQMTQSPASLAASVGETVTITCRASENIYYSLAWYQQKQGKSPQVLIYYANSLEDGVPSRFSGSGSGTQYSLKINSMQPEDTATYFCKQAYDVPFTFGTGTKLELK

SEQ ID NO: 255 - protein, 11 aa, synthetic construct
RASENIYYSLA

SEQ ID NO: 256 - protein, 7 aa, synthetic construct
YANSLED

SEQ ID NO: 257 - protein, 9 aa, synthetic construct
KQAYDVPFT

SEQ ID NO: 258 - DNA, 357 bp, synthetic construct
  source 1..357: mol_type = other DNA
caggttcagctgcagcaatctggagctgagctggcgcggcctgggacttcagtgaagctgtcctgcaaggcttctggctacactttcataagctttggtttaagctgggtgaaacagagaactggacagggccttgagtggattggagagatttatcctagaattgataatccttactacaatgagaagttcaagggcaaggccatactgactgcagacaaatcctccagcacagcgtacatggagctccgcagcctgacatctgaggactctgcggtctatttctgtgcaagatactttactaagggaaactactttgactactggggccaaggcaccactctcacagtctcctca

SEQ ID NO: 259 - DNA, 321 bp, synthetic construct
  source 1..321: mol_type = other DNA
gacatccagatgactcagtctccagcctccctggctgcatctgtgggagaaactgtcaccatcacatgtcgagcaagtgagaacatttactacagtttagcatggtatcagcagaagcaagggaaatctcctcaggtcctgatctattatgcaaacagcttggaagatggtgtcccatcgaggttcagtggcagtggatctgggacacagtattctttgaagatcaatagcatgcagcctgaagataccgcaacttatttctgtaaacaggcttatgacgttccgttcacgttcggtactgggaccaagctggagctgaaa

SEQ ID NO: 260 - protein, 122 aa, synthetic construct
EVQLQESGPELVKPGSSVKISCKASGYTFADHYMNWVKQSHGKSLEWIGDINPNNGGSVYNQKFKGKATLTVDKSSSTAYMELRSLTSEDSAVYFCARRRAHYYDGPYFDYWGQGTTLTVSS

SEQ ID NO: 261 - protein, 5 aa, synthetic construct
DHYMN

SEQ ID NO: 262 - protein, 17 aa, synthetic construct
DINPNNGGSVYNQKFKG

SEQ ID NO: 263 - protein, 13 aa, synthetic construct
RRAHYYDGPYFDY

SEQ ID NO: 264 - protein, 106 aa, synthetic construct
DIVMTQSQKFMSTSVGDRVSLTCKASQIVGTAVAWYQQKPGQSPKLLIYSASIRYTGVPDRFTGSGSGTDFTLTISNMQSEDLADYFCQQYSNYPTFGAGTKLELK

SEQ ID NO: 265 - protein, 11 aa, synthetic construct
KASQIVGTAVA

SEQ ID NO: 266 - protein, 7 aa, synthetic construct
SASIRYT

SEQ ID NO: 267 - protein, 8 aa, synthetic construct
QQYSNYPT

SEQ ID NO: 268 - DNA, 366 bp, synthetic construct
  source 1..366: mol_type = other DNA
gaagtccagttgcaagaatctggacctgaactggtgaagcctgggtcttcagtgaagatatcctgtaaggcttctggatacacgttcgctgaccactacatgaattgggtgaagcagagccatggaaagagccttgagtggattggagatattaatcctaacaatggtggttctgtctacaaccagaagttcaagggcaaggccacattgactgtagacaagtcctccagcacagcctacatggaactccgcagcctgacatctgaggactctgcagtctatttctgtgcaagaaggagggcacattattacgacggtccttactttgactactggggccaaggcaccactctcacagtctcctca

SEQ ID NO: 269 - DNA, 318 bp, synthetic construct
  source 1..318: mol_type = other DNA
gacattgtgatgacccagtctcaaaaattcatgtccacatcagtaggggacagggtcagcctcacctgcaaggccagtcagattgtgggtactgctgtagcctggtatcaacagaaaccaggacaatctcctaaactactgatttactcggcgtccattcggtacactggagtccctgatcgcttcacaggcagtggatctgggacagatttcactctcaccatcagcaatatgcagtctgaagacctggcagattatttctgccagcaatatagcaactatcccacgttcggtgctgggaccaagctggaactgaaa

SEQ ID NO: 270 - protein, 122 aa, synthetic construct
QVQLQQSDAELVKPGASVKISCKVSGYTFTDHTIHWMRQRPEQGLEWIGYIYPGDASSYYNEKFKGKATLTADQSSNTAYIQVNSLTSEDSAVYFCARPLYSYASYYWFAYWGQGTLVTVSA

SEQ ID NO: 271 - protein, 5 aa, synthetic construct
DHTIH

SEQ ID NO: 272 - protein, 17 aa, synthetic construct
YIYPGDASSYYNEKFKG

SEQ ID NO: 273 - skipped

SEQ ID NO: 274 - protein, 107 aa, synthetic construct
DIQMNQSPSSLSASLGDTITITCHASQNINVWLTWYQQKPGNIPKLLIYKASNLHTDVPSRFSGSGSGTGFTLTISSLQPEDIATYYCQQGQSYPLTFGGGTKLEIK

SEQ ID NO: 275 - protein, 11 aa, synthetic construct
HASQNINVWLT

SEQ ID NO: 276 - protein, 7 aa, synthetic construct
KASNLHT

SEQ ID NO: 277 - skipped

SEQ ID NO: 278 - DNA, 366 bp, synthetic construct
  source 1..366: mol_type = other DNA
caggttcagctgcaacagtctgacgctgagttggtgaaacctggagcttcagtgaagatatcctgcaaggtttctggctacaccttcactgaccatactattcactggatgaggcagaggcctgaacagggcctggaatggattggatatatttatcctggagatgctagtagttactacaatgagaagttcaagggcaaggccacattgactgcagaccaatcctccaacacagcctacatacaggtcaacagcctgacatctgaggactctgcagtctatttctgtgcaagacctctctattcctacgctagttactactggtttgcttactggggccaagggactctggtcactgtctctgca

SEQ ID NO: 279 - DNA, 321 bp, synthetic construct
  source 1..321: mol_type = other DNA
gacatccagatgaaccagtctccatccagtctgtctgcatcccttggagacacaattaccatcacttgccatgccagtcagaacattaatgtttggttgacctggtaccagcagaaaccaggaaatattcctaaacttttgatctataaggcttccaacttgcacacagacgtcccatcaaggtttagtggcagtggatctggaacaggtttcacattaaccatcagcagcctgcagcctgaagacattgccacttactactgtcaacagggtcaaagttatcctctcacgttcggaggggggaccaagctggaaataaaa

SEQ ID NO: 280 - protein, 118 aa, synthetic construct
EVQLQQSGAELVRPGSSVKMSCRTSGYTFTNNGITWVKQRPGQGLEWIGYIYVGNGYTEYNEKFKDKATLTSDTASSTVYMQLNSLTSEDSAIYFCARLDRGRALDYWAQGTTLTVSS

SEQ ID NO: 281 - protein, 5 aa, synthetic construct
NNGIT

SEQ ID NO: 282 - skipped

SEQ ID NO: 283 - protein, 9 aa, synthetic construct
LDRGRALDY

SEQ ID NO: 284 - protein, 111 aa, synthetic construct
DIVLTQSPASLAVSLGQRATISCRASKSVSSSAYSYMHWYQQKPGQPPKLLIHLASNLESGVPARFSGSGSGTDFTLNIHPVEEEDAATYYCQHSWELPYTFGGGTKLEIK

SEQ ID NO: 285 - protein, 15 aa, synthetic construct
RASKSVSSSAYSYMH

SEQ ID NO: 286 - protein, 7 aa, synthetic construct
LASNLES

SEQ ID NO: 287 - protein, 9 aa, synthetic construct
QHSWELPYT

SEQ ID NO: 288 - DNA, 354 bp, synthetic construct
  source 1..354: mol_type = other DNA
gaggtccagcttcagcagtctggagctgagctggtgaggcctgggtcctcagtgaagatgtcctgcaggacttctggatacacattcacaaacaacggtataacctgggtgaagcagaggcctggacagggcctggaatggattggatatatttatgttggaaatggttatactgaatacaatgagaagttcaaggacaaggccactctgacttcagacacagcctccagtacagtttacatgcagctcaacagcctgacatctgaggactctgcaatctatttttgtgcaagattggataggggtagggcccttgactactgggcccaaggcaccactctcacagtctcctca

SEQ ID NO: 289 - DNA, 333 bp, synthetic construct
  source 1..333: mol_type = other DNA
gacattgtgctgacacagtctcctgcttccttagctgtatctctggggcagagggccaccatctcatgcagggccagcaaaagtgtcagttcatctgcctatagttatatgcactggtaccaacagaaaccaggacagccacccaaactcctcatccatcttgcatccaacctagaatctggggtccctgccaggttcagtggcagtgggtctgggacagacttcaccctcaatatccatcctgtggaggaggaagatgctgcaacctattactgtcagcacagttgggagcttccttacacgttcggaggggggaccaagctggaaataaaa

SEQ ID NO: 290 - protein, 123 aa, synthetic construct
EVQLQQSVAELVRPGASVKLSCTASGFNIKNTYMHWVKQRPEQGLEWIGRIDPANGNTKYVPKFQGKATITADTSSKTAYLQLSSLTSEDTAIYYCTRWGFDKDGVVYAMDYWGQGTSVTVSS

SEQ ID NO: 291 - protein, 5 aa, synthetic construct
NTYMH

SEQ ID NO: 292 - protein, 17 aa, synthetic construct
RIDPANGNTKYVPKFQG

SEQ ID NO: 293 - protein, 14 aa, synthetic construct
WGFDKDGVVYAMDY

SEQ ID NO: 294 - protein, 107 aa, synthetic construct
DVVLTQSPATLSVTPGDSVSLSCRASQSIYNNLHWYQQKSHESPRLLVKYASQSISGIPSRFSGSGSGTDFTLSINSVETEDFGMYFCQQSNSWPLTFGAGTKLELK

SEQ ID NO: 295 - protein, 11 aa, synthetic construct
RASQSIYNNLH

SEQ ID NO: 296 - skipped

SEQ ID NO: 297 - protein, 9 aa, synthetic construct
QQSNSWPLT

SEQ ID NO: 298 - DNA, 369 bp, synthetic construct
  source 1..369: mol_type = other DNA
gaggttcagctgcagcagtctgtggcagagcttgtgaggccaggggcctcagtcaagttgtcctgcacagcttctggcttcaacattaaaaacacctatatgcactgggtgaaacagaggcctgaacagggcctggagtggattggaaggattgatcctgcgaatggtaatactaaatatgtcccgaagttccagggcaaggccactataactgcagacacatcttctaagacagcctacctgcagctcagcagcctgacatctgaggacactgccatctattactgtactagatggggttttgataaagacggggtggtctatgccatggactattggggtcaaggaacctcagtcaccgtctcctca

SEQ ID NO: 299 - DNA, 321 bp, synthetic construct
  source 1..321: mol_type = other DNA
gatgttgtactaacccagtctccagccaccctgtctgtgactccaggagatagcgtcagtctttcctgcagggccagccaaagtatttacaacaacctacactggtatcaacaaaaatcacatgagtctccaaggcttctcgtcaagtatgcttcccagtccatctctgggatcccctccaggttcagtggcagtggatcggggacagatttcactctcagtatcaacagtgtggagacagaagattttggaatgtatttctgtcaacagagtaacagctggcctctcacgttcggtgctgggaccaagctggagctgaaa

SEQ ID NO: 300 - protein, 119 aa, synthetic construct
DVKLVESGEGLVKPGGSLKLSCAVSGFTFSNYAMSWVRQTPEKRLEWVAYISSGGDFIYYRDTVKGRFTISRDNARNTLYLQMSSLKSEDTAMYYCILQLRPEAWFAYWGQGTLVTVSA

SEQ ID NO: 301 - skipped

SEQ ID NO: 302 - protein, 17 aa, synthetic construct
YISSGGDFIYYRDTVKG

SEQ ID NO: 303 - protein, 10 aa, synthetic construct
QLRPEAWFAY

SEQ ID NO: 304 - protein, 107 aa, synthetic construct
DIKMTQSPSSMYASLGERVTITCKASQDIKNYLSWFQQIPGKSPKTLIYRANRLVDGVPSRFSGSGSGQDYSLTISSLEYEDMGIYYCLQYYEFPYTFGGGTKLEIK

SEQ ID NO: 305 - protein, 11 aa, synthetic construct
KASQDIKNYLS

SEQ ID NO: 306 - skipped

SEQ ID NO: 307 - protein, 9 aa, synthetic construct
LQYYEFPYT

SEQ ID NO: 308 - DNA, 357 bp, synthetic construct
  source 1..357: mol_type = other DNA
gacgtgaagctggtggaatctggggaaggcttagtgaagcctggagggtccctgaaactctcctgtgcagtctctggattcactttcagtaactatgccatgtcttgggttcgccagactccagagaagaggctggagtgggtcgcatatattagtagtggtggtgatttcatctactatagagacactgtgaagggccgattcaccatctccagagacaatgccaggaacaccctgtacctgcaaatgagcagtctgaagtctgaggacacagccatgtattattgtattttacaactacggccagaagcctggtttgcttactggggccaagggactctggtcactgtctctgca

SEQ ID NO: 309 - DNA, 321 bp, synthetic construct
  source 1..321: mol_type = other DNA
gacatcaagatgacccagtctccatcttccatgtatgcatctctaggagagagagtcactatcacttgcaaggcgagtcaggacattaaaaactatttaagctggttccaacagataccagggaaatctcctaagaccctgatctatcgtgcaaacagattggtagatggggtcccatcaaggttcagtggcagtggatctgggcaagattattctctcaccatcagcagcctggagtatgaagatatgggaatttattattgtctacagtattatgagtttccgtacacgttcggaggggggaccaaactggaaataaaa

SEQ ID NO: 310 - protein, 112 aa, synthetic construct
QVQLQQSGAELARPGASVKLSCKASGHTFTSYGISWVKQRTGQGLEWIGKIYPRSGNTYYDEKFKGKATLTADKPSTTAYMELRSLTSEDSAVYFCATRDYWGQGTTLTVSS

SEQ ID NO: 311 - skipped

SEQ ID NO: 312 - protein, 17 aa, synthetic construct
KIYPRSGNTYYDEKFKG

SEQ ID NO: 313 - skipped

SEQ ID NO: 314 - protein, 106 aa, synthetic construct
QIVLTQSPVIMSASPGEKVTITCSASSSVSYMYWFQQKPGTSPKFWIYSTSNLASGVPARFSGSGSGTSYSLTISRMEAEDAATYYCQQRSRYPWTFGGGTKLEIK

SEQ ID NO: 315 - protein, 10 aa, synthetic construct
SASSSVSYMY

SEQ ID NO: 316 - skipped

SEQ ID NO: 317 - protein, 9 aa, synthetic construct
QQRSRYPWT

SEQ ID NO: 318 - DNA, 336 bp, synthetic construct
  source 1..336: mol_type = other DNA
caggttcagctgcagcagtctggagctgagctggcgaggcctggggcttcagtgaagctgtcctgcaaggcttctggccacaccttcacaagctatggtataagttgggtgaagcagagaactggacagggccttgagtggattggaaagatttatcctagaagcggtaatacttactacgatgagaagttcaagggcaaggccacactgactgcagacaaaccctccaccacagcgtacatggagctccgcagcctgacatctgaggactctgcggtctatttctgtgcaaccagggactactggggccaaggcaccactctcacagtctcctca

SEQ ID NO: 319 - DNA, 318 bp, synthetic construct
  source 1..318: mol_type = other DNA
caaattgttctcacccagtctccagtaatcatgtctgcatctccaggggagaaggtcaccataacctgcagtgccagctcaagtgtaagttacatgtactggttccagcagaagccaggcacttctcccaaattctggatttatagcacatccaacctggcttctggagtccctgctcgcttcagtggcagtggatctgggacctcttactctctcacaatcagccgaatggaggctgaagatgctgccacttattactgccagcaaaggagtaggtacccgtggacgttcggtggaggcaccaagctggaaatcaag

SEQ ID NO: 320 - protein, 117 aa, synthetic construct
QVQLQQSGAELARPGASVKLSCKASGFSFTSYGISWVKQRTGQGLEWIGQIYLRSSNTYYNEKFKDKATLTADKSSSTVYMELRSLTSEDSAVYFCARYYGSSSDYWGQGTTLTVSS

SEQ ID NO: 321 - skipped

SEQ ID NO: 322 - protein, 17 aa, synthetic construct
QIYLRSSNTYYNEKFKD

SEQ ID NO: 323 - protein, 8 aa, synthetic construct
YYGSSSDY

SEQ ID NO: 324 - protein, 107 aa, synthetic construct
DIQMNQSPSSLSASLGDTITINCHASQNINVWLSWYQQKPGNIPKVLIYQASNLQTGVPTRFSGSGSGTGFTLTISRLQPEDIATYYCQQGQRYPWTFGGGTKLEIK

SEQ ID NO: 325 - skipped

SEQ ID NO: 326 - protein, 7 aa, synthetic construct
QASNLQT

SEQ ID NO: 327 - protein, 9 aa, synthetic construct
QQGQRYPWT

SEQ ID NO: 328 - DNA, 351 bp, synthetic construct
  source 1..351: mol_type = other DNA
caggttcagctgcagcagtctggagctgaactggcgaggcctggggcttcagtgaagctgtcctgcaaggcttctggcttcagtttcacaagttatggtataagttgggtgaagcagagaactggacagggccttgagtggattggacagatttatcttagaagtagtaatacttactacaatgagaagttcaaggacaaggccacactgactgcagacaaatcctccagcacagtgtacatggagctccgcagcctgacatctgaggactctgcggtctatttctgtgcaagatactacggtagtagttcggactactggggccaaggcaccactctcacagtgtcctca

SEQ ID NO: 329 - DNA, 321 bp, synthetic construct
  source 1..321: mol_type = other DNA
gacatccagatgaaccagtctccatccagtctgtctgcatcccttggagacacaatcaccatcaattgtcatgccagtcagaacattaatgtttggttaagctggtaccagcagaaaccaggaaatattcctaaagttttgatctatcaggcttccaacttgcagacaggcgtcccaacaagatttagtggcagtggatctggaacaggtttcactttaaccatcagcagactgcagcctgaagacattgccacttactactgtcagcagggtcaaagatatccgtggacgttcggtggaggcaccaagctggaaatcaag

SEQ ID NO: 330 - protein, 122 aa, synthetic construct
QVQLQQSDAELVKPGASVKISCKVSGYALTDHSIHWMKQRPEQGLAWIGYMYPGDGSANYNDKFRGKATLTADISSSTAYMHLNSLTSEDSAVYFCTRPYSFGSSYDYFGYWGQGTTLTVSS

SEQ ID NO: 331 - protein, 5 aa, synthetic construct
DHSIH

SEQ ID NO: 332 - protein, 17 aa, synthetic construct
YMYPGDGSANYNDKFRG

SEQ ID NO: 333 - protein, 13 aa, synthetic construct
PYSFGSSYDYFGY

SEQ ID NO: 334 - protein, 107 aa, synthetic construct
DIQMNQSPSSLSASLGDTITITCHASQNINVWLNWYQQKPGNIPKLLIYKASKLHTGVSSSFSGSGSGTGFTLTISSLQPEDIGTYYCQQGQSYPYTFGGGTKLEIK

SEQ ID NO: 335 - protein, 11 aa, synthetic construct
HASQNINVWLN

SEQ ID NO: 336 - protein, 7 aa, synthetic construct
KASKLHT

SEQ ID NO: 337 - protein, 9 aa, synthetic construct
QQGQSYPYT

SEQ ID NO: 338 - DNA, 366 bp, synthetic construct
  source 1..366: mol_type = other DNA
caggttcagctgcaacagtctgacgctgagttggtgaaacctggagcttcagtgaagatatcctgcaaggtttctggctacgccctcactgaccattctattcattggatgaagcagaggcctgaacagggcctggcatggattggatatatgtatcctggagatggtagtgctaactacaatgacaaattcaggggcaaggccacattgactgcagacatatcctccagcacagcctacatgcacctcaacagcctgacatctgaggactctgcagtctatttctgtacaagaccctattccttcggcagtagttacgactactttggctactggggccaaggcaccactctcacagtctcctca

SEQ ID NO: 339 - DNA, 321 bp, synthetic construct
  source 1..321: mol_type = other DNA
gacatccagatgaaccagtctccatccagtctgtctgcctcccttggagacacaattaccatcacttgccatgccagtcagaacattaatgtttggttaaactggtaccagcagaaaccaggaaatattcctaaactattgatctataaggcttccaagttgcacacaggcgtctcatcaagctttagtggcagtggatctggaacaggtttcacattaaccatcagcagcctacagcctgaagacattggcacttactactgtcaacagggtcaaagttatccgtacacgttcggaggggggaccaagctggagataaaa

SEQ ID NO: 340 - protein, 117 aa, synthetic construct
QVQLQQSGVELARPGASVKLSCKASGFSFTSYGISWVKQKTGQGLEWIGQIYLRSSNTYYSEKFKDRATLTADKSSSTVYMELRSLTSEDSAVYFCARYYGSSSDYWGQGTTLTVSS

SEQ ID NO: 341 - skipped

SEQ ID NO: 342 - protein, 17 aa, synthetic construct
QIYLRSSNTYYSEKFKD

SEQ ID NO: 343 - skipped

SEQ ID NO: 344 - protein, 107 aa, synthetic construct
DIQMNQSPSSLSASLGDTITINCHASQNINVWLSWYQQKPGNIPKVLINQASNLQTDVPTRFSGSGSGTDFKLIISRLQSEDIATYYCQQGQSYPWTFGGGTKLEIK

SEQ ID NO: 345-346 - skipped (2 entries)

SEQ ID NO: 347 - protein, 9 aa, synthetic construct
QQGQSYPWT

SEQ ID NO: 348 - DNA, 351 bp, synthetic construct
  source 1..351: mol_type = other DNA
caggttcagctgcagcagtcgggagttgaactggcgaggcctggggcttcagtgaagttgtcctgcaaggcttcgggcttcagttttacaagttatggtataagctgggtgaagcagaaaactggacagggccttgagtggattggacagatttatcttagaagtagtaatacttattacagtgagaagttcaaggacagggccacactgactgcagacaaatcctccagcacagtgtacatggagctccgcagcctgacatctgaggactctgcggtctatttctgtgcaagatattacggtagtagttcggactactggggccaaggcaccactctcacagtctcctca

SEQ ID NO: 349 - DNA, 321 bp, synthetic construct
  source 1..321: mol_type = other DNA
gacatccagatgaaccagtctccatccagtctgtctgcatcccttggagacacaatcaccatcaattgccatgccagtcagaacattaatgtttggctaagctggtaccagcagaaaccaggaaatattcctaaagttttgatcaatcaggcttccaacttgcagacagacgtcccaacaagatttagtggcagtggatctggaacagatttcaaattaatcatcagcagactgcagtctgaggacattgccacttactactgtcagcagggtcaaagttatccgtggacgttcggtggaggcaccaagctggaaatcaaa

SEQ ID NO: 350 - protein, 122 aa, synthetic construct
QVQLQQSDAELVKPGASVKISCKVSGYALTDHSIHWMKQRPEQGLAWIGYMYPGDGSANYNDKFKGKATLTADISSSTAYMQLNSLTSEDSAVYFCTRPYYFGSSYDYFGYWGQGTTLTVSS

SEQ ID NO: 351 - skipped

SEQ ID NO: 352 - protein, 17 aa, synthetic construct
YMYPGDGSANYNDKFKG

SEQ ID NO: 353 - protein, 13 aa, synthetic construct
PYYFGSSYDYFGY

SEQ ID NO: 354 - protein, 107 aa, synthetic construct
DIQMNQSPSSLSASLGDTITITCHASQNINVWLSWYQQKPGNVPKLLIYKASKLHTGVSSNFSGSGSGTGFTLTISSLQPEDIGTYYCQQGQSYPYTFGGGTKLEIK

SEQ ID NO: 355-357 - skipped (3 entries)

SEQ ID NO: 358 - DNA, 366 bp, synthetic construct
  source 1..366: mol_type = other DNA
caggttcagctgcaacagtctgacgctgagttggtgaaacctggagcttcagtgaagatatcctgcaaggtttctggctacgccctcactgaccattctattcactggatgaagcagaggcctgaacagggcctggcatggattggatatatgtatcctggagatggtagtgctaattacaatgacaaattcaagggcaaggccacattgactgcagacatatcctccagcacagcctacatgcagctcaacagcctgacatctgaggattctgcagtctatttctgtacaagaccctattacttcggcagtagttacgactactttggctactggggccaaggcaccactctcacagtctcctca

SEQ ID NO: 359 - DNA, 321 bp, synthetic construct
  source 1..321: mol_type = other DNA
gacatccagatgaaccagtctccatccagtctgtctgcctcccttggagacacaattaccatcacttgccatgccagtcagaacattaatgtttggttaagctggtaccagcagaaaccaggaaatgttcctaaattattgatctataaggcttccaagttgcacacaggcgtctcatcaaactttagtggcagtggatctggaacaggtttcacattaaccatcagcagcctgcagcctgaagacattggcacttactactgtcaacagggtcaaagttatccgtacacgttcggaggggggaccaagctggagataaaa

SEQ ID NO: 360 - protein, 112 aa, synthetic construct
QVQLQQSGVELARPGASVKLSCKASGSTFTSYSITWVKQRTGQGLEWIGVIYPRSDNTYYDEKFKGKATLTADKSSSTAYIELRSLTSEDSAVYFCATRDYWGQGTTLTVSS

SEQ ID NO: 361 - protein, 5 aa, synthetic construct
SYSIT

SEQ ID NO: 362 - protein, 17 aa, synthetic construct
VIYPRSDNTYYDEKFKG

SEQ ID NO: 363 - skipped

SEQ ID NO: 364 - protein, 106 aa, synthetic construct
QIVLTQSPAIMSASPGEKVTITCSASSSVSYMYWFQQKPGTSPKFWIYSTSNLASGVPARFSGSGSGTSYSLTISRMEAEDAATYYCQQRSNYPFTFGSGTKLEIK

SEQ ID NO: 365-366 - skipped (2 entries)

SEQ ID NO: 367 - protein, 9 aa, synthetic construct
QQRSNYPFT

SEQ ID NO: 368 - DNA, 336 bp, synthetic construct
  source 1..336: mol_type = other DNA
caggttcagctgcagcagtctggagttgagctggcgaggcctggggcttcagtgaagctgtcctgcaaggcttctggctccaccttcacaagctatagtataacctgggtgaagcagagaactggacagggccttgagtggatcggagtgatttatcctagaagtgataatacttactacgatgagaagttcaagggcaaggccacactgactgcagacaaatcctccagcacagcgtacatagagctccgcagcctgacatctgaggactctgcggtctatttctgtgcgacacgggactactggggccaaggcaccactctcacagtctcctca

SEQ ID NO: 369 - DNA, 318 bp, synthetic construct
  source 1..318: mol_type = other DNA
caaattgttctcacccagtctccagcaatcatgtctgcatctccaggggagaaggtcaccataacctgcagtgccagctcaagtgtgagttacatgtactggttccagcagaagccaggcacttctcccaaattctggatttatagcacatccaacctggcttctggagtccctgctcgcttcagtggcagtggatctgggacctcttactctctcacaatcagccgaatggaggctgaagatgctgccacttattactgccagcaaaggagtaattacccattcacgttcggctcggggacaaagttggaaataaaa

SEQ ID NO: 370 - protein, 117 aa, synthetic construct
RVQLQQAGAELVKPGASVKVSCKASGNTFNNYWIYWVKQRPGQGLEWIGRIHPSDSGTNYNQKFEGKATLTVDKSSGTAYMQLSSLTAEDSAVYYCALLSLYAMDSWGHGTSVTVSS

SEQ ID NO: 371 - protein, 5 aa, synthetic construct
NYWIY

SEQ ID NO: 372 - protein, 17 aa, synthetic construct
RIHPSDSGTNYNQKFEG

SEQ ID NO: 373 - protein, 8 aa, synthetic construct
LSLYAMDS

SEQ ID NO: 374 - protein, 111 aa, synthetic construct
DIVLTQSPASLAVSLGQRATISCRASKSVSSFGYNYIHWYLQKPGQPPKLLIYLASDLESGVPARFSGSGSGTGFTLNIHPVEEEDAATYYCQHSREFPWTFGGGTKLEIK

SEQ ID NO: 375 - protein, 15 aa, synthetic construct
RASKSVSSFGYNYIH

SEQ ID NO: 376 - protein, 7 aa, synthetic construct
LASDLES

SEQ ID NO: 377 - protein, 9 aa, synthetic construct
QHSREFPWT

SEQ ID NO: 378 - DNA, 351 bp, synthetic construct
  source 1..351: mol_type = other DNA
cgggtccaactgcagcaggctggggctgaactggtgaagcctggggcttcagtgaaggtttcctgcaaggcctcaggcaacaccttcaacaactactggatatattgggtgaaacagaggcctggccaaggccttgagtggattggaaggattcatccttctgatagtggaactaactacaatcaaaagttcgagggcaaggccacattgactgttgacaaatcctccggcactgcctacatgcagctcagcagcctgacagctgaggactctgcggtctattactgtgcactcctatcgctctatgctatggactcctggggtcacggaacctcagtcaccgtctcctca

SEQ ID NO: 379 - DNA, 333 bp, synthetic construct
  source 1..333: mol_type = other DNA
gacattgtgctgacacagtctcctgcttccttagctgtatctctggggcagagggccaccatctcatgcagggccagcaaaagtgtcagttcttttggctataattatatacactggtacctacagaaaccaggacagccacccaaactcctcatctatcttgcatccgacctagaatctggggtccctgccaggttcagtggcagtgggtctgggacaggcttcaccctcaacatccatcctgtggaggaagaggatgctgcaacctattactgtcagcacagtagggagtttccgtggacgttcggtggaggcaccaagctggaaatcaaa

SEQ ID NO: 380 - protein, 114 aa, synthetic construct
QVQLQQPGTELVKPGASVKLSCRASGWTHWVKQRPGQGLEWIGNINLSNGRTNYNEKFRAMATLTVDKSSSTAYTQLSSLTSEDSAVYYCANSMVPHFFPMDYWGQGTSVTVSS

SEQ ID NO: 381 - skipped

SEQ ID NO: 382 - protein, 17 aa, synthetic construct
NINLSNGRTNYNEKFRA

SEQ ID NO: 383 - protein, 11 aa, synthetic construct
SMVPHFFPMDY

SEQ ID NO: 384 - protein, 107 aa, synthetic construct
NIVMTQYPKSMSLSIGERVTLRCKASENVGSYVSWYQQKPEQSPKLLIYGASNRYTGVPDRFTGSGSATDFILTITSVQAEDLADYHCGQSYNYPLTFGAGTKLELK

SEQ ID NO: 385 - protein, 11 aa, synthetic construct
KASENVGSYVS

SEQ ID NO: 386 - protein, 7 aa, synthetic construct
GASNRYT

SEQ ID NO: 387 - protein, 9 aa, synthetic construct
GQSYNYPLT

SEQ ID NO: 388 - DNA, 342 bp, synthetic construct
  source 1..342: mol_type = other DNA
caggtccaactgcagcagcctgggactgaattggtgaagcctggggcctcagtgaaactgtcctgcagggcttctggctggacgcactgggtgaaacagaggcctggacaaggccttgagtggattggaaatattaatcttagcaatggtcgtaccaactacaatgagaagttcagggccatggccacgctgactgtagacaaatcctccagcacagcctacacgcagctcagcagcctgacttctgaggactctgcggtctattattgtgcaaactcgatggtgccgcatttttttcctatggactattggggtcaaggaacctcagtcaccgtctcctca

SEQ ID NO: 389 - DNA, 321 bp, synthetic construct
  source 1..321: mol_type = other DNA
aacattgtaatgacccaatatcccaaatccatgtccctgtcaataggagagagggtcaccttgaggtgcaaggccagtgaaaatgtgggtagttatgtttcgtggtatcaacagaaaccagagcagtctccaaaattgctgatatacggggcttccaaccgttacactggggtccccgatcgcttcactggcagtggatctgcaacagatttcattctgaccatcaccagtgtgcaggctgaagaccttgcagattatcactgtggacagagttacaactatccgctcacgttcggtgccgggaccaagctggagctgaaa

SEQ ID NO: 390 - protein, 122 aa, synthetic construct
QVQLQQSDAELVKPGGSVKISCKVSGYTFTDHTIHWMKQRPEQGLEWIGNIYPGDGSTMYNEKFKGKATLTADKSSSTAYMHLNSLTSEDSAVYFCARPYYFGSASYYFGYWGQGTTLTVSS

SEQ ID NO: 391 - skipped

SEQ ID NO: 392 - protein, 17 aa, synthetic construct
NIYPGDGSTMYNEKFKG

SEQ ID NO: 393 - protein, 13 aa, synthetic construct
PYYFGSASYYFGY

SEQ ID NO: 394 - protein, 107 aa, synthetic construct
DIQMNQSPSSLSASLGDTITITCHASQNINVWLNWYQQKPGNIPKLLIYKASNLHTGVPSRFSGSGSGIGFTLTISSLQPEDIATYYCQQGQSYPLTFGGGTKLEIK

SEQ ID NO: 395-397 - skipped (3 entries)

SEQ ID NO: 398 - DNA, 366 bp, synthetic construct
  source 1..366: mol_type = other DNA
caggttcagctgcagcagtctgacgctgagttggtgaaacctggaggttcagtgaagatatcctgcaaggtttctggctacaccttcactgaccatactattcactggatgaagcagaggcctgaacagggcctggaatggattggaaatatttatcctggagatggtagtactatgtacaatgagaagttcaagggcaaggccacattgactgcagacaaatcctccagcacagcctacatgcacctcaacagcctgacatctgaggactctgcagtctatttctgtgcaagaccctattacttcggtagtgcctcgtactactttggctactggggccaaggcaccactctcacagtctcctca

SEQ ID NO: 399 - DNA, 321 bp, synthetic construct
  source 1..321: mol_type = other DNA
gacatccagatgaaccagtctccatccagtctgtctgcatcccttggagacacaattaccatcacttgccatgccagtcagaacattaatgtttggttaaactggtaccagcagaaaccaggaaatattcctaaactattgatctataaggcttccaacttgcacacaggcgtcccatcaagatttagtggcagtggctctggaataggtttcacattaaccatcagcagcctgcagcctgaagacattgccacttactactgtcaacagggtcaaagttatcctctgacgttcggtggaggcaccaagctggaaatcaaa

SEQ ID NO: 400 - protein, 122 aa, synthetic construct
EVQLVQSGAEVKKPGESLKISCKISGYTFTDQTIHWMHQMPGKGLEWIGYIYPGDGSAMYNEKFRGQATLSADKSISTAYLQWSSLKASDTAMYFCARPLYSYASYYWFAYWGQGTLVTVSS

SEQ ID NO: 401-403 - skipped (3 entries)

SEQ ID NO: 404 - protein, 107 aa, synthetic construct
DIQMTQSPSTLSASVGDRVTITCHASQNINVWLSWYQRKPGKAPKLLIYKSSNLHTGVPSRFSGSGSGTEFTLTISSLQPDDFATYYCQQGQSYPLTFGQGTKLEIK

SEQ ID NO: 405-407 - skipped (3 entries)

SEQ ID NO: 408 - DNA, 366 bp, synthetic construct
  source 1..366: mol_type = other DNA
gaagtacagctcgtgcagtcgggtgccgaggtcaagaagccgggcgagagcttgaagatctcatgtaagatcagcggctataccttcactgatcagaccatacactggatgcatcagatgcctgggaaaggtctagaatggatcggctatatttacccaggagatggatccgcaatgtataacgagaagtttaggggacaggctacactgagtgcagacaaatccattagcactgcgtacctgcaatggagttctctcaaagccagcgacaccgctatgtacttttgcgccagacccctttactcttatgcctcttactactggttcgcttattggggccaagggacgctggttacagtgtcatcc

SEQ ID NO: 409 - DNA, 321 bp, synthetic construct
  source 1..321: mol_type = other DNA
gatattcagatgacccaatccccctctaccctatccgcttccgtgggggaccgggtgacaatcacgtgccatgcaagccagaatatcaacgtttggctgagctggtaccagagaaagccaggaaaggcccctaaacttttgatttacaagtcatccaacttacacacaggagtcccctcgaggtttagcggctcaggtagtggcacagaattcactctgactatctctagtctgcagccagatgacttcgccacctactattgtcaacaggggcagtcttatcctctcacctttggccagggtactaaactcgagataaag

SEQ ID NO: 410 - protein, 122 aa, synthetic construct
EVQLVQSGAEVKKPGESLKISCKISGYTFTDQTIHWVHQMPGKGLEWMGYIYPGDGSAMYNEKFQGQVTISADKSISTAYLQWSSLKASDTAMYYCARPLYSYASYYWFAYWGQGTLVTVSS

SEQ ID NO: 411 - skipped

SEQ ID NO: 412 - protein, 17 aa, synthetic construct
YIYPGDGSAMYNEKFQG

SEQ ID NO: 413 - skipped

SEQ ID NO: 414 - protein, 107 aa, synthetic construct
EIVLTQSPATLSLSPGERATLSCHASQNINVWLSWYQRKPGQAPKLLIYKSSNLHTGIPARFSGSGSGTDFTLTISSLEPEDFAVYYCQQGQSYPLTFGQGTKLEIK

SEQ ID NO: 415-417 - skipped (3 entries)

SEQ ID NO: 418 - DNA, 366 bp, synthetic construct
  source 1..366: mol_type = other DNA
gaagttcaactggtgcagtctggagccgaggtcaagaaacccggggagagcctaaagatttcctgcaagatatcaggttatactttcacagaccagacaattcattgggtgcaccagatgccgggcaagggactggaatggatgggttacatctatcctggggatggaagcgctatgtacaacgagaaattccagggccaagtaaccatcagtgctgataagtctatctccaccgcatacctccagtggtccagccttaaagcatcagacaccgccatgtattactgtgccaggccactgtattcgtacgctagttactactggtttgcgtattggggccagggcactttggtcacggtgtcttcc

SEQ ID NO: 419 - DNA, 321 bp, synthetic construct
  source 1..321: mol_type = other DNA
gaaattgttctgactcagagcccagctaccttgtccctgtcccctggagagagggcaactctttcgtgccatgcttctcagaatataaacgtctggttgagctggtaccagagaaagcccggacaggccccgaaactgctgatctataagtcttctaacctccacacaggcatccctgcgcggttcagtggttcaggctcagggaccgacttcacgttaacaatcagctccctcgagcccgaggattttgccgtgtactattgtcaacaagggcagagttacccactaacctttggccagggtacaaagctggaaattaaa

SEQ ID NO: 420 - protein, 122 aa, synthetic construct
QVQLVQSGAEVKKPGSSVKVSCKISGYSFTDQTIHWMRQAPGQGLEWIGYIYPGDGSAMYNEKFRGRATLTADKSTSTAYMELSSLRSEDTAVYFCARPLYSYASYYWFAYWGQGTLVTVSS

SEQ ID NO: 421-423 - skipped (3 entries)

SEQ ID NO: 424 - protein, 107 aa, synthetic construct
EIVLTQSPATLSLSPGERATLSCHASQNINVWLSWYQQKPGQAPRLLIYKSSNLHTGIPARFSGSGSGTDFTLTISSLEPEDFAVYYCQQGQSYPLTFGQGTKLEIK

SEQ ID NO: 425-427 - skipped (3 entries)

SEQ ID NO: 428 - DNA, 366 bp, synthetic construct
  source 1..366: mol_type = other DNA
caggtgcaactggtccagagcggggctgaggtgaagaagccagggtctagtgtgaaggtctcctgcaagatctccggttattccttcacagatcagacgattcactggatgaggcaggctcccggtcaaggactggaatggatcggctacatatacccgggcgacggaagtgcaatgtataacgaaaaattccggggccgcgccactttgactgcagacaaatcaaccagcacagcctacatggagctctcttctcttcgatcagaggataccgctgtgtacttttgtgcgagacctctgtattcttacgcctcctattactggtttgcctattggggccagggaaccctagttacagtaagctcg

SEQ ID NO: 429 - DNA, 321 bp, synthetic construct
  source 1..321: mol_type = other DNA
gaaatcgtcctcacgcagtcacctgcaacactgtctttgagcccaggcgagagagccaccctgtcctgccatgcgtctcagaacatcaacgtgtggctgagctggtatcagcaaaaaccggggcaagctccacggctgctaatctacaagtcctcaaatcttcacacaggaattcctgccaggtttagtggctctgggtcgggcaccgacttcactttaaccataagttccctggagcccgaggatttcgctgtttattactgtcagcagggtcagagctaccccttgacatttggtcagggaactaaactcgaaattaag

SEQ ID NO: 430 - protein, 122 aa, synthetic construct
QVQLVQSGAEVKKPGSSVKVSCKISGYSFTDQTIHWVRQAPGQGLEWMGYIYPGDGSAMYNEKFQGRVTITADKSTSTAYMELSSLRSEDTAVYYCARPLYSYASYYWFAYWGQGTLVTVSS

SEQ ID NO: 431-433 - skipped (3 entries)

SEQ ID NO: 434 - protein, 107 aa, synthetic construct
EIVLTQSPATLSLSPGERATLSCHASQNINVYLSWYQQKPGQAPRLLIYKSSNLHTGIPARFSGSGSGTDFTLTISSLEPEDFAVYYCQQGQSYPLTFGQGTKLEIK

SEQ ID NO: 435 - protein, 11 aa, synthetic construct
HASQNINVYLS

SEQ ID NO: 436-437 - skipped (2 entries)

SEQ ID NO: 438 - DNA, 366 bp, synthetic construct
  source 1..366: mol_type = other DNA
caggtccaactcgtacagagtggtgccgaggttaagaagccggggtcttccgttaaagtgtcctgtaagatctctggctactcattcacagatcaaactattcactgggtgcggcaggctcccgggcagggtcttgaatggatgggatatatctacccaggagacggcagcgctatgtataacgagaagttccagggacgcgtgaccataacagcagacaaatccactagtacggcctatatggagctgagctcactaaggagcgaagataccgccgtctactattgcgctagacctctgtactcgtacgcatcttattactggtttgcgtactggggccagggcacattggtgaccgtgtccagc

SEQ ID NO: 439 - DNA, 321 bp, synthetic construct
  source 1..321: mol_type = other DNA
gagatcgtcctgacacagtcccctgccacactgtcactgtctccaggcgagagagctaccctgtcttgtcacgccagccagaacatcaacgtgtacctgtcctggtatcagcagaagcccggccaggctcctagactgctgatctacaagtcctccaacctgcacacaggcatccccgccagattttccggctctggctctggcacagactttaccctgaccatctccagcctggaacctgaggacttcgccgtgtactactgtcagcagggccagtcttaccctctgacctttggccagggcaccaagctggaaatcaag

SEQ ID NO: 440 - protein, 122 aa, synthetic construct
QVQLVQSGAEVKKPGSSVKVSCKISGYTFTDQTIHWVRQAPGQGLEWMGYIYPGDGSAMYNEKFQGRVTITADKSTSTAYMELSSLRSEDTAVYYCARPLYSYASYYWFAYWGQGTLVTVSS

SEQ ID NO: 441-447 - skipped (7 entries)

SEQ ID NO: 448 - DNA, 366 bp, synthetic construct
  source 1..366: mol_type = other DNA
caggtgcagctggttcagtctggcgccgaagtgaagaaacctggctcctccgtgaaggtgtcctgcaagatctccggctacacctttaccgaccagaccatccactgggtccgacaggctccaggacaaggcttggagtggatgggctacatctatcctggcgacggctccgccatgtacaacgagaagtttcagggcagagtgaccatcaccgccgacaagtctacctccaccgcctacatggaactgtccagcctgagatccgaggacaccgccgtgtactactgtgccagacctctgtactcctacgcctcctactattggttcgcctactggggccagggcaccctggttacagtttcttct

SEQ ID NO: 449 - skipped

SEQ ID NO: 450 - protein, 122 aa, synthetic construct
QVQLVQSGAEVKKPGSSVKVSCKASGYTFTDQTIHWVRQAPGQGLEWMGYIYPGDGSAMYNEKFQGRVTITADKSTSTAYMELSSLRSEDTAVYYCARPLYSYASYYWFAYWGQGTLVTVSS

SEQ ID NO: 451-457 - skipped (7 entries)

SEQ ID NO: 458 - DNA, 366 bp, synthetic construct
  source 1..366: mol_type = other DNA
caggtgcagctggttcagtctggcgccgaagtgaagaaacctggctcctccgtgaaggtgtcctgcaaggcttctggctacacctttaccgaccagaccatccactgggtccgacaggctccaggacaaggcttggagtggatgggctacatctatcctggcgacggctccgccatgtacaacgagaagtttcagggcagagtgaccatcaccgccgacaagtctacctccaccgcctacatggaactgtccagcctgagatctgaggacaccgccgtgtactactgcgccagacctctgtactcctacgcctcctactattggttcgcctactggggccagggcaccctggttacagtttcttct

SEQ ID NO: 459 - skipped

SEQ ID NO: 460 - protein, 122 aa, synthetic construct
QVQLVQSGAEVKKPGSSVKVSCKISGYTFTDQTIHWVRQAPGQGLEWMGYIYPGDSSAMYNEKFQGRVTITADKSTSTAYMELSSLRSEDTAVYYCARPLYSYASYYWFAYWGQGTLVTVSS

SEQ ID NO: 461 - skipped

SEQ ID NO: 462 - protein, 17 aa, synthetic construct
YIYPGDSSAMYNEKFQG

SEQ ID NO: 463-467 - skipped (5 entries)

SEQ ID NO: 468 - DNA, 366 bp, synthetic construct
  source 1..366: mol_type = other DNA
caggtgcagctggttcagtctggcgccgaagtgaagaaacctggctcctccgtgaaggtgtcctgcaagatctccggctacacctttaccgaccagaccatccactgggtccgacaggctccaggacaaggcttggagtggatgggctacatctaccctggcgactcctccgccatgtacaacgagaagttccagggcagagtgaccatcaccgccgacaagtctacctccaccgcctacatggaactgtccagcctgagatccgaggacaccgccgtgtactactgtgccagacctctgtactcctacgcctcctactattggttcgcctactggggccagggcaccctggttacagtttcttct

SEQ ID NO: 469 - skipped

SEQ ID NO: 470 - protein, 122 aa, synthetic construct
QVQLVQSGAEVKKPGSSVKVSCKISGYTFTDQTIHWVRQAPGQGLEWMGYIYPGDASAMYNEKFQGRVTITADKSTSTAYMELSSLRSEDTAVYYCARPLYSYASYYWFAYWGQGTLVTVSS

SEQ ID NO: 471 - skipped

SEQ ID NO: 472 - protein, 17 aa, synthetic construct
YIYPGDASAMYNEKFQG

SEQ ID NO: 473-477 - skipped (5 entries)

SEQ ID NO: 478 - DNA, 366 bp, synthetic construct
  source 1..366: mol_type = other DNA
caggtgcagctggttcagtctggcgccgaagtgaagaaacctggctcctccgtgaaggtgtcctgcaagatctccggctacacctttaccgaccagaccatccactgggtccgacaggctccaggacaaggcttggagtggatgggctacatctaccctggcgacgcctccgccatgtacaacgagaagtttcagggcagagtgaccatcaccgccgacaagtctacctccaccgcctacatggaactgtccagcctgagatccgaggacaccgccgtgtactactgtgccagacctctgtactcctacgcctcctactattggttcgcctactggggccagggcaccctggttacagtttcttct

SEQ ID NO: 479 - skipped

SEQ ID NO: 480 - protein, 122 aa, synthetic construct
EVQLVQSGAEVKKPGESLKISCKISGYSFTDQTIHWVHQMPGKGLEWMGYIYPGDGSAMYNEKFQGQVTISADKSISTAYLQWSSLKASDTAMYYCARPLYSYASYYWFAYWGQGTLVTVSS

SEQ ID NO: 481-487 - skipped (7 entries)

SEQ ID NO: 488 - DNA, 366 bp, synthetic construct
  source 1..366: mol_type = other DNA
gaggtgcagctggttcagtctggcgccgaagtgaagaagcctggcgagtccctgaagatctcctgcaagatcagcggctactccttcaccgaccagaccatccattgggtgcaccagatgcctggcaaaggcctggaatggatgggctacatctatcccggcgacggctccgccatgtacaacgagaagtttcagggccaagtgaccatctccgccgacaagtctatctccaccgcctacctgcagtggtcctctctgaaggcctctgacaccgctatgtactactgcgccagacctctgtactcctacgcctcctactattggttcgcctactggggccagggcaccctggttacagtttcttct

SEQ ID NO: 489 - skipped

SEQ ID NO: 490 - protein, 122 aa, synthetic construct
EVQLVQSGAEVKKPGESLKISCKGSGYTFTDQTIHWVHQMPGKGLEWMGYIYPGDGSAMYNEKFQGQVTISADKSISTAYLQWSSLKASDTAMYYCARPLYSYASYYWFAYWGQGTLVTVSS

SEQ ID NO: 491-497 - skipped (7 entries)

SEQ ID NO: 498 - DNA, 366 bp, synthetic construct
  source 1..366: mol_type = other DNA
gaggtgcagctggttcagtctggcgccgaagtgaagaagcctggcgagtccctgaagatctcctgcaaaggctccggctacaccttcaccgaccagaccattcactgggtgcaccagatgcctggcaaaggcttggagtggatgggctacatctatcccggcgacggctccgccatgtacaacgagaagtttcagggccaagtgaccatctccgccgacaagtctatctccaccgcctacctgcagtggtcctctctgaaggcctctgacaccgctatgtactactgcgccagacctctgtactcctacgcctcctactattggttcgcctactggggccagggcaccctggttacagtttcttct

SEQ ID NO: 499 - skipped

SEQ ID NO: 500 - protein, 122 aa, synthetic construct
EVQLVQSGAEVKKPGESLKISCKGSGYSFTDQTIHWVHQMPGKGLEWMGYIYPGDGSAMYNEKFQGQVTISADKSISTAYLQWSSLKASDTAMYYCARPLYSYASYYWFAYWGQGTLVTVSS

SEQ ID NO: 501-507 - skipped (7 entries)

SEQ ID NO: 508 - DNA, 366 bp, synthetic construct
  source 1..366: mol_type = other DNA
gaggtgcagctggttcagtctggcgccgaagtgaagaagcctggcgagtccctgaagatctcctgcaaaggctccggctactccttcaccgaccagaccattcactgggtgcaccagatgcctggcaaaggcttggagtggatgggctacatctatcccggcgacggctccgccatgtacaacgagaagtttcagggccaagtgaccatctccgccgacaagtctatctccaccgcctacctgcagtggtcctctctgaaggcctctgacaccgctatgtactactgcgccagacctctgtactcctacgcctcctactattggttcgcctactggggccagggcaccctggttacagtttcttct

SEQ ID NO: 509 - skipped

SEQ ID NO: 510 - protein, 122 aa, synthetic construct
EVQLVQSGAEVKKPGESLKISCKISGYTFTDQTIHWVHQMPGKGLEWMGYIYPGDSSAMYNEKFQGQVTISADKSISTAYLQWSSLKASDTAMYYCARPLYSYASYYWFAYWGQGTLVTVSS

SEQ ID NO: 511-517 - skipped (7 entries)

SEQ ID NO: 518 - DNA, 366 bp, synthetic construct
  source 1..366: mol_type = other DNA
gaggtgcagctggttcagtctggcgccgaagtgaagaagcctggcgagtccctgaagatctcctgcaagatcagcggctacaccttcaccgaccagaccatccattgggtgcaccagatgcctggcaaaggcctggaatggatgggctacatctaccccggcgactcctccgccatgtacaacgagaagttccagggccaagtgaccatctccgccgacaagtctatctccaccgcctacctgcagtggtcctctctgaaggcctctgacaccgctatgtactactgcgccagacctctgtactcctacgcctcctactattggttcgcctactggggccagggcaccctggttacagtttcttct

SEQ ID NO: 519 - skipped

SEQ ID NO: 520 - protein, 122 aa, synthetic construct
EVQLVQSGAEVKKPGESLKISCKISGYSFTDQTIHWVHQMPGKGLEWMGYIYPGDSSAMYNEKFQGQVTISADKSISTAYLQWSSLKASDTAMYYCARPLYSYASYYWFAYWGQGTLVTVSS

SEQ ID NO: 521-527 - skipped (7 entries)

SEQ ID NO: 528 - DNA, 366 bp, synthetic construct
  source 1..366: mol_type = other DNA
gaggtgcagctggttcagtctggcgccgaagtgaagaagcctggcgagtccctgaagatctcctgcaagatcagcggctactccttcaccgaccagaccatccattgggtgcaccagatgcctggcaaaggcctggaatggatgggctacatctaccccggcgactcctccgccatgtacaacgagaagttccagggccaagtgaccatctccgccgacaagtctatctccaccgcctacctgcagtggtcctctctgaaggcctctgacaccgctatgtactactgcgccagacctctgtactcctacgcctcctactattggttcgcctactggggccagggcaccctggttacagtttcttct

SEQ ID NO: 529 - skipped

SEQ ID NO: 530 - protein, 122 aa, synthetic construct
QVQLVQSGAEVKKPGSSVKVSCKASGYTFTDQTIHWVRQAPGQGLEWMGYIYPGDSSAMYNEKFQGRVTITADKSTSTAYMELSSLRSEDTAVYYCARPLYSYASYYWFAYWGQGTLVTVSS

SEQ ID NO: 531-537 - skipped (7 entries)

SEQ ID NO: 538 - DNA, 366 bp, synthetic construct
  source 1..366: mol_type = other DNA
caggtgcagctggttcagtctggcgccgaagtgaagaaacctggctcctccgtgaaggtgtcctgcaaggcttctggctacacctttaccgaccagaccatccactgggtccgacaggctccaggacaaggcttggagtggatgggctacatctatcctggcgacagctccgccatgtacaacgagaagtttcagggcagagtgaccatcaccgccgacaagtctacctccaccgcctacatggaactgtccagcctgagatctgaggacaccgccgtgtactactgcgccagacctctgtactcctacgcctcctactattggttcgcctactggggccagggcaccctggttacagtttcttct

SEQ ID NO: 539 - skipped

SEQ ID NO: 540 - protein, 122 aa, synthetic construct
QVQLVQSGAEVKKPGSSVKVSCKASGYTFTDQTIHWVRQAPGQGLEWMGYIYPGDASAMYNEKFQGRVTITADKSTSTAYMELSSLRSEDTAVYYCARPLYSYASYYWFAYWGQGTLVTVSS

SEQ ID NO: 541-547 - skipped (7 entries)

SEQ ID NO: 548 - DNA, 366 bp, synthetic construct
  source 1..366: mol_type = other DNA
caggtgcagctggttcagtctggcgccgaagtgaagaaacctggctcctccgtgaaggtgtcctgcaaggcttctggctacacctttaccgaccagaccatccactgggtccgacaggctccaggacaaggcttggagtggatgggctacatctatcctggcgacgcctccgccatgtacaacgagaagtttcagggcagagtgaccatcaccgccgacaagtctacctccaccgcctacatggaactgtccagcctgagatctgaggacaccgccgtgtactactgcgccagacctctgtactcctacgcctcctactattggttcgcctactggggccagggcaccctggttacagtttcttct

SEQ ID NO: 549-557 - skipped (9 entries)

SEQ ID NO: 558 - DNA, 366 bp, synthetic construct
  source 1..366: mol_type = other DNA
caggtccagctgcagcagtctgatgccgagcttgtgaagcctggcgcctccgtgaagatctcctgcaagatctccggctacaccttcaccgaccagaccatccactggatgaaccagaggcctgagcagggccttgagtggatcggctacatctatcctggcgacggctccgccatgtacaacgagaagttcagaggcaaggctaccctgaccgccgacaagtcctcttccaccgcttacatgcagctgaactccctgacctccgaggactccgccgtgtacttttgtgcccggcctctgtactcctacgcctcctactattggttcgcctactggggccagggcaccctggttacagtttctgct

SEQ ID NO: 559 - DNA, 321 bp, synthetic construct
  source 1..321: mol_type = other DNA
gacatccagatgaaccagtctccatccagcctgtctgccagcctgggcgacaccatcaccatcacatgtcacgccagccagaacatcaacgtgtggctgtcctggtatcagcggaagcccggcaacatccccaagctgctgatctacaagtcctccaacctgcacaccggcgtgccctctagattttccggctctggctctggcaccggctttaccctgacaatcagctccctgcagcctgaggatatcgccacctactactgtcagcagggccagagctaccctctgacatttggcggcggaacaaagctggaaatcaag